(19) United States
(12) Patent Application Publication
(10) Pub. No.: US 2024/0208971 A1
(43) Pub. Date: Jun. 27, 2024

(54) INHIBITORS OF KINASE NETWORKS AND USES THEREOF

(71) Applicant: Purdue Research Foundation, West Lafayette, IN (US)

(72) Inventors: Herman O. Sintim, West Lafayette, IN (US); Neetu Dayal, West Lafayette, IN (US); Clemen Opoku-Temeng, West Lafayette, IN (US)

(21) Appl. No.: 18/392,204

(22) Filed: Dec. 21, 2023

Related U.S. Application Data
(62) Division of application No. 17/243,629, filed on Apr. 29, 2021, now Pat. No. 11,891,391.
Application No. 17/243,629 is a division of application No. 16/499,133, filed on Sep. 27, 2019, now Pat. No. 11,040,973.
(60) Provisional application No. 62/478,069, filed on Mar. 29, 2017.
(60) Provisional application No. 62/616,643, filed on Jan. 12, 2018.

(51) Int. Cl. C07D 471/14; C07D 209/08; C07D 231/56; C07D 401/14; C07D 413/14; C07D 417/12; C07D 471/04; C07D 491/147; C07D 495/14; C07D 498/04; C07D 513/04; C07D 519/00

(52) U.S. Cl. CPC C07D 471/14; C07D 209/08; C07D 231/56; C07D 401/14; C07D 413/14; C07D 417/12; C07D 471/04; C07D 491/147; C07D 495/14; C07D 498/04; C07D 513/04; C07D 519/00; C07B 2200/05

(57) ABSTRACT

The present invention generally relates to compounds as a dual kinase-demethylase inhibitor useful for the treatment of diseases mediated by a kinase and/or a histone demethylase, such as inflammation, cancer, viral and bacterial infections, neurological and immunological disorders. Pharmaceutical compositions and methods for treating those diseases are within the scope of this invention.

### CROSS REFERENCE TO RELATED APPLICATIONS

This present U.S. patent application relates to and claims the priority benefit of U.S. Provisional Application Ser. No. 62/478,069, filed Mar. 29, 2017, and U.S. Provisional Application Ser. No. 62/616,643, filed Jan. 12, 2018, the contents of which are hereby incorporated herein by reference in their entirety.

### TECHNICAL FIELD

The present invention generally relates to compounds as a kinase inhibitor and methods for the treatment of diseases mediated by a kinase, such as inflammation, cancer, viral and bacterial infections, neurological and immunological disorders.

### BACKGROUND

This section introduces aspects that may help facilitate a better understanding of the disclosure. Accordingly, these statements are to be read in this light and are not to be understood as admissions about what is or is not prior art.

The cell contains over 500 kinases, which regulate diverse processes such as cell cycle, growth, migration, immune response.¹ Several deregulated kinases, i.e. kinases that have attained a gain-of-function mutation or are over-expressed, drive cancer proliferation.¹ Small molecule inhibitors of cancer-driver kinases (for example BCR-ABL1 fusion protein, FLT3-ITD, mutated or over-expressed ALK. EGFR, PDGFR. Kit. VEGFR. B-Raf, BTK, PI3Kδ, ErbB2) have seen clinical successes.² Recently efforts have been made to target other kinases, such as cell cycle kinases (CDKs), or kinases that target histones, cytoskeleton or other processes that are important for the cell, to arrest cancer growth. Most of the kinase inhibitors that proceed to the clinic work initially but over time resistant clones emerge that render the drugs ineffective.³ Various mechanisms account for cancer cell resistance to kinase inhibitors. For example copy number multiplication, additional kinase mutations (such as secondary mutations that arise in in the tyrosine kinase domain of FLT3-ITD kinase) or the activation of alternative kinase pathways and/or downstream targets can bypass the inhibition of a particular kinase target.⁴ Kinase inhibitors that inhibit a cancer-driver kinase and also downstream targets (both kinase and non-kinase targets, such as histone demethylase) and/or kinases that collaborate with the driver kinase could have enhanced potency and reduced probability of resistance being generated against that kinase inhibitor.⁵ A challenge however with such a polypharmacophore is to avoid promiscuous binding, which can lead to toxicity.

Kinase inhibitors have also been shown to be effective for the treatment of immune disorders (such as JAK kinases⁶), hypertension and erectile dysfunction (ROCK1/2 kinases⁷) and glaucoma (ROCK and LIMK kinases⁸). Other kinase targets, such as LRRK2, have also been shown to be important for CNS-related diseases, such as Alzheimer's or Parkinsons.⁹ A privileged chemical scaffold, which can be tuned to selectively inhibit a disease-related kinase or inhibit a group of kinases that lie in a particular pathway or network could facilitate the treatment of diverse disease states.

### SUMMARY OF THE INVENTION

The present invention generally relates to compounds that inhibit kinase and/or histone demethylase networks as useful compounds for the treatment of diseases mediated by a kinase, such as inflammation, cancer, viral and bacterial infections, neurological and immunological disorders. Pharmaceutical compositions and methods for treating those diseases are within the scope of this invention.

In some illustrative embodiments, the present invention relates to a compound having a formula

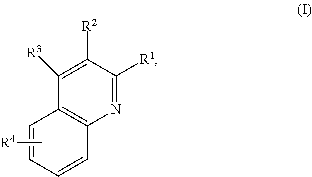

or a pharmaceutically acceptable salt thereof, wherein
R¹ is amino, hydroxyl, and derivatives thereof, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted;
R² and R³ are each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted;
or R² and R³ are taken together with the attached carbons to form an optionally substituted cyclic or heterocyclic moiety; and
R⁴ represents four substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; or any two adjacent substituents of the four substituents are taken together with the attached carbons form an optionally substituted cyclic or heterocyclic moiety.

In some illustrative embodiments, the present invention relates to a compound having a formula:

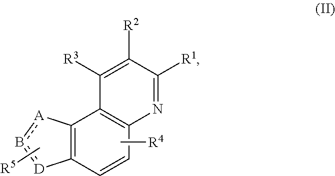

or a pharmaceutically acceptable salt thereof, wherein n=1˜5; the bonding between A and B, between B and D may be a double bond or a single bond, but cannot be double bond at the same time; A, B, and D represents, independently, C, O, N, and S wherein at least one of A, B, and D is a heteroatom;
R¹ is amino, hydroxyl, and derivatives thereof, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted;
R² and R³ are each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; or R² and R³ are taken together with the attached carbons to form an optionally substituted cyclic or heterocyclic moiety;
R⁴ represents two substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; or the two substituents are taken together with the attached carbons form an optionally substituted cyclic or heterocyclic moiety; and
depending on the element of A, B and D, R⁵ represents two or three substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted.

In some illustrative embodiments, the present invention relates to a compound having a formula (II), wherein
R¹ is:

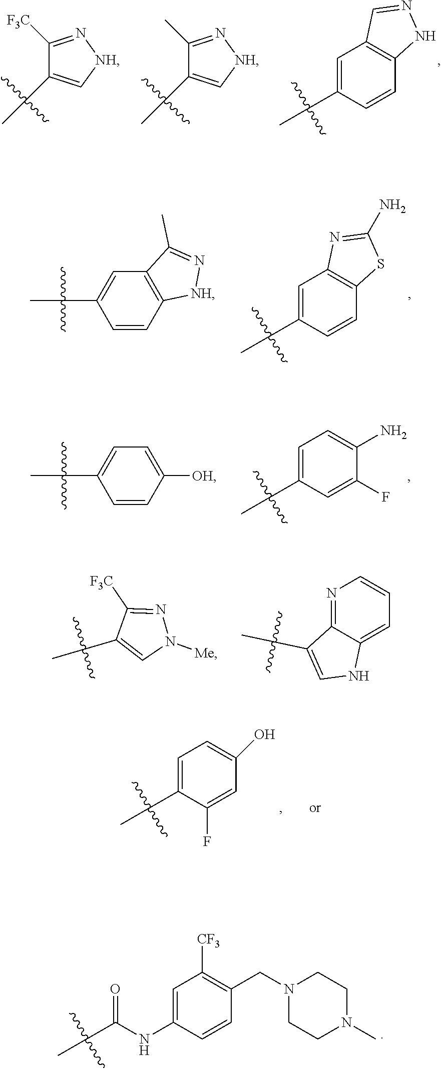

In some illustrative embodiments, the present invention relates to a compound having a formula (III).

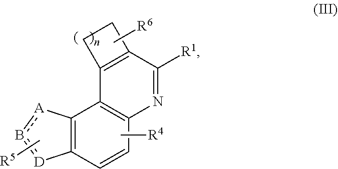

or a pharmaceutically acceptable salt thereof, wherein n=1˜5: the bonding between A and B, between B and D may be a double bond or a single bond, but cannot be double bond at the same time; A, B, and D represents, independently, C, O, N, and S wherein at least one of A, B, and D is a heteroatom;
R¹ is amino, hydroxyl, and derivatives thereof, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted;
R⁴ represents two substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; or the two substituents are taken together with the attached carbons form an optionally substituted cyclic or heterocyclic moiety.
depending on the element of A, B and D, R⁵ represents two or three substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; and
depending on the value of n, R⁶ represents two to six substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted.

In some illustrative embodiments, the present invention relates to a compound having a formula (III), wherein R¹ is

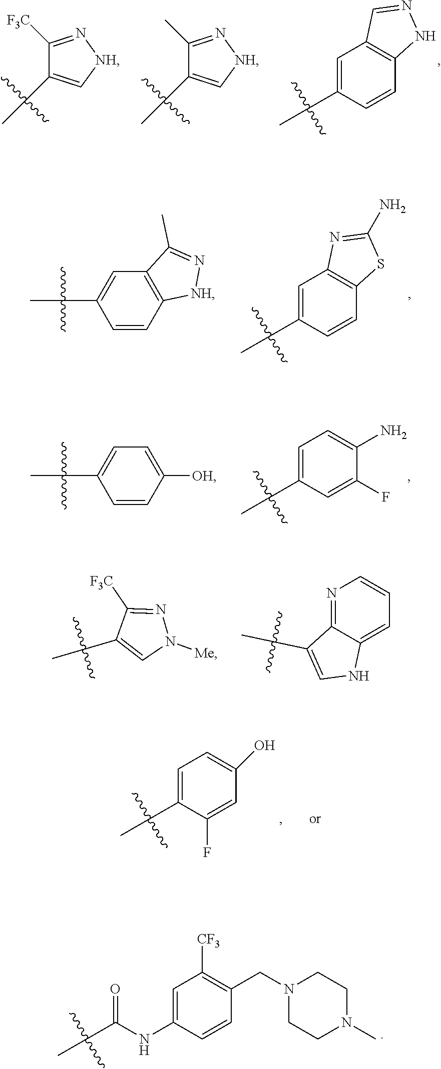

In some illustrative embodiments, the present invention relates to a compound having a formula (IV),

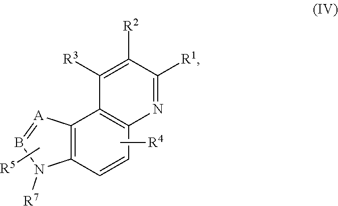

or a pharmaceutically acceptable salt thereof, wherein n=1˜5;
R¹ is amino, hydroxyl, and derivatives thereof, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted;
R² and R³ are each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; or R² and R³ are taken together with the attached carbons to form an optionally substituted cyclic or heterocyclic moiety;
R⁴ represents two substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; or the two substituents are taken together with the attached carbons form an optionally substituted cyclic or heterocyclic moiety;
depending on the element of A and B specified below. R⁵ represents one or two substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted;
A and B represents, independently, CR⁸, N, or NR⁹, wherein R⁸ and R⁹ represent independently hydrogen, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted;
depending on the value of n, R⁶ represents two to six substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; and
R⁷ is an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a compound having a formula (IV), wherein R¹ is

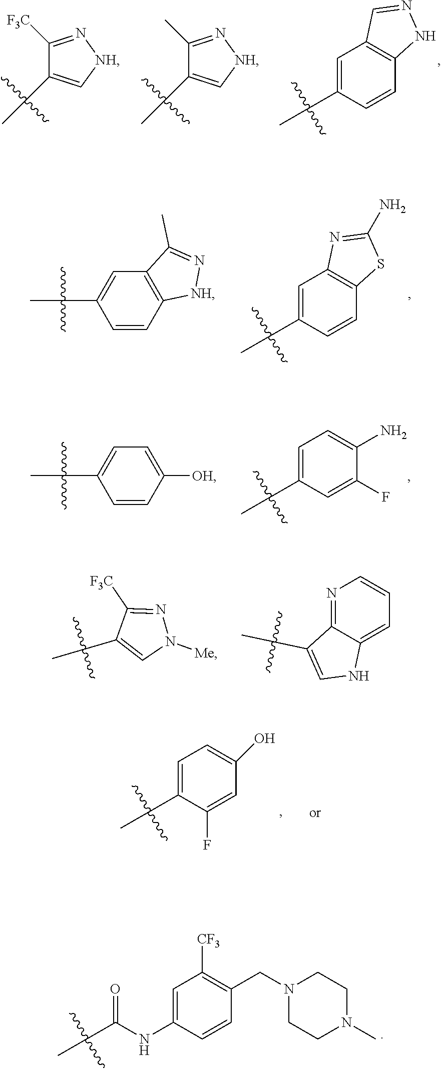

In some illustrative embodiments, the present invention relates to a compound having a formula (IV), wherein R¹ is

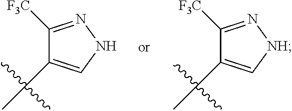 [SMILES: *c1c[nH]nc1C(F)(F)F ; *c1c[nH]nc1C(F)(F)F]

A is carbon (C); B is nitrogen (N); R⁵, R⁶, and R⁷ all represent hydrogen, and R⁴ represents two substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted;
or the two substituents are taken together with the attached carbons form an optionally substituted cyclic or heterocyclic moiety.

In some illustrative embodiments, the present invention relates to a compound having a formula (IV), wherein R³ is

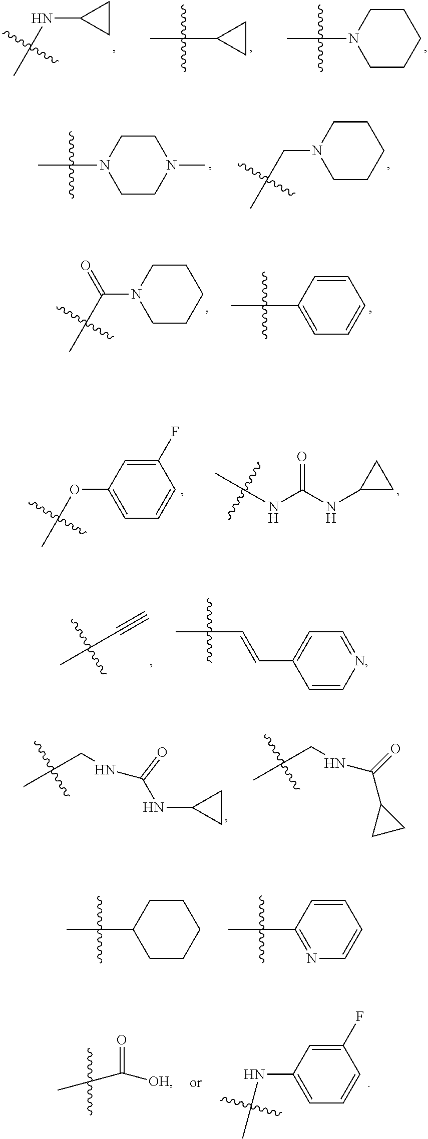

In some illustrative embodiments, the present invention relates to a compound having a formula (IV), wherein R¹ is

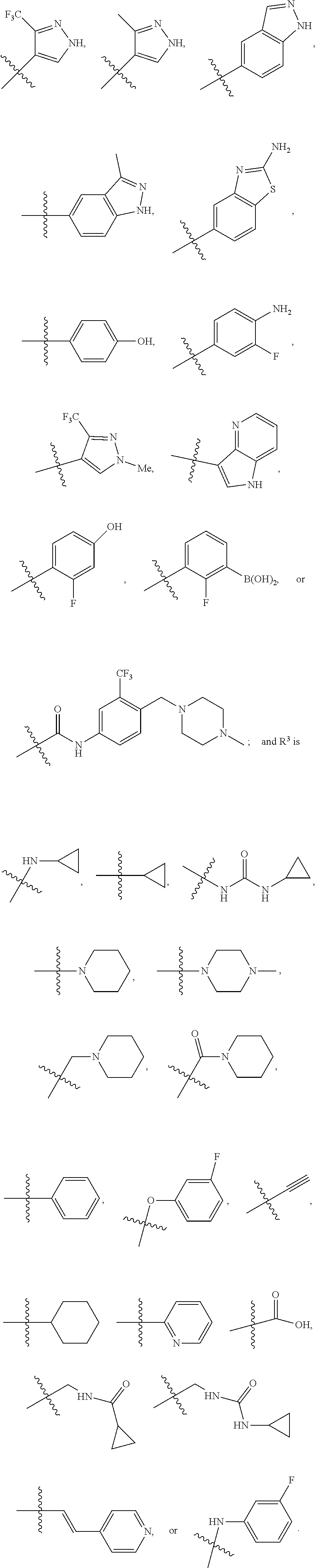

In some illustrative embodiments, the present invention relates to a compound having a formula (I), wherein the compound is

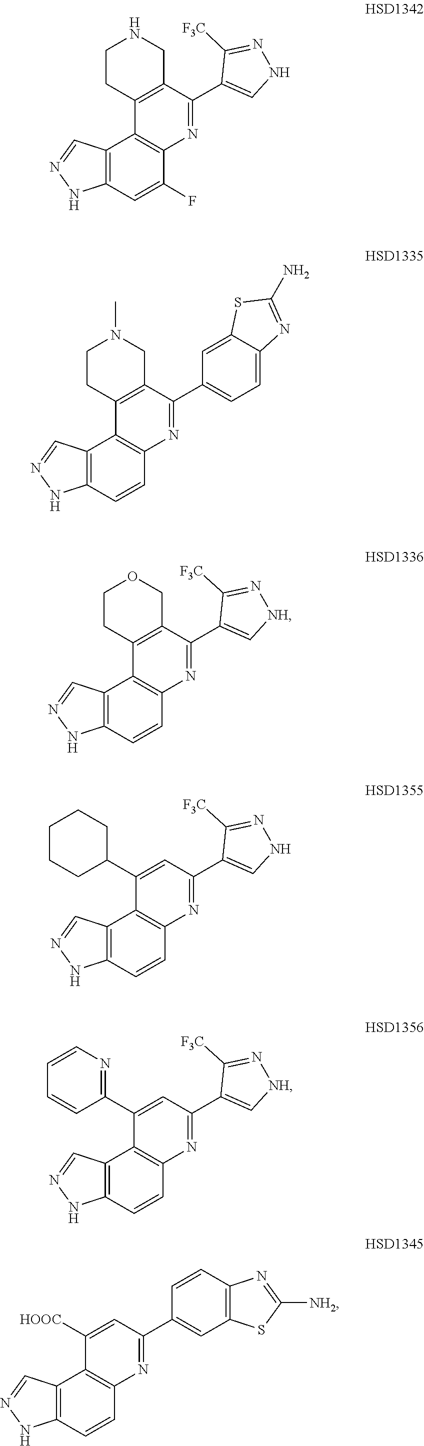

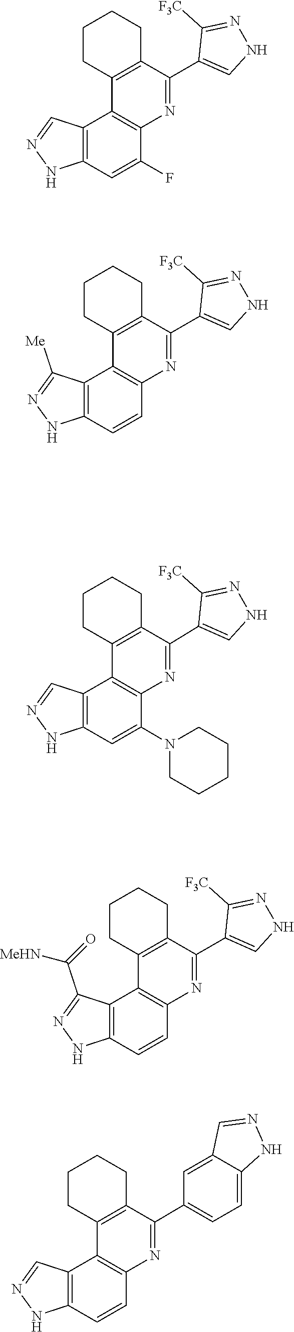 [SMILES: Fc1cc2[nH]ncc2c2c3c(c(-c4c[nH]nc4C(F)(F)F)nc12)CCCC3 ; Cc1n[nH]c2ccc3nc(-c4c[nH]nc4C(F)(F)F)c4c(c3c12)CCCC4 ; FC(F)(F)c1n[nH]cc1-c1nc2c(N3CCCCC3)cc3[nH]ncc3c2c2c1CCCC2 ; CNC(=O)c1n[nH]c2ccc3nc(-c4c[nH]nc4C(F)(F)F)c4c(c3c12)CCCC4 ; c1cc2[nH]ncc2cc1-c1nc2ccc3[nH]ncc3c2c2c1CCCC2]

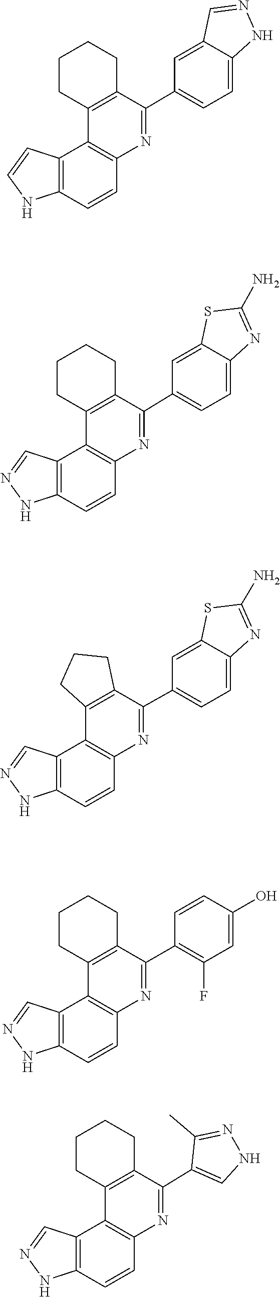 [SMILES: c1cc2c(ccc3nc(-c4ccc5[nH]ncc5c4)c4c(c32)CCCC4)[nH]1 ; Nc1nc2ccc(-c3nc4ccc5[nH]ncc5c4c4c3CCCC4)cc2s1 ; Nc1nc2ccc(-c3nc4ccc5[nH]ncc5c4c4c3CCC4)cc2s1 ; Oc1ccc(-c2nc3ccc4[nH]ncc4c3c3c2CCCC3)c(F)c1 ; Cc1n[nH]cc1-c1nc2ccc3[nH]ncc3c2c2c1CCCC2]

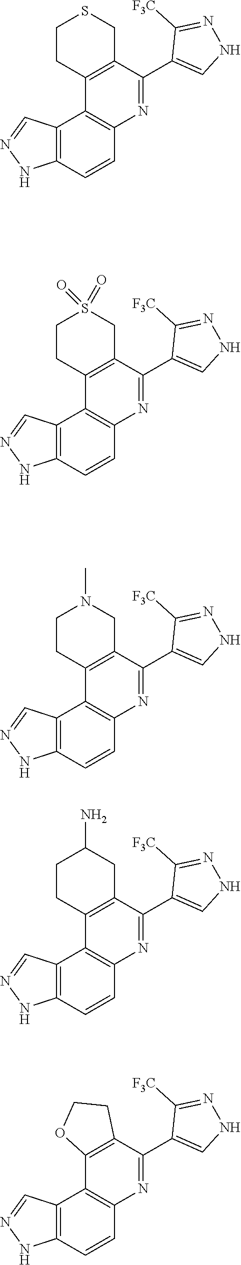

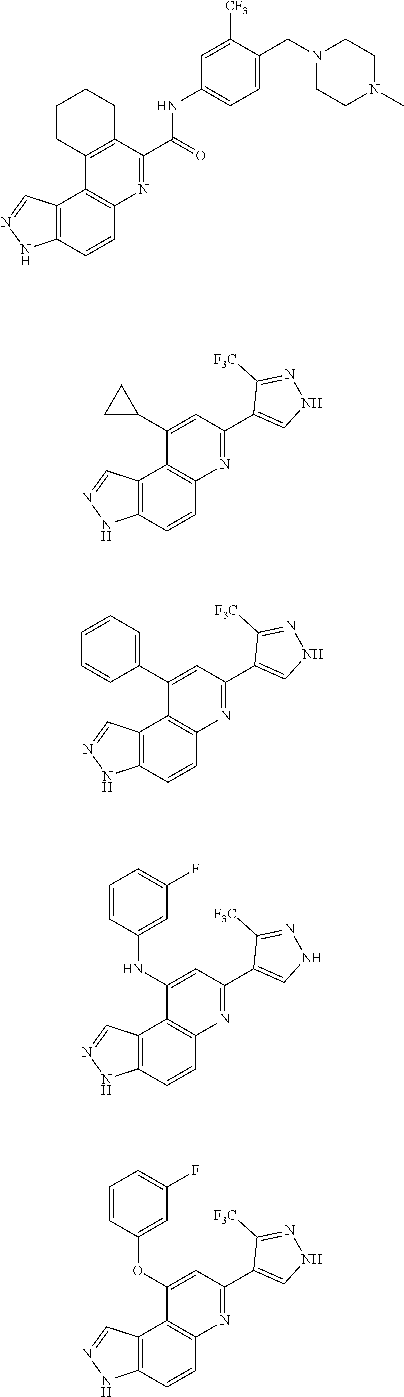 [SMILES: CN1CCN(Cc2ccc(NC(=O)c3nc4ccc5[nH]ncc5c4c4c3CCCC4)cc2C(F)(F)F)CC1 ; FC(F)(F)c1n[nH]cc1-c1cc(C2CC2)c2c(ccc3[nH]ncc32)n1 ; FC(F)(F)c1n[nH]cc1-c1cc(-c2ccccc2)c2c(ccc3[nH]ncc32)n1 ; Fc1cccc(Nc2cc(-c3c[nH]nc3C(F)(F)F)nc3ccc4[nH]ncc4c23)c1 ; Fc1cccc(Oc2cc(-c3c[nH]nc3C(F)(F)F)nc3ccc4[nH]ncc4c23)c1]

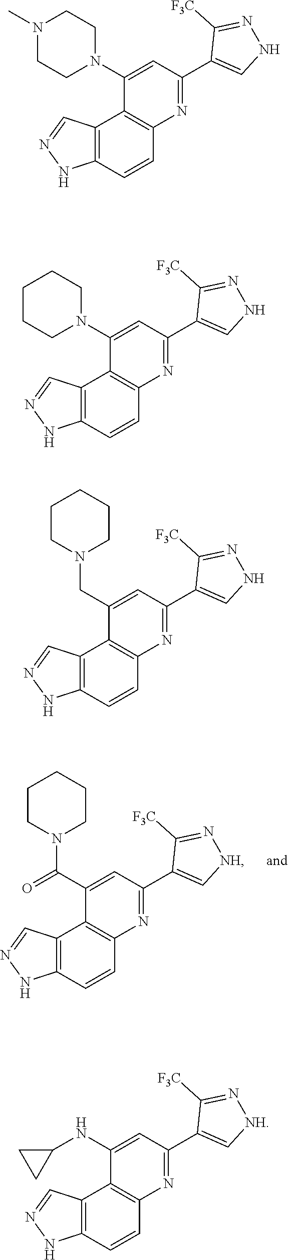 [SMILES: CN1CCN(c2cc(-c3c[nH]nc3C(F)(F)F)nc3ccc4[nH]ncc4c23)CC1 ; FC(F)(F)c1n[nH]cc1-c1cc(N2CCCCC2)c2c(ccc3[nH]ncc32)n1 ; FC(F)(F)c1n[nH]cc1-c1cc(CN2CCCCC2)c2c(ccc3[nH]ncc32)n1 ; O=C(c1cc(-c2c[nH]nc2C(F)(F)F)nc2ccc3[nH]ncc3c12)N1CCCCC1 ; FC(F)(F)c1n[nH]cc1-c1cc(NC2CC2)c2c(ccc3[nH]ncc32)n1]

In some illustrative embodiments, the present invention relates to a pharmaceutical composition comprising one or more compounds disclosed herein, or a pharmaceutically acceptable salt thereof, together with one or more diluents, excipients or carriers.

In some illustrative embodiments, the present invention relates to a kinase inhibitor, wherein the kinase is selected from the group consisting of FLT3, MNK1/2, JAK1/2/3, Limk1/2, various CDKs, Haspin, ROCK1/2, TOPK, LRRK2. GSK3a/3b, RSK1-4, ERK, P7056K, AKT, PI3K, p38, PKC, PKA, FGFR1-4. VEGFR1-3, ALK, AXL, LIMK1/2, Aurora A/B, ABL1. AKT, CSF1R, CSNK1D, DCAMKL1, CSNKIG2. EPHA2. ERBB2, IKK-alpha, IKK-beta, JNK1/2/3, MARK3, MEK1/2, MET. MLK1. PAK1/2/4, PDGFRa/b, PIM1/2/3, PLK1/2/3/4. PRKCE, PRKX, RET, TAOK2, TRKA/B/C, ULK2, and receptor-interacting protein kinase 4 (RIPK4).

In some illustrative embodiments, the present invention relates to a method for treating diseases mediated by a kinase and/or histone demethylases, including inflammation, cancer, viral and bacterial infections, gastrointestinal disorders, eye diseases, neurological, cardiovascular and immunological disorders, comprising the step of administering a therapeutically effective amount of one or more compounds disclosed herein, and one or more carriers, diluents, or excipients, to a patient in need of relief from said cancer.

In some illustrative embodiments, the present invention relates to a method for treating diseases mediated by a kinase and histone demethylases, including inflammation, cancer, viral and bacterial infections, gastrointestinal disorders, eye diseases, neurological, cardiovascular and immunological disorders, comprising the step of administering a therapeutically effective amount of a compound disclosed herein in combination with one or more other compounds of the same or different mode of action, and one or more carriers, diluents, or excipients, to a patient in need of relief from said cancer.

These and other features, aspects and advantages of the present invention will become better understood with reference to the following detailed description and claims.

### DETAILED DESCRIPTION

While the concepts of the present disclosure are illustrated and described in detail in the description herein, results in the their description are to be considered as exemplary and not restrictive in character; it being understood that only the illustrative embodiments are shown and described and that all changes and modifications that come within the spirit of the disclosure are desired to be protected.

The present invention generally relates to compounds as kinase inhibitor useful for the treatment of diseases mediated by a kinase, such as inflammation, cancer, viral and bacterial infections, neurological and immunological disorders. Pharmaceutical compositions and methods for treating those diseases are within the scope of this invention.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “substituted” as used herein refers to a functional group in which one or more hydrogen atoms contained therein are replaced by one or more non-hydrogen atoms. The term “functional group” or “substituent” as used herein refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

The term “alkyl” as used herein refers to substituted or unsubstituted straight chain and branched alkyl groups and cycloalkyl groups having from 1 to about 20 carbon atoms (C₁-C₂₀₎, 1 to 12 carbons (C₁-C₁₂), 1 to 8 carbon atoms (C₁-C₈), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to substituted or unsubstituted straight chain and branched divalent alkenyl and cycloalkenyl groups having from 2 to 20 carbon atoms (C₂-C₂₀), 2 to 12 carbons (C₂-C₁₂), 2 to 8 carbon atoms (C₂-C₈) or, in some embodiments, from 2 to 4 carbon atoms (C₂-C₄) and at least one carbon-carbon double bond. Examples of straight chain alkenyl groups include those with from 2 to 8 carbon atoms such as —CH═CH—, —CH═CHCH₂—, and the like. Examples of branched alkenyl groups include, but are not limited to, —CH═C(CH₃)— and the like.

An alkynyl group is the fragment, containing an open point of attachment on a carbon atom that would form if a hydrogen atom bonded to a triply bonded carbon is removed from the molecule of an alkyne. The term “hydroxyalkyl” as used herein refers to alkyl groups as defined herein substituted with at least one hydroxyl (—OH) group.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aryl” as used herein refers to substituted or unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to substituted or unsubstituted aromatic and non-aromatic ring compounds containing 3 or more ring members, of which, one or more is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈).

A heteroaryl ring is an embodiment of a heterocyclyl group. The phrase “heterocyclyl group” includes fused ring species including those that include fused aromatic and non-aromatic groups. Representative heterocyclyl groups include, but are not limited to pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having. e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R³N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

The term “optionally substituted,” or “optional substituents,” as used herein, means that the groups in question are either unsubstituted or substituted with one or more of the substituents specified. When the groups in question are substituted with more than one substituent, the substituents may be the same or different. When using the terms “independently,” “independently are,” and “independently selected from” mean that the groups in question may be the same or different. Certain of the herein defined terms may occur more than once in the structure, and upon such occurrence each term shall be defined independently of the other.

The compounds described herein may contain one or more chiral centers, or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar, (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the phannacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient; the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

In some illustrative embodiments, the present invention relates to a compound having a

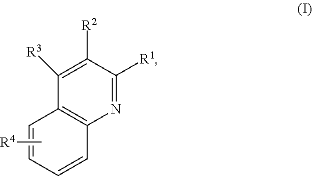

or a pharmaceutically acceptable salt thereof, wherein
R¹ is amino, hydroxyl, and derivatives therof, an alkyl, alkenyl, alkynyl, heteroalkyl, heteroalkenyl, heteroalkynyl, heterocyclyl, cycloalkyl, cycloalkenyl, cyclohetermalkyl, cycloheteroalkenyl, acyl, aryl, heteroaryl, arylalkyl, arylalkenyl, or arylalkynyl, each of which is optionally substituted;
R² and R³ are each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted;
or R² and R³ are taken together with the attached carbons to form an optionally substituted cyclic or heterocyclic moiety; and
R⁴ represents four substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; or any two adjacent substituents of the four substituents are taken together with the attached carbons form an optionally substituted cyclic or heterocyclic moiety.

In some illustrative embodiments, the present invention relates to a compound having a formula:

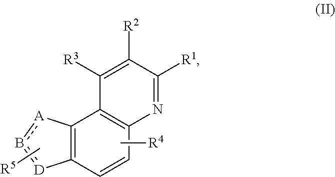

or a pharmaceutically acceptable salt thereof, wherein n=1˜5; the bonding between A and B, between B and D may be a double bond or a single bond, but cannot be double bond at the same time; A, B, and D represents, independently, C, O, N, and S wherein at least one of A, B, and D is a heteroatom;
R¹ is amino, hydroxyl, and derivatives thereof, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted;
R² and R³ are each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; or R² and R³ are taken together with the attached carbons to form an optionally substituted cyclic or heterocyclic moiety;
R⁴ represents two substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; or the two substituents are taken together with the attached carbons form an optionally substituted cyclic or heterocyclic moiety; and
depending on the element of A, B and D, R⁵ represents two or three substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted.

In some illustrative embodiments, the present invention relates to a compound having a formula (II), wherein
R¹ is:

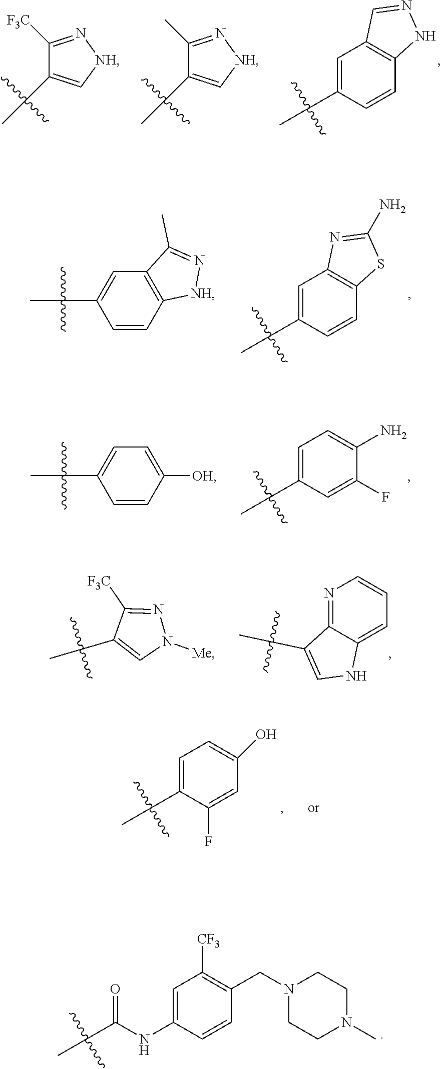

In some illustrative embodiments, the present invention relates to a compound having a formula (III),

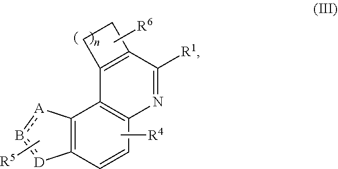

or a pharmaceutically acceptable salt thereof, wherein n=1˜5; the bonding between A and B, between B and D may be a double bond or a single bond, but cannot be double bond at the same time; A, B, and D represents, independently. C, O, N, and S wherein at least one of A, B, and D is a heteroatom;
R¹ is amino, hydroxyl, and derivatives thereof, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted;
R⁴ represents two substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; or the two substituents are taken together with the attached carbons form an optionally substituted cyclic or heterocyclic moiety.
depending on the element of A, B and D, R⁵ represents two or three substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; and
depending on the value of n, R⁶ represents two to six substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted.

In some illustrative embodiments, the present invention relates to a compound having a formula (III), wherein R¹ is

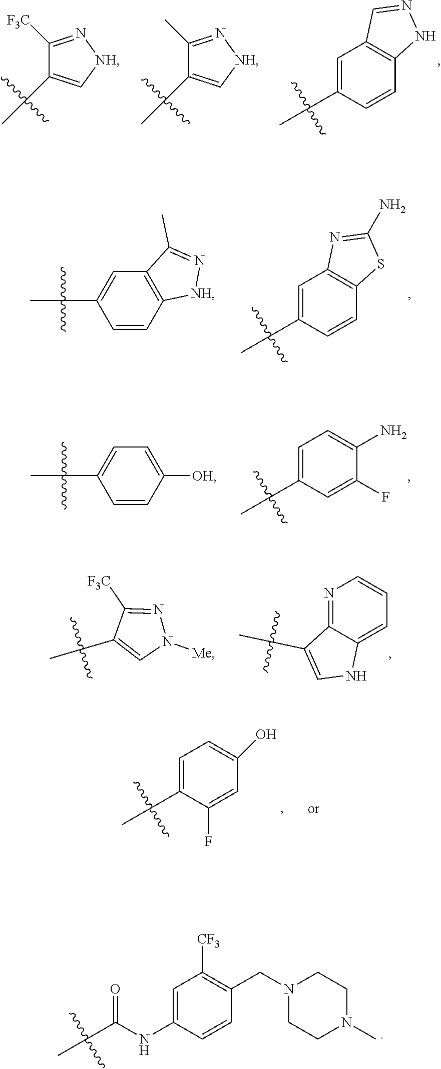

In some illustrative embodiments, the present invention relates to a compound having a formula (IV).

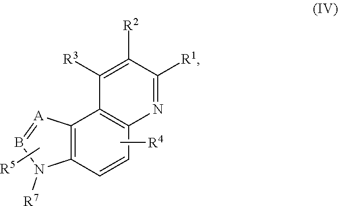

or a pharmaceutically acceptable salt thereof, wherein n=1˜5;
R¹ is amino, hydroxyl, and derivatives thereof, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted;
R² and R³ are each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; or R² and R³ are taken together with the attached carbons to form an optionally substituted cyclic or heterocyclic moiety;
R⁴ represents two substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; or the two substituents are taken together with the attached carbons form an optionally substituted cyclic or heterocyclic moiety:
depending on the element of A and B specified below, R⁵ represents one or two substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted;
A and B represents, independently. CR⁸, N, or NR⁹, wherein R⁸ and R⁹ represent independently hydrogen, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted;
depending on the value of n, R⁶ represents two to six substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; and
R⁷ is an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

In some illustrative embodiments, the present invention relates to a compound having a formula (IV), wherein R¹ is

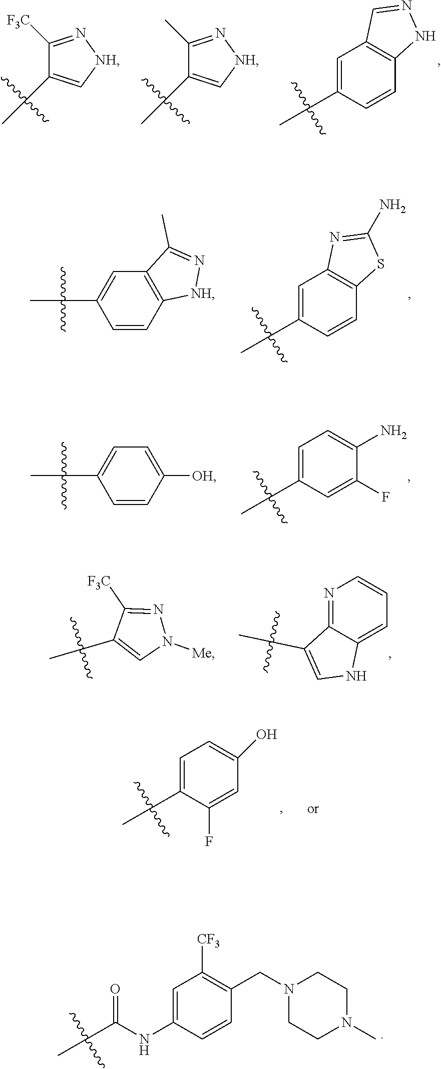

In some illustrative embodiments, the present invention relates to a compound having a formula (IV), wherein R¹ is

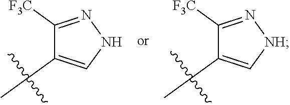 [SMILES: *c1c[nH]nc1C(F)(F)F ; "or" ; *c1c[nH]nc1C(F)(F)F]

A is carbon (C); B is nitrogen (N); R⁵, R⁶, and R⁷ all represent hydrogen, and R⁴ represents two substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; or the two substituents are taken together with the attached carbons form an optionally substituted cyclic or heterocyclic moiety.

In some illustrative embodiments, the present invention relates to a compound having a formula (IV), wherein R³,

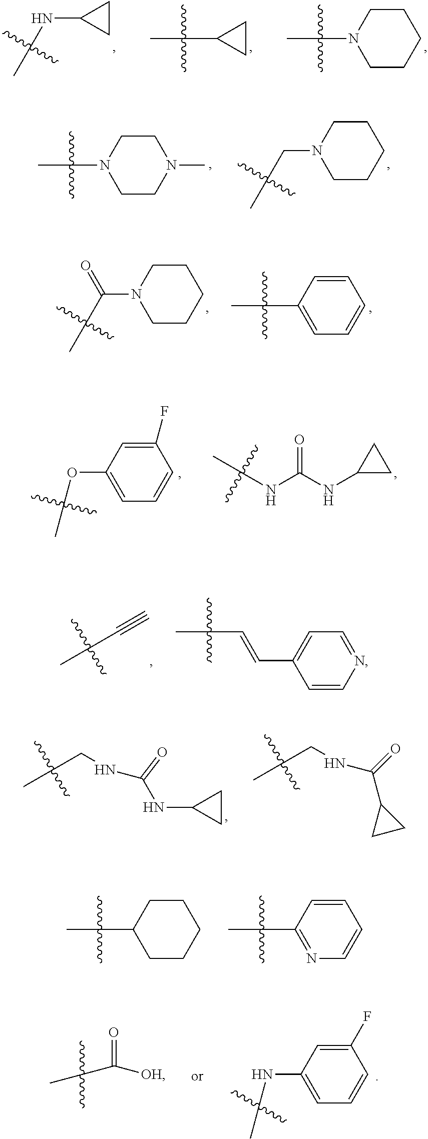

In some illustrative embodiments, the present invention relates to a compound having a formula (IV), wherein R¹ is

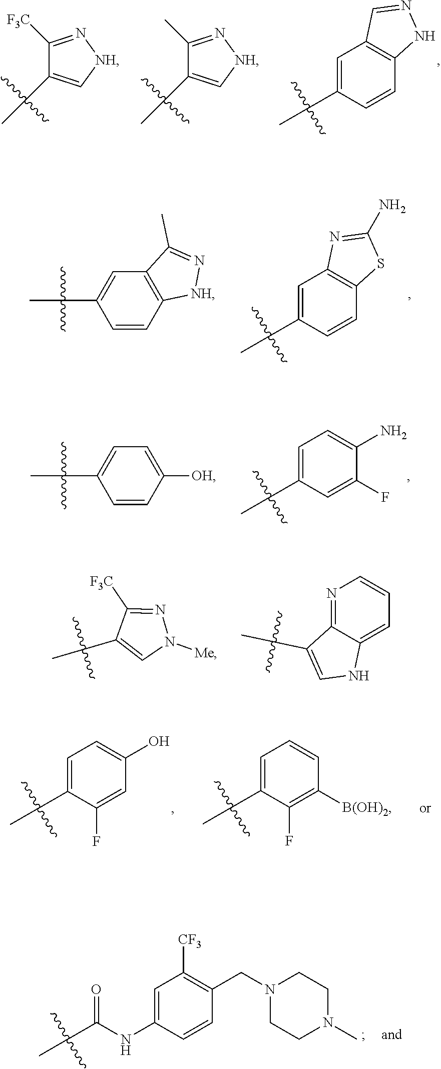

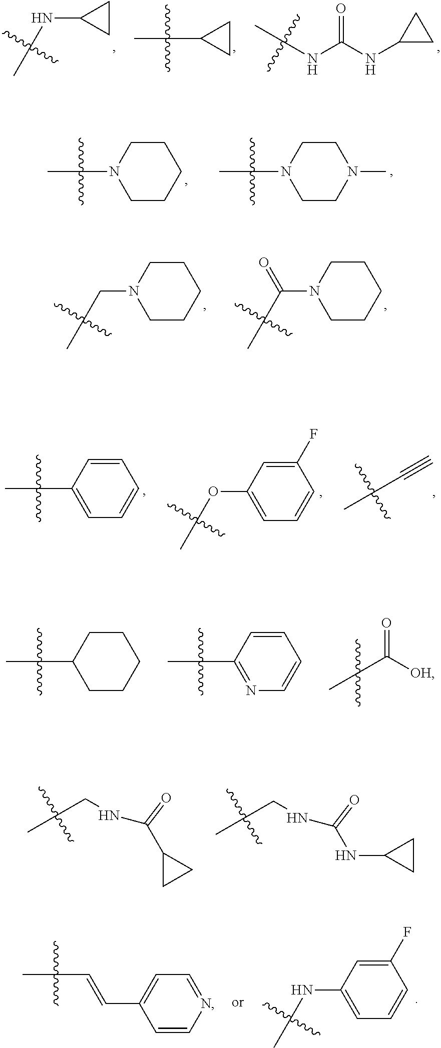

In some illustrative embodiments, the present invention relates to a compound having a formula (I), wherein the compound is

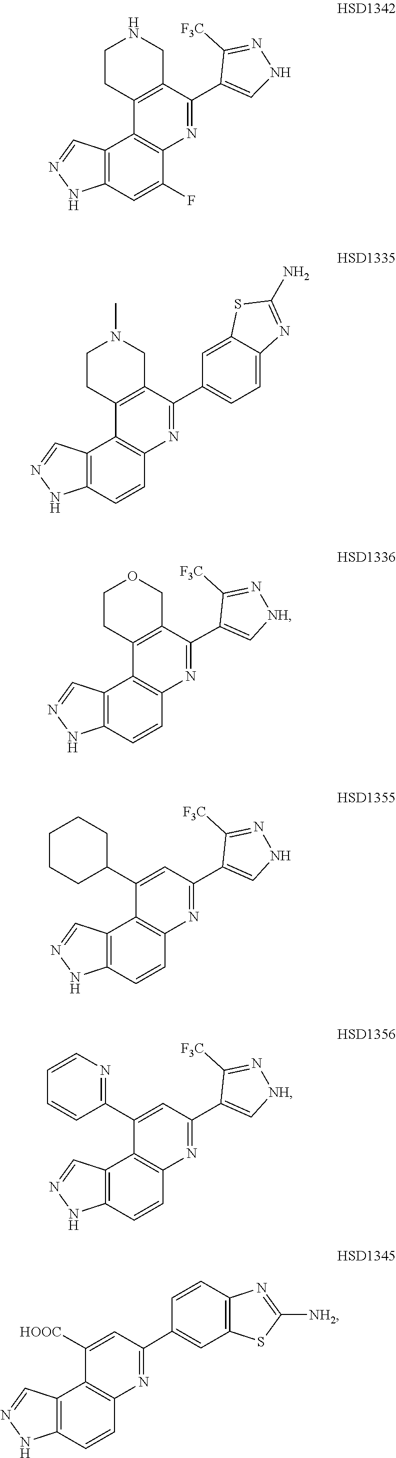 [SMILES: Fc1cc2[nH]ncc2c2c3c(c(-c4c[nH]nc4C(F)(F)F)nc12)CNCC3 ; CN1CCc2c(c(-c3ccc4nc(N)sc4c3)nc3ccc4[nH]ncc4c23)C1 ; FC(F)(F)c1n[nH]cc1-c1nc2ccc3[nH]ncc3c2c2c1COCC2 ; FC(F)(F)c1n[nH]cc1-c1cc(C2CCCCC2)c2c(ccc3[nH]ncc32)n1 ; FC(F)(F)c1n[nH]cc1-c1cc(-c2ccccn2)c2c(ccc3[nH]ncc32)n1 ; Nc1nc2ccc(-c3cc(C(=O)O)c4c(ccc5[nH]ncc54)n3)cc2s1]

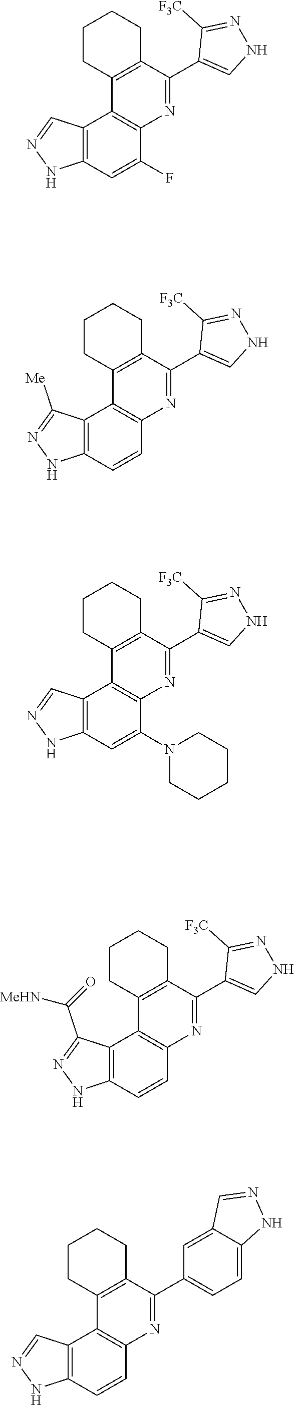 [SMILES: Fc1cc2[nH]ncc2c2c3c(c(-c4c[nH]nc4C(F)(F)F)nc12)CCCC3 ; Cc1n[nH]c2ccc3nc(-c4c[nH]nc4C(F)(F)F)c4c(c3c12)CCCC4 ; FC(F)(F)c1n[nH]cc1-c1nc2c(N3CCCCC3)cc3[nH]ncc3c2c2c1CCCC2 ; CNC(=O)c1n[nH]c2ccc3nc(-c4c[nH]nc4C(F)(F)F)c4c(c3c12)CCCC4 ; c1cc2[nH]ncc2cc1-c1nc2ccc3[nH]ncc3c2c2c1CCCC2]

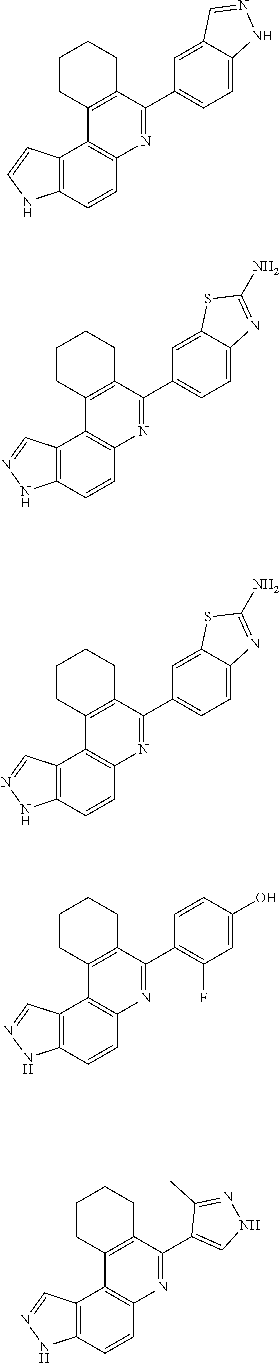 [SMILES: c1cc2c(ccc3nc(-c4ccc5[nH]ncc5c4)c4c(c32)CCCC4)[nH]1 ; Nc1nc2ccc(-c3nc4ccc5[nH]ncc5c4c4c3CCCC4)cc2s1 ; Nc1nc2ccc(-c3nc4ccc5[nH]ncc5c4c4c3CCCC4)cc2s1 ; Oc1ccc(-c2nc3ccc4[nH]ncc4c3c3c2CCCC3)c(F)c1 ; Cc1n[nH]cc1-c1nc2ccc3[nH]ncc3c2c2c1CCCC2]

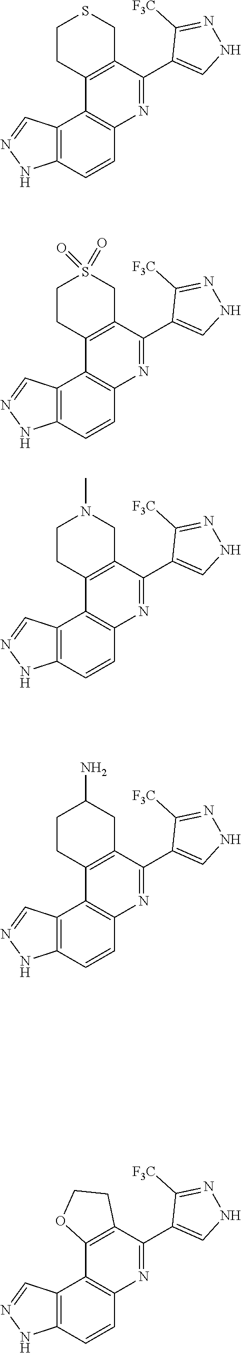

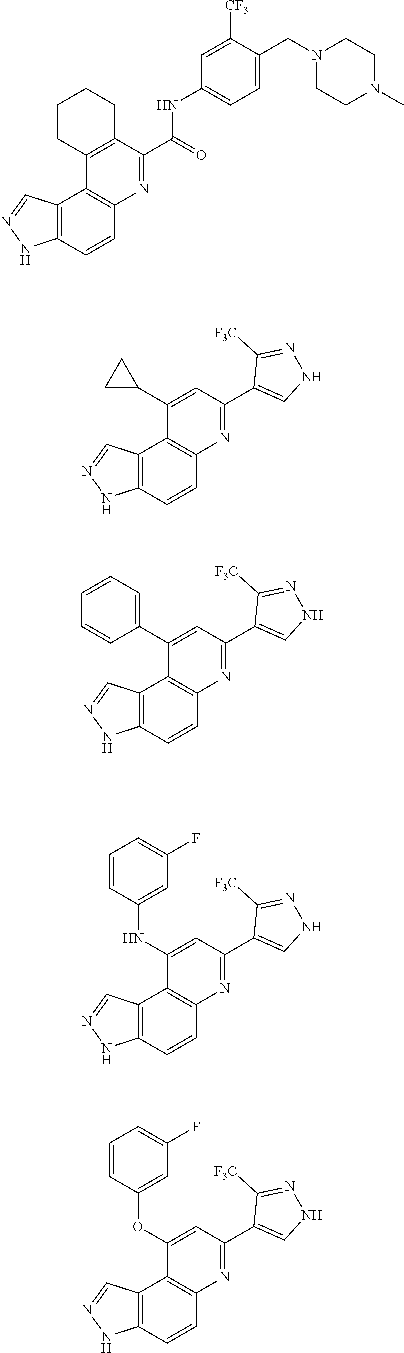 [SMILES: CN1CCN(Cc2ccc(NC(=O)c3nc4ccc5[nH]ncc5c4c4c3CCCC4)cc2C(F)(F)F)CC1 ; FC(F)(F)c1n[nH]cc1-c1cc(C2CC2)c2c(ccc3[nH]ncc32)n1 ; FC(F)(F)c1n[nH]cc1-c1cc(-c2ccccc2)c2c(ccc3[nH]ncc32)n1 ; Fc1cccc(Nc2cc(-c3c[nH]nc3C(F)(F)F)nc3ccc4[nH]ncc4c23)c1 ; Fc1cccc(Oc2cc(-c3c[nH]nc3C(F)(F)F)nc3ccc4[nH]ncc4c23)c1]

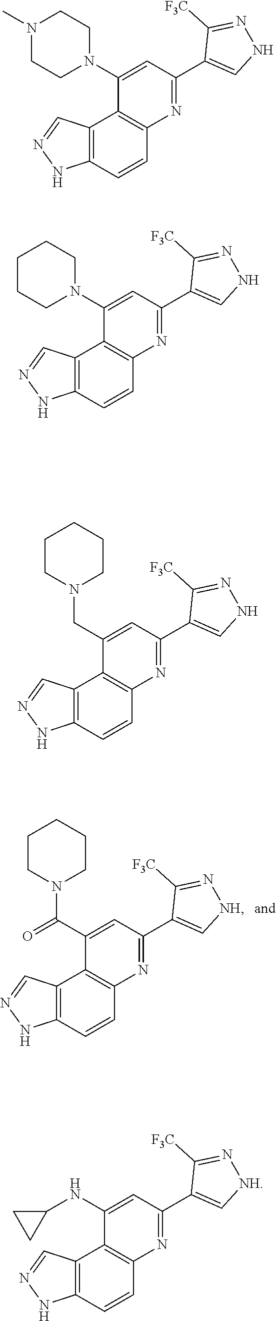 [SMILES: CN1CCN(c2cc(-c3c[nH]nc3C(F)(F)F)nc3ccc4[nH]ncc4c23)CC1 ; FC(F)(F)c1n[nH]cc1-c1cc(N2CCCCC2)c2c(ccc3[nH]ncc32)n1 ; FC(F)(F)c1n[nH]cc1-c1cc(CN2CCCCC2)c2c(ccc3[nH]ncc32)n1 ; O=C(c1cc(-c2c[nH]nc2C(F)(F)F)nc2ccc3[nH]ncc3c12)N1CCCCC1 ; FC(F)(F)c1n[nH]cc1-c1cc(NC2CC2)c2c(ccc3[nH]ncc32)n1]

In some illustrative embodiments, the present invention relates to a compound having a formula (I), wherein the compound is

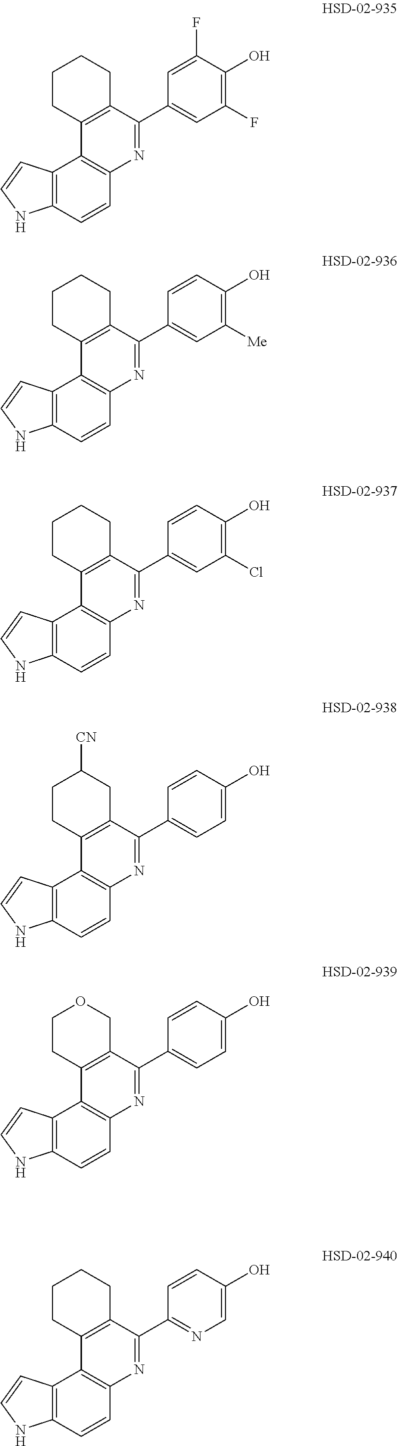 [SMILES: Oc1c(F)cc(-c2nc3ccc4[nH]ccc4c3c3c2CCCC3)cc1F ; Cc1cc(-c2nc3ccc4[nH]ccc4c3c3c2CCCC3)ccc1O ; Oc1ccc(-c2nc3ccc4[nH]ccc4c3c3c2CCCC3)cc1Cl ; N#CC1CCc2c(c(-c3ccc(O)cc3)nc3ccc4[nH]ccc4c23)C1 ; Oc1ccc(-c2nc3ccc4[nH]ccc4c3c3c2COCC3)cc1 ; Oc1ccc(-c2nc3ccc4[nH]ccc4c3c3c2CCCC3)nc1]

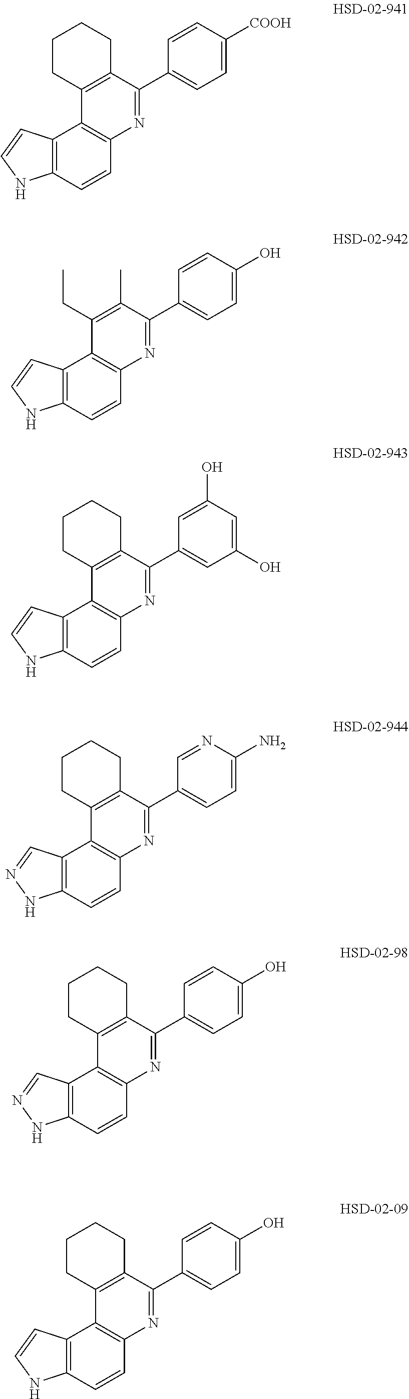 [SMILES: O=C(O)c1ccc(-c2nc3ccc4[nH]ccc4c3c3c2CCCC3)cc1 ; CCc1c(C)c(-c2ccc(O)cc2)nc2ccc3[nH]ccc3c12 ; Oc1cc(O)cc(-c2nc3ccc4[nH]ccc4c3c3c2CCCC3)c1 ; Nc1ccc(-c2nc3ccc4[nH]ncc4c3c3c2CCCC3)cn1 ; Oc1ccc(-c2nc3ccc4[nH]ncc4c3c3c2CCCC3)cc1 ; Oc1ccc(-c2nc3ccc4[nH]ccc4c3c3c2CCCC3)cc1]

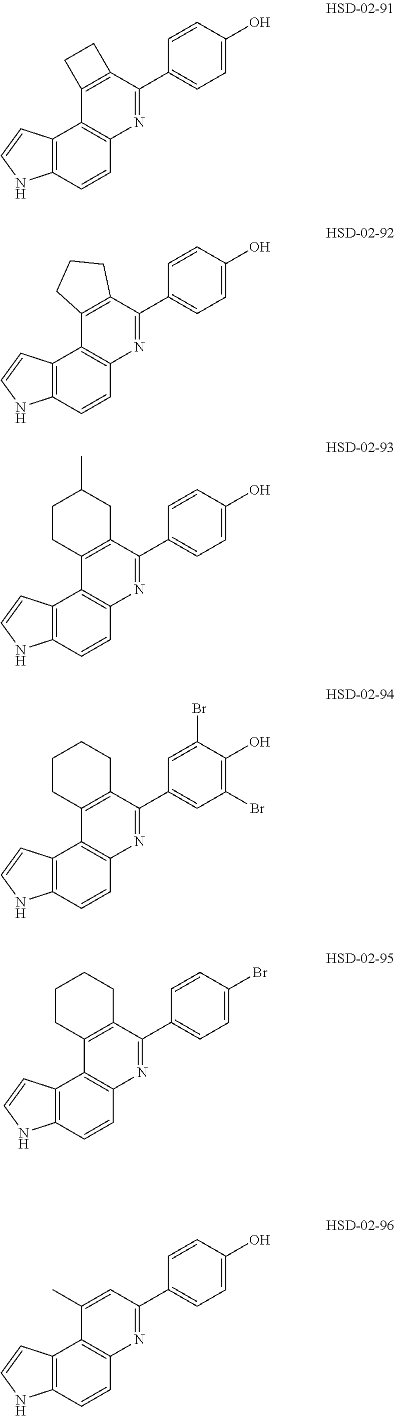

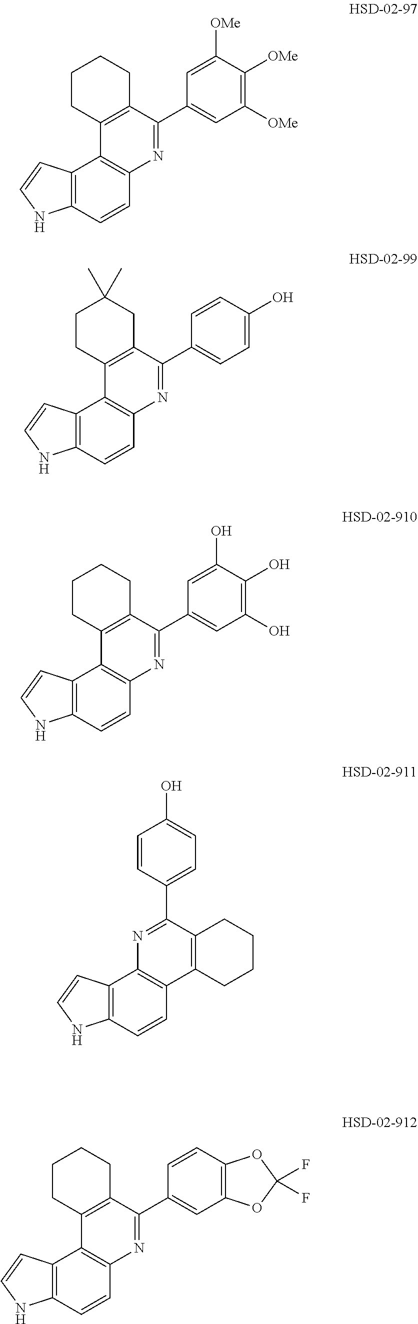 [SMILES: COc1cc(-c2nc3ccc4[nH]ccc4c3c3c2CCCC3)cc(OC)c1OC ; CC1(C)CCc2c(c(-c3ccc(O)cc3)nc3ccc4[nH]ccc4c23)C1 ; Oc1cc(-c2nc3ccc4[nH]ccc4c3c3c2CCCC3)cc(O)c1O ; Oc1ccc(-c2nc3c(ccc4[nH]ccc43)c3c2CCCC3)cc1 ; FC1(F)Oc2ccc(-c3nc4ccc5[nH]ccc5c4c4c3CCCC4)cc2O1]

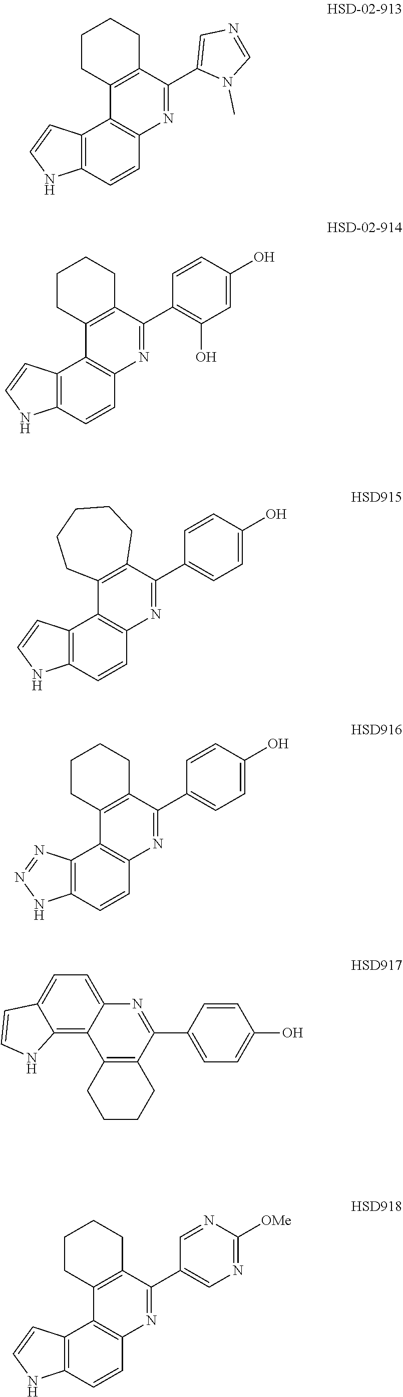 [SMILES: Cn1cncc1-c1nc2ccc3[nH]ccc3c2c2c1CCCC2 ; Oc1ccc(-c2nc3ccc4[nH]ccc4c3c3c2CCCC3)c(O)c1 ; Oc1ccc(-c2nc3ccc4[nH]ccc4c3c3c2CCCCC3)cc1 ; Oc1ccc(-c2nc3ccc4[nH]nnc4c3c3c2CCCC3)cc1 ; Oc1ccc(-c2nc3ccc4cc[nH]c4c3c3c2CCCC3)cc1 ; COc1ncc(-c2nc3ccc4[nH]ccc4c3c3c2CCCC3)cn1]

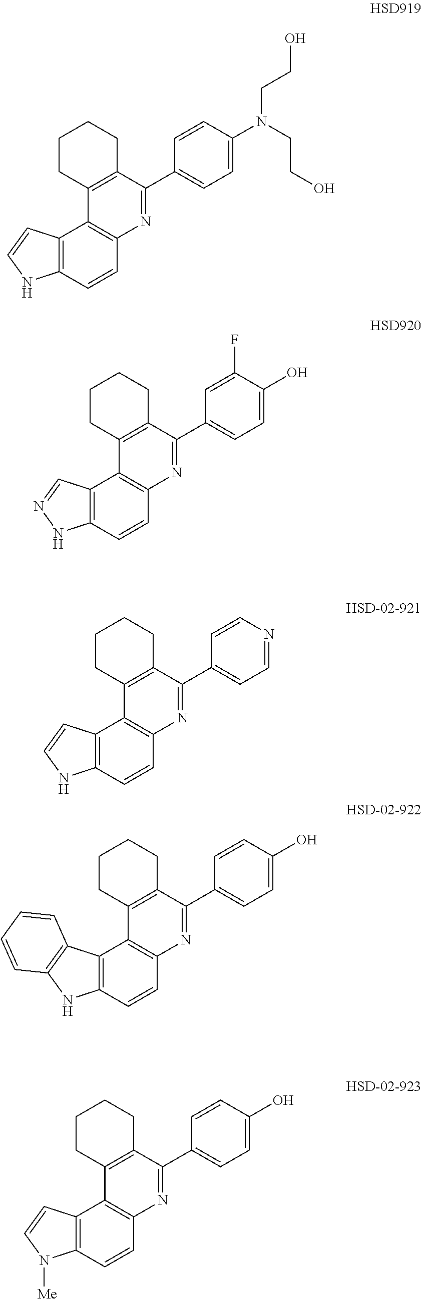 [SMILES: OCCN(CCO)c1ccc(-c2nc3ccc4[nH]ccc4c3c3c2CCCC3)cc1 ; Oc1ccc(-c2nc3ccc4[nH]ncc4c3c3c2CCCC3)cc1F ; c1cc(-c2nc3ccc4[nH]ccc4c3c3c2CCCC3)ccn1 ; Oc1ccc(-c2nc3ccc4[nH]c5ccccc5c4c3c3c2CCCC3)cc1 ; Cn1ccc2c3c4c(c(-c5ccc(O)cc5)nc3ccc21)CCCC4]

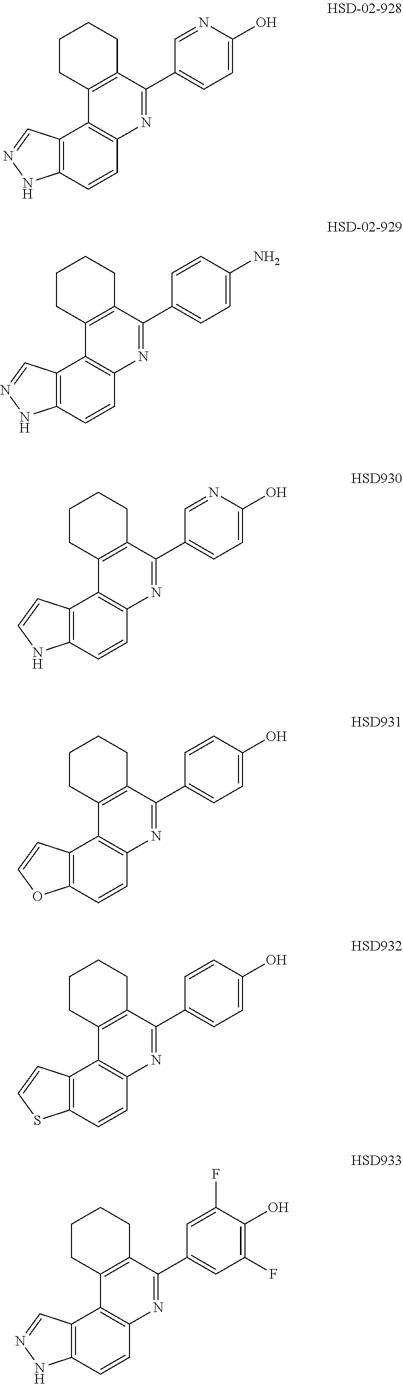 [SMILES: Oc1ccc(-c2nc3ccc4[nH]ncc4c3c3c2CCCC3)cn1 ; Nc1ccc(-c2nc3ccc4[nH]ncc4c3c3c2CCCC3)cc1 ; Oc1ccc(-c2nc3ccc4[nH]ccc4c3c3c2CCCC3)cn1 ; Oc1ccc(-c2nc3ccc4occc4c3c3c2CCCC3)cc1 ; Oc1ccc(-c2nc3ccc4sccc4c3c3c2CCCC3)cc1 ; Oc1c(F)cc(-c2nc3ccc4[nH]ncc4c3c3c2CCCC3)cc1F]

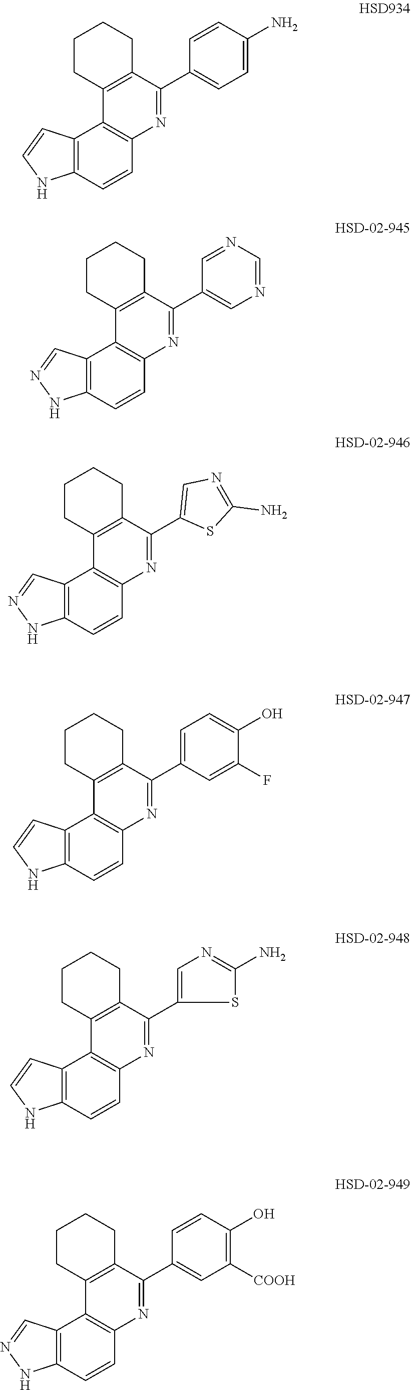 [SMILES: Nc1ccc(-c2nc3ccc4[nH]ccc4c3c3c2CCCC3)cc1 ; c1ncc(-c2nc3ccc4[nH]ncc4c3c3c2CCCC3)cn1 ; Nc1ncc(-c2nc3ccc4[nH]ncc4c3c3c2CCCC3)s1 ; Oc1ccc(-c2nc3ccc4[nH]ccc4c3c3c2CCCC3)cc1F ; Nc1ncc(-c2nc3ccc4[nH]ccc4c3c3c2CCCC3)s1 ; O=C(O)c1cc(-c2nc3ccc4[nH]ncc4c3c3c2CCCC3)ccc1O]

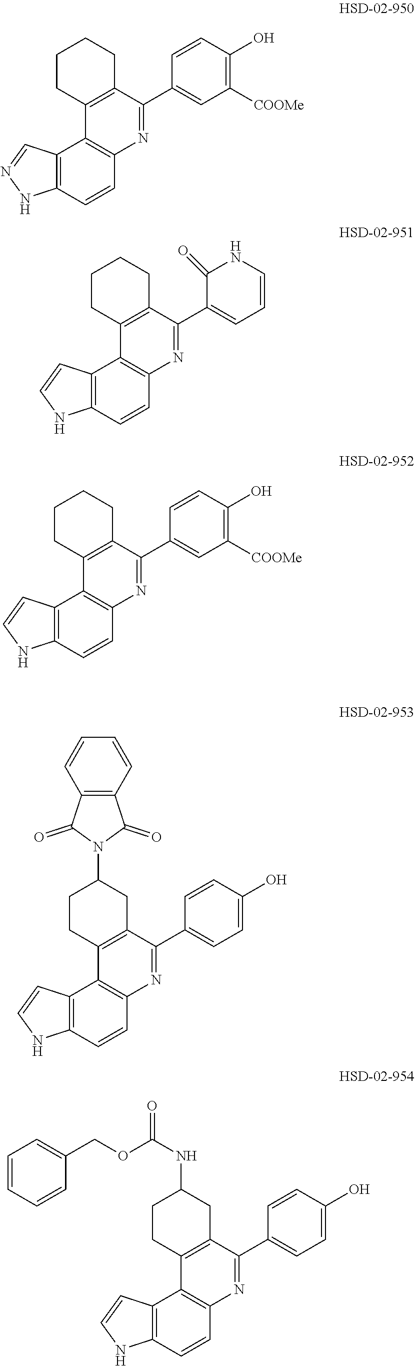 [SMILES: COC(=O)c1cc(-c2nc3ccc4[nH]ncc4c3c3c2CCCC3)ccc1O ; O=c1[nH]cccc1-c1nc2ccc3[nH]ccc3c2c2c1CCCC2 ; COC(=O)c1cc(-c2nc3ccc4[nH]ccc4c3c3c2CCCC3)ccc1O ; O=C1c2ccccc2C(=O)N1C1CCc2c(c(-c3ccc(O)cc3)nc3ccc4[nH]ccc4c23)C1 ; O=C(NC1CCc2c(c(-c3ccc(O)cc3)nc3ccc4[nH]ccc4c23)C1)OCc1ccccc1]

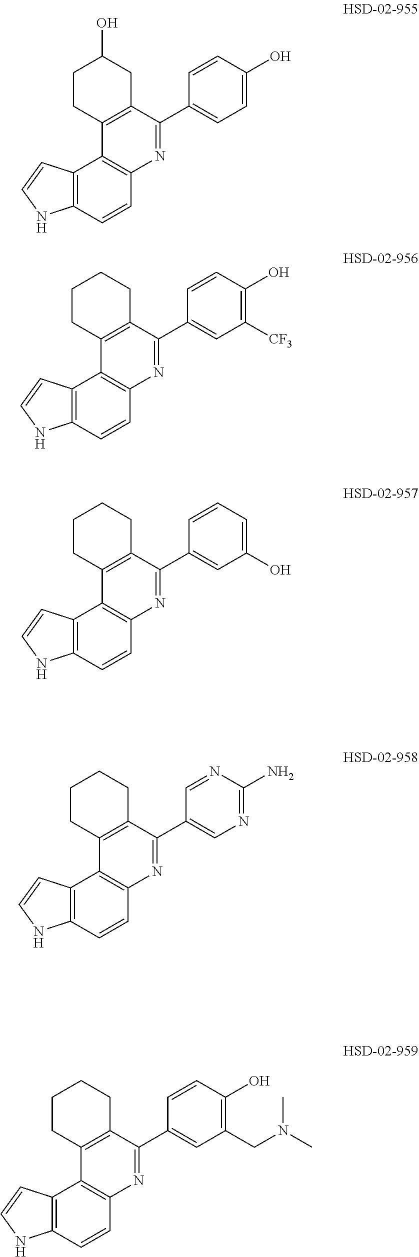 [SMILES: Oc1ccc(-c2nc3ccc4[nH]ccc4c3c3c2CC(O)CC3)cc1 ; Oc1ccc(-c2nc3ccc4[nH]ccc4c3c3c2CCCC3)cc1C(F)(F)F ; Oc1cccc(-c2nc3ccc4[nH]ccc4c3c3c2CCCC3)c1 ; Nc1ncc(-c2nc3ccc4[nH]ccc4c3c3c2CCCC3)cn1 ; CN(C)Cc1cc(-c2nc3ccc4[nH]ccc4c3c3c2CCCC3)ccc1O]

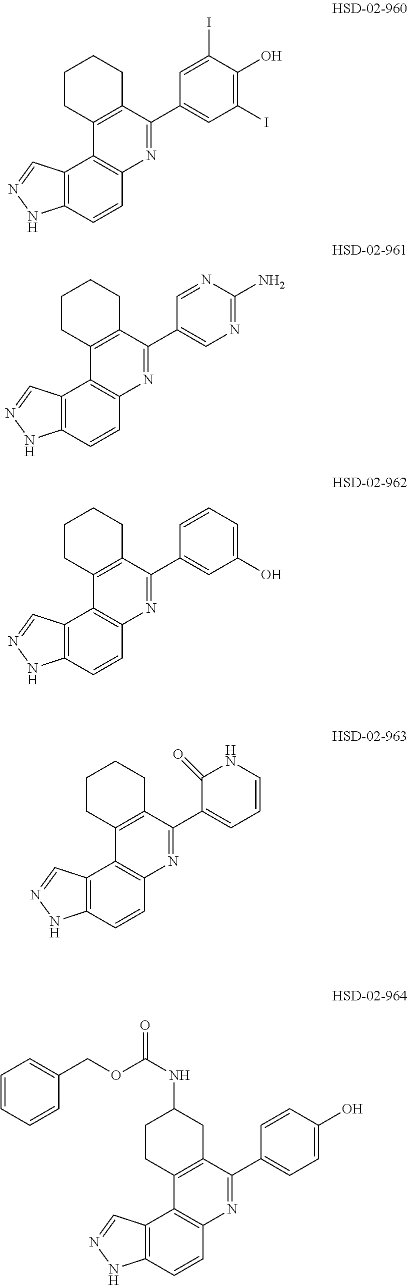 [SMILES: Oc1c(I)cc(-c2nc3ccc4[nH]ncc4c3c3c2CCCC3)cc1I ; Nc1ncc(-c2nc3ccc4[nH]ncc4c3c3c2CCCC3)cn1 ; Oc1cccc(-c2nc3ccc4[nH]ncc4c3c3c2CCCC3)c1 ; O=c1[nH]cccc1-c1nc2ccc3[nH]ncc3c2c2c1CCCC2 ; O=C(NC1CCc2c(c(-c3ccc(O)cc3)nc3ccc4[nH]ncc4c23)C1)OCc1ccccc1]

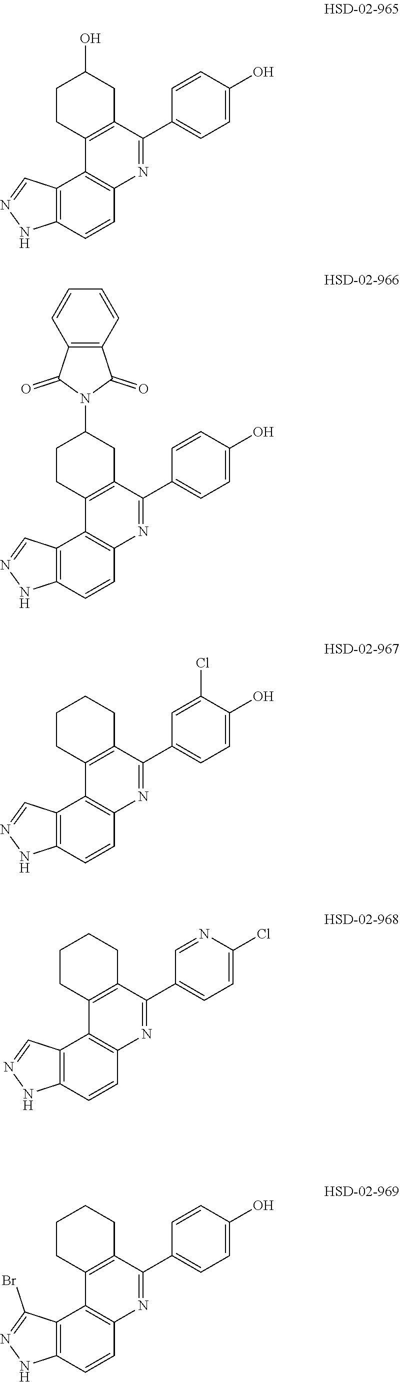 [SMILES: Oc1ccc(-c2nc3ccc4[nH]ncc4c3c3c2CC(O)CC3)cc1 ; O=C1c2ccccc2C(=O)N1C1CCc2c(c(-c3ccc(O)cc3)nc3ccc4[nH]ncc4c23)C1 ; Oc1ccc(-c2nc3ccc4[nH]ncc4c3c3c2CCCC3)cc1Cl ; Clc1ccc(-c2nc3ccc4[nH]ncc4c3c3c2CCCC3)cn1 ; Oc1ccc(-c2nc3ccc4[nH]nc(Br)c4c3c3c2CCCC3)cc1]

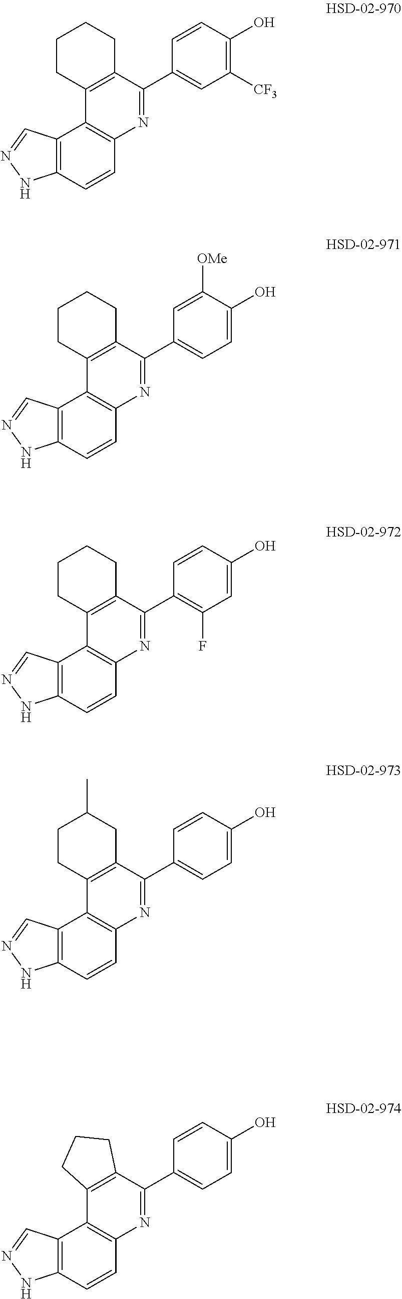 [SMILES: Oc1ccc(-c2nc3ccc4[nH]ncc4c3c3c2CCCC3)cc1C(F)(F)F ; COc1cc(-c2nc3ccc4[nH]ncc4c3c3c2CCCC3)ccc1O ; Oc1ccc(-c2nc3ccc4[nH]ncc4c3c3c2CCCC3)c(F)c1 ; CC1CCc2c(c(-c3ccc(O)cc3)nc3ccc4[nH]ncc4c23)C1 ; Oc1ccc(-c2nc3ccc4[nH]ncc4c3c3c2CCC3)cc1]

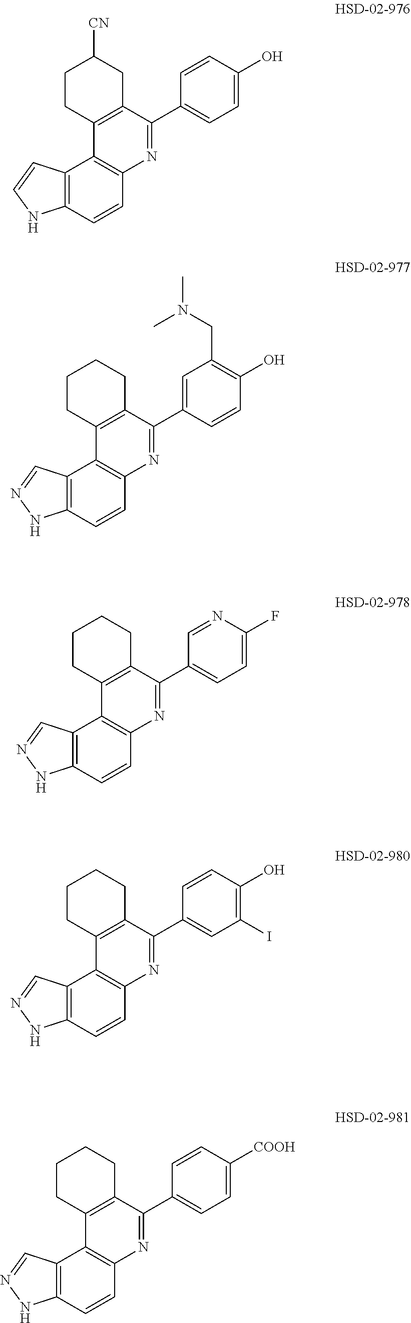 [SMILES: N#CC1CCc2c(c(-c3ccc(O)cc3)nc3ccc4[nH]ccc4c23)C1 ; CN(C)Cc1cc(-c2nc3ccc4[nH]ncc4c3c3c2CCCC3)ccc1O ; Fc1ccc(-c2nc3ccc4[nH]ncc4c3c3c2CCCC3)cn1 ; Oc1ccc(-c2nc3ccc4[nH]ncc4c3c3c2CCCC3)cc1I ; O=C(O)c1ccc(-c2nc3ccc4[nH]ncc4c3c3c2CCCC3)cc1]

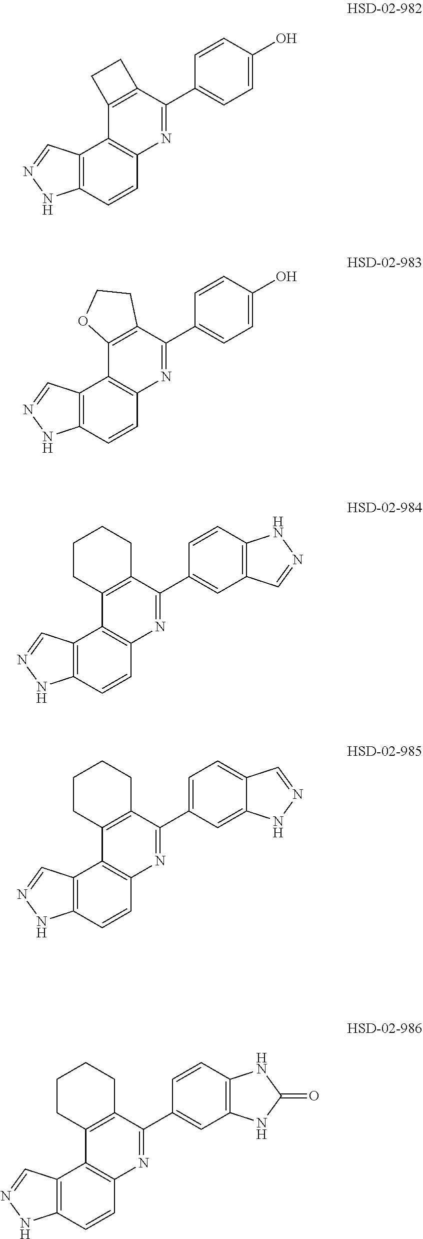 [SMILES: Oc1ccc(-c2nc3ccc4[nH]ncc4c3c3c2CC3)cc1 ; Oc1ccc(-c2nc3ccc4[nH]ncc4c3c3c2CCO3)cc1 ; c1cc2[nH]ncc2cc1-c1nc2ccc3[nH]ncc3c2c2c1CCCC2 ; c1cc2cn[nH]c2cc1-c1nc2ccc3[nH]ncc3c2c2c1CCCC2 ; O=c1[nH]c2ccc(-c3nc4ccc5[nH]ncc5c4c4c3CCCC4)cc2[nH]1]

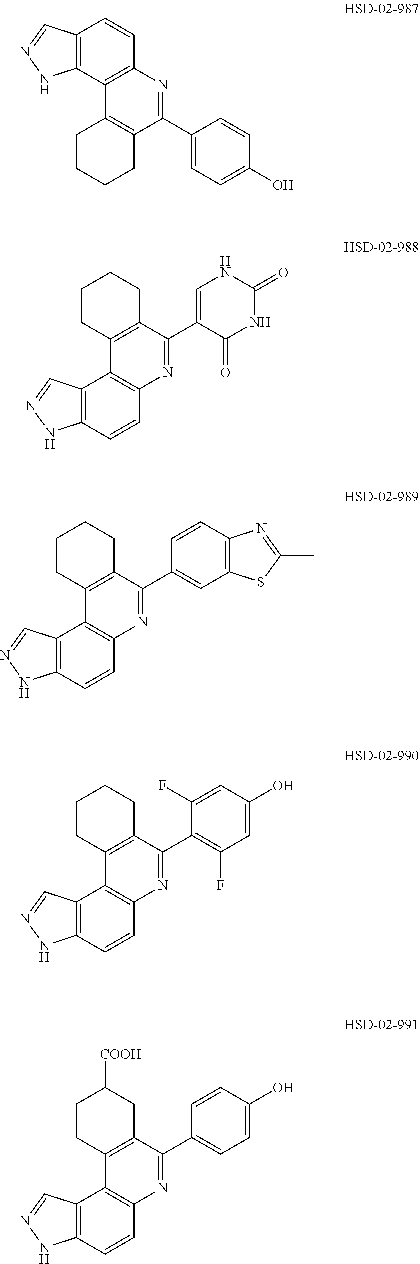 [SMILES: Oc1ccc(-c2nc3ccc4cn[nH]c4c3c3c2CCCC3)cc1 ; O=c1[nH]cc(-c2nc3ccc4[nH]ncc4c3c3c2CCCC3)c(=O)[nH]1 ; Cc1nc2ccc(-c3nc4ccc5[nH]ncc5c4c4c3CCCC4)cc2s1 ; Oc1cc(F)c(-c2nc3ccc4[nH]ncc4c3c3c2CCCC3)c(F)c1 ; O=C(O)C1CCc2c(c(-c3ccc(O)cc3)nc3ccc4[nH]ncc4c23)C1]

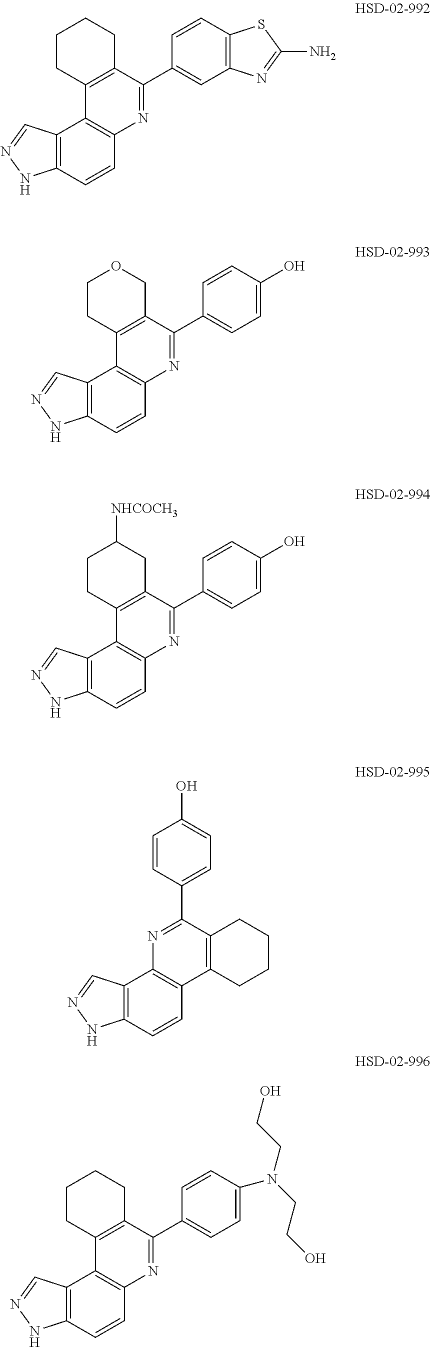

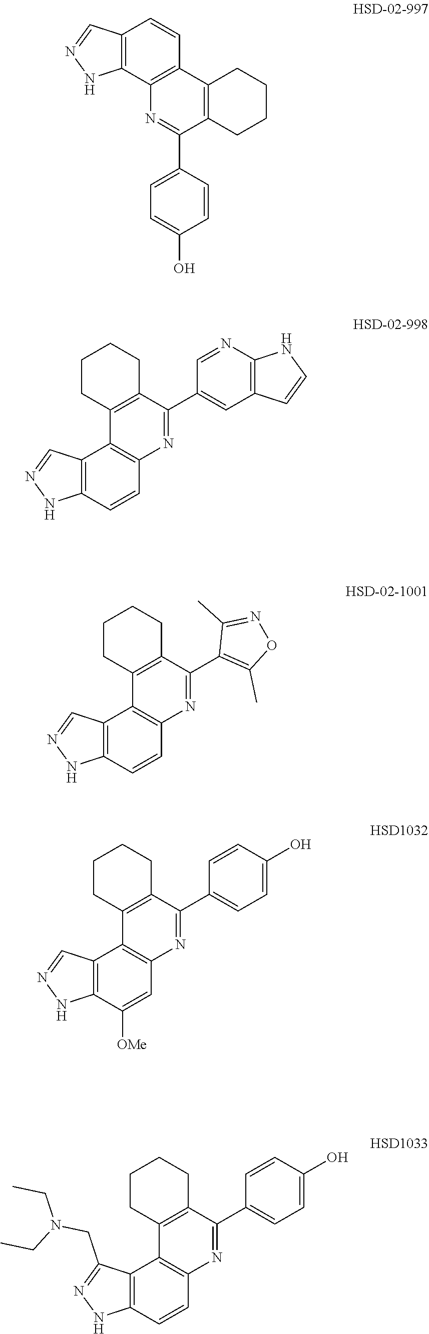 [SMILES: Oc1ccc(-c2nc3c(ccc4cn[nH]c43)c3c2CCCC3)cc1 ; c1cc2cc(-c3nc4ccc5[nH]ncc5c4c4c3CCCC4)cnc2[nH]1 ; Cc1noc(C)c1-c1nc2ccc3[nH]ncc3c2c2c1CCCC2 ; COc1cc2nc(-c3ccc(O)cc3)c3c(c2c2cn[nH]c12)CCCC3 ; CCN(CC)Cc1n[nH]c2ccc3nc(-c4ccc(O)cc4)c4c(c3c12)CCCC4]

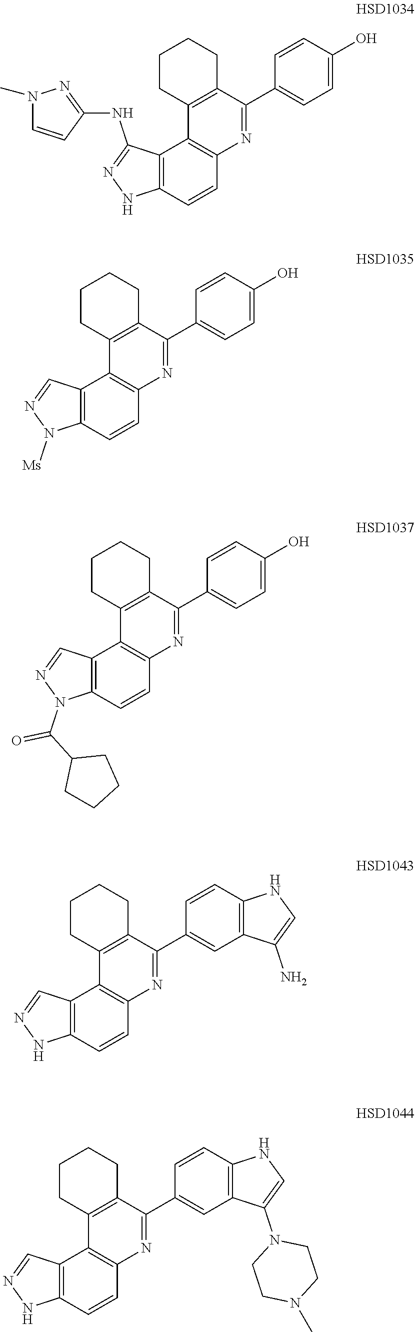 [SMILES: Cn1ccc(Nc2n[nH]c3ccc4nc(-c5ccc(O)cc5)c5c(c4c23)CCCC5)n1 ; CS(=O)(=O)n1ncc2c3c4c(c(-c5ccc(O)cc5)nc3ccc21)CCCC4 ; O=C(C1CCCC1)n1ncc2c3c4c(c(-c5ccc(O)cc5)nc3ccc21)CCCC4 ; Nc1c[nH]c2ccc(-c3nc4ccc5[nH]ncc5c4c4c3CCCC4)cc12 ; CN1CCN(c2c[nH]c3ccc(-c4nc5ccc6[nH]ncc6c5c5c4CCCC5)cc23)CC1]

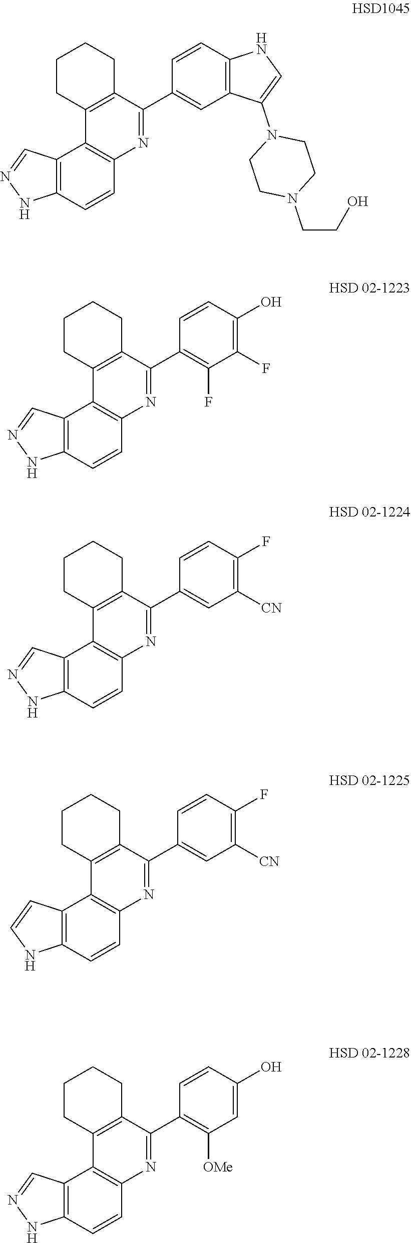 [SMILES: OCCN1CCN(c2c[nH]c3ccc(-c4nc5ccc6[nH]ncc6c5c5c4CCCC5)cc23)CC1 ; Oc1ccc(-c2nc3ccc4[nH]ncc4c3c3c2CCCC3)c(F)c1F ; N#Cc1cc(-c2nc3ccc4[nH]ncc4c3c3c2CCCC3)ccc1F ; N#Cc1cc(-c2nc3ccc4[nH]ccc4c3c3c2CCCC3)ccc1F ; COc1cc(O)ccc1-c1nc2ccc3[nH]ncc3c2c2c1CCCC2]

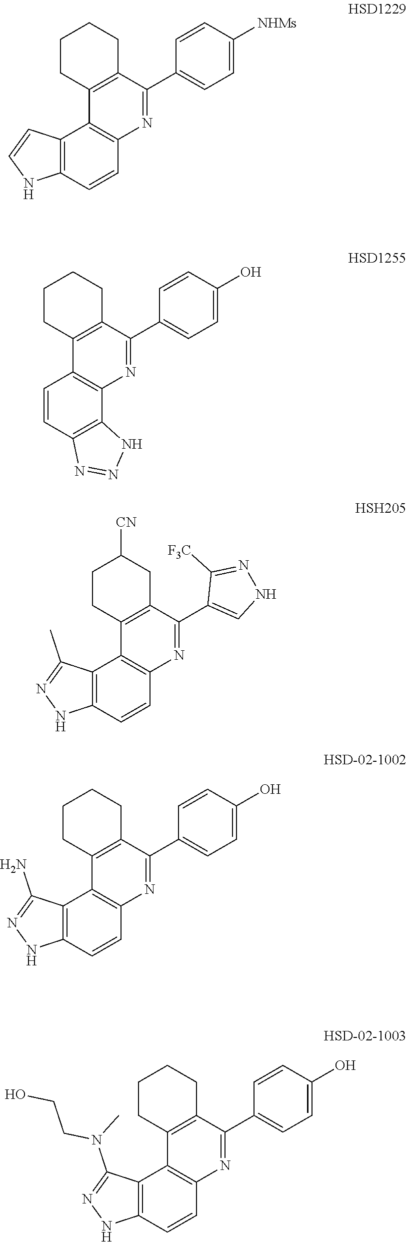

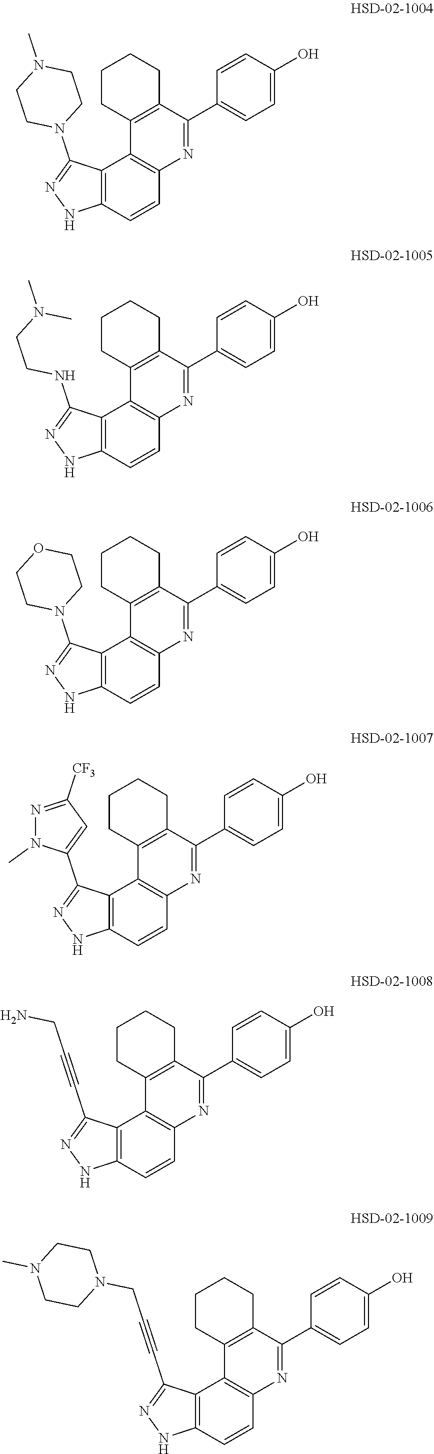 [SMILES: CN1CCN(c2n[nH]c3ccc4nc(-c5ccc(O)cc5)c5c(c4c23)CCCC5)CC1 ; CN(C)CCNc1n[nH]c2ccc3nc(-c4ccc(O)cc4)c4c(c3c12)CCCC4 ; Oc1ccc(-c2nc3ccc4[nH]nc(N5CCOCC5)c4c3c3c2CCCC3)cc1 ; Cn1nc(C(F)(F)F)cc1-c1n[nH]c2ccc3nc(-c4ccc(O)cc4)c4c(c3c12)CCCC4 ; NCC#Cc1n[nH]c2ccc3nc(-c4ccc(O)cc4)c4c(c3c12)CCCC4 ; CN1CCN(CC#Cc2n[nH]c3ccc4nc(-c5ccc(O)cc5)c5c(c4c23)CCCC5)CC1]

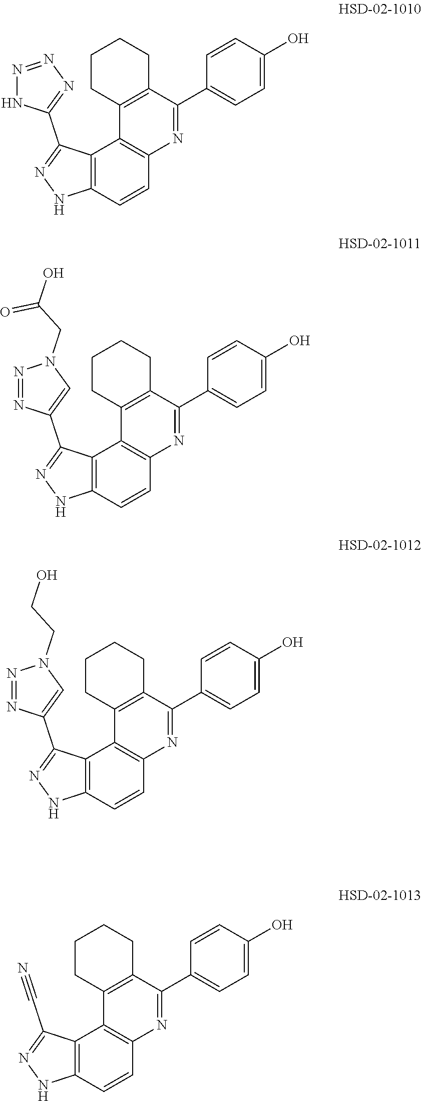 [SMILES: Oc1ccc(-c2nc3ccc4[nH]nc(-c5nnn[nH]5)c4c3c3c2CCCC3)cc1 ; O=C(O)Cn1cc(-c2n[nH]c3ccc4nc(-c5ccc(O)cc5)c5c(c4c23)CCCC5)nn1 ; OCCn1cc(-c2n[nH]c3ccc4nc(-c5ccc(O)cc5)c5c(c4c23)CCCC5)nn1 ; N#Cc1n[nH]c2ccc3nc(-c4ccc(O)cc4)c4c(c3c12)CCCC4]

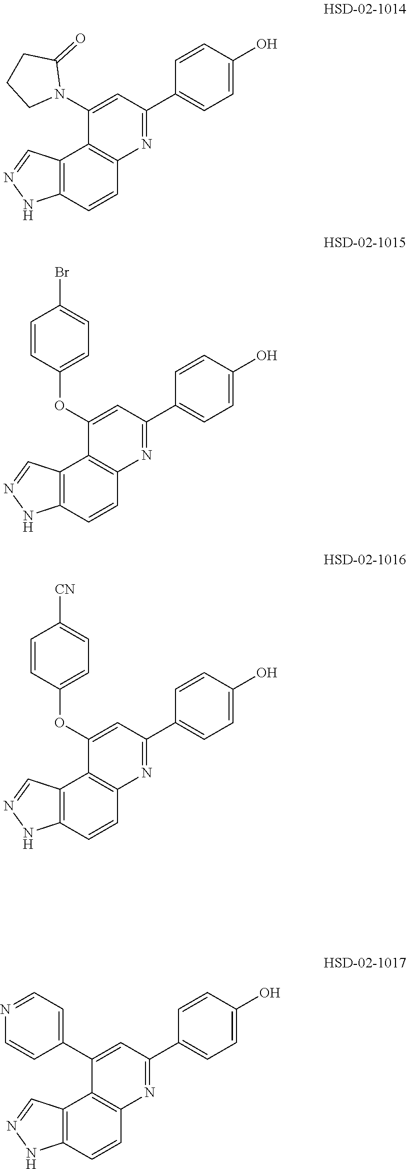 [SMILES: O=C1CCCN1c1cc(-c2ccc(O)cc2)nc2ccc3[nH]ncc3c12 ; Oc1ccc(-c2cc(Oc3ccc(Br)cc3)c3c(ccc4[nH]ncc43)n2)cc1 ; N#Cc1ccc(Oc2cc(-c3ccc(O)cc3)nc3ccc4[nH]ncc4c23)cc1 ; Oc1ccc(-c2cc(-c3ccncc3)c3c(ccc4[nH]ncc43)n2)cc1]

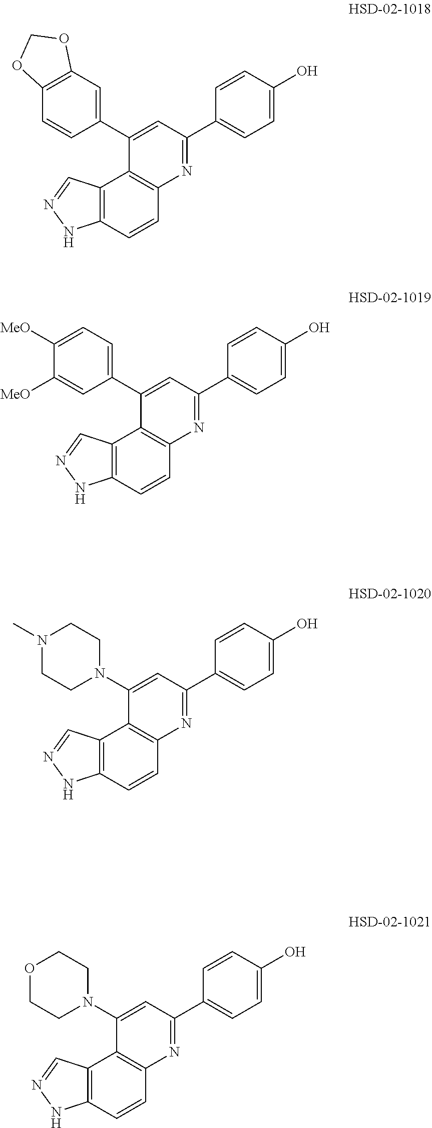 [SMILES: Oc1ccc(-c2cc(-c3ccc4c(c3)OCO4)c3c(ccc4[nH]ncc43)n2)cc1 ; COc1ccc(-c2cc(-c3ccc(O)cc3)nc3ccc4[nH]ncc4c23)cc1OC ; CN1CCN(c2cc(-c3ccc(O)cc3)nc3ccc4[nH]ncc4c23)CC1 ; Oc1ccc(-c2cc(N3CCOCC3)c3c(ccc4[nH]ncc43)n2)cc1]

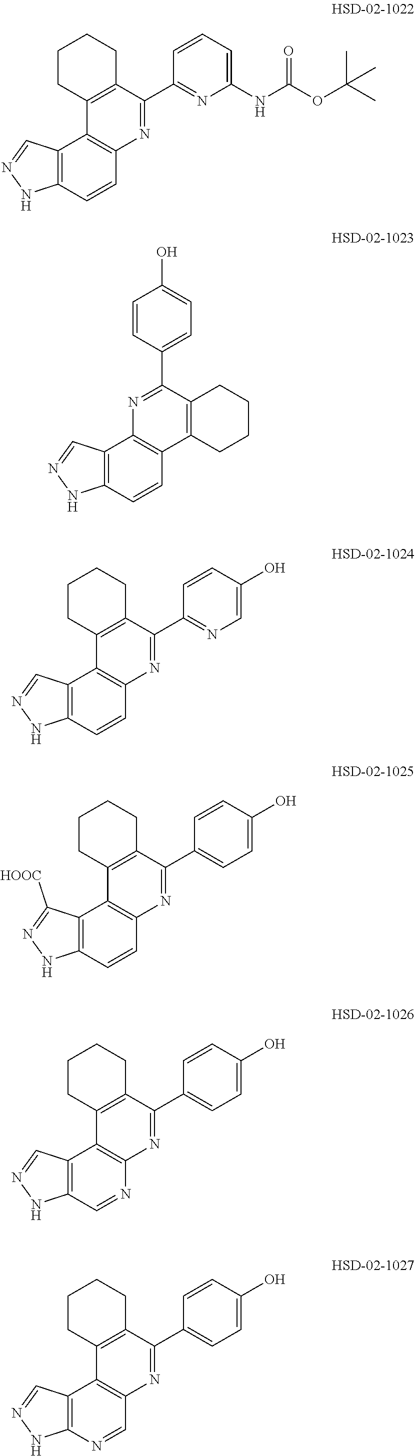 [SMILES: CC(C)(C)OC(=O)Nc1cccc(-c2nc3ccc4[nH]ncc4c3c3c2CCCC3)n1 ; Oc1ccc(-c2nc3c(ccc4[nH]ncc43)c3c2CCCC3)cc1 ; Oc1ccc(-c2nc3ccc4[nH]ncc4c3c3c2CCCC3)nc1 ; O=C(O)c1n[nH]c2ccc3nc(-c4ccc(O)cc4)c4c(c3c12)CCCC4 ; Oc1ccc(-c2nc3ncc4[nH]ncc4c3c3c2CCCC3)cc1 ; Oc1ccc(-c2nc3cnc4[nH]ncc4c3c3c2CCCC3)cc1]

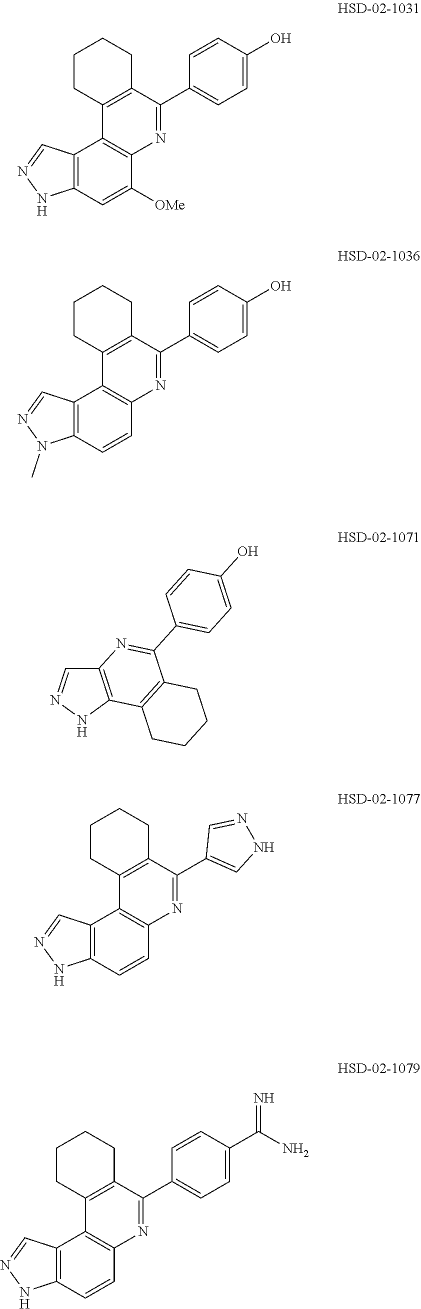 [SMILES: COc1cc2[nH]ncc2c2c3c(c(-c4ccc(O)cc4)nc12)CCCC3 ; Cn1ncc2c3c4c(c(-c5ccc(O)cc5)nc3ccc21)CCCC4 ; Oc1ccc(-c2nc3cn[nH]c3c3c2CCCC3)cc1 ; c1n[nH]cc1-c1nc2ccc3[nH]ncc3c2c2c1CCCC2 ; N=C(N)c1ccc(-c2nc3ccc4[nH]ncc4c3c3c2CCCC3)cc1]

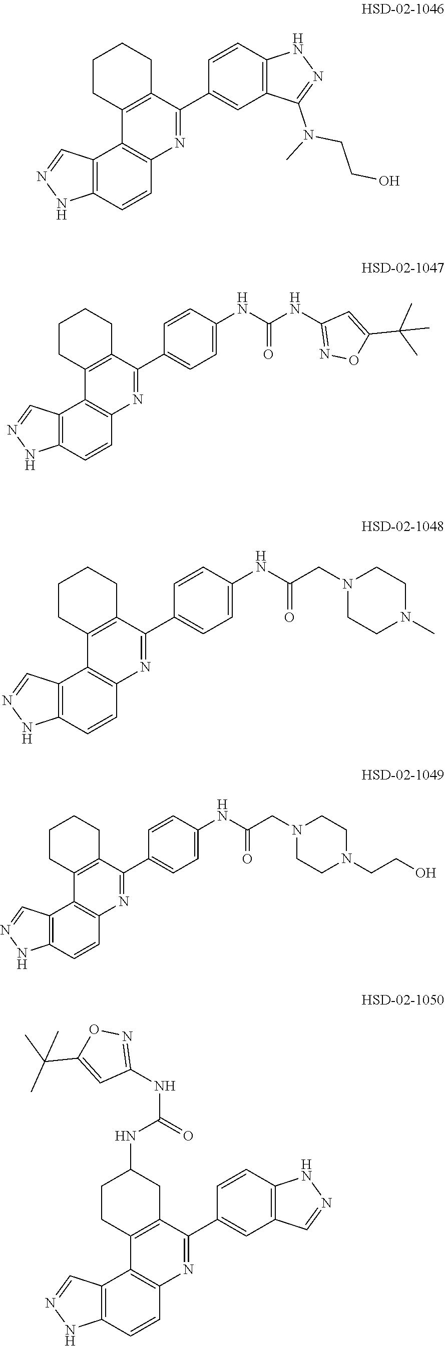

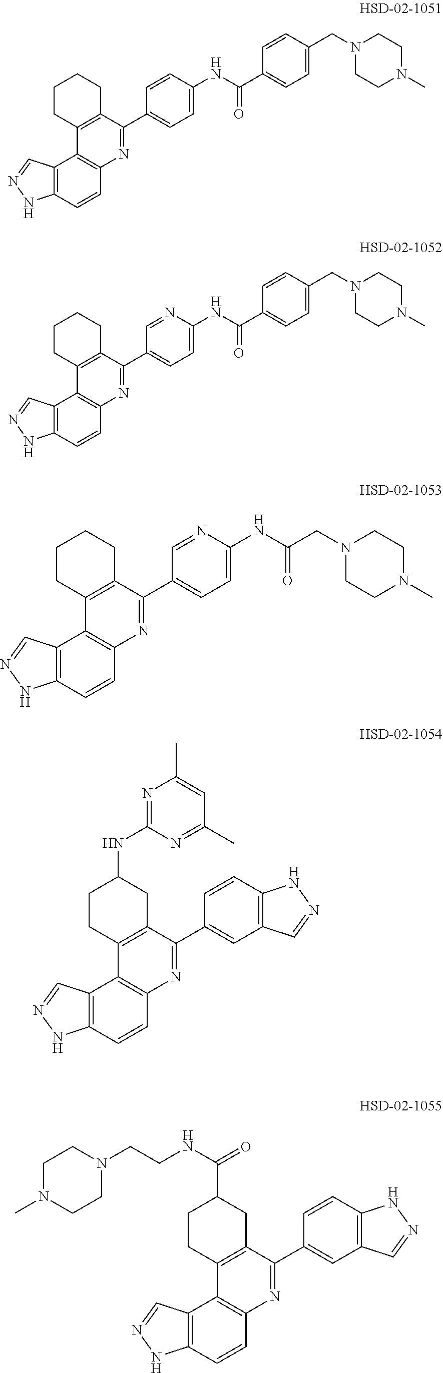

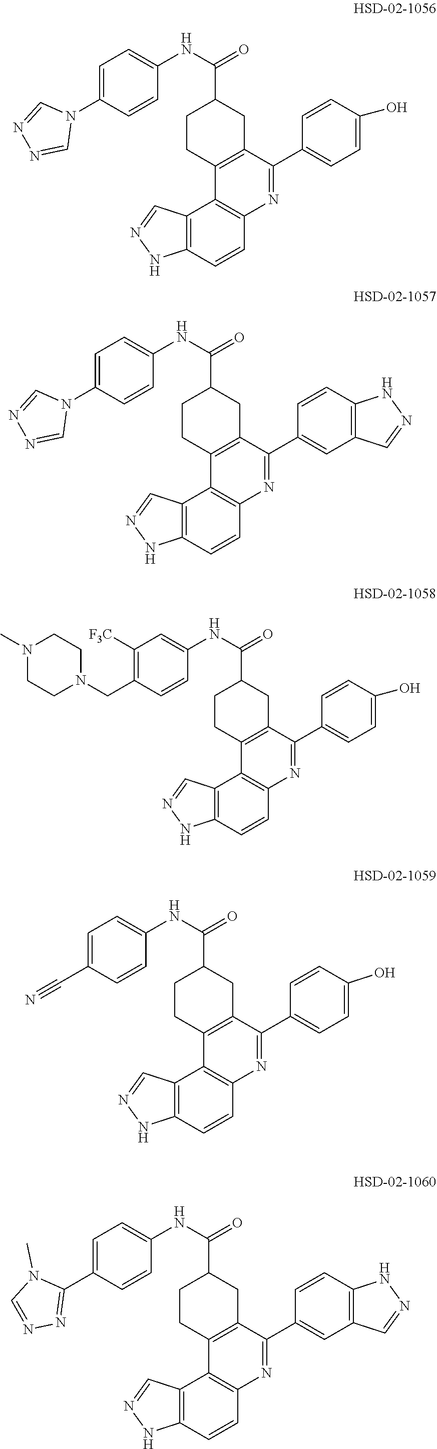 [SMILES: O=C(Nc1ccc(-n2cnnc2)cc1)C1CCc2c(c(-c3ccc(O)cc3)nc3ccc4[nH]ncc4c23)C1 ; O=C(Nc1ccc(-n2cnnc2)cc1)C1CCc2c(c(-c3ccc4[nH]ncc4c3)nc3ccc4[nH]ncc4c23)C1 ; CN1CCN(Cc2ccc(NC(=O)C3CCc4c(c(-c5ccc(O)cc5)nc5ccc6[nH]ncc6c45)C3)cc2C(F)(F)F)CC1 ; N#Cc1ccc(NC(=O)C2CCc3c(c(-c4ccc(O)cc4)nc4ccc5[nH]ncc5c34)C2)cc1 ; Cn1cnnc1-c1ccc(NC(=O)C2CCc3c(c(-c4ccc5[nH]ncc5c4)nc4ccc5[nH]ncc5c34)C2)cc1]

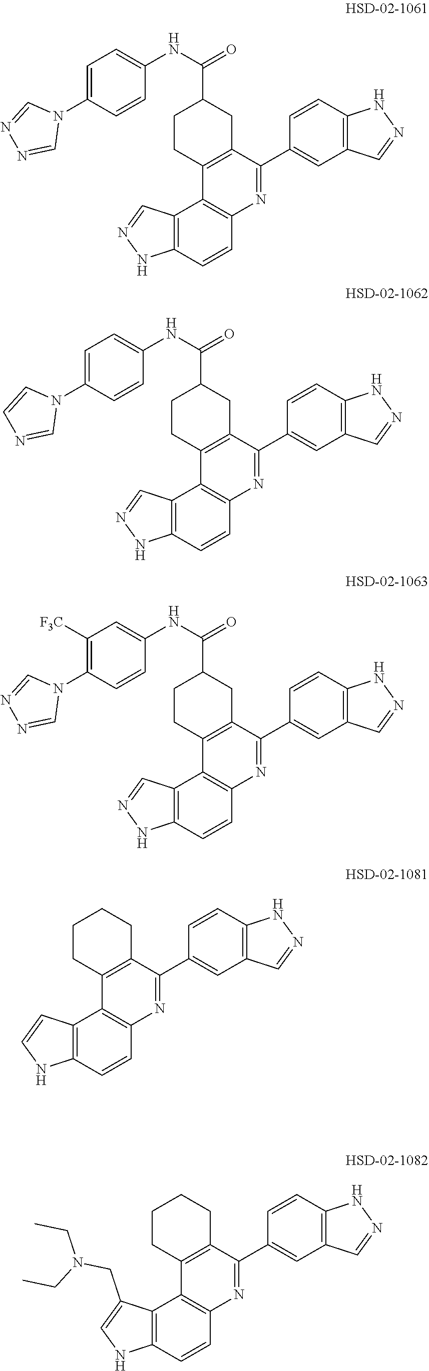 [SMILES: O=C(Nc1ccc(-n2cnnc2)cc1)C1CCc2c(c(-c3ccc4[nH]ncc4c3)nc3ccc4[nH]ncc4c23)C1 ; O=C(Nc1ccc(-n2ccnc2)cc1)C1CCc2c(c(-c3ccc4[nH]ncc4c3)nc3ccc4[nH]ncc4c23)C1 ; O=C(Nc1ccc(-n2cnnc2)c(C(F)(F)F)c1)C1CCc2c(c(-c3ccc4[nH]ncc4c3)nc3ccc4[nH]ncc4c23)C1 ; c1cc2c(ccc3nc(-c4ccc5[nH]ncc5c4)c4c(c32)CCCC4)[nH]1 ; CCN(CC)Cc1c[nH]c2ccc3nc(-c4ccc5[nH]ncc5c4)c4c(c3c12)CCCC4]

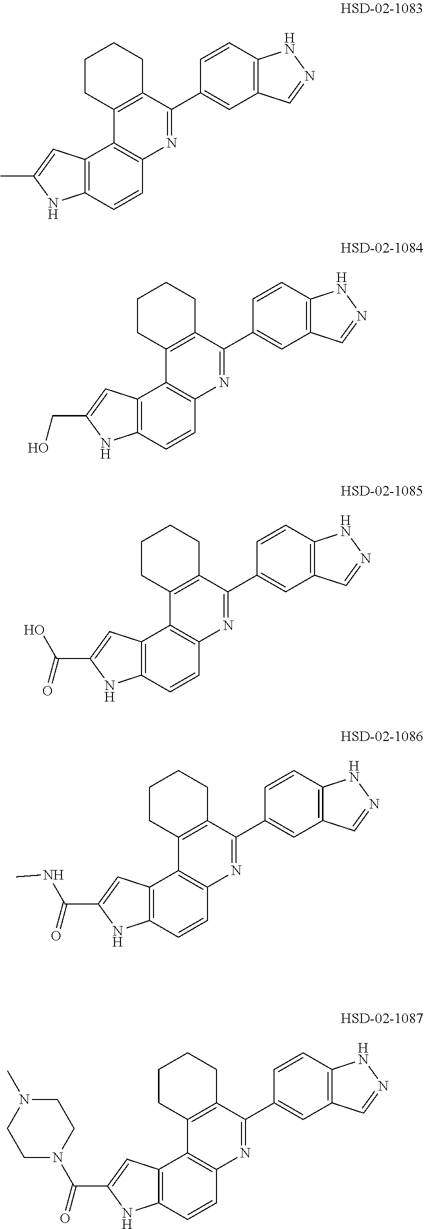 [SMILES: Cc1cc2c(ccc3nc(-c4ccc5[nH]ncc5c4)c4c(c32)CCCC4)[nH]1 ; OCc1cc2c(ccc3nc(-c4ccc5[nH]ncc5c4)c4c(c32)CCCC4)[nH]1 ; O=C(O)c1cc2c(ccc3nc(-c4ccc5[nH]ncc5c4)c4c(c32)CCCC4)[nH]1 ; CNC(=O)c1cc2c(ccc3nc(-c4ccc5[nH]ncc5c4)c4c(c32)CCCC4)[nH]1 ; CN1CCN(C(=O)c2cc3c(ccc4nc(-c5ccc6[nH]ncc6c5)c5c(c43)CCCC5)[nH]2)CC1]

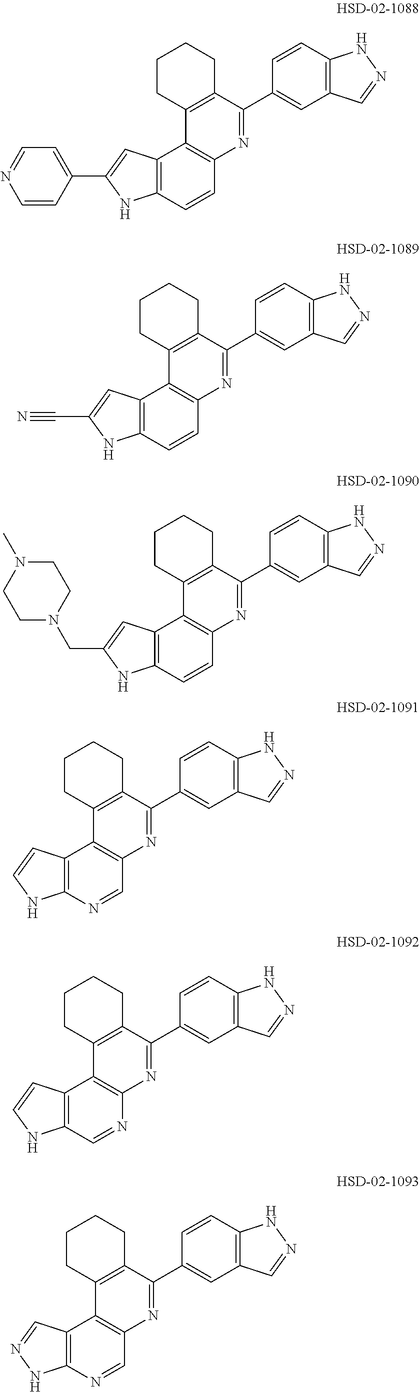 [SMILES: c1cc(-c2cc3c(ccc4nc(-c5ccc6[nH]ncc6c5)c5c(c43)CCCC5)[nH]2)ccn1 ; N#Cc1cc2c(ccc3nc(-c4ccc5[nH]ncc5c4)c4c(c32)CCCC4)[nH]1 ; CN1CCN(Cc2cc3c(ccc4nc(-c5ccc6[nH]ncc6c5)c5c(c43)CCCC5)[nH]2)CC1 ; c1cc2c(ncc3nc(-c4ccc5[nH]ncc5c4)c4c(c32)CCCC4)[nH]1 ; c1cc2c(cnc3nc(-c4ccc5[nH]ncc5c4)c4c(c32)CCCC4)[nH]1 ; c1cc2[nH]ncc2cc1-c1nc2cnc3[nH]ncc3c2c2c1CCCC2]

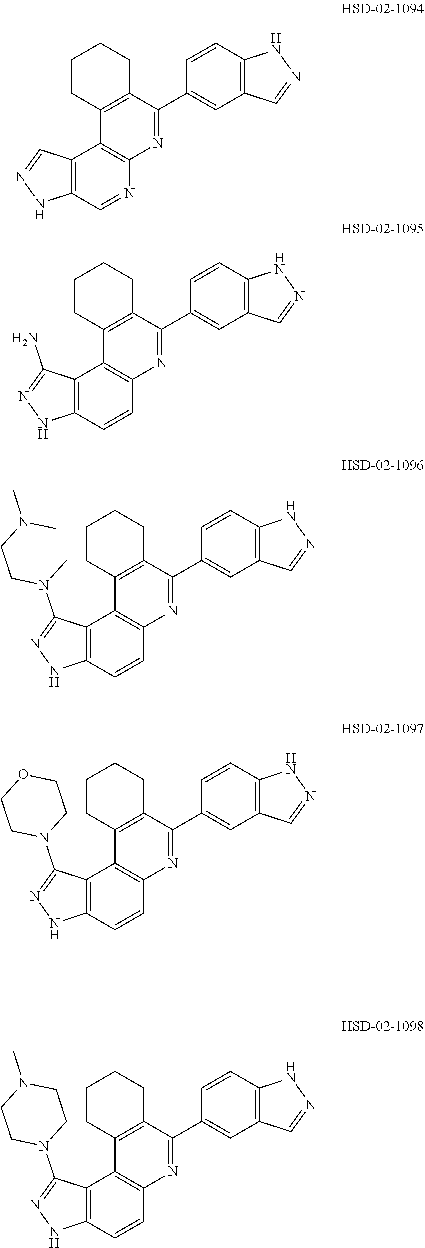 [SMILES: c1cc2[nH]ncc2cc1-c1nc2ncc3[nH]ncc3c2c2c1CCCC2 ; Nc1n[nH]c2ccc3nc(-c4ccc5[nH]ncc5c4)c4c(c3c12)CCCC4 ; CN(C)CCN(C)c1n[nH]c2ccc3nc(-c4ccc5[nH]ncc5c4)c4c(c3c12)CCCC4 ; c1cc2[nH]ncc2cc1-c1nc2ccc3[nH]nc(N4CCOCC4)c3c2c2c1CCCC2 ; CN1CCN(c2n[nH]c3ccc4nc(-c5ccc6[nH]ncc6c5)c5c(c4c23)CCCC5)CC1]

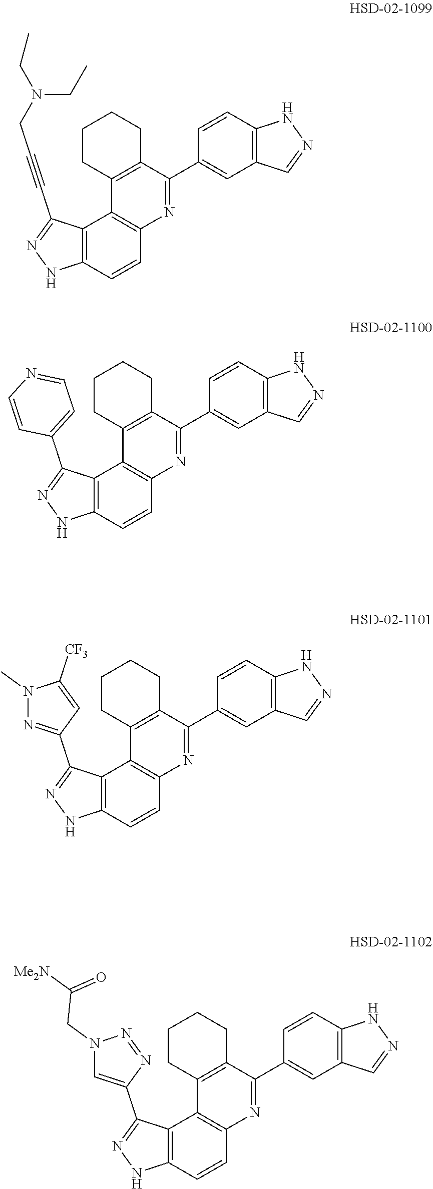 [SMILES: CCN(CC)CC#Cc1n[nH]c2ccc3nc(-c4ccc5[nH]ncc5c4)c4c(c3c12)CCCC4 ; c1cc(-c2n[nH]c3ccc4nc(-c5ccc6[nH]ncc6c5)c5c(c4c23)CCCC5)ccn1 ; Cn1nc(-c2n[nH]c3ccc4nc(-c5ccc6[nH]ncc6c5)c5c(c4c23)CCCC5)cc1C(F)(F)F ; CN(C)C(=O)Cn1cc(-c2n[nH]c3ccc4nc(-c5ccc6[nH]ncc6c5)c5c(c4c23)CCCC5)nn1]

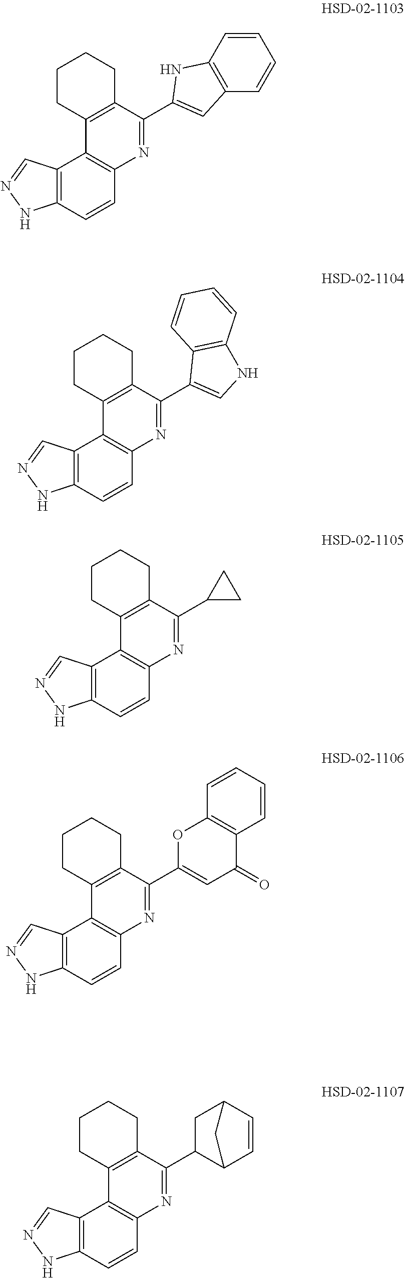 [SMILES: c1ccc2[nH]c(-c3nc4ccc5[nH]ncc5c4c4c3CCCC4)cc2c1 ; c1ccc2c(-c3nc4ccc5[nH]ncc5c4c4c3CCCC4)c[nH]c2c1 ; c1cc2[nH]ncc2c2c3c(c(C4CC4)nc12)CCCC3 ; O=c1cc(-c2nc3ccc4[nH]ncc4c3c3c2CCCC3)oc2ccccc12 ; C1=CC2CC1CC2c1nc2ccc3[nH]ncc3c2c2c1CCCC2]

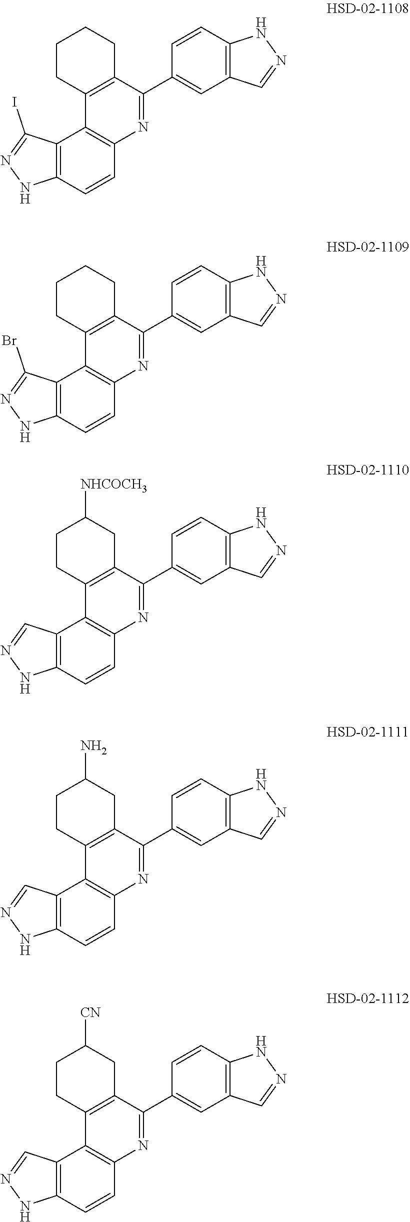

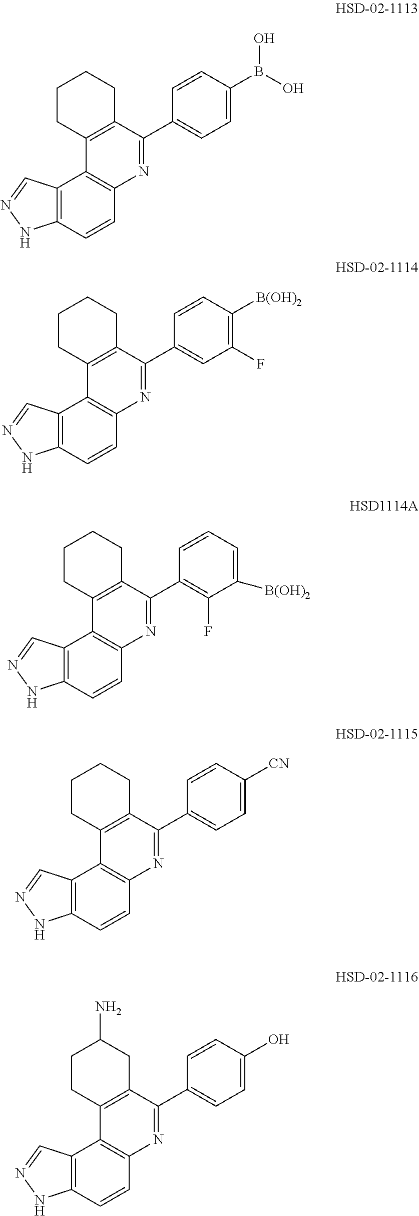 [SMILES: OB(O)c1ccc(-c2nc3ccc4[nH]ncc4c3c3c2CCCC3)cc1 ; OB(O)c1ccc(-c2nc3ccc4[nH]ncc4c3c3c2CCCC3)cc1F ; OB(O)c1cccc(-c2nc3ccc4[nH]ncc4c3c3c2CCCC3)c1F ; N#Cc1ccc(-c2nc3ccc4[nH]ncc4c3c3c2CCCC3)cc1 ; NC1CCc2c(c(-c3ccc(O)cc3)nc3ccc4[nH]ncc4c23)C1]

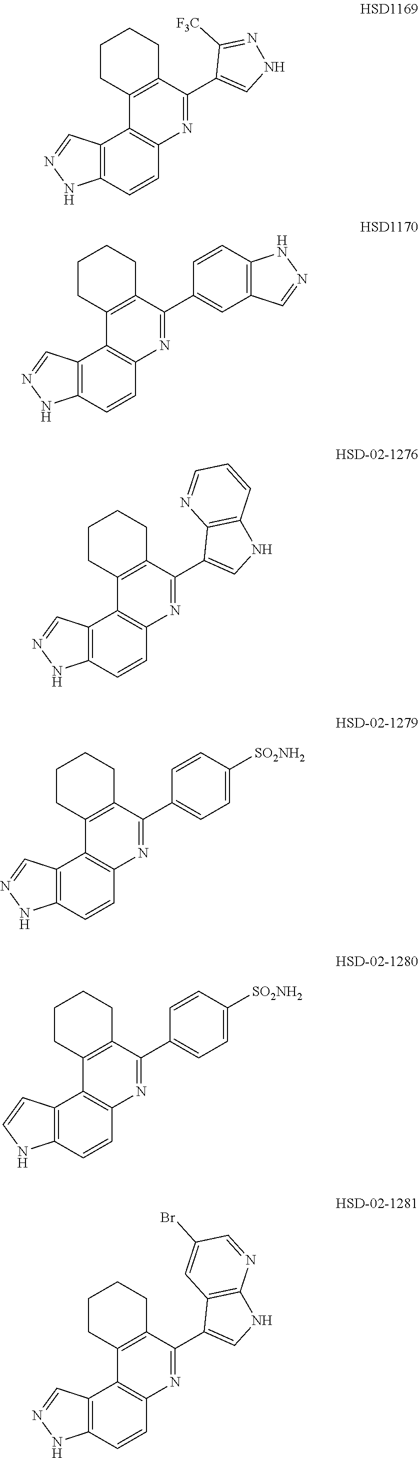 [SMILES: FC(F)(F)c1n[nH]cc1-c1nc2ccc3[nH]ncc3c2c2c1CCCC2 ; c1cc2[nH]ncc2cc1-c1nc2ccc3[nH]ncc3c2c2c1CCCC2 ; c1cnc2c(-c3nc4ccc5[nH]ncc5c4c4c3CCCC4)c[nH]c2c1 ; NS(=O)(=O)c1ccc(-c2nc3ccc4[nH]ncc4c3c3c2CCCC3)cc1 ; NS(=O)(=O)c1ccc(-c2nc3ccc4[nH]ccc4c3c3c2CCCC3)cc1 ; Brc1cnc2[nH]cc(-c3nc4ccc5[nH]ncc5c4c4c3CCCC4)c2c1]

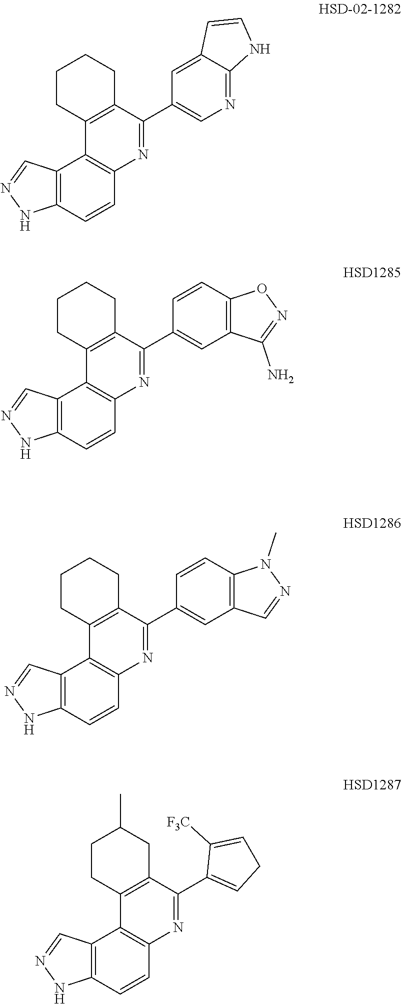 [SMILES: c1cc2cc(-c3nc4ccc5[nH]ncc5c4c4c3CCCC4)cnc2[nH]1 ; Nc1noc2ccc(-c3nc4ccc5[nH]ncc5c4c4c3CCCC4)cc12 ; Cn1ncc2cc(-c3nc4ccc5[nH]ncc5c4c4c3CCCC4)ccc21 ; CC1CCc2c(c(C3=CCC=C3C(F)(F)F)nc3ccc4[nH]ncc4c23)C1]

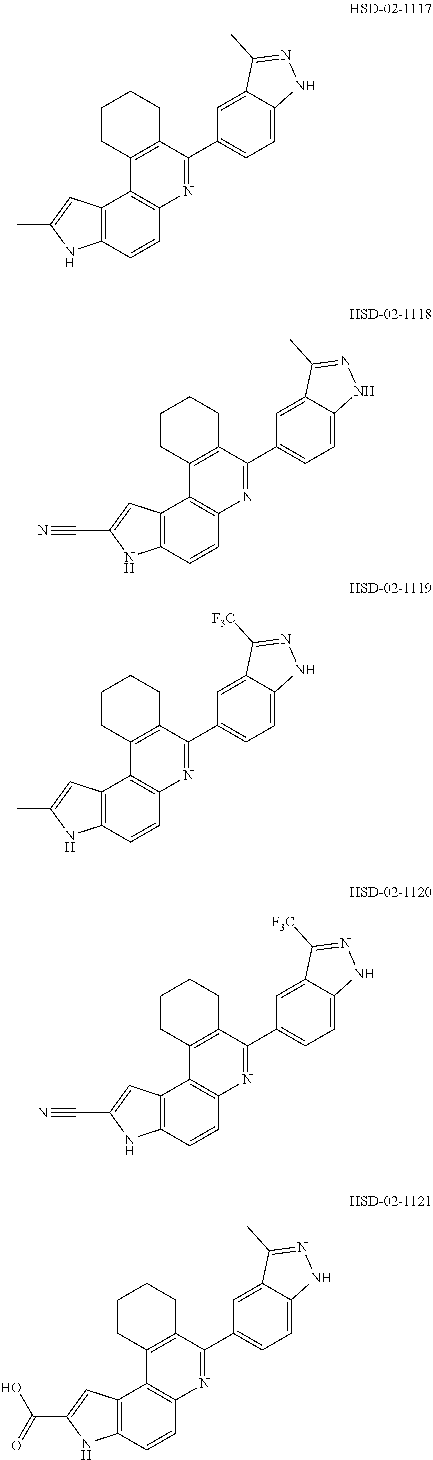 [SMILES: Cc1cc2c(ccc3nc(-c4ccc5[nH]nc(C)c5c4)c4c(c32)CCCC4)[nH]1 ; Cc1n[nH]c2ccc(-c3nc4ccc5[nH]c(C#N)cc5c4c4c3CCCC4)cc12 ; Cc1cc2c(ccc3nc(-c4ccc5[nH]nc(C(F)(F)F)c5c4)c4c(c32)CCCC4)[nH]1 ; N#Cc1cc2c(ccc3nc(-c4ccc5[nH]nc(C(F)(F)F)c5c4)c4c(c32)CCCC4)[nH]1 ; Cc1n[nH]c2ccc(-c3nc4ccc5[nH]c(C(=O)O)cc5c4c4c3CCCC4)cc12]

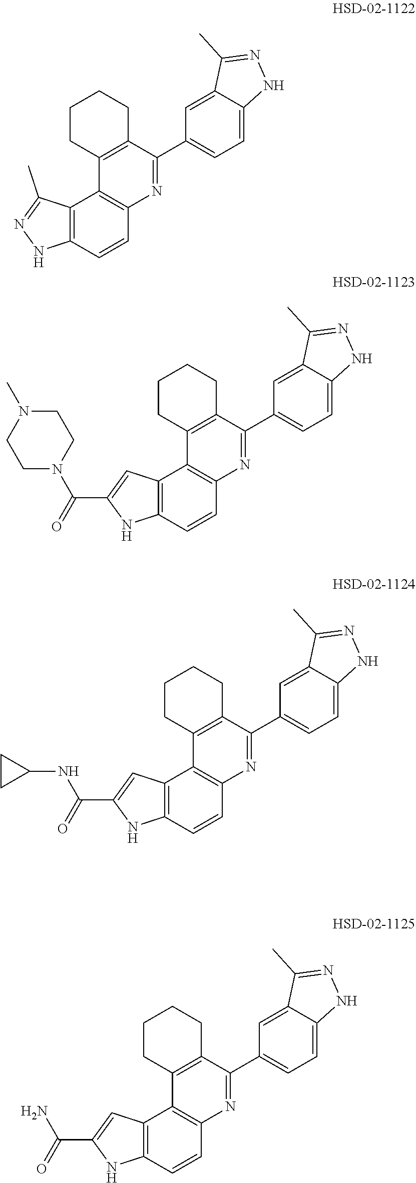 [SMILES: Cc1n[nH]c2ccc(-c3nc4ccc5[nH]nc(C)c5c4c4c3CCCC4)cc12 ; Cc1n[nH]c2ccc(-c3nc4ccc5[nH]c(C(=O)N6CCN(C)CC6)cc5c4c4c3CCCC4)cc12 ; Cc1n[nH]c2ccc(-c3nc4ccc5[nH]c(C(=O)NC6CC6)cc5c4c4c3CCCC4)cc12 ; Cc1n[nH]c2ccc(-c3nc4ccc5[nH]c(C(N)=O)cc5c4c4c3CCCC4)cc12]

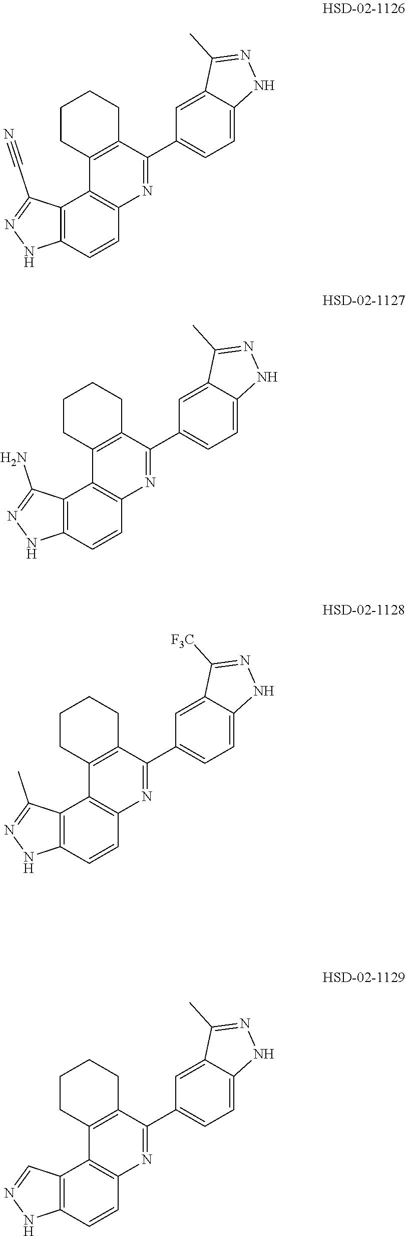 [SMILES: Cc1n[nH]c2ccc(-c3nc4ccc5[nH]nc(C#N)c5c4c4c3CCCC4)cc12 ; Cc1n[nH]c2ccc(-c3nc4ccc5[nH]nc(N)c5c4c4c3CCCC4)cc12 ; Cc1n[nH]c2ccc3nc(-c4ccc5[nH]nc(C(F)(F)F)c5c4)c4c(c3c12)CCCC4 ; Cc1n[nH]c2ccc(-c3nc4ccc5[nH]ncc5c4c4c3CCCC4)cc12]

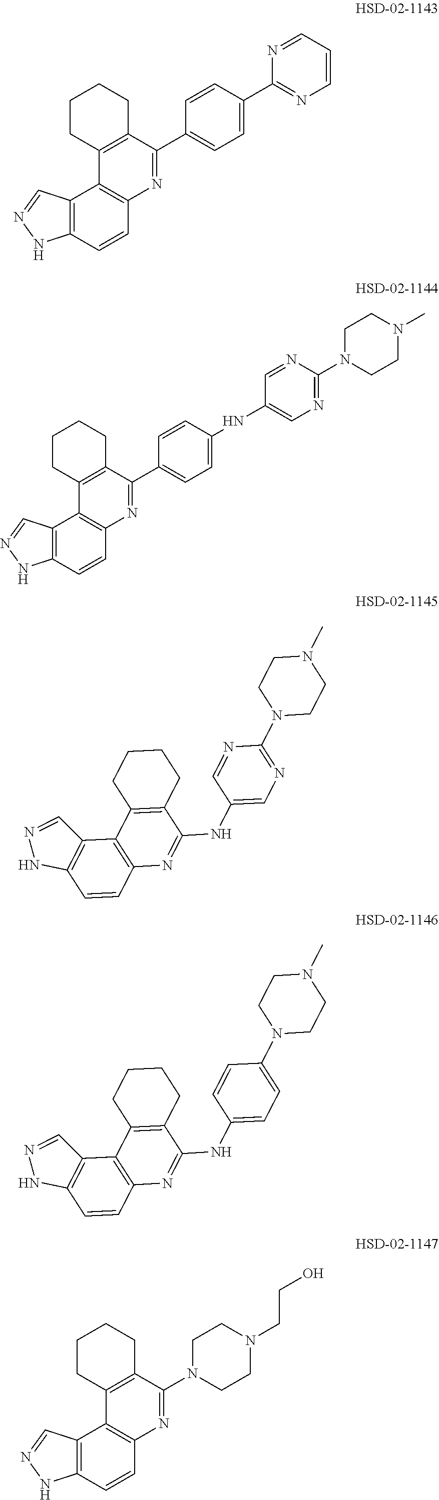 [SMILES: c1cnc(-c2ccc(-c3nc4ccc5[nH]ncc5c4c4c3CCCC4)cc2)nc1 ; CN1CCN(c2ncc(Nc3ccc(-c4nc5ccc6[nH]ncc6c5c5c4CCCC5)cc3)cn2)CC1 ; CN1CCN(c2ncc(Nc3nc4ccc5[nH]ncc5c4c4c3CCCC4)cn2)CC1 ; CN1CCN(c2ccc(Nc3nc4ccc5[nH]ncc5c4c4c3CCCC4)cc2)CC1 ; OCCN1CCN(c2nc3ccc4[nH]ncc4c3c3c2CCCC3)CC1]

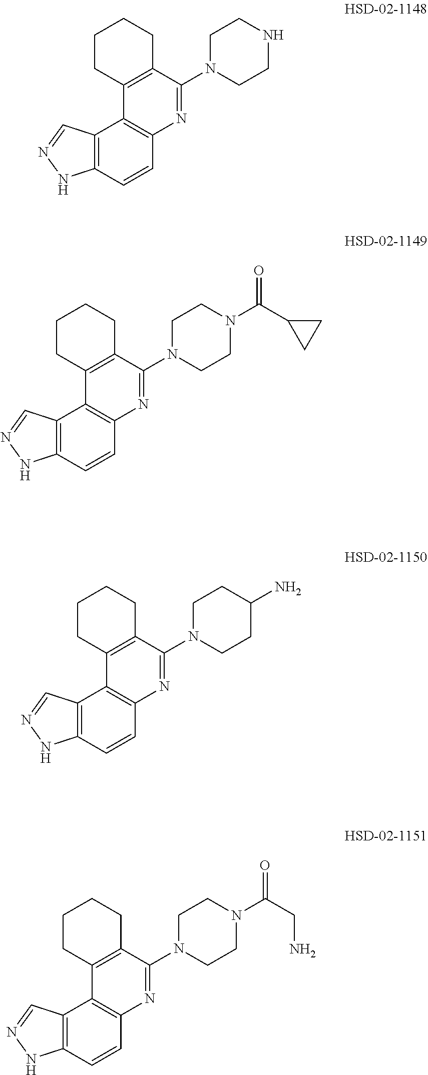 [SMILES: c1cc2[nH]ncc2c2c3c(c(N4CCNCC4)nc12)CCCC3 ; O=C(C1CC1)N1CCN(c2nc3ccc4[nH]ncc4c3c3c2CCCC3)CC1 ; NC1CCN(c2nc3ccc4[nH]ncc4c3c3c2CCCC3)CC1 ; NCC(=O)N1CCN(c2nc3ccc4[nH]ncc4c3c3c2CCCC3)CC1]

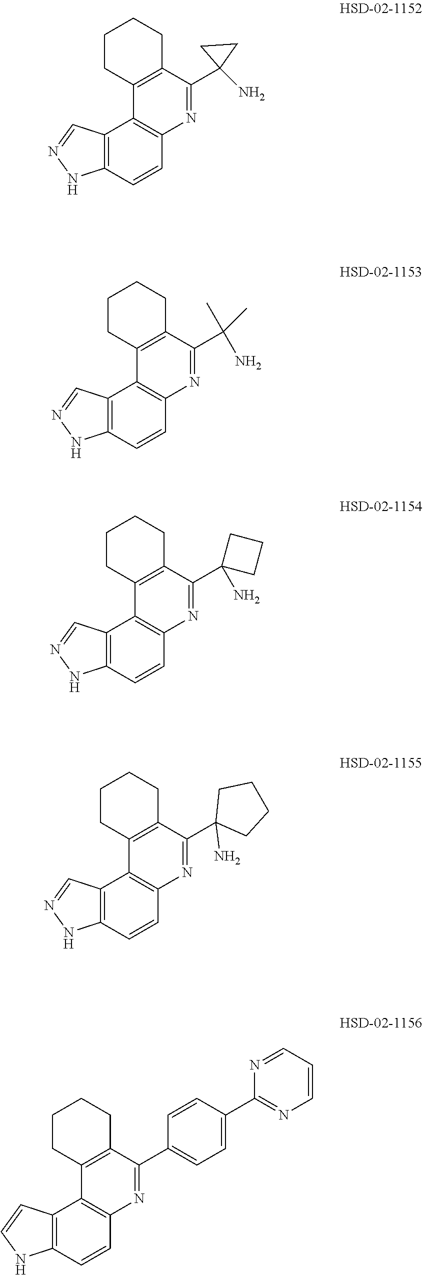 [SMILES: NC1(c2nc3ccc4[nH]ncc4c3c3c2CCCC3)CC1 ; CC(C)(N)c1nc2ccc3[nH]ncc3c2c2c1CCCC2 ; NC1(c2nc3ccc4[nH]ncc4c3c3c2CCCC3)CCC1 ; NC1(c2nc3ccc4[nH]ncc4c3c3c2CCCC3)CCCC1 ; c1cnc(-c2ccc(-c3nc4ccc5[nH]ccc5c4c4c3CCCC4)cc2)nc1]

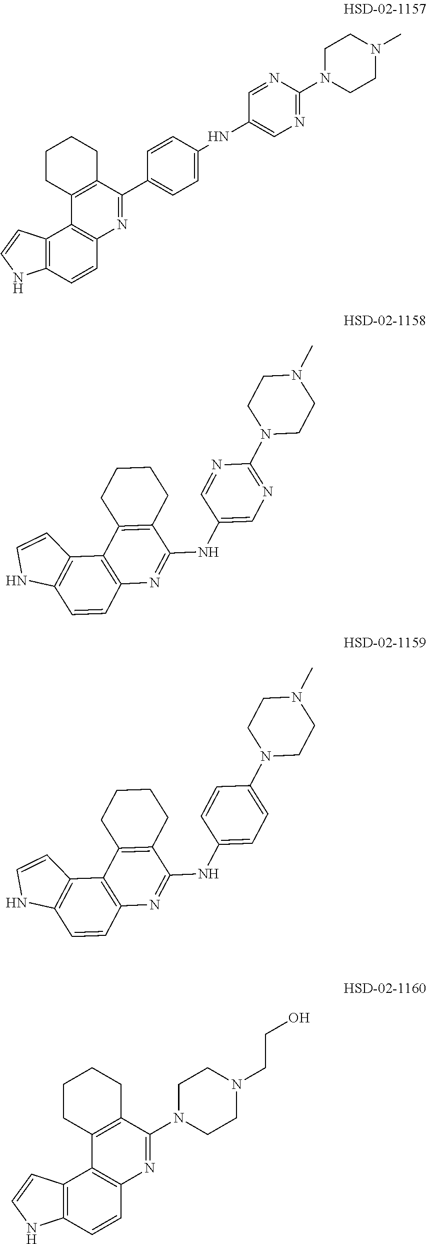 [SMILES: CN1CCN(c2ncc(Nc3ccc(-c4nc5ccc6[nH]ccc6c5c5c4CCCC5)cc3)cn2)CC1 ; CN1CCN(c2ncc(Nc3nc4ccc5[nH]ccc5c4c4c3CCCC4)cn2)CC1 ; CN1CCN(c2ccc(Nc3nc4ccc5[nH]ccc5c4c4c3CCCC4)cc2)CC1 ; OCCN1CCN(c2nc3ccc4[nH]ccc4c3c3c2CCCC3)CC1]

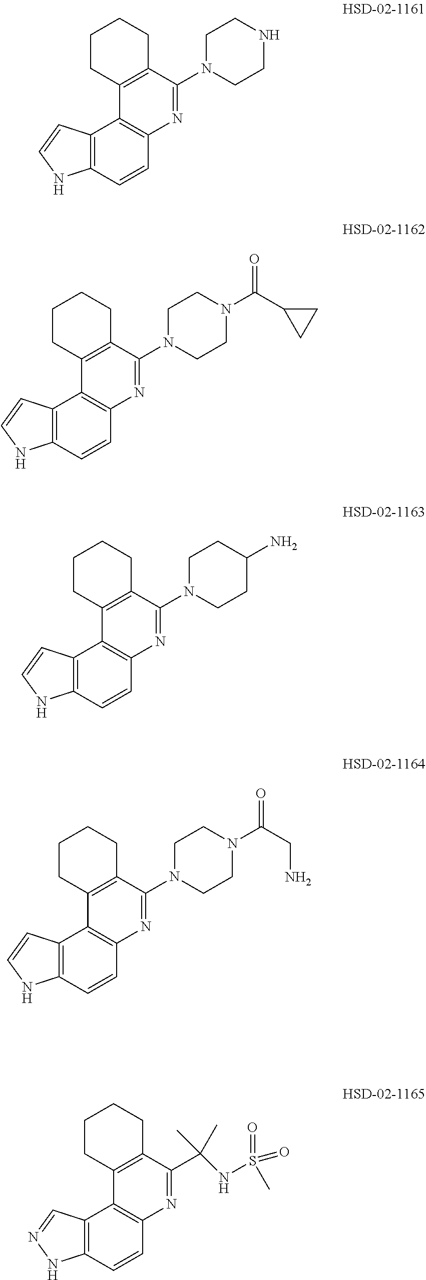 [SMILES: c1cc2c(ccc3nc(N4CCNCC4)c4c(c32)CCCC4)[nH]1 ; O=C(C1CC1)N1CCN(c2nc3ccc4[nH]ccc4c3c3c2CCCC3)CC1 ; NC1CCN(c2nc3ccc4[nH]ccc4c3c3c2CCCC3)CC1 ; NCC(=O)N1CCN(c2nc3ccc4[nH]ccc4c3c3c2CCCC3)CC1 ; CC(C)(NS(C)(=O)=O)c1nc2ccc3[nH]ncc3c2c2c1CCCC2]

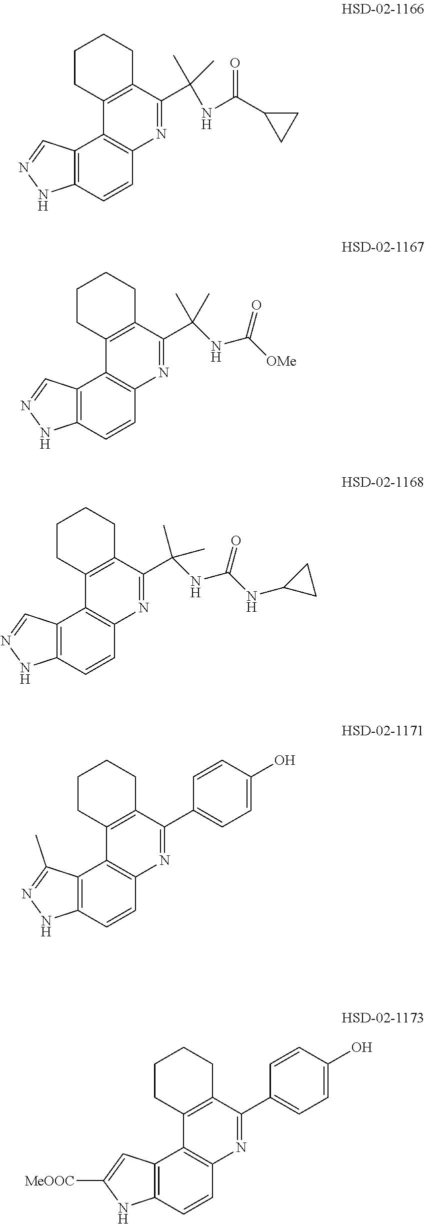 [SMILES: CC(C)(NC(=O)C1CC1)c1nc2ccc3[nH]ncc3c2c2c1CCCC2 ; COC(=O)NC(C)(C)c1nc2ccc3[nH]ncc3c2c2c1CCCC2 ; CC(C)(NC(=O)NC1CC1)c1nc2ccc3[nH]ncc3c2c2c1CCCC2 ; Cc1n[nH]c2ccc3nc(-c4ccc(O)cc4)c4c(c3c12)CCCC4 ; COC(=O)c1cc2c(ccc3nc(-c4ccc(O)cc4)c4c(c32)CCCC4)[nH]1]

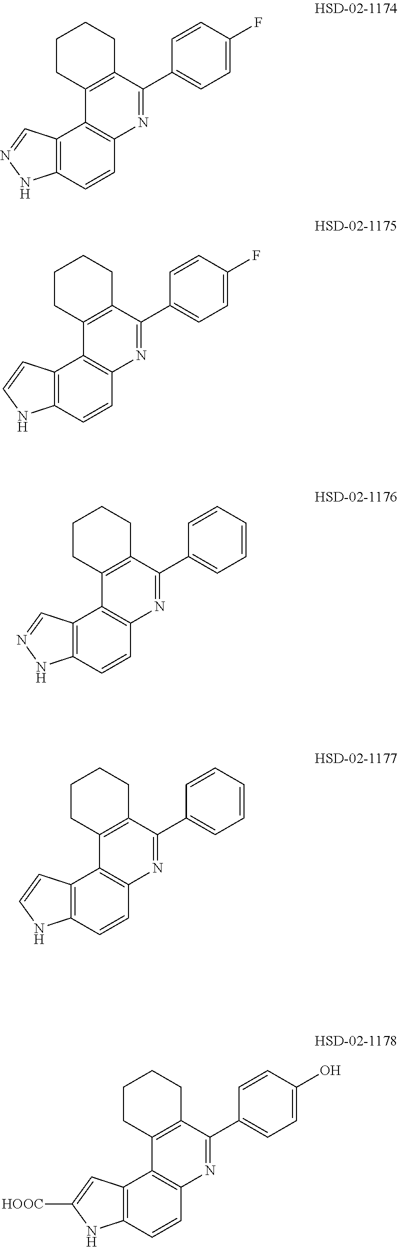 [SMILES: Fc1ccc(-c2nc3ccc4[nH]ncc4c3c3c2CCCC3)cc1 ; Fc1ccc(-c2nc3ccc4[nH]ccc4c3c3c2CCCC3)cc1 ; c1ccc(-c2nc3ccc4[nH]ncc4c3c3c2CCCC3)cc1 ; c1ccc(-c2nc3ccc4[nH]ccc4c3c3c2CCCC3)cc1 ; O=C(O)c1cc2c(ccc3nc(-c4ccc(O)cc4)c4c(c32)CCCC4)[nH]1]

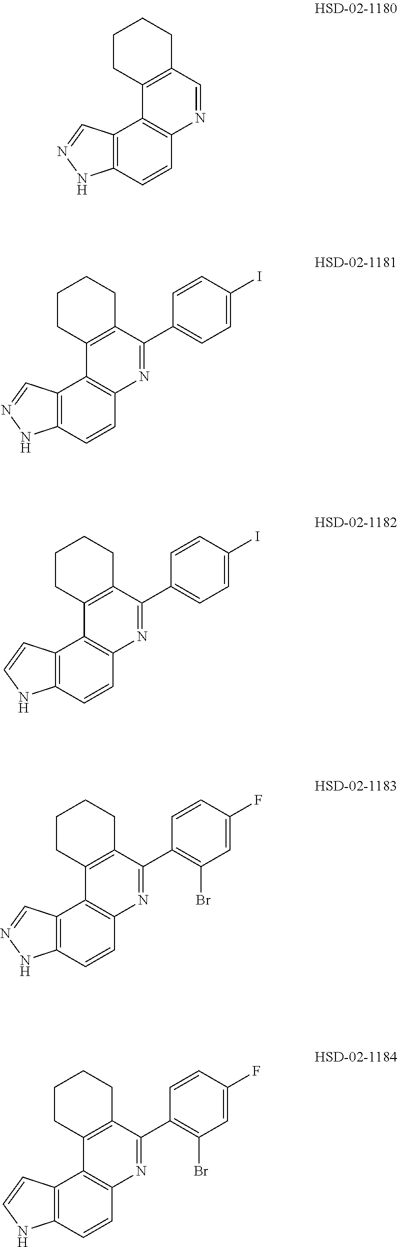 [SMILES: c1nc2ccc3[nH]ncc3c2c2c1CCCC2 ; Ic1ccc(-c2nc3ccc4[nH]ncc4c3c3c2CCCC3)cc1 ; Ic1ccc(-c2nc3ccc4[nH]ccc4c3c3c2CCCC3)cc1 ; Fc1ccc(-c2nc3ccc4[nH]ncc4c3c3c2CCCC3)c(Br)c1 ; Fc1ccc(-c2nc3ccc4[nH]ccc4c3c3c2CCCC3)c(Br)c1]

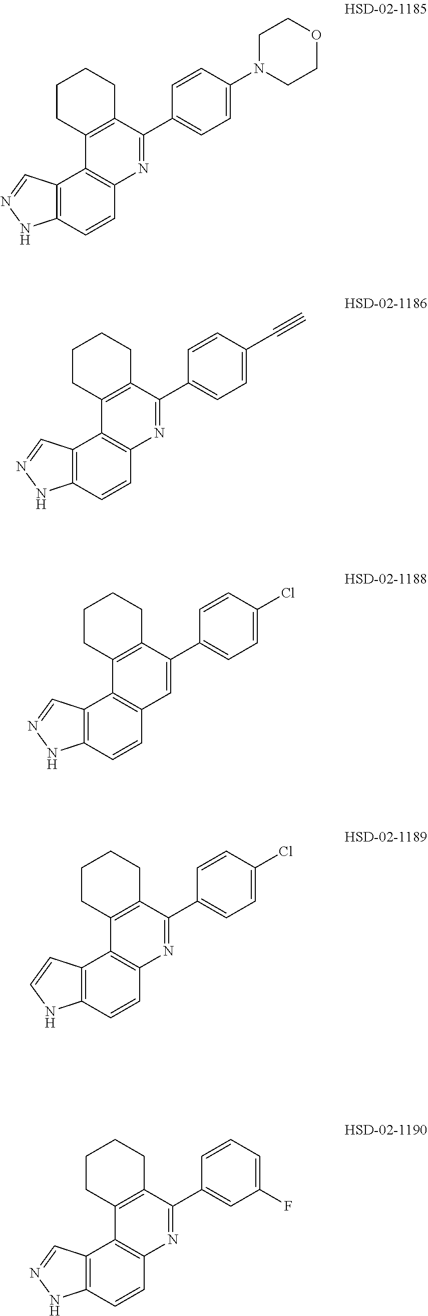 [SMILES: c1cc(N2CCOCC2)ccc1-c1nc2ccc3[nH]ncc3c2c2c1CCCC2 ; C#Cc1ccc(-c2nc3ccc4[nH]ncc4c3c3c2CCCC3)cc1 ; Clc1ccc(-c2cc3ccc4[nH]ncc4c3c3c2CCCC3)cc1 ; Clc1ccc(-c2nc3ccc4[nH]ccc4c3c3c2CCCC3)cc1 ; Fc1cccc(-c2nc3ccc4[nH]ncc4c3c3c2CCCC3)c1]

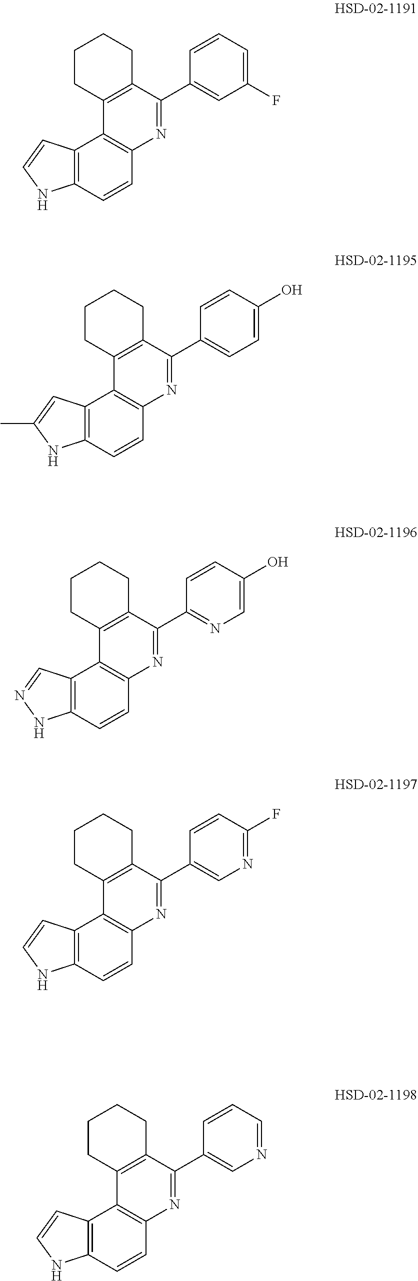 [SMILES: Fc1cccc(-c2nc3ccc4[nH]ccc4c3c3c2CCCC3)c1 ; Cc1cc2c(ccc3nc(-c4ccc(O)cc4)c4c(c32)CCCC4)[nH]1 ; Oc1ccc(-c2nc3ccc4[nH]ncc4c3c3c2CCCC3)nc1 ; Fc1ccc(-c2nc3ccc4[nH]ccc4c3c3c2CCCC3)cn1 ; c1cncc(-c2nc3ccc4[nH]ccc4c3c3c2CCCC3)c1]

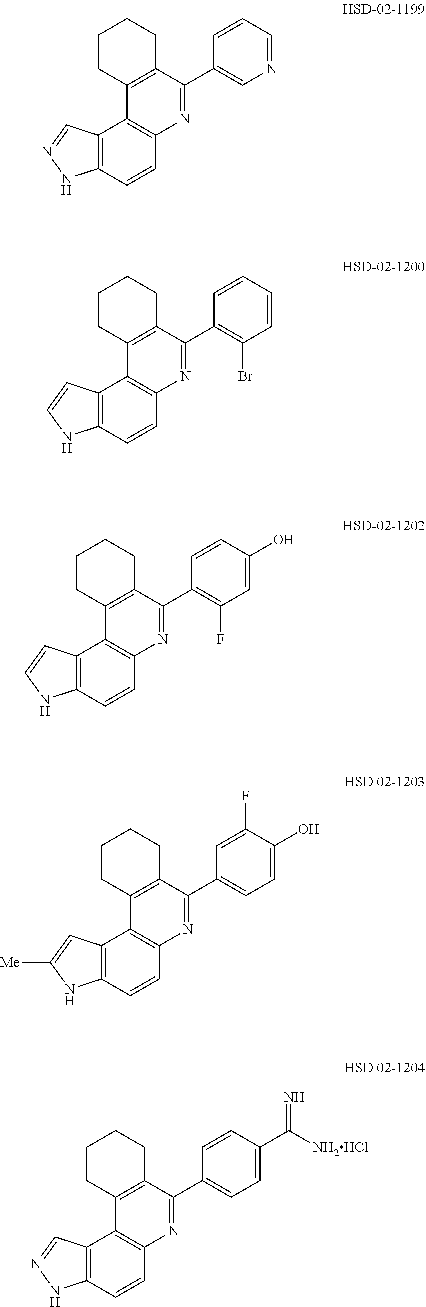

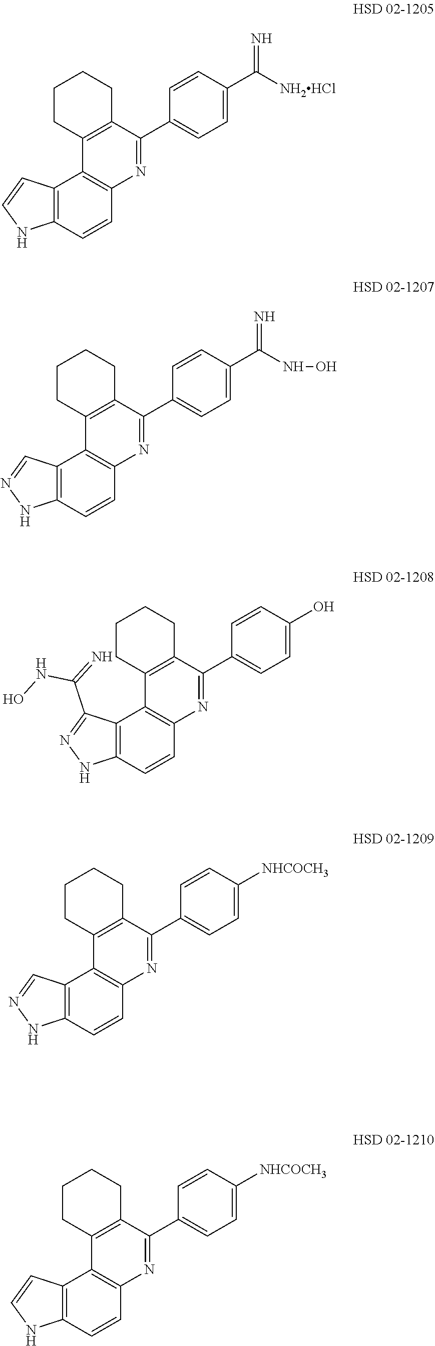

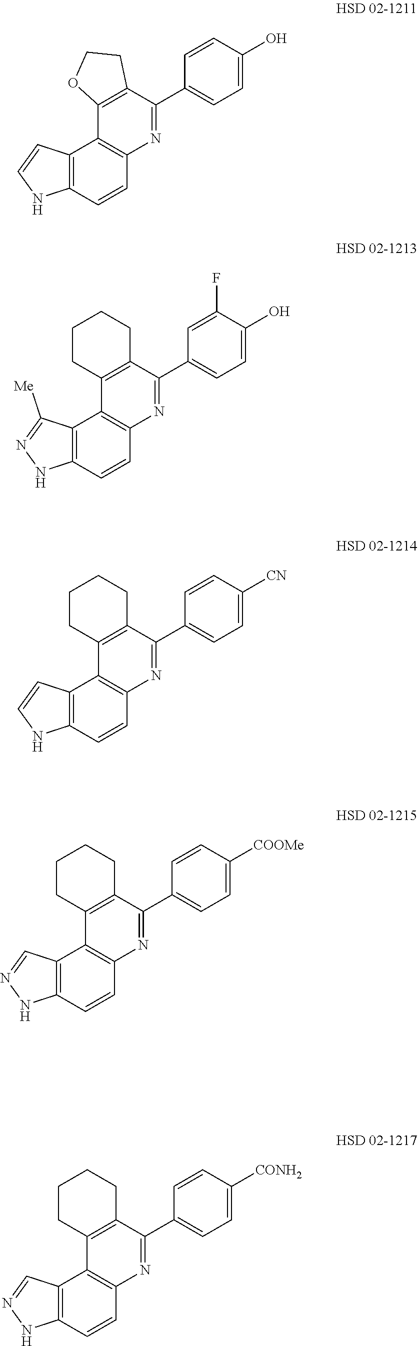 [SMILES: Oc1ccc(-c2nc3ccc4[nH]ccc4c3c3c2CCO3)cc1 ; Cc1n[nH]c2ccc3nc(-c4ccc(O)c(F)c4)c4c(c3c12)CCCC4 ; N#Cc1ccc(-c2nc3ccc4[nH]ccc4c3c3c2CCCC3)cc1 ; COC(=O)c1ccc(-c2nc3ccc4[nH]ncc4c3c3c2CCCC3)cc1 ; NC(=O)c1ccc(-c2nc3ccc4[nH]ncc4c3c3c2CCCC3)cc1]

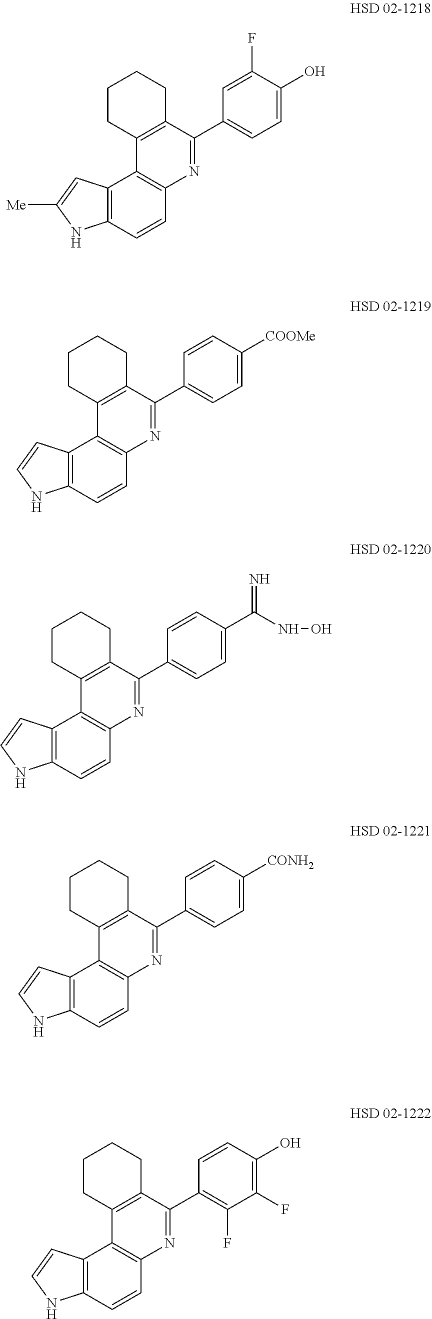 [SMILES: Cc1cc2c(ccc3nc(-c4ccc(O)c(F)c4)c4c(c32)CCCC4)[nH]1 ; COC(=O)c1ccc(-c2nc3ccc4[nH]ccc4c3c3c2CCCC3)cc1 ; N=C(NO)c1ccc(-c2nc3ccc4[nH]ccc4c3c3c2CCCC3)cc1 ; NC(=O)c1ccc(-c2nc3ccc4[nH]ccc4c3c3c2CCCC3)cc1 ; Oc1ccc(-c2nc3ccc4[nH]ccc4c3c3c2CCCC3)c(F)c1F]

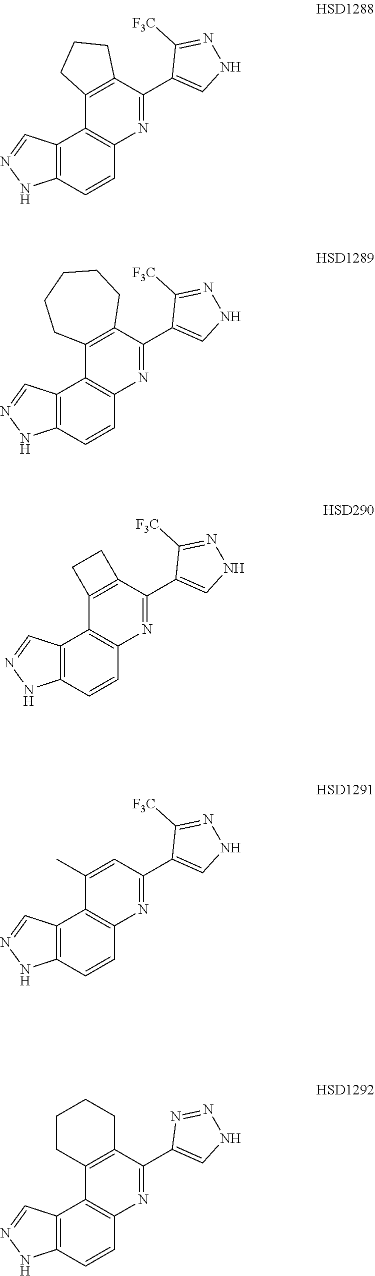 [SMILES: FC(F)(F)c1n[nH]cc1-c1nc2ccc3[nH]ncc3c2c2c1CCC2 ; FC(F)(F)c1n[nH]cc1-c1nc2ccc3[nH]ncc3c2c2c1CCCCC2 ; FC(F)(F)c1n[nH]cc1-c1nc2ccc3[nH]ncc3c2c2c1CC2 ; Cc1cc(-c2c[nH]nc2C(F)(F)F)nc2ccc3[nH]ncc3c12 ; c1[nH]nnc1-c1nc2ccc3[nH]ncc3c2c2c1CCCC2]

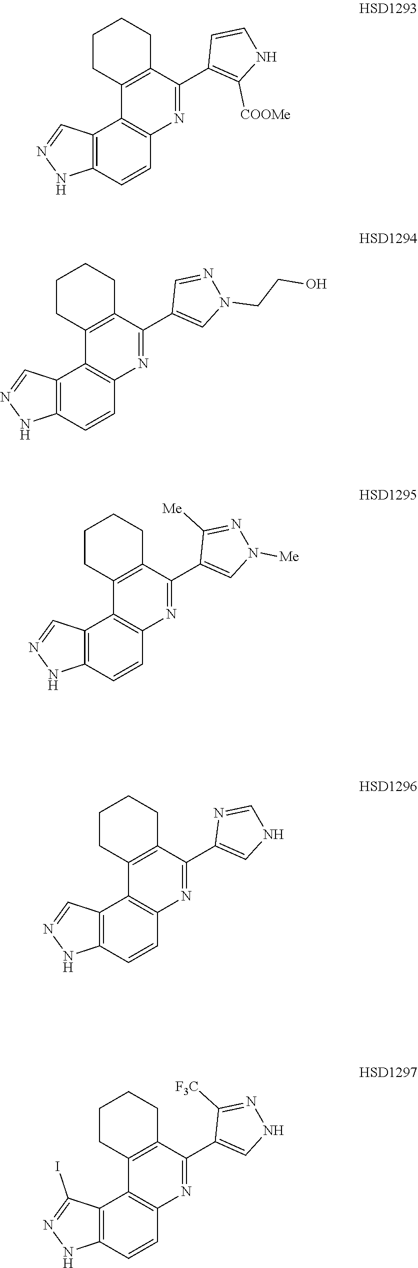 [SMILES: COC(=O)c1[nH]ccc1-c1nc2ccc3[nH]ncc3c2c2c1CCCC2 ; OCCn1cc(-c2nc3ccc4[nH]ncc4c3c3c2CCCC3)cn1 ; Cc1nn(C)cc1-c1nc2ccc3[nH]ncc3c2c2c1CCCC2 ; c1nc(-c2nc3ccc4[nH]ncc4c3c3c2CCCC3)c[nH]1 ; FC(F)(F)c1n[nH]cc1-c1nc2ccc3[nH]nc(I)c3c2c2c1CCCC2]

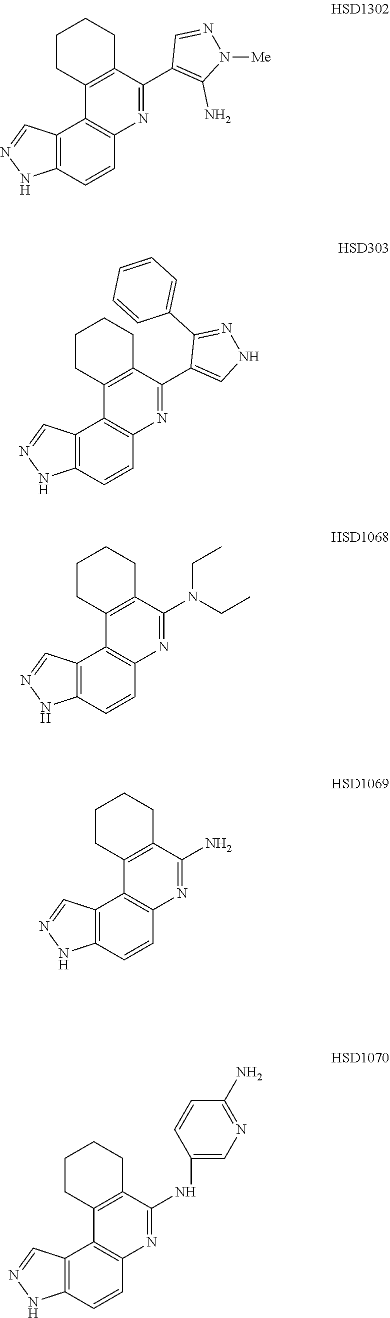 [SMILES: Cn1ncc(-c2nc3ccc4[nH]ncc4c3c3c2CCCC3)c1N ; c1ccc(-c2n[nH]cc2-c2nc3ccc4[nH]ncc4c3c3c2CCCC3)cc1 ; CCN(CC)c1nc2ccc3[nH]ncc3c2c2c1CCCC2 ; Nc1nc2ccc3[nH]ncc3c2c2c1CCCC2 ; Nc1ccc(Nc2nc3ccc4[nH]ncc4c3c3c2CCCC3)cn1]

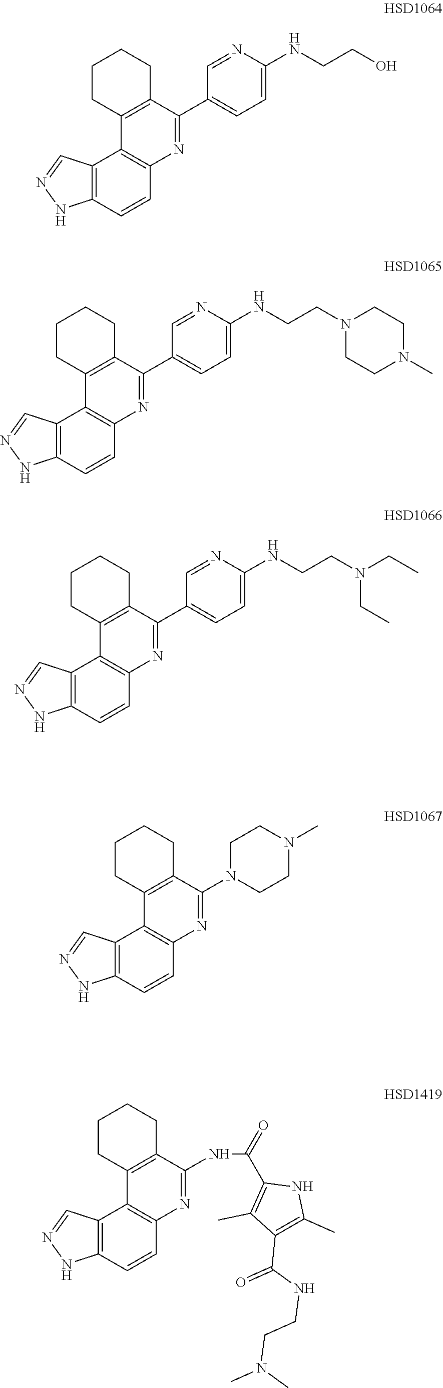 [SMILES: OCCNc1ccc(-c2nc3ccc4[nH]ncc4c3c3c2CCCC3)cn1 ; CN1CCN(CCNc2ccc(-c3nc4ccc5[nH]ncc5c4c4c3CCCC4)cn2)CC1 ; CCN(CC)CCNc1ccc(-c2nc3ccc4[nH]ncc4c3c3c2CCCC3)cn1 ; CN1CCN(c2nc3ccc4[nH]ncc4c3c3c2CCCC3)CC1 ; Cc1[nH]c(C(=O)Nc2nc3ccc4[nH]ncc4c3c3c2CCCC3)c(C)c1C(=O)NCCN(C)C]

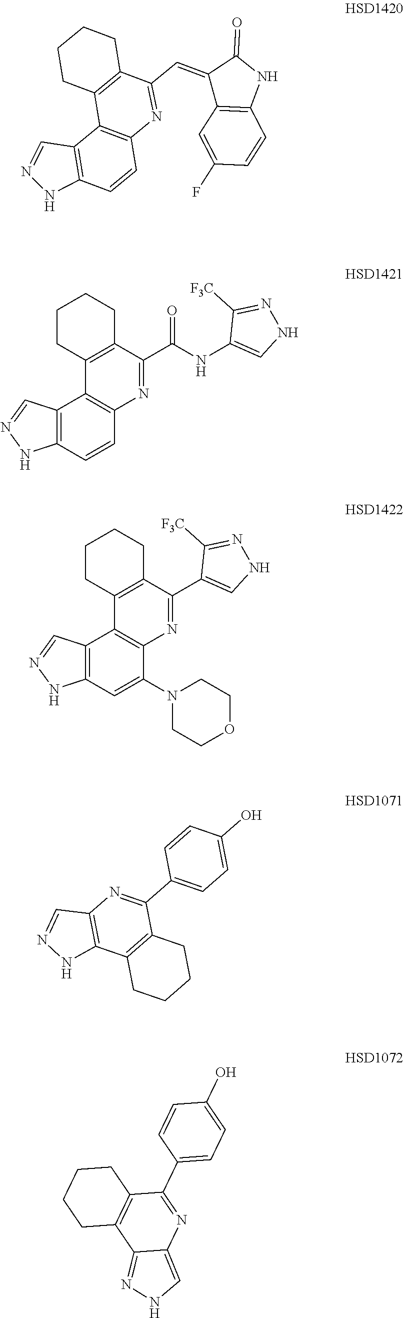 [SMILES: O=C1Nc2ccc(F)cc2/C1=C\c1nc2ccc3[nH]ncc3c2c2c1CCCC2 ; O=C(Nc1c[nH]nc1C(F)(F)F)c1nc2ccc3[nH]ncc3c2c2c1CCCC2 ; FC(F)(F)c1n[nH]cc1-c1nc2c(N3CCOCC3)cc3[nH]ncc3c2c2c1CCCC2 ; Oc1ccc(-c2nc3cn[nH]c3c3c2CCCC3)cc1 ; Oc1ccc(-c2nc3c[nH]nc3c3c2CCCC3)cc1]

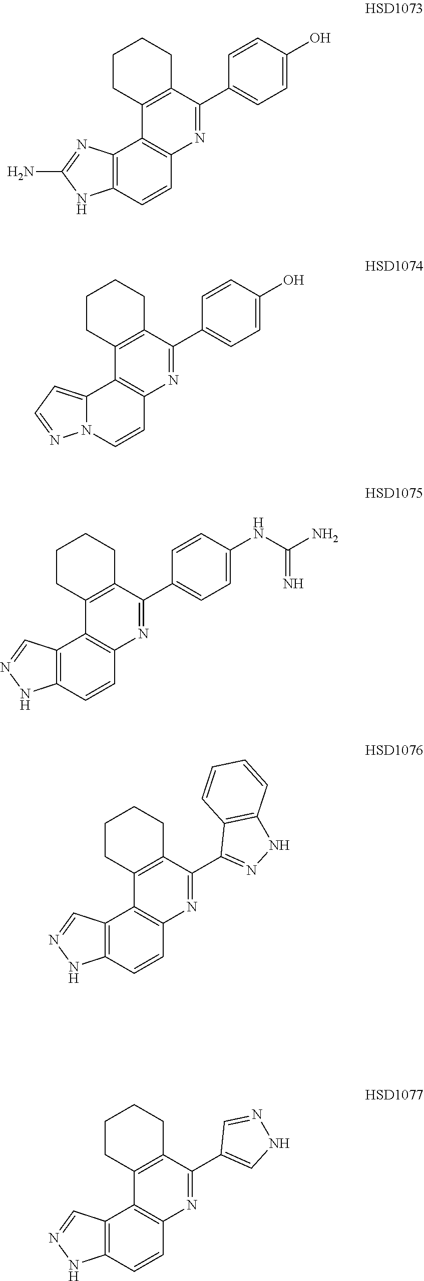 [SMILES: Nc1nc2c(ccc3nc(-c4ccc(O)cc4)c4c(c32)CCCC4)[nH]1 ; Oc1ccc(-c2nc3ccn4nccc4c3c3c2CCCC3)cc1 ; N=C(N)Nc1ccc(-c2nc3ccc4[nH]ncc4c3c3c2CCCC3)cc1 ; c1ccc2c(-c3nc4ccc5[nH]ncc5c4c4c3CCCC4)n[nH]c2c1 ; c1n[nH]cc1-c1nc2ccc3[nH]ncc3c2c2c1CCCC2]

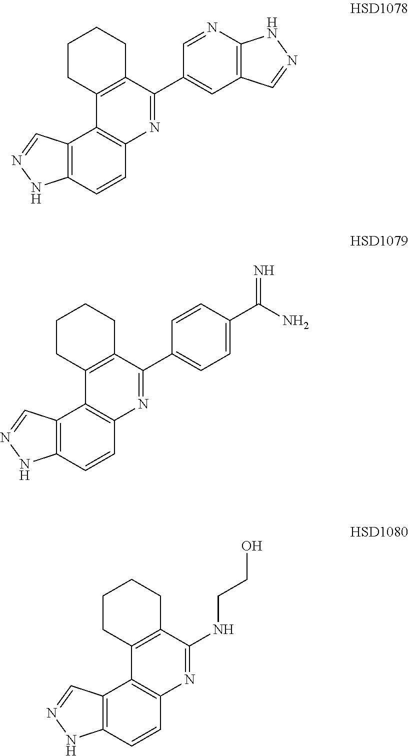 [SMILES: c1nc2[nH]ncc2cc1-c1nc2ccc3[nH]ncc3c2c2c1CCCC2 ; N=C(N)c1ccc(-c2nc3ccc4[nH]ncc4c3c3c2CCCC3)cc1 ; OCCNc1nc2ccc3[nH]ncc3c2c2c1CCCC2]

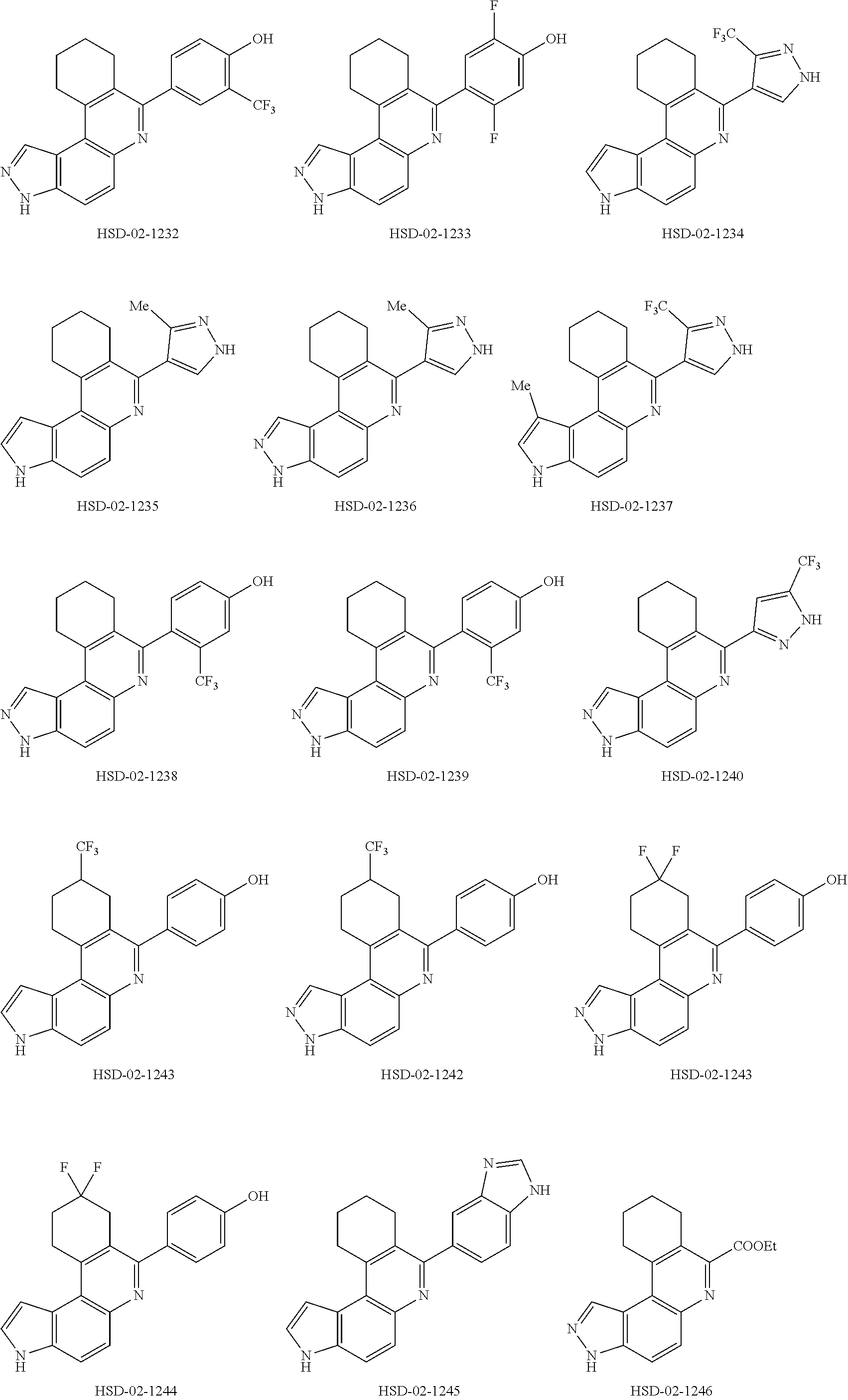

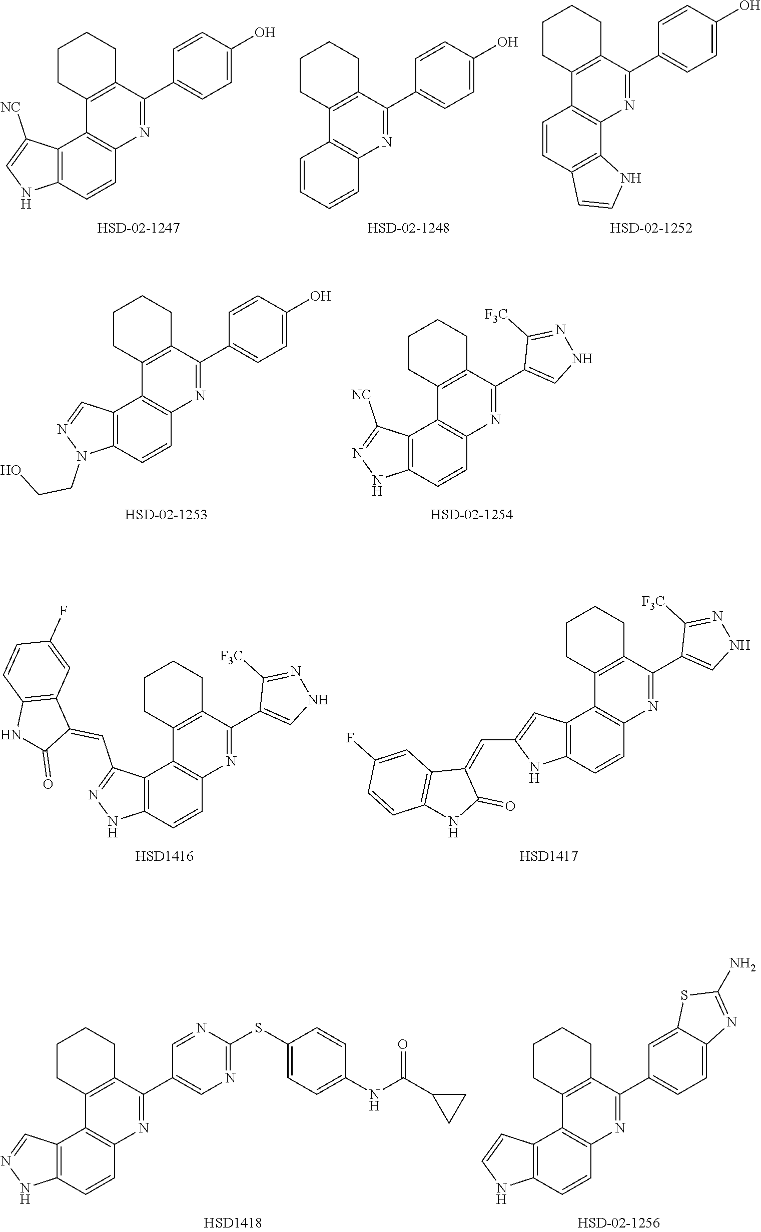

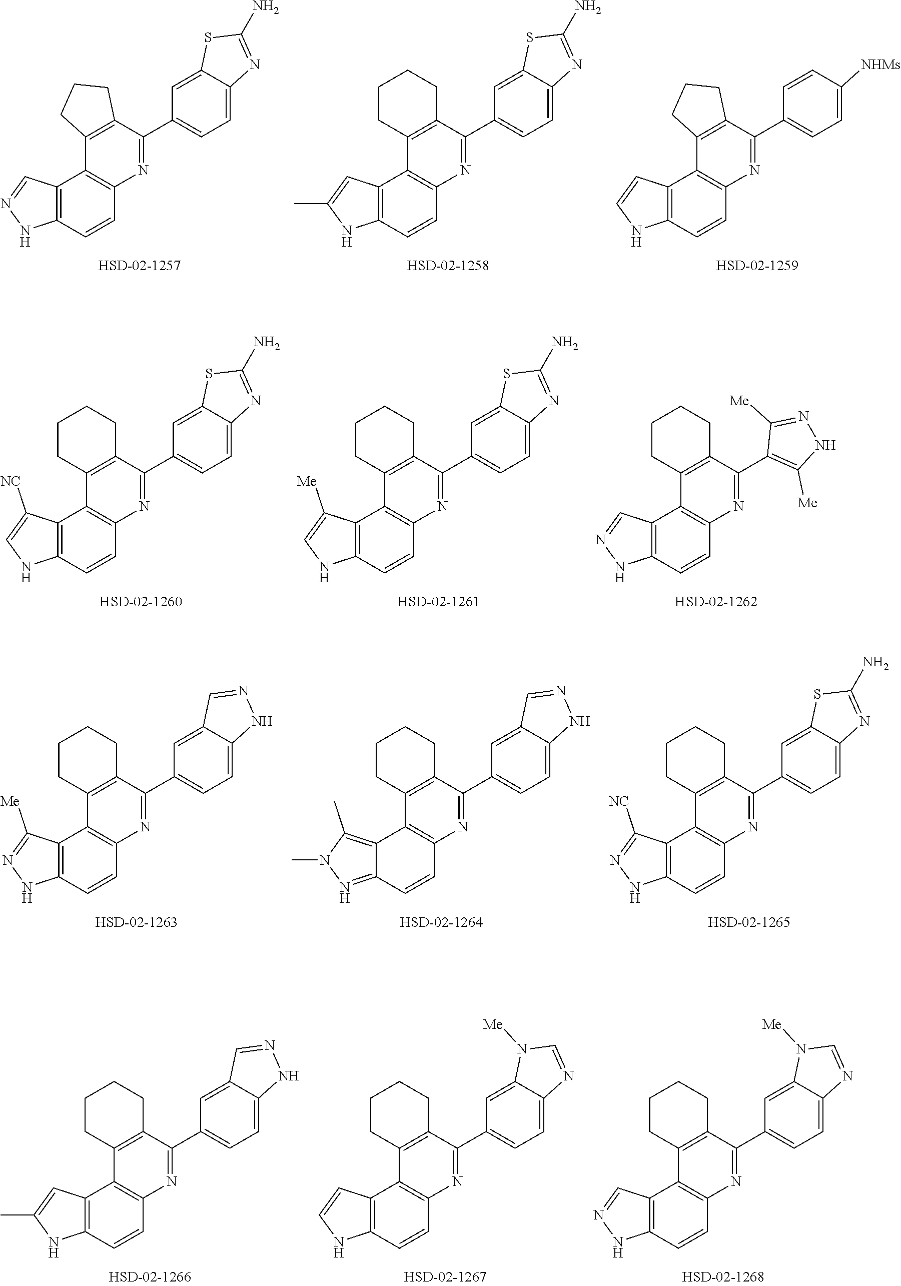

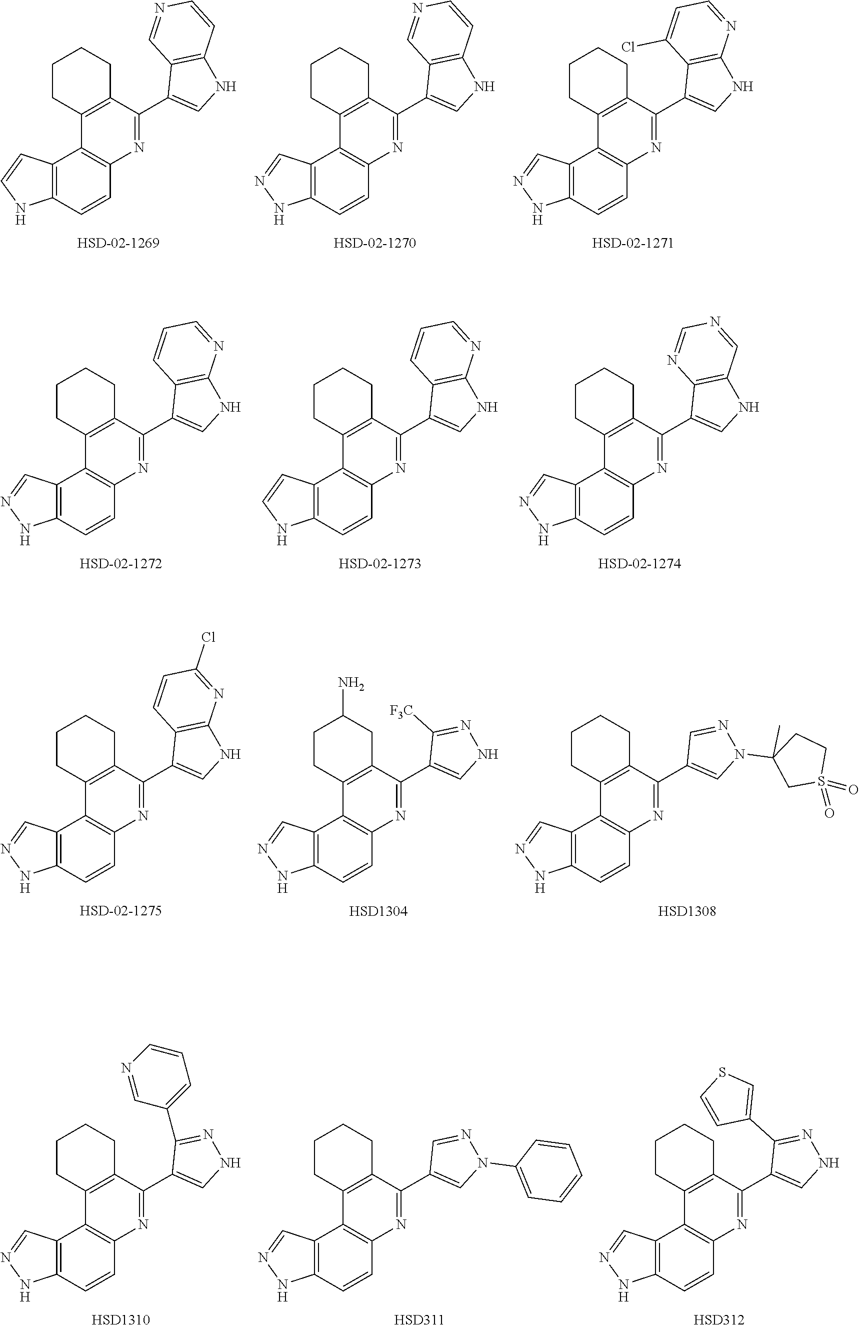

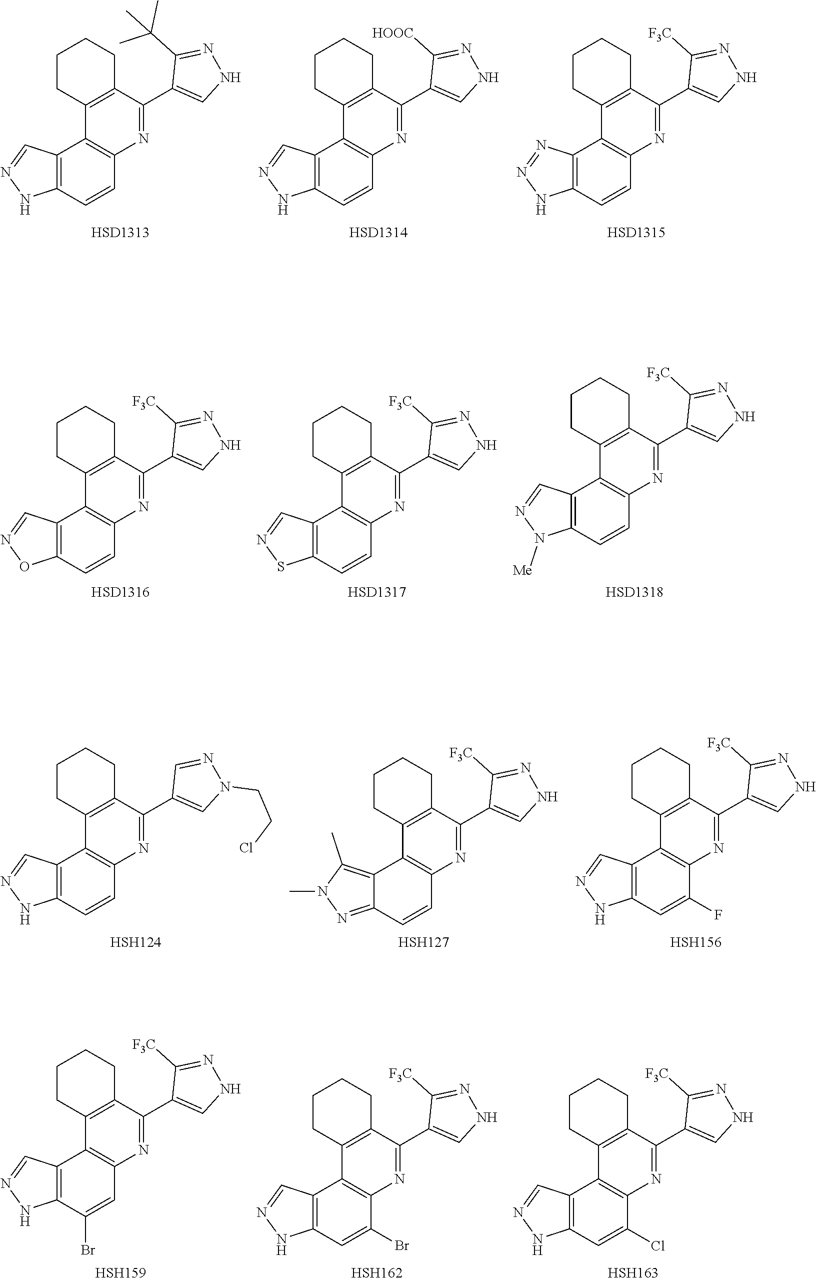

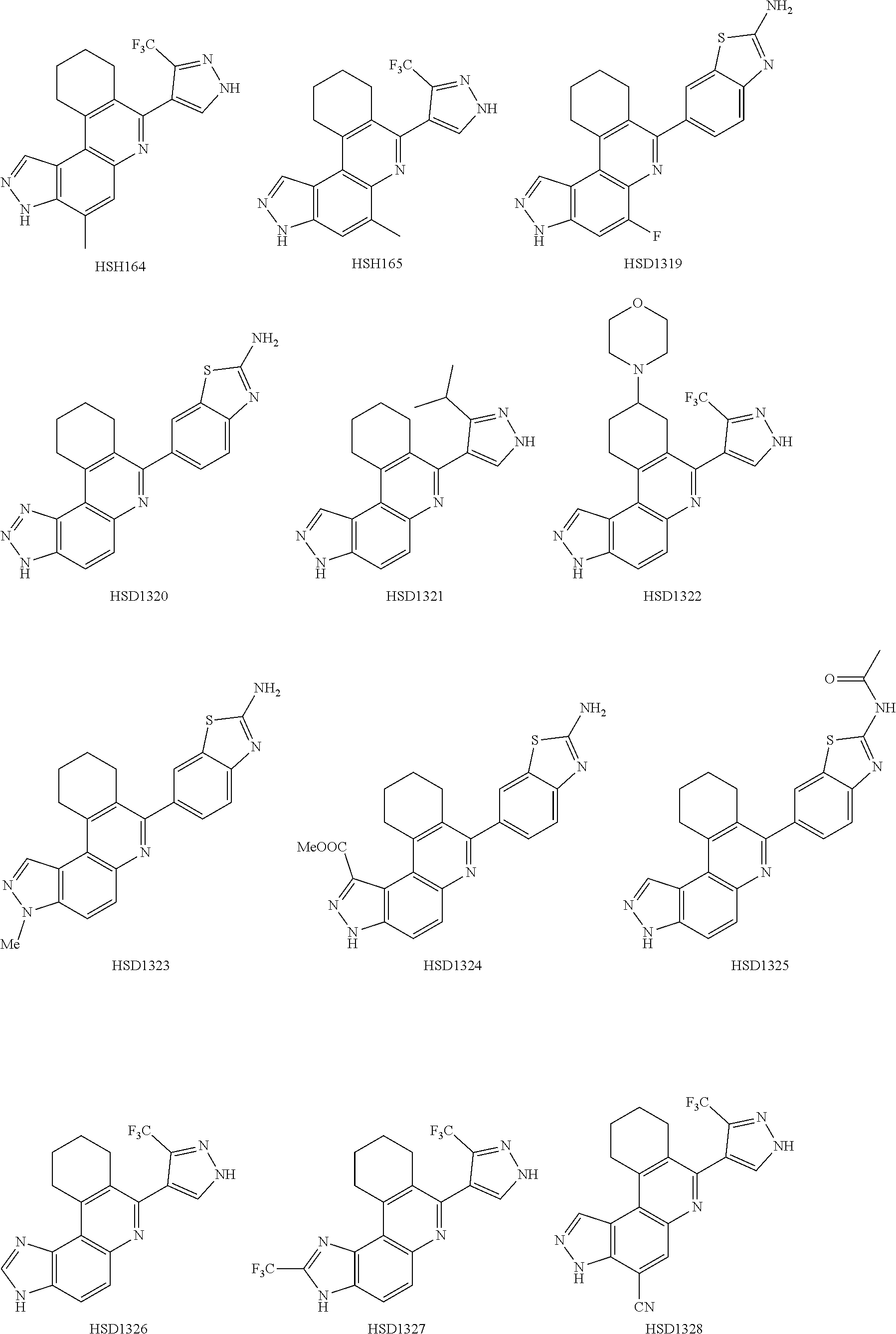

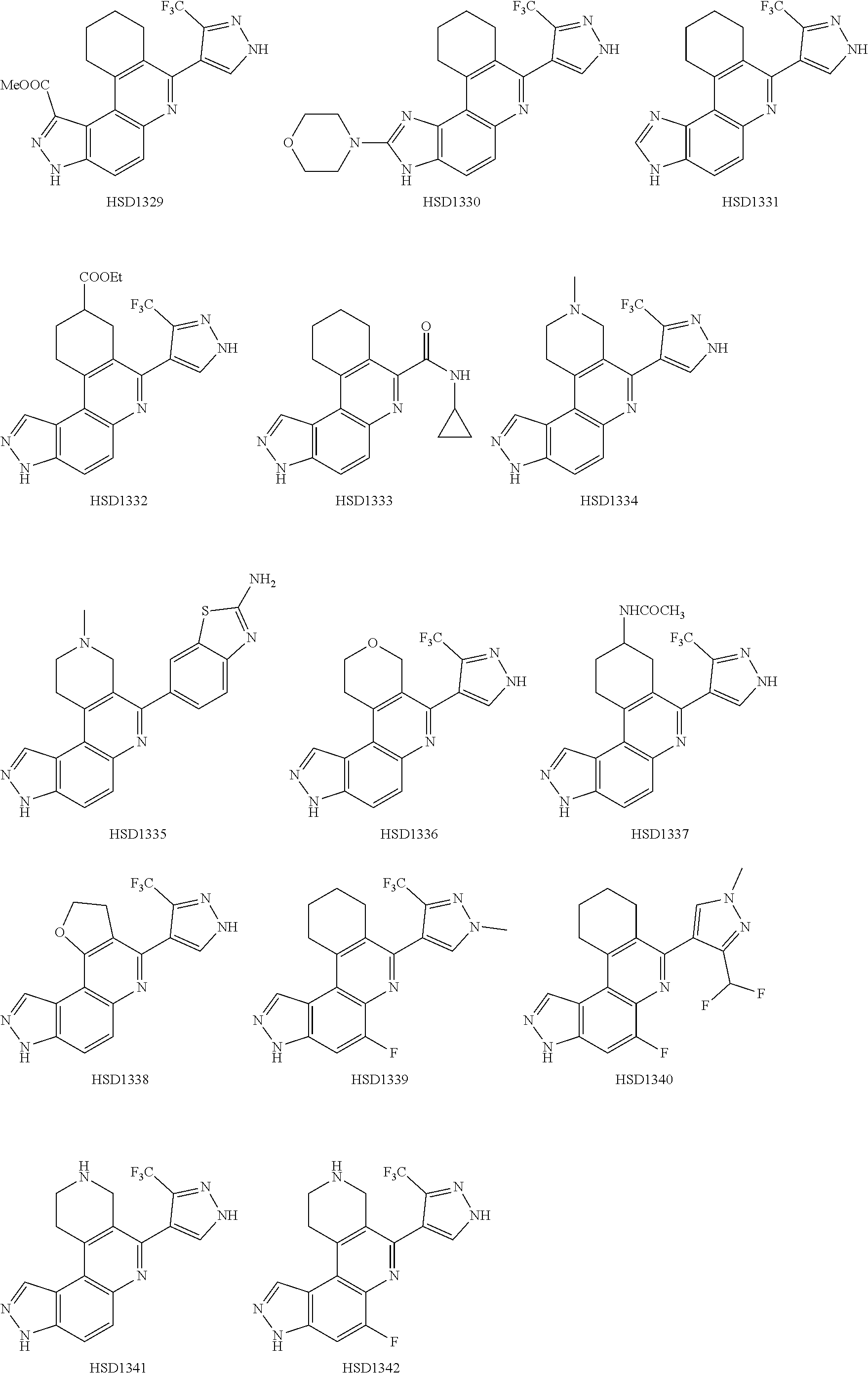

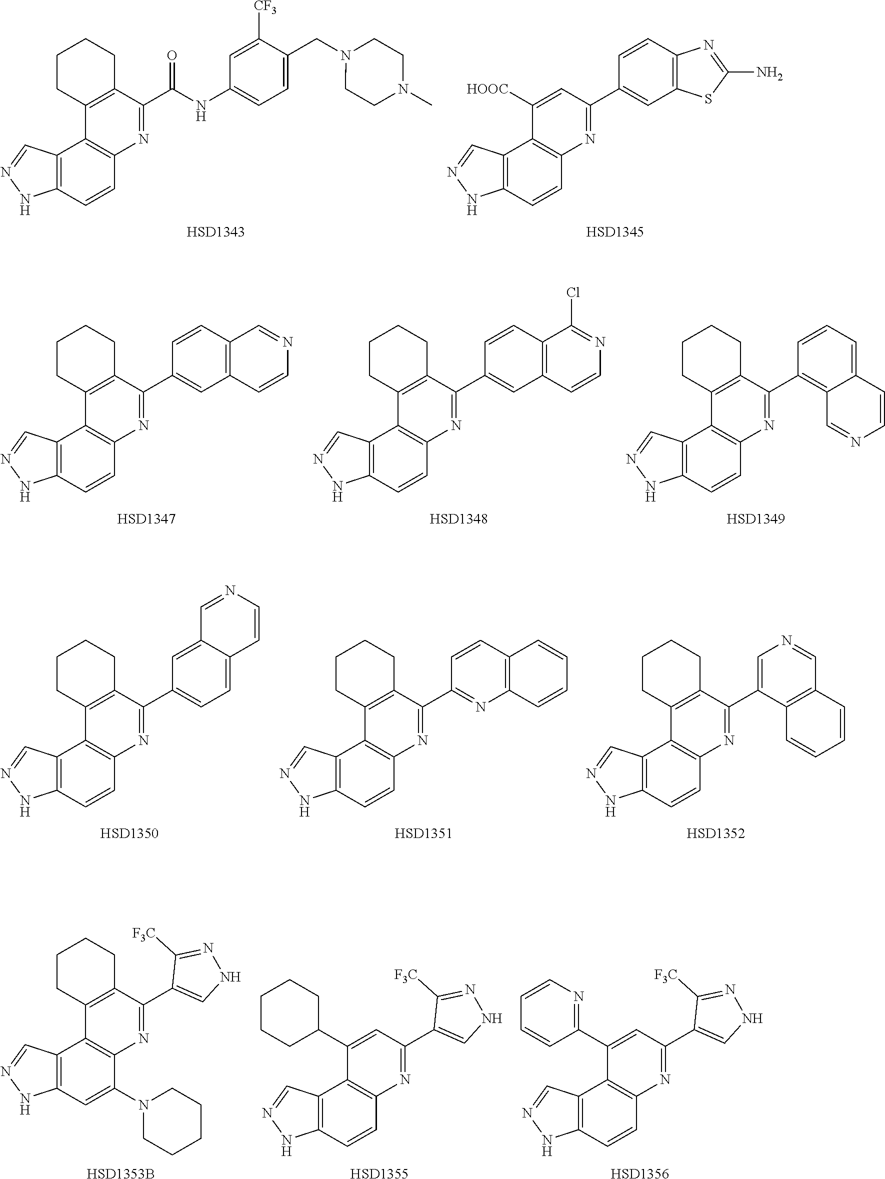

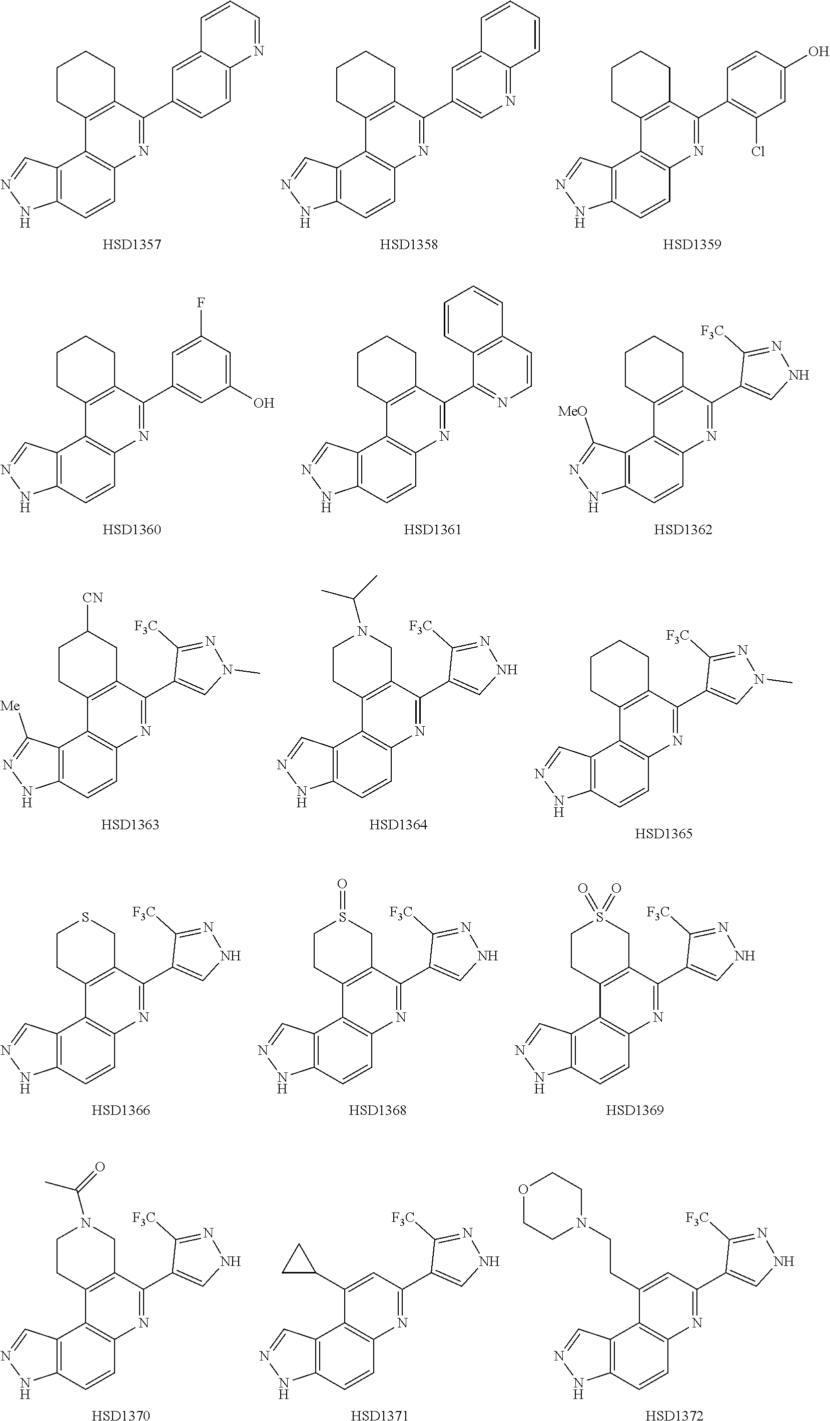

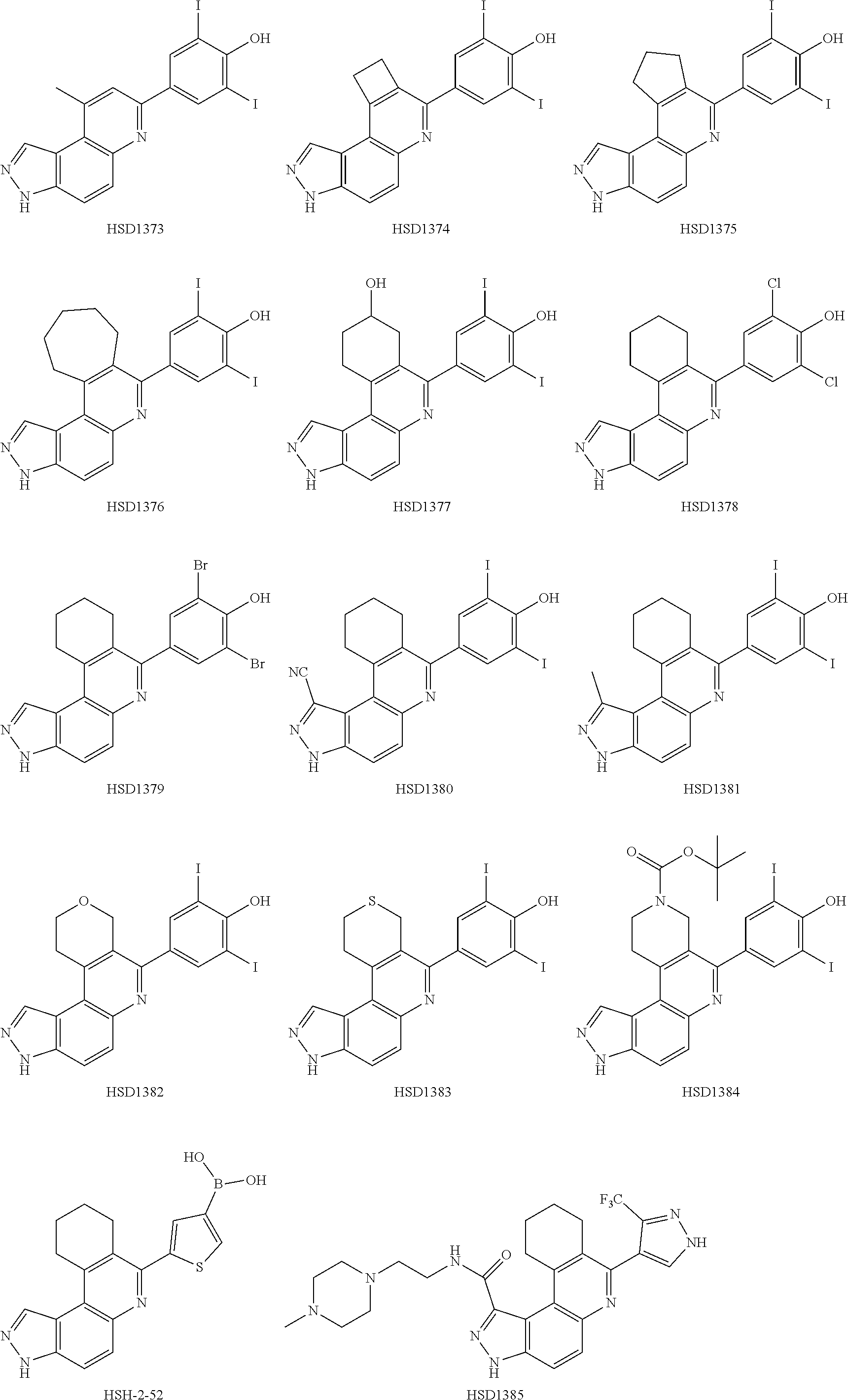

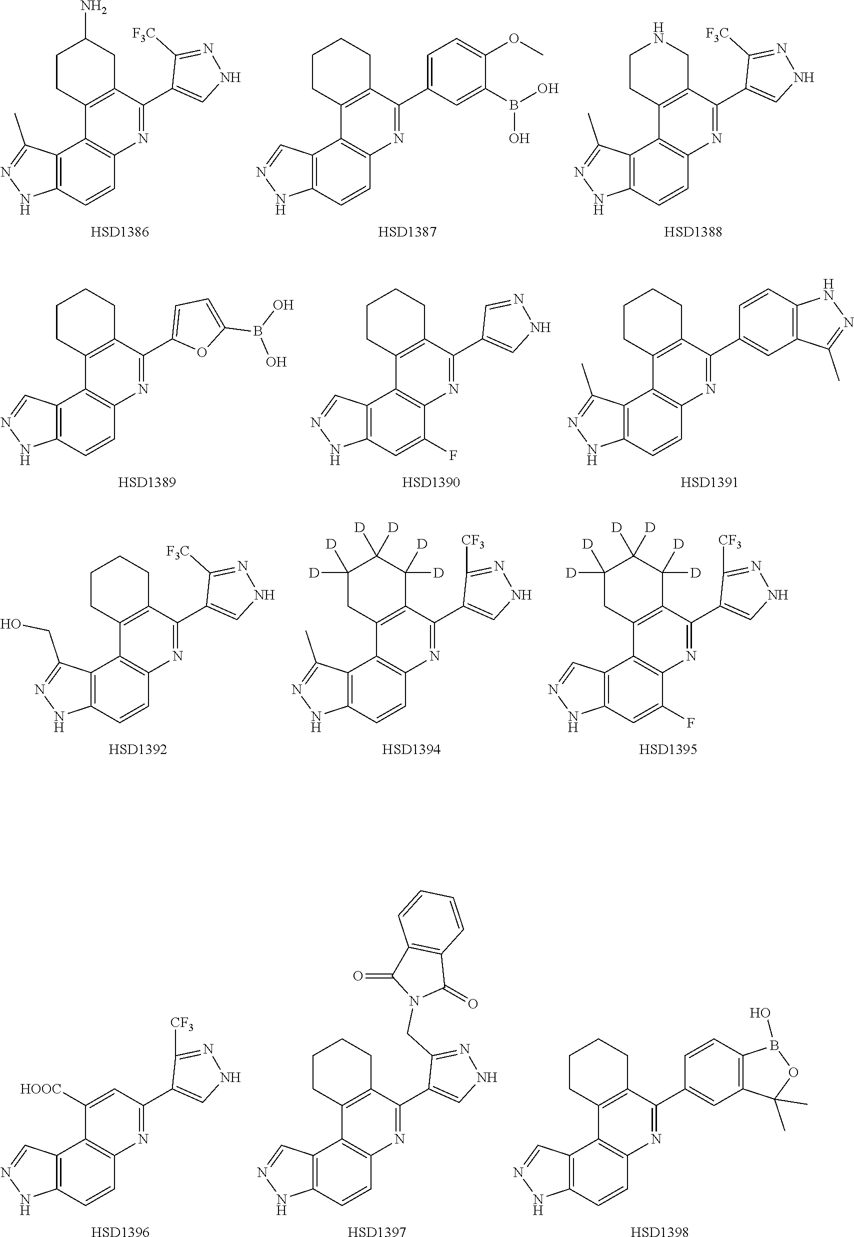

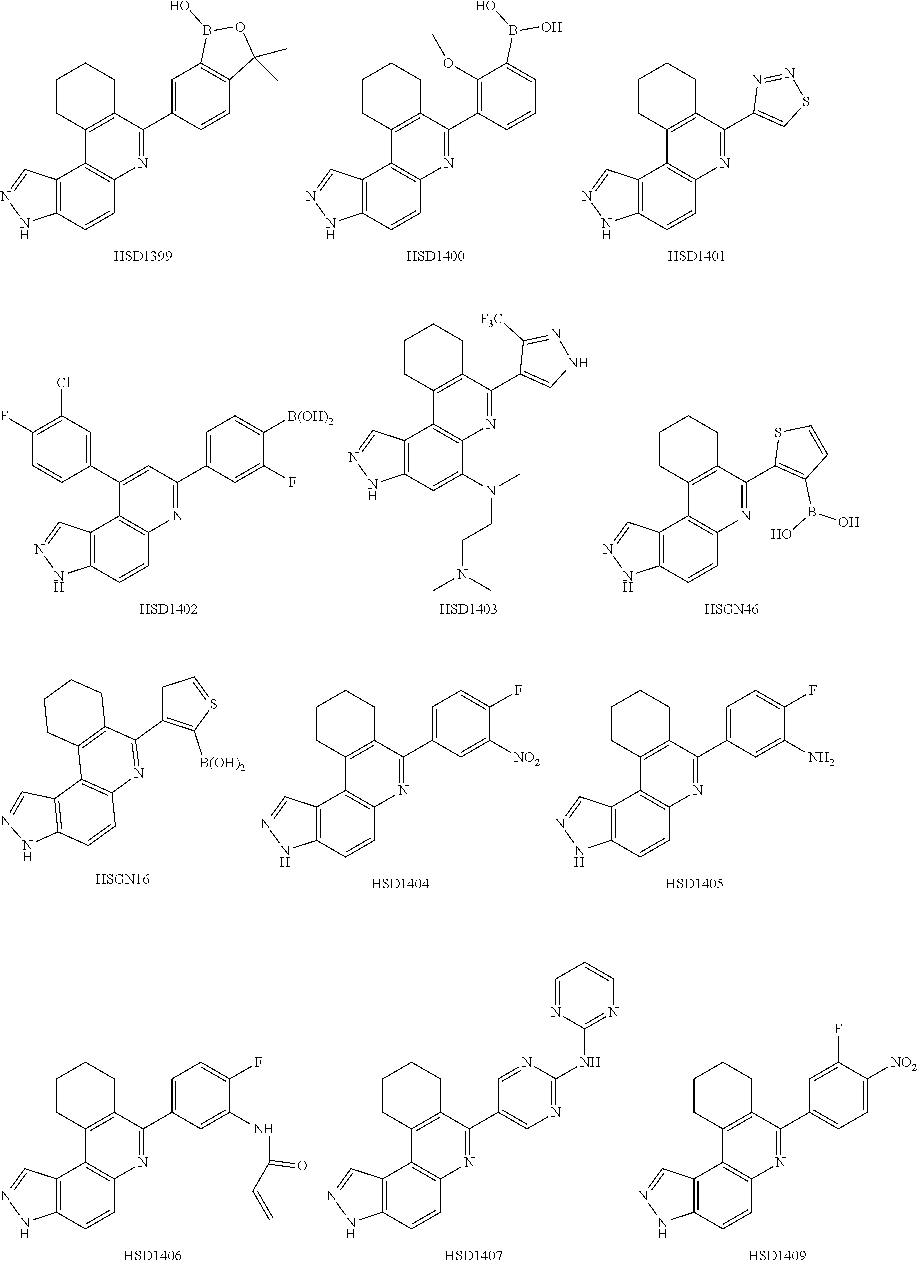

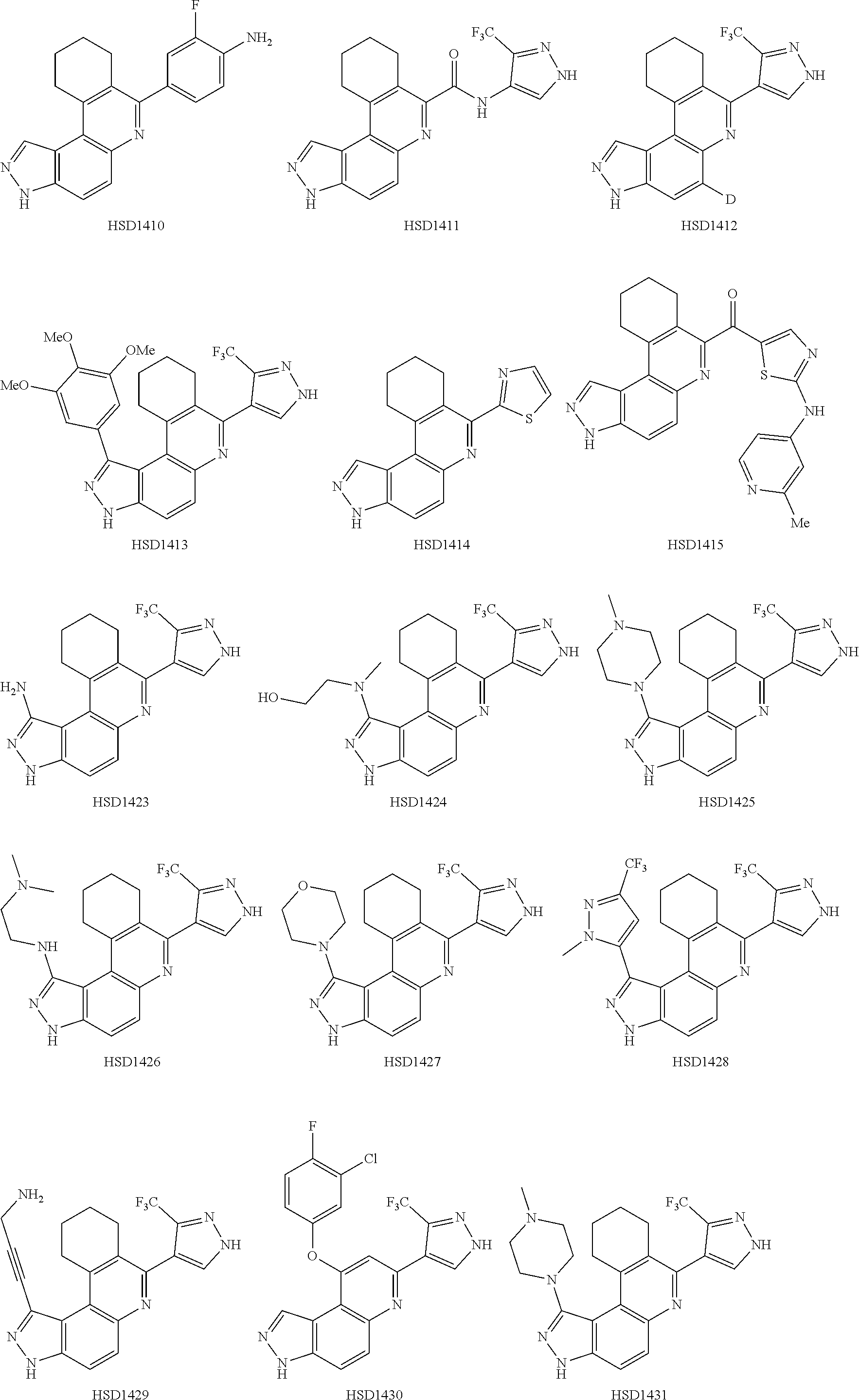

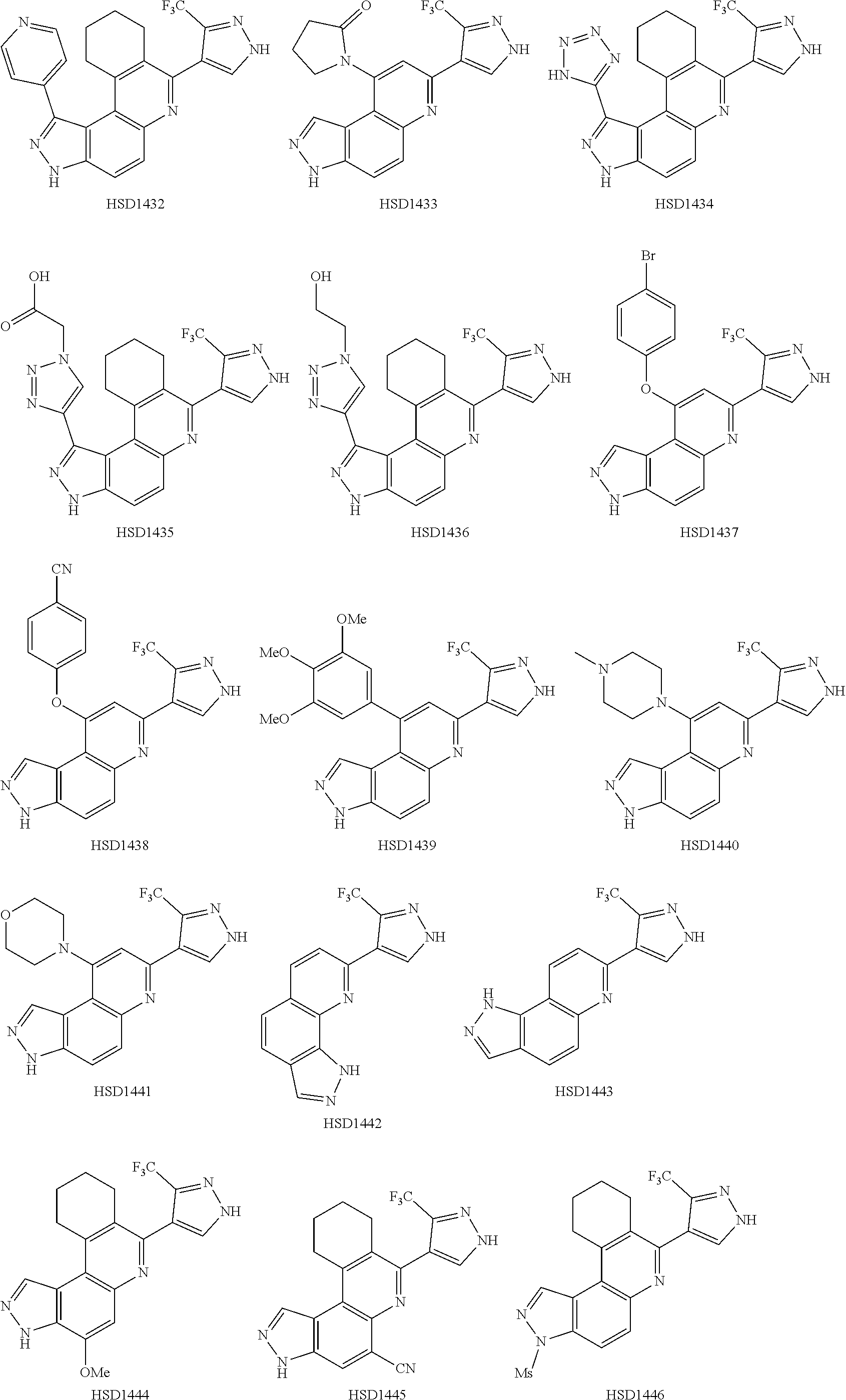

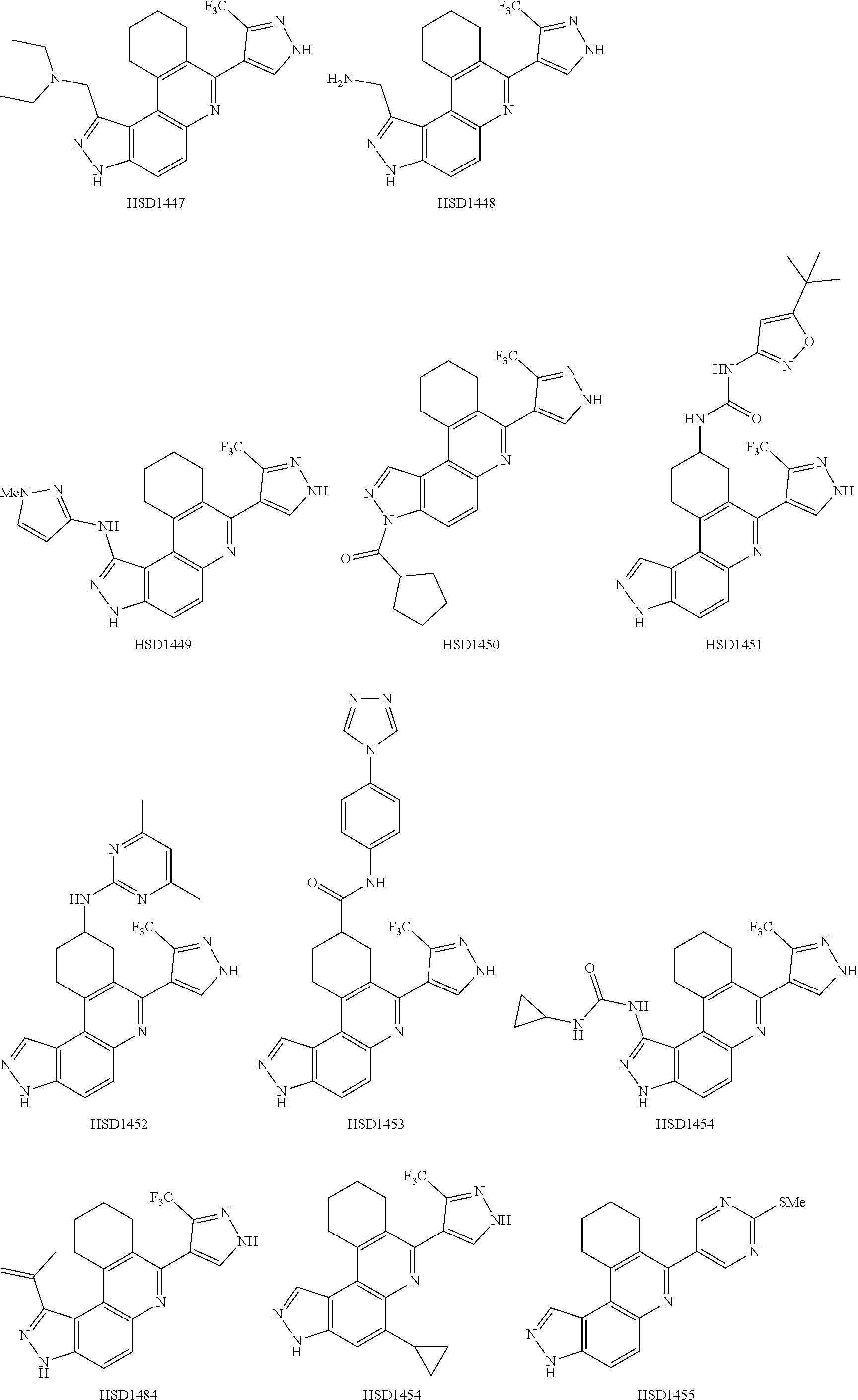

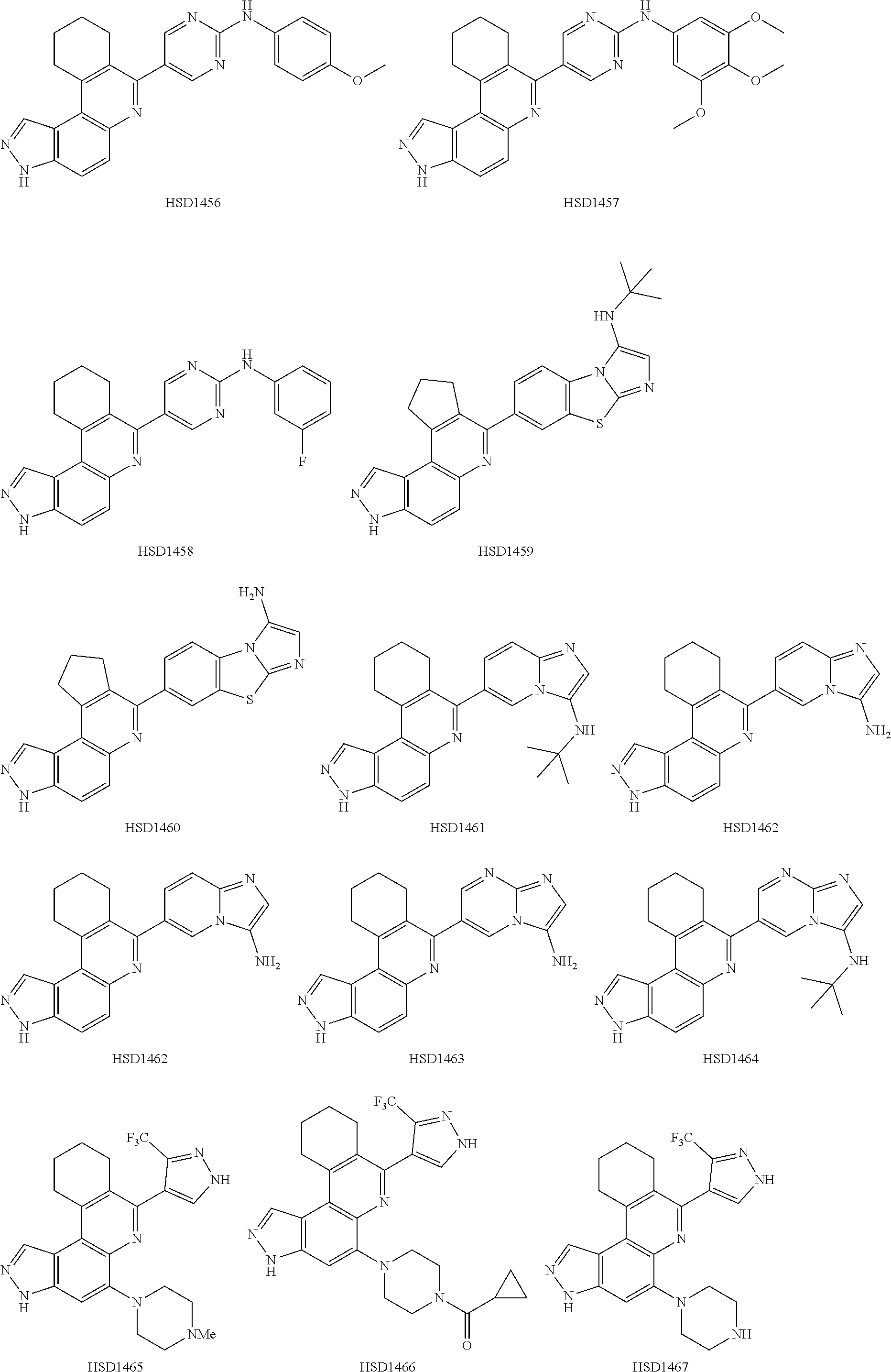

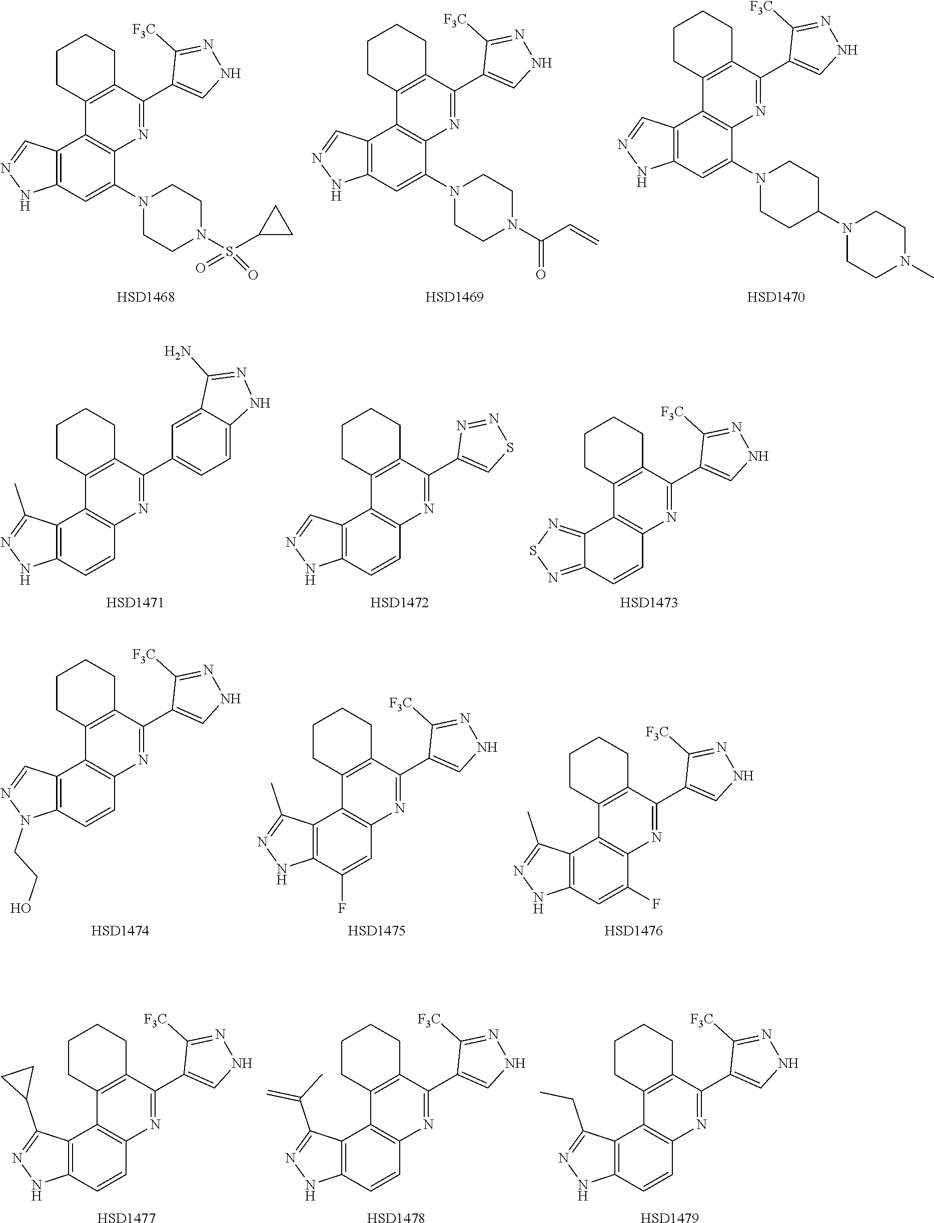

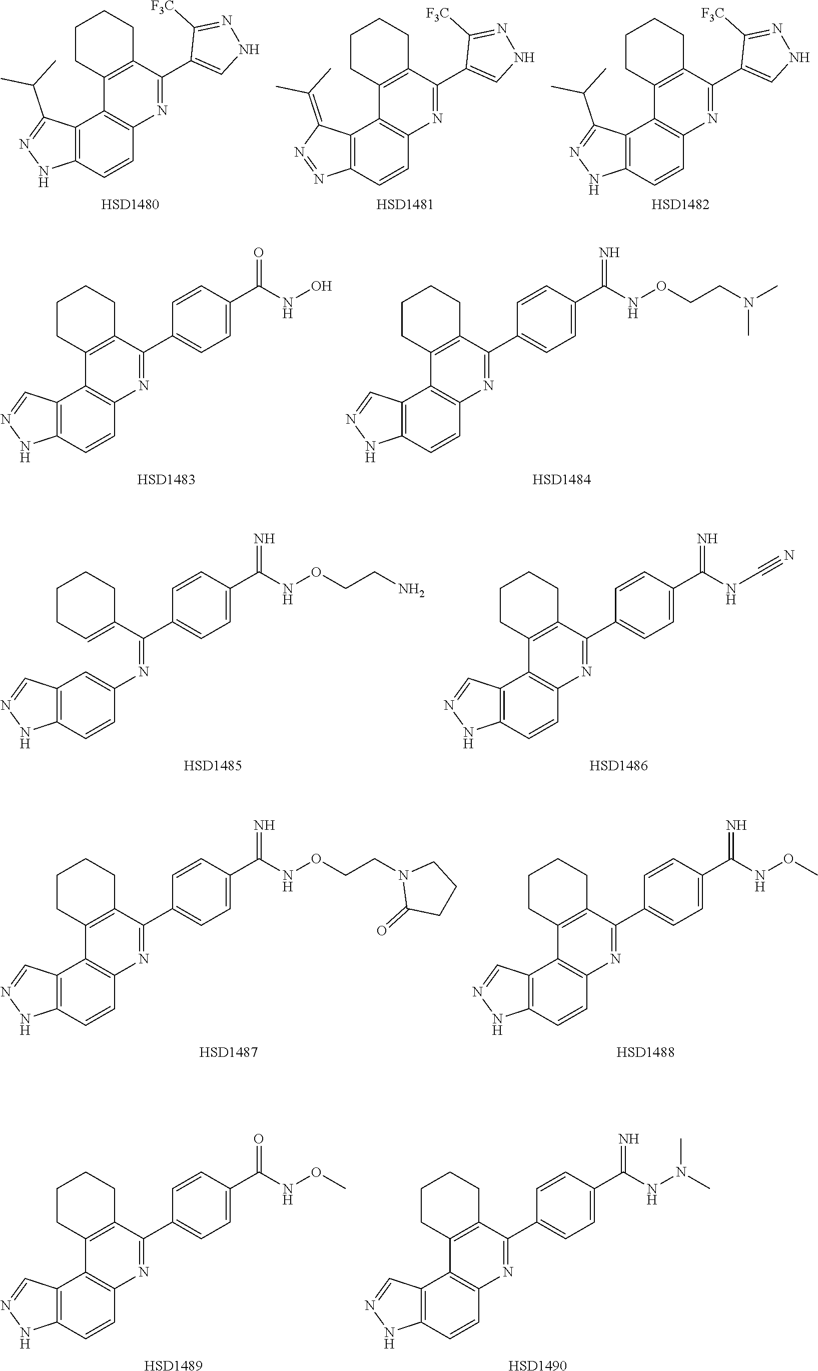

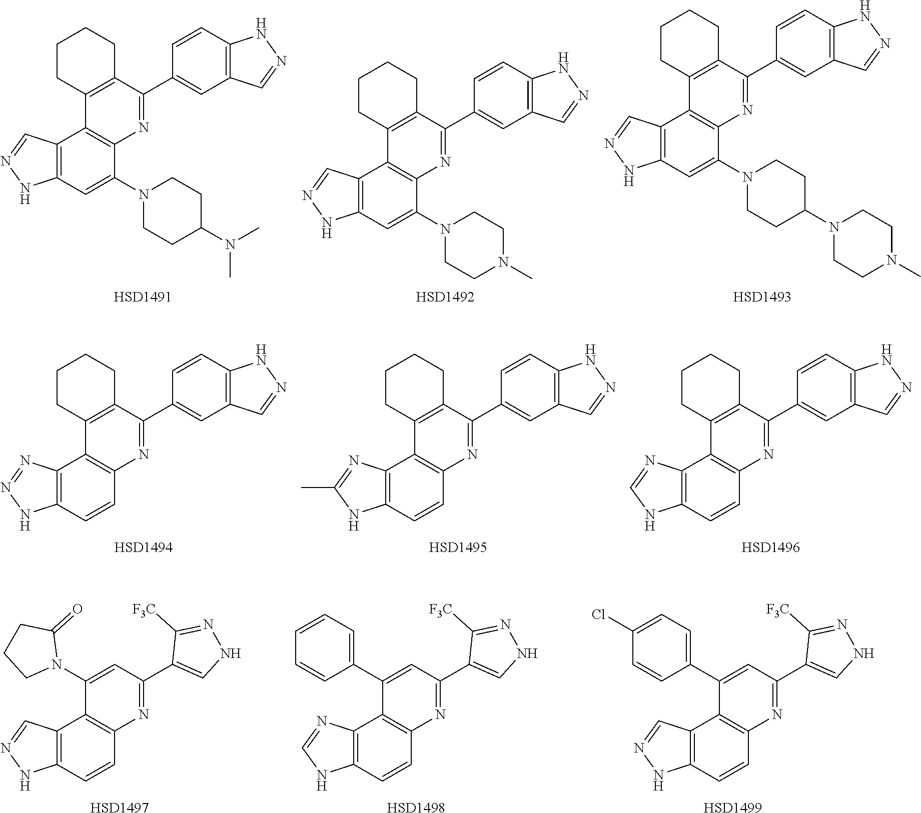

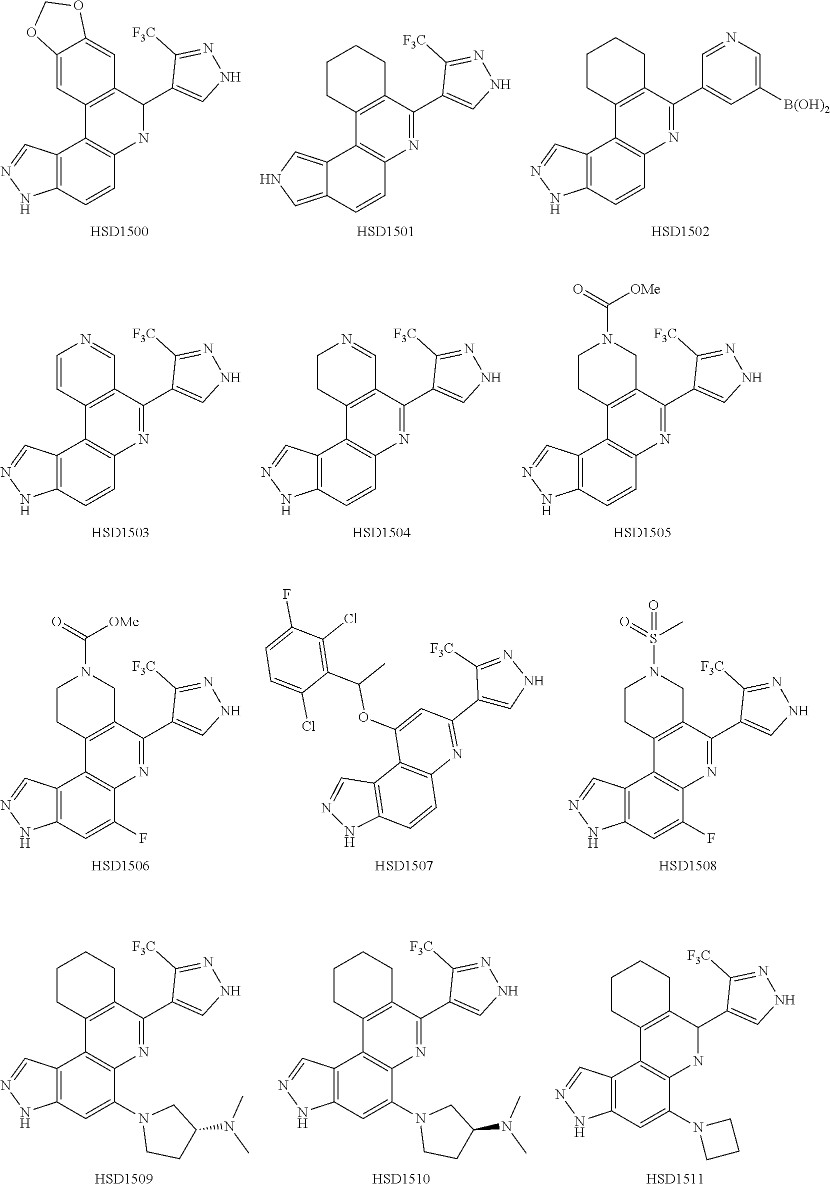

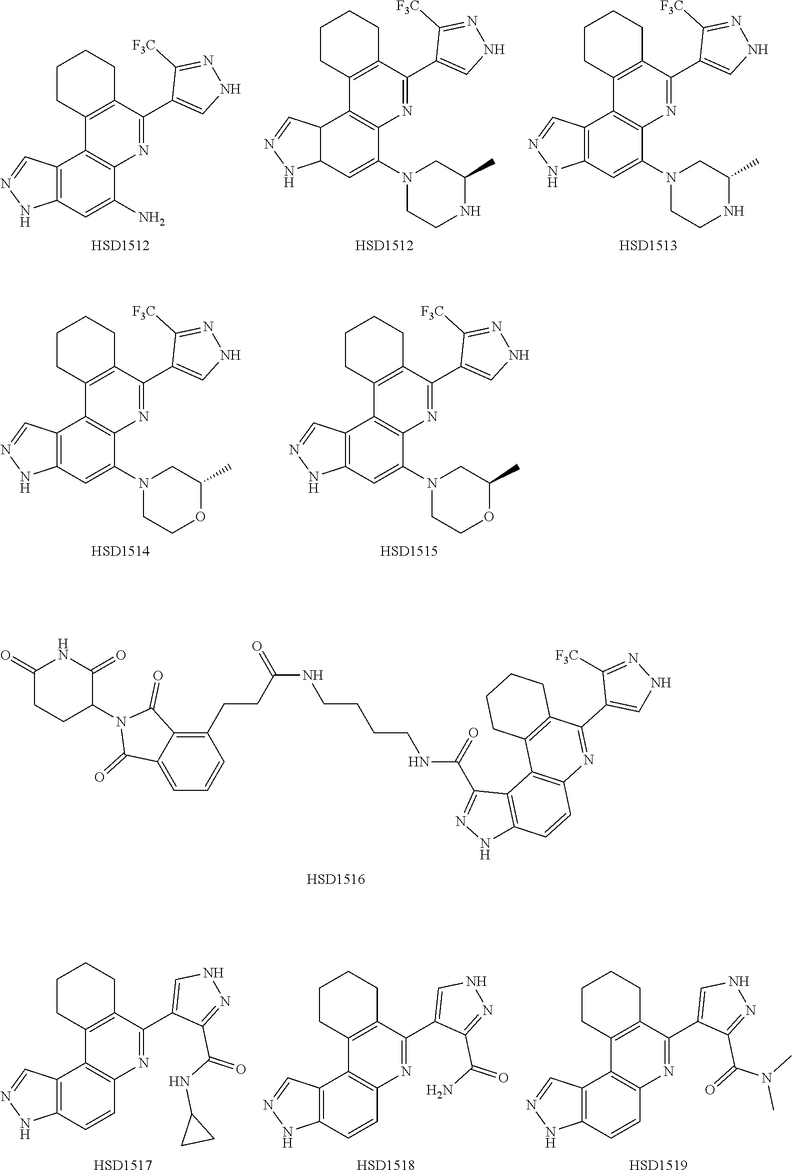

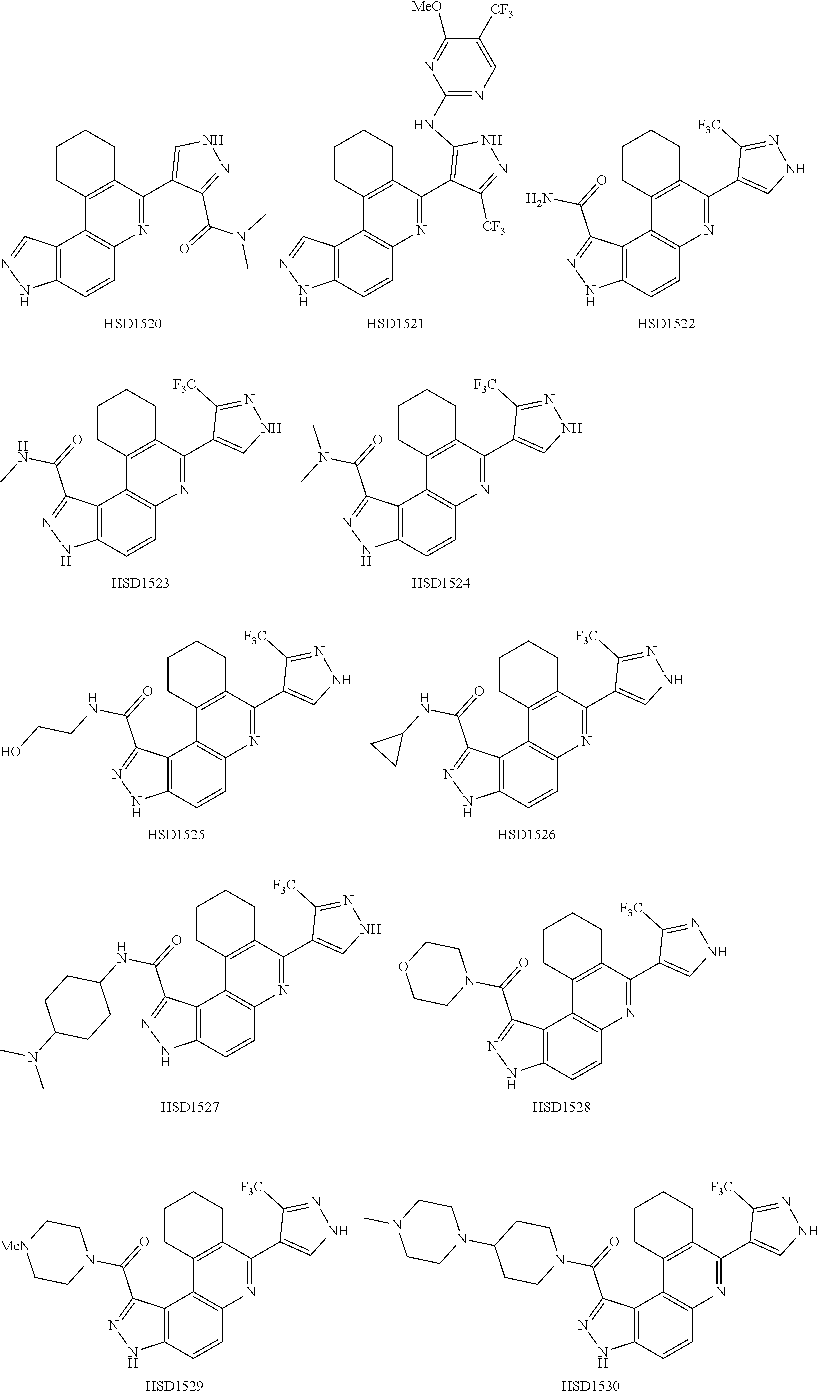

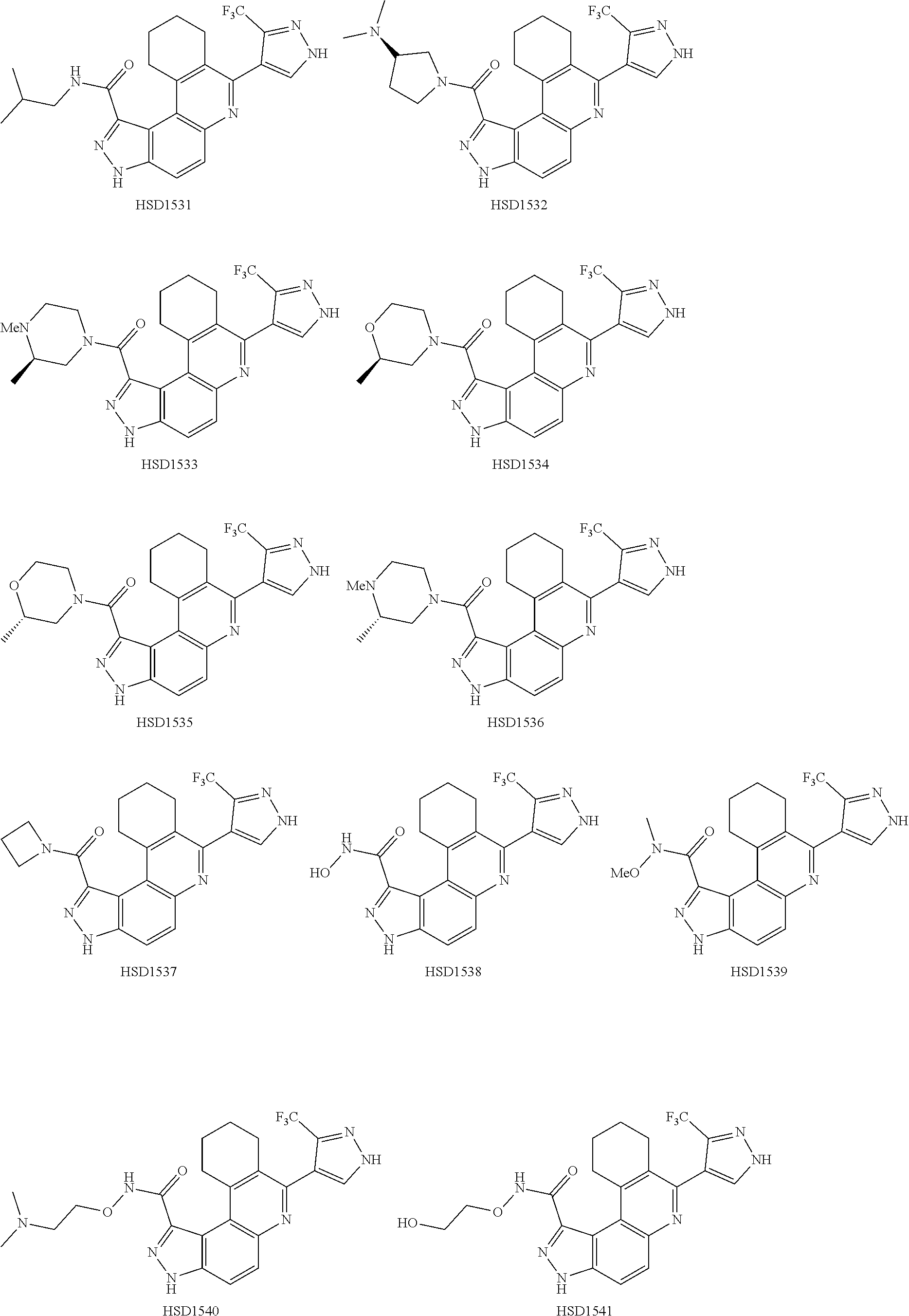

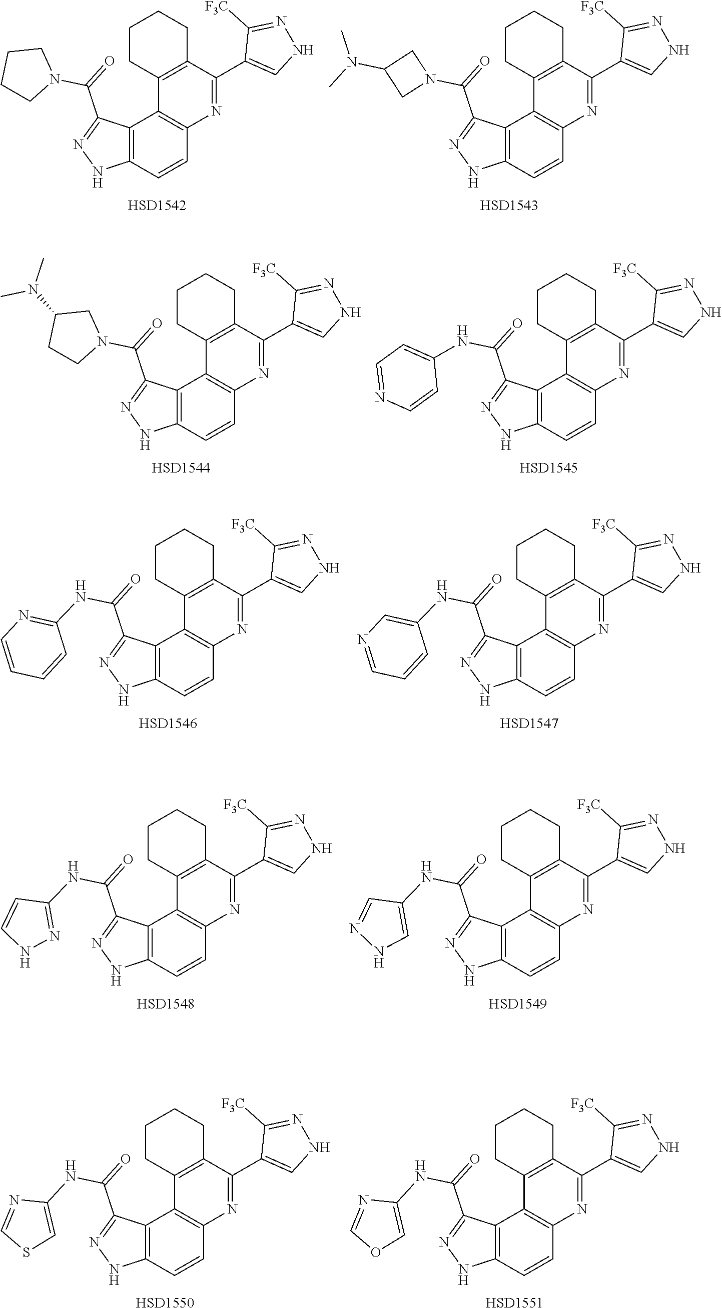

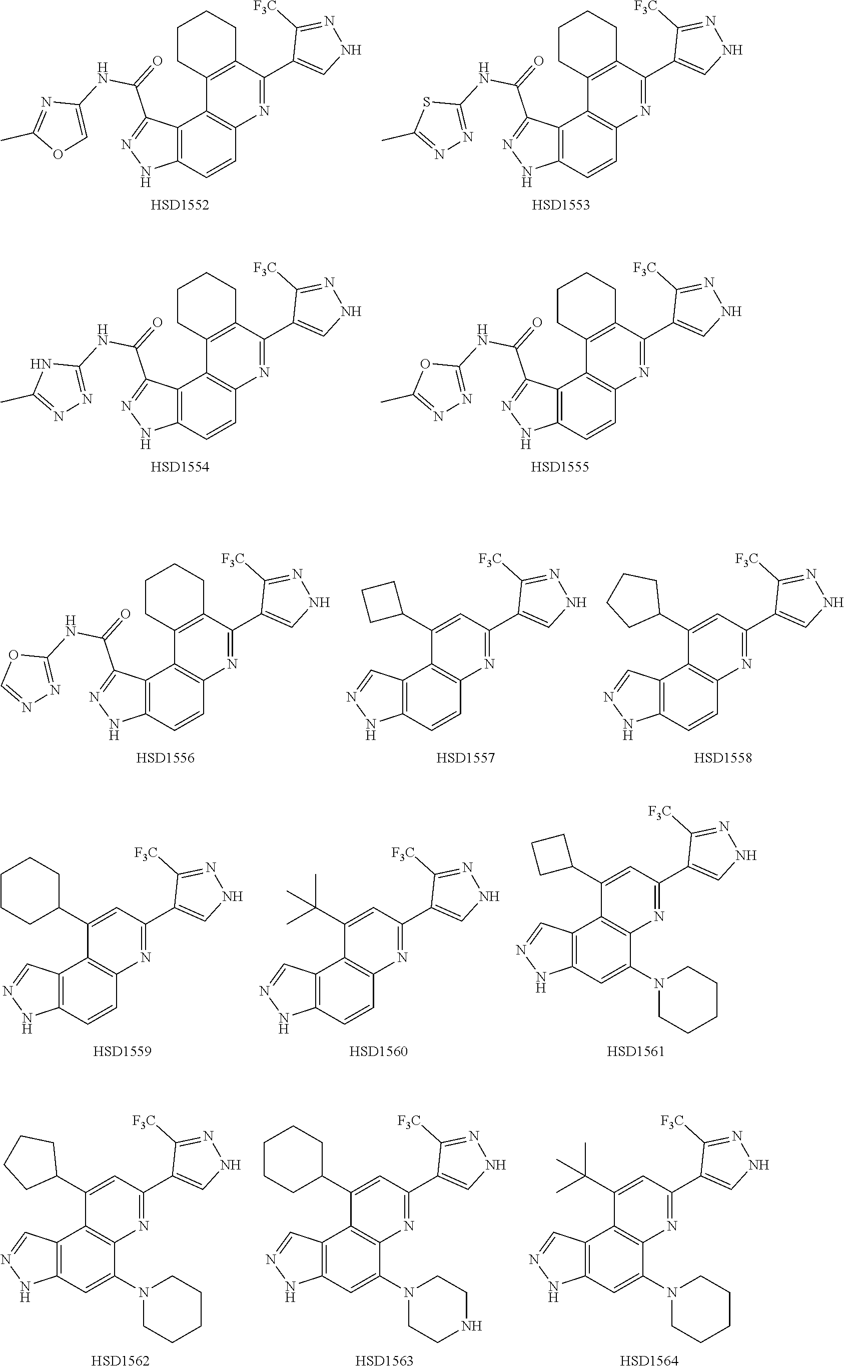

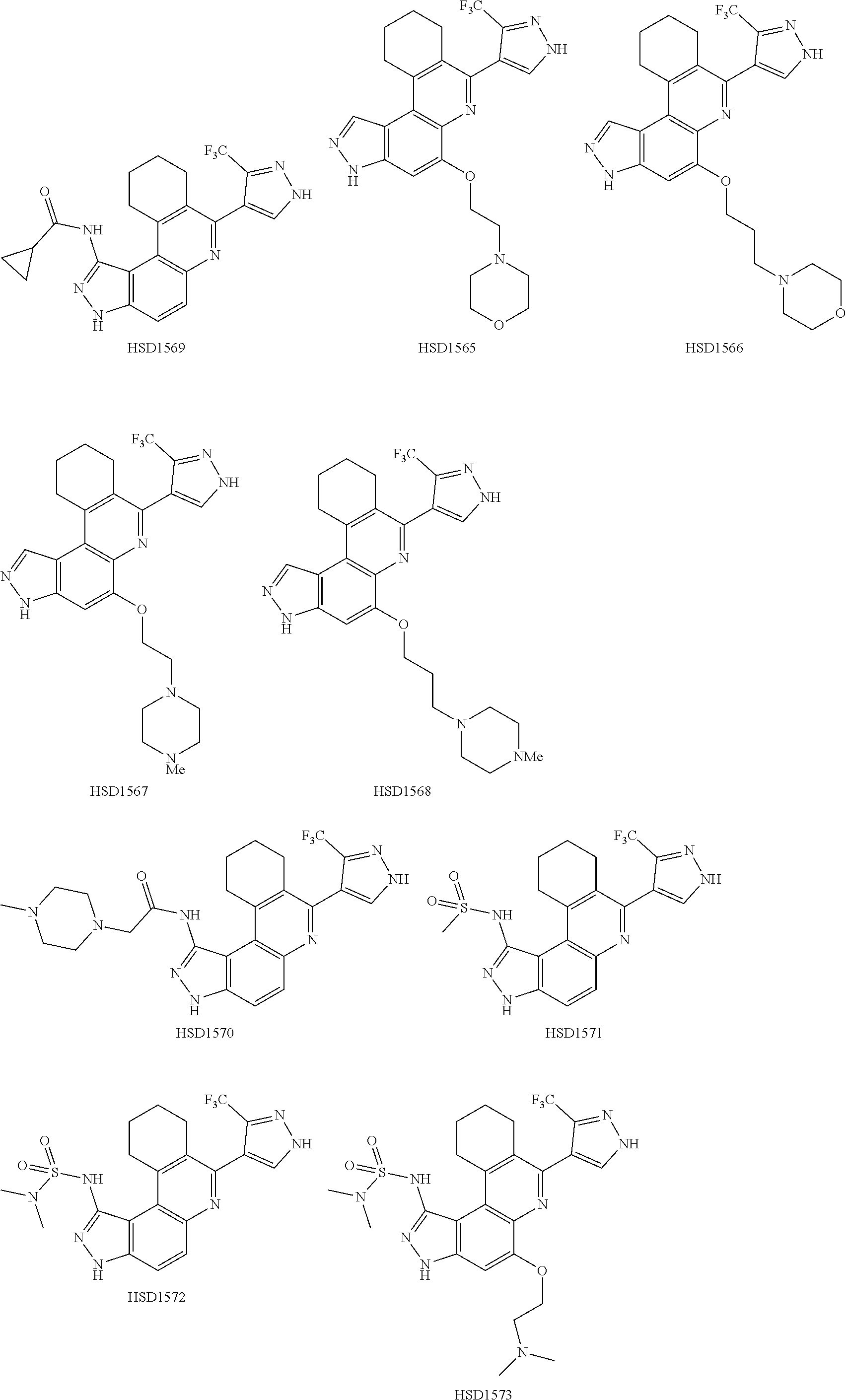

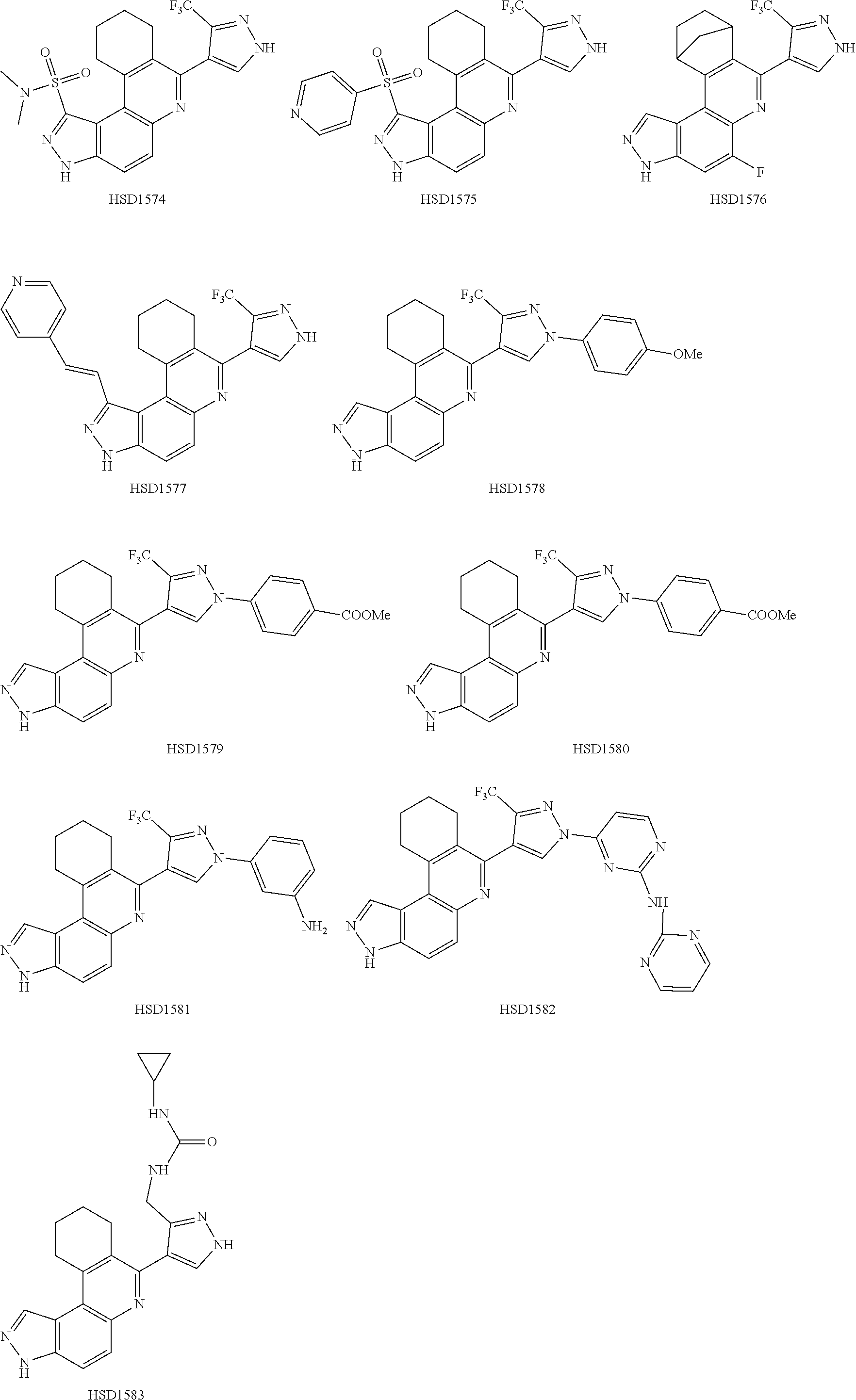

In some illustrative embodiments, the present invention relates to a pharmaceutical composition comprising one or more compounds disclosed herein, or a pharmaceutically acceptable salt thereof, together with one or more diluents, excipients or carriers.

In some illustrative embodiments, the present invention relates to a kinase inhibitor, wherein the kinase is selected from the group consisting of FLT3, MNK1/2, JAK1/2/3, Limk1/2, various CDKs, Haspin, ROCK1/2, TOPK, LRRK2, GSK3a/3b, RSK1-4, ERK, P70S6K, AKT, PI3K, p38, PKC, PKA, FGFR1-4, VEGFR1-3, ALK, AXL, LIMK1/2, Aurora A/B, ABL1, AKT, CSF1R, CSNK1D, DCAMKL1, CSNK1G2, EPHA2, ERBB2, IKK-alpha, IKK-beta, JNK1/2/3, MARK3, MEK1/2, MET, MLK1, PAK1/2/4, PDGFRa/b, PIM1/2/3, PLK1/2/3/4, PRKCE, PRKX, RET, TAOK2, TRKA/B/C, ULK2, and receptor-interacting protein kinase 4 (RIPK4).

In some illustrative embodiments, the present invention relates to a method for treating diseases mediated by a kinase and/or histone demethylases, including inflammation, cancer, viral and bacterial infections, gastrointestinal disorders, eye diseases, neurological, cardiovascular and immunological disorders, comprising the step of administering a therapeutically effective amount of one or more compounds disclosed herein, and one or more carriers, diluents, or excipients, to a patient in need of relief from said cancer.

In some illustrative embodiments, the present invention relates to a method for treating diseases mediated by a kinase and histone demethylases, including inflammation, cancer, viral and bacterial infections, gastrointestinal disorders, eye diseases, neurological, cardiovascular and immunological disorders, comprising the step of administering a therapeutically effective amount of a compound disclosed herein in combination with one or more other compounds of the same or different mode of action, and one or more carriers, diluents, or excipients, to a patient in need of relief from said cancer.

In some other illustrative embodiments, the present invention relates to a drug conjugate, either small molecule or biologic conjugate, comprising one or more compounds disclosed herein, wherein the conjugate confers cell-type or tissue type targeting or the conjugate targets another pathway that synergies the action of the compounds.

In some illustrative embodiments, the present invention relates to a drug conjugate, either small molecule or biologic conjugate, comprising one or more compounds disclosed herein, wherein the conjugate confers aqueous solubility or low clearance.

In some illustrative embodiments, the present invention relates to a drug conjugate containing one or more compounds disclosed herein, and a moiety that aids the degradation of target proteins via systems including but not limited to the ubiquitin ligase/proteosome degradation system.

In some illustrative embodiments, the present invention relates to a pharmaceutical composition comprising nanoparticles of one or more compounds disclosed herein, together with one or more diluents, excipients or carriers.

In some illustrative embodiments, the present invention relates to a prodrug comprising one or more compounds disclosed herein, wherein the prodrug moiety is removed at specific location, such as gastrointestinal or in blood or in tissues or in cancer specific.

In some illustrative embodiments, the present invention relates to an analogs of compounds disclosed herein whereby specific metabolic hot spots are modified with groups such as deuterium or fluorine.

In addition, it is appreciated herein that the compounds described herein may be used in combination with other compounds that are administered to treat other symptoms of cancer, such as compounds administered to relieve pain, nausea, vomiting, and the like.

The following non-limiting exemplary embodiments are included herein to further illustrate the invention. These exemplary embodiments are not intended and should not be interpreted to limit the scope of the invention in any way. It is also to be understood that numerous variations of these exemplary embodiments are contemplated herein.

### Experimental Section and Characterization:

### General Procedure for the Multicomponent Reaction:¹⁰

Method A: A mixture of amine (1 mmol) and aldehyde (1 mmol) in 3 mL of absolute ethanol was refluxed for 2 h followed by addition of cyclic ketone or acetaldehyde (2.1 mmol) to the reaction mixture. A catalytic amount of cone, hydrochloric acid was added and the reaction was continued to reflux for 6-12 h. Reaction mixture concentrated and dissolved in DCM (50 mL), washed with brine solution (20 mL×2). The organic layer was dried (Na₂SO₄), concentrated under reduced pressure, and purified by silica gel chromatography (dichloromethane:methanol (99:01 to 80:20) to give the desired cyclized compound.

Method B: A mixture of amine (1 mmol) and aldehyde (1 mmol) in 3 mL acetonitrile was refluxed for 2 h. The reaction mixture allowed to cool to room temperature followed by addition of alkene (2 mmol) and Y(OTf)₃ (30 mol %). The reaction continued to reflux for overnight. Reaction mixture concentrated and purified by silica gel chromatography dichloromethane:methanol (99:01) to give the desired cyclized compound.

Method C: A mixture of amine (1 mmol), aldehyde (1 mmol) and cyclic ketone (2 mmol) in 6 mL tetrahydrofuran in presence of iodine (10 mol %) was refluxed for 6-12 h. After completion of reaction, reaction mixture was concentrated and purified by silica gel chromatography ethyl acetate:hexane (80:20) or dichloromethane:methanol (99:01) to give the desired cyclized compound. (Note: Sometime product may get precipitated out, which was filtered, washed with absolute ethanol and further purified by column chromatography).

Kinase assay: HotSpot kinase screening assay (Reaction Biology) was used to measure kinase/inhibitor interactions. Kinase and substrate were mixed in a buffer containing 20 mM HEPES pH 7.5, 10 mM MgCl₂, 1 mM EGTA, 0.02% Brij35, 0.02 mg/mL BSA, 0.1 mM Na₃VO₄, 2 mM DTT and 1% DMSO. Single-dose of compounds (500 nM) were then added to each reaction mixture. After 20-minute incubation, ATP (Sigma) and [γ−³³P] ATP (Perkin Elmer) were added at a final total concentration of 100 μM for addition 2 hours at room temperature, followed by spotting onto P81 ion exchange cellulose chromatography paper (Whatman, Inc.).

Filter paper was washed in 0.75% phosphoric acid to remove unincorporated ATP. Percent remaining kinase activity of a vehicle (DMSO) containing kinase reaction was calculated for each kinase/inhibitor pair using Prism 5 (GraphPad).

### LSD1 Assay:

The LSD1 Assay Kit was used to measure LSD1 activity of purified LSD1 enzyme. In a 96 well plate was added LSD1 buffer, 10 μM Histone H3(1-21)K4mc2 peptide, and the test compound or DMSO control. The reaction was initiated with LSD1 enzyme. After 30 min incubation at room temperature, peroxidase and Amplex Red reagents are added and fluorescence (λex=530±13 nm. λ cm=590±18 nm) is measured after 5 min, using a plate reader. Percentage inhibition is calculated as fluorescence intensity in the presence of inhibitor divided by DMSO control times 100%.

### IC₅₀ Proliferation Assay

Cell lines and primary cells were seeded into 96-well plates the afternoon prior to treatment. Approximately 18 hours later, compounds were semi-serially diluted in dimethyl sulfoxide (DMSO) and then growth medium, and added to cells. Plates were incubated for 72 hours prior to addition of Alamar Blue (Life Technologies. Carlsbad, CA). Plates were read after 4 additional hours of incubation at 37° C. using a Bio-Tek Synergy HT plate reader (Bio-Tek, Winooski. VT). Data was analyzed and graphed using GraphPad Prism Software (Graphpad, La Jolla. CA).

### 4-(8,9,10,11-Tetrahydro-3H-pyrrolo[3,2-a]phenanthridin-7-yl)phenol

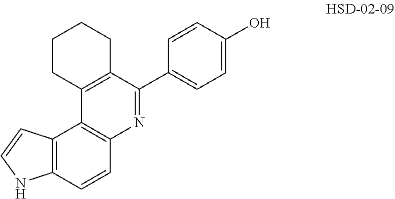 [SMILES: Oc1ccc(-c2nc3ccc4[nH]ccc4c3c3c2CCCC3)cc1]

Yellow solid (195 mg, 62%). ¹H NMR (500 MHz, DMSO-d₆) δ 10.35 (s, 1H), 8.20 (d, J=9.0 Hz, 1H), 8.02 (d, J=9.0 Hz, 1H), 7.79 (s, 1H), 7.58 (d, J=8.2 Hz, 2H), 7.31 (s, 1H), 7.05 (d, J=8.2 Hz, 2H), 3.56 (d, J=6.6 Hz, 2H), 2.80 (t, J=6.3 Hz, 2H), 2.02 (d, J=8.4 Hz, 2H), 1.87-1.61 (m, 2H), ¹³C NMR (126 MHz, DMSO-d₆) δ 160.28, 150.26, 133.77, 131.92, 129.82, 127.07, 123.08, 121.33, 119.87, 115.98, 106.80, 31.35, 28.22, 22.01, 21.65; HRMS (ESI) m/z calcd for C₂₁H₁₉N₂O [M+H]⁺ 315.1497, found 315.1499.

### 4-(8,9,10,11-Tetrahydro-3H-pyrazolo[4,3-a]phenanthridin-7-yl)phenol

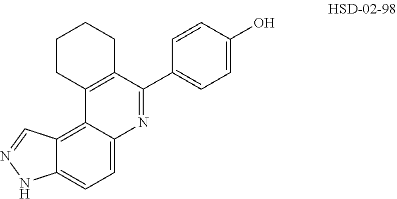 [SMILES: Oc1ccc(-c2nc3ccc4[nH]ncc4c3c3c2CCCC3)cc1]

Method A: Off white solid (189 mg, 60%). ¹H NMR (500 MHz, DMSO-d₆) δ 10.46 (s, 1H), 8.67 (s, 1H), 8.16 (d, J=9.5 Hz, 1H), 7.60 (d, J=8.1 Hz, 2H), 7.06 (d, J=5 Hz, 1H), 3.31 (s, 1H), 2.80 (s, 1H), 2.00 (s, 1H), 1.74 (s, 1H); ¹³C NMR (126 MHz, DMSO-d₆) δ 160.40, 152.79, 151.09, 134.91, 132.09, 131.87, 122.67, 122.43, 119.96, 115.95, 114.66, 30.85, 28.21, 21.72, 21.66; HRMS (ESI) m/z calcd for C₂₀H₁₈N₃O [M+H]⁺ 316.1449, found 316.1458.

### 4-(8,9-Dihydro-3H-cyclobuta[c]pyrrolo[3,2-f]quinolin-7-yl)phenol

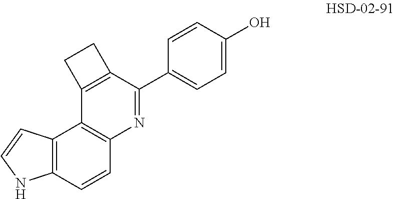 [SMILES: Oc1ccc(-c2nc3ccc4[nH]ccc4c3c3c2CC3)cc1]

Method C: Yellow solid (166 mg, 58%). ¹H NMR (500 MHz, Methanol-d₄) δ 8.12 (d, J=9.1 Hz, 1H), 8.00 (d, J=8.5 Hz, 2H), 7.97 (d, J=9.2 Hz, 2H), 7.65 (d, J=3.0 Hz, 1H), 7.14-7.04 (m, 2H), 3.92 (t, J=3.7 Hz, 2H), 3.85 (t, J=3.8 Hz, 2H); ¹³C NMR (126 MHz, DMSO) δ 161.86, 154.45, 143.71, 135.70, 134.66, 132.88, 131.33, 127.84, 123.68, 120.93, 119.32, 118.50, 116.87, 109.93, 103.31, 31.49. HRMS (ESI) m/z calcd for C₁₉H₁₅N₂O [M+H]⁺ 287.1184, found 287.1184.

### 4-(3,8,9,10-Tetrahydrocyclopenta[c]pyrrolo[3,2-f]quinolin-7-yl)phenol

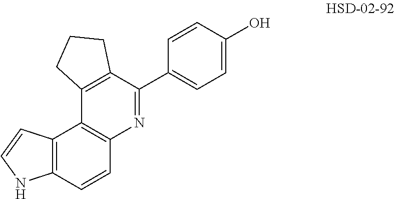 [SMILES: Oc1ccc(-c2nc3ccc4[nH]ccc4c3c3c2CCC3)cc1]

Method A: Yellow solid (181 mg, 60%). ¹H NMR (500 MHz, Methanol-d₄) δ 8.18 (dd, J=9.0, 0.9 Hz, 1H), 7.92 (d, J=9.0 Hz, 1H), 7.79-7.72 (m, 2H), 7.70 (d, J=3.1 Hz, 1H), 7.27 (dd, J=3.1, 0.9 Hz, 1H), 7.11-7.04 (m, 2H), 3.92-3.79 (m, 2H), 3.37 (t, J=7.6 Hz, 2H), 2.49 (p, J=7.7 Hz, 2H); ¹³C NMR (126 MHz, Methanol-d₄) δ 160.89, 146.91, 136.04, 135.25, 133.22, 130.80, 126.63, 122.44, 121.08, 120.85, 120.21, 115.78, 112.16, 104.20, 35.16, 31.66, 24.38; HRMS (ESI) m/z calcd for C₂₀H₁₇N₂O [M+H]⁺ 301.1340, found 301.1340.

### 4-(9-Methyl-8,9,10,11-tetrahydro-3H-pyrrolo[3,2-a]phenanthridin-7-yl)phenol

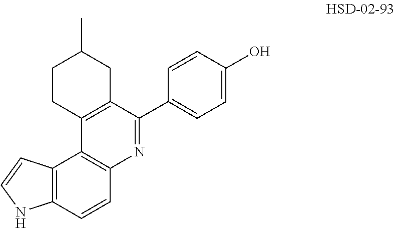 [SMILES: CC1CCc2c(c(-c3ccc(O)cc3)nc3ccc4[nH]ccc4c23)C1]

Method A: Yellow solid (236 mg, 72%). ¹H NMR (500 MHz, Methanol-d₄) δ 8.17 (d, J=8.9 Hz, 1H), 7.84 (d, J=8.8 Hz, 1H), 7.71 (d, J=3.1 Hz, 1H), 7.55 (d, J=8.6 Hz, 2H), 7.40 (d, J=3.2 Hz, 1H), 7.07 (d, J=8.6 Hz, 2H), 3.86 (ddd, J=19.3, 5.9, 2.4 Hz, 1H), 3.69-3.50 (m, 1H), 2.89 (ddd, J=16.7, 4.5, 2.0 Hz, 11K), 2.62 (dd, J=16.9, 10.6 Hz, 1H), 2.30 (ddt, J=13.0, 7.0, 2.4 Hz, 1H), 1.98-1.83 (m, 1H), 1.71 (dtd, J=12.9, 10.9, 5.8 Hz, 1H), 1.14 (d, J=6.5 Hz, 3H); ¹³C NMR (126 MHz, Methanol-d₄) δ 160.12, 154.73, 150.23, 133.79, 130.78, 129.45, 126.06, 123.12, 122.70, 120.75, 120.16, 115.52, 112.48, 106.36, 36.01, 31.36, 29.62, 27.78, 20.23; HRMS (ESI) m/z calcd for C₂₂H₂₁N₂O [M+H]⁺ 329.1653, found 329.1653.

### 2,6-Dibromo-4-(8,9,10,11-tetrahydro-3H-pyrrolo[3,2-a]phenanthridin-7-yl)phenol

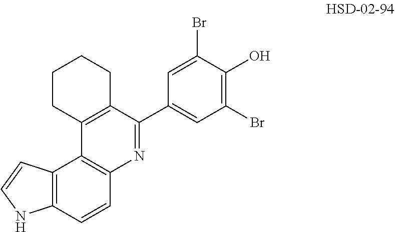 [SMILES: Oc1c(Br)cc(-c2nc3ccc4[nH]ccc4c3c3c2CCCC3)cc1Br]

Method A: Yellow solid (277 mg, 59%). ¹H NMR (500 MHz, DMSO-d₆) δ 11.80 (s, 1H), 10.10 (s, 1H), 7.80 (d, J=8.9 Hz, 0H), 7.73 (s, 1H), 7.64 (d, J=8.9 Hz, 0H), 7.51 (t, J=2.7 Hz, 0H), 7.13 (t, J=2.5 Hz, 1H), 3.37 (t, J=6.5 Hz, 1H), 2.79 (t, J=6.1 Hz, 1H), 2.03-1.89 (m, 1H), 1.74 (dq, J=6.3, 3.2 Hz, 1H); ¹³C NMR (126 MHz, DMSO) δ 152.61, 150.86, 142.79, 135.62, 133.32, 127.69, 124.22, 123.48, 122.70, 120.25, 116.95, 116.65, 111.87, 106.37, 30.09, 28.90, 22.72, 22.48; HRMS (ESI) m/z calcd for C₂₁H₁₇Br₂N₂O [M+H]⁺ 470.9707, found 470.9705.

### 7-(4-Bromophenyl)-8,9,10,11-tetrahydro-3H-pyrrolo[3,2-a]phenanthridine

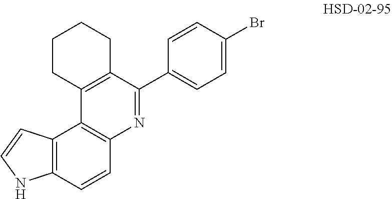 [SMILES: Brc1ccc(-c2nc3ccc4[nH]ccc4c3c3c2CCCC3)cc1]

Method A: Off white solid (207 mg, 55%). ¹H NMR (500 MHz, Methanol-d₄) δ 7.80 (dd, J=8.9, 0.9 Hz, 1H), 7.70 (d, J=8.9 Hz, 1H), 7.66 (d, J=8.4 Hz, 2H), 7.44-7.40 (m, 3H), 7.20 (dd, J=3.3, 0.9 Hz, 1H), 3.53-3.45 (m, 2H), 2.74 (t, J=6.2 Hz, 2H), 2.13-2.01 (m, 2H), 1.87-1.76 (m, 2H); ¹³C NMR (126 MHz, Methanol-d₄) δ 154.80, 143.92, 142.60, 140.03, 133.30, 131.01, 130.67, 127.22, 122.92, 122.85, 121.94, 121.71, 120.23, 116.08, 105.79, 29.93, 28.51, 22.43, 22.09; HRMS (ESI) m/z calcd for C₂₁H₁₈BrN₂ [M+H]⁺ 377.0653, found 377.0653.

### 4-(9-Methyl-3H-pyrrolo[3,2-f]quinolin-7-yl)phenol

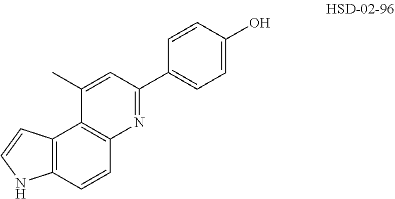 [SMILES: Cc1cc(-c2ccc(O)cc2)nc2ccc3[nH]ccc3c12]

Method A: Yellow solid (173 mg, 63%). ¹H NMR (500 MHz, DMSO-d₆) δ 10.57 (s, 1H), 8.24 (d, J=6.4 Hz, 3H), 8.09 (d, J=8.8 Hz, 1H), 7.80 (s, 0H), 7.28 (s, 1H), 7.07 (d, J=8.8 Hz, 1H), 3.14 (s, 3H), ¹³C NMR (126 MHz, DMSO-d₆) δ 161.75, 149.00, 133.54, 131.40, 127.61, 122.28, 121.80, 121.28, 120.94, 116.69, 105.84, 24.33; HRMS (ESI) m/z calcd for C₁₈H₁₅N₂O [M+H]⁺275.1184, found 275.1192.

### 7-(3,4,5-Trimethoxyphenyl)-8,9,10,11-tetrahydro-3H-pyrrolo[3,2-a]phenanthridine

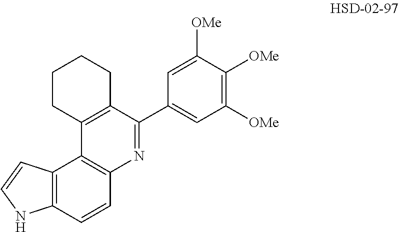 [SMILES: COc1cc(-c2nc3ccc4[nH]ccc4c3c3c2CCCC3)cc(OC)c1OC]

Method A: White crystalline solid (213 mg, 55%). ¹H NMR (500 MHz, Methanol-d₄) δ 8.18 (dd, J=8.9, 0.8 Hz, 1H), 7.85 (d, J=8.9 Hz, 1H), 7.71 (d, J=3.1 Hz, 1H), 7.38 (dd, J=3.2, 0.9 Hz, 1H), 7.01 (s, 2H), 3.92 (s, 6H), 3.88 (s, 3H), 3.67 (t, J=6.6 Hz, 3H), 2.93 (t, J=6.2 Hz, 2H), 2.22-2.11 (m, 3H), 1.92 (ddd, J=9.1, 7.4, 4.5 Hz, 2H), ¹³C NMR (126 MHz, Methanol-d₄) δ 154.94, 153.66, 149.75, 139.80, 133.79, 133.67, 129.57, 127.67, 126.01, 123.58, 120.83, 120.04, 112.80, 106.71, 106.41, 59.88, 55.66, 31.23, 27.71, 21.56, 21.24; HRMS (ESI) m/z calcd for C₂₄H₂₅N₂O₃ [M+H]⁺ 389.1865, found 389.1861.

### 4-(9,9-dimethyl-8,9,10,11-tetrahydro-3H-pyrrolo[3,2-a]phenanthridin-7-yl)phenol

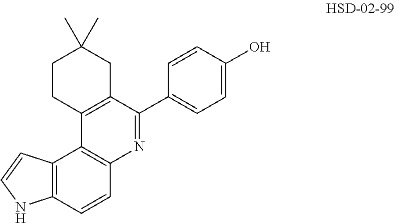 [SMILES: CC1(C)CCc2c(c(-c3ccc(O)cc3)nc3ccc4[nH]ccc4c23)C1]

Method A: White crystalline solid (188 mg, 53%). ¹H NMR (500 MHz, Methanol-d₄) δ 7.79 (d, J=8.9 Hz, 1H), 7.71 (d, J=8.9 Hz, 1H), 7.43 (d, J=3.1 Hz, 1H), 7.32 (d, J=8.1 Hz, 2H), 7.21 (d, J=3.1 Hz, 1H), 6.92 (d, J=8.1 Hz, 2H), 3.50 (t, J=6.8 Hz, 2H), 2.56 (s, 2H), 1.80 (t, J=6.7 Hz, 2H), 0.97 (s, 6H); ¹³C NMR (126 MHz, Methanol-d₄) δ 157.35, 156.02, 143.16, 141.88, 133.19, 131.52, 130.07, 126.84, 122.96, 122.26, 121.28, 120.30, 116.11, 114.60, 105.73, 48.11, 48.05, 47.94, 47.77, 47.60, 47.43, 47.26, 47.09, 42.06, 34.69, 28.29, 27.71, 26.60; HRMS (ESI) m/z calcd for C₂₃H₂₃N₂O [M+H]⁺ 343.1810, found 343.1808.

### 5-(8,9,10,11-Tetrahydro-3H-pyrrolo[3,2-a]phenanthridin-7-yl)benzene-1,2,3-triol

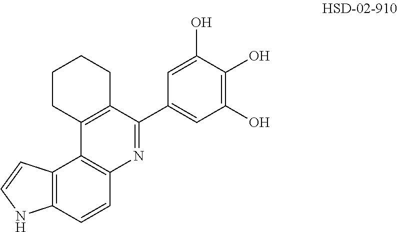 [SMILES: Oc1cc(-c2nc3ccc4[nH]ccc4c3c3c2CCCC3)cc(O)c1O]

Method A: Yellow solid (246 mg, 71%). ¹H NMR (500 MHz, DMSO-d₆) δ 9.52 (s, 2H), 9.01 (s, 1H), 8.17 (d, J=8.9 Hz, 1H), 7.95 (d, J=8.9 Hz, 1H), 7.78 (s, 1H), 7.27 (s, 1H), 6.65 (s, 2H), 3.50 (t, J=6.4 Hz, 2H), 2.80 (t, J=6.2 Hz, 2H), 2.03-1.98 (m, 2H), 1.83-1.73 (m, 2H), ¹³C NMR (126 MHz, DMSO-d₆) δ 154.17, 150.35, 146.76, 136.24, 133.69, 129.63, 127.03, 122.96, 122.04, 121.27, 119.85, 113.64, 109.26, 109.26, 106.74, 31.31, 28.26, 21.99, 21.58; HRMS (ESI) m/z calcd for C₂₁H₁₉N₂O₃ [M+H]⁺ 347.1395, found 347.1390.

### 4-(6,7,8,9-Tetrahydro-1H-pyrrolo[2,3-c]phenanthridin-5-yl)phenol

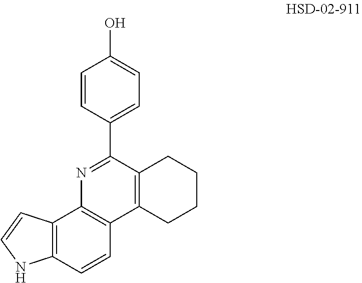 [SMILES: Oc1ccc(-c2nc3c(ccc4[nH]ccc43)c3c2CCCC3)cc1]

Method A: Yellow solid (163 mg, 52%). ¹H NMR (500 MHz, Methanol-d₄) δ 8.05-7.92 (m, 2H), 7.58-7.52 (m, 3H), 7.48 (d, J=3.2 Hz, 1H), 7.07 (d, J=8.3 Hz, 2H), 3.51 (t, J=6.5 Hz, 2H), 2.82 (d, J=6.4 Hz, 2H), 2.06 (dd, J=7.7, 4.3 Hz, 2H), 1.92-1.77 (m, 2H), ¹³C NMR (126 MHz, Methanol-d₄) δ 160.10, 155.63, 152.51, 137.17, 131.84, 130.93, 128.03, 125.79, 122.79, 122.70, 116.47, 116.22, 115.74, 115.44, 101.89, 27.60, 27.36, 21.68, 21.30; HRMS (ESI) m/z calcd for C₂₁H₁₉N₂O [M+H]⁺ 315.1497, found 315.1495.

### 7-(2,2-Difluorobenzo[d][1,3]dioxol-5-yl)-8,9,10,11-tetrahydro-3H-pyrazolo[4,3-a]phenanthridine

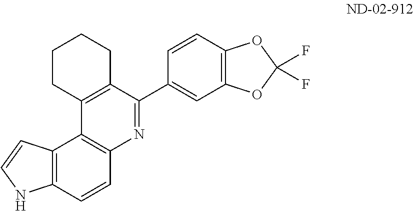 [SMILES: FC1(F)Oc2ccc(-c3nc4ccc5[nH]ccc5c4c4c3CCCC4)cc2O1]

Method A: Off-white solid (57 mg, 15%)¹H NMR (500 MHz, Methanol-d₄) δ 7.78 (dd, J=9.0, 3.6 Hz, 1H), 7.68 (dd, J=9.1, 3.6 Hz, 1H), 7.36 (dt, J=40.7, 3.0 Hz, 2H), 7.32-7.20 (m, 2H), 7.15 (d, J=3.5 Hz, 1H), 3.42 (q, J=5.7 Hz, 2H), 2.69 (q, J=5.4 Hz, 2H), 2.05-1.94 (m, 2H), 1.82-1.69 (m, 2H); ¹³C NMR (126 MHz, MeOD) δ 154.25, 144.03, 143.32, 143.22, 142.52, 137.40, 133.29, 131.73, 127.27, 124.87, 122.96, 122.87, 121.94, 120.19, 116.15, 110.39, 108.94, 105.81, 29.90, 28.49, 22.35, 22.03; HRMS (ESI) m/z calcd for C₂₁H₁₆F₂N₃O₂ [M+H]⁺ 380.1211, found 380.1216.

### 7-(1-Methyl-1H-imidazol-5-yl)-8,9,10,11-tetrahydro-3H-pyrrolo[3,2-a]phenanthridine

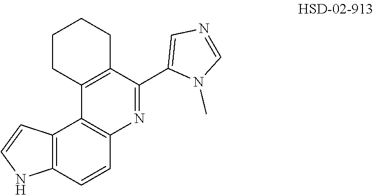 [SMILES: Cn1cncc1-c1nc2ccc3[nH]ccc3c2c2c1CCCC2]

Method A: Off-white solid (151 mg, 50%). ¹H NMR (500 MHz, Methanol-d₄) δ 7.79 (d, J=7.9 Hz, 2H), 7.66 (d, J=8.9 Hz, 1H), 7.40 (d, J=3.1 Hz, 1H), 7.18 (s, 1H), 7.12 (d, J=3.1 Hz, 1H), 3.60 (s, 3H), 3.38 (t, J=6.5 Hz, 2H), 2.75 (t, J=6.3 Hz, 2H), 2.00-1.94 (m, 2H), 1.81 (q, J=6.0, 5.6 Hz, 2H), ¹³C NMR (126 MHz, Methanol-d₄) δ 144.67, 144.01, 142.83, 138.47, 133.43, 130.97, 129.25, 128.07, 122.97, 122.14, 120.11, 116.34, 105.94, 31.45, 29.82, 27.83, 22.28, 21.90; HRMS (ESI) m/z calcd for C₁₉H₁₉N₄ [M+H]⁺ 303.1609, found 303.1599.

### 4-(8,9,10,11-Tetrahydro-3H-pyrrolo[3,2-a]phenanthridin-7-yl)benzene-1,3-diol

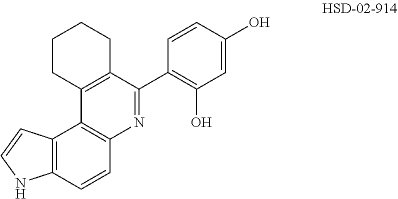 [SMILES: Oc1ccc(-c2nc3ccc4[nH]ccc4c3c3c2CCCC3)c(O)c1]

Method A: Yellow solid (142 mg, 43%). ¹H NMR (500 MHz, Methanol-d₄) δ 7.77 (dd, J=8.9, 0.8 Hz, 1H), 7.68 (d, J=8.9 Hz, 1H), 7.42 (d, J=3.1 Hz, 1H), 7.19 (dd, J=3.2, 0.9 Hz, 1H), 7.09 (d, J=8.9 Hz, 1H), 6.46-6.42 (m, 2H), 3.49 (t, J=6.5 Hz, 2H), 2.80 (t, J=6.3 Hz, 2H), 2.09-2.02 (m, 2H), 1.87-1.78 (m, 2H), ¹³C NMR (126 MHz, Methanol-d₄) δ 158.65, 156.16, 154.02, 143.81, 141.57, 133.18, 130.73, 129.43, 122.78, 122.59, 121.13, 120.30, 118.60, 115.81, 106.34, 105.68, 102.35, 29.99, 27.42, 22.52, 22.05; HRMS (ESI) m/z calcd for C₂₁H₁₉N₂O₂ [M+H]⁺331.1446, found 331.1440.

### 4-(3,8,9,10,11,12-Hexahydrocyclohepta[c]pyrrolo[3,2-f]quinolin-7-yl)phenol

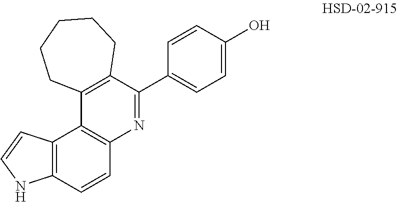 [SMILES: Oc1ccc(-c2nc3ccc4[nH]ccc4c3c3c2CCCCC3)cc1]

Method A: Off white solid (187 mg, 57%). ¹H NMR (500 MHz, DMSO-d₆) δ 11.67 (s, 1H), 9.52 (s, 1H), 7.72 (s, 1H), 7.56 (d, J=8.9 Hz, 1H), 7.45 (d, J=2.8 Hz, 1H), 7.29 (d, J=8.1 Hz, 2H), 7.07 (d, J=3.1 Hz, 1H), 6.85 (s, 2H), 3.55 (dd, J=6.8, 3.3 Hz, 2H), 3.08-2.96 (m, 2H), 1.88 (dq, J=16.5, 4.5 Hz, 4H), 1.63 (p, J=5.1 Hz, 2H), ¹³C NMR (126 MHz, DMSO-d₆) δ 157.25, 157.11, 154.98, 148.20, 144.09, 133.98, 133.26, 133.10, 132.98, 130.91, 124.12, 123.99, 123.82, 121.64, 120.00, 116.78, 116.72, 115.09, 115.00, 105.09, 31.64, 30.68, 30.11, 27.75, 25.12; HRMS (ESI) m/z calcd for C₂₂H₂₁N₂O [M+H]⁺ 329.1653, found 329.1652.

### 4-(8,9,10,11-Tetrahydro-3H-[1,2,3]triazolo[4,5-a]phenanthridin-7-yl)phenol

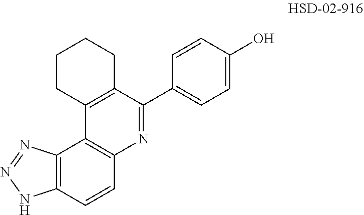 [SMILES: Oc1ccc(-c2nc3ccc4[nH]nnc4c3c3c2CCCC3)cc1]

Method A: Off white solid (237 mg, 75%). ¹H NMR (500 MHz, DMSO-d₆) δ 9.73 (s, 1H), 8.04 (d, J=9.1 Hz, 1H), 7.89 (d, J=9.1 Hz, 1H), 7.44 (d, J=8.3 Hz, 2H), 6.89 (d, J=8.4 Hz, 2H), 3.67 (s, 2H), 2.81 (s, 1H), 2.03-1.88 (m, 2H), 1.82-1.59 (m, 2H); ¹³C NMR (126 MHz, DMSO) δ 158.08, 143.69, 131.01, 130.34, 118.90, 115.25, 30.03, 28.96, 26.90, 24.76. HRMS (ESI) m/z calcd for C₁₉H₁₇N₄O [M+H]⁺317.1402, found 317.1408.

### 4-(2,3,4,8-Tetrahydro-1H-pyrrolo[3,2-b]phenanthridin-5-yl)phenol

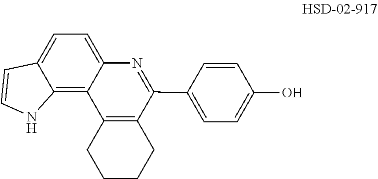 [SMILES: Oc1ccc(-c2nc3ccc4cc[nH]c4c3c3c2CCCC3)cc1]

Method A: Off white solid (185 mg, 59%). ¹H NMR (500 MHz, Methanol-d₄) δ 7.87 (d, J=8.7 Hz, 1H), 7.62 (d, J=8.7 Hz, 1H), 7.43 (d, J=3.1 Hz, 1H), 7.34 (d, J=8.5 Hz, 1H), 6.92 (d, J=8.5 Hz, 2H), 6.70 (d, J=3.1 Hz, 1H), 3.50 (t, J=6.5 Hz, 2H), 2.78 (t, J=6.3 Hz, 2H), 2.13-2.05 (m, 2H), 1.86-1.80 (m, 2H); ¹³C NMR (126 MHz, MeOD) δ 157.34, 156.57, 143.27, 141.29, 131.70, 130.00, 128.49, 127.57, 125.52, 124.19, 123.50, 119.83, 117.04, 114.57, 102.65, 29.32, 28.61, 22.41, 22.04; HRMS (ESI) m/z calcd for C₂₁H₁₉N₂O [M+H]⁺ 315.1497, found 315.1495.

### 4-(2,3,4,8-Tetrahydro-1H-pyrrolo[3,2-b]phenanthridin-5-yl)phenol

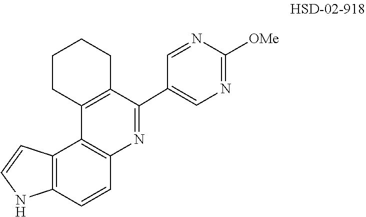 [SMILES: COc1ncc(-c2nc3ccc4[nH]ccc4c3c3c2CCCC3)cn1]

Method A: Off white solid (178 mg, 54%). ¹H NMR (500 MHz, DMSO-d₆) δ 11.81 (s, 1H), 8.85 (s, 2H), 7.81 (d, J=8.8 Hz, 1H), 7.66 (d, J=8.8 Hz, 1H), 7.51 (t, J=2.8 Hz, 1H), 7.13 (t, J=2.5 Hz, 1H), 3.99 (s, 3H), 3.37 (t, J=6.5 Hz, 2H), 2.84 (t, J=6.2 Hz, 2H), 1.99-1.91 (m, 2H), 1.75 (dd, J=7.5, 4.3 Hz, 2H); ¹³C NMR (126 MHz, DMSO) δ 164.77, 159.82, 149.96, 143.68, 142.81, 133.37, 129.18, 127.97, 124.12, 124.09, 122.80, 120.25, 116.84, 106.43, 55.22, 29.96, 28.70, 22.75, 22.49; HRMS (ESI) m/z calcd for C₂₀H₁₉N₄O [M+H]⁺ 331.1558, found 331.1553.

### 2,2′-((4-(8,9,10,11-Tetrahydro-3H-pyrrolo[3,2-a]phenanthridin-7-yl)phenyl)azanediyl)diethanol

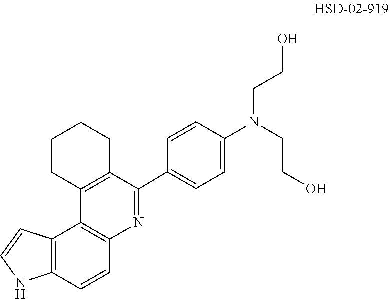 [SMILES: OCCN(CCO)c1ccc(-c2nc3ccc4[nH]ccc4c3c3c2CCCC3)cc1]

Method A: Yellow solid (209 mg, 70%). ¹H NMR (500 MHz, Methanol-d₄) δ 8.00 (d, J=8.9 Hz, 1H), 7.77 (d, J=8.9 Hz, 1H), 7.59 (d, J=3.1 Hz, 1H), 7.48-7.43 (m, 2H), 7.28 (d, J=3.2 Hz, 1H), 6.95 (d, J=8.9 Hz, 2H), 3.79 (t, J=6.0 Hz, 4H), 3.65 (t, J=6.0 Hz, 4H), 2.90 (t, J=6.2 Hz, 2H), 2.12-2.06 (m, 2H), 1.84 (td, J=6.2, 3.2 Hz, 2H); ¹³C NMR (126 MHz, MeOD) δ 152.36, 151.09, 149.41, 136.32, 133.50, 130.36, 129.04, 124.91, 122.68, 121.37, 120.18, 118.91, 115.34, 111.33, 106.08, 58.85, 53.41, 30.85, 28.37, 21.94, 21.66; HRMS (ESI) m/z calcd for C₂₅H₂₈N₃O₂ [M+H]⁺ 402.2181, found 402.2179.

### 2-Fluoro-4-(8,9,10,11-tetrahydro-3H-pyrazolo[4,3-a]phenanthridin-7-yl)phenol

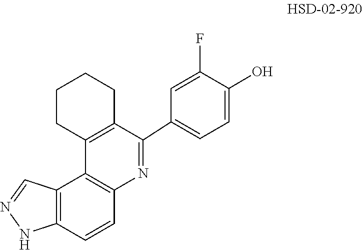 [SMILES: Oc1ccc(-c2nc3ccc4[nH]ncc4c3c3c2CCCC3)cc1F]

Method A: Off white solid (207 mg, 62%). ¹H NMR (500 MHz, DMSO-d₆) δ 10.81 (s, 1H), 8.83 (s, 1H), 8.26 (d, J=9.2 Hz, 1H), 8.21 (d, J=9.2 Hz, 1H), 7.66 (dd, J=11.8, 2.2 Hz, 1H), 7.42 (dd, J=8.4, 2.1 Hz, 1H), 7.26 (t, J=8.6 Hz, 1H), 3.49 (t, J=6.4 Hz, 2H), 2.83 (t, J=6.2 Hz, 2H), 2.07-2.00 (m, 2H), 1.85-1.76 (m, 2H); ¹³C NMR (126 MHz, DMSO) δ 151.89, 149.96, 147.88, 135.32, 132.02, 127.33, 123.16, 120.36, 118.65, 118.49, 118.29, 114.87, 30.92, 28.11, 21.80, 21.69; HRMS (ESI) m/z calcd for C₂₀H₁₇FN₃O [M+H]⁺ 334.1355, found 334.1352.

### 7-(Pyridin-4-yl)-8,9,10,11-tetrahydro-3H-pyrrolo[3,2-a]phenanthridine

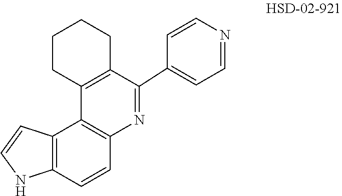 [SMILES: c1cc(-c2nc3ccc4[nH]ccc4c3c3c2CCCC3)ccn1]

Method A: Off white solid (183 mg, 61%). ¹H NMR (500 MHz, DMSO-d₆) δ 11.81 (s, 1H), 8.67 (dt, J=4.4, 1.5 Hz, 2H), 7.81 (d, J=8.8 Hz, 1H), 7.65 (d, J=8.9 Hz, 1H), 7.60-7.55 (m, 2H), 7.54-7.48 (m, 1H), 7.14 (s, 1H), 3.40 (t, J=6.6 Hz, 2H), 2.77 (t, J=6.1 Hz, 2H), 2.01-1.94 (m, 2H), 1.75 (q, J=5.9 Hz, 2H); ¹³C NMR (126 MHz, DMSO) δ 153.34, 149.84, 149.10, 143.44, 142.78, 133.41, 127.19, 124.49, 124.16, 124.08, 122.90, 120.26, 116.87, 106.42, 29.94, 28.67, 22.76, 22.39; HRMS (ESI) m/z calcd for C₂₀H₁₈N₃ [M+H]⁺ 300.1500, found 300.1508.

### 4-(2,3,4,9-Tetrahydro-1H-indolo[3,2-a]phenanthridin-5-yl)phenol

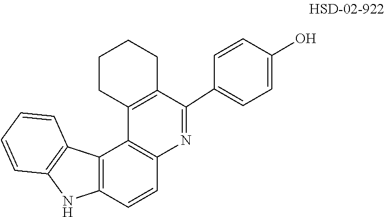 [SMILES: Oc1ccc(-c2nc3ccc4[nH]c5ccccc5c4c3c3c2CCCC3)cc1]

Method A: Brown solid (175 mg, 48%). ¹H NMR (500 MHz, DMSO-d₆) δ 8.52 (d, J=8.5 Hz, 1H), 8.25 (dd, J=8.0, 3.7 Hz, 2H), 7.73 (dd, J=8.5, 4.1 Hz, 1H), 7.58 (ddd, J=8.3, 5.4, 2.6 Hz, 2H), 7.51 (td, J=7.7, 2.5 Hz, 1H), 7.33 (t, J=7.7 Hz, 1H), 7.09-7.00 (m, 2H), 3.86 (t, J=6.0 Hz, 2H), 2.89 (t, J=6.7 Hz, 2H), 1.91 (p, J=6.6 Hz, 2H), 1.81 (q, J=6.3 Hz, 2H); ¹³C NMR (126 MHz, DMSO) δ 159.66, 151.29, 145.82, 140.03, 139.23, 132.51, 131.70, 130.16, 128.83, 125.54, 125.33, 125.13, 123.01, 120.08, 116.30, 115.84, 113.14, 112.73, 110.76, 33.12, 26.84, 21.64, 21.36; HRMS (ESI) m/z calcd for C₂₅H₂₁N₂O [M+H]⁺ 365.1653, found 365.1647.

### 4-(3-Methyl-8,9,10,11-tetrahydro-3H-pyrrolo[3,2-a]phenanthridin-7-yl)phenol

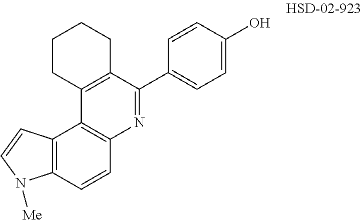 [SMILES: Cn1ccc2c3c4c(c(-c5ccc(O)cc5)nc3ccc21)CCCC4]

Method A: Yellow solid (246 mg, 75%). ¹H NMR (500 MHz, Methanol-d₄) δ 8.23 (d, J=9.0 Hz, 1H), 7.89 (d, J=9.1 Hz, 1H), 7.66 (d, J=3.1 Hz, 1H), 7.54 (d, J=8.5 Hz, 2H), 7.35 (d, J=3.1 Hz, 1H), 7.06 (d, J=8.4 Hz, 2H), 4.08 (s, 3H), 3.65 (t, J=6.4 Hz, 2H), 2.92 (t, J=6.2 Hz, 2H), 2.14 (dq, J=8.6, 5.9, 4.6 Hz, 2H), 1.94-1.85 (n, 2H); ¹³C NMR (126 MHz, MeOD) δ 160.06, 154.70, 150.64, 134.12, 133.70, 130.78, 130.35, 129.85, 123.15, 122.88, 120.71, 118.64, 115.50, 112.77, 105.58, 32.40, 31.23, 27.96, 21.60, 21.28; HRMS (ESI) m/z calcd for C₂₂H₂₁N₂O [M+H]⁺ 329.1653, found 329.1645.

### 5-(8,9,10,11-Tetrahydro-3H-pyrazolo[4,3-a]phenanthridin-7-yl)pyridin-2-ol

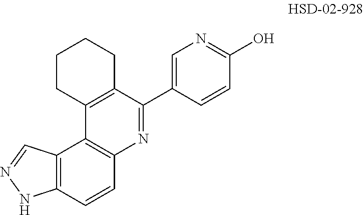 [SMILES: Oc1ccc(-c2nc3ccc4[nH]ncc4c3c3c2CCCC3)cn1]

Method A: Off white solid (158 mg, 50%). H NMR (500 MHz, DMSO-d₆) δ 11.86 (s, 1H), 8.53 (s, 1H), 7.83 (d, J=9.0 Hz, 1H), 7.80-7.73 (m, 2H), 7.65 (d, J=2.6 Hz, 1H), 6.42 (d, J=9.4 Hz, 1H), 3.25 (t, J=6.6 Hz, 2H), 2.84 (t, J=6.1 Hz, 2H), 2.01-1.92 (m, 2H), 1.80-1.68 (m, 2H); ¹³C NMR (126 MHz, DMSO) δ 162.21, 152.68, 143.74, 142.84, 142.59, 136.37, 129.51, 129.43, 121.76, 119.38, 118.73, 116.21, 29.69, 28.78, 22.59, 22.54; HRMS (ESI) m/z calcd for C₁₉H₁₇N₄O [M+H]⁺ 317.1402, found 317.1403.

### 4-(8,9,10,11-Tetrahydro-3H-pyrazolo[4,3-a]phenanthridin-7-yl)aniline

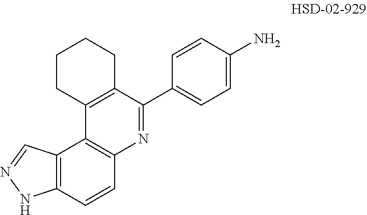 [SMILES: Nc1ccc(-c2nc3ccc4[nH]ncc4c3c3c2CCCC3)cc1]

Method A: Orange solid (192 mg, 61%). ¹H NMR (500 MHz, DMSO-d₆) δ 8.73 (s, 1H), 8.16 (t, J=11.5 Hz, 2H), 7.45 (d, J=8.5 Hz, 2H), 6.79 (d, J=8.3 Hz, 2H), 3.46 (s, 2H), 2.98-2.78 (m, 2H′), 2.09-1.92 (m, 2H), 1.82-1.63 (m, 2H); ¹³C NMR (126 MHz, DMSO) δ 162.75, 154.28, 150.08, 138.83, 136.20, 131.25, 130.62, 129.84, 124.50, 121.76, 115.68, 114.96, 113.80, 111.38, 30.33, 28.84, 22.22; HRMS (ESI) m/z calcd for C₂₁H₁₉N₄ [M+H]⁺ 315.1609, found 315.1619.

### 5-(8,9,10,11-Tetrahydro-3H-pyrrolo[3,2-a]phenanthridin-7-yl)pyridin-2-ol

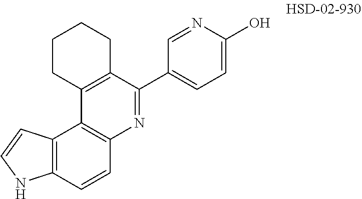 [SMILES: Oc1ccc(-c2nc3ccc4[nH]ccc4c3c3c2CCCC3)cn1]

Method A: Off-white solid (170 mg, 54%). ¹H NMR (500 MHz, DMSO-d₆) δ 10.47 (s, 1H), 8.47 (d, J=2.1 Hz, 1H), 8.36 (q, J=9.2 Hz, 2H), 7.79 (d, J=2.2 Hz, 1H), 7.66-7.56 (m, 2H), 7.10-6.98 (m, 2H), 3.56 (t, J=6.4 Hz, 2H), 2.82 (t, J=6.2 Hz, 2H), 2.00 (dq, J=8.7, 5.8, 4.5 Hz, 2H), 1.85-1.65 (m, 2H H); ¹³C NMR (126 MHz, DMSO) δ 160.63, 153.59, 153.01, 148.23, 132.05, 131.37, 122.86, 121.34, 119.47, 116.00, 110.37, 30.67, 28.32, 21.74, 21.57; HRMS (ESI) m/z calcd for C₂₀H₁₈N₃O [M+H]⁺ 316.1449, found 316.1455.

### 4-(8,9,10,11-Tetrahydrofuro[3,2-a]phenanthridin-7-yl)phenol

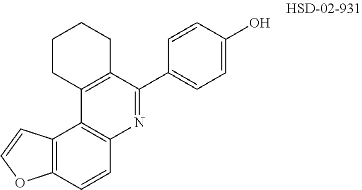 [SMILES: Oc1ccc(-c2nc3ccc4occc4c3c3c2CCCC3)cc1]

Method A: Off-white solid (218 mg, 69%). ¹H NMR (500 MHz, DMSO-d₆) δ 8.47 (d, J=2.1 Hz, 1H), 8.36 (q, J=9.2 Hz, 2H), 7.79 (d, J=2.2 Hz, 1H), 7.61 (d, J=8.6 Hz, 2H), 7.06 (d, J=8.6 Hz, 2H), 3.56 (t, J=6.4 Hz, 2H), 2.82 (t, J=6.2 Hz, 2H), 2.00 (dq, J=8.7, 5.8, 4.5 Hz, 2H), 1.86-1.65 (m, 2H); ¹³C NMR (126 MHz, DMSO) δ 160.63, 153.59, 153.01, 148.23, 132.05, 131.37, 122.86, 121.34, 119.47, 116.00, 110.37, 30.67, 28.32, 21.74, 21.57; HRMS (ESI) m/z calcd for C₂₁H₁₈NO₂ [M+H]⁺ 316.1337, found 316.1343.

### 4-(8,91011-Tetrahydrothieno[3,2-a]phenanthridin-7-yl)phenol

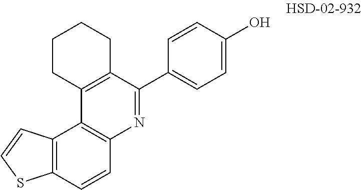 [SMILES: Oc1ccc(-c2nc3ccc4sccc4c3c3c2CCCC3)cc1]

Method A: Off-white solid (245 mg, 74%). ¹H NMR (500 MHz, DMSO-d₆) δ 9.96 (s, 1H), 8.36 (d, J=8.9 Hz, 1H), 8.32 (d, J=5.6 Hz, 1H), 8.08 (d, J=5.5 Hz, 1H), 7.97 (d, J=8.9 Hz, 1H), 7.46 (d, J=8.5 Hz, 2H), 6.94 (d, J=8.5 Hz, 2H), 3.49 (q, J=6.5 Hz, 2H), 2.80 (t, J=6.2 Hz, 2H), 1.94 (dp, J=6.6, 4.6, 2.8 Hz, 2H), 1.79-1.65 (m, 2H); ¹³C NMR (126 MHz, DMSO) δ 158.82, 139.74, 133.74, 131.22, 130.02, 128.68, 127.46, 125.78, 123.88, 115.49, 31.70, 29.13, 22.58, 21.88; HRMS (ESI) m/z calcd for C₂₁H₁₈NOS [M+H]⁺ 332.1109, found 332.1118.

### 2,6-Difluoro-4-(8,9,10,11-tetrahydro-3H-pyrazolo[4,3-a]phenanthridin-7-yl)phenol

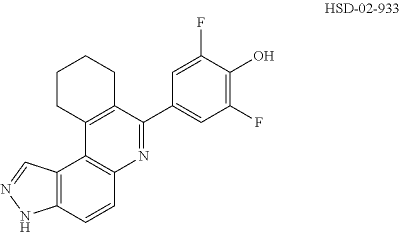 [SMILES: Oc1c(F)cc(-c2nc3ccc4[nH]ncc4c3c3c2CCCC3)cc1F]

Method A: Off-white solid (221 mg, 63%). ¹H NMR (500 MHz, DMSO-d₆) δ 8.76 (s, 1H), 8.22 (S, 2H), 7.66-7.43 (m, 2H), 3.43 (t, J=6.4 Hz, 2H), 2.83 (t, J=6.1 Hz, 2H), 2.19-1.94 (m, 2H), 1.88-1.50 (m, 2H), ¹³C NMR (126 MHz, DMSO) δ 153.27, 151.34, 151.28, 136.31, 131.82, 123.22, 114.73, 114.52, 30.79, 27.97, 21.80, 21.69; HRMS (ESI) m/z calcd for C₂₀H₁₆F₂N₃O [M+H]*352.1261, found 352.1260.

### 4-(8,9,10,11-Tetrahydro-3H-pyrrolo[3,2-a]phenanthridin-7-yl)aniline

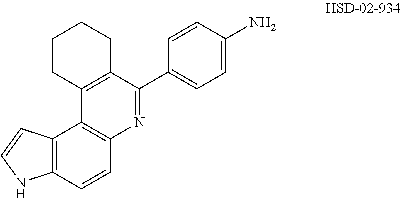 [SMILES: Nc1ccc(-c2nc3ccc4[nH]ccc4c3c3c2CCCC3)cc1]

Method A: Bright yellow solid (194 mg, 62%). ¹H NMR (500 MHz, DMSO-d₆) δ 8.09 (d, J=8.9 Hz, 1H), 7.93 (d, J=8.8 Hz, 1H), 7.72 (s, 1H), 7.40 (d, J=8.2 Hz, 2H), 7.26 (s, 1H), 6.75 (d, J=8.3 Hz, 2H), 5.79 (s, 2H), 3.51 (t, J=6.5 Hz, 2H), 3.32 (s, 2H), 2.85 (t, J=6.2 Hz, 2H), 2.09-1.95 (m, 2H), 1.81-1.68 (m, 2H); ¹³C NMR (126 MHz, DMSO) δ 151.31, 133.58, 133.58, 131.48, 129.37, 129.33, 127.94, 126.34, 122.49, 120.03, 118.73, 113.57, 106.60, 31.13, 28.60, 22.25, 21.94; HRMS (ESI) m/z calcd for C₂₁H₂₀N₃ [M+H]⁺ 314.1657, found 314.1651.

### 2,6-Difluoro-4-(8,9,10,11-tetrahydro-3H-pyrrolo[3,2-a]phenanthridin-7-yl)phenol

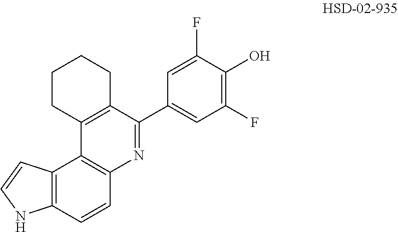 [SMILES: Oc1c(F)cc(-c2nc3ccc4[nH]ccc4c3c3c2CCCC3)cc1F]

Method A: Yellow solid (161 mg, 46%). ¹H NMR (500 MHz, DMSO-d₆) δ 12.43 (s, 1H), 10.91 (s, 1H), 8.13 (d, J=9.0 Hz, 1H), 7.95 (d, J=8.9 Hz, 1H), 7.73 (s, 1H), 7.50 (d, J=7.2 Hz, 2H), 7.28 (s, 1H), 3.52 (t, J=6.5 Hz, 2H), 2.81 (t, J=6.1 Hz, 2H), 2.03-1.99 (m, 2H), 1.78 (dd, J=6.2, 2.7 Hz, 2H); ¹³C NMR (126 MHz, DMSO) δ 153.28, 153.22, 151.35, 151.29, 148.78, 135.89, 133.70, 132.12, 129.36, 126.45, 123.30, 120.58, 119.86, 114.37, 106.78, 31.05, 28.08, 22.12, 21.76; HRMS (ESI) m/z calcd for C₂₁H₁₇F₂N₂O [M+H]⁺ 351.1308, found 351.1302.

### 2-Methyl-4-(8,9,10,11-tetrahydro-3H-pyrrolo[3,2-a]phenanthridin-7-yl)phenol

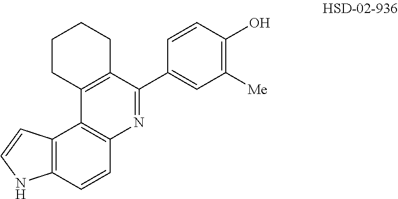 [SMILES: Cc1cc(-c2nc3ccc4[nH]ccc4c3c3c2CCCC3)ccc1O]

Method A: Yellow solid (260 mg, 79%). ¹H NMR (500 MHz, DMSO-d₆) δ 10.31 (s, 1H), 8.19 (d, J=9.0 Hz, 1H), 8.03 (d, J=9.0 Hz, 1H), 7.78 (t, J=2.9 Hz, 1H), 7.47 (t, J=1.5 Hz, 1H), 7.41 (dd, J=8.3, 2.4 Hz, 1H), 7.29 (d, J=2.5 Hz, 1H), 7.10 (d, J=8.3 Hz, 1H), 3.54 (t, J=6.4 Hz, 2H), 2.82 (t, J=6.2 Hz, 2H), 2.06-1.97 (m, 2H), 1.78 (qd, J=6.2, 3.0 Hz, 2H); ¹³C NMR (126 MHz, DMSO) δ 158.45, 154.02, 150.34, 133.74, 132.54, 129.76, 129.19, 127.02, 124.97, 122.99, 122.46, 121.27, 119.85, 115.10, 113.79, 106.76, 31.34, 28.25, 22.01, 21.65, 16.42; HRMS (ESI) m/z calcd for C₂₂H₂₁N₂O [M+H]⁺ 329.1653, found 329.1645.

### 2-Chloro-4-(8,9,10,11-tetrahydro-3H-pyrrolo[3,2-a]phenanthridin-7-yl)phenol

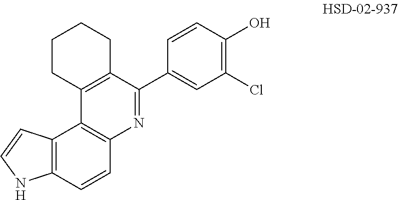 [SMILES: Oc1ccc(-c2nc3ccc4[nH]ccc4c3c3c2CCCC3)cc1Cl]

Method A: Yellow solid (226 mg, 65%). ¹H NMR (500 MHz, DMSO-d₆) δ 11.74 (s, 1H), 7.77 (dd, J=8.9, 0.7 Hz, 1H), 7.62 (d, J=8.8 Hz, 1H), 7.50 (d, J=2.1 Hz, 1H), 7.48 (t, J=2.6 Hz, 1H), 7.34 (dd, 1=8.3.2.1 Hz, 1H), 7.13-7.10 (m, 1H), 7.04 (d, J=8.3 Hz, 1H), 3.37 (d, J=6.6 Hz, 2H), 2.78 (t, J=6.1 Hz, 2H), 1.97 (pd, J=6.1, 2.6 Hz, 2H), 1.77-1.68 (m, 2H); ¹³C NMR (126 MHz, DMSO) δ 154.51, 153.28, 143.38, 142.36, 133.59, 133.20, 130.91, 129.36, 127.54, 124.08, 123.92, 122.39, 120.33, 119.60, 116.47, 116.41, 106.27, 29.99, 29.17, 22.87, 22.60; HRMS (ESI) m/z calcd for C₂₁H₁₈CIN₂O [M+H]⁺ 349.1107, found 349.1112.

### 7-(4-Hydroxyphenyl)-8,9,10,11-tetrahydro-3H-pyrrolo[3,2-a]phenanthridine-9-carbonitrile

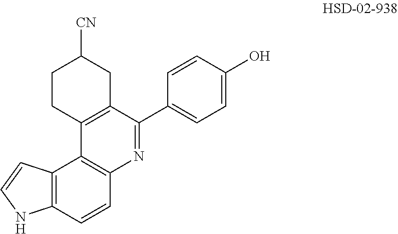 [SMILES: N#CC1CCc2c(c(-c3ccc(O)cc3)nc3ccc4[nH]ccc4c23)C1]

Method A: Yellow solid (190 mg, 56%). ¹H NMR (500 MHz, DMSO-d₆) δ 10.46 (s, 1H), 8.21 (d, J=8.9 Hz, 1H), 8.05 (d, J=9.0 Hz, 1H), 7.81 (s, 1H), 7.62 (d, J=8.6 Hz, 2H), 7.29 (s, 1H), 7.08 (d, J=8.6 Hz, 2H), 3.63 (q, J=5.8 Hz, 2H), 3.40-3.36 (m, 0H), 3.17 (qd, J=16.6, 6.4 Hz, 2H), 2.42-2.35 (m, 1H), 2.32-2.24 (m, 1H); ¹³C NMR (126 MHz, DMSO) δ 160.52, 151.84, 150.21, 134.27, 133.81, 132.07, 127.33, 126.12, 122.49, 122.38, 122.15, 121.66, 119.70, 116.10, 114.00, 106.64, 30.73, 29.32, 24.59, 23.97; HRMS (ESI) m/z calcd for C₂₂H₁₈N₃O [M+H]⁺ 340.1449, found 340.1445.

### 4-(1,2,4,9-Tetrahydropyrano[3,4-c]pyrrolo[3,2-f]quinolin-5-yl)phenol

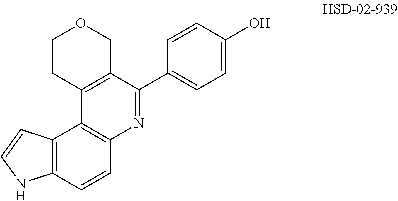 [SMILES: Oc1ccc(-c2nc3ccc4[nH]ccc4c3c3c2COCC3)cc1]

Method A: Yellow solid (193 mg, 61%). ¹H NMR (500 MHz, DMSO-d₆) δ 10.36 (s, 1H), 8.22 (d, J=9.0 Hz, 1H), 8.03 (d, J=9.0 Hz, 1H), 7.81 (t, J=2.9 Hz, 1H), 7.59 (d, J=8.5 Hz, 2H), 7.30 (t, J=2.4 Hz, 1H), 7.03 (d, J=8.5 Hz, 2H), 4.17 (t, J=5.7 Hz, 2H), 3.61 (t, J=5.8 Hz, 2H), 3.42 (s, 2H); ¹³C NMR (126 MHz, DMSO) δ 159.90, 149.19, 133.51, 132.49, 131.49, 130.87, 127.07, 126.51, 122.13, 120.36, 119.93, 116.30, 115.93, 115.62, 105.92, 66.43, 64.15, 29.76; HRMS (ESI) m/z calcd for C₂₀H₁₇N₂O₂ [M+H]⁺ 317.1290, found 317.1290.

### 6-(8,9,10,11-tetrahydro-3H-pyrrolo[3,2-a]phenanthridin-7-yl)pyridin-3-ol

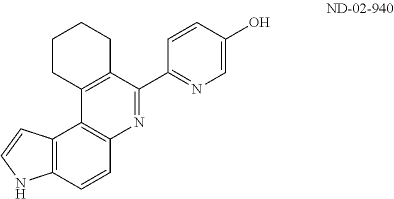 [SMILES: Oc1ccc(-c2nc3ccc4[nH]ccc4c3c3c2CCCC3)nc1]

Method A: Yellow solid (98 mg, 31%). ¹H NMR (500 MHz, DMSO-d₆) δ 11.74 (s, 1H), 10.03 (s, 1H), 8.18 (d, J=2.8 Hz, 1H), 7.77 (d, J=8.9 Hz, 1H), 7.64 (dd, J=12.0, 8.7 Hz, 2H), 7.49 (t, J=2.8 Hz, 1H), 7.29 (dd, J=8.5, 2.9 Hz, 1H), 7.12 (d, J=2.5 Hz, 1H), 3.40-3.35 (m, 2H), 2.95 (t, J=6.3 Hz, 2H), 1.96-1.92 (m, 2H), 1.78-1.69 (m, 2H); ¹³C NMR (126 MHz, DMSO) δ 153.80, 153.32, 151.06, 142.98, 142.40, 136.29, 133.31, 128.21, 125.55, 124.11, 123.92, 123.07, 122.67, 120.36, 116.37, 106.28, 30.10, 28.50, 22.89, 22.51; HRMS (ESI) m/z calcd for C₂₀H₁₈N₃O [M+H]⁺ 316.1450, found 316.1453.

### 4-(8,9,10,11-Tetrahydro-3H-pyrrolo[3,2-a]phenanthridin-7-yl)benzoic acid

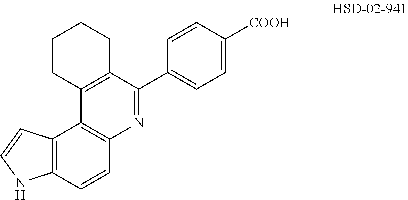 [SMILES: O=C(O)c1ccc(-c2nc3ccc4[nH]ccc4c3c3c2CCCC3)cc1]

Method A: Yellow solid (212 mg, 62%). ¹H NMR (500 MHz, Methanol-d₄) δ8.17 (d, J=8.1 Hz, 2H), 7.87 (d, J=8.9 Hz, 1H), 7.73 (d, J=8.9 Hz, 1H), 7.62 (d, J=8.1 Hz, 2H), 7.49 (d, J=3.1 Hz, 1H), 7.24 (d, J=3.2 Hz, 1H), 3.53 (t, J=6.5 Hz, 2H), 2.76 (t, J=6.2 Hz, 2H), 2.11-2.04 (m, 2H), 1.88-1.79 (m, 2H); ¹³C NMR (126 MHz, MeOD) δ 153.85, 146.36, 142.90, 140.67, 133.41, 132.50, 129.30, 128.80, 127.65, 123.56, 123.12, 120.18, 119.96, 117.15, 105.96, 47.93, 30.20, 28.28, 22.22, 21.85; HRMS (ESI) m/z calcd for C₂₂H₁₉N₂O₂ [M+H]⁺ 343.1446, found 343.1438.

### 5-(8,9,10,11-Tetrahydro-3H-pyrrolo[3,2-a]phenanthridin-7-yl)benzene-1,3-diol

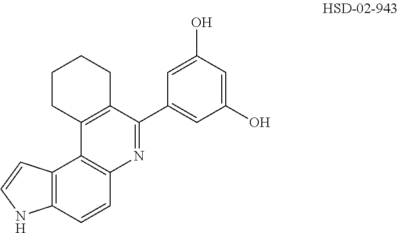 [SMILES: Oc1cc(O)cc(-c2nc3ccc4[nH]ccc4c3c3c2CCCC3)c1]

Method A: Yellow solid (231 mg, 70%). ¹H NMR (500 MHz, DMSO-d₆) δ 9.96 (s, 2H), 8.20 (d, J=8.9 Hz, 1H), 7.96 (d, J=9.0 Hz, 1H), 7.80 (s, 1H), 7.31 (d, J=2.4 Hz, 1H), 6.59-6.47 (m, 3H), 3.54 (t, J=6.5 Hz, 2H), 2.77 (t, J=6.2 Hz, 2H), 2.05-1.97 (m, 2H), 1.82-1.77 (m, 2H), ¹³C NMR (126 MHz, DMSO) δ 159.29, 154.49, 149.99, 133.76, 129.45, 127.13, 123.34, 121.54, 119.85, 113.69, 107.84, 106.82, 104.94, 40.48, 40.31, 40.14, 39.98, 39.81, 39.64, 39.48, 31.31, 27.91, 21.94, 21.44; HRMS (ESI) m/z calcd for C₂₁H₁₉N₂O₂ [M+H]⁺ 331.1446, found 331.1450.

### 5-(8,9,10,11-Tetrahydro-3H-pyrazolo[4,3-a]phenanthridin-7-yl)pyridin-2-amine

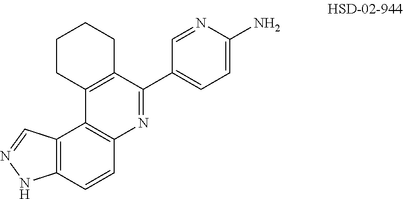 [SMILES: Nc1ccc(-c2nc3ccc4[nH]ncc4c3c3c2CCCC3)cn1]

Method A: Yellow solid (205 mg, 65%). ¹H NMR (500 MHz, Methanol-d₄) δ 8.51 (s, 1H), 8.16 (d, J=2.1 Hz, 1H), 8.14 (dd, J=9.0, 2.2 Hz, 1H), 7.88 (d, J=9.1 Hz, 1H), 7.83 (d, J=9.2 Hz, 1H), 7.09 (d, J=9.0 Hz, 1H), 3.26 (t, J=6.6 Hz, 2H), 2.87 (t, J=6.1 Hz, 2H), 2.06 (ddt, J=9.2, 6.5, 3.2 Hz, 2H), 1.86 (dp, J=9.3, 3.1 Hz, 2H); ¹³C NMR (126 MHz, MeOD) δ 155.32, 150.42, 145.84, 143.67, 143.48, 141.93, 138.13, 130.19, 126.90, 123.86, 122.70, 119.27, 115.60, 111.95, 29.80, 28.11, 21.86, 21.86; HRMS (ESI) m/z calcd for C₁₉H₁₈N₅ [M+H]+ 316.1562, found 316.1555.

### 7-Pyrimidin-5-yl)-8,9,10,11-tetrahydro-3H-pyrazolo[4,3-a]phenanthridine

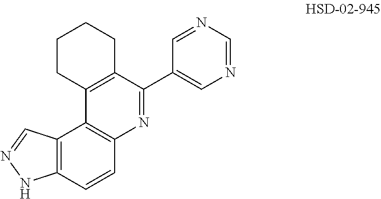 [SMILES: c1ncc(-c2nc3ccc4[nH]ncc4c3c3c2CCCC3)cn1]

Method A: Yellow solid (144 mg, 48%). ¹H NMR (500 MHz, DMSO-d₆) δ ¹H NMR (500 MHz, DMSO-d₆) δ 9.26 (s, 1H), 9.07 (s, 2H), 8.55 (s, 1H), 7.89 (d, J=9.0 Hz, 1H), 7.84 (d, J=11.9 Hz, 2H), 3.31 (s, 4H), 2.84 (t, J=6.1 Hz, 2H), 2.03-1.94 (m, 2H), 1.80-1.71 (m, 2H); ¹³C NMR (126 MHz, DMSO) δ 158.00, 157.25, 150.89, 143.91, 142.97, 138.83, 136.49, 134.77, 129.84, 129.50, 122.42, 116.25, 114.94, 29.62, 28.44, 22.43, 22.38; HRMS (ESI) m/z calcd for C₁₉H₁₇N₄ [M+H]⁺ 301.1453, found 301.1457.

### 5-(8,9,10,11-Tetrahydro-3H-pyrazolo[4,3-a]phenanthridin-7-yl)thiazol-2-amine

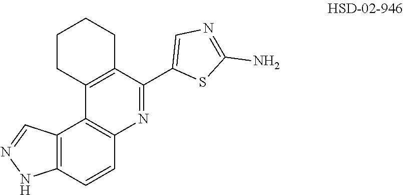 [SMILES: Nc1ncc(-c2nc3ccc4[nH]ncc4c3c3c2CCCC3)s1]

Method A: Yellow solid (166 mg, 52%). ¹H NMR (500 MHz, DMSO-d₆) δ 8.50 (s, 1H), 7.79 (d, J=9.1 Hz, 1H), 7.66 (d, J=9.4 Hz, 1H), 7.51 (s, 1H), 7.23 (s, 2H), 3.28-3.22 (m, 2H), 2.96 (t, J=6.2 Hz, 2H), 1.99-1.93 (m, 2H), 1.90-1.82 (m, 2H); ¹³C NMR (126 MHz, DMSO) δ 170.46, 148.47, 143.26, 142.39, 140.03, 138.39, 136.08, 129.21, 128.99, 128.03, 120.39, 116.44, 114.43, 29.95, 28.90, 22.52, 22.24; HRMS (ESI) m/z calcd for C₁₇H₁₆N₅S [M+H]⁺ 322.1126, found 322.1127.

### 2-Fluoro-4-(8,9,10,11-tetrahydro-3H-pyrrolo[3,2-a]phenanthridin-7-yl)phenol

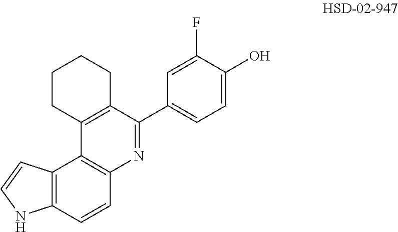 [SMILES: Oc1ccc(-c2nc3ccc4[nH]ccc4c3c3c2CCCC3)cc1F]

Method A: Yellow solid (196 mg, 59%). ¹H NMR (500 MHz, DMSO-d₆) δ 11.75 (s, 2H), 7.85-7.71 (m, 2H), 7.67-7.56 (m, 2H), 7.48 (t, J=2.8 Hz, 1H), 7.10 (t, J=2.4 Hz, 1H), 6.40 (d, J=9.4 Hz, 1H), 3.35 (t, J=6.6 Hz, 2H), 2.84 (t, J=6.1 Hz, 2H), 1.97 (td, J=8.6, 7.2, 4.5 Hz, 2H), 1.75 (tq, J=8.5, 5.4, 3.9 Hz, 2H), ¹³C NMR (126 MHz, DMSO) δ 162.16, 151.74, 143.46, 142.97, 142.51, 136.05, 133.19, 127.71, 123.97, 122.39, 120.28, 119.30, 116.56, 106.27, 30.00, 28.90, 22.83, 22.63; HRMS (ESI) m/z calcd for C₂₁H₁₈FN₂O [M+H]⁺ 333.1405, found 333.1403.

### 5-(8,9,10,11-Tetrahydro-3H-pyrrolo[3,2-a]phenanthridin-7-yl)thiazol-2-amine

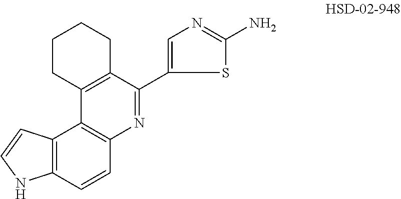 [SMILES: Nc1ncc(-c2nc3ccc4[nH]ccc4c3c3c2CCCC3)s1]

Method A: Yellow solid (112 mg, 35%). ¹H NMR (500 MHz, DMSO-d₆) δ 12.21 (s, 1H) 8.12 (d, J=9.0 Hz, 1H), 8.04-7.94 (m, 1H) 7.56-7.50 (m, 2H), 6.96 (s, 1H), 5.98 (t, J=2.4 Hz, 1H), 3.29 (t, J=6.4 Hz, 2H), 2.84 (s, 2H), 1.96-1.88 (m, 2H), 1.83 (q, J=6.0, 5.3 Hz, 2H); HRMS (ESI) m/z calcd for C₁₈H₁₇N₄S [M+H]⁺ 321.1174, found 321.1177.

### 2-Hydroxy-5-(8,9,10,11-tetrahydro-3H-pyrazolo[4,3-a]phenanthridin-7-yl)benzoic acid

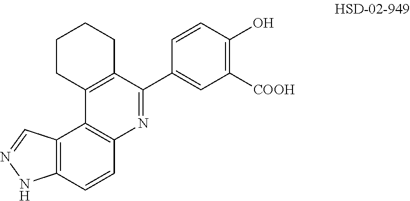 [SMILES: O=C(O)c1cc(-c2nc3ccc4[nH]ncc4c3c3c2CCCC3)ccc1O]

Method A: Yellow solid (187 mg, 52%). ¹H NMR (500 MHz, DMSO-d₆) δ 10.10 (s, 1H), 8.67 (s, 1H), 8.09 (d, J=9.8 Hz, 4H), 3.38-3.31 (m, 2H), 2.78 (t, J=6.2 Hz, 2H), 2.06-1.93 (m, 2H), 1.83-1.72 (m, 2H); HRMS (ESI) m/z calcd for C₂₁H₁₈N₃O₃ [M+H]⁺ 360.1348, found 360.1351.

### Methyl 2-hydroxy-5-(8,9,10,11-tetrahydro-3H-pyrazolo[4,3-a]phenanthridin-7-yl)benzoate

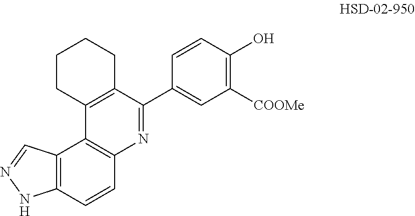 [SMILES: COC(=O)c1cc(-c2nc3ccc4[nH]ncc4c3c3c2CCCC3)ccc1O]

Method A: White solid (208 mg, 56%). ¹H NMR (500 MHz, DMSO-d₆) δ 10.93 (s, 1H), 8.73 (s, 1H), 8.20-8.12 (m, 2H), 8.09 (d, J=2.3 Hz, 1H), 7.87 (dd, J=8.6, 2.4 Hz, 1H), 7.24 (d, J=8.6 Hz, 1H), 3.89 (s, 3H), 3.40 (td, J=6.9, 4.0 Hz, 2H), 2.78 (t, J=6.1 Hz, 2H), 2.06-1.91 (m, 2H), 1.77 (tt, J=8.9, 5.5 Hz, 2H), ¹³C NMR (126 MHz, DMSO) δ 168.54, 168.54, 168.32, 161.15, 151.10, 150.72, 136.76, 132.49, 132.29, 131.52, 124.82, 122.96, 121.86, 118.10, 117.86, 115.02, 114.60, 53.03, 30.66, 28.23, 21.85, 21.79; HRMS (ESI) m/z calcd for C₂₂H₂₀N₃O₃ [M+H]⁺ 374.1505, found 374.1508.

### 3-(8,9,10,11-Tetrahydro-3H-pyrrolo[3,2-a]phenanthridin-7-yl)pyridin-2(1H)-one

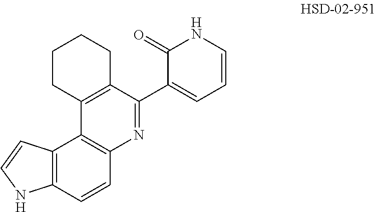 [SMILES: O=c1[nH]cccc1-c1nc2ccc3[nH]ccc3c2c2c1CCCC2]

Method A: Yellow solid (204 mg, 65%). Yellow solid (217 mg, 70%). ¹H NMR (500 MHz, DMSO-d₆) δ 11.76 (s, 1H), 7.80-7.72 (m, 2H), 7.64-7.58 (m, 2H), 7.48 (t, J=2.7 Hz, 1H), 7.10 (t, J=2.3 Hz, 1H), 6.41 (d, J=9.4 Hz, 1H), 3.36 (d, J=6.6 Hz, 2H), 2.84 (t, J=6.2 Hz, 2H), 2.02-1.94 (m, 2H), 1.75 (dp. J=9.3, 3.4, 2.9 Hz, 2H); ¹³C NMR (126 MHz, DMSO) δ 162.19, 151.74, 143.47, 142.98, 142.52, 136.07, 133.20, 127.72, 123.98, 122.40, 120.30, 119.29, 116.56, 106.28, 30.01, 28.91, 22.83, 22.63; HRMS (ESI) m/z calcd for C₂₀H₁₈N₃O [M+H]⁺ 316.1450, found 316.1459.

### Methyl 2-hydroxy-5-(8,9,10,11-tetrahydro-3H-pyrrolo[3,2-a]phenanthridin-7-yl)benzoate

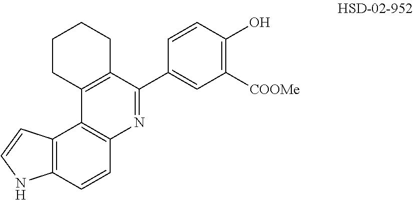 [SMILES: COC(=O)c1cc(-c2nc3ccc4[nH]ccc4c3c3c2CCCC3)ccc1O]

Method A: White solid (189 mg, 51%). ¹H NMR (500 MHz, Methanol-d₄) δ 8.01 (d, J=2.3 Hz, 1H), 7.79 (d, J=8.9 Hz, 1H), 7.71 (d, J=8.8 Hz, 1H), 7.66 (dt, J=8.5, 2.1 Hz, 1H), 7.43 (d, J=3.1 Hz, 1H), 7.20 (d, J=3.2 Hz, 1H), 7.09 (dd, J=8.3, 2.4 Hz, 1H), 3.96 (s, 3H), 3.49 (t, J=6.5 Hz, 2H), 2.77 (t, J=6.2 Hz, 2H), 2.11-2.03 (m, 2H), 1.83 (dp. J=9.4, 3.0 Hz, 2H); ¹³C NMR (126 MHz, MeOD) δ 170.24, 161.08, 154.60, 143.93, 142.64, 136.22, 133.26, 132.11, 130.33, 127.42, 122.82, 121.91, 120.24, 116.86, 116.03, 111.93, 105.77, 51.58, 48.10, 29.95, 28.67, 22.45, 22.16; HRMS (ESI) m/z calcd for C₂₃H₂₁N₂O₃ [M+H]⁺ 373.1552, found 373.1557.

### 2-(7-(4-Hydroxyphenyl)-8,9,10,11-tetrahydro-3H-pyrrolo[3,2-a]phenanthridin-9-yl)isoindoline-1,3-dione 12.11

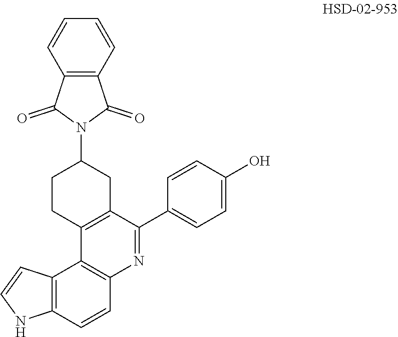 [SMILES: O=C1c2ccccc2C(=O)N1C1CCc2c(c(-c3ccc(O)cc3)nc3ccc4[nH]ccc4c23)C1]

Method A: Yellow solid (197 mg, 43%). ¹H NMR (500 MHz, DMSO-d₆) δ 11.85 (s, 1H), 7.81 (tt, J=4.4, 1.9 Hz, 5H), 7.64 (d, J=8.9 Hz, 1H), 7.50 (d, J=3.1 Hz, 1H), 7.37-7.29 (m, 2H), 7.12 (d, J=3.3 Hz, 1H), 6.85-6.78 (m, 2H), 4.44-4.33 (m, 1H), 3.78-3.69 (m, 1H), 3.69-3.57 (m, 1H), 3.54-3.44 (m, 1H), 2.89-2.81 (m, 1H), 2.79-2.68 (m, 1H), 2.34-2.26 (m, 1H); ¹³C NMR (126 MHz, DMSO) δ 168.40, 157.71, 155.84, 143.60, 141.02, 134.77, 133.23, 132.00, 130.64, 125.66, 124.08, 123.99, 123.38, 121.75, 120.41, 116.72, 115.24, 106.26, 47.58, 32.31, 30.31, 26.40; HRMS (ESI) m/z calcd for C₂₉H₂₂N₃O₃ [M+H]⁺ 460.1661, found 460.1665.

### Benzyl (7-(4-hydroxyphenyl)-8,9,10,11-tetrahydro-3H-pyrrolo[3,2-a]phenanthridin-9-yl)carbamate

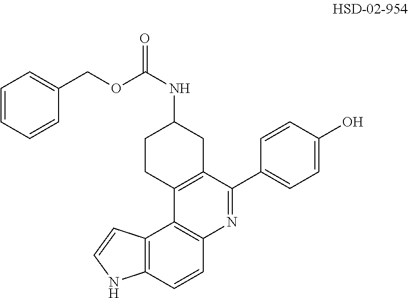 [SMILES: O=C(NC1CCc2c(c(-c3ccc(O)cc3)nc3ccc4[nH]ccc4c23)C1)OCc1ccccc1]

Method A: Yellow solid (240 mg, 52%). ¹H NMR (500 MHz, DMSO-d₆) δ 10.46 (s, 1H), 8.20 (d, J=9.0 Hz, 1H), 8.07 (d, J=8.9 Hz, 1H), 7.78 (d, J=3.0 Hz, 1H), 7.55 (d, J=8.4 Hz, 2H), 7.37-7.19 (m, 6H), 7.08 (s, 11H), 4.97 (d, J=3.2 Hz, 2H), 3.84 (t, J=7.4 Hz, 1H), 3.69 (dt, J=19.6, 5.6 Hz, 1H), 3.56 (dt, J=19.2, 7.4 Hz, 1H), 3.01 (dd, J=16.9, 4.6 Hz, 1H), 2.81 (dd, J=16.6, 8.4 Hz, 1H), 2.31-2.18 (m, 1H), 2.06-1.95 (m, 1H), ¹³C NMR (126 MHz, DMSO) δ 160.39, 156.05, 153.02, 150.33, 137.46, 133.91, 133.75, 131.95, 128.79, 128.27, 127.75, 127.12, 122.61, 122.34, 121.41, 119.89, 116.02, 113.72, 106.77, 65.77, 45.47, 33.92, 29.80, 27.29; HRMS (ESI) m/z calcd for C₂₉H₂₆N₃O₃[M+H]⁺ 4.64.1974 found 464.1970.

### 7-(4-Hydroxyphenyl)-8,9,10,11-tetrahydro-3H-pyrrolo[3,2-a]phenanthridin-9-ol

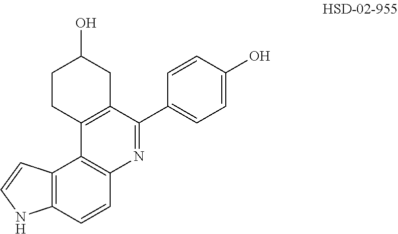 [SMILES: Oc1ccc(-c2nc3ccc4[nH]ccc4c3c3c2CC(O)CC3)cc1]

Method A: Yellow solid (172 mg, 52%). ¹H NMR (500 MHz, DMSO-d) δ 10.43 (s, 1H), 8.19 (d, J=8.9 Hz, 1H), 8.04 (d, J=8.9 Hz, 1H), 7.78 (t, J=2.9 Hz, 1H), 7.65-7.51 (m, 2H), 7.29 (t, J=2.4 Hz, 1H), 7.12-7.00 (m, 2H), 4.05 (tt, J=7.0, 3.1 Hz, 1H), 3.70-3.60 (m, 1H), 3.52 (dt, J=19.2, 6.8 Hz, 1H), 2.99 (dd, J=16.6, 4.1 Hz, 1H), 2.75 (dd, J=16.6, 6.7 Hz, 1H), 2.19-2.09 (m, 11H), 1.99 (dq, J=13.9, 7.0 Hz, 1H), ¹³C NMR (126 MHz, DMSO) δ 160.37, 153.53, 150.30, 133.89, 133.72, 132.01, 127.94, 127.08, 122.53, 121.38, 119.84, 115.99, 113.73, 106.74, 63.25, 36.67, 29.46, 29.03; HRMS (ESI) m/z, calcd for C₂₁H₁₉N₂O₂ [M+H]⁺331.1447, found 331.1452.

### 4-(8,9,10,11-Tetrahydro-3H-pyrrolo[3,2-a]phenanthridin-7-yl)-2-(trifluoromethoxy)phenol

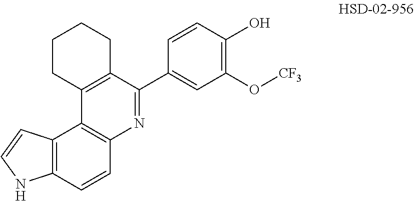 [SMILES: Oc1ccc(-c2nc3ccc4[nH]ccc4c3c3c2CCCC3)cc1OC(F)(F)F]

Method A: off-white solid (242 mg, 61%). ¹H NMR (500 MHz, DMSO-δ 11.16 (s, 1H), 8.22 (d, J=8.9 Hz, 1H), 8.01 (d, J=9.0 Hz, 1H), 7.86-7.75 (m, 2H), 7.60 (dd, J=8.4, 2.2 Hz, 1H), 7.41-7.29 (m, 2H), 1.83-1.76 (m, 2H), ¹³C NMR (126 MHz, DMSO) δ 152.73, 148.65, 136.08, 134.20, 133.76, 130.85, 129.77, 126.94, 125.54, 123.93 (q, J=257 Hz), 123.31, 121.29, 119.80, 118.25, 114.31, 106.81, 31.29, 28.01, 21.96, 21.63; HRMS (ESI) m/z calcd for C₂₂H₁₈F₃N₂O [M+H]⁺399.1320, found 399.1321.

### 3-(8,9,10,11-Tetrahydro-3H-pyrrolo[3,2-a]phenanthridin-7-yl)phenol

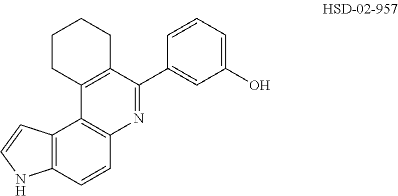 [SMILES: Oc1cccc(-c2nc3ccc4[nH]ccc4c3c3c2CCCC3)c1]

Method A: Yellow solid (188 mg, 60%). ¹H NMR (500 MHz, DMSO-d₆) δ 10.28 (s, 1H), 8.17 (d, J=8.9 Hz, 1H), 7.97 (d, J=8.9 Hz, 1H), 7.77 (t, J=2.9 Hz, 1H), 7.43 (t, J=7.9 Hz, 1H), 7.27-7.16 (m, 2H), 7.09 (td, J=5.2, 2.2 Hz, 2H), 3.45 (t, J=6.5 Hz, 2H), 2.73 (t, J=6.2 Hz, 2H), 2.03-1.95 (m, 2H), 1.81-1.72 (m, 2H), ¹³C NMR (126 MHz, DMSO) δ 158.18, 154.01, 149.81, 133.69, 130.40, 129.35, 126.95, 123.26, 121.30, 120.36, 119.72, 118.04, 116.76, 113.98, 106.74, 31.22, 27.99, 21.92, 21.47; HRMS (ESI) m/z calcd for C₂₁H₁₉N₂O [M+H]⁺ 315.1497, found 315.1499.

### 5-(8,9,10,11-tetrahydro-3H-pyrrolo[3,2-a]phenanthridin-7-yl)pyrimidin-2-amine

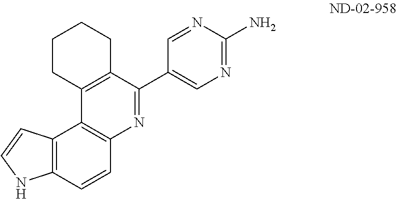 [SMILES: Nc1ncc(-c2nc3ccc4[nH]ccc4c3c3c2CCCC3)cn1]

Method A: Pale yellow solid (185 mg, 59%). ¹H NMR (500 MHz, Methanol-d₄) δ 8.07 (dd, J=2.4, 0.9 Hz, 1H), 7.77 (dd, J=8.9, 0.9 Hz, 1H), 7.69 (d, J=8.9 Hz, 1H), 7.63 (dd, J=8.5, 2.4 Hz, 1H), 7.40 (d, J=3.1 Hz, 1H), 7.14 (dd, J=3.2, 0.9 Hz, 1H), 6.70 (dd, J=8.5, 0.8 Hz, 1H), 3.42 (t, J=6.4 Hz, 2H), 2.78 (t, J=6.2 Hz, 2H), 2.05-1.96 (m, 2H), 1.83-1.75 (m, 2H); ¹³C NMR (126 MHz, DMSO) δ 163.22, 158.63, 158.15, 152.27, 144.04, 142.51, 134.14, 129.66, 129.58, 123.56, 123.39, 121.80, 116.22, 29.69, 28.81, 22.63, 22.56; HRMS (ESI) m/z calcd for C₁₉H₁₈N₅ [M+H]⁺ 316.1562, found 316.1565.

### 2-((Dimethylamino)methyl)-4-(8,9,10,11-tetrahydro-3H-pyrrolo[3,2-a]phenanthridin-7-yl)phenol

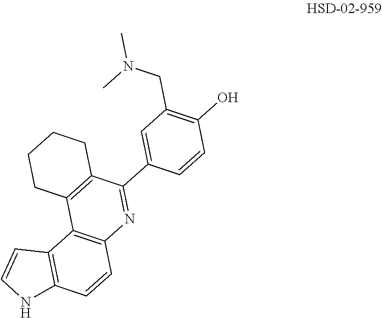 [SMILES: CN(C)Cc1cc(-c2nc3ccc4[nH]ccc4c3c3c2CCCC3)ccc1O]

Method A: Pale yellow solid (170 mg, 46%). ¹H NMR (500 MHz, Methanol-d₄) δ 7.77 (d, J=8.9 Hz, 1H), 7.69 (d, J=9.0 Hz, 1H), 7.41 (s, 1H), 7.33 (dd, J=8.2, 2.2 Hz, 1H), 7.26 (d, J=2.2 Hz, 1H), 7.19-7.15 (m, 1H), 6.92 (d, J=8.2 Hz, 1H), 3.88 (s, 2H), 3.46 (t, J=6.5 Hz, 2H), 2.75 (t, J=6.2 Hz, 2H), 2.05-2.01 (m, 2H), 1.82-1.76 (m, 2H); HRMS (ESI) n/z calcd for C₂₄H₂₆N₃O [M+H]⁺ 372.2076, found 372.2082.

### 2,6-Diiodo-4-(8,9,10,11-tetrahydro-3H-pyrazolo[4,3-a]phenanthridin-7-yl)phenol

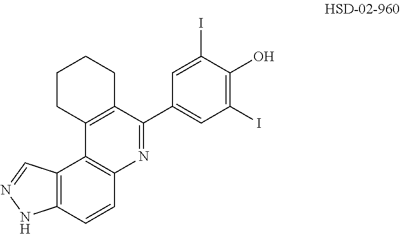 [SMILES: Oc1c(I)cc(-c2nc3ccc4[nH]ncc4c3c3c2CCCC3)cc1I]

Method A: Yellow solid (272 mg, 48%). ¹H NMR (500 MHz, DMSO-d₆) δ 9.79 (s, 1H), 8.57 (s, 1H), 7.94 (s, 2H), 7.92-7.78 (m, 3H), 3.29 (s, 2H), 2.03-1.95 (m, 2H), 1.78-1.70 (m, 2H); HRMS (ESI) m/z calcd for C₂₀H₁₆I₂N₃O [M+H]⁺ 567.9383, found 567.9388.

### 3-(8,9,10,11-Tetrahydro-3H-pyrazolo[4,3-a]phenanthridin-7-yl)phenol

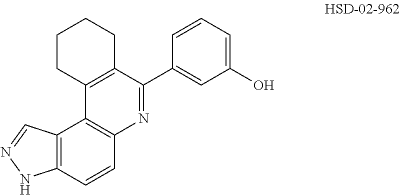 [SMILES: Oc1cccc(-c2nc3ccc4[nH]ncc4c3c3c2CCCC3)c1]

Method A: White solid (195 mg, 62%). ¹H NMR (500 MHz, DMSO-d₆) δ 10.18 (s, 1H), 8.72 (s, 1H), 8.13 (dd, J=37.4, 8.8 Hz, 2H), 7.42 (t, J=7.9 Hz, 1H), 7.17-7.03 (m, 3H), 3.37 (q, J=8.2, 6.3 Hz, 2H), 2.75 (t, J=6.2 Hz, 2H), 2.00 (p, J=6.5, 6.1 Hz, 2H), 1.76 (dd, J=11.3, 5.9 Hz, 2H), ¹³C NMR (126 MHz, DMSO) δ 158.08, 152.26, 151.41, 135.68, 134.33, 131.45, 130.29, 123.05, 120.90, 120.40, 117.83, 116.79, 114.86, 30.73, 28.07, 21.77, 21.59; HRMS (ESI) m/z calcd for C₁₉H₁₈N₅ [M+H]⁺ 316.1562, found 316.1555.

### 3-(8,9,10,11-Tetrahydro-3H-pyrazolo[4,3-a]phenanthridin-7-yl)pyridin-2(1H)-one

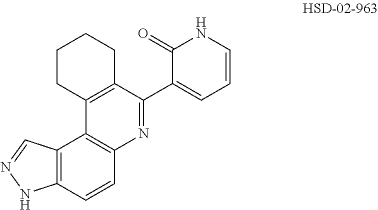 [SMILES: O=c1[nH]cccc1-c1nc2ccc3[nH]ncc3c2c2c1CCCC2]

Method A: Off-white solid (214 mg, 67%). ¹H NMR (500 MHz, DMSO-d₆) δ 8.53 (s, 1H), 7.83 (d, J=9.0 Hz, 1H), 7.81-7.73 (m, 2H), 7.65 (d, J=2.7 Hz, 1H), 6.42 (d, J=9.4 Hz, 1H), 3.25 (t, J=6.5 Hz, 2H), 2.84 (t, J=6.0 Hz, 2H), 1.97 (dq, J=8.4, 5.7 Hz, 2H), 1.78-1.69 (m, 2H); ¹³C NMR (126 MHz, DMSO) δ 162.24, 152.74, 143.82, 142.86, 142.53, 140.86, 139.70, 136.38, 134.04, 129.50, 121.76, 119.36, 118.81, 116.19, 115.37, 29.69, 28.79, 22.61, 22.55; HRMS (ESI) m/z calcd for C₁₉H₁₇N₄O [M+H]⁺ 317.1402, found 317.1411.

### Benzyl (7-(4-hydroxyphenyl)-8,9,10,11-tetrahydro-3H-pyrazolo[4,3-a]phenanthridin-9-yl)carbamate

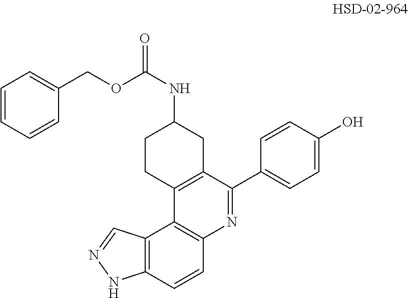 [SMILES: O=C(NC1CCc2c(c(-c3ccc(O)cc3)nc3ccc4[nH]ncc4c23)C1)OCc1ccccc1]

Method A: Off-white solid (241 mg, 52%). ¹H NMR (500 MHz, Methanol-d₄) δ 8.66 (d, J=9.1 Hz, 1H), 8.08 (dd, J=9.2, 6.3 Hz, 1H), 7.93 (dd, J=9.3, 5.3 Hz, 1H), 7.50 (d, J=8.2 Hz, 2H), 7.29-7.20 (m, 5H), 7.02 (d, J=8.2 Hz, 2H), 5.01 (s, 2H), 3.97-3.84 (m, 1H), 3.67-3.56 (m, 1H), 3.54-3.41 (m, 1H), 3.18-3.08 (m, 1H), 2.95-2.82 (m, 1H), 2.42-2.29 (m, 1H), 2.11-2.01 (m, 1H), ¹³C NMR (126 MHz, MeOD) δ 161.72, 159.87, 156.89, 152.70, 150.63, 139.58, 136.84, 136.35, 130.82, 129.46, 128.03, 127.56, 127.34, 123.73, 122.33, 120.83, 119.33, 118.00, 115.47, 114.97, 66.04, 48.13, 48.08, 47.96, 47.79, 47.62, 47.45, 47.28, 47.11, 45.66, 33.71, 29.28, 26.87; HRMS (ESI) m/z calcd for C₂₈H₂₅N₄O₃ [M+H]⁺ 465.1927, found 465.1927.

### 7-(4-Hydroxyphenyl)-8,9,10,11-tetrahydro-3H-pyrazolo[4,3-a]phenanthridin-9-ol

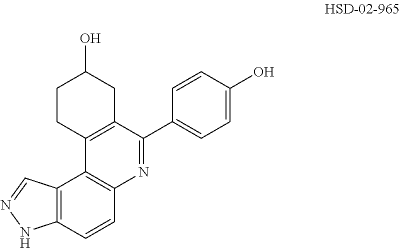 [SMILES: Oc1ccc(-c2nc3ccc4[nH]ncc4c3c3c2CC(O)CC3)cc1]

Method A: Off-white solid (211 mg, 64%). ¹H NMR (500 MHz, DMSO-d₆) δ 9.87 (s, 1H), 8.57 (s, 1H), 7.97-7.80 (m, 2H), 7.45 (d, J=8.1 Hz, 2H), 6.93 (d, J=8.1 Hz, 2H), 4.92 (s, 1H), 3.95 (tt, J=7.8, 3.3 Hz, 1H), 3.41 (dt, J=20.7, 4.7 Hz, 1H), 3.27 (p, J=7.1 Hz, 1H), 2.96 (dd, J=16.6, 4.2 Hz, 1H), 2.74 (dd, J=16.5, 7.4 Hz, 1H), 2.13 (dt, J=13.1, 6.2 Hz, 1H), 1.90 (dq, J=15.0, 7.8 Hz, 1H), ¹³C NMR (126 MHz, DMSO) δ 158.44, 155.40, 144.50, 141.23, 138.96, 136.06, 131.24, 129.27, 128.15, 126.83, 121.32, 115.90, 115.38, 64.42, 37.63, 30.18, 28.16; HRMS (ESI) m/z calcd for C₂₀H₁₈N₃O₂ [M+H]⁺ 332.1399, found 332.1402.

### 2-(7-(4-Hydroxyphenyl)-8,9,10,11-tetrahydro-3H-pyrazolo[4,3-a]phenanthridin-9-yl)isoindoline-1,3-dione

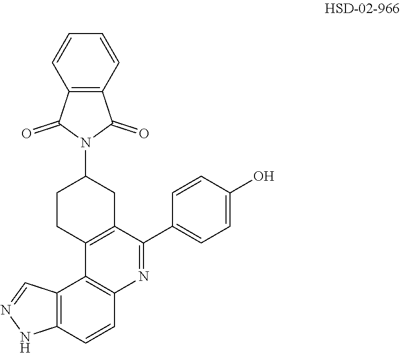 [SMILES: O=C1c2ccccc2C(=O)N1C1CCc2c(c(-c3ccc(O)cc3)nc3ccc4[nH]ncc4c23)C1]

Method A: Off-white solid (239 mg, 52%). ¹H NMR (500 MHz, DMSO-d₆) δ 9.56 (s, 1H), 8.57 (s, 1H), 7.89-7.84 (m, 2H), 7.83-7.78 (m, 4H), 7.36 (d, J=8.6 Hz, 1H), 6.82 (d, J=8.6 Hz, 1H), 4.45-4.33 (m, 1H), 3.73-3.59 (m, 2H), 3.53-3.39 (m, 1H), 2.92-2.85 (m, 1H), 2.82-2.69 (m, 1H), 2.36-2.26 (m, 1H); ¹³C NMR (126 MHz, DMSO) δ 168.38, 157.66, 156.88, 143.84, 141.13, 138.66, 136.38, 134.76, 132.01, 131.67, 130.75, 129.58, 127.50, 123.38, 121.10, 116.54, 115.25, 114.60, 47.46, 32.19, 31.15, 26.15; HRMS (ESI) m/z calcd for C₂H₂₁N₄O₃ [M+H]⁺ 461.1614, found 461.1619.

### 2-Chloro-4-(8,9,10,11-tetrahydro-3H-pyrazolo[4,3-a]phenanthridin-7-yl)phenol

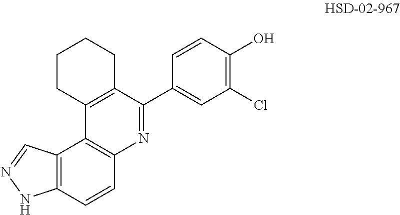 [SMILES: Oc1ccc(-c2nc3ccc4[nH]ncc4c3c3c2CCCC3)cc1Cl]

Method A: Off-white solid (191 mg, 55%). ¹H NMR (500 MHz, DMSO-d₆) δ 11.20 (s, 1H), 8.74 (s, 1H), 8.25-8.12 (m, 2H), 7.80 (d, J=2.1 Hz, 1H), 7.56 (dd, J=8.4, 2.2 Hz, 1H), 7.30 (d, J=8.4 Hz, 1H), 3.40 (d, J=6.7 Hz, 2H), 2.81 (t, J=6.1 Hz, 2H), 2.07-1.98 (m, 2H), 1.81-1.75 (m, 2H), ¹³C NMR (126 MHz, DMSO) δ 155.92, 152.58, 149.60, 135.00, 131.94, 131.83, 130.50, 123.67, 122.81, 120.32, 120.19, 116.98, 114.53, 30.78, 28.06, 21.65, 21.61; HRMS (ESI) m/z calcd for C20H17CIN₃O [M+H]⁺ 350.1060, found 350.1066.

### 4-(1-Bromo-8,9,10,11-tetrahydro-3H-pyrazolo[4,3-a]phenanthridin-7-yl)phenol

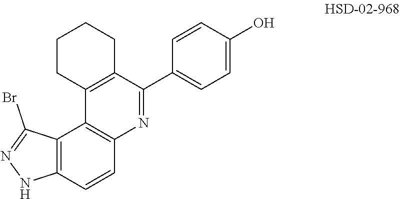 [SMILES: Oc1ccc(-c2nc3ccc4[nH]nc(Br)c4c3c3c2CCCC3)cc1]

Method A: Yellow solid (110 mg, 28%). Off-white solid (153 mg, 39%), ¹H NMR (500 MHz, Methanol-d₄) δ 8.18 (d, J=9.1 Hz, 1H), 8.06 (d, J=9.2 Hz, 1H), 7.57 (d, J=8.3 Hz, 2H), 7.07 (d, J=8.3 Hz, 2H), 3.99 (s, 2H), 2.97 (s, 2H), 1.97 (t, J=3.5 Hz, 4H), ¹³C NMR (126 MHz, MeOD) δ 160.37, 155.96, 151.79, 141.59, 135.37, 132.94, 130.83, 123.46, 121.93, 120.38, 119.36, 115.60, 114.56, 36.01, 27.02, 20.91, 20.80. ¹³C NMR (126 MHz, MeOD) δ 160.37, 155.96, 154.60, 151.79, 141.55, 135.37, 132.94, 130.83, 127.59, 123.46, 121.93, 120.38, 119.25, 115.60, 114.56, 36.01, 27.02, 20.91, 20.80; HRMS (ESI) m/z calcd for C20H17BrN₃O [M+H]⁺ 394.0555. found 394.0556.

### 7-(6-Chloropyridin-3-yl)-8,9,10,11-tetrahydro-3H-pyrazolo[4,3-a]phenanthridine

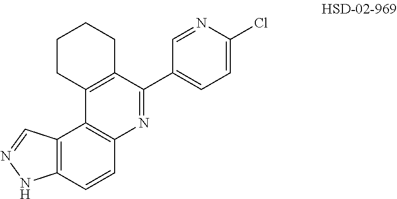 [SMILES: Clc1ccc(-c2nc3ccc4[nH]ncc4c3c3c2CCCC3)cn1]

Method A: White solid (210 mg, 63%). ¹H NMR (500 MHz, DMSO-d₆) δ 8.69 (d, J=2.4 Hz, 1H), 8.64 (s, 1H), 8.17 (dd, J=8.2, 2.5 Hz, 1H), 8.02-7.91 (m, 2H), 7.70 (d, J=8.2 Hz, 1H), 3.33 (t, J=6.6 Hz, 2H), 2.78 (t, J=6.2 Hz, 2H), 2.02-1.97 (m, 2H), 1.80-1.73 (m, 2H); HRMS (ESI) m/z calcd for C₁₉H₁₆ClN₄ [M+H]⁺ 335.1063, found 335.1063.

### 4-(8,9,10,11-Tetrahydro-3H-pyrazolo[4,3-a]phenanthridin-7-yl)-2-(trifluoromethyl)phenol

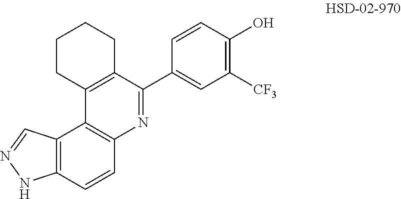 [SMILES: Oc1ccc(-c2nc3ccc4[nH]ncc4c3c3c2CCCC3)cc1C(F)(F)F]

Method A: white solid (233 mg, 61%). ¹H NMR (500 MHz, DMSO-d₆) δ 10.77 (s, 1H), 8.52 (s, 1H), 7.82 (d, J=2.2 Hz, 2H), 7.72-7.64 (m, 2H), 7.12 (d, J=8.3 Hz, 1H), 3.32-3.25 (m, 2H), 2.78 (t, J=6.1 Hz, 2H), 2.02-1.92 (m, 2H), 1.77-1.67 (m, 2H); ¹³C NMR (126 MHz, DMSO) δ 155.93, 155.43, 143.63, 142.52, 138.66, 136.33, 134.92, 131.78, 129.60, 129.39, 127.78, 127.73, 125.63 (q, J=273.42 Hz), 121.78, 116.87, 116.43, 115.61 (q, J=30.24 Hz), 114.39, 29.69, 29.00, 22.55; HRMS (ESI) m/z calcd for C₂₁H₁₇F₃N₃O [M+H]⁺ 384.1324, found 384.1329.

### 2-Methoxy-4-(8,9,10,11-tetrahydro-3H-pyrazolo[4,3-a]phenanthridin-7-yl)phenol

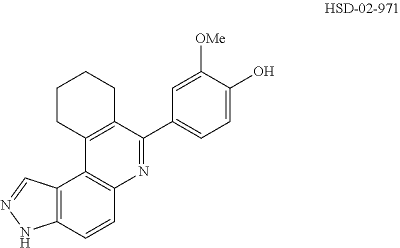 [SMILES: COc1cc(-c2nc3ccc4[nH]ncc4c3c3c2CCCC3)ccc1O]

Method A: White solid (231 mg, 67%). ¹H NMR (500 MHz, DMSO-d₆) δ 9.57 (s, 1H), 8.63 (s, 1H), 8.03 (s, 2H), 7.24 (d, J=2.1 Hz, 1H), 7.08 (dd, J=8.0, 2.0 Hz, 1H), 6.98 (d, J=8.0 Hz, 1H), 3.83 (s, 3H), 3.35 (s, 2H), 2.82 (t, J=6.2 Hz, 2H), 2.04-1.98 (m, 2H), 1.79-1.71 (m, 2H); ¹³C NMR (126 MHz, DMSO) δ 148.62, 147.81, 138.57, 135.55, 131.11, 126.48, 123.12, 122.31, 115.71, 114.34, 56.35, 30.47, 28.55, 22.05; HRMS (ESI) m/z calcd for C₂₁H₂₀N₃O₂ [M+H]⁺ 346.1556, found 346.1562.

### 3-Fluoro-4-(8,9,10,11-tetrahydro-3H-pyrazolo[4,3-a]phenanthridin-7-yl)phenol

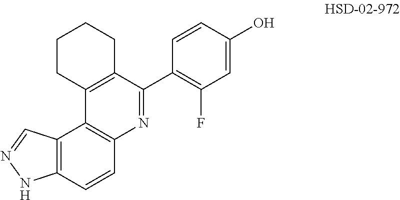 [SMILES: Oc1ccc(-c2nc3ccc4[nH]ncc4c3c3c2CCCC3)c(F)c1]

Method A: White solid (186 mg, 56%). ¹H NMR (500 MHz, DMSO-d₆) δ 8.57 (s, 1H), 7.84 (dd, J=9.0, 0.9 Hz, 1H), 7.79 (d, J=9.1 Hz, 1H), 7.22 (t, J=8.6 Hz, 1H), 6.72 (dd, J=8.3, 2.3 Hz, 1H), 6.66 (dd, J=11.8, 2.3 Hz, 1H), 3.30 (t, J=6.6 Hz, 2H), 2.61 (t, J=6.0 Hz, 2H), 2.00-1.91 (m, 2H), 1.79-1.69 (m, 2H); ¹³C NMR (126 MHz, DMSO) δ 161.25 (J=245.11 Hz), 159.59, 159.50, 152.73, 143.80, 142.04, 132.20, 132.16, 130.53, 129.53, 122.03, 119.48, 119.35, 116.25, 112.08, 102.89, 102.70, 29.48, 27.50, 22.64, 22.24; HRMS (ESI) m/z calcd for C₂₀H₁₇FN₃O [M+H]⁺ 334.1356, found 334.1360.

### 4-(9-Methyl-8,9,10,11-tetrahydro-3H-pyrazolo[4,3-a]phenanthridin-7-yl)phenol

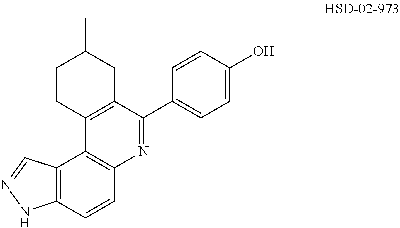 [SMILES: CC1CCc2c(c(-c3ccc(O)cc3)nc3ccc4[nH]ncc4c23)C1]

Method A: Off-white solid (223 mg, 68%). ¹H NMR (500 MHz, Methanol-d₄) δ 8.81 (s, 1H), 8.22 (d, J=9.3 Hz, 1H), 8.02 (d, J=9.3 Hz, 1H), 7.58 (dd, J=8.5, 1.9 Hz, 2H), 7.07 (dd, J=8.6, 1.9 Hz, 2H), 3.75-3.68 (m, 1H), 3.56 (dt, J=18.6, 8.3 Hz, 1H), 2.91 (dt, J=17.3, 2.7 Hz, 1H), 2.65 (dd, J=17.1, 10.6 Hz, 1H), 2.38-2.21 (m, 1H), 1.99-1.85 (m, 1H), 1.79-1.64 (m, 1H), 1.14 (dd, J=6.5, 1.8 Hz, 3H); ¹³C NMR (126 MHz, MeOD) δ 160.23, 153.67, 151.60, 134.75, 132.00, 130.90, 124.14, 123.00, 123.00, 122.62, 121.61, 119.36, 119.08, 116.55, 115.54, 115.03, 35.98, 30.99, 29.34, 27.83, 20.20; HRMS (ESI) m/z calcd for C₂₁H₂₀N₃O [M+H]⁺ 330.1606. found 330.1609.

### 4-(3,8,9,10-Tetrahydrocyclopenta[c]pyrazolo[4,3-f]quinolin-7-yl)phenol

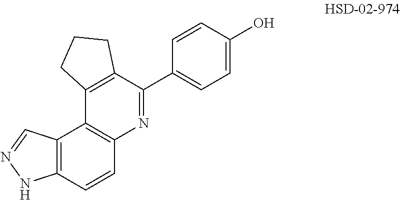 [SMILES: Oc1ccc(-c2nc3ccc4[nH]ncc4c3c3c2CCC3)cc1]

Method A: Off-white solid (168 mg, 56%). ¹H NMR (500 MHz, DMSO-d₆) δ 8.42 (s, 1H), 7.85 (d, J=9.2 Hz, 1H), 7.81 (dd, J=9.1, 0.9 Hz, 1H), 7.74 (d, J=8.6 Hz, 2H), 6.89 (d, J=8.6 Hz, 2H), 3.41 (t, J=7.6 Hz, 2H), 3.21 (t, J=7.4 Hz, 2H), 2.21 (p, J=7.6 Hz, 2H); ¹³C NMR (126 MHz, DMSO) δ 158.20, 152.26, 149.32, 144.91, 135.59, 131.44, 130.38, 129.22, 118.73, 116.70, 115.46, 33.48, 33.40, 24.97; HRMS (ESI) m/z calcd for C₁₉H₁₆N₃O [M+H]⁺ 302.1293, found 302.1295.

### 7-(4-Hydroxyphenyl)-8,9,10,11-tetrahydro-3H-pyrazolo[4,3-a]phenanthridine-9-carbonitrile

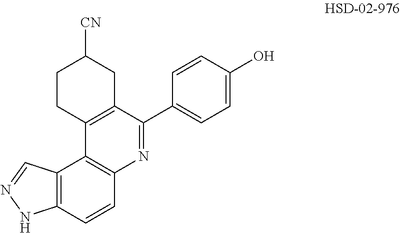 [SMILES: N#CC1CCc2c(c(-c3ccc(O)cc3)nc3ccc4[nH]ncc4c23)C1]

Method A: Yellow solid (207 mg, 61%). ¹H NMR (500 MHz, DMSO-d₆) δ 10.41 (s, 1H), 8.76 (s, 1H), 8.21 (d, J=9.2 Hz, 1H), 8.17 (d, J=9.2 Hz, 1H), 7.62 (d, J=8.2 Hz, 2H), 7.07 (d, J=8.2 Hz, 2H), 3.58-3.48 (m, 2H), 3.45-3.36 (m, 1H), 3.28-3.12 (m, 2H), 2.43-2.34 (m, 1H), 2.34-2.24 (m, 1H), ¹³C NMR (126 MHz, DMSO) δ 157.87, 156.56, 144.05, 140.61, 138.62, 136.16, 131.33, 130.95, 129.54, 125.63, 122.86, 120.97, 116.21, 115.29, 114.74, 31.73, 27.64, 25.08, 24.57; HRMS (ESI) m/z calcd for C₂₁H₁₇N₄O [M+H]⁺ 341.1402, found 341.1410.

### 2-((Dimethylamino)methyl)-4-(8,9,10,11-tetrahydro-3H-pyrazolo[4,3-a]phenanthridin-7-yl)phenol

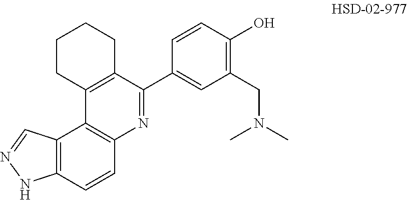 [SMILES: CN(C)Cc1cc(-c2nc3ccc4[nH]ncc4c3c3c2CCCC3)ccc1O]

Method A: Yellow solid (148 mg, 40%). ¹H NMR (500 MHz, Methanol-d₄) δ 8.63 (s, 1H), 7.92 (s, 2H), 7.61-7.55 (m, 2H), 7.12 (d, J=8.1 Hz, 1H), 4.42 (s, 2H), 3.43 (d, J=6.2 Hz, 2H), 2.93 (s, 6H), 2.85 (t, J=6.2 Hz, 2H), 2.13-2.08 (m, 2H), 1.91-1.82 (m, 2H); HRMS (ESI) m/z calcd for C₂₃H₂₅N₄O [M+H]⁺ 373.2028, found 373.2033.

### 7-(6-Fluoropyridin-3-yl)-8,9,10,11-tetrahydro-3H-pyrazolo[4,3-a]phenanthridine

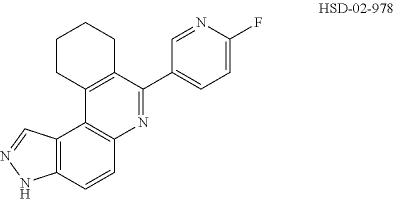 [SMILES: Fc1ccc(-c2nc3ccc4[nH]ncc4c3c3c2CCCC3)cn1]

Method A: Pale yellow solid (181 mg, 57%). ¹H NMR (500 MHz, Methanol-d₄) δ 8.51 (s, 1H), 8.40 (d, J=2.4 Hz, 1H), 8.16 (td, J=8.0, 2.5 Hz, 1H), 7.83 (s, 2H), 7.22 (dd, J=8.4, 2.4 Hz, 1H), 3.27 (t, J=6.6 Hz, 2H), 2.76 (t, J=6.2 Hz, 2H), 2.09-2.02 (m, 2H), 1.86-1.78 (m, 2H); ¹³C NMR (126 MHz, MeOD) δ 164.30, 162.39, 152.26, 147.56, 147.45, 144.47, 142.82, 142.68, 142.61, 133.99, 129.83, 127.94, 122.52, 115.69, 108.96, 108.67, 29.58, 28.25, 21.91, 21.87; HRMS (ESI) m/z calcd for C₁₉H₁₆FN₄ [M+H]⁺ 319.1359, found 319.1366.

### 2-Iodo-4-(8,9,10,11-tetrahydro-3H-pyrazolo[4,3-a]phenanthridin-7-yl)phenol

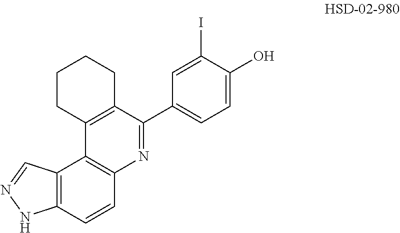 [SMILES: Oc1ccc(-c2nc3ccc4[nH]ncc4c3c3c2CCCC3)cc1I]

Method A: White solid (198 mg, 45%). ¹H NMR (500 MHz, Methanol-d₄) δ 8.87 (s, 1H), 8.28 (d, J=9.2 Hz, 1H), 8.11 (d, J=2.2 Hz, 1H), 8.06 (d, J=9.2 Hz, 1H), 7.60 (dd, J=8.4, 2.2 Hz, 1H), 7.10 (d, J=8.4 Hz, 1H), 3.65 (t, J=6.4 Hz, 2H), 2.95 (t, J=6.2 Hz, 2H), 2.31-2.09 (m, 2H), 2.03-1.69 (m, 2H); ¹³C NMR (126 MHz, MeOD) δ 159.64, 154.77, 153.68, 149.57, 140.11, 134.50, 132.47, 130.75, 129.17, 123.97, 123.61, 118.95, 114.86, 114.28, 109.63, 83.68, 30.98, 27.82, 21.28, 21.20; HRMS (ESI) m/z calcd for C₂₀H₁₇IN₃O [M+H]⁺ 442.0416, found 442.0416.

### 4-(8,9,10,11-Tetrahydro-3H-pyrazolo[4,3-a]phenanthridin-7-yl)benzoic acid

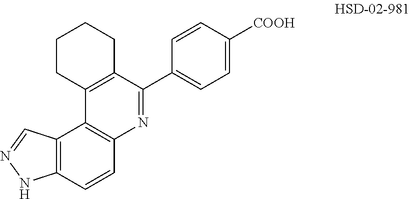 [SMILES: O=C(O)c1ccc(-c2nc3ccc4[nH]ncc4c3c3c2CCCC3)cc1]

Method A: Yellow solid (172 mg, 50%). ¹H NMR (500 MHz, DMSO-d₆) δ 8.62 (s, 1H), 8.12-8.08 (m, 2H), 8.02 (d, J=9.1 Hz, 1H), 7.95 (d, J=9.1 Hz, 1H), 7.80-7.73 (m, 2H), 3.29 (t, J=6.5 Hz, 2H), 2.75-2.68 (m, 2H), 2.00-1.94 (m, 2H), 1.76-1.66 (m, 2H), ¹³C NMR (126 MHz, DMSO) δ 167.42, 152.75, 148.00, 140.96, 139.42, 135.00, 131.73, 130.39, 130.19, 129.66, 129.59, 124.86, 122.74, 118.09, 115.38, 30.20, 28.32, 22.04, 21.92; HRMS (ESI) m/z calcd for C₂₁H₁₈N₃O₂ [M+H]⁺ 344.1399, found 344.1401.

### 4-(8,9-Dihydro-3H-cyclobuta[c]pyrazolo[4,3-f]quinolin-7-yl)phenol

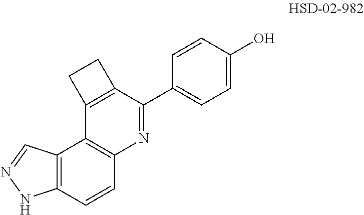

Method A: Off-white solid (152 mg, 53%). ¹H NMR (500 MHz, DMSO-d₆) δ 9.80 (s, 1H), 8.32 (s, 1H), 8.01 (dd, J=8.6, 1.4 Hz, 2H), 7.82 (d, J=9.5 Hz, 1H), 7.78 (d, J=9.1 Hz, 1H), 6.91 (dd, J=8.6, 1.4 Hz, 2H), 3.73-3.64 (m, 2H), 3.62-3.54 (m, 2H); ¹³C NMR (126 MHz, DMSO) δ 159.12, 149.75, 148.04, 144.97, 137.96, 136.56, 134.42, 130.02, 129.03, 128.87, 116.88, 116.11, 115.24, 114.32, 32.33, 30.24; HRMS (ESI) m/z calcd for C₁₈H₁₄N₃O [M+H]+ 288.1137, found 288.1139.

### 4-(3,8-Dihydro-2H-furo[3,2-c]pyrazolo[4,3-f]quinolin-4-yl)phenol

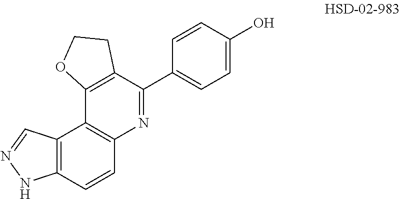

Method B: Off-white solid (127 mg, 42%). ¹H NMR (500 MHz, Methanol-d₄) δ 8.72 (s, 1H), 8.61 (s, 1H), 7.90 (d, J=9.2 Hz, 1H), 7.84 (d, J=9.2 Hz, 1H), 7.40 (d, J=8.5 Hz, 2H), 6.94 (d, J=8.5 Hz, 2H), 3.74 (t, J=7.0 Hz, 2H), 3.10 (t, J=7.0 Hz, 2H); HRMS (ESI) m/z calcd for C₁₈H₁₄N₃O₂ [M+H]⁺ 304.1086, found 304.1087.

### 7-(1H-Indazol-5-yl)-8,9,10,11-tetrahydro-3H-pyrazolo[4,3-a]phenanthridine

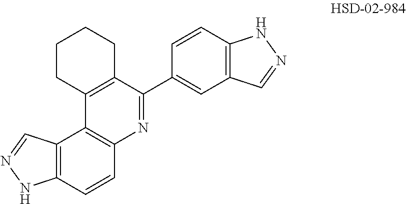

Method A: Off-white solid (224 mg, 65%). ¹H NMR (500 MHz, DMSO-d₆) δ 8.56 (s, 1H), 8.12 (s, 1H), 7.92 (s, 1H), 7.83 (d, J=5.8 Hz, 2H), 7.61 (d, J=8.6 Hz, 1H), 7.55 (d, J=8.6 Hz, 1H), 3.32-3.26 (m, 2H), 2.78 (t, J=6.1 Hz, 2H), 2.01-1.92 (m, 2H), 1.74-1.65 (m, 2H); ¹³C NMR (126 MHz, DMSO) δ 157.30, 143.71, 142.28, 139.80, 138.66, 136.27, 134.45, 133.55, 129.66, 129.54, 128.17, 123.07, 121.71, 121.43, 116.44, 114.31, 109.93, 29.69, 29.22, 22.64, 22.59; HRMS (ESI) m/z calcd for C₂₁H₁₈N₅[M+H]⁺ 340.1562, found 340.1565.

### 7-(1H-Indazol-6-yl)-8,9,10,11-tetrahydro-3H-pyrazolo[4,3-a]phenanthridine

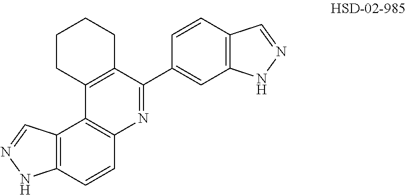

Method A: Off-white solid (176 mg, 52%). ¹H NMR (500 MHz, DMSO-d₆) δ 8.52 (s, 1H), 7.87-7.77 (m, 2H), 7.61 (d, J=1.6 Hz, 1H), 7.48 (d, J=8.2 Hz, 1H), 7.38 (dd, J=8.2, 1.7 Hz, 1H), 3.27 (d, J=7.7 Hz, 2H), 2.75 (t, J=6.0 Hz, 2H), 1.97 (qd, J=6.5, 4.2, 2.9 Hz, 2H), 1.79-1.67 (m, 2H); ¹³C NMR (126 MHz, DMSO) δ 155.27, 143.48, 142.61, 138.71, 138.01, 136.36, 133.74, 131.74, 129.55, 129.36, 125.92, 122.00, 116.36, 114.53, 111.49, 110.02, 29.64, 28.80, 22.52, 22.44; HRMS (ESI) m/z calcd for C₂₁H₁₈N₅ [M+H]⁺ 340.1562, found 340.1565.

### 5-(8,9,10,11-Tetrahydro-3H-pyrazolo[4,3-a]phenanthridin-7-yl)-1H-benzo[d]imidazol-2(3H)-one

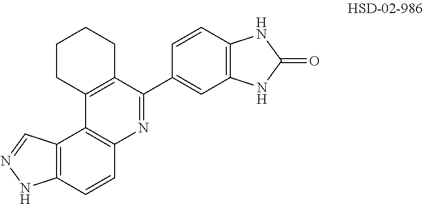

Method A: Off-white solid (178 mg, 50%). ¹H NMR (500 MHz, DMSO-d₆) δ 8.55 (s, 1H), 7.84 (d, J=9.1 Hz, 1H), 7.78 (d, J=9.1 Hz, 1H), 7.16-7.09 (m, 2H), 7.00 (d, J=7.8 Hz, 1H), 3.28 (t, J=6.5 Hz, 2H), 2.77 (t, J=6.1 Hz, 2H), 2.00-1.95 (m, 2H), 1.73-1.66 (m, 2H); ¹³C NMR (126 MHz, DMSO) δ 157.16, 156.09, 143.72, 142.21, 139.73, 134.99, 133.77, 130.00, 129.94, 129.53, 129.35, 122.16, 121.65, 116.30, 115.27, 109.89, 108.18, 29.70, 29.28, 22.65, 22.62; HRMS (ESI) m/z calcd for C₂₂H₁₇N₄O₂ [M+H]⁺ 369.1352, found 369.1352.

### 4-(8,9,10,11-Tetrahydro-1H-pyrazolo[3,4-a]phenanthridin-7-yl)phenol

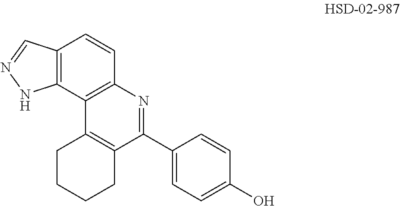 [SMILES: Oc1ccc(-c2nc3ccc4cn[nH]c4c3c3c2CCCC3)cc1]

Method A: Off-white solid (126 mg, 40%). ¹H NMR (500 MHz, DMSO-d₆) δ 9.63 (s, 1H), 8.14 (s, 1H), 7.77 (d, J=8.9 Hz, 1H), 7.58 (d, J=9.0 Hz, 1H), 7.43 (d, J=8.5 Hz, 2H), 6.88 (d, J=8.6 Hz, 2H), 3.17 (t, J=6.6 Hz, 2H), 2.73 (t, J=6.1 Hz, 2H), 1.88 (qd, J=7.8, 6.4, 4.5 Hz, 2H), 1.73-1.63 (m, 2H); ¹³C NMR (126 MHz, DMSO) δ 158.72, 157.79, 142.88, 138.02, 134.71, 134.54, 131.87, 130.95, 129.09, 124.62, 121.76, 119.80, 116.13, 115.13, 28.93, 26.48, 22.74, 22.32; HRMS (ESI) m/z calcd for C₂₀)H₁₈N₃O [M+H]⁺ 316.1449, found 316.1458.

### 5-(8,9,10,11-Tetrahydro-3H-pyrazolo[4,3-a]phenanthridin-7-yl)pyrimidine-2,4(1H,3H)-dione

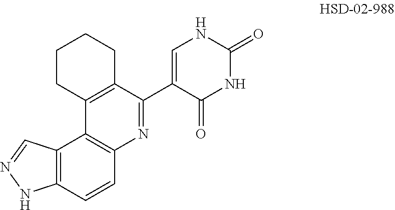

Method A: Off-white solid (173 mg, 52%). ¹H NMR (500 MHz, Methanol-d₄) δ 8.77 (s, 1H), 8.18-8.10 (m, 1H), 8.02-7.85 (m, 1H), 7.74-7.17 (m, 2H), 3.60-3.48 (m, 2H), 3.02-2.89 (m, 2H), 2.20-2.09 (m, 1H), 2.04-1.90 (m, 1H); HRMS (ESI) m/z calcd for C₁₈H₁₆N₅O₂ [M+H]⁺ 334.1304, found 334.1305.

### 2-Methyl-6-(8,9,10,11-tetrahydro-3H-pyrazolo[4,3-a]phenanthridin-7-yl)benzo[d]thiazole

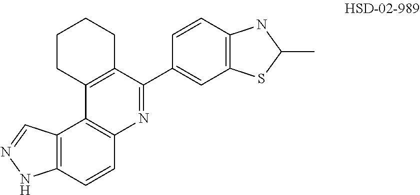

Method A: Off-white solid (207 mg, 56%). ¹H NMR (500 MHz, DMSO-d₆) δ 8.59 (s, 1H), 8.22 (d, J=1.6 Hz, 1H), 7.97 (d, J=8.3 Hz, 1H), 7.86 (dd, J=9.0, 0.9 Hz, 1H), 7.82 (d, J=9.1 Hz, 1H), 7.65 (dd, J=8.3, 1.7 Hz, 1H), 3.36-3.33 (m, 3H), 2.83 (s, 3H), 2.81-2.78 (m, 2H), 2.04-1.98 (m, 2H), 1.78-1.70 (m, 2H); ¹³C NMR (126 MHz, DMSO) δ 168.24, 156.38, 152.90, 143.77, 142.53, 137.87, 135.52, 129.61, 129.48, 127.80, 122.99, 121.99, 121.67, 118.44, 116.26, 29.71, 29.02, 22.62, 22.54, 20.33; HRMS (ESI) m/z calcd for C₂₂H₁₉N₄S [M+H]⁺ 371.1330, found 371.1333.

### 3,5-Difluoro-4-(8,9,10,11-tetrahydro-3H-pyrazolo[4,3-a]phenanthridin-7-yl)phenol

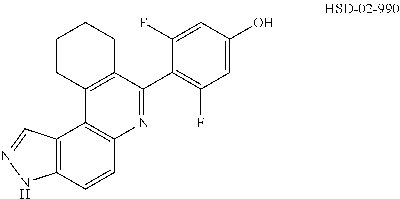

Method A: While solid (168 mg, 48%). ¹H NMR (500 MHz, DMSO-d₆) δ 8.52 (s, 1H), 7.84 (d, J=9.0 Hz, 1H), 7.80 (d, J=9.5 Hz, 1H), 7.61 (d, J=1.6 Hz, 1H), 7.48 (d, J=8.2 Hz, 1H), 7.38 (dd, J=8.2, 1.7 Hz, 1H), 3.27 (d, J=7.7 Hz, 2H), 2.75 (t, J=6.0 Hz, 2H), 1.99-1.92 (m, 2H), 1.76-1.65 (m, 2H); ¹³C NMR (126 MHz, DMSO) δ 155.27, 143.48, 142.96, 142.76, 142.61, 138.71, 138.01, 136.36, 133.74, 131.74, 129.55, 129.36, 125.92, 122.00, 116.36, 114.53, 111.49, 110.02, 29.64, 28.80, 22.52, 22.44; HRMS (ESI) m/z calcd for C₂₀H₁₆FN₃O [M+H]⁺ 352.1261, found 352.1266.

### 7-(4-Hydroxyphenyl)-8,9,10,11-tetrahydro-3H-pyrazolo[4,3-a]phenanthridine-9-carboxylic acid

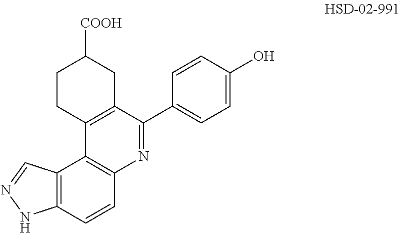

Method A: Off-white solid (180 mg, 50%). ¹H NMR (500 MHz, DMSO-d₆) δ 8.56 (s, 1H), 7.83 (d, J=9.1 Hz, 1H), 7.78 (d, J=9.1 Hz, 1H), 7.38 (d, J=8.5 Hz, 2H), 6.87 (d, J=8.5 Hz, 2H), 3.48-3.39 (m, 1H), 3.36-3.23 (m, H), 3.05-2.97 (m, 1H), 2.95-2.87 (m, 1H), 2.62-2.56 (m, 1H), 2.38-2.30 (m, 1H), 2.01-1.91 (m, 1H); ¹³C NMR (126 MHz, DMSO) δ 177.11, 157.71, 156.96, 143.77, 141.52, 131.87, 130.83, 129.55, 128.25, 121.22, 116.27, 115.19, 31.66, 29.18, 25.49; HRMS (ESI) m/z calcd for C₂₁H₁₈N₃O₃ [M+H]⁺ 360.1348, found 360.1351.

### 5-(8,9,10,11-Tetrahydro-3H-pyrazolo[4,3-a]phenanthridin-7-yl)benzo[d]thiazol-2-amine

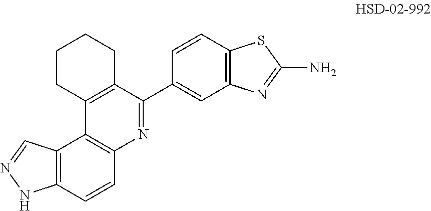

Method A: Pale yellow solid (137 mg, 37%). ¹H NMR (500 MHz, DMSO-d₆) δ 8.55 (s, 1H), 7.88-7.78 (m, 3H), 7.55 (s, 2H), 7.45-7.36 (m, 2H), 3.32 (s, 2H), 2.82 (t, J=6.1 Hz, 2H), 1.99 (qq, J=5.2, 2.7 Hz, 2H), 1.74 (dp, J=9.1, 3.2, 2.8 Hz, 2H); ¹³C NMR (126 DMSO) δ 167.61, 156.90, 152.94, 143.53, 142.39, 138.63, 136.32, 133.93, 131.17, 129.60, 127.25, 122.13, 121.64, 117.32, 116.50, 114.34, 29.75, 29.18, 22.66, 22.62; HRMS (ESI) m/z calcd for C₂₁H₁₈N₅S [M+H]⁺ 372.1283, found 372.1285.

### 4-(3,8,10,11-Tetrahydropyrano[3,4-c]pyrazolo[4,3-f]quinolin-7-yl)phenol

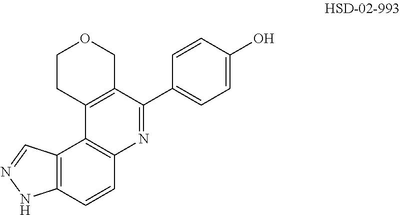 [SMILES: Oc1ccc(-c2nc3ccc4[nH]ncc4c3c3c2COCC3)cc1]

Method A: Off-white solid (206 mg, 65%). ¹H NMR (500 MHz, DMSO-d₆) δ 9.69 (s, 1H), 8.52 (s, 1H), 7.85 (q, J=9.2 Hz, 2H), 7.40 (d, J=8.5 Hz, 2H), 6.87 (d, J=8.6 Hz, 2H), 4.77 (s, 2H), 4.11 (t, J=5.9 Hz, 2H), 3.34 (d, J=6.8 Hz, 2H); ¹³C NMR (126 MHz, DMSO) δ 158.09, 153.88, 144.20, 139.12, 138.55, 135.81, 130.66, 130.59, 129.53, 127.54, 120.80, 116.34, 115.40, 114.82, 67.17, 64.45, 28.70; HRMS (ESI) m/z calcd for C₁₉H₁₆N₃O₂ [M+H]⁺ 318.1243, found 318.1244.

### N-(7-(4-Hydroxyphenyl)-8,9,10,11-tetrahydro-3H-pyrazolo[4,3-a]phenanthridin-9-yl)acetamide

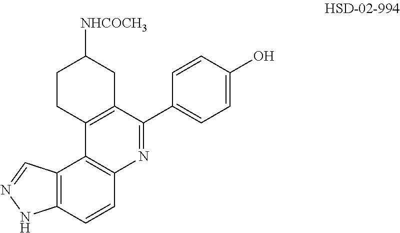

Method A: Pale brownish solid (220 mg, 59%). ¹H NMR 1500 MHz, DMSO-d₆) δ 8.57 (s, 1H), 7.97 (d, J=7.2 Hz, 1H), 7.84 (dd, J=9.0, 0.9 Hz, 1H), 7.79 (d, J=9.1 Hz, 1H), 7.40-7.35 (m, 2H), 6.89-6.80 (m, 2H), 3.96-3.88 (m, 1H), 3.52-3.46 (m, 1H), 3.34-3.29 (m, 1H), 2.98-2.90 (m, 1H), 2.75 (dd, J=16.6, 9.0 Hz, 1H), 2.24-2.16 (m, 1H), 1.95-1.83 (m, 1H), 1.77 (s, 3H); ¹³C NMR (126 MHz, DMSO) δ 169.29, 157.68, 157.02, 143.86, 141.35, 131.82, 130.82, 129.56, 127.39, 121.15, 116.35, 115.20, 44.69, 35.05, 28.48, 28.02, 23.15; HRMS (ESI) m/z calcd for C₂₂H₂₁N₄O₂ [M+H]⁺ 373.1665, found 373.1670.

### 4-(6,7,8,9-Tetrahydro-1H-pyrazolo[3,4-c]phenanthridin-5-yl)phenol

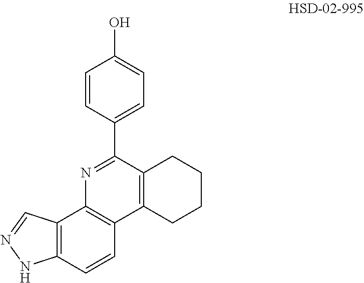 [SMILES: Oc1ccc(-c2nc3c(ccc4[nH]ncc43)c3c2CCCC3)cc1]

Method A: Pale brownish solid (132 mg, 42%). ¹H NMR (500 MHz, Methanol-d₄) δ 8.90 (s, 1H), 8.28 (dd, J=11.6, 5.5 Hz, 2H), 8.05 (t, J=8.7 Hz, 2H), 7.62-7.54 (m, 2H), 7.11-7.05 (m, 2H), 3.57 (t, J=6.4 Hz, 2H), 2.88 (t, J=6.2 Hz, 2H), 2.14-2.05 (m, 2H), 1.93-1.85 (m, 2H); ¹³C NMR (126 MHz, MeOD) δ 160.37, 156.23, 153.72, 132.09, 130.99, 130.27, 123.52, 122.38, 122.21, 115.50, 111.83, 27.76, 27.47, 21.48, 21.18; HRMS (ESI) m/z calcd for C₂₀H₁₈N₃O [M+H]⁺ 316.1449, found 316.1456.

### 4-(6,7,8,9-Tetrahydro-3H-pyrazolo[4,3-c]phenanthridin-5-yl)phenol

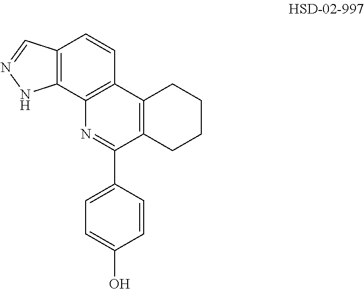 [SMILES: Oc1ccc(-c2nc3c(ccc4cn[nH]c43)c3c2CCCC3)cc1]

Method A: Pale yellow solid (161 mg, 51%). ¹H NMR (500 MHz, Methanol-d₄) δ 8.49 (s, 1H), 8.11 (d, J=9.2 Hz, 1H), 7.89 (d, J=9.2 Hz, 1H), 7.65-7.56 (m, 2H), 7.12-7.03 (m, 2H), 3.53 (t, J=6.5 Hz, 2H), 2.92 (t, J=6.3 Hz, 2H), 2.14-2.05 (m, 2H), 1.95-1.83 (m, 2H); ¹³C NMR (126 MHz, DMSO) δ 162.75, 159.17, 154.29, 147.97, 140.25, 139.21, 136.17, 134.68, 130.81, 128.84, 127.83, 124.74, 122.97, 122.65, 122.32, 115.57, 110.49, 110.49, 30.27, 28.70, 22.16, 22.08; HRMS (ESI) m/z calcd for C₂₀H₁₈N₃O [M+H]⁺ 316.1449, found 316.1459.

### 7-(1H-Pyrrolo[2,3-b]pyridin-5-yl)-8,9,10,11-tetrahydro-3H-pyrazolo[4,3-a]phenanthridine

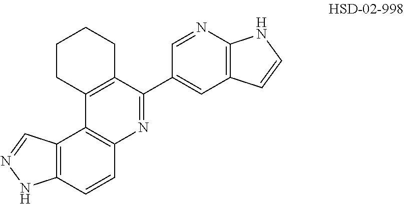 [SMILES: c1cc2cc(-c3nc4ccc5[nH]ncc5c4c4c3CCCC4)cnc2[nH]1]

Method A: Off-white solid (203 mg, 60%). ¹H NMR (500 MHz, DMSO-d₆) δ 12.09 (s, 1H), 8.78 (s, 1H), 8.54 (d, J=2.1 Hz, 1H), 8.37 (d, J=2.1 Hz, 1H), 8.17 (q, J=9.2 Hz, 2H), 7.65 (t, J=3.0 Hz, 1H), 6.61 (dd, J=3.5, 1.8 Hz, 1H), 3.51-3.44 (m, 2H), 2.86 (t, J=6.2 Hz, 2H), 2.09-2.01 (m, 2H), 1.86-1.73 (m, 2H); ¹³C NMR (126 MHz, DMSO) δ 151.20, 149.00, 143.45, 136.93, 132.02, 130.20, 128.42, 122.92, 122.10, 119.30, 115.16, 115.08, 101.14, 30.71, 28.46, 21.95, 21.88; HRMS (ESI) m/z calcd for C₂₁H₁₈N₅ [M+H]⁺ 340.1562, found 340.1569.

### 4-(8,9,10,11-Tetrahydro-1H-pyrazolol[3,4-a]phenanthridin-7-yl)phenol

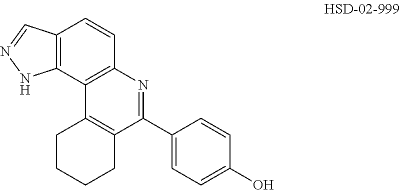 [SMILES: Oc1ccc(-c2nc3ccc4cn[nH]c4c3c3c2CCCC3)cc1]

Method A: Off-white solid (182 mg, 58%). ¹H NMR (500 MHz, DMSO-d₆) δ 8.31 (s, 1H), 7.87 (d, J=8.9 Hz, 1H), 7.51 (d, J=8.9 Hz, 1H), 7.39 (d, J=8.5 Hz, 2H), 6.85 (d, J=8.5 Hz, 2H), 3.50 (t, J=6.6 Hz, 2H), 2.79 (t, J=6.1 Hz, 2H), 1.97-1.92 (m, 2H), 1.77-1.70 (m, 2H); ¹³C NMR (126 MHz, DMSO) δ 158.40, 157.95, 146.70, 142.55, 131.73, 130.87, 128.50, 124.50, 122.19, 119.87, 116.93, 115.17, 29.97, 29.12, 22.56; HRMS (ESI) m/z calcd for C₂₀H₁₈N₃O [M+H]⁺ 316.1449, found 316.1458.

### 3,5-Dimethyl-4-(8,9,10,11-tetrahydro-3H-pyrazolo[4,3-a]phenanthridin-7-yl)isoxazole

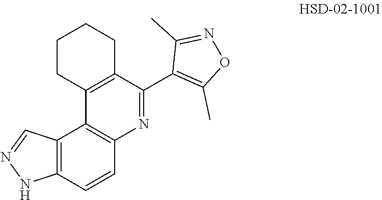 [SMILES: Cc1noc(C)c1-c1nc2ccc3[nH]ncc3c2c2c1CCCC2]

Method A: White solid (229 mg, 72%). ¹H NMR (500 MHz, Methanol-d₄) δ 8.92 (s, 1H), 8.35 (d, J=9.3 Hz, 1H), 8.06 (dd, J=9.2, 2.4 Hz, 1H), 3.71-3.64 (m, 2H), 2.86-2.77 (m, 2H), 2.45 (s, 3H), 2.25-2.20 (m, 5H), 2.06-1.98 (m, 2H); ¹³C NMR (126 MHz, MeOD) δ 170.18, 158.64, 155.86, 140.72, 140.00, 135.64, 134.42, 134.28, 124.70, 121.79, 118.93, 114.70, 108.54, 30.94, 26.68, 21.17, 20.88, 10.39, 8.99; HRMS (ESI) m/z calcd for C₁₉H₁₉N₄O [M+H]⁺ 319.1559, found 319.1563.

### 7-(4-Hydroxyphenyl)-8,9,10,11-tetrahydro-3H-pyrazolo[4,3-a]phenanthridine-1-carbonitrile

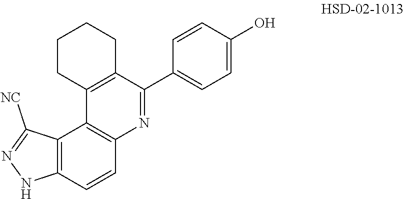 [SMILES: N#Cc1n[nH]c2ccc3nc(-c4ccc(O)cc4)c4c(c3c12)CCCC4]

Method A: White solid (210 mg, 61%). ¹H NMR 1500 MHz, Methanol-d₄) δ 8.32 (d, J=9.3 Hz, 1H), 8.20 (d, J=9.2 Hz, 1H), 7.62 (d, J=8.6 Hz, 2H), 7.09 (d, J=8.5 Hz, 2H), 3.91 (t, J=6.3 Hz, 2H), 3.00 (t, J=6.3 Hz, 2H), 2.24-2.09 (m, 2H), 2.01-1.90 (m, 2H). ¹³C NMR (126 MHz, MeOD) δ 160.62, 155.55, 153.29, 139.97, 135.54, 133.16, 130.98, 122.63, 121.71, 121.08, 119.18, 115.88, 115.65, 115.28, 33.30, 27.88, 20.92, 20.63; HRMS (ESI) m/z calcd for C₂₁H₂₁N₄O [M+H]⁺ 345.1715, found 345.1720.

### tert-Butyl (6-(8,9,10,11-tetrahydro-3H-pyrazolo[4,3-a]phenanthridin-7-yl)pyridin-2-yl)carbamate

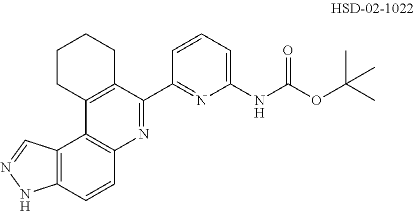 [SMILES: CC(C)(C)OC(=O)Nc1cccc(-c2nc3ccc4[nH]ncc4c3c3c2CCCC3)n1]

Method A: White solid (270 mg, 65%). ¹H NMR (500 MHz, Methanol-d₄) δ 8.79 (s, 1H), 8.23 (d, J=9.3 Hz, 1H), 8.12-8.05 (m, 3H), 7.65 (dd, J=5.7, 2.6 Hz, 1H), 3.53 (d, J=6.0 Hz, 2H), 3.08 (t, J=6.1 Hz, 2H), 2.21-2.14 (m, 2H), 1.99-1.91 (m, 2H), 1.58 (s, 9H); ¹³C NMR (126 MHz, MeOD) δ 154.62, 153.09, 153.05, 147.37, 146.93, 140.49, 139.57, 134.87, 134.39, 132.10, 124.03, 120.98, 119.75, 119.68, 114.62, 114.29, 80.98, 30.97, 27.41, 27.15, 21.16, 21.06.

### 4-(5-Methoxy-8,9,10,11-tetrahydro-3H-pyrazolo[4,3-a]phenanthridin-7-yl)phenol

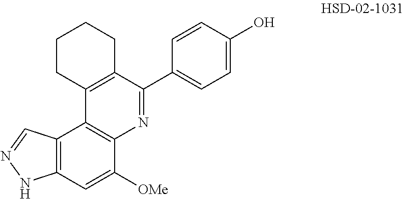 [SMILES: COc1cc2[nH]ncc2c2c3c(c(-c4ccc(O)cc4)nc12)CCCC3]

Method A: White solid (145 mg, 42%). ¹H NMR (500 MHz, DMSO-d₆) δ 10.16 (s, 1H), 8.60 (s, 1H), 7.53-7.39 (m, 3H), 7.03-6.92 (m, 2H), 4.05 (s, 3H), 3.37 (t, J=6.5 Hz, 2H), 2.75 (t, J=6.2 Hz, 2H), 1.99 (pd, J=6.4, 4.3, 2.6 Hz, 2H), 1.75 (dq, J=6.0, 2.9, 2.5 Hz, 2H); ¹³C NMR (126 MHz, DMSO) δ 159.52, 152.70, 150.67, 150.36, 140.21, 132.48, 131.68, 130.23, 125.17, 123.07, 121.30, 115.56, 109.83, 96.56, 56.90, 30.79, 28.48, 21.93, 21.80; HRMS (ESI) m/z calcd for C₂₁H₂₀N₃O₂ [M+H]⁺ 346.1556, found 346.1559.

### 4-(3-Methyl-8,9,10,11-tetrahydro-3H-pyrazolo[4,3-a]phenanthridin-7-yl)phenol

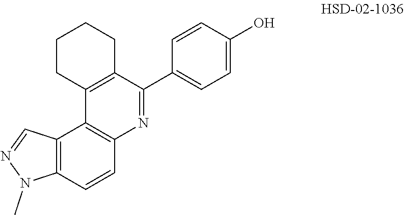 [SMILES: Cn1ncc2c3c4c(c(-c5ccc(O)cc5)nc3ccc21)CCCC4]

Method A: Off-white solid (208 mg, 63%). ¹H NMR (500 MHz, Methanol-d₄) δ 8.74 (s, 1H), 8.34 (d, J=9.2 Hz, 1H), 8.11 (d, J=9.3 Hz, 1H), 7.63-7.56 (m, 2H), 7.10-7.03 (m, 2H), 4.30 (s, 3H), 3.64 (t, J=6.4 Hz, 2H), 2.96 (t, J=6.2 Hz, 2H), 2.23-2.14 (m, 2H), 1.98-1.88 (m, 2H); ¹³C NMR (126 MHz, MeOD) δ 160.36, 154.36, 151.77, 138.31, 134.95, 134.11, 132.57, 130.94, 123.00, 122.30, 118.94, 118.25, 116.10, 115.57, 35.20, 30.96, 27.98, 21.33, 21.26.

### 7-(1H-Pyrazol-4-yl)-8,9,10,11-tetrahydro-3H-pyrazolo[4,3-a]phenanthridine

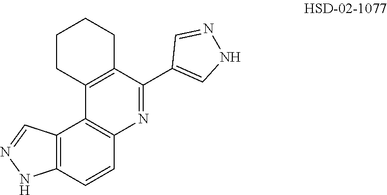 [SMILES: c1n[nH]cc1-c1nc2ccc3[nH]ncc3c2c2c1CCCC2]

Method A: White solid (220 mg, 76%). ¹H NMR (500 MHz, DMSO-d₆) δ 8.52 (s, 1H), 8.14 (s, 2H), 7.79 (d, J=5.4 Hz, 2H), 3.28 (d, J=6.7 Hz, 2H), 2.99 (t, J=6.1 Hz, 2H), 1.98 (ddt, J=9.0, 6.4, 3.2 Hz, 2H), 1.84 (dP, J=9.2, 3.4, 2.9 Hz, 2H); ¹³C NMR (126 MHz, DMSO) δ 149.72, 143.79, 142.11, 138.53, 136.11, 129.61, 128.83, 121.65, 121.03, 116.45, 114.22, 29.85, 28.67, 22.64, 22.48; HRMS (ESI) m/z calcd for C₁₇H₁₆N₅ [M+H]⁺ 290.1406, found 290.1406.

### 7-(1H-Indazol-5-yl)-8,9,10,11-tetrahydro-3H-pyrrolo[3,2-a]phenanthridine

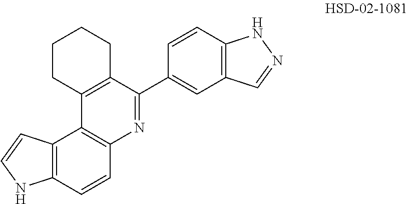 [SMILES: c1cc2c(ccc3nc(-c4ccc5[nH]ncc5c4)c4c(c32)CCCC4)[nH]1]

Method A: White solid (173 mg, 51%). ¹H NMR (500 MHz, DMSO-d₆) δ 11.76 (s, 1H), 8.12 (s, 1H), 7.91 (s, 1H), 7.80 (d, J=8.8 Hz, 1H), 7.66 (d, J=8.8 Hz, 1H), 7.61 (d, J=8.5 Hz, 1H), 7.54 (dd, J=8.5, 1.5 Hz, 1H), 7.48 (t, J=2.8 Hz, 1H), 7.11 (t, J=2.6 Hz, 1H), 3.37 (d, J=6.9 Hz, 2H), 2.77 (t, J=6.2 Hz, 2H), 1.99-1.91 (m, 2H), 1.73-1.64 (m, 2H); ¹³C NMR (126 MHz, DMSO) δ 156.34, 143.43, 142.27, 139.73, 134.40, 134.08, 133.20, 128.24, 127.74, 124.12, 123.87, 123.10, 122.38, 121.28, 120.41, 116.42, 109.84, 106.27, 30.01, 29.35, 22.91, 22.62; HRMS (ESI) m/z calcd for C₂₂H₁₉N₄ [M+H]⁺ 339.1610, found 339.1618.

### 7-(1H-Indol-2-yl)-8,9,10,11-tetrahydro-3H-pyrazolo[4,3-a]phenanthridine

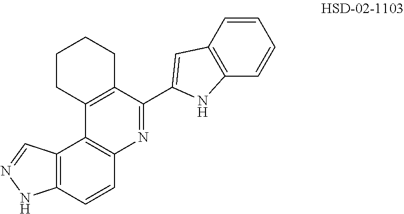 [SMILES: c1ccc2[nH]c(-c3nc4ccc5[nH]ncc5c4c4c3CCCC4)cc2c1]

Method A: Yellow solid (264 mg, 78%). ¹H NMR (500 MHz, DMSO-d₆) δ 12.12 (s, 1H), 8.76 (s, 1H), 8.38-8.08 (m, 2H), 7.70 (d, J=8.0 Hz, 1H), 7.57 (dd, J=8.2, 1.0 Hz, 1H), 7.30-7.21 (m, 2H), 7.09 (td, J=7.4, 6.9, 1.0 Hz, 1H), 3.45 (t, J=6.4 Hz, 2H), 3.18-3.15 (m, 2H), 2.11-1.99 (m, 2H), 1.92-1.81 (m, 2H); ¹³C NMR (126 MHz, DMSO) δ 143.78, 137.55, 134.89, 131.22, 130.49, 128.14, 124.23, 122.63, 121.72, 120.53, 119.45, 115.27, 112.54, 108.05, 30.84, 28.26, 21.91, 21.86, 21.86; HRMS (ESI) m/z calcd for C₂₂H₁₉N₄ [M+H]⁺ 339.1610, found 339.1618.

### 7-(1H-indol-3-yl)-8,9,10,11-tetrahydro-3H-pyrazolo[4,3-a]phenanthridine

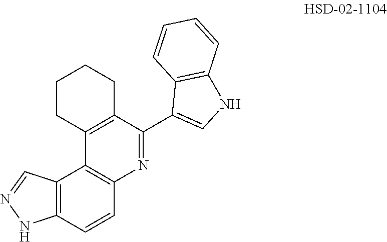 [SMILES: c1ccc2c(-c3nc4ccc5[nH]ncc5c4c4c3CCCC4)c[nH]c2c1]

Method A: White solid (183 mg, 54%). ¹H NMR (500 MHz, Methanol-d₄) δ 8.72 (s, 1H), 8.14 (d, J=8.7 Hz, 1H), 8.04-7.93 (m, 2H), 7.61-7.52 (m, 2H), 7.30-7.16 (m, 2H), 3.49 (t, J=6.2 Hz, 2H), 2.99 (t, J=6.1 Hz, 2H), 2.22-2.10 (m, 2H), 1.87 (q, J=5.7 Hz, 2H); ¹³C NMR (126 MHz, MeOD) δ 152.83, 147.51, 136.55, 135.07, 132.76, 128.62, 125.73, 122.77, 122.39, 120.99, 119.61, 118.86, 115.08, 112.11, 107.22, 30.80, 27.71, 21.46, 21.29; HRMS (ESI) m/z calcd for C₂₂H₁₉N₄ [M+H]⁺ 339.1610, found 339.1618.

### 7-Cyclopropyl-8,9,10,11-tetrahydro-3H-pyrazolo[4,3-a]phenanthridine

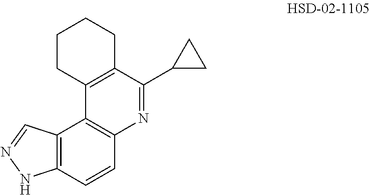 [SMILES: c1cc2[nH]ncc2c2c3c(c(C4CC4)nc12)CCCC3]

Method A: Off-white solid (181 mg, 69%). ¹H NMR (500 MHz, DMSO-d₆) δ 8.45 (s, 1H), 7.73 (d, J=9.0 Hz, 1H), 7.64 (d, J=9.1 Hz, 1H), 3.18 (q, J=5.3, 4.6 Hz, 2H), 2.94 (t, J=6.2 Hz, 2H), 2.21 (tt, J=8.1, 4.9 Hz, 1H), 1.96-1.88 (m, 2H), 1.86-1.81 (m, 2H), 1.10-0.99 (m, 2H), 0.97-0.87 (m, 2H); ¹³C NMR (126 MHz, DMSO) δ 157.71, 143.33, 140.97, 138.28, 135.75, 129.57, 129.38, 120.82, 116.55, 113.67, 29.49, 26.38, 22.40, 22.37, 13.67, 9.14; HRMS (ESI) m/z calcd for C₁₇H₁₈N₃ [M+H]⁺ 264.1501, found 264.1509.

### 2-(8,9,10,11-Tetrahydro-3H-pyrazolo[4,3-a]phenanthridin-7-yl)-4H-chromen-4-one

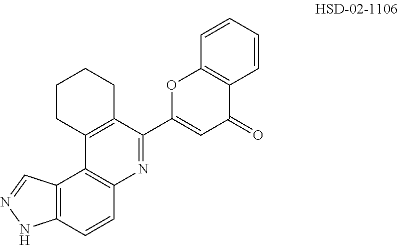 [SMILES: O=c1cc(-c2nc3ccc4[nH]ncc4c3c3c2CCCC3)oc2ccccc12]

Method A: Off-white solid (92 mg, 25%). ¹H NMR (500 MHz, DMSO-d₆) δ 8.88 (s, 1H), 8.85 (s, 1H), 8.26 (d, J=9.2 Hz, 1H), 8.23 (d, J=9.2 Hz, 1H), 8.17 (dd, J=8.0, 1.7 Hz, 1H), 7.95 (dt, J=8.7, 7.1 Hz, 1H), 7.85-7.83 (d, J=8.2 Hz, 1H), 7.62 (t, J=8.0 Hz 1H), 3.52 (t, J=6.4 Hz, 2H), 2.87 (t, J=6.3 Hz, 2H), 2.08-1.98 (m, 2H), 1.82 (dp, J=9.8, 3.9, 3.1 Hz, 2H); HRMS (ESI) m/z calcd for C₂₃H₁₈N₃O₂ [M+H]⁺ 368.1399, found 368.1400.

### 7-(Bicyclo[2.2.1]hept-5-en-2-yl)-8,9,10,11-tetrahydro-3H-pyrazolo[4,3-a]phenanthridine

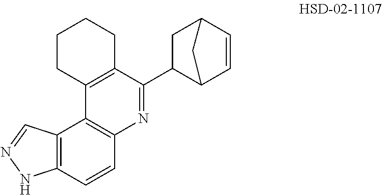 [SMILES: C1=CC2CC1CC2c1nc2ccc3[nH]ncc3c2c2c1CCCC2]

Method A: Off-white solid (110 mg, 34%). ¹H NMR (500 MHz, Methanol-d₄) δ 8.82 (s, 1H), 8.34 (d, J=9.3 Hz, 1H), 8.23 (d, J=9.2 Hz, 1H), 6.71 (dd, J=5.8, 3.2 Hz, 1H), 6.44-6.36 (m, 1H), 5.68 (dd, J=5.7, 2.9 Hz, 1H), 4.34-4.28 (m, 1H), 3.64-3.59 (m, 2H), 3.43 (s, 1H), 3.24 (d, J=4.8 Hz, 2H), 2.42 (ddd, J=12.9, 9.3, 3.6 Hz, 1H), 2.24-2.16 (m, 1H), 2.14-2.03 (m, 4H), 1.76 (d, J=8.4 Hz, 1H), 1.74-1.66 (m, 2H); HRMS (ESI) m/z calcd for C₂₃H₂₂N₃ [M+H]⁺ 316.1814, found 316.1819.

### 7-(1H-Indazol-5-yl)-1-iodo-8,9,10,11-tetrahydro-3H-pyrazolo[4,3-a]phenanthridine

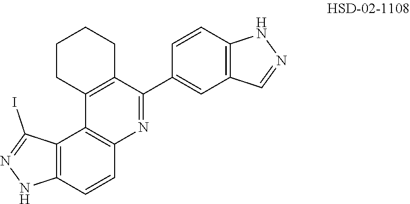 [SMILES: Ic1n[nH]c2ccc3nc(-c4ccc5[nH]ncc5c4)c4c(c3c12)CCCC4] [0350] Method A: Yellow solid (302 mg, 65%). ¹H NMR (500 MHz, DMSO-d₆) δ 8.13 (s, 1H), 7.94 (t, J=1.1 Hz, 1H), 7.79 (d, J=9.1 Hz, 1H), 7.76 (d, J=9.0 Hz, 1H), 7.62 (d, J=8.6 Hz, 1H), 7.55 (dd, J=8.6, 1.6 Hz, 1H), 3.70 (t, J=5.9 Hz, 2H), 2.82 (t, J=6.3 Hz, 2H), 1.76-1.72 (m, 4H); ¹³C NMR (126 MHz, DMSO) δ 157.35, 144.29, 143.76, 141.32, 139.91, 134.39, 133.08, 130.70, 129.56, 129.31, 128.08, 123.10, 121.70, 121.50, 120.04, 114.80, 110.12, 37.07, 27.57, 22.22, 22.04; HRMS (ESI) m/z calcd for C₂₁H₁₇IN₅[M+H]⁺ 466.0529, found 466.0535.

### 1-Bromo-7-(1H-indazol-5-yl)-8,9,10,11-tetrahydro-3H-pyrazolo[4,3-a]phenanthridine

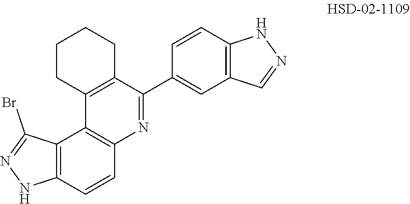 [SMILES: Brc1n[nH]c2ccc3nc(-c4ccc5[nH]ncc5c4)c4c(c3c12)CCCC4]

Method A: Off-white solid (234 mg, 56%). ¹H NMR (500 MHz, DMSO-d₆) δ 8.13 (d, J=1.0 Hz, 1H), 7.94-7.92 (m, 1H), 7.83-7.76 (m, 2H), 7.62 (dt, J=8.6, 1.0 Hz, 1H), 7.54 (dd, J=8.6, 1.6 Hz, 1H), 3.60 (t, J=5.9 Hz, 2H), 2.81 (t, J=6.2 Hz, 2H), 1.79-1.70 (m, 4H); ¹³C NMR (126 MHz, DMSO) δ 157.76, 144.25, 143.42, 141.60, 139.90, 134.39, 133.10, 131.25, 129.53, 128.02, 123.10, 121.51, 121.45, 115.33, 114.67, 110.13, 34.34, 27.88, 22.29, 22.10; HRMS (ESI) m/z calcd for C₁₉H₁₈N₅ [M+H]⁺ 316.1562, found 316.1555.

### N-(7-(1H-Indazol-5-yl)-8,9,10,11-tetrahydro-3H-pyrazolo[4,3-a]phenanthridin-9-yl)acetamide

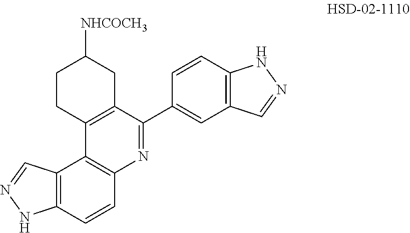

Method A: Off-white solid (277 mg, 70%). ¹H NMR (500 MHz, DMSO-d₆) δ 8.71 (s, 1H), 8.23 (s, 1H), 8.09 (q, J=7.1, 5.1 Hz, 4H), 7.74 (d, J=8.6 Hz, 1H), 7.63 (dd, J=8.6, 1.6 Hz, 1H), 4.04-3.93 (m, 1H), 3.57 (dt, J=18.7, 5.5 Hz, 1H), 3.45 (dt, J=17.9, 7.4 Hz, 1H), 2.96 (dd, J=16.8, 4.7 Hz, 1H), 2.80 (dd, J=16.7, 8.7 Hz, 1H), 2.27-2.18 (m, 1H), 2.04-1.91 (m, 1H), 1.75 (s, 3H); ¹³C NMR (126 MHz, DMSO) δ 169.40, 153.72, 140.39, 138.50, 134.76, 129.36, 127.67, 122.94, 122.22, 115.46, 110.75, 44.11, 34.25, 29.21, 27.36, 23.11; HRMS (ESI) m/z calcd for C₂₃H₂₁N₆O [M+H]⁺ 397.1777, found 397.1778.

### (7-(1H-Indazol-5-yl)-8,9,10,11-tetrahydro-3H-pyrazolo[4,3-a]phenanthridin-9-amine

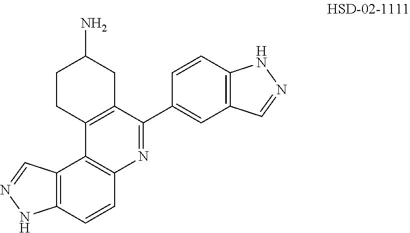 [SMILES: NC1CCc2c(c(-c3ccc4[nH]ncc4c3)nc3ccc4[nH]ncc4c23)C1]

Method A: Pale yellow solid (227 mg, 64%). ¹H NMR (500 MHz, DMSO-d₆) δ 8.80 (s, 1H), 8.60-8.51 (m, 3H), 8.27 (s, 1H), 8.24 (s, 2H), 8.16 (s, 1H), 7.80 (d, J=8.5 Hz, 1H), 7.65 (dd, J=8.6, 1.6 Hz, 1H), 3.74-3.65 (m, 1H), 3.54 (q, J=9.8, 9.2 Hz, 2H), 3.15-3.09 (m, 2H), 2.55-2.51 (m, 1H), 2.20-2.10 (m, 1H); ¹³C NMR (126 MHz, DMSO) δ 157.40, 143.77, 141.82, 139.88, 139.65, 135.14, 134.42, 133.51, 133.35, 129.58, 128.37, 128.18, 123.04, 121.43, 121.35, 116.32, 115.26, 110.08, 46.76, 38.61, 31.48, 29.03; HRMS (ESI) m/z calcd for C₂₁H₁₉N₆ [M+H]⁺ 355.1671, found 355.1673.

### (4-(8,9,10,11-Tetrahydro-3H-pyrazolo[4,3-a]phenanthridin-7-yl)phenyl)boronic acid

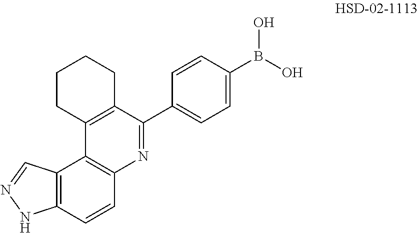 [SMILES: OB(O)c1ccc(-c2nc3ccc4[nH]ncc4c3c3c2CCCC3)cc1]

Method A: Off-white solid (151 mg, 44%). ¹H NMR (500 MHz, Methanol-d₄) δ 8.84 (s, 1H), 8.25 (dd, J=9.3, 3.1 Hz, 1H), 8.04 (dd, J=9.2, 3.2 Hz, 2H), 7.95 (s, 1H), 7.68 (d, J=7.5 Hz, 2H), 3.67-3.59 (m, 2H), 2.96-2.85 (m, 2H), 2.24-2.13 (m, 2H), 1.95-1.89 (m, 2H); ¹³C NMR (126 MHz, MeOD) δ 154.13, 151.37, 135.10, 134.26, 134.02, 133.60, 132.07, 128.01, 123.67, 119.63, 114.91, 30.85, 27.73, 21.31, 21.16; HRMS (ESI) m/z calcd for C₂₀H₁₉BN₃O₂ [M+H]⁺ 344.1570, found 344.1577.

### 4-(8,9,10,11-Tetrahydro-3H-pyrazolo[4,3-a]phenanthridin-7-yl)benzonitrile

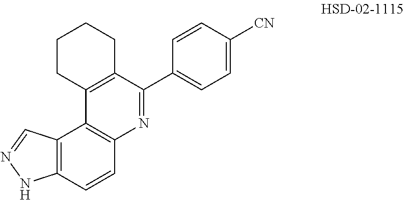 [SMILES: N#Cc1ccc(-c2nc3ccc4[nH]ncc4c3c3c2CCCC3)cc1]

Method A: Off-white solid (182 mg, 56%). ¹H NMR 500 MHz, DMSO-d₆) δ 8.78 (s, 1H), 8.20 (t, J=7.4 Hz, 2H), 8.10 (d, J=8.1 Hz, 2H), 7.94 (d, J=8.2 Hz, 2H), 3.48-3.44 (m, 2H), 2.73 (t, J=6.2 Hz, 2H), 2.07-1.97 (m, 2H), 1.84-1.72 (m, 2H); ¹³C NMR (126 MHz, DMSO) δ 150.26, 140.30, 138.64, 136.93, 135.01, 132.87, 131.23, 130.97, 123.45, 122.31, 119.99, 118.89, 115.01, 113.13, 30.63, 28.01, 21.85, 21.68; HRMS (ESI) m/z calcd for C₂₁H₁₇N₄ [M+H]⁺ 325.1453, found 325.1455.

### 4-(9-Amino-8,9,10,11-tetrahydro-3H-pyrazolo[4,3-a]phenanthridin-7-yl)phenol

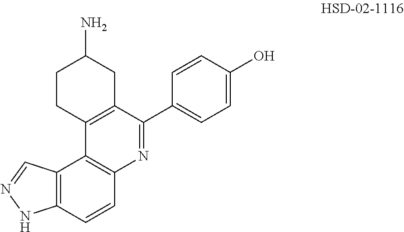 [SMILES: NC1CCc2c(c(-c3ccc(O)cc3)nc3ccc4[nH]ncc4c23)C1]

Method A: Off-white solid (205 mg, 62%). ¹H NMR (500 MHz, Methanol-d₄) δ 8.60-8.56 (m, 1H), 7.89 (d, J=9.2 Hz, 1H), 7.83 (dd, J=9.2, 0.9 Hz, 1H), 7.37 (d, J=8.6 Hz, 1H), 6.92 (d, J=8.6 Hz, 1H), 3.62-3.55 (m, 1H), 3.45-3.35 (m, 1H), 3.14-3.08 (m, 1H), 3.03-2.98 (m, 1H), 2.74-2.66 (m, 1H), 2.37-2.29 (m, 1H), 1.89-1.78 (m, 1H). ¹³C NMR (126 MHz, MeOD) δ 157.83, 157.59, 143.24, 142.36, 139.52, 134.57, 131.18, 130.07, 128.37, 128.06, 121.39, 116.01, 114.78, 114.55, 46.29, 37.35, 30.57, 28.84; HRMS (ESI) m/z calcd for C₂₀H₁₉N₄O [M+H]⁺ 331.1559, found 331.1561.

### (2-Fluoro-3-(8,9,10,11-tetrahydro-3H-pyrazolo[4,3-a]phenanthridin-7-yl)phenyl)boronic acid

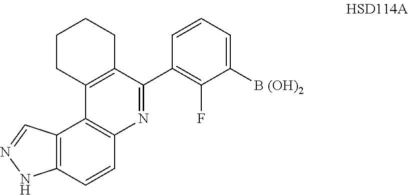

Method A: Off-white solid (163 mg, 45%). ¹H NMR (500 MHz, Methanol-d₄) δ 8.77 (s, 1H), 8.10 (s, 1H), 7.94 (d, J=7.3 Hz, 1H), 7.77-7.55 (m, 2H), 7.52-7.34 (m, 1H), 3.52 (s, 2H), 2.77 (s, 2H), 2.13 (s, 2H), 1.91 (s, 2H); ¹³C NMR (126 MHz, MeOD) δ 150.62, 147.92, 138.23, 136.61, 132.21, 132.13, 131.10, 124.78, 124.63, 123.70, 122.50, 122.02, 115.93, 115.76, 30.40, 26.98, 21.61, 21.26.

### 7-(3-(Trifluoromethyl)-1H-pyrazol-4-yl)-8,9,10,11-tetrahydro-3H-pyrazolo[4,3-a]phenanthridine

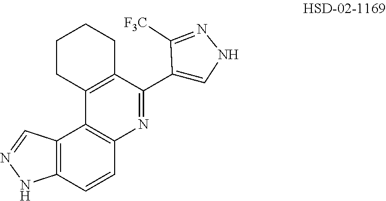 [SMILES: FC(F)(F)c1n[nH]cc1-c1nc2ccc3[nH]ncc3c2c2c1CCCC2]

Method A: Off-white solid (161 mg, 45%). ¹H NMR (500 MHz, DMSO-d₆) δ 8.55 (s, 1H), 8.22 (s, 1H), 7.85 (d, J=9.1 Hz, 1H), 7.77 (d, J=9.1 Hz, 1H), 3.26 (t, J=6.4 Hz, 2H), 2.68 (t, J=6.2 Hz, 2H), 1.98-1.86 (m, 2H), 1.80-1.69 (m, 2H); ¹³C NMR (126 MHz, DMSO) δ 148.52, 143.61, 141.99, 139.65, 139.37 (q, J=35.28 Hz), 135.14, 131.28, 130.36, 129.53, 123.56 (q, J 25=269.64 Hz), 121.99, 119.73, 116.21, 115.24, 29.48, 28.17, 22.51, 22.29; HRMS (ESI) m/z calcd for C₁₈H₁₅F₃N₅ [M+H]⁺ 358.1280, found 358.1286.

### 7-(1H-Benzo[d]imidazol-5-yl)-8,9,10,11-tetrahydro-3H-pyrazolo[4,3-a]phenanthridine

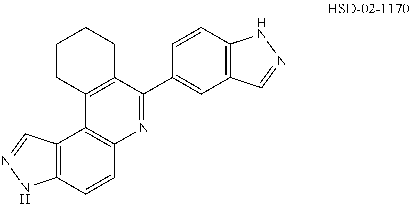 [SMILES: c1cc2[nH]ncc2cc1-c1nc2ccc3[nH]ncc3c2c2c1CCCC2]

Method A: Off-white solid (238 mg, 70%). ¹H NMR (500 MHz, DMSO-d₆) δ 8.58 (s, 1H), 8.28 (s, 1H), 7.85 (d, J=9.0, 0.8 Hz, 1H), 7.81 (d, J=9.1 Hz, 1H), 7.74 (s, 1H), 7.66 (d, J=8.3 Hz, 1H), 7.38 (dd, J=8.2, 1.6 Hz, 1H), 3.33 (t, J=6.6 Hz, 2H), 2.80 (t, J=6.1 Hz, 2H), 2.00 (td, J=9.0, 7.4, 4.6 Hz, 2H), 1.72 (ddt, J=9.2, 6.3, 3.8 Hz, 2H), ¹³C NMR (126 MHz, DMSO) δ 157.66, 143.73, 143.21, 142.24, 135.19, 129.71, 129.58, 123.77, 121.72, 116.34, 29.72, 29.29, 22.67, 22.62; HRMS (ESI) m/z calcd for C₂₁H₁₈N₅ [M+H]⁺ 340.1562, found 340.1570.

### 4-(1-Methyl-8,9,1011-tetrahydro-3H-pyrazolo[4,3-a]phenanthridin-7-yl)phenol

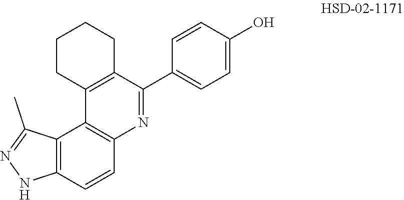 [SMILES: Cc1n[nH]c2ccc3nc(-c4ccc(O)cc4)c4c(c3c12)CCCC4]

Method A: Off-white solid (132 mg, 40%). ¹H NMR (500 MHz, DMSO-d₆) δ 10.37 (s, 1H), 8.24-8.03 (m, 2H), 7.56 (d, J=8.6 Hz, 2H), 7.04 (d, J=8.6 Hz, 2H), 3.62 (t, J=5.9 Hz, 2H), 2.94 (s, 3H), 2.81 (t, J=6.2 Hz, 2H), 1.93-1.73 (m, 4H); ¹³C NMR (126 MHz, DMSO) δ 163.50, 160.27, 153.31, 150.55, 135.81, 135.11, 131.86, 131.49, 125.28, 122.45, 119.96, 116.00, 112.81, 32.79, 27.62, 21.69, 21.32; HRMS (ESI) m/z calcd for C₂₁, H₂₀N₃O [M+H]⁺ 330.1606, found 330.1606.

### Methyl 7-(4-hydroxyphenyl)-8,9,10,11-tetrahydro-3H-pyrrolo[3,2-a]phenanthridine-2-carboxylate

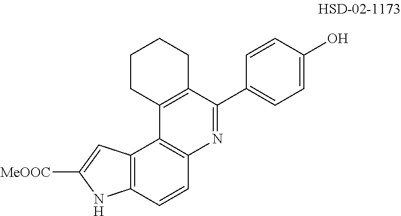 [SMILES: COC(=O)c1cc2c(ccc3nc(-c4ccc(O)cc4)c4c(c32)CCCC4)[nH]1]

Method A: Yellow solid (146 mg, 39%). ¹H NMR (500 MHz, DMSO-d₆) δ 9.79 (s, 1H), 7.83 (t, J=7.1 Hz, 2H), 7.71 (d, J=2.3 Hz, 1H), 7.42 (dd, J=8.6, 2.3 Hz, 2H), 6.90 (dd, J=8.4, 2.2 Hz, 2H), 3.35-3.32 (m, 8H), 2.77 (t, J=6.1 Hz, 2H), 2.02-1.92 (m, 2H), 1.78-1.65 (m, 2H); ¹³C NMR (126 MHz, DMSO) δ 161.47, 158.28, 155.16, 135.67, 131.12, 129.38, 126.63, 122.76, 119.77, 118.05, 115.31, 112.07, 30.25, 29.02, 22.48, 22.24; HRMS (ESI) m/z calcd for C₂₃H₂₃N₂O₃ [M+H]⁺ 375.1709, found 375.1711.

### 7-(4-Fluorophenyl)-8,9,10,11-tetrahydro-3H-pyrazolo[4,3-a]phenanthridine

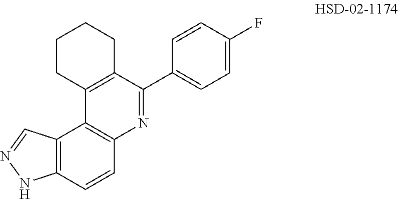 [SMILES: Fc1ccc(-c2nc3ccc4[nH]ncc4c3c3c2CCCC3)cc1]

Method A: Off-white solid (190 mg, 59%). ¹H NMR (500 MHz, DMSO-d₆) δ 8.80 (s, 1H), 8.24 (d, J=9.1 Hz, 1H), 8.20 (d, J=9.2 Hz, 1H), 7.85-7.77 (m, 2H), 7.52-7.44 (m, 2H), 3.47 (t, J=6.4 Hz, 2H), 2.76 (t, J=6.2 Hz, 2H), 2.07-1.98 (m, 2H), 1.83-1.71 (m, 2H); ¹³C NMR (126 MHz, DMSO) δ 164.66, 162.69, 150.54, 132.78, 132.71, 131.76, 129.72, 123.29, 121.12, 116.26, 116.08, 114.93, 111.46, 101.25, 30.79, 28.10, 21.80, 21.67; HRMS (ESI) m/z calcd for C₂₀H₂₁FN₃ [M+H]⁺ 322.1720, found 322.1724.

### 7-(4-Fluorophenyl)-8,9,10,11-tetrahydro-3H-pyrrolo[3,2-a]phenanthridine

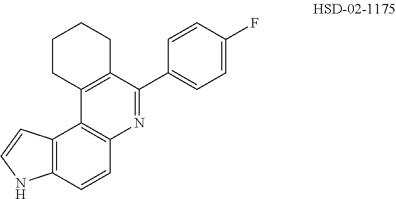 [SMILES: Fc1ccc(-c2nc3ccc4[nH]ccc4c3c3c2CCCC3)cc1]

Method A: Off-white solid (144 mg, 45%). ¹H NMR (500 MHz, DMSO-d₆) δ 11.76 (s, 1H), 7.78 (dd, J=8.9, 0.8 Hz, 1H), 7.63 (d, J=8.8 Hz, 1H), 7.61-7.54 (m, 2H), 7.49 (t, J=2.8 Hz, 1H), 7.32-7.24 (m, 2H), 7.12 (ddd, J=3.1, 2.0, 0.9 Hz, 1H), 3.38 (t, J=6.5 Hz, 2H), 2.74 (t, J=6.1 Hz, 2H), 2.01-1.92 (m, 2H), 1.72 (ddd, J=9.0, 7.1, 4.1 Hz, 2H); ¹³C NMR (126 MHz, DMSO) δ 163.08, 161.14, 154.88, 143.35, 142.44, 138.19, 133.25, 131.61, 131.54, 127.45, 124.06, 123.98, 122.53, 120.32, 116.56, 115.24, 115.07, 106.29, 29.96, 29.06, 22.84, 22.53; HRMS (ESI) m/z calcd for C₂₁H₂₂FN₂ [M+H]⁺ 321.1767, found 321.1768.

### 7-Phenyl-8,9,10,11-tetrahydro-3H-pyrrolo[3,2-a]phenanthridine

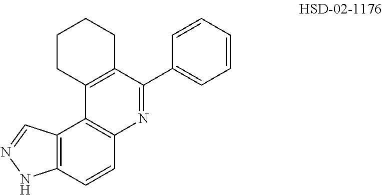 [SMILES: c1ccc(-c2nc3ccc4[nH]ncc4c3c3c2CCCC3)cc1]

Method A: Off-white solid (235 mg, 78%). ¹H NMR (500 MHz, DMSO-d₆) δ 8.56 (s, 1H), 7.84 (dd, J=9.1, 0.9 Hz, 1H), 7.80 (d, J=9.1 Hz, 1H), 7.54-7.51 (m, 2H), 7.48-7.44 (m, 2H), 7.44-7.39 (m, 1H), 3.29 (t, J=6.5 Hz, 2H), 2.74 (t, J=6.1 Hz, 2H), 2.02-1.91 (m, 2H), 1.77-1.64 (m, 2H); ¹³C NMR (126 MHz, DMSO) δ 156.89, 143.73, 142.34, 141.33, 139.70, 135.12, 129.62, 129.45, 129.22, 128.39, 128.15, 121.89, 116.26, 115.24, 29.64, 28.95, 22.59, 22.49; HRMS (ESI) m/z calcd for C₂₀H₁₈N₃ [M+H]⁺ 300.1501, found 300.1501.

### 7-Phenyl-8,9,10,11-tetrahydro-3H-pyrrolo[3,2-a]phenanthridine

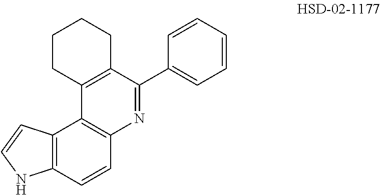 [SMILES: c1ccc(-c2nc3ccc4[nH]ccc4c3c3c2CCCC3)cc1]

Method A: Yellow solid (180 ng, 60%). ¹H NMR (500 MHz, Methanol-d₄) δ 8.19 (d, J=8.9 Hz, 1H), 7.84 (d, J=8.9 Hz, 1H), 7.72 (d, J=3.2 Hz, 1H), 7.69 (s, 5H), 7.42 (d, J=3.1 Hz, 1H), 3.71 (t, J=6.5 Hz, 2H), 2.87 (t, J=6.3 Hz, 2H), 2.16 (p, J=6.3 Hz, 3H), 1.92 (p, J=6.1 Hz, 2H), ¹³C NMR (126 MHz, MeOD) δ 154.99, 150.08, 133.99, 133.82, 132.55, 130.58, 129.45, 128.93, 128.81, 126.02, 123.68, 120.84, 120.09, 112.92, 106.45, 31.23, 27.74, 21.57, 21.19; HRMS (ESI) m/z calcd for C₂₁H₂₁N₂ [M+H]⁺ 301.1705, found 301.1713.

### 7-(4-Hydroxyphenyl)-8,9,10,11-tetrahydro-3H-pyrrolo[3,2-a]phenanthridine-2-carboxylic acid

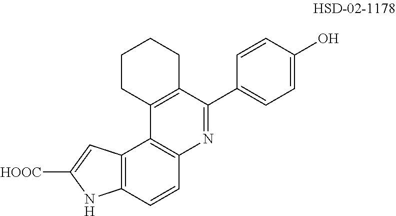 [SMILES: O=C(O)c1cc2c(ccc3nc(-c4ccc(O)cc4)c4c(c32)CCCC4)[nH]1]

Method A: Yellow solid (220 mg, 61%). ¹H NMR (500 MHz, DMSO-d₆) δ 10.44 (s, 1H), 8.29-7.84 (m, 2H), 7.77-7.44 (m, 3H), 7.06 (d, J=8.2 Hz, 2H), 3.56-3.32 (m, 2H), 2.94-2.62 (m, 2H), 2.14-1.89 (m, 2H), 1.75-1.55 (m, 2H); ¹³C NMR (126 MHz, DMSO) δ 162.48, 160.31, 153.04, 150.98, 135.29, 131.99, 130.84, 129.33, 123.31, 122.54, 121.26, 121.09, 119.04, 117.85, 115.91, 111.63, 31.11, 28.30, 21.81, 21.55; HRMS (ESI) m/z calcd for C₂₂H₂₁N₂O₃ [M+H]⁺ 361.1552, found 361.1552.

### 7-(2-Bromo-4-fluorophenyl)-8,9,10,11-tetrahydro-3H-pyrazolo[4,3-a]phenanthridine

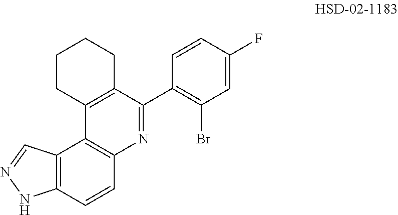 [SMILES: Fc1ccc(-c2nc3ccc4[nH]ncc4c3c3c2CCCC3)c(Br)c1]

Method A: Off-white solid (163 mg, 41%). ¹H NMR (500 MHz, Methanol-d₄) δ 8.56 (s, 1H), 7.84 (s, 2H), 7.55 (dd, J=8.5, 2.6 Hz, 1H), 7.42 (dd, J=8.5, 5.9 Hz, 1H), 7.29 (td, J=8.4, 2.6 Hz, 1H), 3.36-3.29 (m, 1H), 2.66-2.44 (m, 2H), 2.06-1.98 (m, 2H), 1.89-1.79 (m, 2H); ¹³C NMR (126 MHz, MeOD) δ 163.31 (J=250.74 Hz), 155.51, 143.71, 142.87, 137.54, 135.92, 131.53, 131.46, 130.07, 128.21, 122.77, 122.69, 122.62, 119.51, 119.32, 114.72, 114.55, 29.42, 27.22, 22.02, 21.75; HRMS (ESI) m/z calcd for C₂₀H₁₈BrFN₃ [M+H]⁺ 398.0668, found 398.0673.

### 7-(4-Ethynylphenyl)-8,9,10,11-tetrahydro-3H-pyrazolo[4,3-a]phenanthridine

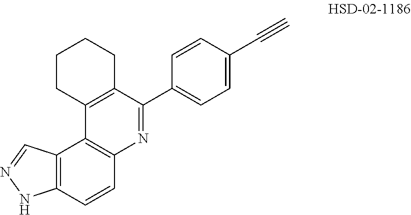 [SMILES: C#Cc1ccc(-c2nc3ccc4[nH]ncc4c3c3c2CCCC3)cc1]

Method A: Off-white solid (160 mg, 49%). ¹H NMR (500 MHz, DMSO-d₆) δ 8.54 (d, J=1.0 Hz, 1H), 7.88-7.84 (m, 1H), 7.79 (d, J=9.1 Hz, 1H) 7.64 (t, J=1.7 Hz, 1H), 7.58 (dt, J=7.6, 1.5 Hz, 1H), 7.54 (dt, J=7.7, 1.4 Hz, 1H), 7.47 (t, J=7.7 Hz, 1H), 4.21 (s, 1H), 3.25 (t, J=6.6 Hz, 2H), 2.73 (t, J=6.2 Hz, 2H), 1.99-1.92 (m, 2H), 1.70 (tdd, J=9.0, 5.6, 2.8 Hz, 2H); ¹³C NMR (126 MHz, DMSO) δ 155.63, 143.76, 142.49, 141.73, 139.83, 135.09, 132.65, 131.45, 130.16, 129.47, 129.13, 128.86, 122.07, 121.97, 116.19, 115.47, 83.85, 81.41 29.63, 28.83, 22.52, 22.45; HRMS (ESI) m/z calcd for C₂₂H₂₀N₃ [M+H]⁺ 326.1657, found 326.1659.

### 7-(4-Chlorophenyl)-8,9,10,11-tetrahydro-3H-naphtho[1,2-e]indazole

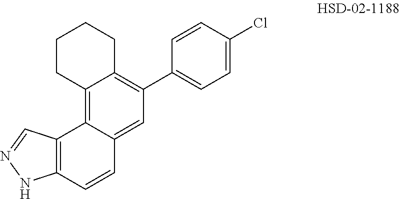 [SMILES: Clc1ccc(-c2cc3ccc4[nH]ncc4c3c3c2CCCC3)cc1]

Method A: Off-white solid (171 mg, 51%). ¹H NMR (500 MHz, DMSO-d₆) δ 8.54 (s, 1H), 7.85 (d, J=9.1 Hz, 1H), 7.81 (d, J=9.1 Hz, 1H), 7.61-7.55 (m, 2H), 7.55-7.47 (m, 2H), 3.30 (t, J=6.5 Hz, 2H), 2.74 (t, J=6.1 Hz, 2H), 2.01-1.91 (m, 2H), 1.76-1.66 (m, 2H); ¹³C NMR (126 MHz, DMSO) δ 155.62, 143.60, 143.11, 142.56, 140.08, 138.71, 136.38, 134.90, 133.05, 131.42, 129.61, 129.49, 129.30, 128.62, 128.43, 121.95, 116.41, 114.51, 29.67, 28.86, 22.54, 22.47; HRMS (ESI) m/z calcd for C₂₀H₁₉ClN₃ [M+H]⁺ 336.1268, found 336.1271.

### 7-(4-Chlorophenyl)-8,9,10,11-tetrahydro-3H-pyrrolo[3,2-a]phenanthridine

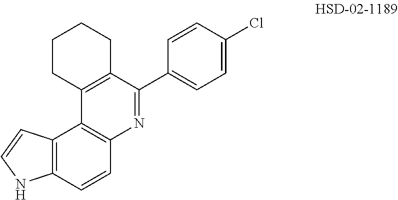 [SMILES: Clc1ccc(-c2nc3ccc4[nH]ccc4c3c3c2CCCC3)cc1]

Method A: Off-white solid (180 mg, 54%). ¹H NMR (500 MHz, Methanol-d₄) δ 7.75 (d, J=45.8 Hz, 2H), 7.46 (d, J=32.4 Hz, 5H), 7.20 (s, 1H), 3.50 (s, 2H), 2.74 (s, 2H), 2.06 (s, 2H), 1.83 (s, 2H); ¹³C NMR (126 MHz, MeOD) δ 154.78, 143.91, 142.58, 139.60, 133.67, 133.29, 130.38, 127.99, 127.27, 122.84, 121.93, 120.21, 116.07, 105.77, 29.93, 28.51, 22.42, 22.09; HRMS (ESI) m/z calcd for C₂₁H₂₀ClN₂ [M+H]⁺ 335.1315, found 335.1322.

### 7-(3-Fluorophenyl)-8,9,10,11-tetrahydro-3H-pyrazolo[4,3-a]phenanthridine

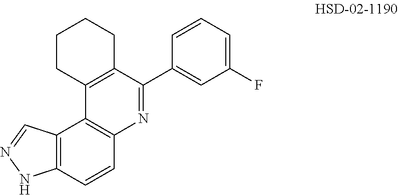 [SMILES: Fc1cccc(-c2nc3ccc4[nH]ncc4c3c3c2CCCC3)c1]

Method A: Off-white solid (195 mg, 61%). ¹H NMR (500 MHz, DMSO-d₆) δ 8.56 (s, 1H), 7.85 (dd, J=9.1, 0.9 Hz, 1H), 7.79 (d, J=9.1 Hz, 1H), 7.50 (td, J=8.0, 6.1 Hz, 1H), 7.42-7.36 (m, 2H), 7.30-7.22 (m, 1H), 3.27 (t, J=6.5 Hz, 2H), 2.75 (t, J=6.1 Hz, 2H), 2.01-1.92 (m, 2H), 1.78-1.66 (m, 2H); ¹³C NMR (126 MHz, DMSO) δ 163.27, 161.33, 155.43, 143.69, 143.64, 142.56, 130.42, 130.36, 129.58, 129.19, 125.72, 122.10, 116.43, 116.26, 116.19, 115.11, 114.94, 29.63, 28.75, 22.52, 22.43; HRMS (ESI) m/z calcd for C₂₀H₁₉FN₃ [M+H]⁺ 320.1563, found 320.1569.

### 7-(3-Fluorophenyl)-8,9,10,11-tetrahydro-3H-pyrrolo[3,2-a]phenanthridine

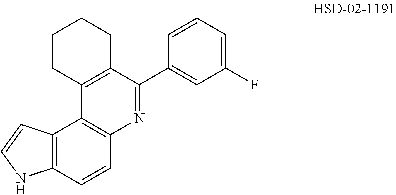 [SMILES: Fc1cccc(-c2nc3ccc4[nH]ccc4c3c3c2CCCC3)c1]

Method A: Off-white solid (160 mg, 50%). ¹H NMR (500 MHz, Methanol-d₄) δ 7.78 (ddd, J=8.9, 2.1, 0.8 Hz, 1H), 7.69 (dd, J=8.9, 3.8 Hz, 1H), 7.55-7.42 (m, 2H), 7.41 (dd, J=3.1, 1.2 Hz, 1H), 7.29 (dt, J=7.6, 1.2 Hz, 1H), 7.26-7.16 (m, 1H), 7.17 (dd, J=3.2, 1.0 Hz, 1H), 3.50-3.41 (m, 2H), 2.71 (t, J=6.3 Hz, 2H), 2.07-1.96 (m, 2H), 1.82-1.72 (m, 2H); ¹³C NMR (126 MHz, MeOD) δ 163.57 (J=245.70 Hz), 154.58, 143.91, 143.32, 142.54, 133.29, 130.38, 129.74, 129.68, 127.97, 127.14, 124.73, 122.96, 122.84, 121.97, 120.21, 116.09, 115.71, 115.54, 114.41, 114.24, 105.80, 29.89, 28.41, 22.39, 22.03; HRMS (ESI) m/z calcd for C₂₁H₂₀FN₂ [M+H]⁺ 319.1611, found 319.1618.

### 4-(8,9,10,11-Tetrahydro-3H-8,11-methanopyrazolo[4,3-a]phenanthridin-7-yl)phenol 1193

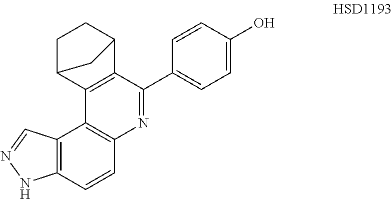 [SMILES: Oc1ccc(-c2nc3ccc4[nH]ncc4c3c3c2C2CCC3C2)cc1]

Method C: Off white solid (82 mg, 25%). ¹H NMR (500 MHz, Methanol-d₄) δ 8.64 (s, 1H), 7.97-7.90 (m, 1H), 7.80 (d, J=9.2 Hz, 1H), 7.62 (d, J=8.4 Hz, 2H), 6.97 (d, J=8.4 Hz, 2H), 4.34 (s, 1H), 3.76 (s, 1H), 2.33-2.18 (m, 2H), 1.93 (d, J=9.4 Hz, 1H), 1.77 (d, J=9.0 Hz, 1H), 1.44-1.31 (m, 2H), ¹³C NMR (126 MHz, MeOD) δ 157.96, 153.18, 150.46, 144.56, 139.98, 138.03, 134.29, 130.56, 130.03, 128.51, 116.16, 114.92, 113.81, 49.47, 43.72, 42.28, 26.38, 24.64. HRMS (ESI) m/z calcd for C₂₁H₁₈N₃O [M+H]⁺ 328.1450, found 328.1450.

### 4-(2-Methyl-8,9,10,11-tetrahydro-3H-pyrrolo[3,2-a]phenanthridin-7-yl)phenol

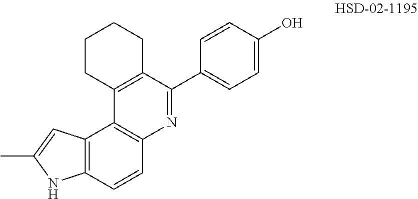 [SMILES: Cc1cc2c(ccc3nc(-c4ccc(O)cc4)c4c(c32)CCCC4)[nH]1]

Method A: Off-white solid (135 mg, 41%). ¹H NMR (500 MHz, Methanol-d₄) δ 7.66 (d, J=8.9 Hz, 1H), 7.61 (d, J=8.9 Hz, 1H), 7.34-7.29 (m, 2H), 6.93-6.88 (m, 2H), 6.85 (s, 1H), 3.42 (t, J=6.6 Hz, 2H), 2.73 (t, J=6.3 Hz, 2H), 2.53 (s, 3H), 2.06-1.97 (m, 2H), 1.83-1.73 (m, 2H); ³C NMR (126 MHz, MeOD) δ 157.11, 155.96, 143.48, 142.28, 133.63, 133.06, 132.20, 129.97, 127.27, 122.10, 121.13, 120.58, 115.11, 114.50, 103.96, 29.91, 28.69, 22.53, 22.23, 12.07; HRMS (ESI) m/z calcd for C₂₂H₂₃N₂O [M+H]⁺ 331.1810, found 331.1813.

### 6-(8,9,10,11-Tetrahydro-3H-pyrazolo[4,3-a]phenanthridin-7-yl)pyridin-3-ol

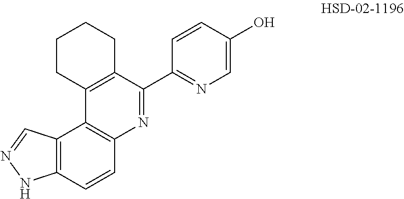 [SMILES: Oc1ccc(-c2nc3ccc4[nH]ncc4c3c3c2CCCC3)nc1]

Method A: Off-white solid (124 mg, 39%). ¹H NMR (500 MHz, Methanol-d₄) δ 8.57 (s, 1H), 8.22 (d, J=2.8 Hz, 1H), 7.90 (d, J=9.8 Hz, 1H), 7.83 (d, J=9.0 Hz, 1H), 7.55 (d, J=8.4 Hz, 1H), 7.41 (dd, J=8.5, 2.8 Hz, 1H), 3.38 (s, 2H), 2.82 (t, J=6.2 Hz, 2H), 2.12-2.04 (m, 2H), 1.89-1.80 (m, 2H); ¹³C NMR (126 MHz, MeOD) δ 154.85, 154.08, 149.15, 143.75, 142.80, 138.71, 136.11, 135.86, 130.19, 128.51, 125.23, 123.37, 122.48, 116.10, 113.81, 29.62, 27.62, 22.09, 21.91; HRMS (ESI) m/z calcd for C₁₉H₁₇N₄O [M+H]⁺ 317.1402, found 317.1397.

### 7-(6-Fluoropyridin-3-yl)-8,9,10,11-tetrahydro-3H-pyrrolo[3,2-a]phenanthridine

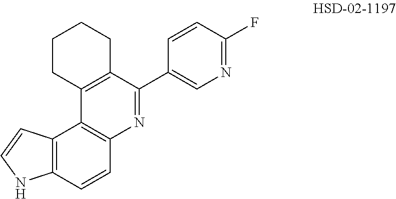 [SMILES: Fc1ccc(-c2nc3ccc4[nH]ccc4c3c3c2CCCC3)cn1]

Method A: Off-white solid (201 mg, 61%). ¹H NMR (500 MHz, Methanol-d₄) δ 8.31 (d, J=2.4 Hz, 1H), 8.04 (td, J=8.0, 2.5 Hz, 1H), 7.78 (d, J=8.9 Hz, 1H), 7.68 (d, J=8.9 Hz, 1H), 7.40 (d, J=3.1 Hz, 1H), 7.17 (dd, J=8.4, 2.3 Hz, 1H), 7.12 (d, J=3.1 Hz, 1H), 3.36 (t, J=6.5 Hz, 2H), 2.66 (t, J=6.2 Hz, 2H), 1.95 (ddt, J=12.5, 9.1, 4.6 Hz, 2H), 1.79-1.71 (m, 2H), ¹³C NMR (126 MHz, MeOD) δ 164.07, 162.16, 151.30, 147.35, 147.24, 144.14, 142.80, 142.65, 142.58, 134.95, 134.91, 133.32, 127.42, 123.11, 122.96, 122.05, 120.13, 116.37, 116.32, 108.83, 108.54, 105.95, 105.90, 29.82, 28.39, 22.24, 21.95; HRMS (ESI) m/z calcd for C₂₀H₁₉FN₃ [M+H]⁺ 320.1563, found 320.1566.

### 7-(Pyridin-3-yl)-8,9,10,11-tetrahydro-3H-pyrazolo[4,3-a]phenanthridine

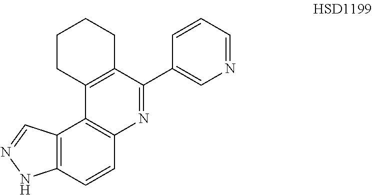 [SMILES: c1cncc(-c2nc3ccc4[nH]ncc4c3c3c2CCCC3)c1]

Method A: Off-white solid (135 mg, 45%). ¹H NMR (500 MHz, Methanol-d₄) δ 9.06 (s, 2H), 8.73 (s, 1H), 8.42 (d, J=7.3 Hz, 1H), 8.11 (d, J=8.4 Hz, 1H), 7.96 (d, J=8.8 Hz, 2H), 3.55-3.46 (m, 2H), 2.95-2.81 (m, 2H), 2.20-2.08 (m, 2H), 1.99-1.84 (m, 2H); ¹³C NMR (126 MHz, MeOD) δ 150.86, 148.85, 148.27, 147.36, 140.15, 138.36, 131.44, 123.81, 122.94, 119.15, 115.51, 30.47, 27.89, 21.51, 21.42; HRMS (ESI) m/z calcd for C₁₉H₁₇N₄ [M+H]⁺ 301.1453, found 301.1453.

### 7-(2-Bromophenyl)-8,9,10,11-tetrahydro-3H-pyrrolo[3,2-a]phenanthridine

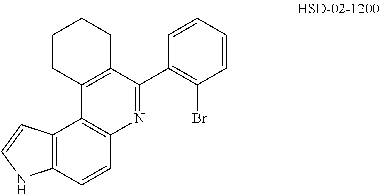 [SMILES: Brc1ccccc1-c1nc2ccc3[nH]ccc3c2c2c1CCCC2]

Method A: Off-white solid (223 mg, 59%). ¹H NMR (500 MHz, Methanol-d₄) δ 7.81 (dd, J=8.9, 0.8 Hz, 1H), 7.73 (dd, J=8.5, 1.2 Hz, 1H), 7.69 (d, J=8.9 Hz, 1H), 7.51 (td, J=7.5, 1.2 Hz, 1H), 7.44 (d, J=3.1 Hz, 1H), 7.40-7.36 (m, 2H), 7.21 (dd, J=3.2, 0.9 Hz, 1H), 3.50 (td, J=6.4, 1.4 Hz, 2H), 2.57 (ddt, J=68.3, 17.0, 6.3 Hz, 2H), 2.08-1.98 (m, 2H), 1.92-1.78 (m, 2H). ¹³C NMR (126 MHz, MeOD) δ 155.07, 143.75, 142.37, 141.72, 133.32, 132.31, 130.23, 130.19, 129.54, 129.50, 127.66, 127.44, 123.14, 122.89, 122.52, 121.82, 120.27, 116.01, 105.69, 29.80, 27.36, 22.39, 21.88; H₁RMS (ESI) m/z, calcd for C₂₁H₂₀BrN₂ [M+H]⁺ 379.0810, found 379.0817.

### 3-Fluoro-4-(8,9,10,11-tetrahydro-3H-pyrrolo[3,2-a]phenanthridin-7-yl)phenol

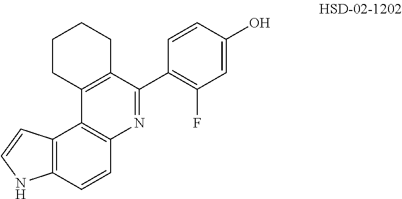 [SMILES: Oc1ccc(-c2nc3ccc4[nH]ccc4c3c3c2CCCC3)c(F)c1]

Method A: Off-white solid (184 mg, 55%). ¹H NMR (500 MHz, Methanol-d₄) δ 11.65 (s, 1H), 9.02 (d, J=8.9 Hz, 1H), 8.80 (d, J=9.0 Hz, 1H), 8.60 (s, 1H), 8.34 (t, J=8.5 Hz, 1H), 8.13 (s, 1H), 7.72 (d, J=8.1 Hz, 2H), 4.37 (s, 2H), 3.52 (d, J=6.3 Hz, 2H), 2.93-2.78 (m, 2H), 2.61 (s, 2H), ¹³C NMR (126 MHz, MeOD) δ 163.03, 162.14, 160.18, 134.61, 133.62, 131.43, 127.83, 124.37, 122.33, 120.67, 113.54, 107.72, 104.37, 104.18, 31.98, 28.02, 22.84, 22.19; HRMS (ESI) m/z calcd for C₂₁H₂₀FN₂O [M+H]⁺ 335.1560, found 335.1566.

### 2-Fluoro-4-(2-methyl-8,9,10,11-tetrahydro-3H-pyrrolo[3,2-a]phenanthridin-7-yl)phenol

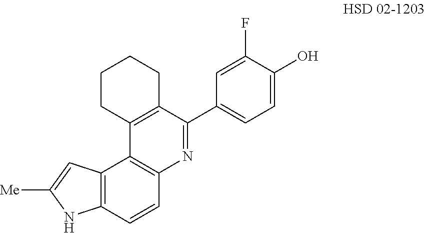 [SMILES: Cc1cc2c(ccc3nc(-c4ccc(O)c(F)c4)c4c(c32)CCCC4)[nH]1]

Method A: Off-white solid (164 mg, 47%). ¹H NMR (500 MHz, Methanol-d₄) δ 7.66 (d, J=8.9 Hz, 1H), 7.61 (d, J=8.9 Hz, 1H), 7.20 (dd, J=11.8, 2.0 Hz, 1H), 7.13-7.08 (m, 1H), 7.05-7.00 (m, 1H), 6.84 (s, 1H), 3.39 (t, J=6.5 Hz, 2H), 2.72 (t, J=6.2 Hz, 2H), 2.52 (s, 3H), 2.04-1.94 (m, 2H), 1.82-1.70 (m, 2H); ¹³C NMR (126 MHz, MeOD) δ 154.57, 152.03, 150.11, 144.90, 143.84, 142.21, 133.78, 133.12, 132.62, 127.08, 125.12, 122.26, 121.10, 120.50, 117.06, 116.55, 116.40, 115.35, 104.03, 29.91, 28.58, 22.44, 22.15, 12.08; HRMS (ESI) m/z calcd for C₂₂H₂₂FN₂O [M+H]⁺ 349.1716, found 349.1720.

### 4-(8,9,10,11-Tetrahydro-3H-pyrazolo[4,3-a]phenanthridin-7-yl)benzimidamide hydrochloride

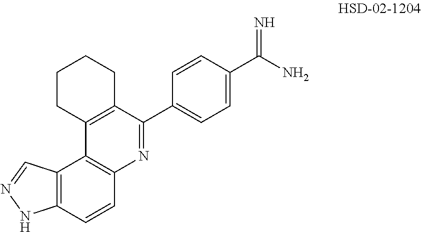 [SMILES: N=C(N)c1ccc(-c2nc3ccc4[nH]ncc4c3c3c2CCCC3)cc1]

Method A: Off-white solid (172 mg, 50%). ¹H NMR (500 MHz, DMSO-d₆) δ 9.69 (s, 2H), 9.47 (s, 2H), 8.71 (s, 1H), 8.16-8.06 (m, 4H), 7.94 (d, J=8.0 Hz, 2H), 3.42-3.33 (m, 2H), 2.76 (t, J=6.2 Hz, 2H), 2.07-1.97 (m, 2H), 1.81-1.65 (m, 2H); ¹³C NMR (126 MHz, DMSO) δ 165.63, 151.52, 140.26, 134.84, 130.64, 130.34, 129.16, 128.63, 123.10, 115.22, 30.40, 28.25, 21.97, 21.83; HRMS (ESI) m/z calcd for C₂₁H₂₂N₅ [M+H]⁺ 344.1875. found 344.1879.

### N-hydroxy-4-(8,9,10,11-tetrahydro-3H-pyrazolo[4,3-a]phenanthridin-7-yl)benzimidamide

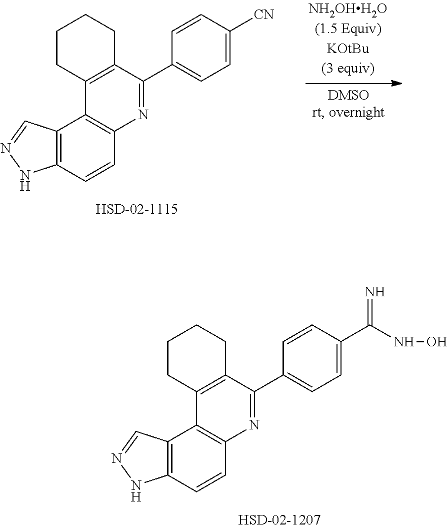 [SMILES: N#Cc1ccc(-c2nc3ccc4[nH]ncc4c3c3c2CCCC3)cc1 ; N=C(NO)c1ccc(-c2nc3ccc4[nH]ncc4c3c3c2CCCC3)cc1]

In a solution of NH₂OH·H₂O (1.5 equiv) in DMSO (3 mL), KOtBut (3 equiv) was added slowly at 0° C. and the suspension was stirred for 30 min. After this 4-(8,9,10,11-Tetrahydro-3H-pyrazolo[4,3-a]phenanthridin-7-yl)benzonitrile(0.5 mmol) was added to the mixture and reaction was continued for 4 h at room temperature. After completion of reaction cold water was added to the reaction mixture and the resulting precipitate was filtered and washed with water and dried. Solid was recrystallized with ethanol to get the desired product.

Off-white solid (162 mg, 90%). ¹H NMR (500 MHz, DMSO-d₆) δ 9.71 (s, 1H), 8.56 (s, 1H), 7.88-7.82 (m, 2H), 7.79-7.76 (m, 2H), 7.59-7.54 (m, 2H), 5.89 (s, 2H), 2.79 (t, J=6.1 Hz, 2H), 2.00 (ddt, J=9.3, 6.5, 3.1 Hz, 2H), 1.74 (tt, J=8.6, 5.4 Hz, 2H); ¹³C NMR (126 MHz, DMSO) δ 156.48, 151.11, 145.66, 143.64, 142.45, 141.74, 138.70, 136.39, 133.15, 129.67, 129.32, 125.43, 121.88, 116.46, 114.44, 29.69, 28.96, 22.60, 22.52; HRMS (ESI) m/z calcd for C₂₁H₂₂N₅ [M+H]⁺ 360.1824, found 360.1827.

### N-(4-(8,9,10,11-Tetrahydro-3H-pyrazolo[4,3-a]phenanthridin-7-yl)phenyl)acetamide

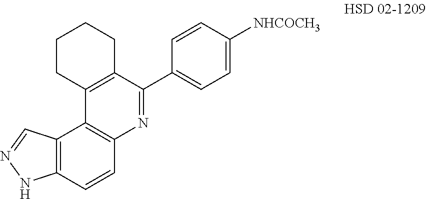

Method A: Off-white solid (201 mg, 51%). ¹H NMR (500 MHz, DMSO-d₆) δ 10.08 (s, 1H), 8.50 (s, 1H), 7.81 (s, 2H), 7.70 (d, J=8.4 Hz, 2H), 7.49 (d, J=8.5 Hz, 2H), 3.24 (t, J=6.0 Hz, 2H), 2.75 (t, J=6.1 Hz, 2H), 2.08 (s, 3H), 1.96-1.89 (m, 2H), 1.67 (tt, J=8.3, 5.4 Hz, 2H); ¹³C NMR (126 MHz, DMSO) δ 168.87, 156.52, 143.62, 142.22, 139.37, 138.62, 136.27, 135.92, 129.99, 129.62, 129.35, 121.66, 118.79, 116.48, 114.22, 29.63, 29.05, 24.54, 22.59, 22.54; HRMS (ESI) m/z calcd for C₂₂H₂₃N₄O [M+H]⁺ 359.1872, found 359.1875.

### 4-(3,8-Dihydro-2H-furo[3,2-c]pyrrolo[3,2-f]quinolin-4-yl)phenol

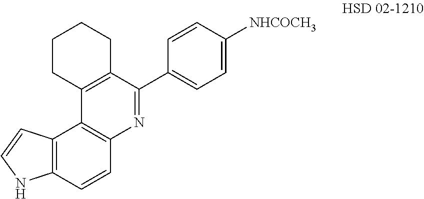

Method A: Off-white solid (171 mg, 48%). ¹H NMR (500 MHz, DMSO-d₆) δ 11.74 (t, J=2.4 Hz, 1H), 10.05 (s, 1H), 7.78 (d, J=8.8 Hz, 1H), 7.68 (d, J=8.3 Hz, 2H), 7.63 (d, J=8.8 Hz, 1H), 7.51-7.46 (m, 3H), 7.10 (d, J=2.8 Hz, 1H), 3.38-3.35 (m, 2H), 2.77 (t, J=6.2 Hz, 2H), 2.08 (s, 3H), 1.99-1.92 (m, 2H), 1.71 (ddt, J=12.3, 9.5, 4.4 Hz, 2H); ¹³C NMR (126 MHz, DMSO) δ 168.82, 155.57, 143.41, 142.25, 139.16, 136.45, 133.19, 129.95, 127.55, 124.10, 123.88, 122.36, 120.37, 118.76, 116.42, 106.27, 29.98, 29.18, 24.55, 22.90, 22.61; HRMS (ESI) m/z calcd for C₂₃H₂₄N₃O [M+H]⁺ 358.1919, found 358.1922.

### 4-(3,8-Dihydro-2H-furo[3,2-c]pyrrolo[3,2-f]quinolin-4-yl)phenol

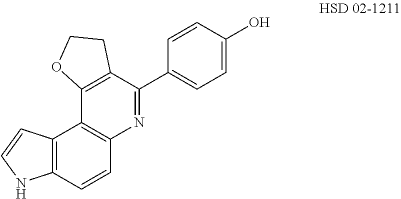 [SMILES: Oc1ccc(-c2nc3ccc4[nH]ccc4c3c3c2CCO3)cc1]

Method B: Off-white solid (76 mg, 25%). ¹H NMR (500 MHz, Methanol-d₄) δ 8.62 (s, 1H), 7.77 (d, J=9.0 Hz, 1H), 7.67 (d, J=9.0 Hz, 1H), 7.42-7.32 (m, 3H), 7.11 (d, J=3.0 Hz, 1H), 6.96-6.89 (m, 2H), 3.71 (t, J=7.0 Hz, 2H), 3.07 (t, J=7.1 Hz, 2H); ¹³C NMR (126 MHz, MeOD) δ 157.38, 156.71, 133.00, 132.35, 131.76, 130.10, 129.51, 123.71, 122.39, 121.62, 120.57, 116.48, 114.65, 106.36, 100.91, 61.71, 35.83; HRMS (ESI) m/z calcd for C₁₉H₁₇N₂O₂ [M+H]⁺ 305.1290, found 305.1299.

### 2-Fluoro-4-(1-meth 1-8,9,10,11-tetrahydro-3H-pyrazolo[4,3-a]phenanthridin-7-yl)phenol

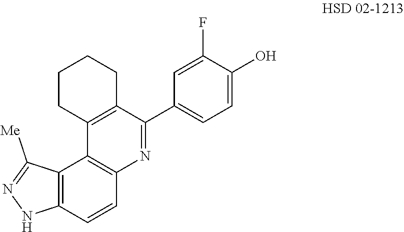 [SMILES: Cc1n[nH]c2ccc3nc(-c4ccc(O)c(F)c4)c4c(c3c12)CCCC4]

Method A: Off-white solid (157 mg, 45%). ¹H NMR (500 MHz, Methanol-d₄) δ 7.76 (s, 1H), 7.71 (d, J=8.6 Hz, 1H), 7.21 (d, J=11.7 Hz, 1H), 7.12 (d, J=8.4 Hz, 1H), 7.04 (t, J=8.5 Hz, 1H), 3.51-3.40 (m, 2H), 2.89 (s, 3H), 2.84-2.76 (m, 2H), 1.92-1.80 (m, 4H); C NMR (126 MHz, MeOD) δ 157.03, 155.85, 152.11, 150.19, 144.98, 143.53, 132.05, 128.98, 128.70, 125.04, 123.95, 117.20, 116.48, 116.32, 113.04, 31.86, 27.70, 22.03, 21.87; HRMS (ESI) m/z calcd for C₂₁H₂₁FN₃O [M+H]⁺ 350.1669, found 350.1677.

### 4-(8,9,10,11-Tetrahydro-3H-pyrrolo[3,2-a]phenanthridin-7-yl)benzonitrile

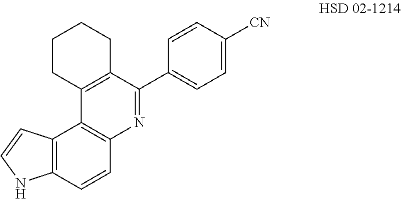 [SMILES: N#Cc1ccc(-c2nc3ccc4[nH]ccc4c3c3c2CCCC3)cc1]

Method A: Off-white solid (195 mg, 60%). ¹H NMR (500 MHz, DMSO-d₆) δ 11.80 (s, 1H), 7.92 (d, J=8.5 Hz, 1H), 7.81 (dd, J=8.9, 0.8 Hz, 1H), 7.75 (d, J=8.9 Hz, 2H), 7.64 (d, J=8.9 Hz, 1H), 7.51 (t, J=2.8 Hz, 1H), 7.15-7.11 (m, 1H), 3.38 (t, J=6.1 Hz, 2H), 2.73 (t, J=6.1 Hz, 2H), 2.00-1.93 (m, 3H), 1.76-1.68 (m, 2H); ¹³C NMR (126 MHz, DMSO) δ 154.08, 146.45, 143.42, 142.77, 133.40, 132.39, 130.61, 127.28, 124.14, 124.07, 122.82, 120.26, 119.41, 116.85, 110.74, 106.41, 29.95, 28.81, 22.76, 22.41; HRMS (ESI) m/z calcd for C₂₂H₂₀N₃[M+H]⁺ 326.1657, found 326.1662.

### Methyl 4-(8,9,10,11-tetrahydro-3H-pyrazolo[4,3-a]phenanthridin-7-yl)benzoate

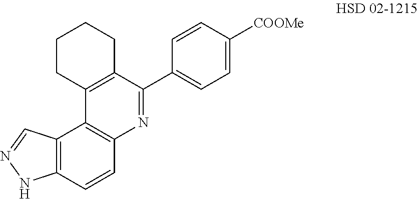 [SMILES: COC(=O)c1ccc(-c2nc3ccc4[nH]ncc4c3c3c2CCCC3)cc1]

Method A: Off-white solid (198 ng, 55%). ¹H NMR (500 MHz, DMSO-d₆) δ 8.69 (s, 1H), 8.14 (dd, J=10.7, 7.0 Hz, 4H), 7.87 (d, J=7.6 Hz, 2H), 3.90 (s, 3H), 3.35 (d, J=6.2 Hz, 2H), 2.73 (t, J=5.8 Hz, 2H), 2.06-1.96 (m, 2H), 1.75 (d, J=7.1 Hz, 2H); ¹³C NMR (126 MHz, DMSO) δ 166.53, 155.59, 145.92, 143.71, 142.57, 138.77, 136.40, 130.03, 129.92, 129.65, 129.55, 129.25, 129.09, 122.12, 116.33, 114.56, 52.63, 29.59, 28.75, 22.48, 22.38; HRMS (ESI) m/z calcd for C₂₂H₂₂N₃O₂ [M+H]⁺ 360.1712, found 360.1718.

### 4-(8,9,10,11-Tetrahydro-3H-pyrazolo[4,3-a]phenanthridin-7-yl)benzamide

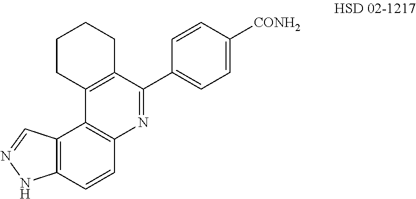 [SMILES: NC(=O)c1ccc(-c2nc3ccc4[nH]ncc4c3c3c2CCCC3)cc1]

Method A: Off-white solid (172 mg, 50%). ¹H NMR (500 MHz, DMSO-d₆) δ 8.60 (s, 1H), 8.05 (s, 1H), 7.97 (d, J=8.0 Hz, 2H), 7.86 (d, J=9.1 Hz, 1H), 7.81 (d, J=9.1 Hz, 1H), 7.63 (d, J=8.0 Hz, 2H), 7.41 (S, 1H), 3.39-3.34 (m, 2H), 2.77 (t, J=6.1 Hz, 2H), 2.04-1.98 (m, 2H), 1.79-1.69 (m, 2H); ¹³C NMR (126 MHz, DMSO) δ 168.13, 156.17, 144.02, 143.77, 142.55, 133.95, 129.63, 129.40, 129.29, 127.64, 122.07, 116.23, 29.68, 28.86, 22.58, 22.49; HRMS (ESI) m/z calcd for C₂₁H₂₁N₄O [M+H]⁺ 345.1715, found 345.1719.

### Methyl 4-(8,9,10,11-tetrahydro-3H-pyrrolo[3,2-a]phenanthridin-7-yl)benzoate

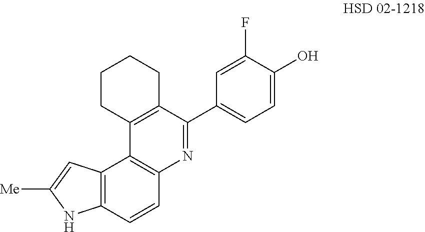 [SMILES: Cc1cc2c(ccc3nc(-c4ccc(O)c(F)c4)c4c(c32)CCCC4)[nH]1]

Method A: Off-white solid (105 mg, 30%). ¹H NMR (500 MHz, Methanol-d₄) δ 7.68 (dd, J=8.9, 0.8 Hz, 1H), 7.62 (d, J=8.9 Hz, 1H), 7.22 (dd, J=11.8, 2.0 Hz, 1H), 7.13 (ddd, J=8.2, 2.1, 0.9 Hz, 1H), 7.03 (dt, J=8.9, 8.2 Hz, 1H), 6.87 (s, 1H), 3.48-3.40 (m, 2H), 2.76 (t, J=6.2 Hz, 2H), 2.54 (s, 3H), 2.07-1.99 (m, 2H), 1.86-1.75 (m, 2H); ¹³C NMR (126 MHz, MeOD) δ 154.38, 152.03 (J=241.92 Hz), 144.97, 144.23, 141.91, 133.92, 133.16, 132.30, 127.16, 125.13, 122.28, 121.10, 120.18, 117.07, 116.57, 116.41, 115.50, 104.05, 29.98, 28.55, 22.44, 22.14, 12.07; HRMS (ESI) m/z calcd for C₂₂H₂₂FN₂O [M+H]⁺ 349.1716, found 349.1717.

### Methyl 4-(8,9,10,11-tetrahydro-3H-pyrrolo[3,2-a]phenanthridin-7-yl)benzoate

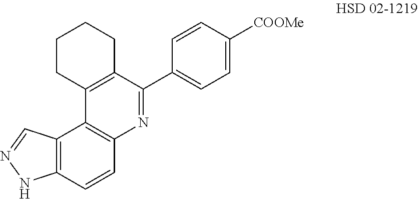 [SMILES: COC(=O)c1ccc(-c2nc3ccc4[nH]ncc4c3c3c2CCCC3)cc1]

Method A: Off-white solid (179 mg, 50%). ¹H NMR (500 MHz, DMSO-d₆) δ 12.38 (s, 1H), 8.13 (d, J=8.0 Hz, 2H), 8.08 (d, J=8.7 Hz, 1H), 7.92 (d, J=8.9 Hz, 1H), 7.82 (d, J=8.0 Hz, 2H), 7.69 (t, J=2.7 Hz, 1H), 7.25-7.22 (m, 1H), 3.90 (s, 3H), 3.48 (t, J=6.5 Hz, 2H), 2.72 (t, J=6.2 Hz, 2H), 2.06-1.94 (m, 2H), 1.85-1.67 (m, 3H); ¹³C NMR (126 MHz, DMSO) δ 166.31, 150.76, 140.46, 137.32, 133.66, 130.77, 130.49, 130.13, 129.56, 129.56, 129.45, 128.64, 126.02, 123.33, 119.96, 117.68, 106.71, 52.90, 30.82, 28.26, 22.22, 21.82; HRMS (ESI) m/z calcd for C₂₃H₂₃N₂O₂ [M+H]⁺ 359.1760, found 359.1767.

### N-Hydroxy-4-(8,9,10,11-tetrahydro-3H-pyrrolo[3,2-a]phenanthridin-7-yl)benzimidamide

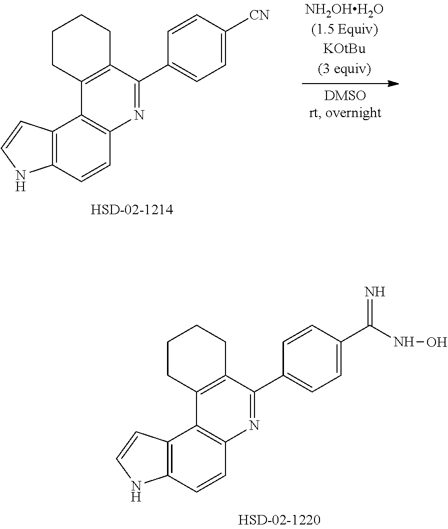 [SMILES: N#Cc1ccc(-c2nc3ccc4[nH]ccc4c3c3c2CCCC3)cc1 ; N=C(NO)c1ccc(-c2nc3ccc4[nH]ccc4c3c3c2CCCC3)cc1]

Synthesized by following general procedure for 1207. Off-white solid (163 mg, 91%); ¹H NMR (500 MHz, DMSO-d₆) δ 11.76 (d, J=2.3 Hz, 1H), 9.69 (s, 1H), 7.78 (t, J=8.9 Hz, 3H), 7.63 (d, J=8.8 Hz, 1H), 7.56-7.52 (m, 2H), 7.49 (t, J=2.8 Hz, 1H), 7.13 (d, J=2.5 Hz, 1H), 5.86 (s, 2H), 3.39 (t, J=6.5 Hz, 3H), 2.77 (t, J=6.2 Hz, 2H), 2.01-1.92 (m, 2H), 1.79-1.69 (m, 2H). ³C NMR (126 MHz, DMSO) δ 155.44, 151.17, 143.41, 142.38, 142.28, 133.25, 132.92, 129.31, 127.45, 125.38, 124.12, 123.98, 122.52, 120.34, 116.54, 106.31, 29.97, 29.08, 22.87, 22.54; HRMS (ESI) m/z calcd for C₂₂H₂₃N₄O [M+H]⁺ 359.1872, found 359.1872.

### 4-(8,9,10,11-Tetrahydro-3H-pyrrolo[3,2-a]phenanthridin-7-yl)benzamide

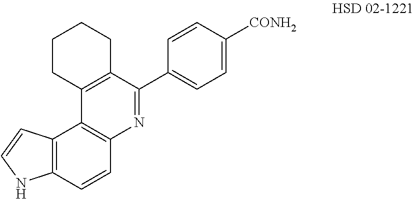 [SMILES: NC(=O)c1ccc(-c2nc3ccc4[nH]ccc4c3c3c2CCCC3)cc1]

Method A: Off-white solid (147 mg, 43%); ¹H NMR (500 MHz, DMSO-d₆) δ 11.77 (t, J=2.3 Hz, 1H), 8.05 (s, 1H), 7.97 (d, J=8.3 Hz, 2H), 7.79 (d, J=8.9 Hz, 1H), 7.64 (d, J=8.9 Hz, 1H), 7.61 (d, J=8.2 Hz, 2H), 7.50 (t, J=2.8 Hz, 1H), 7.41 (s, 1H), 7.13 (t, J=2.5 Hz, 1H), 3.39 (t, J=6.5 Hz, 2H), 2.75 (t, J=6.1 Hz, 2H), 1.98 (q, J=4.3, 3.0 Hz, 3H), 1.73 (ddt, J=9.4, 6.5, 2.8 Hz, 2H), ¹³C NMR (126 MHz, DMSO) δ 168.20, 155.18, 144.56, 143.40, 142.47, 133.73, 133.30, 129.39, 127.59, 127.40, 124.11, 124.02, 122.62, 120.33, 116.62, 106.34, 29.97, 28.98, 22.84, 22.50; HRMS (ESI) m/z calcd for C₂₂H₂₂N₃O [M+H]⁺ 344.1763, found 344.1769.

### 2,3-Difluoro-4-(8,9,10,11-tetrahydro-3H-pyrrolo[3,2-a]phenanthridin-7-yl)phenol

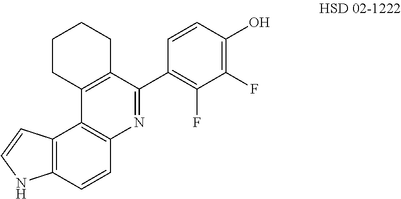 [SMILES: Oc1ccc(-c2nc3ccc4[nH]ccc4c3c3c2CCCC3)c(F)c1F]

Method A: Yellow solid (166 mg, 47%). ¹H NMR (500 MHz, DMSO-d₆) δ 11.40 (s, 1H), 8.23 (d, J=8.9 Hz, 1H), 8.05 (d, J=8.9 Hz, 1H), 7.80 (t, J=2.9 Hz, 1H), 7.41-7.34 (m, 1H), 7.31 (t, J=2.4 Hz, 1H), 7.15 (t, J=8.2 Hz, 1H), 3.55 (t, J=6.4 Hz, 2H), 2.72 (s, 2H), 2.13-1.96 (m, 2H), 1.81 (dd, J=7.8, 4.1 Hz, 2H), ¹³C NMR (126 MHz, DMSO) δ 149.88, 147.97, 147.88, 143.40, 141.12, 141.01, 139.18, 139.07, 134.54, 133.83, 130.60, 127.12, 126.30, 123.79, 121.80, 119.78, 113.94, 111.57, 106.98, 31.22, 27.17, 21.96, 21.33; HRMS (ESI) m/z calcd for C₂₁H₁₉F₂N₂O [M+H]⁺ 353.1465, found 353.1468.

### 2,3-Difluoro-4-(8,9,10,11-tetrahydro-3H-pyrazolo[4,3-a]phenanthridin-7-yl)phenol

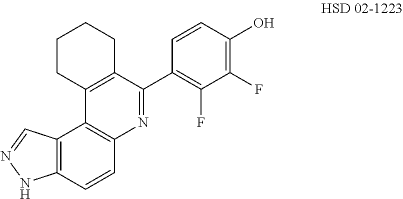 [SMILES: Oc1ccc(-c2nc3ccc4[nH]ncc4c3c3c2CCCC3)c(F)c1F]

Method A: Off-white solid (177 mg, 50%). ¹H NMR (500 MHz, DMSO-d₆) δ 10.56 (s, 1H), 8.56 (s, 1H), 7.92-7.76 (m, 2H), 7.05 (td, J=8.2, 2.0 Hz, 1H), 6.90 (td, J=8.3, 1.6 Hz, 1H), 3.32 (d, J=7.6 Hz, 2H), 2.62 (t, J=6.1 Hz, 2H), 1.96 (dt, J=11.1, 5.9 Hz, 2H), 1.75 (p, J=5.8 Hz, 2H); ¹³C NMR (126 MHz, DMSO) δ 151.48, 149.77, 147.83, 147.75, 147.03, 143.67, 142.40, 141.06, 140.95, 139.02, 138.74, 136.40, 130.47, 129.50, 125.40, 122.18, 120.81, 120.70, 116.39, 114.56, 113.32, 29.50, 27.48, 22.58, 22.19; HRMS (ESI) m/z calcd for C₂₀H₁₆F₂N₃O [M+H]⁺ 352.1261, found 352.1256.

### 2-Fluoro-5-(8,9,10,11-tetrahydro-3H-pyrazolo[4,3-a]phenanthridin-7-yl)benzonitrile

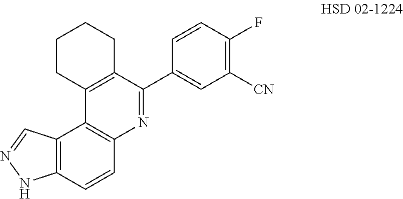 [SMILES: N#Cc1cc(-c2nc3ccc4[nH]ncc4c3c3c2CCCC3)ccc1F]

Method A: Off-white solid (169 mg, 49%). ¹H NMR (500 MHz, DMSO-d₆) δ 8.52 (s, 1H), 8.14 (dd, J=6.3, 2.3 Hz, 1H), 8.01-7.95 (m, 1H), 7.85 (d, J=9.0 Hz, 1H), 7.81 (d, J=9.3 Hz, 1H), 7.61 (t, J=9.0 Hz, 1H), 3.26 (d, J=6.7 Hz, 2H), 2.75 (t, J=6.1 Hz, 2H), 2.03-1.92 (m, 2H), 1.79-1.68 (m, 2H); ¹³C NMR (126 MHz, DMSO) δ 163.50 (J=257.04 Hz), 153.77, 143.56, 142.81, 138.70, 137.36, 137.29, 136.44, 134.73, 129.49, 122.20, 116.85, 116.69, 116.30, 114.70, 114.42, 100.43, 100.31, 29.62, 28.60, 22.47, 22.40; HRMS (ESI) m/z calcd for C₂₁H₁₈FN₄ [M+H]⁺ 345.1515, found 345.1519.

### 2-Fluoro-5-(8,9,10,11-tetrahydro-3H-pyrrolo[3,2-a]phenanthridin-7-yl)benzonitrile

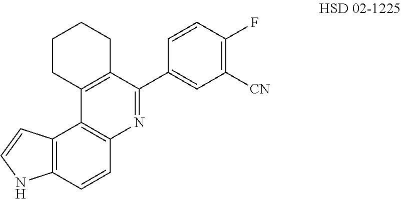 [SMILES: N#Cc1cc(-c2nc3ccc4[nH]ccc4c3c3c2CCCC3)ccc1F]

Method A: Off-white solid (131 mg, 38%). ¹H NMR (500 MHz, Methanol-d₄) δ 8.09 (dd, J=6.1, 2.3 Hz, 1H), 8.04 (d, J=9.0 Hz, 1H), 8.00 (ddd, J=8.7, 5.1, 2.3 Hz, 1H), 7.78 (d, J=9.0 Hz, 1H), 7.63-7.58 (m, 2H), 7.35-7.32 (m, 1H), 3.62 (t, 2H), 2.80 (t, J=6.2 Hz, 2H), 2.13-2.09 (m, 2H), 1.93-1.87 (m, 2H); ¹³C NMR (126 MHz, MeOD) δ 164.50, 162.42, 149.33, 138.09, 136.46, 134.57, 133.67, 128.43, 124.75, 123.69, 120.09, 119.05, 117.10, 116.70, 116.53, 112.81, 106.28, 101.63, 30.66, 27.94, 21.86, 21.54; HRMS (ESI) m/z calcd for C₂₂H₁₉FN₃ [M+H]⁺ 344.1563, found 344.1565.

### 3-Methoxy-4-(8,9,10,11-tetrahydro-3H-pyrazolo[4,3-a]phenanthridin-7-yl)phenol

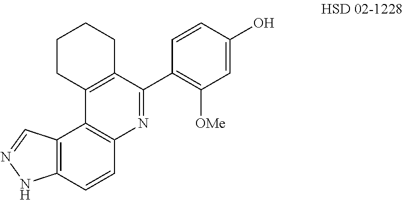 [SMILES: COc1cc(O)ccc1-c1nc2ccc3[nH]ncc3c2c2c1CCCC2]

Method A: Off-white solid (87 mg, 25%). ¹H NMR (500 MHz, DMSO-d₆) δ 9.59 (s, 1H), 8.53 (s, 1H), 7.84-7.74 (m, 2H), 6.97 (d, J=8.1 Hz, 1H), 6.50 (d, J=2.2 Hz, 1H), 6.45 (dd, J=8.1, 2.2 Hz, 1H), 3.63 (s, 3H), 3.34-3.31 (m, 1H), 3.29-3.20 (m, 1H), 2.72-2.62 (m, 1H), 2.47-2.38 (m, 1H), 2.02-1.87 (m, 2H), 1.83-1.62 (m, 2H); ¹³C NMR (126 MHz, DMSO) δ 159.02, 157.89, 155.92, 143.53, 141.09, 138.58, 136.19, 131.12, 129.60, 121.67, 121.44, 116.57, 113.84, 107.41, 99.24, 55.49, 29.45, 27.19, 22.74, 22.27; HRMS (ESI) m/z calcd for C₂₁H₂₂N₃O₂ [M+H]⁺ 348.1712, found 348.1717.

### 4-(8,9,10,11-Tetrahydro-3H-pyrrolo[3,2-a]phenanthridin-7-yl)-2-(trifluoromethyl)phenol

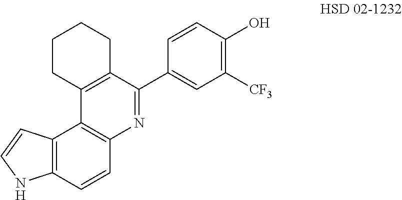 [SMILES: Oc1ccc(-c2nc3ccc4[nH]ccc4c3c3c2CCCC3)cc1C(F)(F)F]

Method A: Yellow solid (219 mg, 57%). ¹H NMR (500 MHz, DMSO-d₆) δ 11.75 (s, 1H), 10.73 (s, 1H), 7.78 (d, J=8.9 Hz, 1H), 7.67 (dq, J=3.7, 2.2 Hz, 2H), 7.63 (d, J=8.8 Hz, 1H), 7.49 (t, J=2.8 Hz, 1H), 7.14-7.08 (m, 2H), 3.37 (t, J=6.6 Hz, 3H), 2.77 (t, J=6.1 Hz, 2H), 2.03-1.93 (m, 2H), 1.78-1.68 (m, 2H); ¹³C NMR (126 MHz, DMSO) δ 155.74, 154.41, 143.42, 142.51, 134.89, 133.22, 132.30, 127.65, 127.50, 125.67 (q, J=273.42 Hz), 124.05, 123.95, 122.46, 120.32, 116.82, 116.55, 115.52, 115.28, 106.29, 29.99, 29.12, 22.83, 22.58; HRMS (ESI) n/z calcd for C₂₂H₂₀F₃N₂O [M+H]⁺ 385.1528, found 385.1530.

### 2,5-Difluoro-4-(8,9,10,11-tetrahydro-3H-pyrazolo[4,3-a]phenanthridin-7-yl)phenol

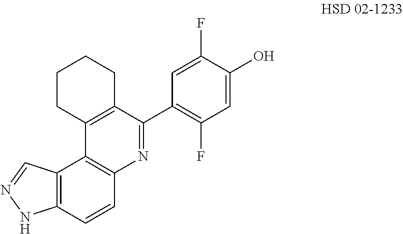 [SMILES: Oc1cc(F)c(-c2nc3ccc4[nH]ncc4c3c3c2CCCC3)cc1F]

Method A: Off-white solid (159 mg, 45%). ¹H NMR 1500 MHz, Methanol-d₄) δ 8.57 (s, 1H), 7.86 (q, J=9.1 Hz, 2H), 7.15 (dd, J=10.8, 6.7 Hz, 1H), 6.81 (dd, J=10.5, 7.1 Hz, 1H), 3.38 (s, 2H), 2.73 (s, 2H), 2.08 (p, J=6.1 Hz, 2H), 1.86 (d, J=9.8 Hz, 2H); ¹³C NMR (126 MHz, MeOD) δ 156.74 (J=239.16 Hz), 151.59, 148.92 (J=238.14 Hz), 146.61, 143.45, 143.06, 138.67, 135.89, 130.94, 128.32, 122.39, 118.20, 116.93, 116.06, 113.85, 104.69, 29.48, 27.05, 22.11, 21.80; HRMS (ESI) m/z calcd for C₂₀H₂₁₈F₂N₃O [M+H]⁺ 354.1418, found 354.1421.

### 7-(3-(Trifluoromethyl)-1H-pyrazol-4-yl)-8,9,10,11-tetrahydro-3H-pyrrolo[3,2-a]phenanthridine

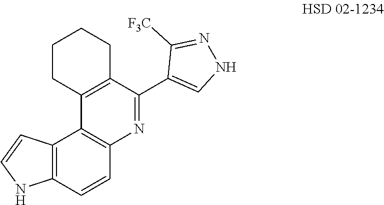 [SMILES: FC(F)(F)c1n[nH]cc1-c1nc2ccc3[nH]ccc3c2c2c1CCCC2]

Method A: Off-white solid (135 mg, 38%). ¹H NMR (500 MHz, Methanol-d₄) δ 7.92 (s, 1H), 7.79 (d, J=9.0 Hz, 1H), 7.71-7.65 (m, 1H), 7.45-7.40 (m, 1H), 7.18 (d, J=3.2 Hz, 1H), 3.46 (t, J=6.4 Hz, 2H), 2.65 (t, J=6.3 Hz, 2H), 2.01 (dp, J=9.8, 3.3 Hz, 2H), 1.85 (qd, J=6.5, 3.2 Hz, 2H); ¹³C NMR (126 MHz, MeOD) δ 146.73, 143.44, 142.52, 133.34, 130.08, 129.00, 123.12, 122.85, 121.93, 120.17, 119.38, 116.04, 105.77, 29.74, 27.88, 22.38, 21.90; HRMS (ESI) m/z calcd for C₁₉H₁₆F₃N₄ [M+H]⁺ 357.1327, found 357.1327.

### 7-(3-methyl-1H-pyrazol-4-yl)-8,9,10,11-tetrahydro-3H-pyrrolo[3,2-a]phenanthridine

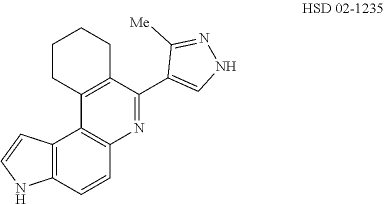 [SMILES: Cc1n[nH]cc1-c1nc2ccc3[nH]ccc3c2c2c1CCCC2]

Method A: Off-white solid (181 mg, 60%). ¹H NMR (500 MHz, DMSO-d₆) δ 8.15 (d, J=8.9 Hz, 1H), 8.09-8.02 (m, 1H), 7.74 (t, J=2.9 Hz, 1H), 7.23 (t, J=2.4 Hz, 1H), 3.46 (t, J=6.4 Hz, 2H), 2.80 (t, J=6.3 Hz, 2H), 2.30 (s, 3H), 2.03-1.94 (m, 2H), 1.84-1.74 (m, 2H); ¹³C NMR (126 MHz, DMSO) δ 144.36, 134.42, 133.65, 130.53, 126.80, 122.88, 120.88, 119.84, 113.93, 110.98, 106.60, 31.17, 27.69, 22.00, 21.57, 19.02; HRMS (ESI) m/z calcd for C₁₉H₁₉N₄ [M+H]⁺ 303.1610, found 303.1611.

### 1-Methyl-7-(3-(trifluoromethyl)-1H-pyrazol-4-yl)-8,9,10,11-tetrahydro-3H-pyrazolo[4,3-a]phenanthridine

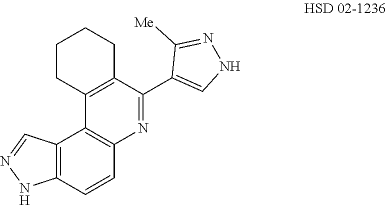 [SMILES: Cc1n[nH]cc1-c1nc2ccc3[nH]ncc3c2c2c1CCCC2]

Method A: Off-white solid (179 mg, 59%). ¹H NMR (500 MHz, DMSO-d₆) δ 8.53 (s, 1H), 7.84-7.74 (m, 3H), 3.33 (s, 3H), 2.85 (t, J=6.1 Hz, 2H), 2.33 (s, 2H), 2.00-1.97 (m, 2H), 1.81-1.77 (m, 2H); ¹³C NMR (126 MHz, DMSO) δ 151.10, 143.71, 141.83, 138.63, 136.21, 130.12, 129.59, 121.02, 118.17, 116.48, 114.04, 29.70, 28.62, 22.63; HRMS (ESI) m/z calcd for C₁₅H₁₈N₅ [M+H]⁺ 304.1562, found 304.1565.

### 1-Methyl-7-(3-(trifluoromethyl)-1H-pyrazol-4-yl)-8,9,10,11-tetrahydro-3H-pyrazolo[4,3-a]phenanthridine

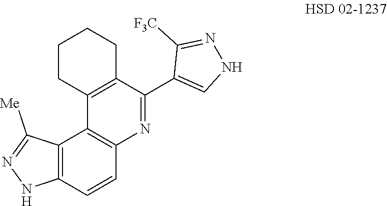 [SMILES: Cc1n[nH]c2ccc3nc(-c4c[nH]nc4C(F)(F)F)c4c(c3c12)CCCC4]

Method A: Off-white solid (150 mg, 42%). ¹H NMR (500 MHz, Methanol-d₄) δ 8.38 (d, J=0.9 Hz, 1H), 8.19 (d, J=9.2 Hz, 1H), 7.88 (d, J=9.2 Hz, 1H), 3.84-3.71 (m, 2H), 3.04 (s, 3H), 2.83-2.80 (m, 2H), 2.00-1.98 (m, 4H); ¹³C NMR (126 MHz, MeOD) δ 155.05, 144.54, 141.22, 140.08 (q=38 Hz), 135.79, 133.55, 132.43, 126.71, 124.45 (q, J=270 Hz), 123.66, 120.18, 118.65, 112.60, 109.60, 33.05, 26.90, 21.14, 20.63, 17.53; HRMS (ESI) m/z calcd for C₁₈H₁₅F₃N₅ [M+H]⁺ 358.1280, found 358.1288.

### 4-(8,9,10,11-Tetrahydro-3H-pyrrolo[3,2-a]phenanthridin-7-yl)-3-(trifluoromethyl)phenol

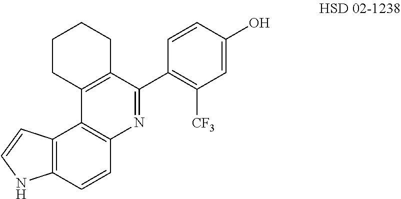 [SMILES: Oc1ccc(-c2nc3ccc4[nH]ccc4c3c3c2CCCC3)c(C(F)(F)F)c1]

Method A: Yellow solid (154 mg, 40%). ¹H NMR (500 MHz, Methanol-d₄) δ 7.78 (d, J=9.0 Hz, 1H), 7.66 (d, J=8.9 Hz, 1H), 7.40 (d, J=3.0 Hz, 1H), 7.23-7.18 (m, 2H), 7.16 (d, J=2.8 Hz, 1H), 7.11 (dd, J=8.3, 2.3 Hz, 1H), 3.43 (t, J=6.6 Hz, 2H), 2.57-2.41 (m, 2H), 2.07-1.87 (m, 2H), 1.83-1.68 (m, 2H); ¹³C NMR (126 MHz, MeOD) δ 157.58, 153.68, 143.62, 141.79, 133.27, 132.02, 129.99, 129.72, 129.38 (J=30.24 Hz), 128.34, 122.95 (J=262.08 Hz), 122.88, 121.56, 118.37, 116.02, 114.80, 112.68, 105.70, 29.74, 27.85, 22.40, 21.83; HRMS (ESI) n/z calcd for C₂₂H₂₀F₃N₂O [M+H]⁺ 385.1528, found 385.1530.

### 4-(8,9,10,11-Tetrahydro-3H-pyrazolo[4,3-a]phenanthridin-7-yl)-3-(trifluoromethyl)phenol

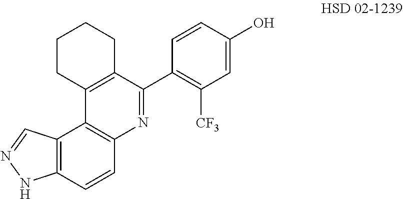 [SMILES: Oc1ccc(-c2nc3ccc4[nH]ncc4c3c3c2CCCC3)c(C(F)(F)F)c1]

Method A: Off-white solid (142 mg, 36%). ¹H NMR (500 MHz, DMSO-d₆) δ 10.24 (s, 1H), 8.55 (s, 1H), 7.84 (d, J=9.0 Hz, 1H), 7.77 (d, J=8.8 Hz, 1H), 7.24 (d, J=8.3 Hz, 1H), 7.19 (d, J=2.5 Hz, 1H), 7.11 (dd, J=8.3, 2.5 Hz, 1H), 3.44-3.24 (m, 2H), 2.57-2.50 (m, 1H), 2.34 (dt, J=17.0, 5.6 Hz, 1H), 2.00-1.83 (m, 2H), 1.82-1.62 (m, 2H); ¹³C NMR (126 MHz, DMSO) δ 157.55, 155.48, 143.04, 141.85, 138.71, 136.25, 132.68, 130.30, 129.99, 129.54, 128.57 (q, J=30.24 Hz), 125.45 (q, J=274.68 Hz), 122.04, 119.30, 116.44, 114.27, 113.02, 112.98, 29.41, 28.15, 22.58, 22.19; HRMS (ESI) m/z calcd for C₂₁H₁₉F₃N₃O [M+H]⁺ 386.1480, found 386.1488.

### 7-(5-(Trifluoromethyl)-1H-pyrazol-3-yl)-8,9,10,11-tetrahydro-3H-pyrazolo[4,3-a]phenanthridine

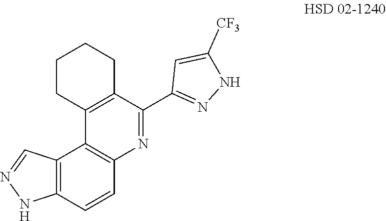 [SMILES: FC(F)(F)c1cc(-c2nc3ccc4[nH]ncc4c3c3c2CCCC3)n[nH]1]

Method A: Off-white solid (150 mg, 42%). ¹H NMR (500 MHz, DMSO-d₆) δ 8.56 (s, 1H), 7.91 (d, J=8.7 Hz, 2H), 7.17 (s, 1H), 3.33-3.28 (m, 3H), 3.00 (t, J=6.2 Hz, 2H), 2.02-1.95 (m, 2H), 1.88-1.80 (m, 2H); ¹³C NMR (126 MHz, DMSO) δ 144.59, 143.32, 142.97, 142.16, (J=36.83 Hz), 138.87, 136.55, 129.44, 125.49 (J=270.51 Hz), 122.50, 116.27, 115.12, 104.88, 29.83, 27.83, 22.26, 22.22; HRMS (ESI) m/z calcd for C₁₈H₁₅F₃N₅[M+H]⁺ 358.1280, found 358.1289.

### 4-(9-(Trifluoromethyl)-8,9,10,11-tetrahydro-3H-pyrrolo[3,2-a]phenanthridin-7-yl)phenol

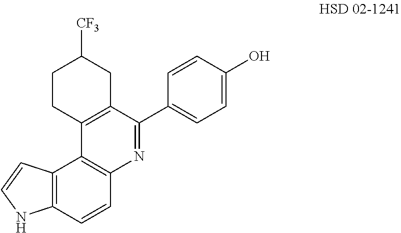 [SMILES: Oc1ccc(-c2nc3ccc4[nH]ccc4c3c3c2CC(C(F)(F)F)CC3)cc1]

Method A: Off-white solid (157 mg, 41%). ¹H NMR (500 MHz, DMSO-d₆) δ 11.93 (s, 1H), 9.74 (s, 1H), 7.84 (d, J=8.8 Hz, 1H), 7.67 (s, 1H), 7.60-7.50 (m, 1H), 7.39 (d, J=8.0 Hz, 2H), 7.21-7.07 (m, 1H), 6.99-6.83 (m, 2H), 3.65 (dd, J=18.1, 5.8 Hz, 1H), 3.01-2.93 (m, 1H), 2.89-2.81 (m, 1H), 2.78-2.65 (m, 1H), 2.43-2.31 (m, 1H), 1.92-1.77 (m, 1H); ¹³C NMR (126 MHz, DMSO) δ 157.87, 155.35, 133.27, 131.96, 130.83, 129.75, 127.53, 124.57, 124.44, 121.71, 120.23, 117.24, 115.32, 106.11, 37.63 (q=26.6 Hz), 29.04, 27.78, 21.66; HRMS (ESI) m/z calcd for C₂₂H₂₀F₃N₂O [M+H]⁺ 385.1528, found 385.1532.

### 4-(9-(Trifluoromethyl)-8,9,10,11-tetrahydro-3H-pyrazolo[4,3-a]phenanthridin-7-yl)phenol

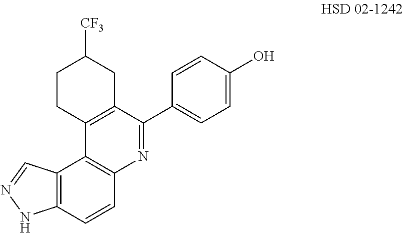 [SMILES: Oc1ccc(-c2nc3ccc4[nH]ncc4c3c3c2CC(C(F)(F)F)CC3)cc1]

Method A: Off-while solid (165 mg, 43%). ¹H NMR (500 MHz, DMSO-d₆) δ 10.27 (s, 1H), 8.60 (s, 1H), 8.11-7.90 (m, 2H), 7.57 (d, J=8.2 Hz, 2H), 7.03 (d, J=8.3 Hz, 2H), 3.54-3.43 (m, 1H), 3.40-3.28 (m, 1H), 3.10-2.97 (m, 1H), 2.90-2.82 (m, 1H), 2.79-2.68 (m, 1H), 2.39-2.30 (m, 1H), 1.94-1.79 (m, 1H); ¹³C NMR (126 MHz, DMSO) δ 159.74, 152.87, 148.17, 137.78, 131.71, 129.43 (q, J=278.46 Hz), 127.87, 122.81, 121.71, 115.86, 115.01, 37.62 (q, J=26.46 Hz), 29.44, 27.22, 20.90; HRMS (ESI) m/z calcd for C₂₁H₁₉F₃N₃O [M+H]⁺ 386.1480, found 386.1484.

### 4-(9,9-Difluoro-8,9,10,11-tetrahydro-3H-pyrrolo[3,2-a]phenanthridin-7-yl)phenol

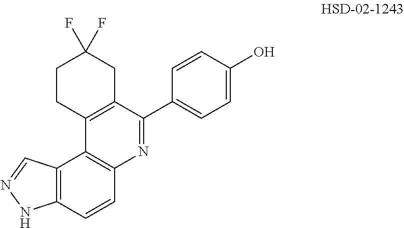 [SMILES: Oc1ccc(-c2nc3ccc4[nH]ncc4c3c3c2CC(F)(F)CC3)cc1]

Method A: Off-white solid (152 mg, 43%). ¹H NMR (500 MHz, DMSO-d₆) δ 9.71 (s, 1H), 8.59 (s, 1H), 7.90 (d, J=9.1 Hz, 1H), 7.87-7.81 (m, 1H), 7.41 (d, J=8.5 Hz, 2H), 6.89 (d, J=8.5 Hz, 2H), 3.63-3.55 (m, 2H), 3.41-3.36 (m, 2H), 2.55-2.50 (m, 2H); ¹³C NMR (126 MHz, DMSO) δ 157.94, 156.62, 144.16, 139.61, 138.71, 136.27, 131.03, 130.86, 129.49, 125.65, 124.54, 123.75, 121.86, 120.55, 116.30, 115.37, 115.11, 37.18 (J=26.46 Hz), 29.37 (J=23.94 Hz), 28.01; HRMS (ESI) m/z calcd for C₂₀H₁₈F₂N₃O [M+H]⁺ 354.1418, found 354.1421.

### 4-(9,9-Difluoro-8,9,10,11-tetrahydro-3H-pyrrolo[3,2-a]phenanthridin-7-yl)phenol

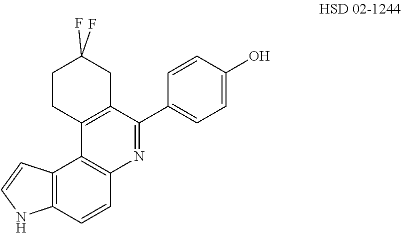 [SMILES: Oc1ccc(-c2nc3ccc4[nH]ccc4c3c3c2CC(F)(F)CC3)cc1]

Method A: Off-white solid (180 mg, 49%). ¹H NMR (500 MHz, DMSO-d₆) δ 11.84 (s, 1H), 7.84 (d, J=8.8 Hz, 1H), 7.67 (d, J=8.8 Hz, 1H), 7.53 (d, J=3.0 Hz, 1H), 7.39 (d, J=8.1 Hz, 2H), 7.13 (d, J=3.1 Hz, 1H), 6.88 (d, J=8.1 Hz, 2H), 3.63 (t, J=7.1 Hz, 2H), 3.33 (t, J=14.5 Hz, 2H), 2.48-2.38 (m, 2H); ¹³C NMR (126 MHz, DMSO) δ 157.78, 155.53, 143.87, 139.53, 133.29, 131.52, 130.80, 125.76, 124.38, 123.95, 123.87, 122.78, 121.98, 121.20, 120.23, 117.15, 115.32, 106.13, 37.50 (J=26.46 Hz), 29.55 (J=23.94 Hz), 28.22; HRMS (ESI) m/z calcd for C₂₁H₁₉F₂N₂O [M+H]⁺ 353.1465, found 353.1470.

### Ethyl 8,9,10,11-tetrahydro-3H-pyrazolo[4,3-a]phenanthridine-7-carboxylate

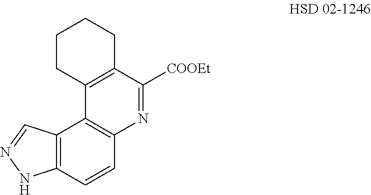 [SMILES: CCOC(=O)c1nc2ccc3[nH]ncc3c2c2c1CCCC2]

Method A: Off-white solid (151 mg, 51%). ¹H NMR (500 MHz, DMSO-d₆) δ 8.58 (s, 1H), 7.90 (d, J=9.1 Hz, 1H), 7.85 (d, J=9.1 Hz, 1H), 4.38 (q, J=7.1 Hz, 2H), 2.94 (t, J=6.3 Hz, 2H), 1.98 (tt, J=7.7, 6.0, 4.6 Hz, 2H), 1.84 (dd, J=7.6, 3.9 Hz, 2H), 1.34 (t, J=7.1 Hz, 3H). ¹³C NMR (126 MHz, DMSO) δ 167.34, 148.23, 143.50, 142.89, 139.08, 136.66, 129.37, 128.75, 123.47, 116.23, 115.28, 61.64, 29.54, 26.54, 22.29, 21.87, 14.60; HRMS (ESI) m/z calcd for C₁₇H₂₀N₃O₂ [M+H]⁺ 298.1556, found 298.1562.

### 7-(4-Hydroxyphenyl)-8,9,10,11-tetrahydro-3H-pyrrolo[3,2-a]phenanthridine-1-carbonitrile

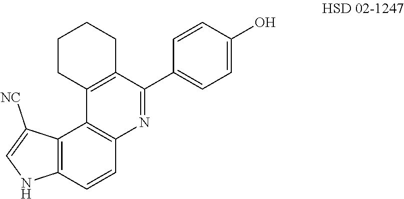 [SMILES: N#Cc1c[nH]c2ccc3nc(-c4ccc(O)cc4)c4c(c3c12)CCCC4]

Method A: Yellow solid (140 mg, 41%). Method A: Off-white solid (182 mg, 56%). ¹H NMR (500 MHz, DMSO-d₆) δ 10.36 (s, 1H), 8.70 (d, J=3.1 Hz, 1H), 8.27 (d J=8.9 Hz, 1H), 8.19 (d, J=9.1 Hz, 1H), 7.59 (d, J=8.0 Hz, 2H), 7.04 (d, J=8.1 Hz, 2H), 3.77 (t, J=6.3 Hz, 2H), 2.84 (t, J=6.3 Hz, 2H), 1.99-1.91 (m, 2H), 1.87-1.77 (m, 2H); ¹³C NMR (126 MHz, DMSO) δ 160.39, 153.97, 152.19, 138.86, 135.26, 135.01, 131.92, 130.79, 123.33, 122.29, 121.56, 119.23, 117.69, 117.36, 115.99, 89.30, 33.48, 27.99, 21.34, 21.30; HRMS (ESI) m/z calcd for C₂H₂₀N₃O [M+H]⁺ 342.1606, found 342.1611.

### 4-(7,8,9,10-Tetrahydrophenanthridin-6-yl)phenol

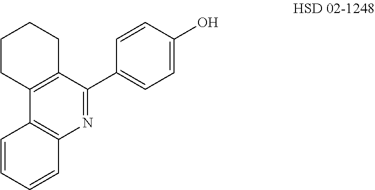 [SMILES: Oc1ccc(-c2nc3ccccc3c3c2CCCC3)cc1]

Method A: Off-white solid (75 mg, 20%). ¹H NMR (500 MHz, Methanol-d₄) δ 8.42 (d, J=8.5 Hz, 1H), 8.16 (d, J=8.5 Hz, 1H), 8.04 (t, J=7.7 Hz, 1H), 7.92 (t, J=7.8 Hz, 1H), 7.60 (d, J=8.2 Hz, 2H), 7.07 (d, J=8.3 Hz, 2H), 3.52 (t, J=6.5 Hz, 2H), 2.90 (t, J=6.2 Hz, 2H), 2.12-2.06 (m, 2H), 1.95-1.84 (m, 2H); ¹³C NMR (126 MHz, MeOD) δ 160.77, 156.16, 155.60, 135.74, 132.96, 131.15, 130.92, 129.04, 127.05, 124.04, 122.18, 120.19, 115.59, 27.66, 26.87, 21.56, 20.93; HRMS (ESI) m/z calcd for C₁₉H₂₀NO [M+H]⁺ 378.1545, found 378.1546.

### 4-(6,7,8,9-Tetrahydro-3H-pyrrolo[3,2-c]phenanthridin-5-yl)phenol

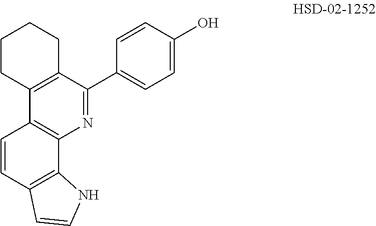 [SMILES: Oc1ccc(-c2nc3c(ccc4cc[nH]c43)c3c2CCCC3)cc1]

Method A: Pale yellow solid (119 mg, 38%). ¹H NMR (500 MHz, Methanol-d₄) δ 7.73 (d, J=8.8 Hz, 1H), 7.58 (d, J=8.8 Hz, 1H), 7.39-7.32 (m, 3H), 6.96-6.89 (m, 2H), 6.62 (d, J=3.0 Hz, 1H), 3.24 (tt, J=6.6, 1.2 Hz, 2H), 2.72 (t, J=6.3 Hz, 2H), 1.95 (td, J=6.5, 3.0 Hz, 2H), 1.82-1.73 (m, 2H); ¹³C NMR (126 MHz, MeOD) δ 157.94, 157.43, 143.79, 134.49, 131.77, 130.32, 129.91, 126.79, 126.36, 123.54, 122.78, 120.97, 114.65, 113.55, 102.78, 28.24, 26.19, 22.54, 22.16; HRMS (ESI) m/z calcd for C₂₁H₁₉N₂O [M+H]⁺ 315.1497, found 315.1498.

### 4-(3-(2-Hydroxyethyl)-8,9,10,11-tetrahydro-3H-pyrazolo[4,3-a]phenanthridin-7-yl)phenol

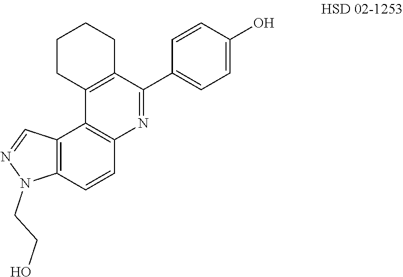 [SMILES: OCCn1ncc2c3c4c(c(-c5ccc(O)cc5)nc3ccc21)CCCC4]

Method A: Off-white solid (177 mg, 49%). ¹H NMR (500 MHz, DMSO-d) δ 9.59 (s, 1H), 8.51 (s, 1H), 7.96 (d, J=9.1 Hz, 1H), 7.81 (d, J=9.1 Hz, 1H), 7.39 (d, J=8.5 Hz, 2H), 6.86-6.82 (m, 2H), 4.55 (t, J=5.6 Hz, 2H), 3.88-3.82 (m, 2H), 3.32-3.27 (m, 2H), 2.79 (t, J=6.2 Hz, 2H), 2.01-1.96 (m, 2H), 1.77-1.70 (m, 2H); ¹³C NMR (126 MHz, DMSO) δ 157.58, 157.01, 143.57, 142.09, 138.61, 135.29, 131.97, 130.89, 129.53, 129.38, 121.31, 117.15, 115.09, 114.17, 60.84, 51.76, 29.69, 29.23, 22.64; HRMS (ESI) m/z calcd for C₁₂H₂₄N₃O₂ [M+H]⁺ 362.1869, found 362.1871.

7-(3-(δ 8.26 (s, 1H), 7.95 (d, J=9.1 Hz, 1H), 7.86 (d, J=9.1 Hz, 1H), 3.55 (t, J=6.3 Hz, 2H), 2.72 (t, J=6.3 Hz, 2H), 1.95-1.86 (m, 2H), 1.84-1.77 (m, 2H); ¹³C NMR (126 MHz, DMSO) δ 150.24, 144.55, 143.03, 140.24, 139.34, 131.66, 131.35, 130.73, 125.49 (J=2704.68 Hz). 121.28, 121.28, 119.78, 119.14, 117.84, 116.46, 114.90, 31.80, 28.11, 21.99; HRMS (ESI) m/z calcd for C₁₉H₁₄F₃N₆ [M+H]⁺ 383.1232, found 383.1238.

### 6-(8,9,10,11-Tetrahydro-3H-pyrrolo[3,2-a]phenanthridin-7-yl)benzo[d]thiazol-2-amine

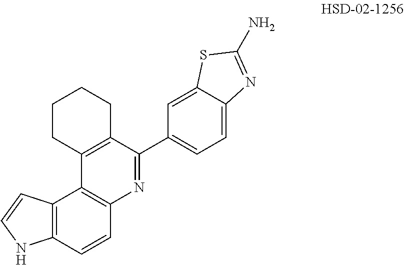 [SMILES: Nc1nc2ccc(-c3nc4ccc5[nH]ccc5c4c4c3CCCC4)cc2s1]

Method A: Pale yellow solid (115 mg, 31%). ¹H NMR (500 MHz, DMSO-d₆) δ 7.83 (t, J=1.2 Hz, 1H), 7.77 (dd, J=8.8, 0.8 Hz, 1H), 7.62 (d, J=8.7 Hz, 1H), 7.52 (s, 2H), 7.49 (t, J=2.8 Hz, 1H), 7.42-7.36 (m, 2H), 7.15-7.09 (m, 1H), 3.38 (t, J=6.5 Hz, 2H), 2.80 (t, J=6.1 Hz, 2H), 2.01-1.93 (m, 2H), 1.78-1.68 (m, 2H); ¹³C NMR (126 MHz, DMSO) δ 167.48, 155.93, 152.75, 143.42, 142.23, 134.61, 133.18, 131.13, 127.69, 127.23, 124.12, 123.91, 122.33, 122.08, 120.39, 117.29, 116.41, 106.26, 30.02, 29.30, 22.94, 22.64; HRMS (ESI) nm/z calcd for C₂₂H₁₉N₄S [M+H]⁺ 371.1330, found 371.1339.

### 6-(3,8,9,10-Tetrahydrocyclopenta[c]pyrazolo[4,3-f]quinolin-7-yl)benzo[d]thiazol-2-amine

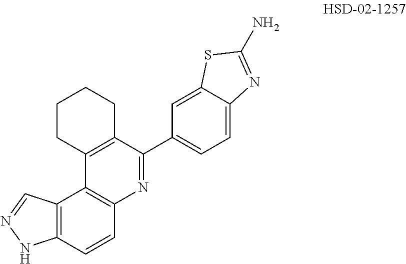 [SMILES: Nc1nc2ccc(-c3nc4ccc5[nH]ncc5c4c4c3CCCC4)cc2s1]

Method A: Yellow solid (118 mg, 33%). ¹H NMR (500 MHz, DMSO-d₆) δ 8.44 (s, 1H), 8.20 (d, J=1.8 Hz, 1H), 7.90 (d, J=9.2 Hz, 1H), 7.84 (d, J=9.0 Hz, 1H), 7.77 (dd, J=8.3, 1.9 Hz, 1H), 7.60 (s, 2H), 7.43 (d, J=8.3 Hz, 1H), 3.48 (t, J=7.5 Hz, 2H), 3.29 (t, J=7.6 Hz, 2H), 2.25 (p. J=7.5 Hz, 2H); ¹³C NMR (126 MHz, DMSO) δ 167.94, 153.47, 152.28, 149.49, 144.86, 138.15, 135.97, 135.08, 133.41, 131.62, 129.24, 126.75, 121.54, 118.85, 117.60, 116.83, 114.58, 33.57, 33.43, 25.01; HRMS (ESI) m/z calcd for C₂₀H₁₆N₅S [M+H]⁺ 358.1126, found 358.1128.

### N-(4-(2-methyl-8,9,10,11-tetrahydro-3H-pyrrolo[3,2-a]phenanthridin-7-yl)phenyl)methanesulfonamide

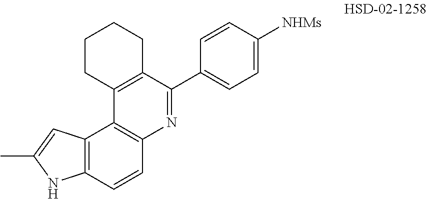

Method A: Pale yellow solid (205 mg, 50%). ¹H NMR (500 MHz, DMSO-d₆) δ 7.24 (d, J=8.9 Hz, 1H), 6.92 (d, J=8.9 Hz, 1H), 6.86 (d, J=8.3 Hz, 2H), 6.70 (d, J=8.3 Hz, 2H), 6.28 (s, 1H), 2.86-2.78 (m, 2H), 2.29 (s, 3H), 2.11-2.02 (m, 2H), 1.81 (s, 2H), 1.39-1.28 (m, 2H), 1.14-1.04 (m, 2H); ¹³C NMR (126 MHz, DMSO) δ 154.13, 148.50, 140.10, 136.74, 132.90, 129.61, 128.08, 126.79, 122.08, 120.26, 119.11, 118.09, 110.65, 104.05, 37.63, 30.40, 26.94, 20.76, 20.43, 11.29; HRMS (ESI) m/z calcd for C₂₃H₂₈N₃O₂S [M+H]⁺ 410.1902, found 410.1907.

### N-(4-(3,8,9,10-Tetrahydrocyclopenta[c]pyrrolo[3,2-f]quinolin-7-yl)phenyl)methanesulfonamide

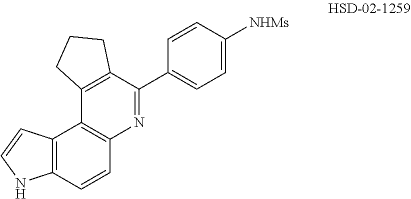

Method A: Off-white solid (179 mg, 47%). ¹H NMR (500 MHz, DMSO-d₆) δ 11.69 (t, J=2.3 Hz, 1H), 9.91 (s, 1H), 7.89-7.84 (m, 2H), 7.77 (dd, J=8.9, 0.8 Hz, 1H), 7.70 (d, J=8.9 Hz, 1H), 7.48 (t, J=2.7 Hz, 1H), 7.37-7.29 (m, 2H), 7.01-6.96 (m, 11H), 3.50 (t, J=7.5 Hz, 2H), 3.24 t, =7.5 z, 2H), 3.05 (s, 3H), 2.24 (p, J=7.6 Hz, 2H); ¹³C NMR (126 MHz, DMSO) δ 150.60, 149.35, 144.61, 138.77, 136.41, 134.23, 132.57, 129.96, 124.46, 123.67, 120.85, 120.10, 119.48, 116.78, 104.29, 33.87, 33.12, 25.08; HRMS (ESI) m/z calcd for C₂₁H₂₂N₃O₂S [M+H]⁺380.1433, found 380.1440.

### 7-(2-Aminobenzo[d]thiazol-6-yl)-8,9,10,11-tetrahydro-3H-pyrrolo[3,2-a]phenanthridine-1-carbonitrile

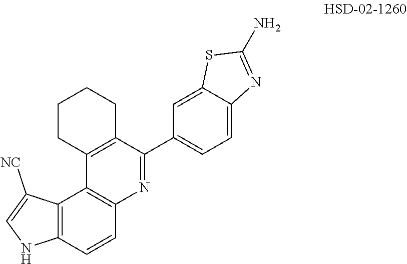 [SMILES: N#Cc1c[nH]c2ccc3nc(-c4ccc5nc(N)sc5c4)c4c(c3c12)CCCC4]

Method A: Pale yellow solid (119 mg, 30%). ¹H NMR (500 MHz, DMSO-d₆) δ 9.19 (s, 2H), 8.69 (d, J=3.1 Hz, 1H), 8.29-8.20 (m, 3H), 7.71 (dd, J=8.3, 1.8 Hz, 1H), 7.66 (d, J=8.3 Hz, 1H), 3.75 (t, J=6.3 Hz, 2H), 2.83 (t, J=6.4 Hz, 2H), 2.02-1.91 (m, 2H), 1.86-1.72 (m, 2H); ¹³C NMR (126 MHz, DMSO) δ 169.71, 154.01, 151.40, 138.84, 135.50, 135.06, 130.71, 128.82, 128.33, 126.00, 124.16, 123.58, 121.67, 119.22, 117.85, 117.28, 116.11, 114.43, 89.32, 33.49, 27.97, 21.30; HRMS (ESI) m/z calcd for C₂₃H₁₈N₅S [M+H]⁺ 396.1283, found 396.1288.

### 6-(1-Methyl-8,9,10,11-tetrahydro-3H-pyrazolo[4,3-a]phenanthridin-7-y)benzo[d]thiazol-2-amine

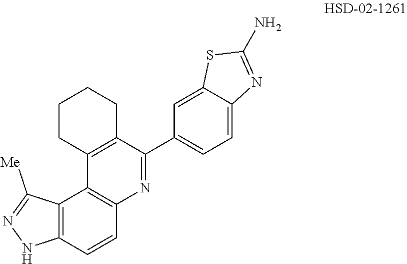 [SMILES: Cc1n[nH]c2ccc3nc(-c4ccc5nc(N)sc5c4)c4c(c3c12)CCCC4]

Method A: Off-white solid (116 mg, 30%). ¹H NMR (500 MHz, Methanol-d₄) δ 7.77 (d, J=9.1 Hz, 1H), 7.75-7.68 (m, 2H), 7.53-7.47 (m, 1H), 7.38 (dd, J=8.2, 1.8 Hz, 1H), 3.52-3.42 (m, 2H), 2.90 (s, 3H), 2.81 (q, J=4.3, 3.1 Hz, 2H), 1.89-1.82 (m, 4H); ¹³C NMR (126 MHz, MeOD) δ 169.08, 156.70, 151.68, 143.60, 143.42, 143.25, 141.25, 133.99, 130.75, 128.98, 128.59, 126.61, 123.95, 121.19, 116.95, 115.76, 113.89, 31.88, 27.83, 22.07, 21.90, 17.96; HRMS (ESI) m/z calcd for C₂₂H₂₀N₅S [M+H]⁺ 386.1439, found 386.1447.

### 7-(3,5-Dimethyl-1H-pyrazol-4-yl)-8,9,10,11-tetrahydro-3H-pyrazolo[4,3-a]phenanthridine

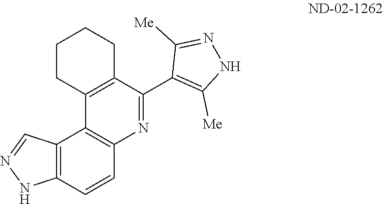 [SMILES: Cc1n[nH]c(C)c1-c1nc2ccc3[nH]ncc3c2c2c1CCCC2]

Method A: Off-white solid (193 mg, 60%). ¹H NMR (500 MHz, DMSO-d₆) δ 8.58-8.53 (m, 1H), 7.82 (dd, J=9.1, 0.9 Hz, 1H), 7.76 (d, J=9.0 Hz, 1H), 3.30 (t, J=6.5 Hz, 2H), 2.56 (t, J=6.2 Hz, 2H), 2.00 (s, 9H), 1.76 (qd, J=8.6, 7.2, 4.1 Hz, 2H); ¹³C NMR (126 MHz, DMSO) δ 151.69, 144.07, 141.77, 139.31, 135.31, 131.30, 129.63, 121.60, 117.69, 116.32, 114.93, 29.51, 27.72, 22.74, 22.45; HRMS (ESI) m/z calcd for C₁₉H₂₄N₅ [M+H]⁺ 322.2032, found 322.2036.

### 7-(1H-Indazol-5-yl)-1-methyl-8,9,10,11-tetrahydro-3H-pyrazolo[4,3-a]phenanthridine

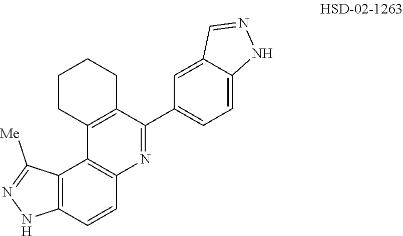 [SMILES: Cc1n[nH]c2ccc3nc(-c4ccc5[nH]ncc5c4)c4c(c3c12)CCCC4]

Method A: Off-white solid (161 mg, 45%). ¹H NMR (500 MHz, DMSO-d₆) δ 8.20 (s, 1H), 8.05 (s, 1H), 7.97 (d, J=8.9 Hz, 1H), 7.91 (d, J=9.2 Hz, 1H), 7.71 (d, J=8.6 Hz, 1H), 7.59 (dd, J=8.6, 1.6 Hz, 1H), 3.51 (q, J=7.4, 6.5 Hz, 2H), 2.93-2.87 (m, 2H), 2.79 (t, J=6.2 Hz, 2H), 1.88-1.73 (m, 4H); ¹³C NMR (126 MHz, DMSO) ¹³C NMR (126 MHz, DMSO) δ 153.72, 148.28, 140.25, 139.38, 136.90, 134.68, 130.29, 127.67, 124.60, 123.00, 122.54, 114.31, 110.60, 32.28, 27.98, 22.08, 21.72; HRMS (ESI) m/z calcd for C₂₂H₂₄N₅ [M+H]⁺ 358.2032, found 358.2033.

### 7-(1H-Indazol-5-yl)-1,2-dimethyl-8,9,10,11-tetrahydro-2H-pyrazolo[4,3-a]phenanthridine

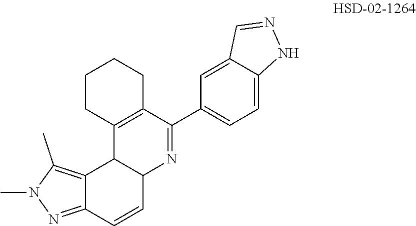 [SMILES: Cc1c2c(nn1C)C=CC1N=C(c3ccc4[nH]ncc4c3)C3=C(CCCC3)C21]

Method A: Off-white solid (159 mg, 43%). ¹H NMR (500 MHz, DMSO-d₆) δ 8.12 (s, 1H), 7.93 (dd, J=1.6, 0.8 Hz, 1H), 7.75 (d, J=9.0 Hz, 1H), 7.60 (dt, J=8.6, 1.0 Hz, 1H), 7.55 (dd, J=8.6, 1.6 Hz, 1H), 7.38 (d, J=9.0 Hz, 1H), 4.10 (s, 3H), 3.61 (t, J=6.6 Hz, 3H), 2.79 (t, J=6.2 Hz, 2H), 2.63 (s, 3H), 1.94-1.89 (m, 2H), 1.75-1.65 (m, 3H); ¹³C NMR (126 MHz, DMSO) δ 158.06, 147.33, 144.38, 143.84, 139.78, 134.44, 133.66, 132.84, 128.49, 128.17, 123.76, 123.05, 121.87, 121.40, 118.85, 118.33, 109.89, 37.78, 30.37, 29.10, 22.71, 22.60, 9.69; HRMS (ESI) m/z calcd for C₂₁H₂₄N₅[M+H]⁺ 370.2032, found 370.2039.

### 7-(1H-indazol-5-yl)-2-methyl-8,9,10,11-tetrahydro-3H-pyrrolo[3,2-a]phenanthridine

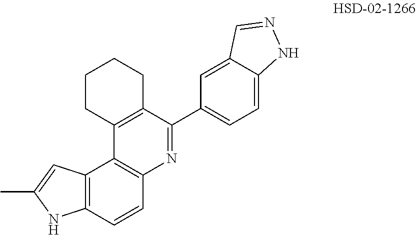 [SMILES: Cc1cc2c(ccc3nc(-c4ccc5[nH]ncc5c4)c4c(c32)CCCC4)[nH]1]

Method A: Off-white solid (146 mg, 41%). ¹H NMR (500 MHz, Methanol-d₄) δ 8.13 (s, 1H), 7.89 (t, J=1.1 Hz, 1H), 7.69 (d, J=8.9 Hz, 1H), 7.67-7.62 (m, 2H), 7.52 (dd, J=8.5, 1.5 Hz, 1H), 6.89 (s, 1H), 3.46 (t, J=6.5 Hz, 2H), 2.74 (t, J=6.2 Hz, 2H), 2.54 (s, 3H), 2.07-1.98 (m, 3H), 1.85-1.75 (m, 2H); ¹³C NMR (126 MHz, MeOD) δ 156.00, 143.82, 142.24, 139.73, 133.98, 133.79, 133.15, 127.95, 127.34, 122.81, 122.28, 121.16, 120.90, 120.51, 115.34, 109.38, 104.02, 29.95, 28.71, 22.50, 22.19, 12.08; HRMS (ESI) m/z calcd for C₂₃H₂₅N₄ [M+H]⁺ 357.2079, found 357.2079.

### 7-(1H-Indazol-5-yl)-1-methyl-8,9,10,11-tetrahydro-3H-pyrazolo[4,3-a]phenanthridine

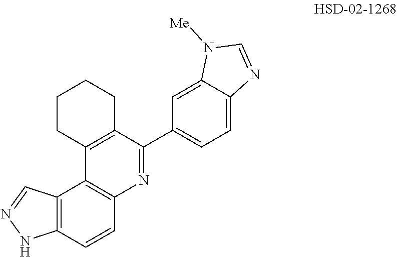 [SMILES: Cn1cnc2ccc(-c3nc4ccc5[nH]ncc5c4c4c3CCCC4)cc21]

Method A: Off-white solid (163 mg, 46%). ¹H NMR (500 MHz, MeOD-d₄) δ 9.51 (s, 1H), 8.8 (s, 1H), 8.33-8.31 (m, 2H), 8.24 (d, J=8.5 Hz, 1H), 8.13 (d, J=9.5 Hz, 1H), 8.00 (d, J=7.7 Hz, 1H), 4.25 (s, 3H), 3.69 (t, J=6.2 Hz, 1H), 2.91 (t, J=5.9 Hz, 2H), 2.24-2.19 (m, 2H), 1.97-1.92 (m, 2H); ¹³C NMR (126 MHz, MeOD) δ 156.46, 155.04, 149.36, 143.88, 134.90, 134.50, 133.73, 132.46, 132.17, 129.92, 127.08, 124.13, 121.16, 119.24, 116.83, 114.80, 113.44, 32.47, 30.98, 27.74, 21.22, 21.10; HRMS (ESI) m/z calcd for C₂₂H₂₀N₅ [M+H]⁺ 354.1719, found 354.1722.

### 7-(1H-Pyrrolo[3,2-c]pyridin-3-yl)-8,9,10,11-tetrahydro-3H-pyrazolo[4,3-a]phenanthridine

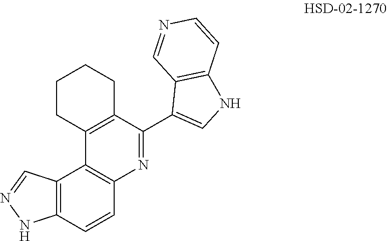 [SMILES: c1cc2[nH]cc(-c3nc4ccc5[nH]ncc5c4c4c3CCCC4)c2cn1]

Method A: Off-white solid (129 mg, 38%). ¹H NMR (500 MHz, DMSO-d₆) δ 11.82 (s, 1H), 9.49 (d, J=1.8 Hz, 1H), 8.57 (s, 1H), 8.23 (dd, J=5.7, 2.2 Hz, 1H), 7.93 (d, J=2.1 Hz, 1H), 7.89-7.82 (m, 2H), 7.44 (dd, J=5.9, 1.8 Hz, 1H), 3.33 (s, 17H), 3.07 (t, J=6.2 Hz, 2H), 2.04 (dtt, J=12.3, 6.4, 2.2 Hz, 2H), 1.89-1.78 (m, 2H); ¹³C NMR (126 MHz, DMSO) δ 151.34, 145.38, 143.85, 142.15, 140.91, 139.91, 129.62, 129.54, 128.05, 124.66, 120.76, 116.46, 115.21, 107.29, 29.95, 28.95, 22.80, 22.62; HRMS (ESI) m/z calcd for C₂₁H₁₈N₅ [M+H]⁺ 340.1562, found 340.1566.

### 7-(4-Chloro-1H-pyrrolo[2,3-b]pyridin-3-yl)-8,9,10,11-tetrahydro-3H-pyrazolo[4,3-a]phenanthridine

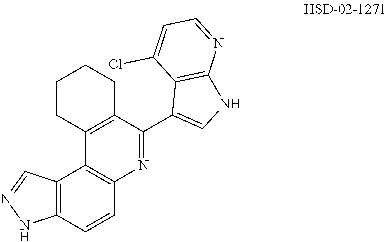 [SMILES: Clc1ccnc2[nH]cc(-c3nc4ccc5[nH]ncc5c4c4c3CCCC4)c12]

Method A: Off-white solid (180 mg, 48%). ¹H NMR (500 MHz, Methanol-d₄) δ 8.61 (s, 1H), 8.19 (d, J=5.5 Hz, 1H), 7.87 (q, J=9.3 Hz, 2H), 7.61 (s, 1H), 7.14 (d, J=5.1 Hz, 1H), 3.40 (s, 3H), 2.75-2.66 (m, 2H), 2.06 (p, J=6.3, 5.7 Hz, 2H), 1.89-1.80 (m, 2H); ¹³C NMR (126 MHz, MeOD) δ 151.51, 148.69, 143.14, 142.97, 142.52, 136.07, 135.88, 132.62, 128.15, 126.03, 122.42, 117.52, 116.46, 116.07, 113.67, 29.52, 28.07, 22.18, 21.86; HRMS (ESI) m/z calcd for C₂₁H₁₇ClN₅ [M+H]⁺ 374.1172, found 374.1176.

### 7-(1H-Pyrrolo[2,3-b]pyridin-3-yl)-8,9,10,11-tetrahydro-3H-pyrrolo[3,2-a]phenanthridine

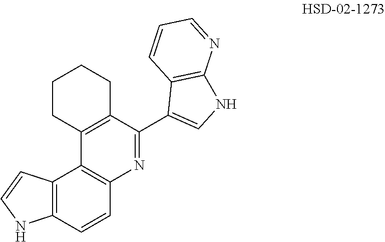 [SMILES: c1cnc2[nH]cc(-c3nc4ccc5[nH]ccc5c4c4c3CCCC4)c2c1]

Method A: Off-white solid (142 mg, 42%). ¹H NMR (500 MHz, DMSO-d₆) δ 11.92 (s, 1H), 11.72 (s, 1H), 8.57 (dd, J=7.9, 1.7 Hz, 1H), 8.27 (dd, J=4.7, 1.7 Hz, 1H), 7.90 (s, 1H), 7.77 (d, J=8.8 Hz, 1H), 7.68 (d, J=8.9 Hz, 1H), 7.47 (t, J=2.4 Hz, 1H), 7.18-7.12 (m, 1H), 7.11 (d, J=3.0 Hz, 1H), 3.40 (t, J=6.5 Hz, 2H), 3.05 (t, J=6.1 Hz, 2H), 2.05-1.97 (m, 2H), 1.81 (ddt, J=9.3, 6.3, 2.8 Hz, 2H); ¹³C NMR (126 MHz, DMSO) δ 150.58, 148.89, 143.52, 143.39, 142.01, 133.07, 130.54, 127.92, 127.10, 124.02, 123.76, 121.43, 120.44, 120.16, 116.48, 116.27, 114.39, 106.19, 30.22, 28.95, 22.89, 22.82; HRMS (ESI) m/z calcd for C₂₂H₁₉N₄ [M+H]⁺ 339.1610, found 339.1615.

### 7-(5H-Pyrrolo[3,2-d]pyrimidin-7-yl)-8,9,10,11-tetrahydro-3H-pyrazolo[4,3-a]phenanthridine

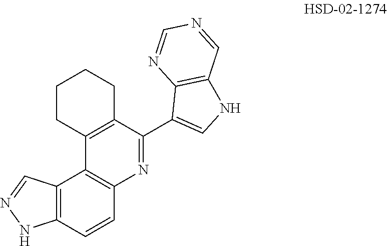 [SMILES: c1ncc2[nH]cc(-c3nc4ccc5[nH]ncc5c4c4c3CCCC4)c2n1]

Method A: Off-white solid (171 mg, 50%). ¹H NMR (500 MHz, DMSO-d₆) δ 9.72 (s, 1H), 8.83 (s, 1H), 8.55 (s, 1H), 8.07 (s, 1H), 7.93 (d, J=9.2 Hz, 1H), 7.89-7.83 (m, 1H), 3.39-3.33 (m, 2H), 3.11 (t, J=6.1 Hz, 2H), 2.08-2.01 (m, 2H), 1.91-1.81 (m, 2H); ¹³C NMR (126 MHz, DMSO) δ 151.93, 151.87, 150.53, 143.67, 142.83, 142.42, 138.64, 136.29, 129.63, 129.46, 128.35, 120.83, 118.56, 116.50, 114.42, 29.99, 28.80, 22.74, 22.53; HRMS (ESI) m/z calcd for C₂₀H₁₇N₆ [M+H]⁺ 341.1515, found 341.1517.

### 7-(6-Chloro-1H-pyrrolo[2,3-b]pyridin-3-yl)-8,9,10,11-tetrahydro-3H-pyrazolo[4,3-a]phenanthridine

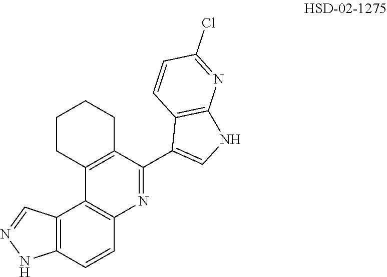 [SMILES: Clc1ccc2c(-c3nc4ccc5[nH]ncc5c4c4c3CCCC4)c[nH]c2n1]

Method A: Off-white solid (183 mg, 49%). ¹H NMR (500 MHz, DMSO-d₆) δ 11.82 (s, 1H), 9.50 (d, J=1.1 Hz, 1H), 8.55 (s, 1H), 8.23 (d, J=5.6 Hz, 1H), 7.96-7.78 (m, 3H), 7.44 (dd, J=5.6, 1.1 Hz, 1H), 3.35 (s, 2H), 3.07 (t, J=6.1 Hz, 2H), 2.04 (tdd, J=9.2, 5.5, 2.2 Hz, 2H), 1.84 (qd, J=8.8, 7.3, 4.1 Hz, 2H); ¹³C NMR (126 MHz, DMSO) δ 151.36, 145.39, 143.74, 142.15, 140.92, 139.90, 138.58, 136.23, 129.62, 128.04, 127.85, 124.65, 120.72, 116.59, 115.20, 114.23, 107.27, 29.96, 28.95, 22.80, 22.62; HRMS (ESI) m/z calcd for C₂₁H₁₇C₁N₅ [M+H]+ 374.1172, found 374.1178.

### 7-(1H-Pyrrolo[3,2-b]pyridin-3-yl)-8,9,10,11-tetrahydro-3H-pyrazolo[4,3-a]phenanthridine

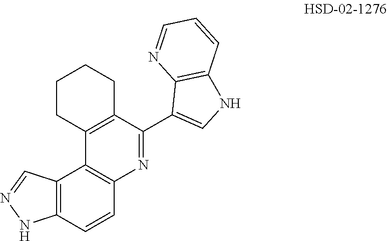 [SMILES: c1cnc2c(-c3nc4ccc5[nH]ncc5c4c4c3CCCC4)c[nH]c2c1]

Method A: Off-white solid (153 mg, 45%). ¹H NMR (500 MHz, DMSO-d₆) δ 11.60 (d, J=3.1 Hz, 1H), 8.56 (s, 1H), 8.32 (dq, J=4.3, 1.8 Hz, 1H), 7.88-7.77 (m, 4H), 7.17-7.10 (m, 1H), 3.38-3.33 (m, 2H), 2.94 (t, J=6.1 Hz, 2H), 2.03-1.93 (m, 2H), 1.77-1.65 (m, 2H); ¹³C NMR (126 MHz, DMSO) δ 152.37, 144.53, 143.65, 142.86, 141.35, 138.56, 136.22, 131.63, 129.70, 129.50, 128.91, 121.53, 119.42, 117.39, 116.76, 116.64, 113.84, 29.65, 27.96, 22.77, 22.43; HRMS (ESI) m/z calcd for C₂₁H₁₈N₅[M+H]⁺ 340.1562, found 340.1567.

### 4-(8,9,10,11-Tetrahydro-3H-pyrazolo[4,3-a]phenanthridin-7-yl)benzenesulfonamide

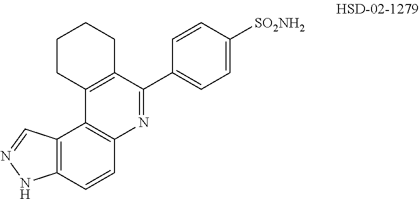 [SMILES: NS(=O)(=O)c1ccc(-c2nc3ccc4[nH]ncc4c3c3c2CCCC3)cc1]

Method A: Off-white solid (197 mg, 52%). ¹H NMR (500 MHz, DMSO-d₆) δ 8.59 (s, 1H), 7.93 (d, J=8.4 Hz, 2H), 7.87 (dd, J=9.1, 0.9 Hz, 1H), 7.80 (d, J=9.1 Hz, 1H), 7.75 (d, J=8.5 Hz, 2H), 3.32 (t, J=6.5 Hz, 2H), 2.76 (t, J=6.1 Hz, 2H), 2.03-1.95 (m, 2H), 1.78-1.69 (m, 2H); ¹³C NMR (126 MHz, DMSO) δ 155.52, 144.57, 143.84, 143.78, 142.71, 139.77, 135.26, 130.10, 129.57, 129.20, 125.83, 122.19, 116.19, 115.57, 31.16, 29.66, 28.81, 22.54, 22.43; HRMS (ESI) m/z calcd for C₂₁H₁₉N₄O₂S [M+H]⁺ 379.1229, found 379.1234.

### 4-(8,9,10,11-Tetrahydro-3H-pyrrolo[3,2-a]phenanthridin-7-yl)benzenesulfonamide

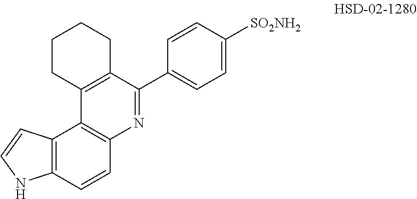 [SMILES: NS(=O)(=O)c1ccc(-c2nc3ccc4[nH]ccc4c3c3c2CCCC3)cc1]

Method A: Off-while solid (162 mg, 43%). ¹H NMR (500 MHz, DMSO-d₆) δ 11.80 (s, 1H), 7.91 (d, J=8.4 Hz, 2H), 7.80 (d, J=8.9 Hz, 1H), 7.73 (d, J=8.4 Hz, 2H), 7.63 (d, J=8.8 Hz, 1H), 7.51 (d, J=3.1 Hz, 1H), 7.39 (s, 2H), 7.14 (d, J=3.1 Hz, 1H), 3.40 (t, J=6.5 Hz, 2H), 2.75 (t, J=6.2 Hz, 2H), 2.03-1.97 (m, 2H), 1.74 (qd, J=8.8, 7.3, 4.2 Hz, 2H); ¹³C NMR (126 MHz, DMSO) δ 154.57, 145.05, 143.70, 143.40, 142.65, 133.35, 130.06, 127.32, 125.76, 124.09, 122.72, 120.30, 116.75, 106.36, 29.96, 28.94, 22.81, 22.46; HRMS (ESI) m/z calcd for C₂₁H₂₀N₃O₂S [M+H]⁺ 378.1276, found 378.1279.

### 7-(1H-Pyrrolo[2,3-b]pyridin-5-yl)-8,9,10,11-tetrahydro-3H-pyrazolo[4,3-a]phenanthridine

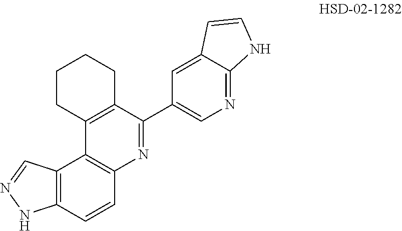 [SMILES: c1cc2cc(-c3nc4ccc5[nH]ncc5c4c4c3CCCC4)cnc2[nH]1]

Method A: Off-white solid (143 mg, 42%). ¹H NMR (500 MHz, DMSO-d₆) δ 11.76 (d, J=2.2 Hz, 1H), 8.56 (s, 1H), 8.41 (d, J=2.1 Hz, 1H), 8.15 (d, J=2.0 Hz, 1H), 7.85 (s, 2H), 7.53 (dd, J=3.4, 2.5 Hz, 1H), 6.51 (dd, J=3.4, 1.8 Hz, 1H), 3.33-3.291 m, 2H), 2.83 (t, J=6.1 Hz, 2H), 2.04-1.95 (m, 2H), 1.79-1.68 (m, 2H); ¹³C NMR (126 MHz, DMSO) δ 155.77, 148.32, 143.79, 142.36, 138.66, 136.36, 129.95, 129.71, 129.03, 128.96, 127.21, 121.73, 119.22, 116.50, 114.34, 100.67, 29.73, 29.23, 22.62; HRMS (ESI) m/z calcd for C₂₁H₁₈N₅ [M+H]⁺ 340.1562, found 340.1565.

### 7-(1-Methyl-1H-indazol-5-yl)-8,9,10,11-tetrahydro-3H-pyrazolo[4,3-a]phenanthridine

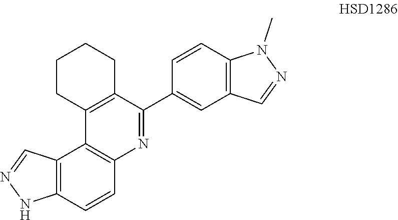 [SMILES: Cn1ncc2cc(-c3nc4ccc5[nH]ncc5c4c4c3CCCC4)ccc21]

Method A: Off-white solid (164 mg, 46%). ¹H NMR (500 MHz, Methanol-d₄) δ 9.13 (s, 1H), 8.84 (s, 1H), 8.26 (d, J=9.2 Hz, 1H), 8.21 (s, 1H), 8.13-8.06 (m, 2H), 7.89 (dd, J=8.5, 1.4 Hz, 1H), 3.63 (t, J=6.4 Hz, 2H), 2.91 (t, J=6.1 Hz, 2H), 2.32-2.11 (m, 2H), 2.05-1.81 (m, 2H); ¹³C NMR (126 MHz, MeOD) δ 153.92, 150.61, 144.58, 135.63, 135.49, 134.39, 132.24, 129.30, 126.03, 123.77, 119.99, 117.90, 114.89, 112.48, 31.75, 30.86, 27.89, 21.33, 21.22; HRMS (ESI) m/z calcd for C₂₂H₂₀N₅ [M+H]⁺ 354.1719, found 354.1721.

### 9-Methyl-7-(3-(trifluoromethyl)-1H-pyrazol-4-yl)-8,9,10,11-tetrahydro-3H-pyrazolo[4,3-a]phenanthridine

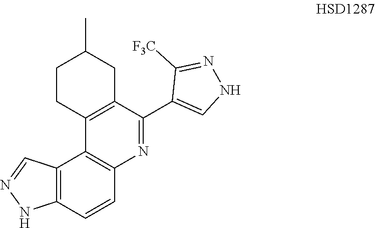 [SMILES: CC1CCc2c(c(-c3c[nH]nc3C(F)(F)F)nc3ccc4[nH]ncc4c23)C1]

Method A: Off-white solid (178 mg, 48%). ¹H NMR (500 MHz, Methanol-d₄) δ 8.60 (s, 1H), 7.97 (d, J=1.1 Hz, 1H), 7.86 (s, 2H), 3.56-3.48 (m, 1H), 3.39-3.32 (m, 2H), 2.77-2.70 (m, 1H), 2.38-2.28 (m, 1H), 2.27-2.18 (m, 1H), 1.97-1.85 (m, 1H), 1.66-1.53 (m, 1H), 1.09 (d, J=6.5 Hz, 3H); ¹³C NMR (126 MHz, MeOD) δ 148.72, 143.06, 142.82, 139.74 (J=28.98 Hz) 134.66, 131.35, 131.12, 131.01, 128.37, 123.01, 122.23, 120.84 (J=269.64 Hz), 115.93, 114.67, 36.13, 30.14, 29.44, 28.15, 20.47; HRMS (ESI) m/z calcd for C₁₈H₁₈N₅ [M+H]⁺ 304.1562, found 304.1565.

### 7-(3-(Trifluoromethyl)-1H-pyrazol-4-yl)-3,8,9,10-tetrahydrocyclopenta[c]pyrazolo[4,3-f]quinoline

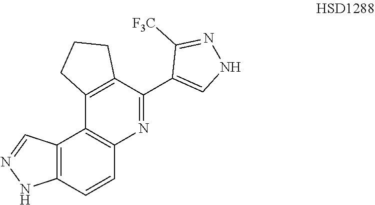 [SMILES: FC(F)(F)c1n[nH]cc1-c1nc2ccc3[nH]ncc3c2c2c1CCC2]

Method A: Off-white solid (185 mg, 54%). ¹H NMR (500 MHz, MeOD) δ 8.76 (s, 1H), 8.44 (s, 1H), 8.33 (d, J=9.3 Hz, 1H), 8.05 (d, J=9.3 Hz, 1H), 3.86 (t, J=7.7 Hz, 2H), 3.21 (t, J=7.7 Hz, 2H), 2.57-2.53 (m, 2H); ¹³C NMR (126 MHz, MeOD) δ 160.67, 141.10, 140.00, 138.86, 136.50, 133.37, 132.59, 122.32 (q, J=269.64 Hz), 121.78, 121.15, 118.59, 115.19, 110.20, 35.03, 30.90, 23.82; HRMS (ESI) m/z calcd for C₁₇H₁₃F₃N₅ [M+H]⁺ 334.1123, found 334.1121.

### 7-(3-(Trifluoromethyl)-1H-pyrazol-4-yl)-3,8,9,10,11,12-hexahydrocyclohepta[c]pyrazolo[4,3-f]quinoline

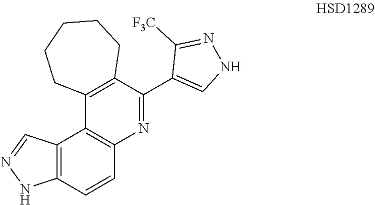 [SMILES: FC(F)(F)c1n[nH]cc1-c1nc2ccc3[nH]ncc3c2c2c1CCCCC2]

Method A: Off-white solid (185 mg, 50%). ¹H NMR (500 MHz, Methanol-d₄) δ 8.98-8.87 (m, 1H), 8.37-8.27 (m, 2H), 7.99 (d, J=9.2 Hz, 1H), 3.86 (d, J=5.3 Hz, 2H), 3.17-3.04 (m, 2H), 2.05 (p, J=2.7 Hz, 4H), 1.78-1.65 (m, 2H); ¹³C NMR (126 MHz, MeOD) δ 160.75, 140.27, 136.43, 133.66, 132.53, 123.83, 122.46, 122.13, 121.07, 120.17 (q, J=269.64 Hz), 118.79, 114.94, 114.65, 110.56, 110.56, 32.30, 30.64, 29.52, 25.85, 23.75; HRMS (ESI) m/z calcd for C₁₉H₁₇F₃N₅ [M+H]⁺ 372.1436, found 372.1438.

### 7-(3-(Trifluoromethyl)-1H-pyrazol-4-yl)-8,9-dihydro-3H-cyclobuta[c]pyrazolo[4,3-f]quinolone

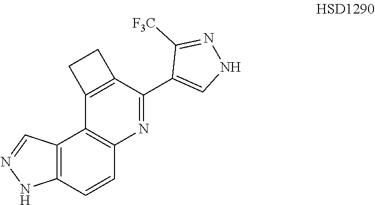 [SMILES: FC(F)(F)c1n[nH]cc1-c1nc2ccc3[nH]ncc3c2c2c1CC2]

Method A: Off-white solid (49 mg, 15%). ¹H NMR (500 MHz, MeOD) δ 8.69 (d, J=0.9 Hz, 1H), 8.53 (d, J=0.9 Hz, 1H), 8.32 (dd, J=9.4, 1.0 Hz, 1H), 8.11 (d, J=9.4 Hz, 1H), 4.01-3.93 (m, 2H), 3.72 (t, J=4.0 Hz, 2H), ¹³C NMR (126 MHz, MeOD) δ 161.67, 140.75, 139.64, 137.73, 136.67, 133.55, 133.12, 122.29 (q, J=268.38 Hz), 121.46, 119.65, 119.42, 113.74, 108.80, 30.70, 29.72; HRMS (ESI) m/z calcd for C₁₆H₁₁F₃N₅ [M+H]⁺ 330.0967, found 330.0970.

### 9-Methyl-7-(3-(trifluoromethyl)-1H-pyrazol-4-yl)-3H-pyrazolo[4,3-f]quinoline

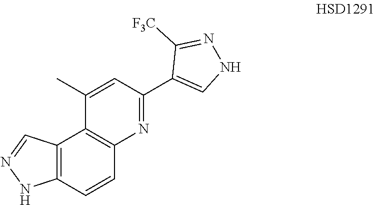 [SMILES: Cc1cc(-c2c[nH]nc2C(F)(F)F)nc2ccc3[nH]ncc3c12]

Method A: Off-white solid (136 mg, 43%). ¹H NMR (500 MHz, MeOD) δ 8.51 (s, 1H), 8.29-8.21 (m, 1H), 7.91-7.81 (m, 2H), 7.62 (s, 1H), 2.89 (s, 3H); ¹³C NMR (126 MHz, MeOD) δ 147.45, 145.74, 144.37, 139.03, 138.40, 135.27, 130.97, 128.87, 125.13 (J=269.64 Hz), 122.50, 121.51, 120.86, 116.35, 114.74, 21.45; HRMS (ESI) m/z calcd for C₁₅H₁₁F₃N₅ [M+H]⁺ 318.0967, found 318.0971.

### 7-(1H-1,2,3-Triazol-4-yl)-8,9,10,11-tetrahydro-3H-pyrazolo[4,3-a]phenanthridine

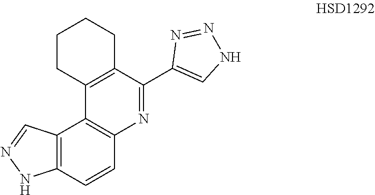 [SMILES: c1[nH]nnc1-c1nc2ccc3[nH]ncc3c2c2c1CCCC2]

Method A: Off-white solid (159 mg, 55%). ¹H NMR (500 MHz, DMSO-d₆) δ 8.57 (s, 1H), 8.31 (d, J=1.9 Hz, 1H), 7.87 (d, J=9.2 Hz, 1H), 7.82 (dd, J=9.1, 1.9 Hz, 1H), 3.30 (d, J=6.6 Hz, 2H), 3.12 (d, J=6.4 Hz, 2H), 2.05-1.94 (m, 2H), 1.84 (dtd, J=10.1, 6.9, 6.3, 3.3 Hz, 2H); ¹³C NMR (126 MHz, DMSO) δ 146.46, 143.65, 142.93, 131.52, 129.80, 129.48, 122.18, 116.16, 115.49, 29.87, 28.27, 22.37; HRMS (ESI) m/z calcd for C₁₆H₁₅N₆ [M+H]⁺ 291.1358, found 291.1360.

### Methyl 3-(8,9,10,11-tetrahydro-3H-pyrazolo[4,3-a]phenanthridin-7-yl)-1H-pyrrole-2-carboxylate

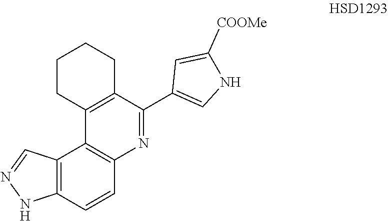 [SMILES: COC(=O)c1cc(-c2nc3ccc4[nH]ncc4c3c3c2CCCC3)c[nH]1]

Method A: Off-white solid (142 mg, 41%). ¹H NMR (500 MHz, DMSO-d₆) δ 12.20 (s, 1H), 8.50 (s, 1H), 7.52 (s, 1H), 7.34 (s, 1H), 3.80 (s, 3H), 3.32-3.24 (m, 2H), 3.01 (t, J=6.1 Hz, 2H), 2.03-1.91 (m, 2H), 1.88-1.74 (m, 2H); ¹³C NMR (126 MHz, DMSO) δ 161.40, 150.78, 143.78, 142.06, 138.38, 136.17, 136.16, 129.69, 129.00, 125.75, 125.46, 122.08, 121.04, 116.53, 114.14, 51.67, 29.86, 28.94, 22.68, 22.50; HRMS (ESI) m/z calcd for C₂₀H₁₉N₄O₂ [M+H]⁺ 347.1508, found 347.1513.

### 2-(4-(8,9,10,11-Tetrahydro-3H-pyrazolo[4,3-a]phenanthridin-7-yl)-1H-pyrazol-1-yl)ethan-1-ol

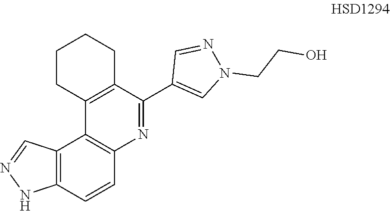 [SMILES: OCCn1cc(-c2nc3ccc4[nH]ncc4c3c3c2CCCC3)cn1]

Method A: Off-white solid (126 mg, 38%). ¹H NMR (500 MHz, Methanol-d₄) δ 8.51 (s, 1H), 8.12 (s, 1H), 7.99 (s, 1H), 7.87 (d, J=9.2 Hz, 1H), 7.79 (d, J=9.0 Hz, 1H), 4.34 (t, J=5.4 Hz, 2H), 3.97 (t, J=5.4 Hz, 2H), 3.35-3.32 (m, 2H), 3.00 (t, J=6.2 Hz, 2H), 2.10-2.03 (m, 2H), 1.95-1.86 (m, 2H), ¹³C NMR (126 MHz, MeOD) δ 149.36, 143.28, 143.07, 139.58, 138.49, 135.72, 131.37, 129.45, 128.70, 121.71, 121.43, 116.16, 113.49, 60.48, 54.17, 29.73, 28.38, 22.12, 22.03; HRMS (ESI) m/z calcd for C₁₉H₂₀N₅O [M+H]⁺ 334.1668, found 334.1675.

### 7-(1,3-Dimethyl-1H-pyrazol-4-yl)-8,9,10,11-tetrahydro-3H-pyrazolo[4,3-a]phenanthridine

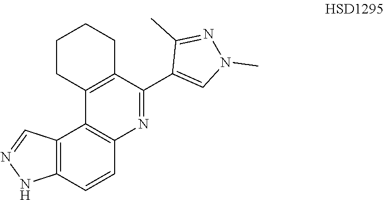 [SMILES: Cc1nn(C)cc1-c1nc2ccc3[nH]ncc3c2c2c1CCCC2]

Method A: Off-white solid (180 mg, 57%). ¹H NMR (500 MHz, DMSO-d₆) δ 8.53 (s, 1H), 7.92 (s, 1H), 7.81 (d, J=9.0 Hz, 1H), 7.76 (d, J=9.1 Hz, 1H), 3.82 (s, 3H), 3.27 (t, J=6.5 Hz, 3H), 2.81 (t, J=6.2 Hz, 2H), 2.26 (s, 3H), 2.01-1.88 (m, 2H), 1.82-1.67 (m, 2H); ¹³C NMR (126 MHz, DMSO) δ 150.70, 147.24, 143.71, 141.82, 139.32, 135.07, 131.72, 129.87, 129.57, 121.05, 118.85, 116.35, 114.88, 38.70, 29.70, 28.60, 22.60, 13.53; HRMS (ESI) m/z calcd for C₁₉H₂₀N₅ [M+H]⁺ 318.1719, found 318.1725.

### 7-(1H-Imidazol-4-yl)-8,9,10,11-tetrahydro-3H-pyrazolo[4,3-a]phenanthridine

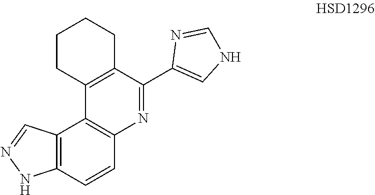 [SMILES: c1nc(-c2nc3ccc4[nH]ncc4c3c3c2CCCC3)c[nH]1]

Method A: Off-white solid (168 mg, 58%). ¹H NMR (500 MHz, DMSO-d₆) δ 8.62-8.53 (m, 2H), 7.93-7.82 (m, 3H), 3.36-3.23 (m, 2H), 3.09-2.97 (m, 2H), 2.02-1.94 (m, 2H), 1.90-1.78 (m, 2H); ¹³C NMR (126 MHz, DMSO) δ 144.79, 143.75, 142.88, 138.70, 136.05, 132.80, 131.40, 129.06, 128.65, 122.65, 121.92, 116.12, 115.25, 29.94, 27.98, 22.22; HRMS (ESI) m/z calcd for C₁₇H₁₆N₅ [M+H]⁺ 290.1406, found 290.1415.

### 1-Iodo-7-(3-(trifluoromethyl)-1H-pyrazol-4-yl)-8,9,10,11-tetrahydro-3H-pyrazolo[4,3-a]phenanthridine

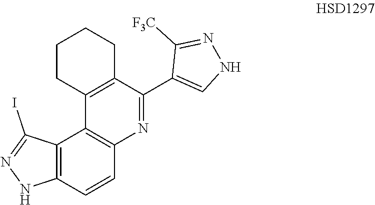 [SMILES: FC(F)(F)c1n[nH]cc1-c1nc2ccc3[nH]nc(I)c3c2c2c1CCCC2]

Method A: Off-white solid (145 mg, 30%). ¹H NMR (500 MHz, Methanol-d₄) δ 8.90 (s, 1H), 8.35 (s, 1H), 8.33 (d, J=9.1 Hz, 1H), 8.00 (d, J=9.2 Hz, 1H), 3.67 (t, J=6.4 Hz, 2H), 2.81 (t, J=6.2 Hz, 2H), 2.18 (qd, J=7.8, 6.3, 4.5 Hz, 2H), 2.04-1.90 (m, 2H); ¹³C NMR (126 MHz, MeOD) δ 154.90, 142.03, 140.54, 139.83, 134.96, 134.44, 134.02, 132.40, 124.40, 122.30 (J=269.64 Hz), 121.60, 118.81, 114.76, 109.91, 30.80, 27.22, 21.20, 20.87; HRMS (ESI) m/z calcd for C₁₇H₁₆N₅ [M+H]⁺ 484.0246, found 484.0252.

### 7-(3-Phenyl-1H-pyrazol-4-yl)-8,9,10,11-tetrahydro-3H-pyrazolo[4,3-a]phenanthridine

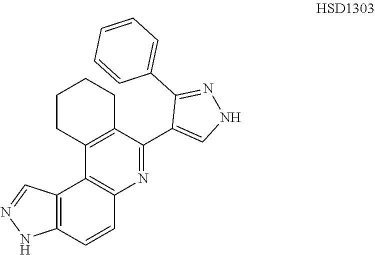 [SMILES: c1ccc(-c2n[nH]cc2-c2nc3ccc4[nH]ncc4c3c3c2CCCC3)cc1]

Method A: Off-white solid (168 mg, 45%). ¹H NMR (500 MHz, DMSO-d₆) δ 8.81 (s, 1H), 8.27 (d, J=9.1 Hz, 2H), 8.17 (d, J=9.2 Hz, 1H), 7.35-7.22 (m, 5H), 3.51-3.43 (m, 2H), 2.55-2.49 (m, 0H), 1.95-1.82 (m, 2H), 1.72-1.61 (m, 2H); ¹³C NMR (126 MHz, DMSO) δ 145.52, 135.52, 132.94, 129.41, 128.96, 128.85, 128.19, 126.99, 123.39, 120.03, 114.91, 109.66, 30.80, 27.32, 21.75, 21.27; HRMS (ESI) m/z calcd for C23H20N5 [M+H]⁺ 366.1719, found 366.1726.

### 7-(3-(Trifluoromethyl)-1H-pyrazol-4-yl)-8,9,10,11-tetrahydro-3H-pyrazolo[4,3-a]phenanthridin-9-amine

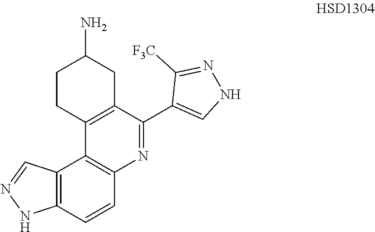 [SMILES: NC1CCc2c(c(-c3c[nH]nc3C(F)(F)F)nc3ccc4[nH]ncc4c23)C1]

Method A: Off-white solid (137 mg, 37%). ¹H NMR (500 MHz, Methanol-d₄) δ 8.57 (d, J=7.7 Hz, 1H), 8.03-8.00 (m, 1H), 7.87-7.83 (m, 2H), 3.92-3.82 (m, 1H), 3.65-3.53 (m, 1H), 3.49-3.38 (m, 1H), 2.81-2.65 (m, 2H), 2.25-2.17 (m, 1H), 2.08-1.96 (m, 1H); ¹³C NMR (126 MHz, MeOD) δ 148.28, 143.27, 142.44, 142.22, 139.73, 134.64, 130.59, 129.31, 128.37, 122.83 (q, J=269.64 Hz), 122.00, 118.74, 115.86, 115.82, 114.97, 46.06, 36.25, 30.30, 28.58; HRMS (ESI) m/z calcd for C₁₆H₁₆F₃N₆ [M+H]⁺ 373.1389, found 373.1393.

### 3-Methyl-3-(4-(8,9,10,11-tetrahydro-3H-pyrazolo[4,3-a]phenanthridin-7-yl)-1H-pyrazol-1-yl)tetrahydrothiophene 1,1-dioxide

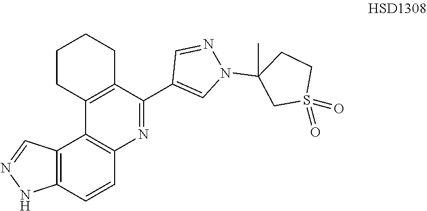 [SMILES: CC1(n2cc(-c3nc4ccc5[nH]ncc5c4c4c3CCCC4)cn2)CCS(=O)(=O)C1]

Method A: Off-white solid (126 mg, 30%). ¹H NMR (500 MHz, DMSO-d₆) δ 8.54-8.48 (m, 1H), 8.47 (s, 1H), 8.08 (s, 1H), 7.81 (s, 2H), 4.22 (dd, J=14.1, 1.7 Hz, 1H), 3.59 (d, J=14.1 Hz, 1H), 3.42 (ddd, J=12.7, 7.7, 4.6 Hz, 1H), 3.29 (d, J=6.5 Hz, 2H), 3.19 (ddd, J=13.1, 9.6, 7.5 Hz, 1H), 3.15-3.07 (m, 1H), 2.55 (ddd, J=13.9, 9.6, 7.7 Hz, 1H), 1.98 (ddt, J=12.1, 6.1, 3.3 Hz, 2H), 1.84 (qd, J=9.1, 7.4, 4.0 Hz, 2H), 1.75 (s, 3H); ¹³C NMR (126 MHz, DMSO) δ 149.12, 143.66, 142.26, 140.19, 138.52, 136.20, 129.53, 129.02, 128.80, 123.04, 121.18, 116.52, 114.29, 65.32, 61.46, 51.53, 35.09, 29.87, 28.53, 27.91, 22.63, 22.50.

### 7-(3-(Pyridin-3-yl)-1H-pyrazol-4-yl)-8,9,10,11-tetrahydro-3H-pyrazolo[4,3-a]phenanthridine

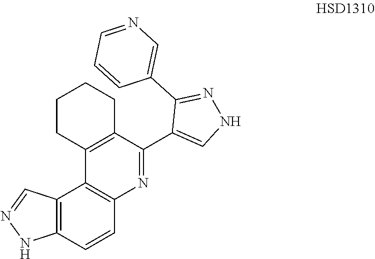 [SMILES: c1cncc(-c2n[nH]cc2-c2nc3ccc4[nH]ncc4c3c3c2CCCC3)c1]

Method A: Off-white solid (172 mg, 47%). ¹H NMR (500 MHz, DMSO-d₆) δ 8.56 (d, J=1.0 Hz, 1H), 8.49 (dd, J=2.2, 0.9 Hz, 1H), 8.36 (dd, J=4.7, 1.7 Hz, 1H), 7.98 (s, 1H), 7.83 (dd, J=9.0, 0.9 Hz, 1H), 7.72 (d, J=8.0, 2.3, 1.7 Hz, 1H), 7.68 (d, J=9.0 Hz, 1H), 7.25 (ddd, J=8.0, 4.8, 0.9 Hz, 1H), 3.29 (t, J=6.5 Hz, 2H), 2.58 (t, J=6.2 Hz, 2H), 1.89 (ddt, J=9.2, 6.4, 4.2 Hz, 2H), 1.73-1.64 (m, 2H); ¹³C NMR (126 MHz, DMSO) δ 150.81, 148.62, 147.71, 143.91, 143.51, 142.22, 140.11, 135.03, 134.04, 133.41, 130.51, 129.58, 129.11, 123.91, 121.95, 119.13, 116.29, 115.55, 29.57, 28.27, 22.59, 22.35; HRMS (ESI) m/z calcd for C₂₂H₁₉N₆[M+H]⁺ 367.1671, found 367.1675.

### 7-(1-Phenyl-1H-pyrazol-4-yl)-8,9,10,11-tetrahydro-3H-pyrazolo[4,3-a]phenanthridine

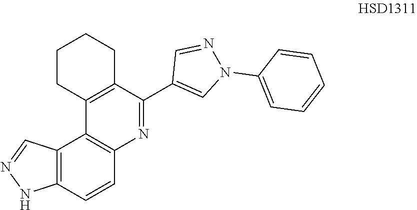 [SMILES: c1ccc(-n2cc(-c3nc4ccc5[nH]ncc5c4c4c3CCCC4)cn2)cc1]

Method A: Off-white solid (186 mg, 51%). ¹H NMR (500 MHz, DMSO-d₆) δ 8.55 (s, 1H), 7.95 (s, 1H), 7.88-7.73 (m, 2H), 7.37-7.23 (m, 1H), 6.79 (s, 1H), 6.63 (s, 1H), 3.31 (t, J=6.4 Hz, 2H), 2.58 (t, J=6.1 Hz, 2H), 1.94-1.85 (m, 2H), 1.74-1.65 (m, 2H); ¹³C NMR (126 MHz, DMSO) δ 150.93, 143.68, 142.18, 138.73, 136.97, 136.67, 136.31, 131.22, 130.28, 129.55, 127.67, 125.20, 123.94, 122.00, 118.02, 116.54, 114.31, 29.56, 28.10, 22.55, 22.32; HRMS (ESI) m/z calcd for C₂₃H₂₀N₅ [M+H]⁺ 366.1719, found 366.1720.

### 7-(3-(Thiophen-3-yl)-1H-pyrazol-4-yl)-8,9,10,11-tetrahydro-3H-pyrazolo[4,3-a]phenanthridine

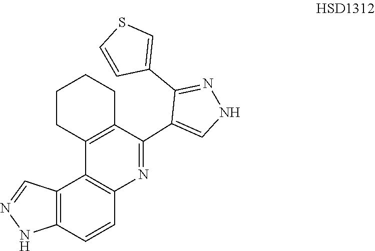 [SMILES: c1cc(-c2n[nH]cc2-c2nc3ccc4[nH]ncc4c3c3c2CCCC3)cs1]

Method A: Off-white solid (177 mg, 46%). ¹H NMR (500 MHz, DMSO-d₆) δ 8.89 (d, J=1.8 Hz, 1H), 8.55 (s, 1H), 8.29 (d, J=1.7 Hz, 1H), 7.95 (d, J=8.2 Hz, 2H), 7.83 (s, 2H), 7.57-7.46 (m, 2H), 7.39-7.28 (m, 1H), 3.29 (d, J=6.5 Hz, 2H), 3.07 (t, J=6.2 Hz, 2H), 2.03-1.94 (m, 2H), 1.86 (tt, J=9.6, 5.4 Hz, 2H); ¹³C NMR (126 MHz, DMSO) δ 148.63, 143.82, 142.45, 142.18, 141.18, 140.01, 139.07, 130.03, 129.57, 129.19, 127.95, 126.90, 124.53, 121.44, 119.01, 118.46, 116.29, 29.86, 28.53, 22.57, 22.45; HRMS (ESI) m/z calcd for C₂₁H₁₈N₅S [M+H]⁺ 372.1283. found 372.1286.

### 7-(3-(tert-Butyl)-1H-pyrazol-4-yl)-8,9,10,11-tetrahydro-3H-pyrazolo[4,3-a]phenanthridine

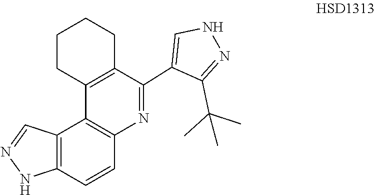 [SMILES: CC(C)(C)c1n[nH]cc1-c1nc2ccc3[nH]ncc3c2c2c1CCCC2]

Method A: Off-white solid (176 mg, 51%). ¹H NMR (500 MHz, MeOD) δ 8.55 (s, 1H), 7.88-7.79 (m, 2H), 7.53 (s, 1H), 3.37-3.31 (m, 2H), 2.67 (t, J=6.2 Hz, 2H), 2.08-1.97 (m, 2H), 1.90-1.81 (m, 2H); ¹³C NMR (126 MHz, MeOD) δ 152.31, 143.08, 142.67, 138.68, 135.79, 131.90, 128.29, 122.19, 116.70, 116.13, 113.68, 32.29, 32.24, 29.44, 29.30, 28.47, 22.15, 21.89; HRMS (ESI) m/z calcd for C₂₁H₂₄N₅ [M+H]⁺ 346.2032, found 346.2021.

### 4-(8,9,10,11-Tetrahydro-3H-pyrazolo[4,3-a]phenanthridin-7-yl)-1H-pyrazole-3-carboxylic acid

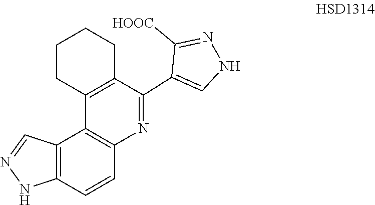 [SMILES: O=C(O)c1n[nH]cc1-c1nc2ccc3[nH]ncc3c2c2c1CCCC2]

Method A: Off-white solid (167 mg, 50%). ¹H NMR (500 MHz, DMSO-d₆) δ NH 8.62 (s, 1H), 8.25 (s, 1H), 7.99 (d, J=9.1 Hz, 1H), 7.76 (d, J=9.4 Hz, 1H), 3.30 (t, J=6.5 Hz, 2H), 2.93-2.78 (m, 2H), 2.03-1.91 (m, 2H), 1.83-1.74 (m, 2H); ¹³C NMR (126 MHz, DMSO) δ 162.03, 148.76, 145.57, 139.73, 137.07, 136.25, 131.58, 128.69, 123.61, 122.18, 121.89, 116.71, 115.13, 30.40, 28.34, 21.77. HRMS (ESI) m/z calcd for C₁₈H₁₆N₅O₂ [M+H]⁺ 334.1304, found 334.1308.

### 7-(1-(2-Chloroethyl)-1H-pyrazol-4-yl)-8,9,10,11-tetrahydro-3H-pyrazolo[4,3-a]phenanthridine

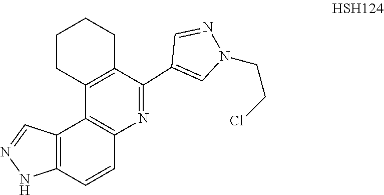 [SMILES: ClCCn1cc(-c2nc3ccc4[nH]ncc4c3c3c2CCCC3)cn1]

Method A: Off-white solid Yield: (26 mg, 28%) ¹H NMR (500 MHz, Methanol-d₄) δ 8.83 (s, 1H), 8.55 (d, J=0.8 Hz, 1H), 8.29-8.20 (m, 2H), 8.12 (d, J=9.2 Hz, 1H), 4.68 (t, J=5.6 Hz, 2H), 4.11-4.04 (m, 2H), 3.62 (t, J=6.4 Hz, 2H), 3.13 (t, J=6.2 Hz, 2H), 2.25-2.15 (m, 2H), 2.07-1.97 (m, 2H); ¹³C NMR (126 MHz, Methanol-d₄) δ 154.27, 143.82, 140.54, 134.63, 133.72, 131.88, 122.97, 118.99, 114.92, 112.57, 53.74, 42.44, 31.04, 27.80, 21.23.

### 2,3-Dimethyl-7-(3-(trifluoromethyl)-1H-pyrazol-4-yl)-3,6,8,9,10,11-hexahydro-2H-pyrazolo[3,4-a]phenanthridine

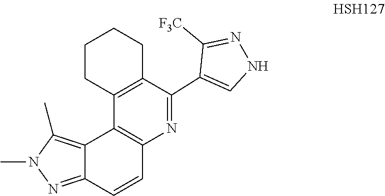 [SMILES: Cc1c2c(ccc3nc(-c4c[nH]nc4C(F)(F)F)c4c(c32)CCCC4)nn1C]

Method A Off-white solid, Yield: (40 mg, 20%) ¹H NMR (500 MHz, Methanol-d₄) δ 8.33 (s, 1H), 8.27 (d, J=9.0 Hz, 1H), 7.53 (d, J=9.0 Hz, 1H), 4.27 (s, 3H), 3.95 (t, J=6.4 Hz, 2H), 2.79-2.76 (m, 5H), 2.13-2.05 (m, 2H), 1.98-1.86 (m, 2H); ¹³C NMR (126 MHz, Methanol-d₄) δ 156.81, 142.12, 141.73, 138.79, 135.59, 132.97, 132.27, 129.07, 122.32 (q, J=268), 121.35, 119.06, 111.69, 110.05, 36.88, 31.69, 27.09, 21.21, 20.94, 8.19.

### 5-Fluoro-7-(3-(trifluoromethyl)-1H-pyrazol-4-yl)-8,9,10,11-tetrahydro-3H-pyrazolo[4,3-a]phenanthridine

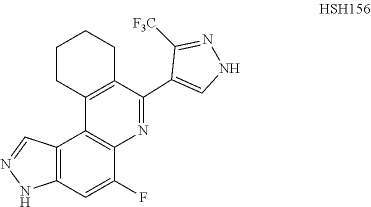 [SMILES: Fc1cc2[nH]ncc2c2c3c(c(-c4c[nH]nc4C(F)(F)F)nc12)CCCC3]

Method A White solid. Yield: (94 mg, 25%) ¹H NMR (500 MHz, DMSO-d₆) δ 8.60 (s, 1H), 8.25 (s, 1H), 7.69 (d, J=9.9 Hz, 1H), 3.29 (t, J=6.4 Hz, 2H), 2.71 (t, J=6.2 Hz, 2H), 1.95 (dt, J=12.3, 4.6 Hz, 2H), 1.87-1.71 (m, 2H); ¹³C NMR (126 MHz, DMSO-d₆) δ 158.10, 156.09, 148.56, 142.63, 134.68, 134.59, 131.84, 131.42, 123.43 (q, J=268), 122.90, 119.33, 112.71, 29.54, 28.19, 22.40, 22.13. HRMS (ESI) m/z calcd for C₁₈H₁₄F₄N₅ [M+H]⁺ 376.1185, found 376.1185.

### 7-(3-(Trifluoromethyl)-1H-pyrazol-4-yl)-8,9,10,11-tetrahydro-3H-pyrazolo[4,3-a]phenanthridin-5-ol

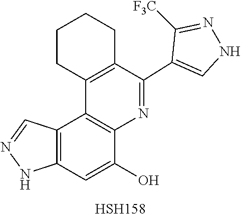

Method A Off-white solid, Yield: (21 mg, 11%) ¹H NMR (500 MHz, Methanol-d₄) δ 9.05 (s, 1H), 8.35 (t, J=1.0 Hz, 1H), 8.19 (s, 1H), 3.63 (t, J=6.4 Hz, 2H), 2.79 (t, J=6.2 Hz, 2H), 2.24-2.14 (m, 2H), 2.06-1.90 (m, 2H); ¹³C NMR (126 MHz, Methanol-d₄) δ 154.95, 142.50, 139.83, 135.25, 134.31, 133.85, 132.44, 123.87, 122.28 (q, J=269.64), 120.97, 115.06, 109.89, 30.71, 27.27, 21.14, 20.80.

### 5-Bromo-7-(3-(trifluoromethyl)-1H-pyrazol-4-yl)-8,9,10,11-tetrahydro-3H-pyrazolo[4,3-a]phenanthridine

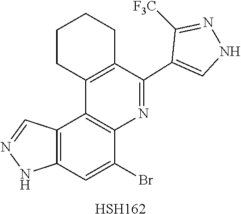

Method A Off-white solid, Yield: (10 mg, 2%) ¹H NMR (500 MHz, DMSO-d₆) δ 8.61 (s, 1H), 8.29 (s, 2H), 3.32-3.28 (m, 2H), 2.75 (t, J=6.2 Hz, 2H), 1.96 (m, 2H), 1.78 (m, 2H); ¹³C NMR (126 MHz, DMSO-d₆) δ 148.91, 143.00, 139.74, 139.73, 139.70, 131.56, 131.54, 131.51, 124.67, 123.42, 122.68, 121.27 (q, J=270.9 Hz), 119.30, 55.37, 29.71, 28.24, 22.49, 22.17.

### 5-Chloro-7-(3-(trifluoromethyl)-1H-pyrazol4-yl)-8,9,10,11-tetrahydro-3H-pyrazolo[4,3-a]phenanthridine

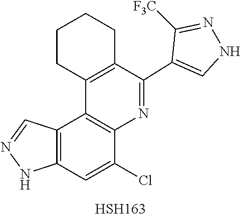

Method A Off-white solid. Yield: (12 mg, 5%)¹H NMR (500 MHz, DMSO-d₆) δ 8.62 (s, 1H), 8.29 (s, 1H), 8.09 (s, 1H), 3.31-3.26 (m, 2H), 2.75 (t, J=6.2 Hz, 2H), 2.06-1.87 (m, 2H), 1.82-1.67 (m, 2H); ¹³C NMR (126 MHz, DMSO-d₆) δ 148.69, 143.00, 139.26, 132.77, 131.55, 131.55, 129.57, 123.42 (q, J=269), 122.85, 119.37, 119.36, 115.32, 29.72, 28.23, 22.48, 22.16.

### 4-Methyl-7-(3-(trifluoromethyl)-1H-pyrazol-4-yl)-8,9,10,11-tetrahydro-3H-pyrazolo[4,3-a]phenanthridine

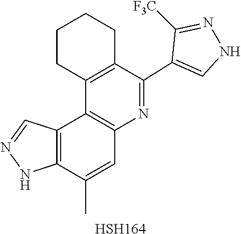

Method A Off-white solid, Yield: (20 mg, 5%). ¹H NMR (500 MHz, DMSO-d₆) δ 8.55 (s, 1H), 8.20 (s, 1H), 7.56 (s, 1H), 3.32-3.30 (m, 2H), 2.68 (t, J=6.2 Hz, 2H), 2.63 (s, 3H), 1.96 (m, 2H), 1.77 (m, 2H); ¹³C NMR (126 MHz, DMSO-d₆) δ 148.32, 144.01, 141.68, 139.48, 136.72, 131.12, 129.51, 128.11, 124.44, 123.55 (q, J=269), 121.41, 119.83, 116.19, 29.44, 28.13, 22.61, 22.41, 17.46.

### 7-(3-(Trifluoromethyl)-1H-pyrazol-4-yl)-8,9,10,11-tetrahydro-3[1,2,3]triazolo[4,5-a]phenanthridine

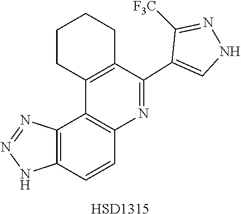

Method A Off-white solid, Yield: (72 mg, 20%). ¹H NMR (500 MHz, Methanol-d₄) δ 8.40 (d, J=9.2 Hz, 1H), 8.33 (s, 1H), 8.05 (d, J=9.2 Hz, 1H), 4.01 (t, J=6.4 Hz, 2H), 2.82 (t, J=6.3 Hz, H), 2.13 (dp, J=9.3, 3.1 Hz, 2H), 2.02-1.93 (m, 2H); ¹³C NMR (126 MHz, MeOD) δ 154.39, 145.35, 140.07, 139.75, 138.56, 137.77, 134.33, 132.14, 122.37 (q, J=269.6 Hz), 121.20, 120.95, 116.39, 111.31, 30.87, 27.26, 21.12, 21.07; HRMS (ESI) m/z calcd for C₁₇H₁₄F₃N₆ [M+H]⁺ 359.1232, found 359.1239.

### 7-(3-(Trifluoromethyl)-1H-pyrazol-4-yl)-8,9,10,11-tetrahydroisoxazolo[4,5-a]phenanthridine

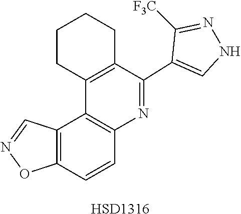

Method A: Off-white solid (126 mg, 35%). ¹H NMR (500 MHz, Methanol-d₄) δ 9.70-9.59 (m, 1H), 8.34-8.22 (m, 3H), 3.62 (t, J=6.4 Hz, 2H), 2.79 (t, J=6.3 Hz, 2H), 2.19-2.08 (m, 2H), 2.00-1.86 (m, 2H). HRMS (ESI) m/z calcd for C₁₈H₁₄F₃N₄O [M+H]⁺ 359.1120, found 359.1122.

### 7-(3-(Trifluoromethyl)-1H-pyrazol-4-yl)-8,9,10,11-tetrahydroisothiazolo[4,5-a]phenanthridine

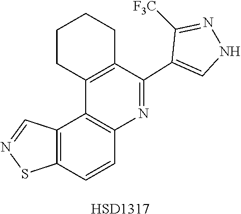

Method A: Off-white solid (120 mg, 32%). ¹H NMR (500 MHz, Methanol-d₄) δ 9.94 (s, 1H), 8.78 (d, J=8.9 Hz, 1H), 8.41 (s, 1H), 8.21 (d, J=8.9 Hz, 1H), 3.85 (t, J=6.1 Hz, 2H), 2.87 (t, J=6.1 Hz, 2H), 2.24-2.13 (m, 2H), 2.00 (td, J=11.2, 9.5, 5.6 Hz, 2H); ¹³C NMR (126 MHz, MeOD) δ 156.17, 155.87, 155.79, 145.11, 140.04, 139.75, 136.42, 135.01, 132.65, 129.80, 125.95, 125.43, 121.58, 120.19, 118.46, 109.79, 32.51, 27.76, 21.37, 20.63; HRMS (ESI) m/z calcd for C₁₈H₁₄F₃N₄S [M+H]⁺ 375.0891, found 375.0899.

### 3-Methyl-7-(3-(trifluoromethyl)-1H-pyrazol-4-yl)-8,9,10,11-tetrahydro-3H-pyrazolo[4,3-a]phenanthridine

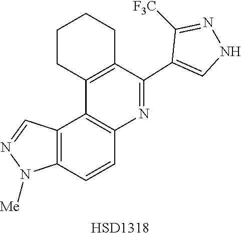 [SMILES: Cn1ncc2c3c4c(c(-c5c[nH]nc5C(F)(F)F)nc3ccc21)CCCC4 ; "HSD1318"]

Method A: Off-white solid (148 mg, 40%). ¹H NMR (500 MHz, Methanol-d₄) δ 8.77 (d, J=0.9 Hz, 1H), 8.42 (dd, J=9.3, 0.9 Hz, 1H), 8.38 (d, J=0.9 Hz, 1H), 8.07 (d, J=9.3 Hz, 1H), 4.32 (s, 3H), 3.68 (t, J=6.3 Hz, 2H), 2.82 (t, J=6.2 Hz, 2H), 2.22-2.12 (m, 2H), 2.05-1.92 (m, 2H); ¹³C NMR (126 MHz, MeOD) δ 155.03, 142.35, 140.13 (q, J=37.8 Hz), 138.47, 135.21, 134.39, 134.19, 132.52, 124.04, 122.33 (q, J=269.6 Hz), 119.30, 118.65, 115.97, 109.85, 35.32, 30.84, 27.26, 21.19, 20.86; HRMS (ESI) m/z calcd for C₁₉H₁₇F₃N₅[M+H]⁺ 372.1436, found 372.1438.

### 6-(5-Fluoro-8,9,10,11-tetrahydro-3H-pyrazolo[4,3-a]phenanthridin-7-yl)benzo[d]thiazol-2-amine

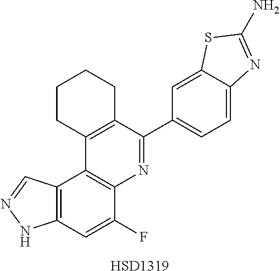

Method A: Off-white solid (148 mg, 38%). ¹H NMR (500 MHz, DMSO-d₆) δ 8.56 (s, 1H), 7.91-7.84 (m, 1H), 7.70 (d, J=9.8 Hz, 1H), 7.57 (d, J=2.4 Hz, 2H), 7.45-7.38 (m, 2H), 3.36-3.32 (m, 2H), 2.84 (t, J=6.1 Hz, 2H), 2.04-1.97 (m, 2H), 1.79-1.71 (m, 2H); ¹³C NMR (126 MHz, DMSO) δ 167.68, 158.36 (d, J=253.26 Hz), 157.08, 153.14, 142.83, 136.79, 136.49, 134.35, 133.64, 131.24, 131.14, 127.29, 122.53, 122.22, 117.37, 113.06, 98.20, 29.80, 29.24, 22.52, 22.44; HRMS (ESI) m/z calcd for C₂₁H₁₇FN₅S [M+H]⁺ 390.1189, found 390.1195.

### 6-(8,9,10,11-Tetrahydro-3H-[1,2,3]triazolo[4,5-a]phenanthridin-7-yl)benzo[d]thiazol-2-amine

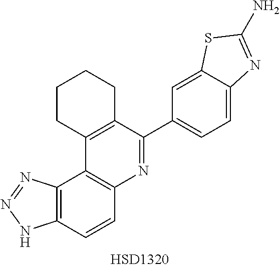 [SMILES: Nc1nc2ccc(-c3nc4ccc5[nH]nnc5c4c4c3CCCC4)cc2s1]

Method A: Off-white solid (112 mg, 30%). ¹H NMR (500 MHz, DMSO-d₆) δ 8.03 (d, J=9.1 Hz, 1H), 7.92-7.88 (m, 2H), 7.57 (s, 2H), 7.44 (dd, J=8.2, 1.7 Hz, 1H), 7.40 (d, J=8.2 Hz, 1H), 3.69 (t, J=6.5 Hz, 2H), 2.85 (t, J=6.2 Hz, 2H), 2.02-1.98 (m, 2H), 1.79-1.73 (m, 2H); ¹³C NMR (126 MHz, DMSO) δ 167.75, 158.87, 153.15, 145.33, 143.19, 139.18, 133.64, 131.24, 130.36, 130.18, 127.24, 122.15, 118.50, 117.37, 116.27, 29.93, 29.07, 22.66, 22.39. HRMS (ESI) m/z calcd for C₂₀H₁₇N₆S [M+H]⁺ 373.1235, found 373.1237.

### 7-(3-Isopropyl-1H-pyrazol-4-yl)-8,9,10,11-tetrahydro-3H-pyrazolo[4,3-a]phenanthridine

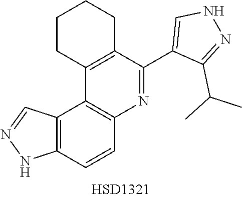 [SMILES: CC(C)c1n[nH]cc1-c1nc2ccc3[nH]ncc3c2c2c1CCCC2]

Method A: Off-white solid (136 mg, 41%). ¹H NMR (500 MHz, DMSO-d₆) δ 8.82 (s, 1H), 8.23 (d, J=14.6 Hz, 2H), 7.97 (s, 1H), 3.49 (t, J=6.2 Hz, 2H), 3.02 (dd, J=14.1, 7.3 Hz, 1H), 2.84-2.66 (m, 2H), 2.20-1.96 (m, 2H), 1.91-1.76 (m, 2H), 1.26-1.08 (m, 6H). HRMS (ESI) m/z calcd for C₂₀H₂₂N₅ [M+H]⁺ 332.1875, found 332.1875.

### 4-(7-(3-(Trifluoromethyl)-1H-pyrazol-4-yl)-8,9,10,11-tetrahydro-3H-pyrazolo[4,3-a]phenanthridin-9-yl)morpholine

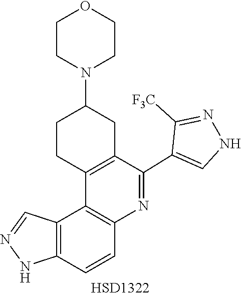 [SMILES: FC(F)(F)c1n[nH]cc1-c1nc2ccc3[nH]ncc3c2c2c1CC(N1CCOCC1)CC2]

Method A: Off-white solid (110 mg, 25%). ¹H NMR (500 MHz, Methanol-d₄) δ 8.64 (s, 1H), 8.13 (s, 1H), 7.95 (d, J=9.2 Hz, 1H), 7.90 (d, J=9.2 Hz, 1H), 4.17-3.37 (m, 11H), 3.29-3.19 (m, 1H), 3.19-3.08 (m, 1H), 2.85-2.70 (m, 1H), 2.14 (qd, J=12.1, 5.8 Hz, 1H). HRMS (ESI) m/z calcd for C₂₂H₂₂F₃N₆O [M+H]⁺ 443.1807, found 443.1809.

### 6-(3-Methyl-8,9,10,11-tetrahydro-3H-pyrazolo[4,3-a]phenanthridin-7-yl)benzo[d]thiazol-2-amine

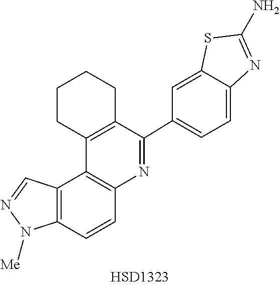 [SMILES: Cn1ncc2c3c4c(c(-c5ccc6nc(N)sc6c5)nc3ccc21)CCCC4]

Method A: Off-white solid (138 mg, 36%). ¹H NMR (500 MHz, DMSO-d₆) δ 8.50 (d, J=0.9 Hz, 1H), 8.02-7.95 (n, 1H), 7.91-7.85 (m, 2H), 7.63 (s, 2H), 7.46-7.36 (m, 2H), 4.17 (s, 3H), 3.31 (t, J=6.5 Hz, 2H), 2.82 (t, J=6.1 Hz, 2H), 2.01-1.95 (m, 2H), 1.83-1.66 (m, 2H); ¹³C NMR (126 MHz, DMSO) δ 167.75, 156.84, 152.94, 143.09, 142.79, 138.18, 135.06, 133.62, 131.17, 129.90, 129.18, 127.28, 122.20, 121.53, 117.29, 117.17, 114.03, 36.31, 29.76, 29.17, 22.58, 22.54. HRMS (ESI) m/z calcd for C₂₂H₂₀N₅S [M+H]⁺ 386.1439, found 386.1442.

### Methyl 7-(2-aminobenzo[d]thiazol-6-yl)-8,9,10,11-tetrahydro-3H-pyrazolo[4,3-a]phenanthridine-1-carboxylate

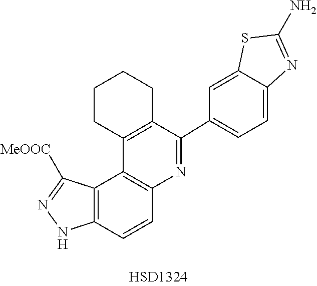 [SMILES: COC(=O)c1n[nH]c2ccc3nc(-c4ccc5nc(N)sc5c4)c4c(c3c12)CCCC4]

Method A: Off-white solid (120 mg, 28%); ¹H NMR (500 MHz, DMSO-d₆) δ 8.55 (s, 1H), 8.15 (s, 1H), 8.08 (d, J=1.9 Hz, 1H), 7.59 (dt, J=8.3, 1.9 Hz, 1H), 7.56 (dd, J=8.2, 1.8 Hz, 1H), 3.99 (s, 3H), 3.21-3.11 (m, 2H), 2.91-2.77 (m, 2H), 1.87-1.68 (m, 4H); ¹³C NMR (126 MHz, DMSO) δ 169.32, 166.59, 153.43, 150.43, 140.96, 140.33, 138.84, 131.42, 129.03, 128.38, 125.04, 123.58, 122.59, 118.02, 116.40, 111.98, 53.45, 30.28, 27.43, 21.69, 21.65. HRMS (ESI) m/z calcd for C₂₃H₂₀N₅O₂S [M+H]⁺ 430.1338, found 430.1348.

### N-(6-(8,9,10,11-Ttetrahydro-3H-pyrazolo[4,3-a]phenanthridin-7-yl)benzo[d]thiazol-2-yl)acetamide

Compound HSD992 (93 mg, 0.25 mmol) was dissolved in a mixture of DMF (2 mL) and triethylamine (2 equiv), followed by addition of acetyl chloride (30 mg, 1.5 mmol). After that reaction was continued for overnight at room temperature. After completion of reaction, reaction mixture was extracted with ethyl acetate (2×20 mL) and washed with brine solution and the crude was purified with dichloromethane:methanol (90:10) by flash column chromatography to get the desired product as yellow solid (73 mg, 70%).

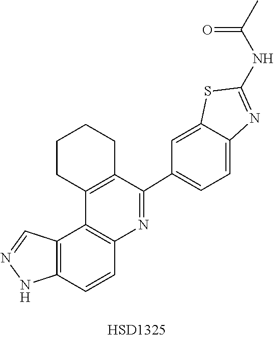 [SMILES: CC(=O)Nc1nc2ccc(-c3nc4ccc5[nH]ncc5c4c4c3CCCC4)cc2s1]

¹H NMR (500 MHz, Methanol-d₄) δ 8.76 (s, 1H), 8.38 Id, J=9.3 Hz, 1H), 8.31-8.24 (m, 1H), 8.20 (d, J=9.3 Hz, 1H), 7.88-7.84 (m, 1H), 7.76 (d, J=8.4 Hz, 1H), 4.32 (s, 3H), 3.67 (t, J=6.3 Hz, 2H), 2.95 (t, J=6.1 Hz, 2H), 2.26-2.18 (m, 2H), 1.99-1.87 (m, 2H); ¹³C NMR (126 MHz, MeOD) δ 170.98, 155.22, 149.49, 141.56, 138.36, 135.15, 134.18, 132.65, 129.29, 127.49, 125.42, 124.19, 123.58, 118.90, 118.87, 115.92, 114.54, 35.36, 31.10, 27.83, 21.22, 21.15; HRMS (ESI) m/z calcd for C₂₃H₂₀N₅O₂S [M+H]⁺ 414.1389, found 414.1388.

### 7-(3-(Trifluoromethyl)-1H-pyrazol-4-yl)-8,9,10,11-tetrahydro-3H-imidazo[4,5-a]phenanthridine

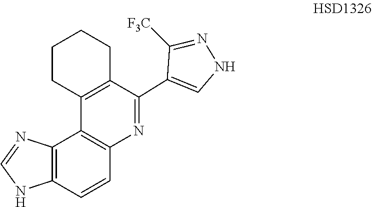 [SMILES: FC(F)(F)c1n[nH]cc1-c1nc2ccc3[nH]cnc3c2c2c1CCCC2]

Method A: Off-white solid (75 mg, 21%). ¹H NMR (500 MHz, Methanol-d₄) δ 8.30 (s, 1H), 7.99 (d, J=1.2 Hz, 1H), 7.93 (d, J=9.0 Hz, 1H), 7.83 (d, J=8.9 Hz, 1H), 3.68 (d, J=6.7 Hz, 2H), 2.68 (t, J=6.3 Hz, 2H), 2.08-1.98 (m, 2H), 1.91-1.83 (m, 2H); ¹³C NMR (126 MHz, MeOD) δ 149.00, 145.68, 143.27, 142.66, 140.11, 130.25, 130.05, 124.01, 122.80 (q, J=270.51 Hz), 118.99, 118.30, 115.91, 112.78, 94.71, 29.26, 27.75, 22.04, 21.86. HRMS (ESI) m/z calcd for C₁₈H₁₅F₃N₅ [M+H]⁺ 358.1280, found 358.1286.

### 2-(trifluoromethyl)-7-(3-(trifluoromethyl)-1H-pyrazol-4-yl)-8,9,10,11-tetrahydro-3H-imidazo[4,5-a]phenanthridine

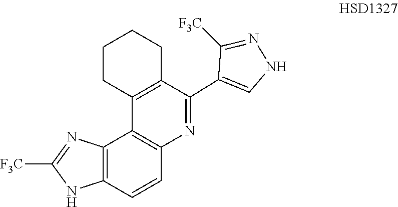 [SMILES: FC(F)(F)c1nc2c(ccc3nc(-c4c[nH]nc4C(F)(F)F)c4c(c32)CCCC4)[nH]1]

Method A: Off-white solid (106 mg, 25%). ¹H NMR (500 MHz, Methanol-d₄) δ 7.99 (s, 1H), 7.90 (d, J=9.1 Hz, 1H), 7.85 (d, J=9.1 Hz, 1H), 3.91-3.75 (m, 2H), 2.68 (t, J=6.3 Hz, 2H), 2.07-1.92 (m, 2H), 1.94-1.77 (m, 2H), ¹³C NMR (126 MHz, MeOD) δ 149.46, 144.26, 143.57, 140.04, 139.76, 137.30, 131.82, 130.86, 130.17, 126.46, 124.94 (q, J=269.64 Hz), 120.67, 120.32 (q, J=268.64 Hz), 118.93, 114.85, 29.42, 27.82, 21.99, 21.94. HRMS (ESI) m/z calcd for C₁₉H₁₄F₆N₅ [M+H]⁺ 426.1153, found 426.1155.

### Methyl 7-(3-(trifluoromethyl)-1H-pyrazol-4-yl)-8,9,10,11-tetrahydro-3H-pyrazolo[4,3-a]phenanthridine-1-carboxylate

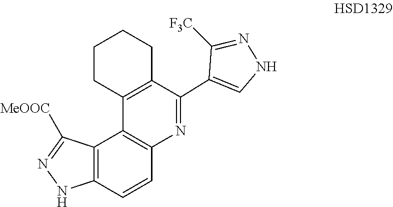 [SMILES: COC(=O)c1n[nH]c2ccc3nc(-c4c[nH]nc4C(F)(F)F)c4c(c3c12)CCCC4]

Method A: Off-white solid (83 mg, 20%). ¹H NMR (500 MHz, Methanol-d₄) δ 8.36-8.34 (m, 1H), 8.33 (d, J=9.2 Hz, 1H), 8.07 (d, J=9.2 Hz, 1H), 4.09 (s, 3H), 3.41 (t, J=6.0 Hz, 2H), 2.83 (t, J=6.5 Hz, 2H), 2.04-1.96 (m, 2H), 1.94-1.87 (m, 2H); ¹³C NMR (126 MHz, MeOD) δ 165.69, 155.80, 143.04, 140.76, 140.09, 139.80, 136.05, 134.16, 132.40, 124.22, 122.30 (268.38), 120.37, 111.65, 115.75, 109.90, 52.41, 31.10, 26.62, 21.03, 20.75; HRMS (ESI) m/z calcd for C₂₀H₁₇F₃N₅O₂ [M+H]⁺ 416.1334, found 416.1343.

### 7-(3-(Trifluoromethyl)-1H-pyrazol-4-yl)-8,9,10,11-tetrahydro-3H-pyrazolo[4,3-a]phenanthridine-9-carbonitrile

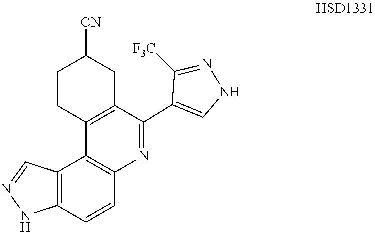 [SMILES: N#CC1CCc2c(c(-c3c[nH]nc3C(F)(F)F)nc3ccc4[nH]ncc4c23)C1]

Method A: Off-white solid (100 mg, 26%). ¹H NMR (500 MHz, DMSO-d₆) δ δ 8.78 (s, 1H), 8.39 (s, 1H), 8.13 (d, J=9.5, 4.5 Hz, 1H), 7.97 (d, J=9.2, 4.4 Hz, 1H), 3.61-3.50 (m, 2H), 3.48-3.36 (m, 1H), 3.12-2.95 (m, 2H), 2.37-2.22 (m, 2H). HRMS (ESI) m/z calcd for C₁₉H₁₄F₃N₆ [M+H]⁺ 383.1232, found 383.1237.

### 1-Methyl-7-(3-(trifluoromethyl)-1H-pyrazol-4-yl)-8,9,10,11-tetrahydro-3H-pyrazolo[4,3-a]phenanthridine-9-carbonitrile

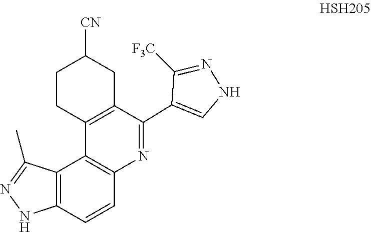 [SMILES: Cc1n[nH]c2ccc3nc(-c4c[nH]nc4C(F)(F)F)c4c(c3c12)CCC(C#N)C4]

Method A: Off-white solid (59 mg, 15%). ¹H NMR (500 MHz, Methanol-d₄) δ 8.37 (d, J=1.0 Hz, 1H), 8.21 (d, J=9.2 Hz, 1H), 7.89 (d, J=9.2 Hz, 1H), 3.94 (t, J=6.6 Hz, 2H), 3.49-3.42 (m, 1H), 3.20 (dd, J=17.3, 5.9 Hz, 1H), 3.09 (d, J=7.5 Hz, 1H), 3.06 (s, 3H), 2.39 (m, J=12.6, 6.0, 3.4 Hz, 1H), 2.21 (m, J=13.3, 8.5, 6.5 Hz, 1H); ¹³C NMR (126 MHz, Methanol-d₄) δ 151.72, 141.79, 137.08, 132.47, 129.46, 126.34, 123.81, 123.71, 123.64, 122.20 (q, J=270), 112.62, 109.78, 109.78, 30.62, 30.56, 29.87, 24.22, 23.60, 23.54, 17.53.

### 8,9,10,11-Tetrahydro-3H-pyrazolo[4,3-a]phenanthridine-7-carboxylic acid (HSD1251)

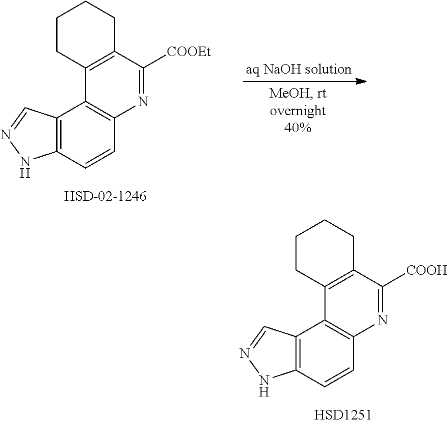 [SMILES: CCOC(=O)c1nc2ccc3[nH]ncc3c2c2c1CCCC2 ; O=C(O)c1nc2ccc3[nH]ncc3c2c2c1CCCC2]

Off-white solid (40%). ¹H NMR (500 MHz, DMSO-d₆) δ 8.60 (s, 1H), 7.90 (d, J=9.0 Hz, 1H), 7.83 (d, J=9.5 Hz, 1H), 3.32-3.26 (m, 1H), 3.06-2.96 (m, 2H), 2.03-1.92 (m, 2H), 1.87-1.75 (m, 2H).

### N-Cyclopropyl-8,9,10,11-tetrahydro-3H-pyrazolo[4,3-a]phenanthridine-7-carboxamide

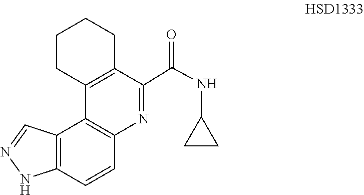 [SMILES: O=C(NC1CC1)c1nc2ccc3[nH]ncc3c2c2c1CCCC2]

To a solution of HSD1251 (50 mg, 0.18 mmol) in DMF (2.5 mL) was added cyclopropylamine (0.19 mmol), HOBt (0.18 mmol), EDCl.HCl (0.18 mmol) and DIPEA (0.39 mmol). Reaction was continued for 12 h at room temperature and monitored by TLC. After completion, reaction mixture was extracted with ethyl acetate and washed with brine solution and dried over Na₂SO₄. Crude was purified with flash chromatography to get the desired product as white solid (70% yield, 39 mg). NMR (500 MHz, DMSO-d₆) δ 8.59-8.53 (m, 2H), 7.91-7.82 (m, 2H), 3.05-2.97 (m, 2H), 2.92-2.82 (m, 1H), 2.02-1.92 (m, 2H), 1.85-1.74 (m, 2H), 0.77-0.66 (m, 2H), 0.62-0.54 (m, 2H); ¹³C NMR (126 MHz, DMSO) δ 169.02, 150.75, 143.04, 142.46, 138.96, 136.49, 129.42, 129.13, 123.13, 116.31, 114.78, 29.62, 26.70, 23.08, 22.44, 22.07, 6.21. HRMS (ESI) m/z calcd for C₁₈H₁₉N₄O [M+H]⁺ 307.1559, found 307.1565.

### 9-Methyl-7-(3-(trifluoromethyl)-1H-pyrazol-4-yl)-8,9,10,11-tetrahydro-3H-indazolo[5,4-c][2,71]naphthyridine

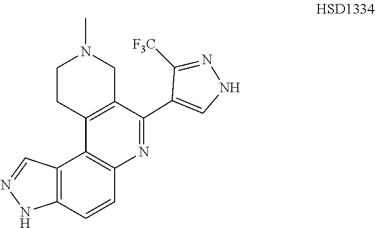 [SMILES: CN1CCc2c(c(-c3c[nH]nc3C(F)(F)F)nc3ccc4[nH]ncc4c23)C1]

Method A: Off-white solid (96 mg, 26%). ¹H NMR (500 MHz, Methanol-d₄) δ 8.19 (s, 1H), 8.05-7.98 (m, 1H), 7.26 (d, J=8.9 Hz, 1H), 6.81 (d, J=8.9 Hz, 1H), 6.49-6.40 (m, 1H), 4.26 (d, J=11.1 Hz, 1H), 3.66-3.51 (m, 1H), 3.06-2.89 (m, 2H), 2.65-2.53 (m, 1H), 2.32 (s, 3H), 1.96 (t, J=10.9 Hz, 1H); ¹³C NMR (126 MHz, MeOD) δ 139.53, 136.09, 131.98, 131.66, 129.91, 123.14 (q, J=269.6 Hz), 119.98, 118.88, 117.25, 110.66, 107.76, 55.06, 54.96, 51.22, 44.41, 40.51.

### 6-(9-Methyl-8,9,10,11-tetrahydro-3H-indazolo[5,4-c][2,7]naphthyridin-7-yl)benzo[d]thiazol-2-amine

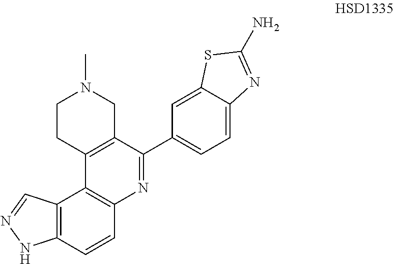 [SMILES: CN1CCc2c(c(-c3ccc4nc(N)sc4c3)nc3ccc4[nH]ncc4c23)C1]

Method A: Off-white solid (81 mg, 21%). ¹H NMR (500 MHz, DMSO-d₆) δ 7.74 (d, J=1.7 Hz, 1H), 7.45 (s, 2H), 7.32 (d, J=8.1 Hz, 1H), 7.26 (dd, J=8.2, 1.8 Hz, 1H), 7.19 (d, J=8.8 Hz, 1H), 6.79 (d, J=8.9 Hz, 1H), 6.33 (d, J=4.8 Hz, 1H), 5.95 (s, 1H), 3.94 (d, J=11.0 Hz, 1H), 3.46-3.40 (m, 2H), 2.80-2.71 (m, 2H), 2.25-2.17 (m, 1H), 2.12 (s, 3H), 1.77 (t, J=10.6 Hz, 1H); ¹³C NMR (126 MHz, DMSO) δ 167.11, 153.11, 140.29, 135.76, 134.70, 132.26, 132.06, 131.56, 125.86, 120.50, 119.90, 118.66, 118.03, 117.84, 111.01, 107.40, 61.27, 55.89, 46.15.

### N-(7-(3-(Trifluoromethyl)-1H-pyrazol-4-yl)-8,9,10,11-tetrahydro-3H-pyrazolo[4,3-a]phenanthridin-9-yl)acetamide

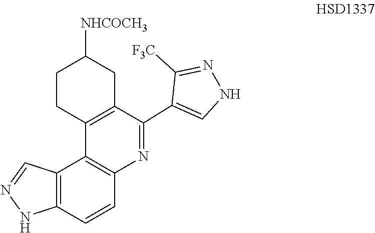

Method A: Off-white solid (137 mg, 33%). ¹H NMR (500 MHz, Methanol-d₄) δ 8.56 (s, 1H), 8.02 (s, 1H), 7.86 (s, 2H), 4.25-4.09 (m, 1H), 3.63-3.55 (m, 1H), 3.50-3.42 (m, 2H), 3.02-2.92 (m, 1H), 2.71-2.59 (m, 1H), 2.40-2.30 (m, 1H), 1.98-1.90 (m, 4H); ¹³C NMR (126 MHz, DMSO) δ 169.42, 153.60, 148.04, 140.41, 138.26, 134.78, 129.37, 127.68, 122.95, 122.23, 115.48, 110.76, 44.12, 34.25, 29.21, 27.37, 23.11. HRMS (ESI) m/z calcd for C₂₀H₁₈F₃N₆O [M+H]⁺ 415.1494, found 415.1498.

### 7-(3-(Difluoromethyl)-1-methyl-1H-pyrazol-4-yl)-5-fluoro-8,9,10,11-tetrahydro-3H-pyrazolo[4,3-a]phenanthridine

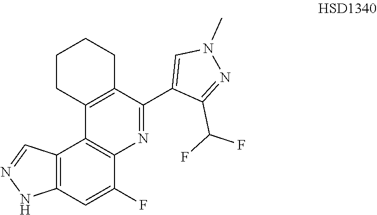 [SMILES: Cn1cc(-c2nc3c(F)cc4[nH]ncc4c3c3c2CCCC3)c(C(F)F)n1]

Method A: Off-white solid (37 mg, 10%). ¹H NMR (500 MHz, DMSO-d₆) δ 8.50 (s, 1H), 8.27 (s, 1H), 7.69-7.62 (m, 1H), 7.49 (t, J=54.7 Hz, 1H), 3.97 (s, 3H), 3.32-3.24 (m, 2H), 2.94-2.83 (m, 2H), 2.02-1.92 (m, 2H), 1.85-1.73 (m, 2H); ¹³C NMR (126 MHz, DMSO) δ 158.06, 156.05, 148.40, 144.52, 144.34, 144.16, 143.02, 136.82, 136.43, 134.20, 132.97, 131.01, 122.11, 119.91, 113.31, 112.93, 111.45, 109.58, 98.48, 98.29, 31.15, 29.80, 28.54, 22.27.

### 7-(3-(Trifluoromethyl)-1H-pyrazol-4-yl)-8,9,10,11-tetrahydro-3H-indazolo[5,4-c][2,7]naphthyridine

In this case tert-butyl 4-oxopiperidine-1-carboxylate was used as cyclic ketone using method A. Boc-group was deprotected in-situ due to presence of HCl in the reaction and desired compound was precipitated out in the reaction mixture which was filtered and washed with ethanol.

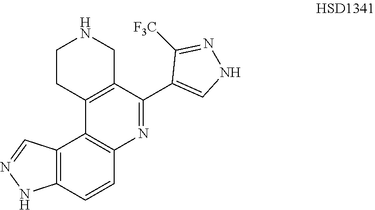 [SMILES: FC(F)(F)c1n[nH]cc1-c1nc2ccc3[nH]ncc3c2c2c1CNCC2]

Method A: Off-white solid (100 mg, 28%). ¹H NMR (500 MHz, DMSO-d₆) δ 9.70 (s, 2H), 8.60 (s, 1H), 8.34 (s, 1H), 7.97 (dd, J=9.2, 3.7 Hz, 1H), 7.84 (d, J=9.1 Hz 1H), 4.37-4.31 (m, 2H), 3.70-3.62 (m, 2H), 3.61-3.53 (m, 2H); ¹³C NMR (126 MHz, DMSO) δ 146.11, 144.36, 139.64 (q, J 36.54 Hz), 138.90, 138.14, 135.96, 131.61, 129.36, 123.35 (q, J=270.9 Hz), 122.70, 120.58, 117.94, 115.98, 43.23, 25.91; C₁₇H₁₄F₃N₆ [M+H]⁺ 359.1232, found 359.1233.

### 5-fluoro-7-(3-(trifluoromethyl)-1H-pyrazol-4-yl)-8,9,10,11-tetrahydro-3H-indazolo[5,4-c][2,7]naphthyridine

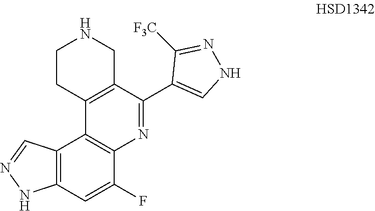 [SMILES: Fc1cc2[nH]ncc2c2c3c(c(-c4c[nH]nc4C(F)(F)F)nc12)CNCC3]

Method A: Same cyclic ketone as in HSD1341: Off-white solid (60 mg, 16%). ¹H NMR (500 MHz, DMSO-d₆) δ 8.60 (s, 1H), 8.28 (s, 1H), 7.71 (d, J=10.0 Hz, 1H), 3.85 (s, 2H), 3.24 (q, J=5.1, 4.2 Hz, 2H), 3.16 (t, J=4.4 Hz, 2H); ¹³C NMR (126 MHz, DMSO) δ 158.12 (d, J=253.26 Hz), 146.55, 141.41, 139.52, 139.23, 138.48, 134.96, 131.48, 123.42 (q, J=269.64 Hz), 122.86, 118.58, 112.68, 99.76, 97.55, 55.01, 47.19, 42.89; HRMS (ESI) m/z calcd for C₁₇H₁₃F₄N₆[M+H]⁺ 377.1138, found 377.1138.

### N-(4-((4-methylpiperazin-1-yl)methyl)-3-(trifluoromethyl)phenyl)-8,9,10,11-tetrahydro-3H-pyrazolo[4,3-a]phenanthridine-7-carboxamide

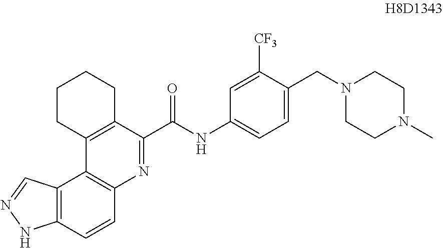 [SMILES: CN1CCN(Cc2ccc(NC(=O)c3nc4ccc5[nH]ncc5c4c4c3CCCC4)cc2C(F)(F)F)CC1]

To a solution of HSD1251 (50 mg, 0.18 mmol) in DMF (2.5 mL) was added 4-((4-methylpiperazin-1-yl)methyl)-3-(trifluoromethyl)aniline (0.19 mmol). HATU (0.18 mmol), and DIPEA (0.39 mmol). Reaction was continued for 12 h at room temperature and monitored by TLC. After completion, reaction mixture was extracted with ethyl acetate and washed with brine solution and dried over Na₂SO₄. Crude was purified with flash chromatography to get the desired product as Off-white solid (50% yield, 47 mg). ¹H NMR (500 MHz, DMSO-d₆) δ 10.93 (s, 1H), 8.63 (s, 1H), 8.29 (d, J=2.2 Hz, 1H), 8.05 (dd, J=8.5, 2.3 Hz, 1H), 7.98-7.89 (m, 2H), 7.71 (d, J=8.5 Hz, 1H), 3.56 (s, 2H), 3.34-3.30 (m, 2H), 3.13 (t, J=6.2 Hz, 2H), 2.37 (s, 8H), 2.14 (s, 3H), 1.99 (ddt, J=8.9, 6.3, 2.9 Hz, 2H), 1.87-1.79 (m, 2H); ¹³C NMR (126 MHz, DMSO) δ 166.54, 149.19, 143.71, 142.60, 138.52, 132.53, 131.81, 129.74, 129.42, 128.11 (q, J=28.9 Hz), 125.91 (q, J=274.6 Hz), 123.78, 123.53, 117.19, 117.14, 116.03, 57.94, 55.22, 53.19, 46.22, 29.73, 26.89, 22.37, 22.07; HRMS (ESI) m/z calcd for C₂₈H₃₀F₃N₆O [M+H]⁺ 523.2433, found 523.2433.

### 7-(2-aminobenzo[d]thiazol-6-yl)-3H-pyrazolo[4,3-f]quinoline-9-carboxylic acid

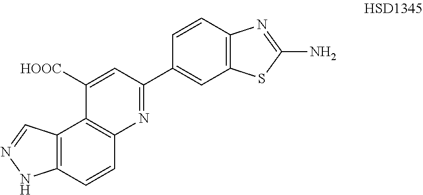 [SMILES: Nc1nc2ccc(-c3cc(C(=O)O)c4c(ccc5[nH]ncc54)n3)cc2s1]

Method A: Brown solid (126 mg, 35%). ¹H NMR (500 MHz, DMSO-d₆) δ 8.70 (d, J=1.8 Hz, 1H), 8.48-8.35 (m, 3H), 8.32 (s, 1H), 8.25 (dd, J=8.5, 1.9 Hz, 1H), 8.00 (d, J=9.2 Hz, 1H), 7.96 (d, J=9.1 Hz, 1H), 7.51 (d, J=8.5 Hz, 1H); ¹³C NMR (126 MHz, DMSO) δ 170.36, 169.08, 153.15, 146.62, 138.70, 132.42, 129.93, 129.24, 125.78, 120.77, 117.16, 117.04, 115.21.

### 7-(Isoquinolin-6-yl)-8,9,10,11-tetrahydro-3H-pyrazolo[4,3-a]phenanthridine

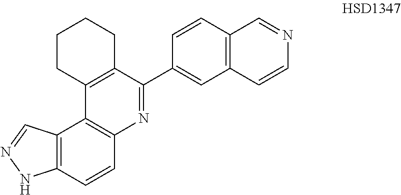 [SMILES: c1cc2cc(-c3nc4ccc5[nH]ncc5c4c4c3CCCC4)ccc2cn1]

Method A: Off-white solid (178 mg, 51%). ¹H NMR (500 MHz, DMSO-d₆) δ 9.34 (s, 1H), 8.65-8.45 (m, 2H), 8.23-8.08 (m, 2H), 7.94-7.75 (m, 4H), 3.36-3.01 (m, 2H), 2.84-2.64 (m, 2H), 2.03-1.80 (m, 2H), 1.74-1.50 (m, 2H); ¹³C NMR (126 MHz, DMSO) δ 155.87, 152.56, 143.58, 143.17, 142.64, 138.79, 136.42, 135.77, 135.39, 129.57, 129.30, 127.89, 127.63, 127.02, 124.06, 122.13, 121.09, 116.40, 114.60, 29.61, 28.83, 22.51, 22.40.

### 7-(Isoquinolin-8-yl)-8,9,10,11-tetrahydro-3H-pyrazolo[4,3-a]phenanthridine

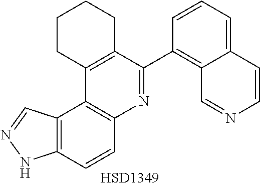 [SMILES: c1cc(-c2nc3ccc4[nH]ncc4c3c3c2CCCC3)c2cnccc2c1]

Method A: Brown solid (140 mg, 40%). ¹H NMR (500 MHz, Methanol-d₄) δ 8.67 (d, J=1.0 Hz, 1H), 8.61 (s, 1H), 8.46 (d, J=5.8 Hz, 1H), 8.07 (dt, J=8.4, 1.1 Hz, 1H), 7.95-7.90 (m, 2H), 7.86 (s, 2H), 7.70 (dd, J=7.0, 1.1 Hz, 1H), 3.45-3.36 (m, 2H), 2.71-2.57 (m, 1H), 2.42-2.30 (m, 1H), 2.08-2.02 (m, 2H), 1.84-1.71 (m, 2H); ¹³C NMR (126 MHz, MeOD) δ 154.18, 149.50, 144.02, 143.31, 141.71, 138.68, 136.43, 130.58, 130.53, 128.48, 128.34, 126.97, 126.93, 122.66, 121.40, 115.96, 29.52, 27.69, 22.02, 21.75.

### 7-(Isoquinolin-7-yl)-8,9,10,11-tetrahydro-3H-pyrazolo[4,3-a]phenanthridine

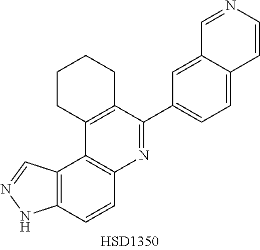 [SMILES: c1cc2ccc(-c3nc4ccc5[nH]ncc5c4c4c3CCCC4)cc2cn1]

Method A: Off-white solid (105 mg, 30%). ¹H NMR (500 MHz, Methanol-d₄) δ 9.33 (d, J=1.4 Hz, 1H), 8.61 (s, 1H), 8.50 (dd, J=5.9, 1.4 Hz, 1H), 8.29 (s, 1H), 8.09 (d, J=8.4 Hz, 1H), 7.98 (dt, J=8.4, 1.6 Hz, 1H), 7.93-7.85 (m, 2H), 3.47-3.37 (m, 2H), 2.84 (t, J=6.2 Hz, 2H), 2.17-2.08 (m, 2H), 1.89-1.82 (m, 2H); ¹³C NMR (126 MHz, MeOD) δ 156.08, 152.22, 143.84, 143.20, 141.99, 140.04, 138.79, 136.00, 135.65, 132.00, 129.69, 128.57, 128.47, 127.97, 126.35, 122.41, 120.88, 113.80, 29.64, 28.49, 22.13, 22.04.

### 7-(Quinolin-2-yl)-8,9,10,11-tetrahydro-3H-pyrazolo[4,3-a]phenanthridine

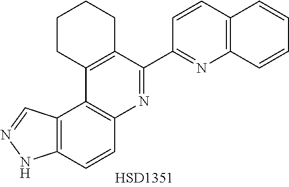 [SMILES: c1ccc2nc(-c3nc4ccc5[nH]ncc5c4c4c3CCCC4)ccc2c1]

Method A: Off-white solid (150 mg, 43%). ¹H NMR (500 MHz, DMSO-d₆) δ 8.61 (s, 1H), 8.50 (d, J=8.5 Hz, 1H), 8.09-8.03 (m, 2H), 8.00 (d, J 8.4 Hz, 1H), 7.89 (s, 2H), 7.80 (dd, J=8.3, 6.8, 1.5 Hz, 1H), 7.69-7.62 (m, 1H), 3.38 (d, J=6.6 Hz, 2H), 3.09 (t, J=6.2 Hz, 2H), 2.02 (dp, J=9.3, 3.2 Hz, 2H), 1.82-1.71 (m, 2H); ¹³C NMR (126 MHz, DMSO) δ 159.31, 154.53, 146.93, 143.32, 143.03, 138.94, 136.97, 136.58, 130.38, 130.26, 129.68, 129.51, 128.37, 127.38, 122.95, 122.49, 116.48, 114.65, 29.86, 28.31, 22.54, 22.43; HRMS (ESI) m/z calcd for C₂₃H₁₉N₄ [M+H]⁺ 351.1610, found 351.1608.

### 7-(isoquinolin-4-yl)-8,9,10,11-tetrahydro-3H-pyrazolo[4,3-a]phenanthridine

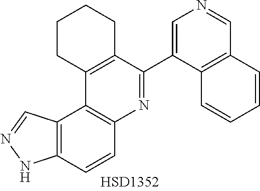 [SMILES: c1ccc2c(-c3nc4ccc5[nH]ncc5c4c4c3CCCC4)cncc2c1]

Method A: Off-white solid (262 mg, 75%). ¹H NMR (500 MHz, DMSO-d₆) δ 9.41 (d, J=0.9 Hz, 1H), 8.61 (s, 1H), 8.50 (s, 1H), 8.28-8.14 (m, 1H), 7.94-7.80 (m, 2H), 7.73-7.62 (m, 2H), 7.38-7.31 (m, 1H), 3.44-3.35 (m, 2H), 2.64-2.54 (m, 1H), 2.40-2.27 (m, 1H), 2.00-1.95 (m, 3H), 1.77-1.65 (m, 2H); ¹³C NMR (126 MHz, DMSO) δ 153.43, 152.80, 143.82, 142.90, 142.62, 138.81, 136.47, 134.12, 132.07, 131.60, 130.70, 129.60, 128.54, 128.33, 128.02, 124.62, 122.35, 116.52, 114.67, 29.59, 27.87, 22.50, 22.16. HRMS (ESI) m/z calcd for C₂₃H₁₉N₄ [M+H]+ 351.1610. found 351.1611.

### 9-(Pyridin-2-yl)-7-(3-(trifluoromethyl)-1H-pyrazol-4-yl)-3H-pyrazolo[4,3-f]quinoline

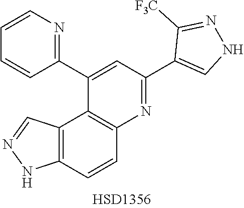 [SMILES: FC(F)(F)c1n[nH]cc1-c1cc(-c2ccccn2)c2c(ccc3[nH]ncc32)n1]

Method C: Off-white solid (160 mg, 42%). ¹H NMR (500 MHz, Methanol-d₄) δ 8.80 (ddt, J=5.0, 1.7, 0.7 Hz, 1H), 8.37 (s, 1H), 8.14 (td, J=7.7, 1.7 Hz, 1H), 8.01 (d, J=9.2 Hz, 1H), 7.92 (dd, J=9.3, 0.9 Hz, 1H), 7.82-7.65 (m, 3H), 6.69 (s, 1H); ¹³C NMR (126 MHz, MeOD) δ 157.76, 157.73, 149.27, 147.72, 146.52, 145.30, 139.22 (q, J=36.54 Hz), 138.53, 131.12, 129.05, 129.02, 124.48, 124.45, 124.33, 123.02 (q, J=268.38 Hz), 121.14, 120.71, 119.57, 115.74.

### 7-(Quinolin-6-yl)-8,9,10,11-tetrahydro-3H-pyrazolo[4,3-a]phenanthridine

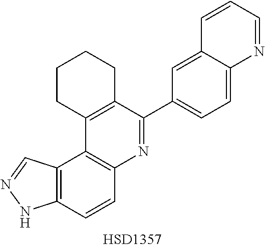 [SMILES: c1cnc2ccc(-c3nc4ccc5[nH]ncc5c4c4c3CCCC4)cc2c1]

Method A: Off-white solid (192 mg, 55%). ¹H NMR (500 MHz, DMSO-d₆) δ 9.01 (dd, J=4.6, 1.7 Hz, 1H), 8.62 (s, 1H), 8.13 (d, J=8.5 Hz, 1H), 7.90 (d, J=9.1 Hz, 1H), 7.82 (d, J=8.8 Hz, 1H), 7.77 (t, J=7.6 Hz, 1H), 7.59-7.52 (m, 1H), 7.49 (t, J=7.6 Hz, 1H), 7.34 (d, J=8.3 Hz, 1H), 3.49-3.36 (m, 2H), 2.61-2.54 (m, 2H), 2.39-2.23 (m, 1H), 2.02-1.87 (m, 2H), 1.77-1.60 (m, 2H); ¹³C NMR (126 MHz, DMSO) δ 153.87, 150.73, 148.39, 146.91, 143.65, 142.78, 138.83, 136.48, 130.02, 129.95, 129.78, 129.52, 127.55, 126.67, 125.91, 122.43, 121.73, 116.49, 114.82, 29.57, 27.63, 22.47, 22.06 HRMS (ESI) m/z calcd for C₂₃H₁₉N₄ [M+H]⁺ 351.1610. found 351.1611.

### 7-(Quinolin-3-yl)-8,9,10,11-tetrahydro-3H-pyrazolo[4,3-a]phenanthridine

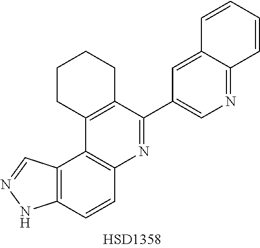 [SMILES: c1ccc2ncc(-c3nc4ccc5[nH]ncc5c4c4c3CCCC4)cc2c1]

Method A: Off-white solid (162 mg, 46%). ¹H NMR (500 MHz, DMSO-d₆) δ 9.14 (s, 1H), 8.61-8.60 (m, 2H), 8.10 (t, J=7.25 Hz, 2H), 7.89 (s, 2H), 7.83 (t, J=6.95 Hz, 1H), 7.68 (d, J=7.8 Hz, 1H), 3.39 (t, J=5.65 Hz, 2H), 2.92 (t, J=5.75 Hz, 2H), 2.06-2.02 (m, 2H), 2.07-1.98 (m, 2H), 1.78-1.77 (m, 2H); ¹³C NMR (126 MHz, MeOD) δ 153.88, 151.60, 147.28, 143.90, 142.87, 138.80, 136.53, 136.17, 134.00, 130.35, 129.97, 129.70, 129.19, 128.96, 127.45, 122.16, 116.44, 114.74, 29.75, 28.86, 22.58; HRMS (ESI) m/z calcd for C₂₃H₁₉N₄ [M+H]⁺ 351.1610, found 351.1613.

### 3-Chloro-4-(8,9,10,11-tetrahydro-3H-pyrazolo[4,3-a]phenanthridin-7-yl)phenol

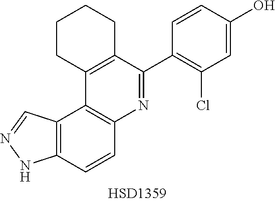 [SMILES: Oc1ccc(-c2nc3ccc4[nH]ncc4c3c3c2CCCC3)c(Cl)c1]

Method A: Off-white solid (178 mg, 51%). ¹H NMR (500 MHz, Methanol-d₄) δ 8.55 (s, 1H), 7.88-7.76 (m, 2H), 7.18 (d, J=8.3 Hz, 1H), 6.97 (d, J=2.4 Hz, 1H), 6.88 (dd, J=8.3, 2.4 Hz, 1H), 3.39-3.31 (m, 2H), 2.67 (dt, J=17.3, 6.3 Hz, 1H), 2.54 (dt, J=17.2, 6.2 Hz, 1H), 2.07-1.98 (m, 2H), 1.91-1.74 (m, 2H); ¹³C NMR (126 MHz, MeOD) δ 158.50, 155.40, 143.33, 142.83, 138.70, 135.81, 132.99, 130.85, 130.10, 128.23, 122.36, 116.12, 115.69, 114.11, 113.67, 29.44, 27.16, 22.10, 21.82. HRMS (ESI) m/z calcd for C₂₀H₁₇ClN₃O [M+H]⁺ 350.1060, found 350.1063.

### 3-Fluoro-5-(8,9,10,11-tetrahydro-3H-pyrazolo[4,3-a]phenanthridin-7-yl)phenol

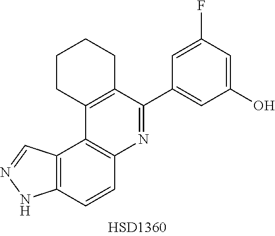 [SMILES: Oc1cc(F)cc(-c2nc3ccc4[nH]ncc4c3c3c2CCCC3)c1]

Method A: Off-white solid (117 mg, 35%). ¹H NMR (500 MHz, Methanol-d₄) δ 8.59 (s, 1H), 7.91-7.82 (m, 2H), 7.20 (d, J=8.3 Hz, 1H), 6.98 (d, J=2.4 Hz, 1H), 6.89 (dd, J=8.3, 2.4 Hz, 1H), 3.48-3.35 (m, 2H), 2.70 (dt, J=17.3, 6.3 Hz, 1H), 2.57 (dt, J=17.2, 6.2 Hz, 1H), 2.10-2.03 (m, 2H), 1.92-1.82 (m, 2H); ¹³C NMR (126 MHz, MeOD) δ 158.52, 155.48, 143.37, 142.83, 135.85, 132.99, 130.91, 130.84, 130.11, 128.26, 122.39, 116.16, 115.69, 114.11, 113.71, 29.48, 27.18, 22.13, 21.84.

### 7-(Isoquinolin-1-yl)-8,9,10,11-tetrahydro-3H-pyrazolo[4,3-a]phenanthridine

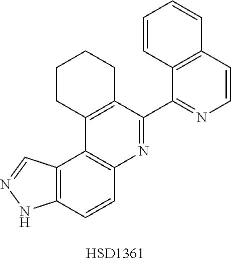 [SMILES: c1ccc2c(-c3nc4ccc5[nH]ncc5c4c4c3CCCC4)nccc2c1]

Method A: Off-white solid (172 mg, 49%). ¹H NMR (500 MHz, Methanol-d₄) δ 8.67 (s, 1H), 8.59 (dd, J=5.9, 1.3 Hz, 1H), 8.07 (d, J=8.3 Hz, 1H), 7.98 (d, J=5.8 Hz, 1H), 7.90 (s, 2H), 7.81 (ddd, J=8.3, 6.7, 1.4 Hz, 1H), 7.62-7.55 (m, 1H), 7.52 (d, J=8.4 Hz, 1H), 3.49 (d, J=8.1 Hz, 2H), 2.54 (s, 2H), 2.13-2.05 (m, 2H), 1.87-1.76 (m, 2H); ¹³C NMR (126 MHz, MeOD) δ 158.92, 153.77, 144.17, 140.73, 136.98, 131.02, 130.39, 128.41, 128.05, 127.21, 126.99, 126.27, 123.01, 121.47, 29.53, 26.71, 22.03, 21.60. HRMS (ESI) m/z calcd for C₂₃H₁₉N₄ [M+H]⁺ 351.1610, found 351.1606.

### 1-Methoxy-7-(3-(trifluoromethyl)-1H-pyrazol-4-yl)-8,9,10,11-tetrahydro-3H-pyrazolo[4,3-a]phenanthridine

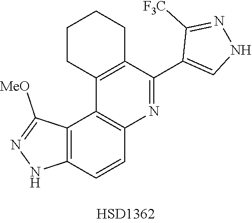 [SMILES: COc1n[nH]c2ccc3nc(-c4c[nH]nc4C(F)(F)F)c4c(c3c12)CCCC4]

Method A: Off-white solid (163 mg, 42%). ¹H NMR (500 MHz, Methanol-d₄) δ 8.22 (s, 1H), 7.92 (d, J=9.2 Hz, 1H), 7.86 (d, J=9.2 Hz, 1H), 4.15 (d, J=1.1 Hz, 3H), 3.79 (t, J=6.1 Hz, 2H), 2.73 (t, J=6.2 Hz, 2H), 2.01-1.83 (m, 4H); ¹³C NMR (126 MHz, MeOD) δ 158.09, 154.86, 141.34, 135.93, 132.97, 131.61, 125.26, 123.06, 118.25, 118.16, 101.38, 56.07, 31.36, 27.62, 21.62, 21.18. HRMS (ESI) m/z calcd for C₁₉H₁₇F₃N₅O [M+H]⁺ 388.1385, found 388.1390.

### 1-Methyl-7-(1-methyl-3-(trifluoromethyl)-1H-pyrazol-4-yl)-8,9,10,11-tetrahydro-3H-pyrazolo[4,3-a]phenanthridine-9-carbonitrile

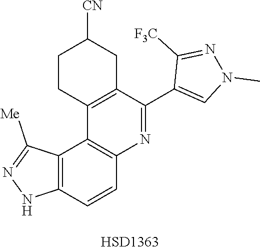 [SMILES: Cc1n[nH]c2ccc3nc(-c4cn(C)nc4C(F)(F)F)c4c(c3c12)CCC(C#N)C4]

Method A: Off-white solid (54 mg, 13%). ¹H NMR (500 MHz, Methanol-d₄) δ 7.96 (s, 1H), 7.85-7.75 (m, 2H), 4.07 (s, 3H), 3.74-3.64 (m, 2H), 3.14-3.07 (m, 1H), 3.03-2.96 (m, 1H), 2.33-2.25 (m, 1H), 2.13-2.04 (m, 1H); ¹³C NMR (126 MHz, MeOD) δ 147.28, 143.95, 143.52, 141.31, 141.20, 139.12, 132.56, 128.83, 126.48, 123.95, 122.46 (q, J=269.64 Hz), 121.58, 118.93, 114.92, 114.10, 38.46, 30.48, 29.49, 25.02, 24.24. HRMS (ESI) m/z calcd for C₂₁H₁₈F₃N₆ [M+H]⁺ 411.1545 found 411.1548.

### 7-(1-Methyl-3-(trifluoromethyl)-1H-pyrazol-4-yl)-8,9,10,11-tetrahydro-3H-pyrazolo[4,3-a]phenanthridine

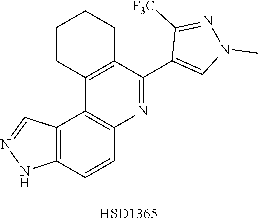 [SMILES: Cn1cc(-c2nc3ccc4[nH]ncc4c3c3c2CCCC3)c(C(F)(F)F)n1]

Method A: Off-white solid (112 mg, 30%). ¹H NMR (500 MHz, DMSO-d₆) δ 8.58-8.53 (m, 1H), 8.20-8.13 (m, 1H), 7.88-7.80 (m, 1H), 7.80-7.73 (m, 1H), 4.03-3.96 (m, 3H), 3.28-3.14 (m, 2H), 2.69 (t, J=6.6 Hz, 2H), 2.01-1.93 (m, 2H), 1.81-1.75 (m, 2H); ¹³C NMR (126 MHz, DMSO) δ 148.11, 143.48, 142.15, 138.76, 138.47, 136.40, 133.48, 130.33, 129.58, 123.20 (q, J=270.9 Hz), 121.98, 120.45, 116.40, 114.51, 29.54, 28.23, 22.57, 22.30. HRMS (ESI) m/z calcd for C₁₉H₁₇F₃N₅[M+H]⁺ 372.1436, found 372.1444.

### 7-(3-(Trifluoromethyl)-1H-pyrazol-4-yl)-3,8,10,11-tetrahydropyrazolo[4,3-f]thiopyrano[3,4-c]quinolone

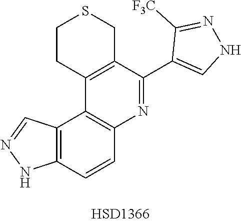 [SMILES: FC(F)(F)c1n[nH]cc1-c1nc2ccc3[nH]ncc3c2c2c1CSCC2]

Method A: Gray solid (124 mg, 33%). ¹H NMR (500 MHz, DMSO-d₆) δ 8.78 (s, 1H), 8.44 (s, 1H), 8.15 (d, J=9.1 Hz, 1H), 8.02 (d, J=9.2 Hz, 1H), 3.80 (s, 2H), 3.69 (t, J=6.1 Hz, 2H), 3.17 (t, J=6.0 Hz, 2H). HRMS (ESI) m/z calcd for C₁₇H₁₃F₃N₅S [M+H]⁺ 376.0844, found 376.0840.

### 1-(7-(3-(Trifluoromethyl)-1H-pyrazol-4-yl)-3,8,10,11-tetrahydro-9H-indazolo[5,4-c][2,7]naphthyridin-9-yl)ethan-1-one

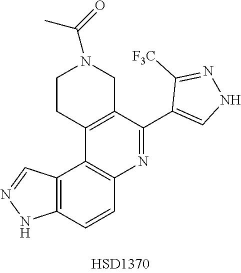 [SMILES: CC(=O)N1CCc2c(c(-c3c[nH]nc3C(F)(F)F)nc3ccc4[nH]ncc4c23)C1]

Method C: Off-white solid (60 mg, 15%). ¹H NMR (500 MHz, DMSO-d₆) δ 8.76 (d, J=27.3 Hz, 1H), 8.45 (d, J=10.1 Hz, 1H), 8.13 (d, J=9.1 Hz, 1H), 7.90 (d, J=9.1 Hz, 1H), 4.61 (s, 2H), 3.93 (t, J=5.8 Hz, 2H), 3.64 (d, J=6.0 Hz, 2H), 3.49 (d, J=6.1 Hz, 1H), 2.16 (s, 2H), 1.99 (s, 1H); ¹³C NMR (126 MHz, DMSO) δ 169.21, 140.24, 139.55, 139.27, 134.78, 132.70, 131.88, 128.81, 123.06, 122.63, 120.92, 115.44, 67.49, 42.57, 42.23, 30.01, 25.60. HRMS (ESI) m/z calcd for C₁₉H₁₆F₃N₆O [M+H]⁺ 401.1338, found 401.1328.

### 9-Cyclopropyl-7-(3-(trifluoromethyl)-1H-pyrazol4-yl)-3H-pyrazolo[4,3-f]quinolone

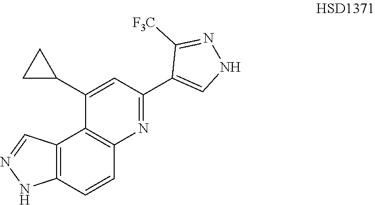 [SMILES: FC(F)(F)c1n[nH]cc1-c1cc(C2CC2)c2c(ccc3[nH]ncc32)n1]

Method A: Off-white solid (124 mg, 36%). ¹H NMR (500 MHz, DMSO-d₆) δ 8.80 (s, 1H), 8.66 (s, 1H), 7.91 (dd, J=9.1, 2.8 Hz, 1H), 7.80 (d, J=9.1 Hz, 1H), 7.62 (d, J=2.8 Hz, 1H), 2.69-2.53 (m, 1H), 1.28 (dq, J=9.3, 4.0, 3.2 Hz, 2H), 0.94 (di, J=5.2, 2.4 Hz, 2H); ¹³C NMR (126 MHz, DMSO) δ 148.26, 147.74, 145.97, 145.51, 138.44, 131.97, 129.51, 123.71, 122.38, 121.62, 121.41, 118.34, 116.44, 115.57, 16.42, 7.59.

### 4-(3,8,9,10,11,12-Hexahydrocyclohepta[c]pyrazolo[4,3-f]quinolin-7-yl)-2,6-diiodophenol

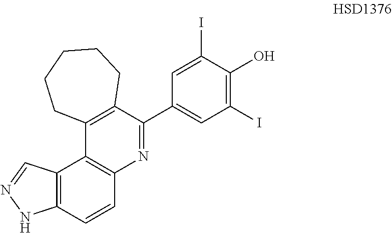 [SMILES: Oc1c(I)cc(-c2nc3ccc4[nH]ncc4c3c3c2CCCCC3)cc1I]

Method A: Yellow solid (203 mg, 35%). ¹H NMR (500 MHz, DMSO-d₆) δ 9.92 (s, 1H), 8.72 (d, J=17.9 Hz, 1H), 8.00 (d, J=9.1 Hz, 1H), 7.93-7.84 (m, 3H), 3.61-3.52 (m, 2H), 3.04-2.96 (m, 2H), 1.97-1.81 (m, 4H), 1.70-1.59 (m, 2H); ¹³C NMR (126 MHz, DMSO) δ 156.48, 150.93, 145.52, 141.82, 140.39, 137.85, 136.37, 133.12, 128.72, 121.97, 115.53, 87.03, 31.53, 31.18, 30.00, 26.90, 24.65. HRMS (ESI) m/z calcd for C₂₁H₁₈I₁₂N₃O [M+H]⁺581.9539, found 581.9544.

### 7-(4-Hydroxy-3,5-diiodophenyl)-8,9,10,11-tetrahydro-3H-pyrazolo[4,3-a]phenanthridin-9-ol

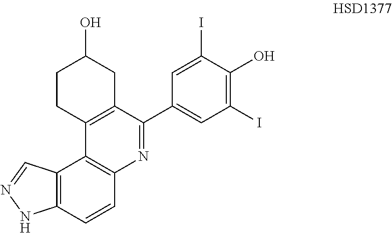 [SMILES: Oc1c(I)cc(-c2nc3ccc4[nH]ncc4c3c3c2CC(O)CC3)cc1I]

Method A: Yellow solid (175 mg, 30%). ¹H NMR (500 MHz, DMSO-d₆) δ 10.20 (s, 1H), 8.80 (s, 1H), 8.22 (d, J=9.3 Hz, 1H), 8.16 (d, J=9.1 Hz, 1H), 8.12 (s, 2H), 4.07 (dq, J=10.8, 6.9, 4.9 Hz, 1H), 3.56 (dt, J=19.1, 6.2 Hz, 1H), 3.50-3.46 (m, 1H), 2.99 (dd, J=16.5, 4.1 Hz, 1H), 2.75 (dd, J=16.5, 6.7 Hz, 1H), 2.20-2.09 (m, 1H), 2.07-1.96 (m, 1H); ¹³C NMR (126 MHz, DMSO) δ 157.81, 154.46, 148.61, 140.62, 136.28, 135.37, 130.03, 122.73, 114.93, 87.09, 63.37, 36.50, 29.26, 28.51. HRMS (ESI) m/z calcd for C₂₀H₁₆I₂N₃O₂ [M+H]⁺ 583.9332, found 583.9336.

### 2,6-Dibromo-4-(8,9,10,11-tetrahydro-3H-pyrazolo[4,3-a]phenanthridin-7-yl)phenol

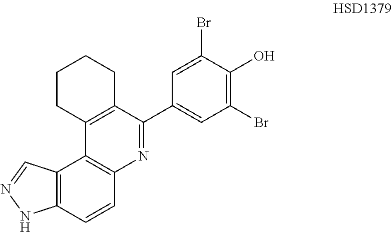 [SMILES: Oc1c(Br)cc(-c2nc3ccc4[nH]ncc4c3c3c2CCCC3)cc1Br]

Method A: Off-white solid (113 mg, 24%). ¹H NMR (500 MHz, DMSO-d₄) δ 10.25 (s, 1H), 8.58 (s, 1H), 7.92 (d, J=9.1 Hz, 1H), 7.85 (d, J=9.1 Hz, 1H), 7.79 (s, 2H), 3.29 (d, J=6.6 Hz, 2H), 2.80 (t, J=6.1 Hz, 2H), 2.03-1.93 (m, 2H), 1.79-1.67 (m, 2H); ¹³C NMR (126 MHz, DMSO) δ 152.73, 151.40, 144.73, 142.09, 136.53, 133.76, 133.49, 129.93, 127.88, 122.31, 115.95, 111.95, 29.92, 28.66, 22.34. HRMS (ESI) m/z calcd for C₂₀H₁₆Br₂N₃O [M+H]⁺471.9660, found 471.9645.

### 7-(4-Hydroxy-3,5-diiodophenyl)-8,9,10,10-tetrahydro-3H-pyrazolo[4,3-a]phenanthridine-1-carbonitrile

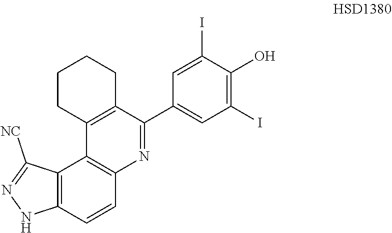 [SMILES: N#Cc1n[nH]c2ccc3nc(-c4cc(I)c(O)c(I)c4)c4c(c3c12)CCCC4]

Method A: Yellow solid (224 mg, 38%). ¹H NMR (500 MHz, DMSO-d₆) δ 9.77 (s, 1H), 7.93 (s, 2H), 7.86-7.77 (m, 2H), 3.40 (t, J=6.5 Hz, 2H), 2.77 (t, J=6.2 Hz, 2H), 1.90 (ddt, J=9.2, 6.5, 3.0 Hz, 2H), 1.75 (ddt, J=9.2, 6.4, 3.0 Hz, 2H); ¹³C NMR (126 MHz, DMSO) δ 155.88, 155.23, 144.40, 143.50, 140.00, 139.78, 136.45, 131.67, 129.81, 121.16, 120.30, 117.66, 116.23, 114.52, 86.91, 31.76, 28.66, 22.03, 21.81. HRMS (ESI) m/z calcd for C₂₂H₁₅I₂N₃O [M+H]⁺ 590.9304, found 590.9325.

### 2,6-Diiodo-4-(1-methyl-8,9,10,11-tetrahydro-3H-pyrazolo[4,3-a]phenanthridin-7-yl)phenol

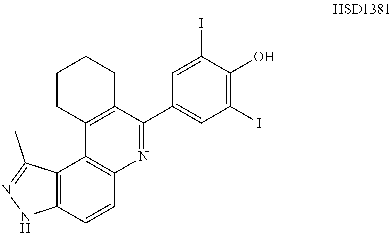 [SMILES: Cc1n[nH]c2ccc3nc(-c4cc(I)c(O)c(I)c4)c4c(c3c12)CCCC4]

Method A: Yellow solid (244 mg, 42%). ¹H NMR (500 MHz, DMSO-d₆) δ 10.08 (s, 1H), 8.04 (d, J=9.6 Hz, 3H), 7.95 (d, J=8.9 Hz, 1H), 3.61-3.50 (m, 2H), 2.92 (s, 3H), 2.82-2.71 (m, 2H), 1.87-1.74 (m, 4H), ¹³C NMR (126 MHz, DMSO) δ 157.30, 140.34, 130.74, 125.25, 87.10, 32.52, 27.67, 21.87, 21.55. HRMS (ESI) m/z calcd for C₂₁H₁₈I₂N₃O [M+H]⁺581.9539 found 581.9534.

### (5-(8,9,10,11-Tetrahydro-3H-pyrazolo[4,3-a]phenanthridin-7-yl)thiophen-3-yl)boronic acid

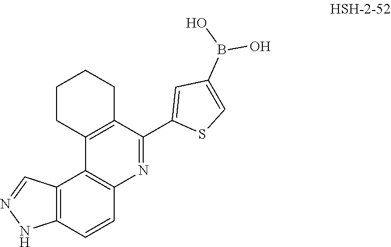 [SMILES: OB(O)c1csc(-c2nc3ccc4[nH]ncc4c3c3c2CCCC3)c1]

Method A: Off-white solid (157, 45%). ¹H NMR (500 MHz, DMSO-d₆) δ 8.54 (s, 1H), 8.08 (s, 2H), 8.05 (s, 1H), 7.91 (d, J=6.8 Hz, 1H), 7.84 (d, J=9.1 Hz, 1H), 7.78 (d, J=9.0 Hz, 1H), 3.11 (t, J=6.1 Hz, 2H), 2.46 (s, 2H), 2.06-1.98 (m, 2H), 1.88 (qd, J=8.6, 7.1, 3.9 Hz, 2H); ¹³C NMR (126 MHz, DMSO-d₆) δ 149.49, 145.18, 143.33, 143.01, 138.66, 137.70, 136.36, 133.33, 129.32, 128.70, 121.48, 116.41, 114.79, 30.05, 29.29, 22.62, 22.38.

### N-(2-(4-Methylpiperazin-1-yl)ethyl)-7-(3-(trifluoromethyl)-1H-pyrazol-4-yl)-8,9,10,11-tetrahydro-3H-pyrazolo[4,3-a]phenanthridine-1-carboxamide

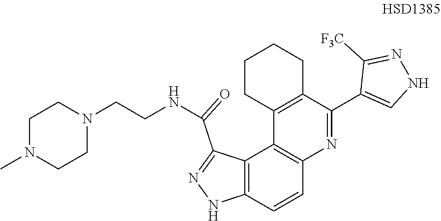 [SMILES: CN1CCN(CCNC(=O)c2n[nH]c3ccc4nc(-c5c[nH]nc5C(F)(F)F)c5c(c4c23)CCCC5)CC1]

Pale yellow solid. ¹H NMR (500 MHz, Methanol-d₄) δ 8.29 (s, 1H), 8.25 (d, J=9.2 Hz, 1H), 8.03 (d, J=9.2 Hz, 1H), 3.75 (t, J=6.4 Hz, 2H), 3.58 (t, J=6.0 Hz, 2H), 3.41 (s, 4H), 3.19-3.05 (m, 4H), 3.01-2.96 (m, 2H), 2.91 (s, 3H), 2.81 (t, J=6.5 Hz, 2H), 2.01-1.92 (m, 2H), 1.92-1.87 (m, 2H); ¹³C NMR (126 MHz, MeOD) δ 167.01, 154.73, 144.30, 143.47, 140.67, 136.64, 133.59, 132.09, 124.38, 122.36 (q, J=268.38 Hz), 121.24, 119.27, 111.50, 110.90, 55.67, 52.74, 49.61, 42.13, 36.36, 30.49, 26.83, 21.20, 20.97. HRMS (ESI) m/z calcd for C₂₆H₃₀F₃N₈O [M+H]⁺ 527.2495, found 527.2495.

### 1-Methyl-7-(3-(trifluoromethyl)-1H-pyrazol-4-yl)-8,9,10,11-tetrahydro-3H-pyrazolo[4,3-a]phenanthridin-9-amine

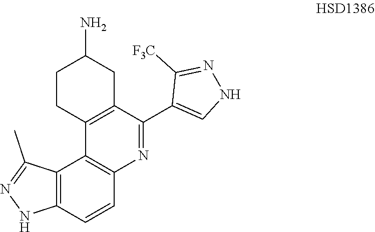 [SMILES: Cc1n[nH]c2ccc3nc(-c4c[nH]nc4C(F)(F)F)c4c(c3c12)CCC(N)C4]

Method A: Off-white solid (89 mg, 23%). ¹H NMR (500 MHz, DMSO-d₆) δ 8.55 (s, 2H), 8.44 (s, 1H), 8.10 (d, J=9.1 Hz, 1H), 8.02 (d, J=9.3 Hz, 1H), 3.82-3.71 (m, 2H), 3.68-3.59 (m, 1H), 3.14-3.04 (m, 1H), 2.94 (s, 3H), 2.90-2.79 (m, 1H), 2.42-2.28 (m, 1H), 1.91-1.78 (m, 1H); ¹³C NMR (126 MHz, DMSO) δ 144.18, 140.74, 139.47, 139.18, 138.89, 132.95, 129.06, 125.24, 122.97 (q, J=269.64 Hz), 113.03, 45.50, 31.39, 30.49, 26.00, 18.97. HRMS (ESI) m/z calcd for C₁₉H₁₈F₃N₆ [M+H]⁺387.1545, found 387.1540.

### (2-Methoxy-5-(8,9,10,11-tetrahydro-3H-pyrazolo[4,3-a]phenanthridin-7-yl)phenyl)boronic acid

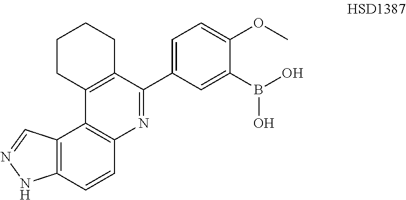 [SMILES: COc1ccc(-c2nc3ccc4[nH]ncc4c3c3c2CCCC3)cc1B(O)O]

Method A: White solid (166 mg, 48%). ¹H NMR (500 MHz, DMSO-d₆) δ 8.57-8.51 (m, 1H), 7.83 (s, 2H), 7.76 (d, J=6.0 Hz, 3H), 7.60 (dd, J=8.5, 2.4 Hz, 1H), 7.06 (d, J=8.5 Hz, 1H), 3.87 (s, 3H), 3.36-3.32 (m, 2H), 2.78 (t, J=6.1 Hz, 2H), 2.05-1.95 (m, 2H), 1.78-1.69 (m, 2H); ¹³C NMR (126 MHz, DMSO) δ 163.59, 156.90, 143.67, 142.28, 138.64, 136.72, 136.33, 133.31, 132.83, 129.70, 129.44, 121.61, 116.55, 114.27, 110.23, 55.95, 29.73, 29.20, 22.67, 22.62. HRMS (ESI) m/z calcd for C₂₁H₂₁ BN₃O₃[M+H]⁺ 374.1676, found 374.1671.

### (5-(8,9,10,11-Tetrahydro-3H-pyrazolo[4,3-a]phenanthridin-7-yl)furan-2-yl)boronic acid

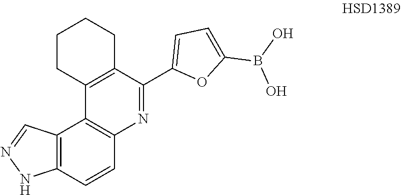 [SMILES: OB(O)c1ccc(-c2nc3ccc4[nH]ncc4c3c3c2CCCC3)o1]

Method A: Yellow solid (133 mg, 40%). ¹H NMR (500 MHz, DMSO-d₆) δ 8.64-8.60 (m, 1H), 8.02-7.94 (m, 2H), 7.31-7.21 (m, 1H), 6.79 (m, 1H), 3.35-3.34 (m, 2H), 3.07-3.03 (m, 2H), 2.00 (s, 2H), 1.86 (s, 2H); ¹³C NMR (126 MHz, DMSO) δ 154.27, 145.25, 129.18, 122.17, 116.19, 112.66, 30.33, 28.05, 22.07. HRMS (ESI) m/z calcd for C₁₈H₁₇BN₃O₃ [M+H]⁺ 334.1363, found 334.1363.

### 5-Fluoro-7-(1H-pyrazol-4-yl)-8,9,10,11-tetrahydro-3H-pyrazolo[4,3-a]phenanthridine

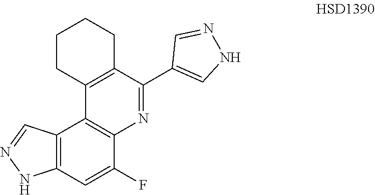 [SMILES: Fc1cc2[nH]ncc2c2c3c(c(-c4cn[nH]c4)nc12)CCCC3]

Method A: Off-white solid (64 mg, 21%). ¹H NMR (500 MHz, Methanol-d₄) δ 8.81 (s, 1H), 8.29 (s, 2H), 8.00 (d, J=10.3 Hz, 1H), 3.57 (t, J=6.4 Hz, 2H), 3.09 (t, J=6.2 Hz, 2H), 2.20-2.10 (m, 2H), 2.03-1.82 (m, 2H); ¹³C NMR (126 MHz, MeOD) δ 153.31, 146.28, 135.79, 133.97, 133.51, 126.93, 123.44, 112.98, 111.93, 103.01, 30.97, 27.96, 21.18. HRMS (ESI) m/z calcd for C₁₇H₁₅FN₅ [M+H]⁺ 308.1311, found 308.1312.

### (7-(3-(Trifluoromethyl)-1H-pyrazol-4-yl)-8,9,10,11-tetrahydro-3H-pyrazolo[4,3-a]phenanthridin-1-yl)methanol

Compound HSD1329 (100 mg) was dissolved in anhydrous THF and cooled on using dry ice acetone under argon. After that LAH (1 molar solution in THF, 1 equiv.) was added dropwise and reaction was continued to stir for overnight at room temperature. Reaction was quenched by slow addition of water and THF mixture slowly under cooling condition. Reaction mixture filtered and purified by flash column chromatography.

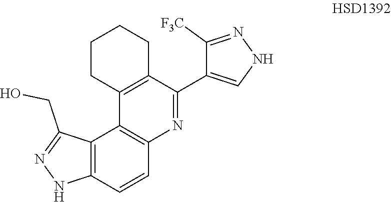 [SMILES: OCc1n[nH]c2ccc3nc(-c4c[nH]nc4C(F)(F)F)c4c(c3c12)CCCC4]

white solid (75 mg, 80%). ¹H NMR (500 MHz, Methanol-d₄) δ 7.98 (s, 1H), 7.79 (d, J=8.4 Hz, 2H), 5.24 (s, 2H), 3.70 (s, 2H), 2.71 (t, J=5.8 Hz, 2H), 2.00-1.84 (m, 4H); ¹³C NMR (126 MHz, MeOD) δ 148.12, 144.28, 143.66, 139.73, 133.60, 130.48, 130.17, 128.88, 124.42, 118.85, 114.15, 60.41, 29.11, 27.68, 22.06, 21.83. HRMS (ESI) m/z calcd for C₁₉H₁₇F₃N₅O [M+H]⁺ 388.1385, found 388.1380.

### 1-Methyl-7-(3-(trifluoromethyl)-1H-pyrazol-4-yl)-8,9,10,11-tetrahydro-3H-pyrazolo[4,3-a]phenanthridine-8,8,9,9,10,10,-d₆

Deuterated (D₈) cyclohexanone was used as a ketone using same method A

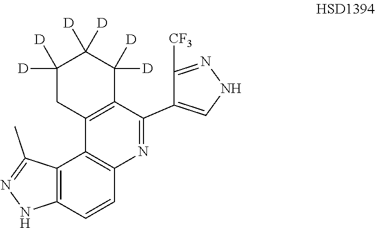 [SMILES: [2H]C1([2H])Cc2c(c(-c3c[nH]nc3C(F)(F)F)nc3ccc4[nH]nc(C)c4c23)C([2H])([2H])C1([2H])[2H]]

Method A: Off-white solid (68 mg, 18%). ¹H NMR (500 MHz, Methanol-d₄) δ 8.20 (s, 1H), 7.72 (d, J=8.9 Hz, 1H), 7.68 (d, J=8.7 Hz, 1H), 3.40-3.37 (m, 2H), 2.89 (s, 3H); HRMS (ESI) m/z calcd for C₁₉H₁₁D₆F₃N₅ [M+H]⁺ 378.1813, 378.1804.

### 5-Fluoro-7-(3-(trifluoromethyl)-1H-pyrazol-4-yl)-8,9,10,11-tetrahydro-3H-pyrazolo[4,3-a]phenanthridine-8,8,9,9,10,10,-d₆

Deuterated (D₈) cyclohexanone was used as a ketone using same method A

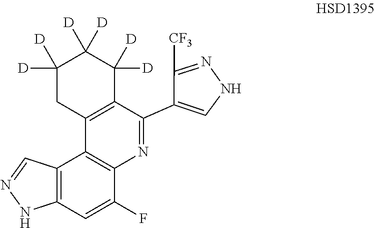 [SMILES: [2H]C1([2H])Cc2c(c(-c3c[nH]nc3C(F)(F)F)nc3c(F)cc4[nH]ncc4c23)C([2H])([2H])C1([2H])[2H]]

Method A: Off-white solid (76 mg, 20%). ¹H NMR (500 MHz, Methanol-d₄) δ 8.80 (s, 1H), 8.24 (s, 1H), 7.96 (d, J=9.9 Hz, 1H), 3.53 (s, 2H); ¹³C NMR (126 MHz, MeOD) δ 154.06 (q, J=255.78 Hz), 144.99, 140.18, 139.89, 139.23, 134.60, 134.02, 131.99, 128.78, 124.39, 122.45 (q, J=269.64 Hz), 118.14, 112.31, 111.91, 102.86, 30.30, 26.74, 20.13, 16.82. HRMS (ESI) m/z calcd for C₁₈H₈D₆F₄N₅[M+H]⁺ 382.1562 found 382.1556.

### 7-(3-(Trifluoromethyl)-1H-pyrazol-4-yl)-3H-pyrazolo[4,3-f]quinoline-9-carboxylic acid

Pyruvic acid as ketone substrate.

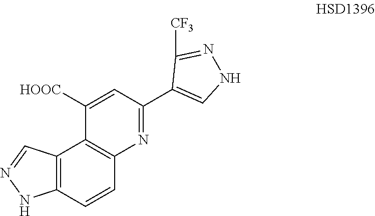 [SMILES: O=C(O)c1cc(-c2c[nH]nc2C(F)(F)F)nc2ccc3[nH]ncc3c12]

Method A: Pale yellow solid (100 mg, 29%). ¹H NMR (500 MHz, Methanol-d₄) δ 8.69 (s, 1H), 8.32 (s, 1H), 7.96-7.87 (m, 2H), 7.82 (s, 1H); ¹³C NMR (126 MHz, MeOD) δ 173.72, 147.71, 146.35, 143.54, 139.55, 138.93, 134.30, 131.27, 128.76, 122.98 (q, J=269.64 Hz), 120.75, 118.19, 117.21, 116.10, 115.45.

### 3,3-Dimethyl-5-(8,9,10,11-tetrahydro-3H-pyrazolo[4,3-a]phenanthridin-7-yl)benzo[c][1,2]oxaborol-1(3H)-ol

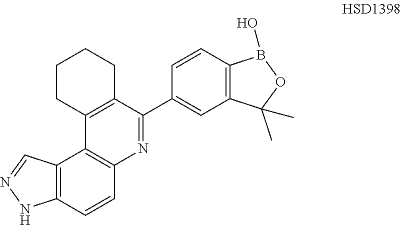 [SMILES: CC1(C)OB(O)c2ccc(-c3nc4ccc5[nH]ncc5c4c4c3CCCC4)cc21]

Method A: Off-white solid (54 mg, 14%). ¹H NMR (500 MHz, Methanol-d₄) δ 7.83 (d, J=9.1 Hz, 1H), 7.77 (d, J=9.1 Hz, 1H), 7.73-7.68 (m, 1H), 7.47 (t, J=1.1 Hz, 1H), 7.41 (dd, J=7.4, 1.4 Hz, 1H), 3.24 (t, J=6.5 Hz, 2H), 2.69 (t, J=6.2 Hz, 2H), 2.05-1.94 (m, 2H), 1.79-1.71 (m, 2H), 1.56 (s, 6H); ¹³C NMR (126 MHz, MeOD) δ 162.29, 157.20, 143.50, 143.12, 129.88, 129.44, 128.35, 127.81, 122.15, 120.98, 119.24, 119.15, 115.90, 110.35, 98.72, 84.23, 29.51, 29.30, 28.40, 28.29, 22.07, 21.97; HRMS (ESI) m/z calcd for C₂₃H₂₃BN₃O₂ [M+H]⁺ 384.1883 found 384.1882.

### 3,3-Dimethyl-6-(8,9,10,11-tetrahydro-3H-pyrazolo[4,3-a]phenanthridin-7-yl)benzo[c][1,2]oxaborol-1(3H)-ol

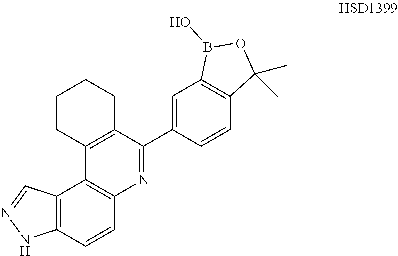 [SMILES: CC1(C)OB(O)c2cc(-c3nc4ccc5[nH]ncc5c4c4c3CCCC4)ccc21]

Method A: Off-white solid (62 mg, 16%). ¹H NMR (500 MHz, Methanol-d₄) δ 8.62 (s, 1H), 7.88 (dd, J=17.5, 9.0 Hz, 2H), 7.79-7.68 (m, 1H), 7.65-7.59 (m, 1H), 7.55-7.46 (m, 1H), 3.53-3.40 (m, 2H), 2.93-2.72 (m, 2H), 2.11 (dt, J=11.5, 6.6 Hz, 2H), 1.94-1.76 (m, 2H), 1.60 (s, 6H); ¹³C NMR (126 MHz, MeOD) δ 162.13, 157.52, 154.20, 143.64, 139.57, 138.67, 131.57, 130.39, 129.75, 128.47, 122.21, 120.28, 113.77, 84.16, 29.64, 28.56, 28.27, 22.16, 22.05. HRMS (ESI) m/z calcd for C₂₃H₂BN₃O₂ [M+H]⁺ 384.1883 found 384.1880.

### (2-Methoxy-3-(8,9,10,11-tetrahydro-3H-pyrazolo[4,3-a]phenanthridin-7-yl)phenyl)boronic acid

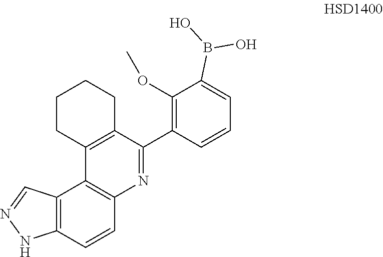 [SMILES: COc1c(B(O)O)cccc1-c1nc2ccc3[nH]ncc3c2c2c1CCCC2]

Method A: Off-white solid (41 ng, 11%). ¹H NMR (500 MHz, Methanol-d₄) δ 8.58 (s, 11H), 7.89 (d, J=9.2 Hz, 1H), 7.83 (d, J=9.1 Hz, 1H), 7.42 (d, J=7.3 Hz, 1H), 7.32 (d, J=7.4 Hz, 1H), 7.24 (t, J=7.4 Hz, 1H), 3.48 (s, 3H), 3.44-3.35 (m, 2H), 2.82 (dd, J=15.9, 7.2 Hz, 1H), 2.51 (dt, J=17.3, 5.9 Hz, 1H), 2.09-2.00 (m, 2H), 1.89-1.82 (m, 1H), 1.81-1.76 (m, 1H); ¹³C NMR (126 MHz, MeOD) δ 159.79, 155.68, 142.96, 133.21, 132.37, 131.13, 130.91, 128.28, 124.23, 123.00, 122.24, 60.00, 29.48, 27.03, 22.16, 21.79. HRMS (ESI) m/z calcd for C₂₁H₂₁BN₃O₃ [M+H]⁺ 374.1676, found 374.1670.

### (7-(3-(Trifluoromethyl)-1H-pyrazol-4-yl)-8,9,10,11-tetrahydro-3H-pyrazolo[4,3-a]phenanthridin-5-yl)ethane-1,2-diamine

N¹, N¹, N², Trimethylethane-1,2-diamine (0.5 mL) was added to mixture of compound HSH-1-156 (50 mg, 0.13 mmol) and powdered K₂CO₃ (2 equiv), dissolved in DMSO (1 mL). The reaction mixture was heated at 200° C. in a pressure tube for 24 h. After completion of reaction, reaction was purified by silica-gel flash chromatography using dichloromethane:methanol (80:20) as solvent system.

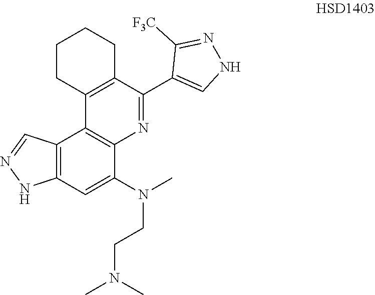 [SMILES: CN(C)CCN(C)c1cc2[nH]ncc2c2c3c(c(-c4c[nH]nc4C(F)(F)F)nc12)CCCC3]

Yellow solid (8 mg, 13%). ¹H NMR (500 MHz, Methanol-d₄) δ 8.51 (s, 1H), 8.07 (s, 1H), 7.45 (s, 1H), 3.52 (t, J=6.4 Hz, 2H), 3.37 (t, J=6.4 Hz, 2H), 3.21-3.16 (m, 2H), 2.98 (s, 3H), 2.66 (t, J=6.6 Hz, 2H), 2.43 (s, 6H), 2.10-2.04 (m, 2H), 1.92-1.84 (m, 2H); ¹³C NMR (126 MHz, MeOD) δ 148.52, 146.67, 143.98, 139.58, 138.84, 131.49, 131.11, 123.95, 122.86, 120.72, 119.24, 112.05, 56.25, 52.86, 44.02, 43.71, 39.62, 29.85, 27.71, 22.14, 21.75; HRMS (ESI) m/z calcd for C₂₃H₂₄F₃N₇ [M+H]⁺ 458.2280, found 458.2276.

### 5-(Piperidin-1-yl)-7-(3-(trifluoromethyl)-1H-pyrazol-4-yl)-8,9,10,11-tetrahydro-3H-pyrazolo[4,3-a]phenanthridine

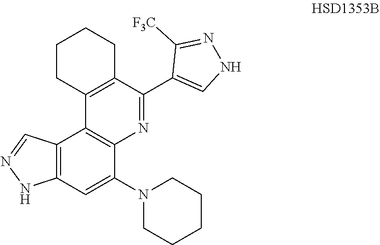 [SMILES: FC(F)(F)c1n[nH]cc1-c1nc2c(N3CCCCC3)cc3[nH]ncc3c2c2c1CCCC2]

Method A: Off-white solid (66 mg, 15%). ¹H NMR (500 MHz, DMSO-d₆) δ 8.39 (s, 1H), 8.15 (s, 1H), 7.12 (s, 1H), 3.24 (d, J=6.4 Hz, 2H), 3.17 (t, J=5.2 Hz, 4H), 2.62 (t, J=6.2 Hz, 2H), 1.93 (tt, J=7.6, 5.2, 4.3 Hz, 2H), 1.80-1.72 (m, 2H), 1.64 (t, J=5.8 Hz, 4H), 1.53 (q, J=6.1 Hz, 2H); ¹³C NMR (126 MHz, DMSO) δ 150.46, 146.47, 142.38, 139.11, 136.28, 131.34, 130.26, 123.57 (q, J=263.34 Hz), 123.19, 120.16, 120.08, 111.84, 53.57, 31.18, 29.81, 28.19, 26.16, 24.64, 22.68, 22.29; HRMS (ESI) m/z calcd for C₂₃H₂₄F₃N₆ [M+H]⁺ 441.2015. found 441.2012.

### 7-(4-Fluoro-3-nitrophenyl)-8,9,10,11-tetrahydro-3H-pyrazolo[4,3-a]phenanthridine

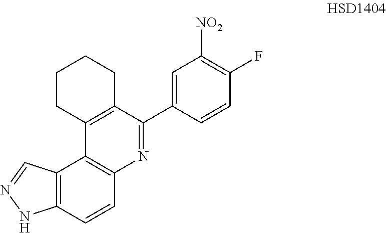 [SMILES: O=[N+]([O-])c1cc(-c2nc3ccc4[nH]ncc4c3c3c2CCCC3)ccc1F]

Method A: Yellow solid (145 mg, 40%). ¹H NMR (500 MHz, Methanol-d₄) δ 8.63 (s, 1H), 8.31 (dd, J=7.2, 2.2 Hz, 1H), 7.96 (ddd, J=8.6, 4.3, 2.3 Hz, 1H), 7.93-7.87 (m, 2H), 7.58 (dd, J=11.0, 8.6 Hz, 1H), 3.44 (d, J=7.2 Hz, 3H), 2.84 (t, J=6.2 Hz, 2H), 2.18-2.08 (m, 2H), 1.94-1.83 (m, 2H); ¹³C NMR (126 MHz, MeOD) δ 153.80, 144.17, 137.50, 137.15, 136.31, 129.49, 128.62, 128.52, 126.50, 125.92, 122.66, 118.07, 117.90, 29.65, 28.38, 22.02.

### 2-Fluoro-5-(8,9,10,11-tetrahydro-3H-naphtho[1,2-e]indazol-7-yl)aniline

Compound HSD1404 (100 mg) was dissolved in 5 mL THF and purged with argon for 15 minute. After that palladium (10% on carbon, 20 mol %) was added and hydrogen ballon was used for hydrogenation for 6 h. After completion, reaction mixture was filtered through sintered funnel, concentrated and purified by flash column chromatography.

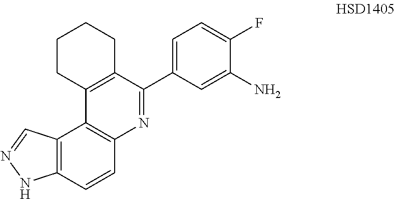 [SMILES: Nc1cc(-c2nc3ccc4[nH]ncc4c3c3c2CCCC3)ccc1F]

Pale yellow solid (83 mg, 90%). ¹H NMR (500 MHz, Methanol-d₄) δ 8.55 (s, 1H), 7.90-7.78 (m, 2H), 7.07 (dd, J=11.3, 8.2 Hz, 1H), 6.96 (dd, J=8.6, 2.1 Hz, 1H), 6.74 (ddd, J=8.2, 4.3, 2.1 Hz, 1H), 3.34 (d, J=7.9 Hz, 3H), 2.77 (t, J=6.2 Hz, 2H), 2.06 (td, J=8.6, 7.2, 4.6 Hz, 2H), 1.88-1.78 (m, 2H); ¹³C NMR (126 MHz, MeOD) δ 157.17, 153.73, 152.53 (d, J=239.4 Hz) 143.38, 136.87, 135.81, 135.63, 135.52, 129.70, 128.38, 122.05, 117.94, 117.38, 116.11, 114.39, 114.24, 113.58, 29.59, 28.41, 22.17, 22.06. HRMS (ESI) m/z calcd for C₂₁H₁₉FN₃ [M+H]⁺ 332.1563, found 332.1560.

### 7-(3-Fluoro-4-nitrophenyl)-8,9,10,11-tetrahydro-3H-pyrazolo[4,3-a]phenanthridine

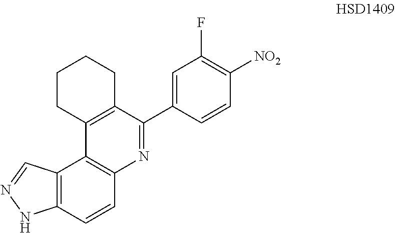 [SMILES: O=[N+]([O-])c1ccc(-c2nc3ccc4[nH]ncc4c3c3c2CCCC3)cc1F]

Method A: Yellow solid (145 mg, 40%) ¹H NMR (500 MHz, DMSO-d₆) δ 8.57 (s, 1H), 8.26 (t, J=8.2 Hz), 7.89-7.81 (m, 3H), 7.66 (dd, J=8.4, 1.4 Hz, 1H), 3.32 (s, 2H), 2.81 (t, J=5.8 Hz, 2H), 2.01-2.79 (m, 2H), 1.76-1.74 (m, 2H); ¹³C NMR (126 MHz, DMSO) δ 155.83, 153.75, 153.45, 149.27, 149.20, 143.60, 143.07, 139.01, 136.56, 129.52, 126.51, 122.60, 119.60, 119.43, 116.28, 114.94, 29.66, 228.50, 22.45, 22.33.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCES

1) Ten things you should know about protein kinases: IUPHAR Review 14. Fabbro et al. Br J Pharmacol 2015, 172, 2675.
2) 25 years of small molecular weight kinase inhibitors: potentials and limitations. Fabbro Mol Pharmacol 2015, 87, 766.
3) Bcr-Abl kinase domain mutations, drug resistance, and the road to a cure for chronic myeloid leukemia O'Hare et al. Blood 2007 110, 2242-2249.
4) Aberrant activation of the PI3K/mTOR pathway promotes resistance to sorafenib in AML. Lindblad et al. Oncogene 2016, 35 (39), 5119-31
5) Rho kinase regulates the survival and transformation of cells bearing oncogenic forms of KIT, FLT3, and BCR-ABL. Mali et al. Cancer Cell 2011, 20 (3), 357-69.
6) Jakinibs: a new class of kinase inhibitors in cancer and autoimmune disease. Kontzias et al. Curr. Opin. Pharmacol. 2012, 12 (4), 460-70.
7) Rho Kinase (ROCK) Inhibitors. James et al. J Cardiovasc Pharmacol. 2007, 50 (1), 17-24
8) An emerging treatment option for glaucoma: Rho kinase inhibitors. Wang and Chang Clin Ophthalmol. 2014, 8, 883-90.
9) LRRK2 in Parkinson's disease: protein domains and functional insights. Mata et al. Trends Neurosci. 2006, 29 (5), 286-93
10) Recent synthetic developments in a powerful imino Diels-Alder reaction (Povarov reaction): application to the synthesis of N-polyheterocycles and related alkaloids. Kouznctsov Tetrahedron, 2009, 65, 2721; b) Povarov and Mikhailov Izr Akad Nauk SSR, Ser Khim 1963, 953.

1. A compound having a formula 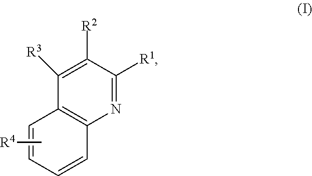
or a pharmaceutically acceptable salt thereof, wherein
R¹ is amino, hydroxyl, and derivatives thereof, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted;
R² and R³ are each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted;
or R² and R³ are taken together with the attached carbons to form an optionally substituted cyclic or heterocyclic moiety; and
R⁴ represents four substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; or any two adjacent substituents of the four substituents are taken together with the attached carbons form an optionally substituted cyclic or heterocyclic moiety.

2. The compound according to claim 1, wherein the compound has the following formula: 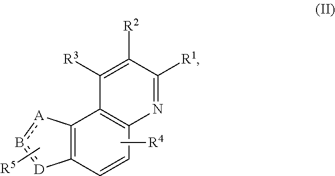
or a pharmaceutically acceptable salt thereof, wherein n=1˜5; the bonding between A and B, between B and D may be a double bond or a single bond, but cannot be double bond at the same time; A, B, and D represents, independently, C, O, N, and S wherein at least one of A, B, and D is a heteroatom;
R¹ is amino, hydroxyl, and derivatives thereof, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted;
R² and R³ are each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted;
or R² and R³ are taken together with the attached carbons to form an optionally substituted cyclic or heterocyclic moiety;
R⁴ represents two substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted;
or the two substituents are taken together with the attached carbons form an optionally substituted cyclic or heterocyclic moiety; and
depending on the element of A, B and D, R⁵ represents two or three substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted.

3. The compound according to claim 2, wherein R¹ is

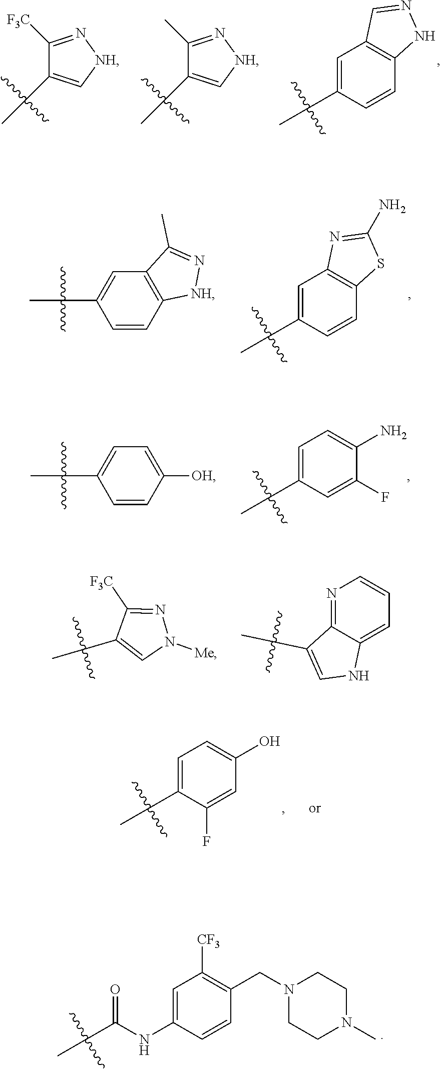

4. The compound according to claim 1, wherein the compound has the following formula: 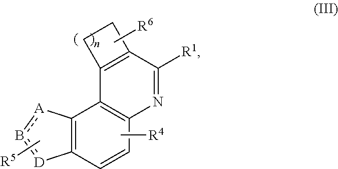
or a pharmaceutically acceptable salt thereof, wherein n=1˜5; the bonding between A and B, between B and D may be a double bond or a single bond, but cannot be double bond at the same time; A, B, and D represents, independently, C, O, N, and S wherein at least one of A, B, and D is a heteroatom;
R¹ is amino, hydroxyl, and derivatives thereof, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted;
R⁴ represents two substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted;
or the two substituents are taken together with the attached carbons form an optionally substituted cyclic or heterocyclic moiety,
depending on the element of A, B and D, R⁵ represents two or three substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; and
depending on the value of n, R⁶ represents two to six substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted.

5. The compound according to claim 4, wherein R¹ is

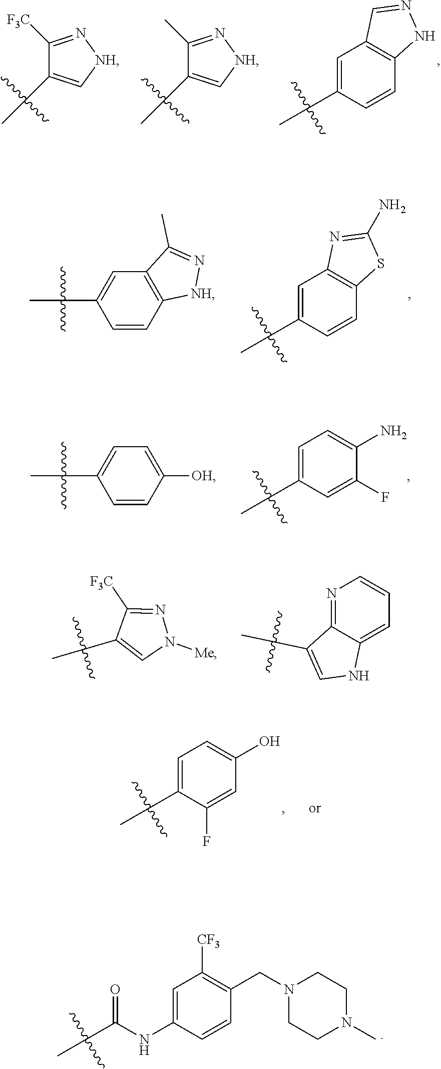

6. The compound according to claim 1, wherein the compound has the following formula: 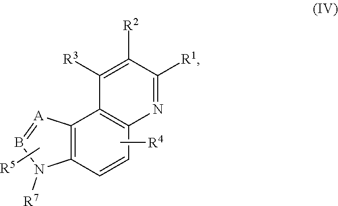
or a pharmaceutically acceptable salt thereof, wherein n=1˜5;
R¹ is amino, hydroxyl, and derivatives thereof, an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted;
R² and R³ are each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted;
or R² and R³ are taken together with the attached carbons to form an optionally substituted cyclic or heterocyclic moiety;
R⁴ represents two substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted;
or the two substituents are taken together with the attached carbons form an optionally substituted cyclic or heterocyclic moiety:
depending on the element of A and B specified below, R⁵ represents one or two substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted;
A and B represents, independently, CR⁸, N, or NR⁹, wherein R⁸ and R⁹ represent independently hydrogen, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted;
depending on the value of n, R⁶ represents two to six substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted; and
R⁷ is an alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, acyl, aryl, heteroaryl, arylalkyl, arylalkenyl, or arylalkynyl, each of which is optionally substituted.

7. The compound according to claim 6, wherein R¹ is

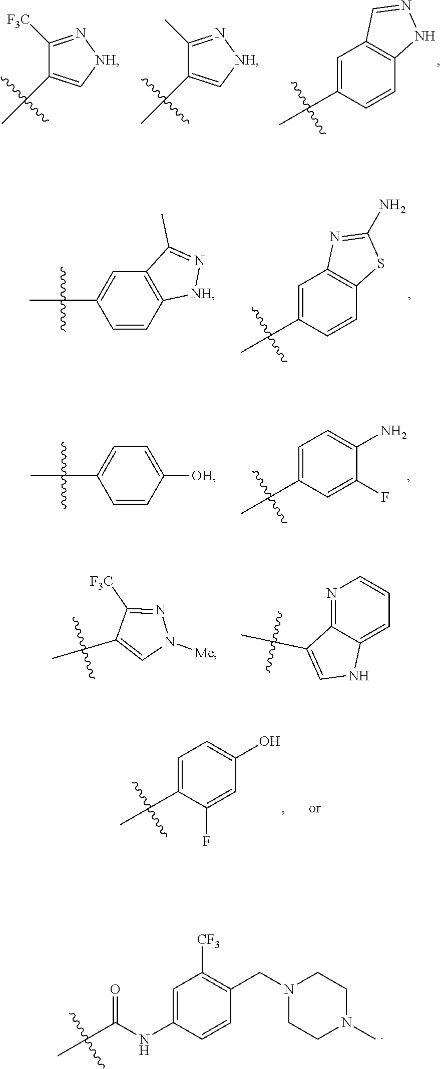

8. The compound according to claim 7, wherein R¹ is

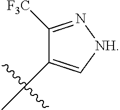 [SMILES: *c1c[nH]nc1C(F)(F)F]

9. The compound according to claim 6, wherein, n=3; R¹ is 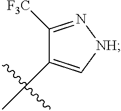 [SMILES: *c1c[nH]nc1C(F)(F)F]
A is carbon (C); B is nitrogen (N); R⁵, R⁶, and R⁷ all represent hydrogen, and R⁴ represents two substituents, each independently selected from the group consisting of hydrogen, deuterium, halo, azido, cyano, nitro, hydroxy, amino, thio, carboxy, ester, amide, and derivatives thereof, and acyl, sulfoxyl, sulfonyl, phosphate, phosphoryl, alkyl, alkenyl, alkynyl, heteroalkyl, heteroalkenyl, heteroalkynyl, heterocyclyl, cycloalkyl, cycloalkenyl, cycloheteroalkyl, cycloheteroalkenyl, aryl, heteroaryl, arylalkyl, arylalkenyl, and arylalkynyl, each of which is optionally substituted;
or the two substituents arm taken together with the attached carbons form an optionally substituted cyclic or heterocyclic moiety.

10. The compound according to claim 6, wherein R³ is

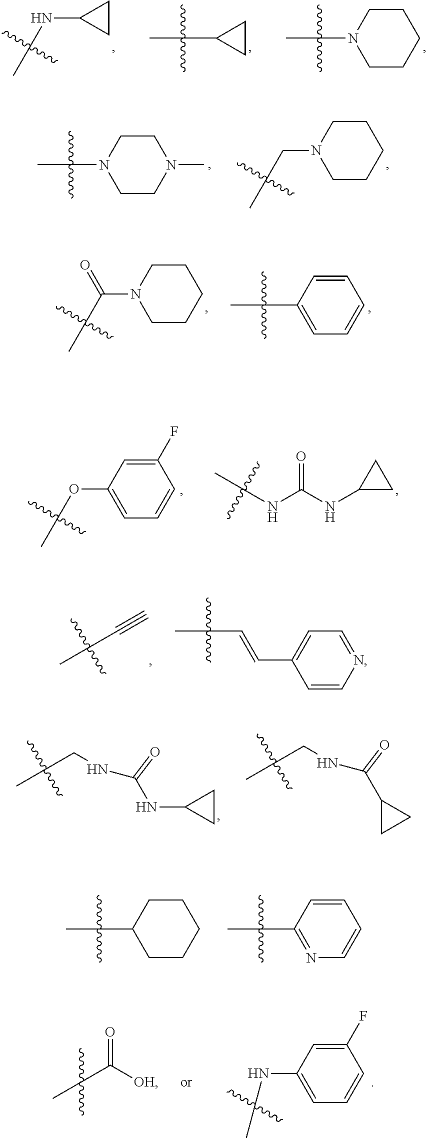

11. The compound according to claim 6, wherein R¹ is 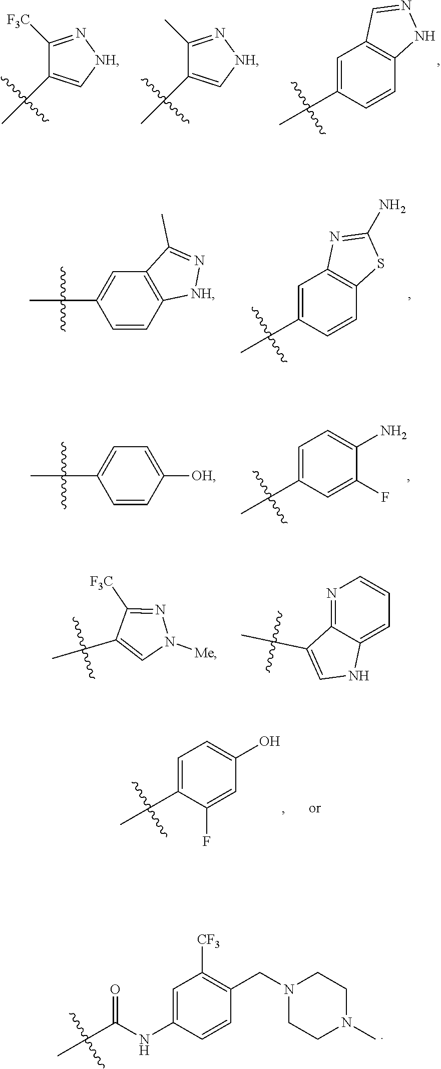
and
R³ is

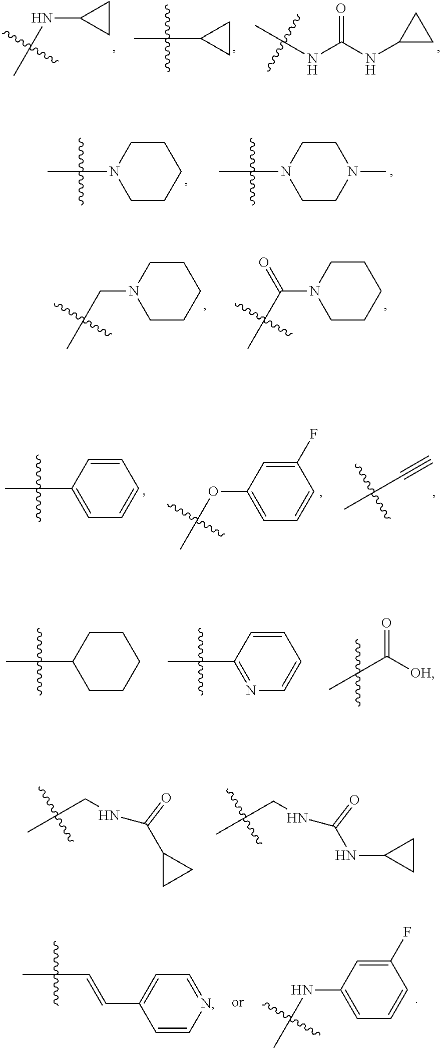

12. The compound according to claim 1, wherein the compound is

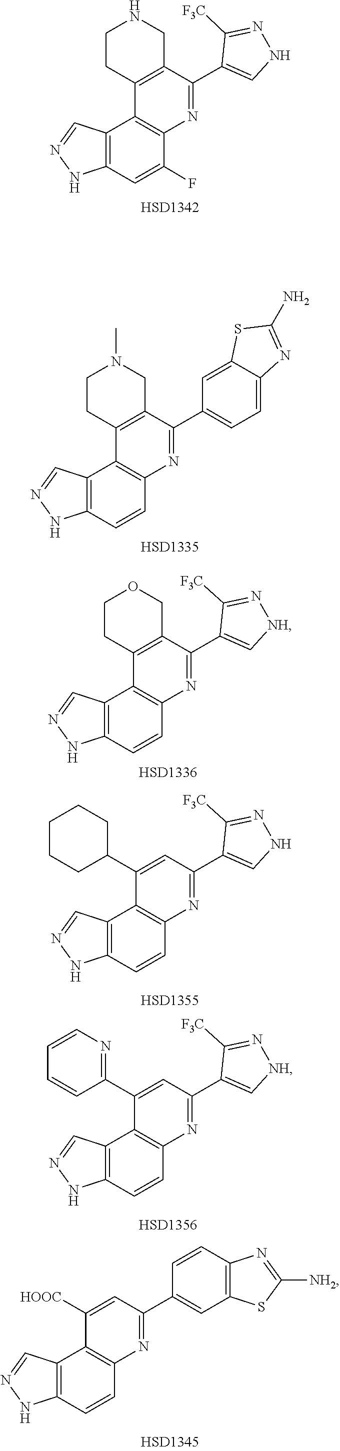

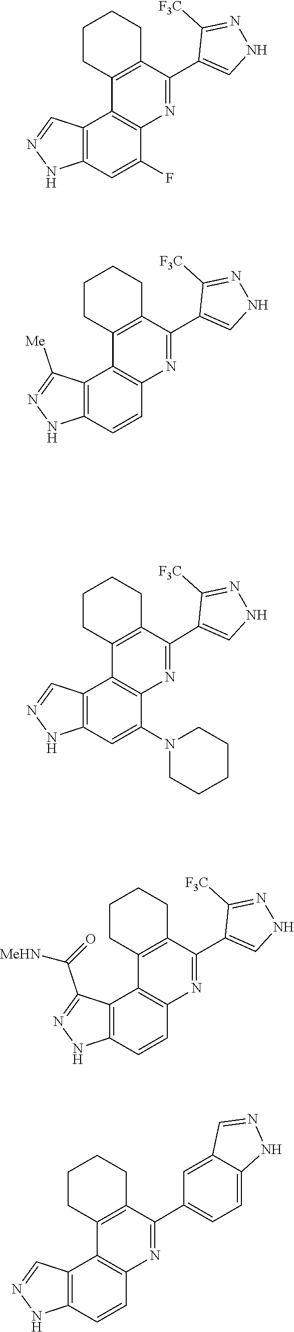 [SMILES: Fc1cc2[nH]ncc2c2c3c(c(-c4c[nH]nc4C(F)(F)F)nc12)CCCC3 ; Cc1n[nH]c2ccc3nc(-c4c[nH]nc4C(F)(F)F)c4c(c3c12)CCCC4 ; FC(F)(F)c1n[nH]cc1-c1nc2c(N3CCCCC3)cc3[nH]ncc3c2c2c1CCCC2 ; CNC(=O)c1n[nH]c2ccc3nc(-c4c[nH]nc4C(F)(F)F)c4c(c3c12)CCCC4 ; c1cc2[nH]ncc2cc1-c1nc2ccc3[nH]ncc3c2c2c1CCCC2]

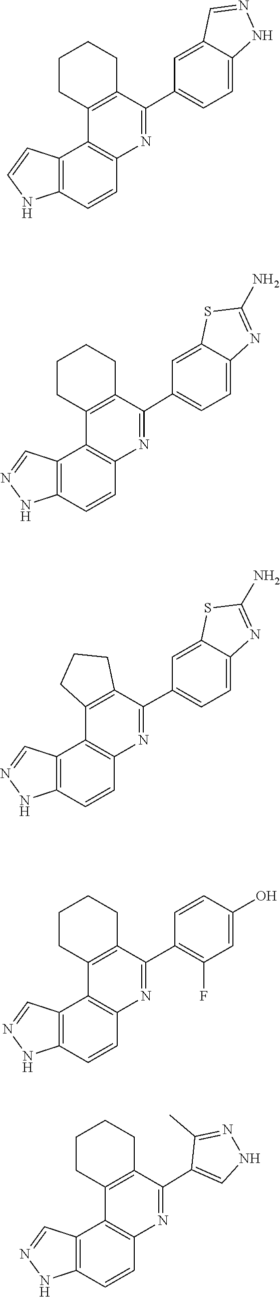 [SMILES: c1cc2c(ccc3nc(-c4ccc5[nH]ncc5c4)c4c(c32)CCCC4)[nH]1 ; Nc1nc2ccc(-c3nc4ccc5[nH]ncc5c4c4c3CCCC4)cc2s1 ; Nc1nc2ccc(-c3nc4ccc5[nH]ncc5c4c4c3CCC4)cc2s1 ; Oc1ccc(-c2nc3ccc4[nH]ncc4c3c3c2CCCC3)c(F)c1 ; Cc1n[nH]cc1-c1nc2ccc3[nH]ncc3c2c2c1CCCC2]

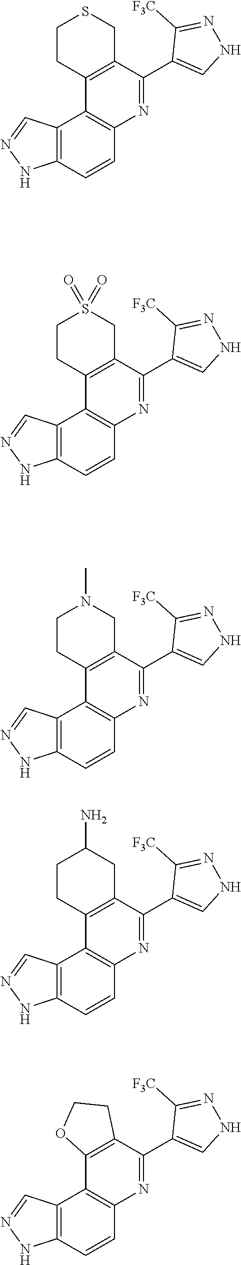

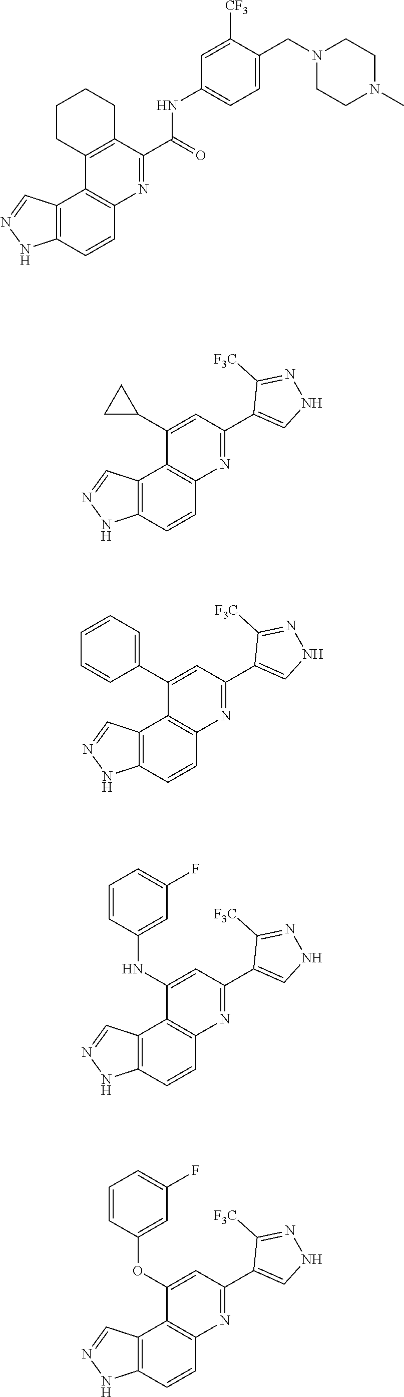 [SMILES: CN1CCN(Cc2ccc(NC(=O)c3nc4ccc5[nH]ncc5c4c4c3CCCC4)cc2C(F)(F)F)CC1 ; FC(F)(F)c1n[nH]cc1-c1cc(C2CC2)c2c(ccc3[nH]ncc32)n1 ; FC(F)(F)c1n[nH]cc1-c1cc(-c2ccccc2)c2c(ccc3[nH]ncc32)n1 ; Fc1cccc(Nc2cc(-c3c[nH]nc3C(F)(F)F)nc3ccc4[nH]ncc4c23)c1 ; Fc1cccc(Oc2cc(-c3c[nH]nc3C(F)(F)F)nc3ccc4[nH]ncc4c23)c1]

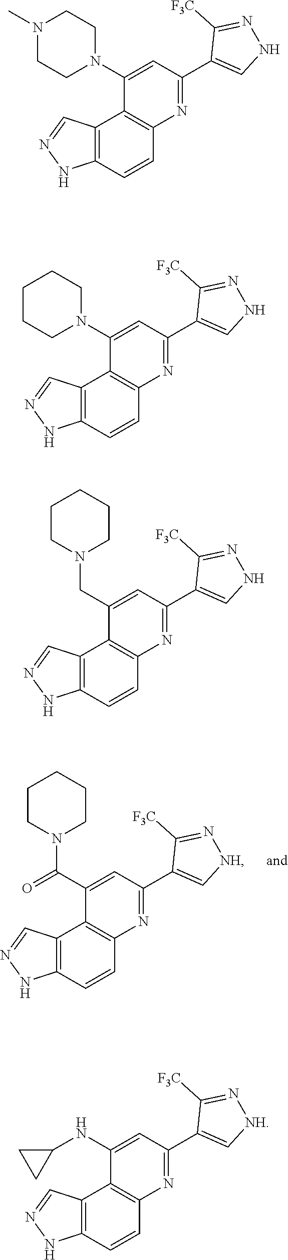 [SMILES: CN1CCN(c2cc(-c3c[nH]nc3C(F)(F)F)nc3ccc4[nH]ncc4c23)CC1 ; FC(F)(F)c1n[nH]cc1-c1cc(N2CCCCC2)c2c(ccc3[nH]ncc32)n1 ; FC(F)(F)c1n[nH]cc1-c1cc(CN2CCCCC2)c2c(ccc3[nH]ncc32)n1 ; O=C(c1cc(-c2c[nH]nc2C(F)(F)F)nc2ccc3[nH]ncc3c12)N1CCCCC1 ; FC(F)(F)c1n[nH]cc1-c1cc(NC2CC2)c2c(ccc3[nH]ncc32)n1]

13. The compound according to claim 1, wherein the compound is

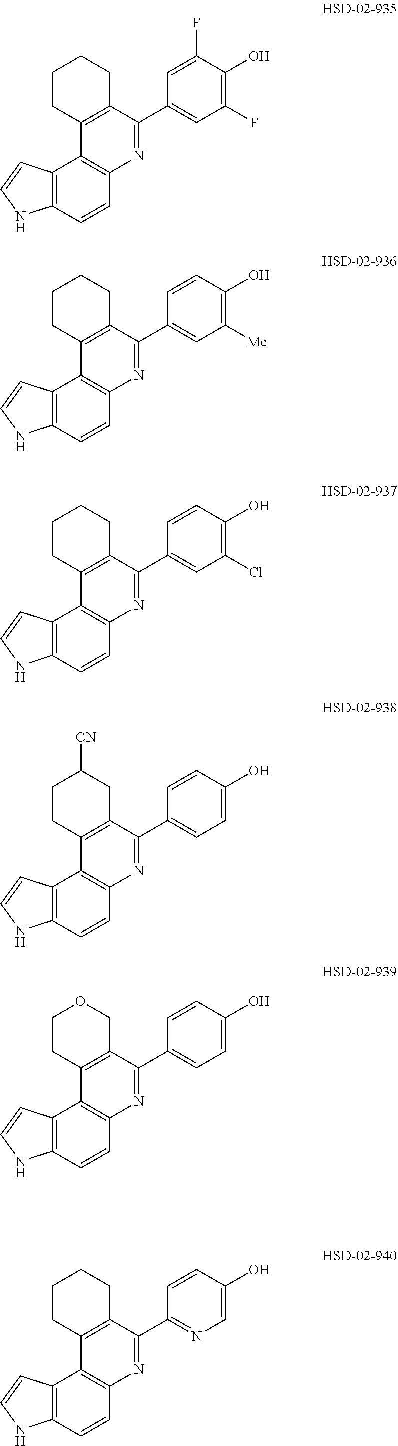 [SMILES: Oc1c(F)cc(-c2nc3ccc4[nH]ccc4c3c3c2CCCC3)cc1F ; Cc1cc(-c2nc3ccc4[nH]ccc4c3c3c2CCCC3)ccc1O ; Oc1ccc(-c2nc3ccc4[nH]ccc4c3c3c2CCCC3)cc1Cl ; N#CC1CCc2c(c(-c3ccc(O)cc3)nc3ccc4[nH]ccc4c23)C1 ; Oc1ccc(-c2nc3ccc4[nH]ccc4c3c3c2COCC3)cc1 ; Oc1ccc(-c2nc3ccc4[nH]ccc4c3c3c2CCCC3)nc1]

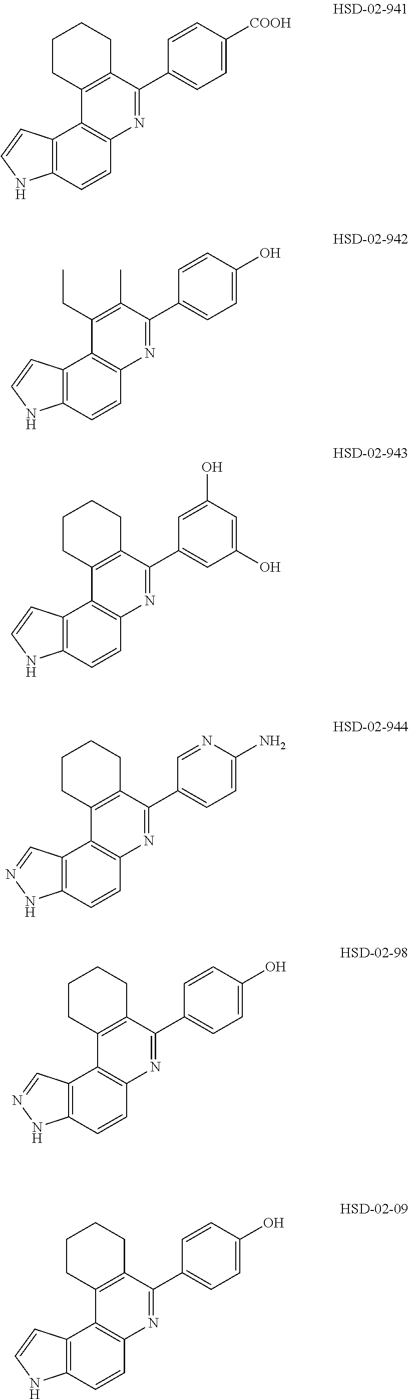 [SMILES: O=C(O)c1ccc(-c2nc3ccc4[nH]ccc4c3c3c2CCCC3)cc1 ; CCc1c(C)c(-c2ccc(O)cc2)nc2ccc3[nH]ccc3c12 ; Oc1cc(O)cc(-c2nc3ccc4[nH]ccc4c3c3c2CCCC3)c1 ; Nc1ccc(-c2nc3ccc4[nH]ncc4c3c3c2CCCC3)cn1 ; Oc1ccc(-c2nc3ccc4[nH]ncc4c3c3c2CCCC3)cc1 ; Oc1ccc(-c2nc3ccc4[nH]ccc4c3c3c2CCCC3)cc1]

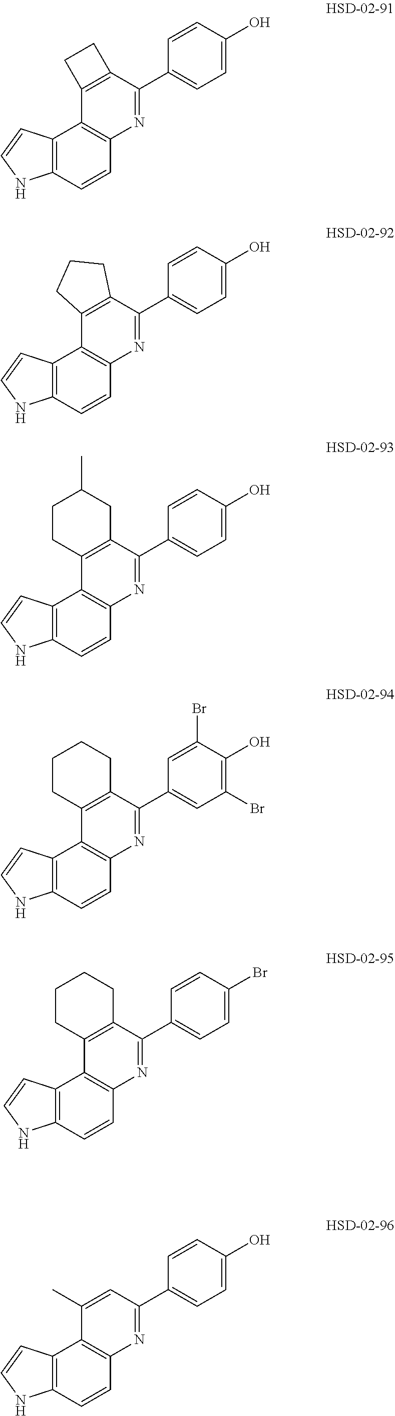

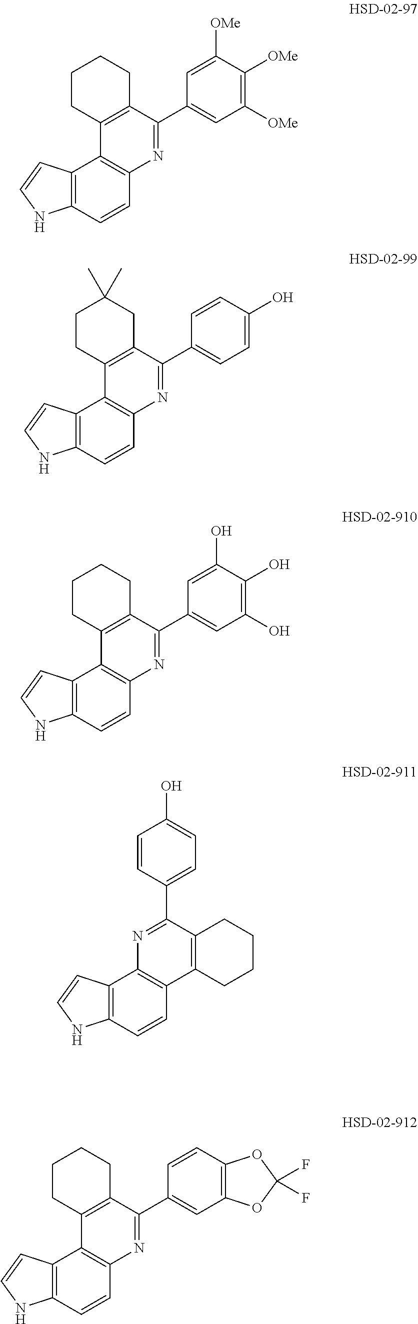 [SMILES: COc1cc(-c2nc3ccc4[nH]ccc4c3c3c2CCCC3)cc(OC)c1OC ; CC1(C)CCc2c(c(-c3ccc(O)cc3)nc3ccc4[nH]ccc4c23)C1 ; Oc1cc(-c2nc3ccc4[nH]ccc4c3c3c2CCCC3)cc(O)c1O ; Oc1ccc(-c2nc3c(ccc4[nH]ccc43)c3c2CCCC3)cc1 ; FC1(F)Oc2ccc(-c3nc4ccc5[nH]ccc5c4c4c3CCCC4)cc2O1]

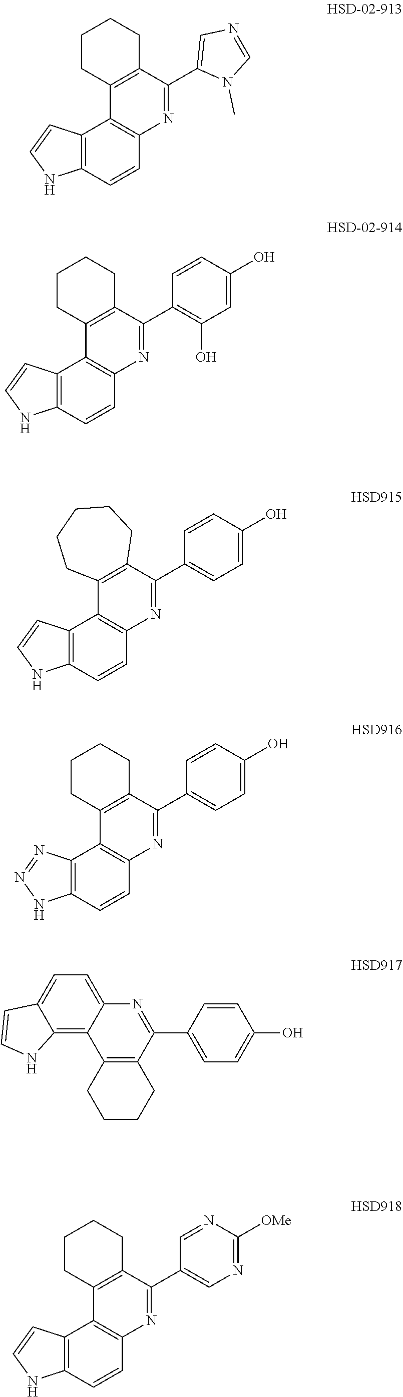 [SMILES: Cn1cncc1-c1nc2ccc3[nH]ccc3c2c2c1CCCC2 ; Oc1ccc(-c2nc3ccc4[nH]ccc4c3c3c2CCCC3)c(O)c1 ; Oc1ccc(-c2nc3ccc4[nH]ccc4c3c3c2CCCCC3)cc1 ; Oc1ccc(-c2nc3ccc4[nH]nnc4c3c3c2CCCC3)cc1 ; Oc1ccc(-c2nc3ccc4cc[nH]c4c3c3c2CCCC3)cc1 ; COc1ncc(-c2nc3ccc4[nH]ccc4c3c3c2CCCC3)cn1]

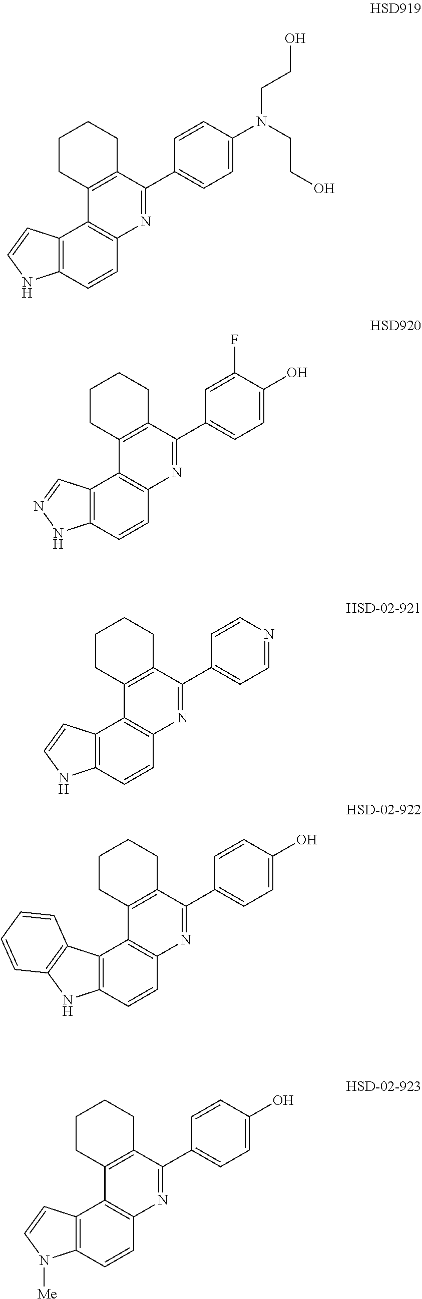 [SMILES: OCCN(CCO)c1ccc(-c2nc3ccc4[nH]ccc4c3c3c2CCCC3)cc1 ; Oc1ccc(-c2nc3ccc4[nH]ncc4c3c3c2CCCC3)cc1F ; c1cc(-c2nc3ccc4[nH]ccc4c3c3c2CCCC3)ccn1 ; Oc1ccc(-c2nc3ccc4[nH]c5ccccc5c4c3c3c2CCCC3)cc1 ; Cn1ccc2c3c4c(c(-c5ccc(O)cc5)nc3ccc21)CCCC4]

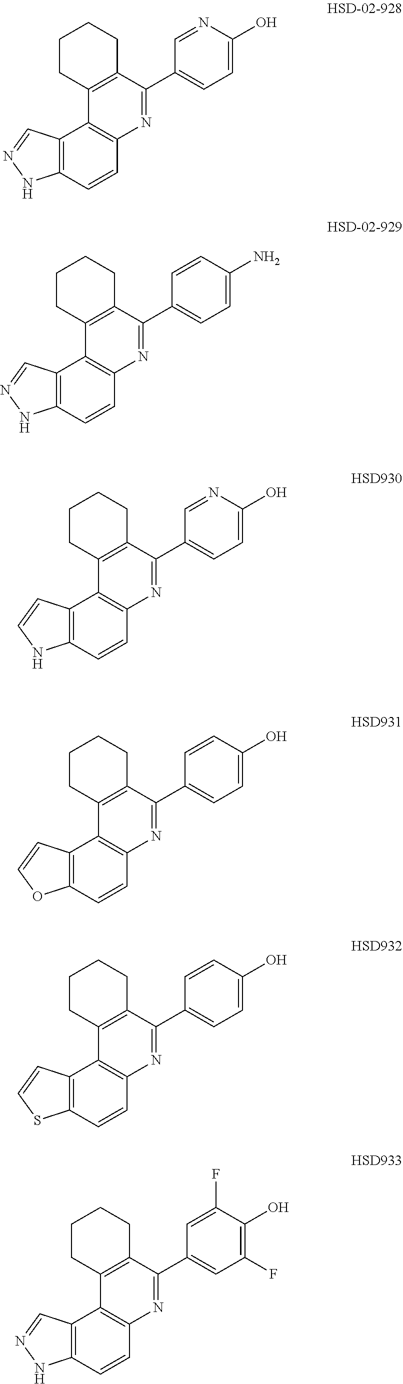 [SMILES: Oc1ccc(-c2nc3ccc4[nH]ncc4c3c3c2CCCC3)cn1 ; Nc1ccc(-c2nc3ccc4[nH]ncc4c3c3c2CCCC3)cc1 ; Oc1ccc(-c2nc3ccc4[nH]ccc4c3c3c2CCCC3)cn1 ; Oc1ccc(-c2nc3ccc4occc4c3c3c2CCCC3)cc1 ; Oc1ccc(-c2nc3ccc4sccc4c3c3c2CCCC3)cc1 ; Oc1c(F)cc(-c2nc3ccc4[nH]ncc4c3c3c2CCCC3)cc1F]

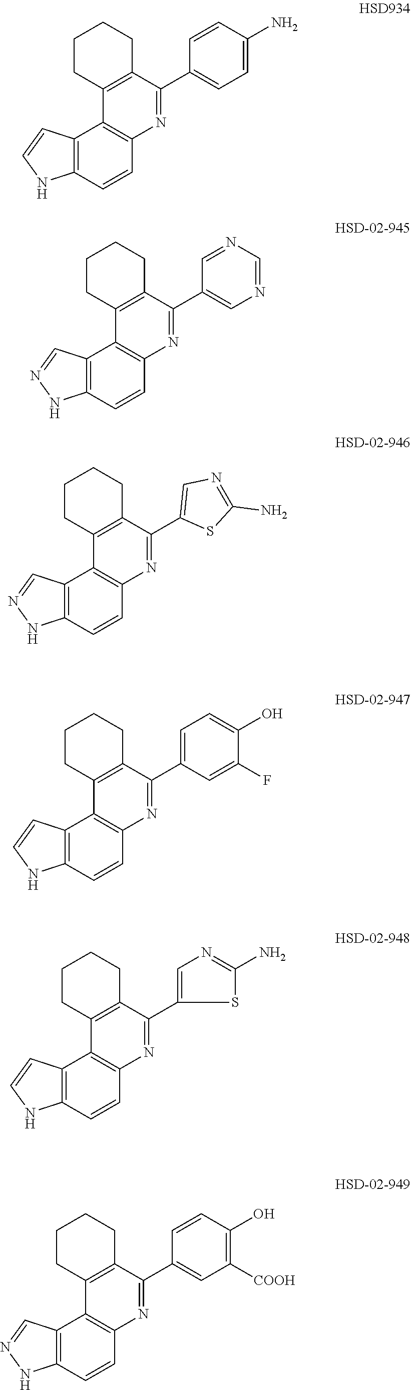 [SMILES: Nc1ccc(-c2nc3ccc4[nH]ccc4c3c3c2CCCC3)cc1 ; c1ncc(-c2nc3ccc4[nH]ncc4c3c3c2CCCC3)cn1 ; Nc1ncc(-c2nc3ccc4[nH]ncc4c3c3c2CCCC3)s1 ; Oc1ccc(-c2nc3ccc4[nH]ccc4c3c3c2CCCC3)cc1F ; Nc1ncc(-c2nc3ccc4[nH]ccc4c3c3c2CCCC3)s1 ; O=C(O)c1cc(-c2nc3ccc4[nH]ncc4c3c3c2CCCC3)ccc1O]

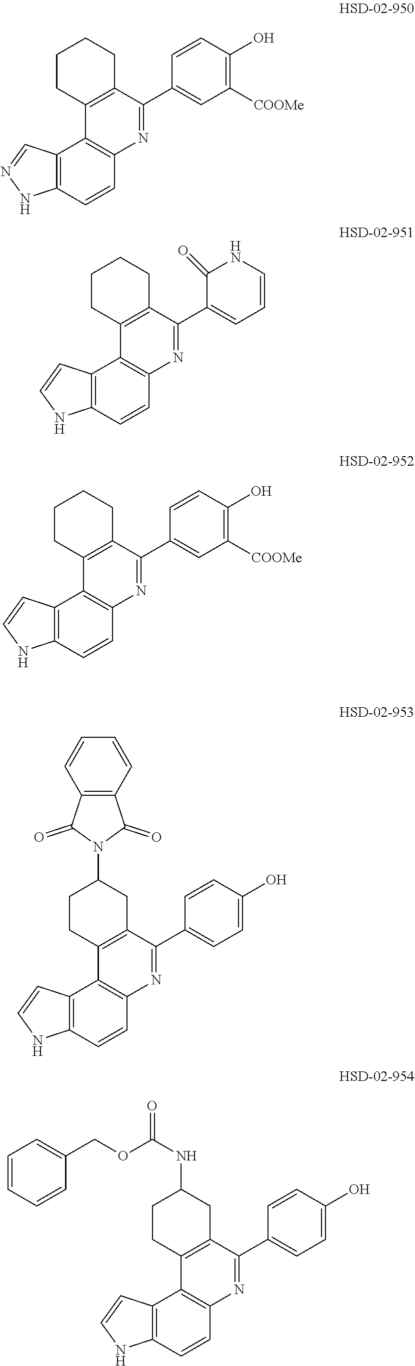 [SMILES: COC(=O)c1cc(-c2nc3ccc4[nH]ncc4c3c3c2CCCC3)ccc1O ; O=c1[nH]cccc1-c1nc2ccc3[nH]ccc3c2c2c1CCCC2 ; COC(=O)c1cc(-c2nc3ccc4[nH]ccc4c3c3c2CCCC3)ccc1O ; O=C1c2ccccc2C(=O)N1C1CCc2c(c(-c3ccc(O)cc3)nc3ccc4[nH]ccc4c23)C1 ; O=C(NC1CCc2c(c(-c3ccc(O)cc3)nc3ccc4[nH]ccc4c23)C1)OCc1ccccc1]

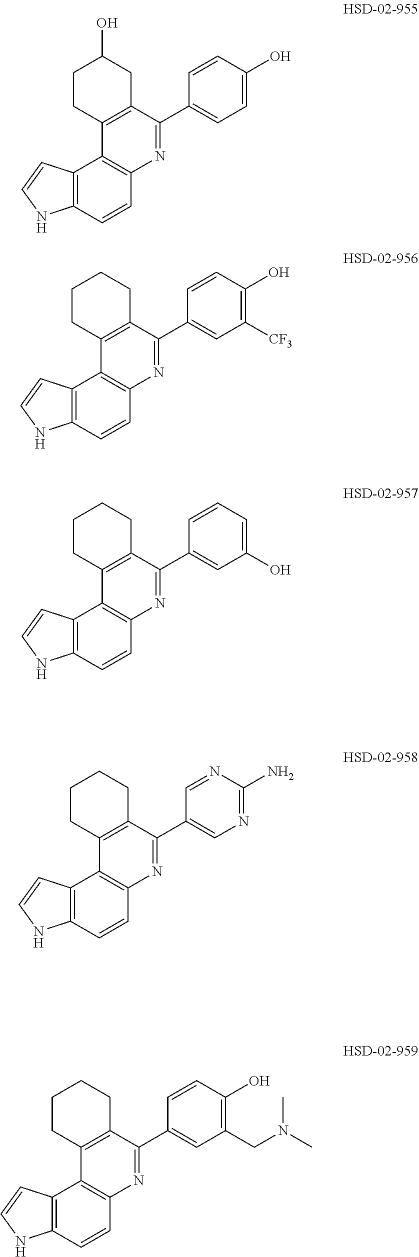 [SMILES: Oc1ccc(-c2nc3ccc4[nH]ccc4c3c3c2CC(O)CC3)cc1 ; Oc1ccc(-c2nc3ccc4[nH]ccc4c3c3c2CCCC3)cc1C(F)(F)F ; Oc1cccc(-c2nc3ccc4[nH]ccc4c3c3c2CCCC3)c1 ; Nc1ncc(-c2nc3ccc4[nH]ccc4c3c3c2CCCC3)cn1 ; CN(C)Cc1cc(-c2nc3ccc4[nH]ccc4c3c3c2CCCC3)ccc1O]

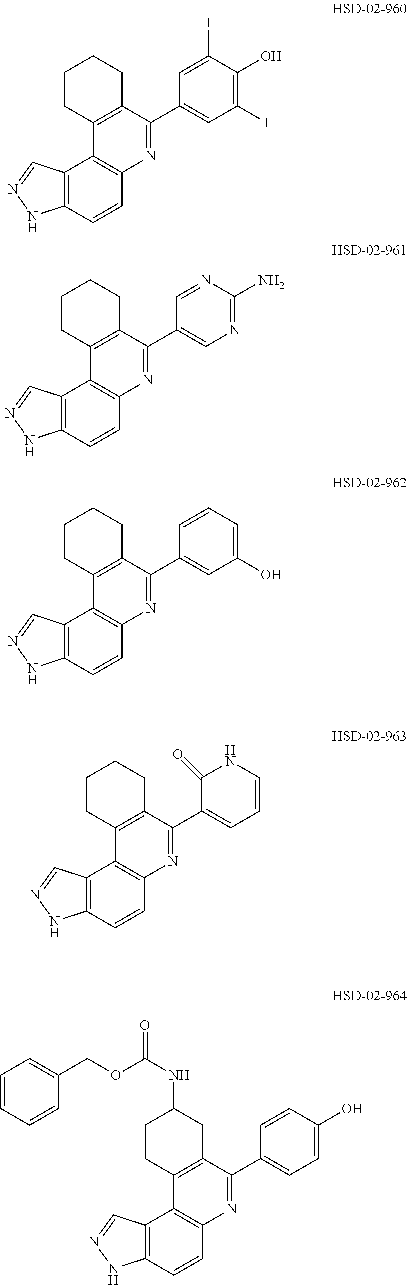 [SMILES: Oc1c(I)cc(-c2nc3ccc4[nH]ncc4c3c3c2CCCC3)cc1I ; Nc1ncc(-c2nc3ccc4[nH]ncc4c3c3c2CCCC3)cn1 ; Oc1cccc(-c2nc3ccc4[nH]ncc4c3c3c2CCCC3)c1 ; O=c1[nH]cccc1-c1nc2ccc3[nH]ncc3c2c2c1CCCC2 ; O=C(NC1CCc2c(c(-c3ccc(O)cc3)nc3ccc4[nH]ncc4c23)C1)OCc1ccccc1]

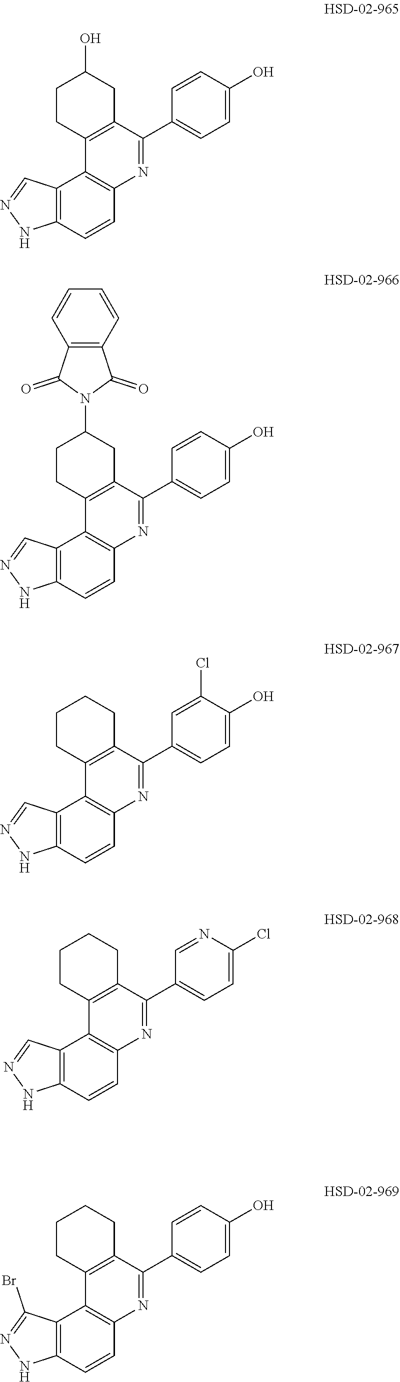 [SMILES: Oc1ccc(-c2nc3ccc4[nH]ncc4c3c3c2CC(O)CC3)cc1 ; O=C1c2ccccc2C(=O)N1C1CCc2c(c(-c3ccc(O)cc3)nc3ccc4[nH]ncc4c23)C1 ; Oc1ccc(-c2nc3ccc4[nH]ncc4c3c3c2CCCC3)cc1Cl ; Clc1ccc(-c2nc3ccc4[nH]ncc4c3c3c2CCCC3)cn1 ; Oc1ccc(-c2nc3ccc4[nH]nc(Br)c4c3c3c2CCCC3)cc1]

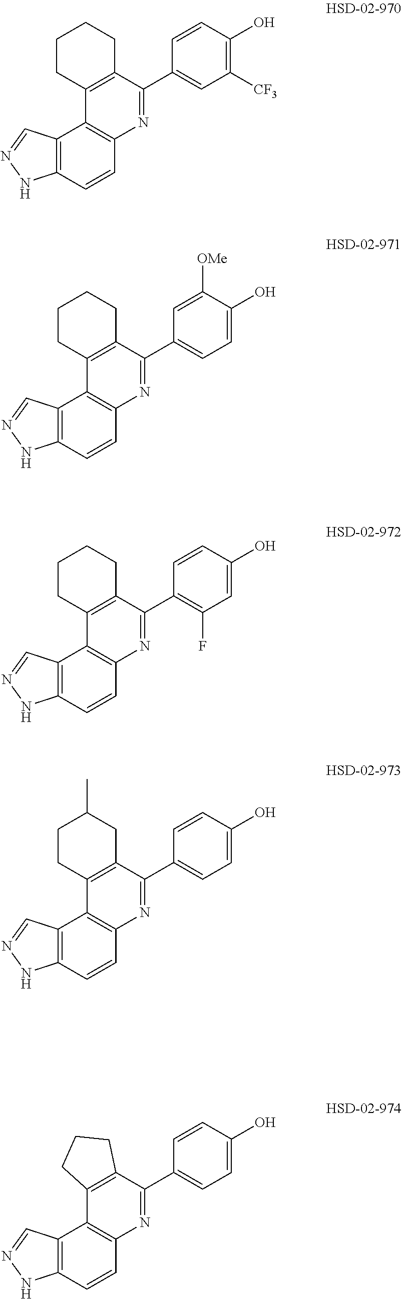 [SMILES: Oc1ccc(-c2nc3ccc4[nH]ncc4c3c3c2CCCC3)cc1C(F)(F)F ; COc1cc(-c2nc3ccc4[nH]ncc4c3c3c2CCCC3)ccc1O ; Oc1ccc(-c2nc3ccc4[nH]ncc4c3c3c2CCCC3)c(F)c1 ; CC1CCc2c(c(-c3ccc(O)cc3)nc3ccc4[nH]ncc4c23)C1 ; Oc1ccc(-c2nc3ccc4[nH]ncc4c3c3c2CCC3)cc1]

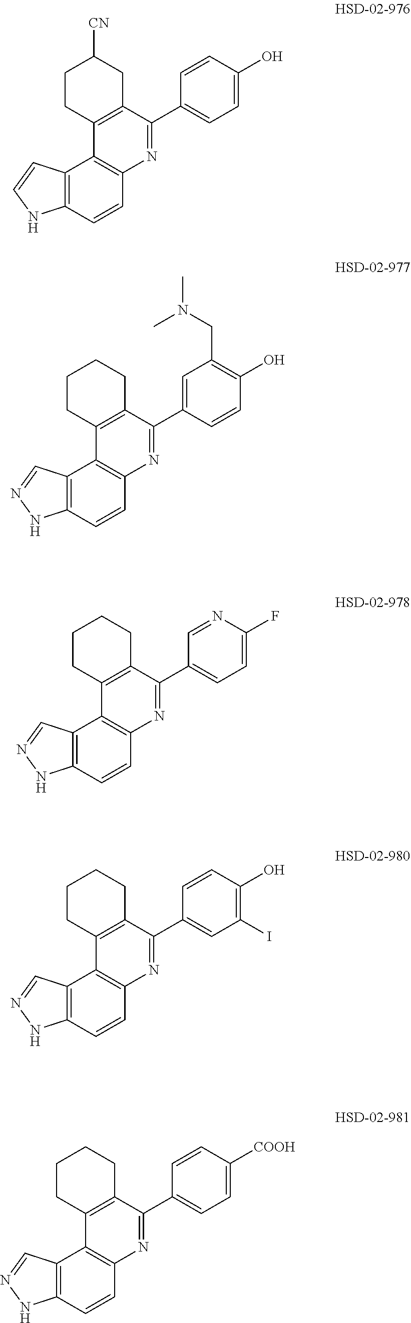 [SMILES: N#CC1CCc2c(c(-c3ccc(O)cc3)nc3ccc4[nH]ccc4c23)C1 ; CN(C)Cc1cc(-c2nc3ccc4[nH]ncc4c3c3c2CCCC3)ccc1O ; Fc1ccc(-c2nc3ccc4[nH]ncc4c3c3c2CCCC3)cn1 ; Oc1ccc(-c2nc3ccc4[nH]ncc4c3c3c2CCCC3)cc1I ; O=C(O)c1ccc(-c2nc3ccc4[nH]ncc4c3c3c2CCCC3)cc1]

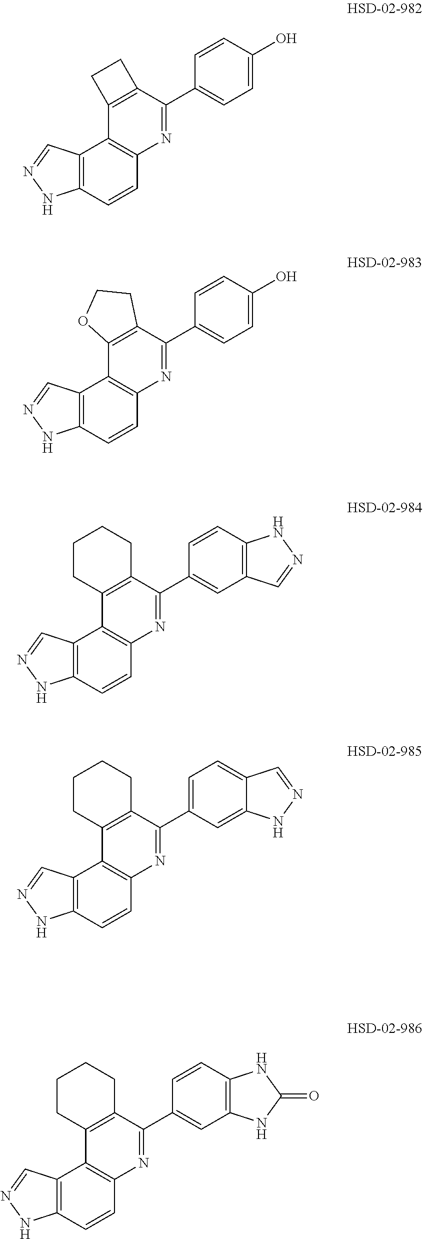 [SMILES: Oc1ccc(-c2nc3ccc4[nH]ncc4c3c3c2CC3)cc1 ; Oc1ccc(-c2nc3ccc4[nH]ncc4c3c3c2CCO3)cc1 ; c1cc2[nH]ncc2cc1-c1nc2ccc3[nH]ncc3c2c2c1CCCC2 ; c1cc2cn[nH]c2cc1-c1nc2ccc3[nH]ncc3c2c2c1CCCC2 ; O=c1[nH]c2ccc(-c3nc4ccc5[nH]ncc5c4c4c3CCCC4)cc2[nH]1]

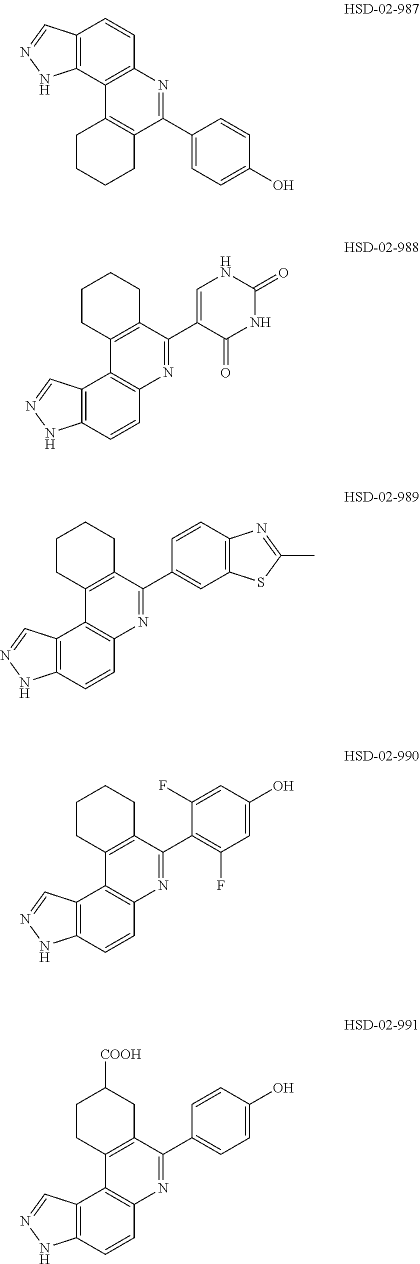 [SMILES: Oc1ccc(-c2nc3ccc4cn[nH]c4c3c3c2CCCC3)cc1 ; O=c1[nH]cc(-c2nc3ccc4[nH]ncc4c3c3c2CCCC3)c(=O)[nH]1 ; Cc1nc2ccc(-c3nc4ccc5[nH]ncc5c4c4c3CCCC4)cc2s1 ; Oc1cc(F)c(-c2nc3ccc4[nH]ncc4c3c3c2CCCC3)c(F)c1 ; O=C(O)C1CCc2c(c(-c3ccc(O)cc3)nc3ccc4[nH]ncc4c23)C1]

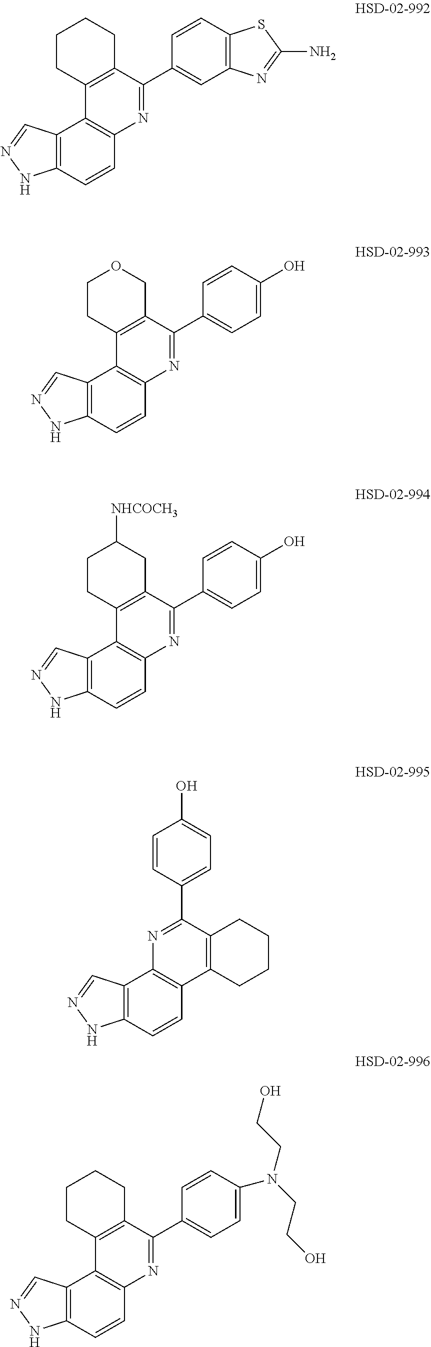

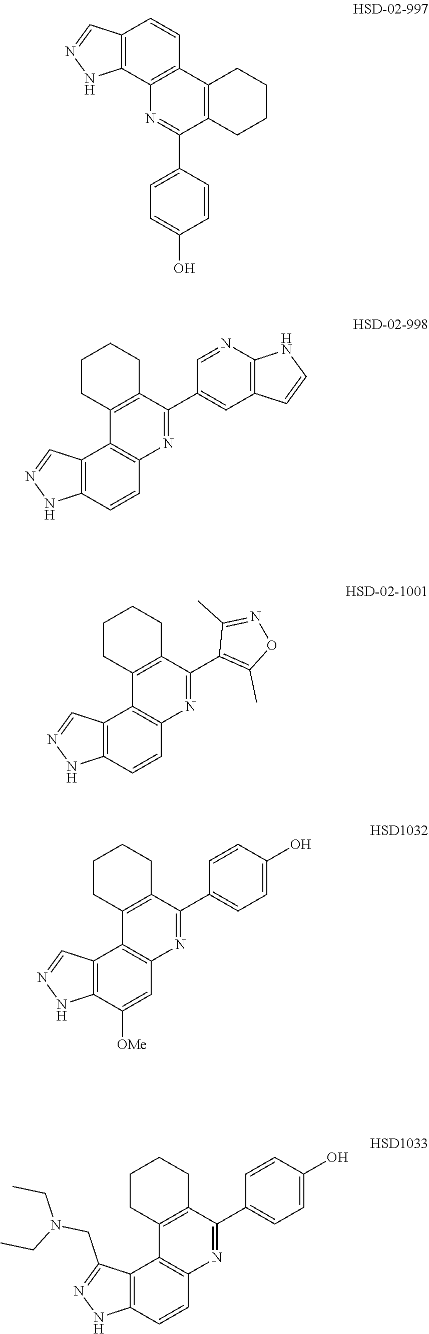 [SMILES: Oc1ccc(-c2nc3c(ccc4cn[nH]c43)c3c2CCCC3)cc1 ; c1cc2cc(-c3nc4ccc5[nH]ncc5c4c4c3CCCC4)cnc2[nH]1 ; Cc1noc(C)c1-c1nc2ccc3[nH]ncc3c2c2c1CCCC2 ; COc1cc2nc(-c3ccc(O)cc3)c3c(c2c2cn[nH]c12)CCCC3 ; CCN(CC)Cc1n[nH]c2ccc3nc(-c4ccc(O)cc4)c4c(c3c12)CCCC4]

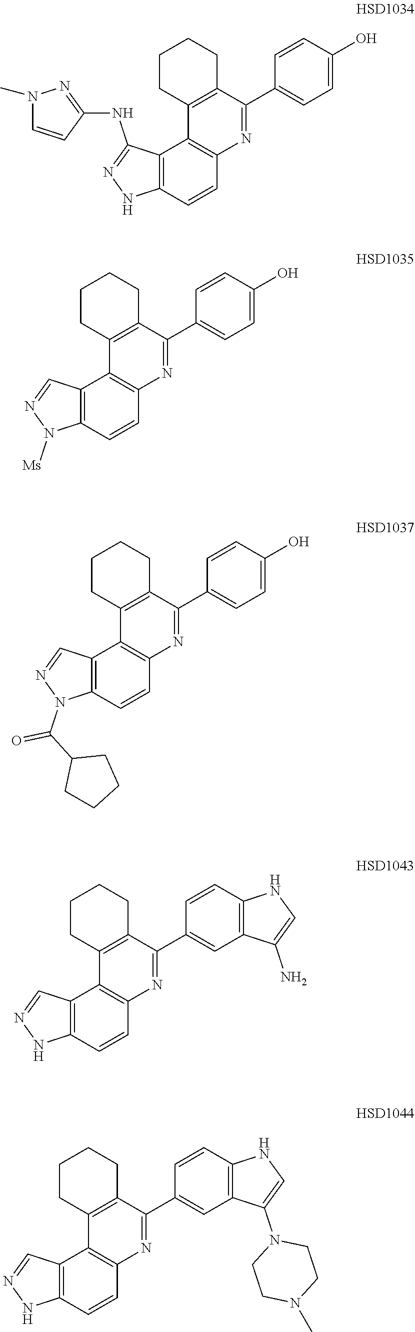 [SMILES: Cn1ccc(Nc2n[nH]c3ccc4nc(-c5ccc(O)cc5)c5c(c4c23)CCCC5)n1 ; CS(=O)(=O)n1ncc2c3c4c(c(-c5ccc(O)cc5)nc3ccc21)CCCC4 ; O=C(C1CCCC1)n1ncc2c3c4c(c(-c5ccc(O)cc5)nc3ccc21)CCCC4 ; Nc1c[nH]c2ccc(-c3nc4ccc5[nH]ncc5c4c4c3CCCC4)cc12 ; CN1CCN(c2c[nH]c3ccc(-c4nc5ccc6[nH]ncc6c5c5c4CCCC5)cc23)CC1]

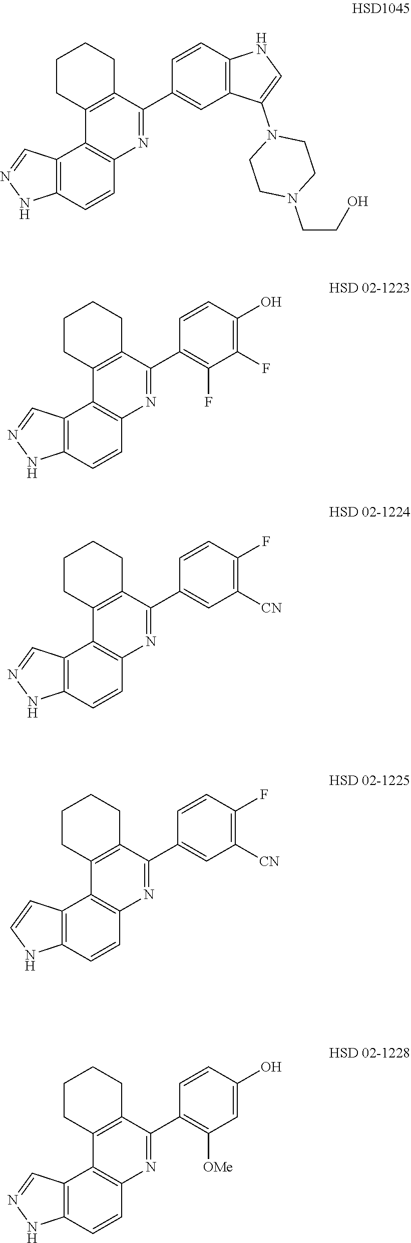 [SMILES: OCCN1CCN(c2c[nH]c3ccc(-c4nc5ccc6[nH]ncc6c5c5c4CCCC5)cc23)CC1 ; Oc1ccc(-c2nc3ccc4[nH]ncc4c3c3c2CCCC3)c(F)c1F ; N#Cc1cc(-c2nc3ccc4[nH]ncc4c3c3c2CCCC3)ccc1F ; N#Cc1cc(-c2nc3ccc4[nH]ccc4c3c3c2CCCC3)ccc1F ; COc1cc(O)ccc1-c1nc2ccc3[nH]ncc3c2c2c1CCCC2]

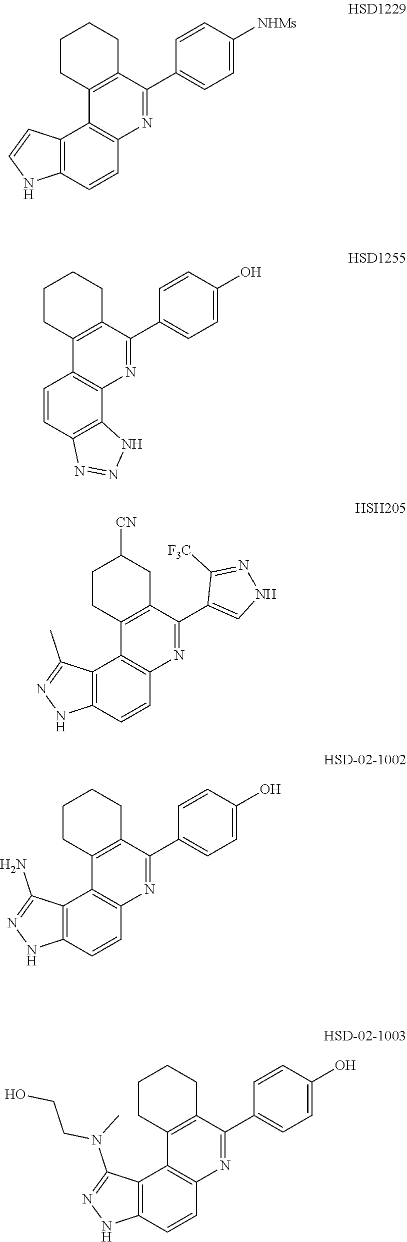

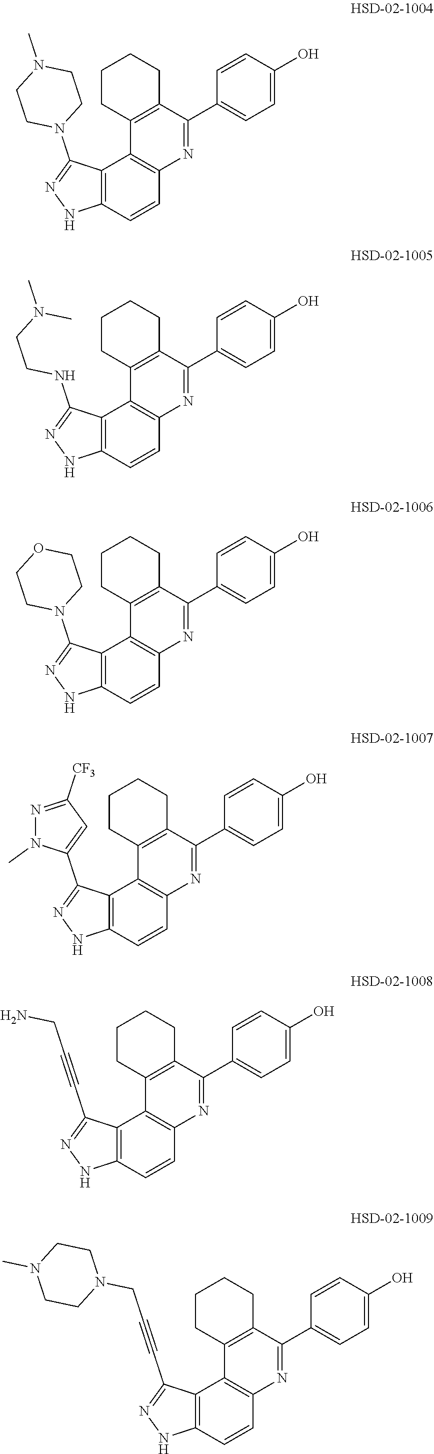 [SMILES: CN1CCN(c2n[nH]c3ccc4nc(-c5ccc(O)cc5)c5c(c4c23)CCCC5)CC1 ; CN(C)CCNc1n[nH]c2ccc3nc(-c4ccc(O)cc4)c4c(c3c12)CCCC4 ; Oc1ccc(-c2nc3ccc4[nH]nc(N5CCOCC5)c4c3c3c2CCCC3)cc1 ; Cn1nc(C(F)(F)F)cc1-c1n[nH]c2ccc3nc(-c4ccc(O)cc4)c4c(c3c12)CCCC4 ; NCC#Cc1n[nH]c2ccc3nc(-c4ccc(O)cc4)c4c(c3c12)CCCC4 ; CN1CCN(CC#Cc2n[nH]c3ccc4nc(-c5ccc(O)cc5)c5c(c4c23)CCCC5)CC1]

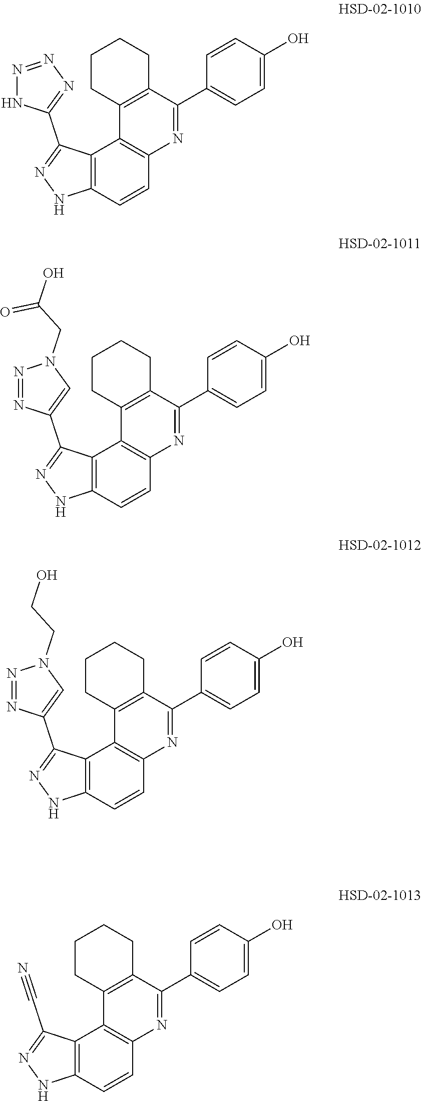 [SMILES: Oc1ccc(-c2nc3ccc4[nH]nc(-c5nnn[nH]5)c4c3c3c2CCCC3)cc1 ; O=C(O)Cn1cc(-c2n[nH]c3ccc4nc(-c5ccc(O)cc5)c5c(c4c23)CCCC5)nn1 ; OCCn1cc(-c2n[nH]c3ccc4nc(-c5ccc(O)cc5)c5c(c4c23)CCCC5)nn1 ; N#Cc1n[nH]c2ccc3nc(-c4ccc(O)cc4)c4c(c3c12)CCCC4]

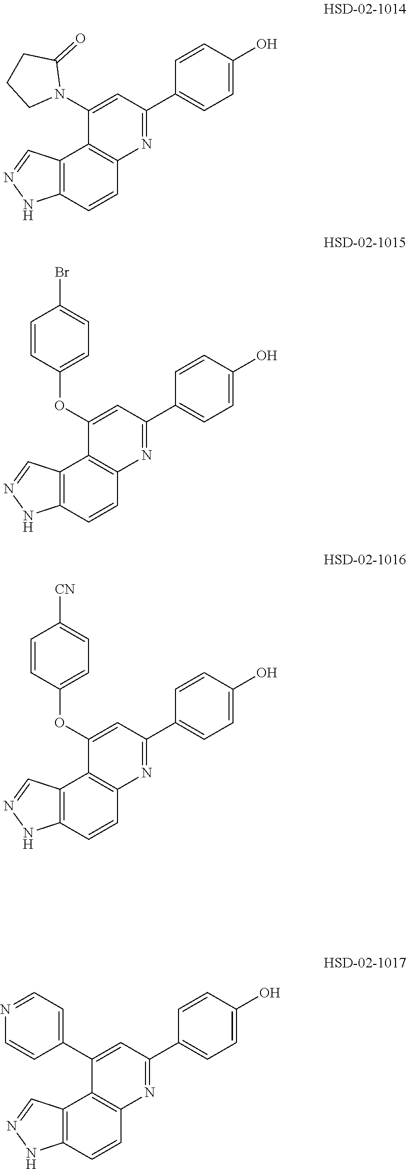 [SMILES: O=C1CCCN1c1cc(-c2ccc(O)cc2)nc2ccc3[nH]ncc3c12 ; Oc1ccc(-c2cc(Oc3ccc(Br)cc3)c3c(ccc4[nH]ncc43)n2)cc1 ; N#Cc1ccc(Oc2cc(-c3ccc(O)cc3)nc3ccc4[nH]ncc4c23)cc1 ; Oc1ccc(-c2cc(-c3ccncc3)c3c(ccc4[nH]ncc43)n2)cc1]

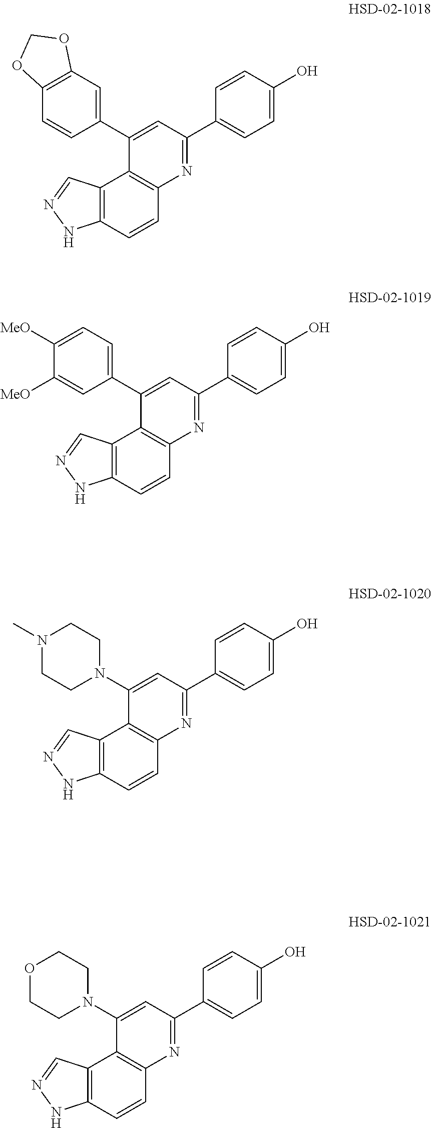 [SMILES: Oc1ccc(-c2cc(-c3ccc4c(c3)OCO4)c3c(ccc4[nH]ncc43)n2)cc1 ; COc1ccc(-c2cc(-c3ccc(O)cc3)nc3ccc4[nH]ncc4c23)cc1OC ; CN1CCN(c2cc(-c3ccc(O)cc3)nc3ccc4[nH]ncc4c23)CC1 ; Oc1ccc(-c2cc(N3CCOCC3)c3c(ccc4[nH]ncc43)n2)cc1]

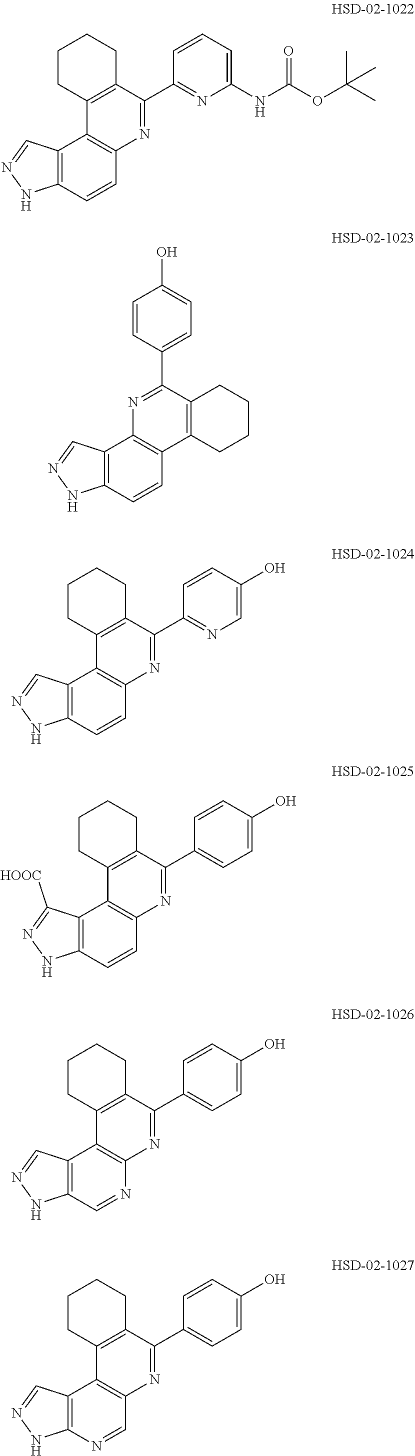 [SMILES: CC(C)(C)OC(=O)Nc1cccc(-c2nc3ccc4[nH]ncc4c3c3c2CCCC3)n1 ; Oc1ccc(-c2nc3c(ccc4[nH]ncc43)c3c2CCCC3)cc1 ; Oc1ccc(-c2nc3ccc4[nH]ncc4c3c3c2CCCC3)nc1 ; O=C(O)c1n[nH]c2ccc3nc(-c4ccc(O)cc4)c4c(c3c12)CCCC4 ; Oc1ccc(-c2nc3ncc4[nH]ncc4c3c3c2CCCC3)cc1 ; Oc1ccc(-c2nc3cnc4[nH]ncc4c3c3c2CCCC3)cc1]

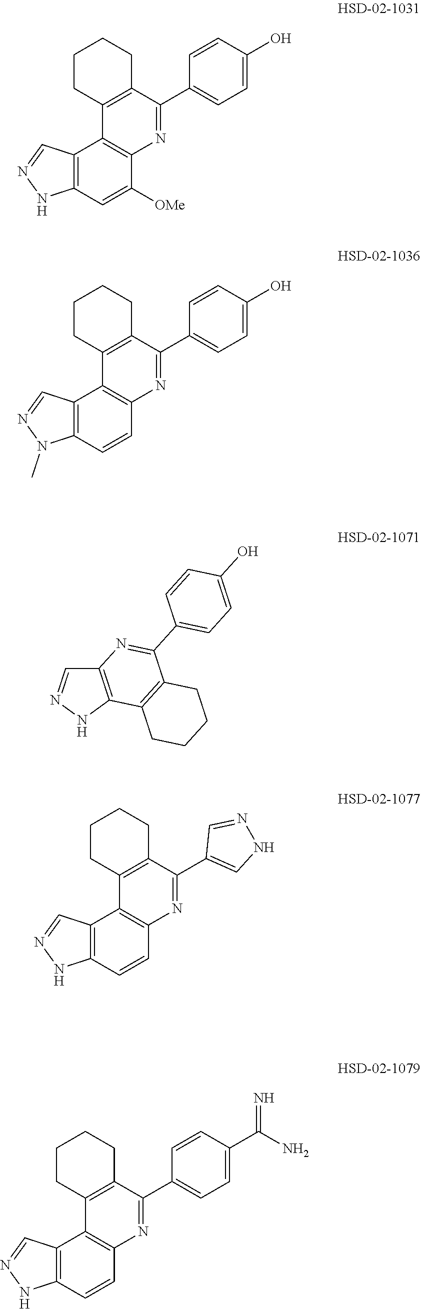 [SMILES: COc1cc2[nH]ncc2c2c3c(c(-c4ccc(O)cc4)nc12)CCCC3 ; Cn1ncc2c3c4c(c(-c5ccc(O)cc5)nc3ccc21)CCCC4 ; Oc1ccc(-c2nc3cn[nH]c3c3c2CCCC3)cc1 ; c1n[nH]cc1-c1nc2ccc3[nH]ncc3c2c2c1CCCC2 ; N=C(N)c1ccc(-c2nc3ccc4[nH]ncc4c3c3c2CCCC3)cc1]

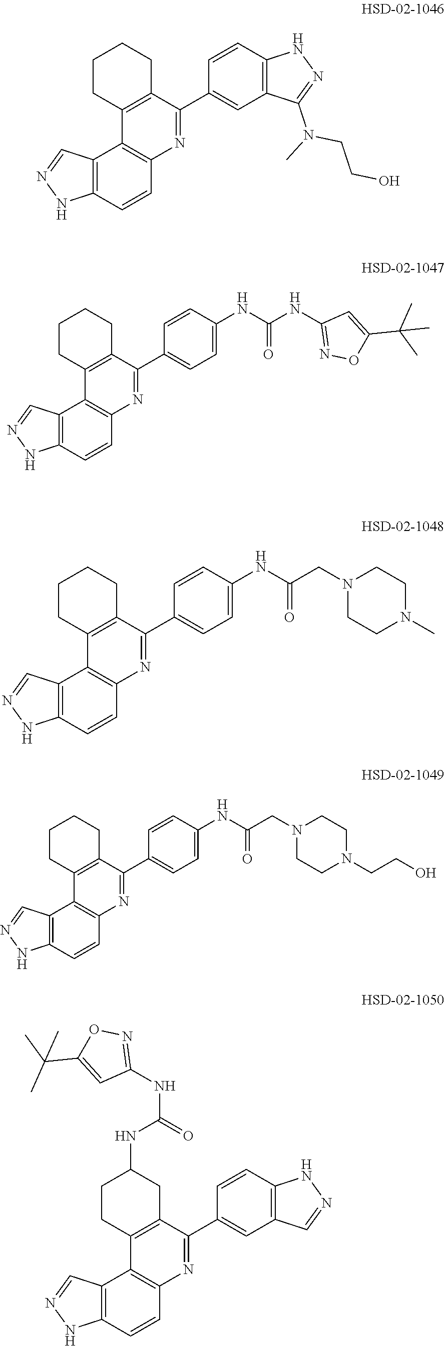

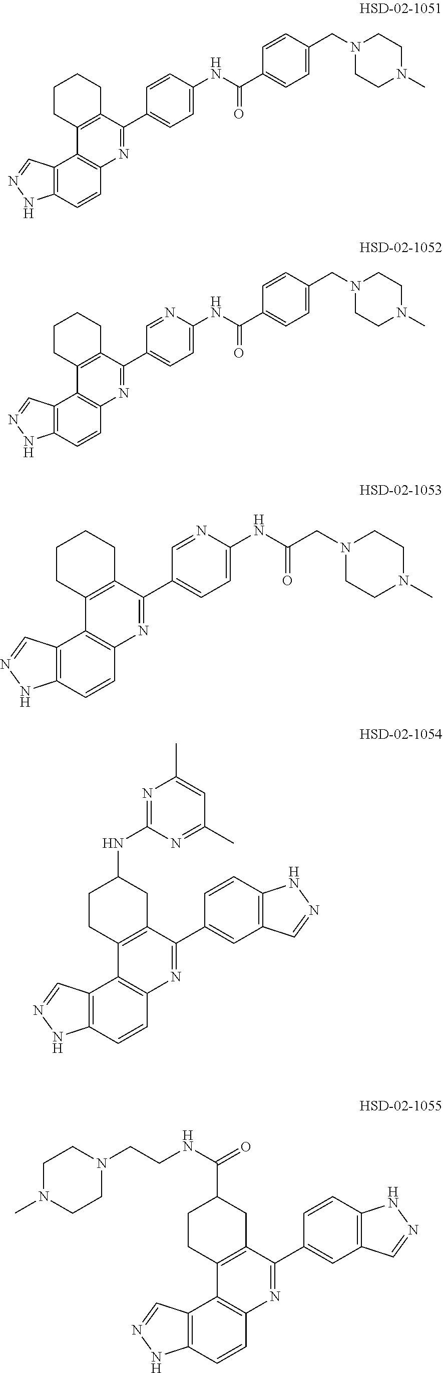

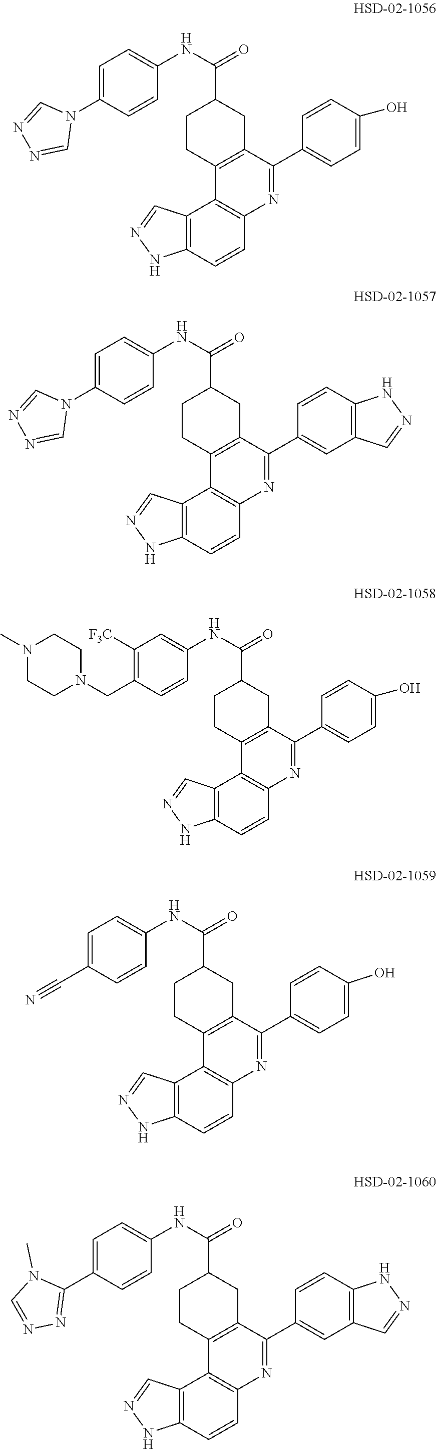 [SMILES: O=C(Nc1ccc(-n2cnnc2)cc1)C1CCc2c(c(-c3ccc(O)cc3)nc3ccc4[nH]ncc4c23)C1 ; O=C(Nc1ccc(-n2cnnc2)cc1)C1CCc2c(c(-c3ccc4[nH]ncc4c3)nc3ccc4[nH]ncc4c23)C1 ; CN1CCN(Cc2ccc(NC(=O)C3CCc4c(c(-c5ccc(O)cc5)nc5ccc6[nH]ncc6c45)C3)cc2C(F)(F)F)CC1 ; N#Cc1ccc(NC(=O)C2CCc3c(c(-c4ccc(O)cc4)nc4ccc5[nH]ncc5c34)C2)cc1 ; Cn1cnnc1-c1ccc(NC(=O)C2CCc3c(c(-c4ccc5[nH]ncc5c4)nc4ccc5[nH]ncc5c34)C2)cc1]

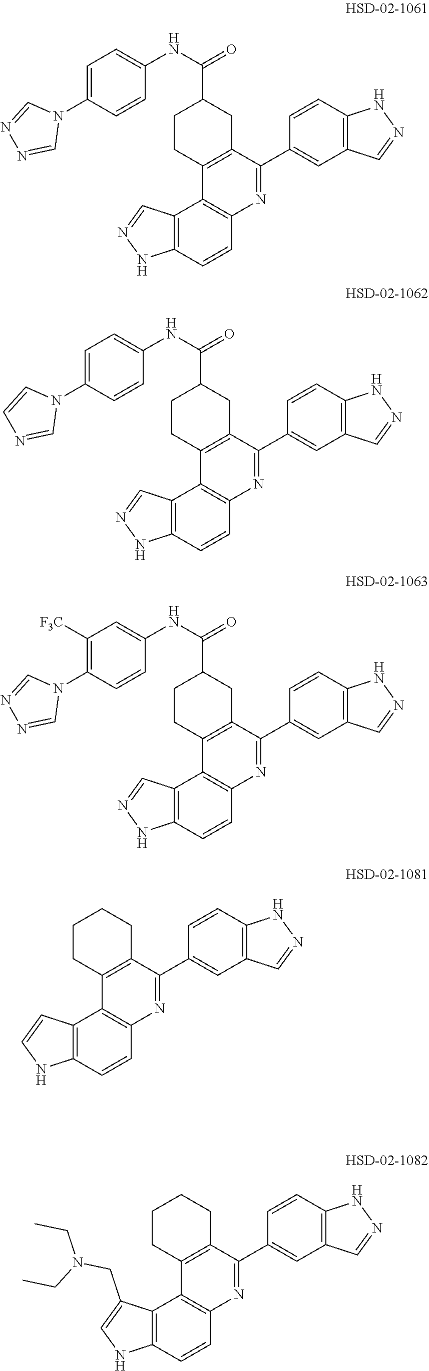 [SMILES: O=C(Nc1ccc(-n2cnnc2)cc1)C1CCc2c(c(-c3ccc4[nH]ncc4c3)nc3ccc4[nH]ncc4c23)C1 ; O=C(Nc1ccc(-n2ccnc2)cc1)C1CCc2c(c(-c3ccc4[nH]ncc4c3)nc3ccc4[nH]ncc4c23)C1 ; O=C(Nc1ccc(-n2cnnc2)c(C(F)(F)F)c1)C1CCc2c(c(-c3ccc4[nH]ncc4c3)nc3ccc4[nH]ncc4c23)C1 ; c1cc2c(ccc3nc(-c4ccc5[nH]ncc5c4)c4c(c32)CCCC4)[nH]1 ; CCN(CC)Cc1c[nH]c2ccc3nc(-c4ccc5[nH]ncc5c4)c4c(c3c12)CCCC4]

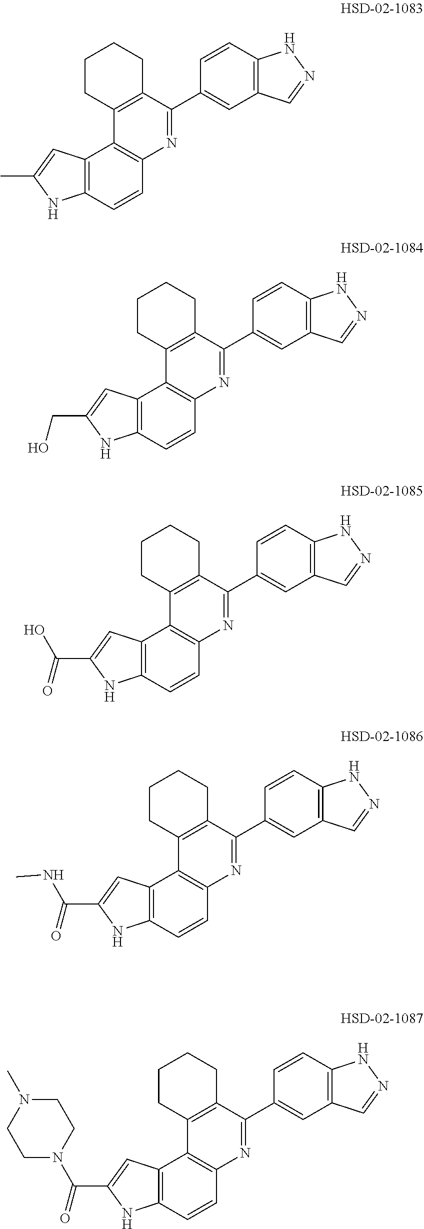 [SMILES: Cc1cc2c(ccc3nc(-c4ccc5[nH]ncc5c4)c4c(c32)CCCC4)[nH]1 ; OCc1cc2c(ccc3nc(-c4ccc5[nH]ncc5c4)c4c(c32)CCCC4)[nH]1 ; O=C(O)c1cc2c(ccc3nc(-c4ccc5[nH]ncc5c4)c4c(c32)CCCC4)[nH]1 ; CNC(=O)c1cc2c(ccc3nc(-c4ccc5[nH]ncc5c4)c4c(c32)CCCC4)[nH]1 ; CN1CCN(C(=O)c2cc3c(ccc4nc(-c5ccc6[nH]ncc6c5)c5c(c43)CCCC5)[nH]2)CC1]

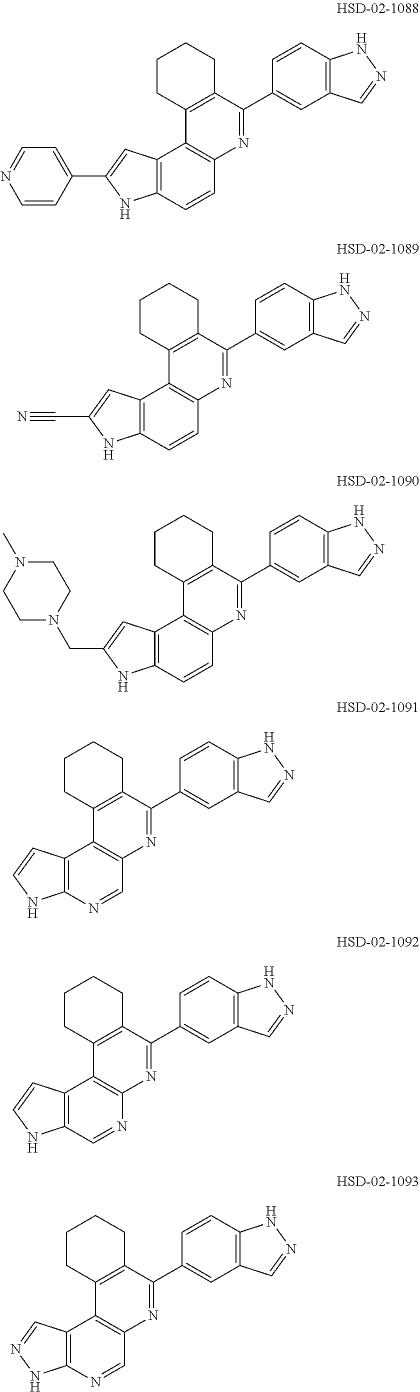 [SMILES: c1cc(-c2cc3c(ccc4nc(-c5ccc6[nH]ncc6c5)c5c(c43)CCCC5)[nH]2)ccn1 ; N#Cc1cc2c(ccc3nc(-c4ccc5[nH]ncc5c4)c4c(c32)CCCC4)[nH]1 ; CN1CCN(Cc2cc3c(ccc4nc(-c5ccc6[nH]ncc6c5)c5c(c43)CCCC5)[nH]2)CC1 ; c1cc2c(ncc3nc(-c4ccc5[nH]ncc5c4)c4c(c32)CCCC4)[nH]1 ; c1cc2c(cnc3nc(-c4ccc5[nH]ncc5c4)c4c(c32)CCCC4)[nH]1 ; c1cc2[nH]ncc2cc1-c1nc2cnc3[nH]ncc3c2c2c1CCCC2]

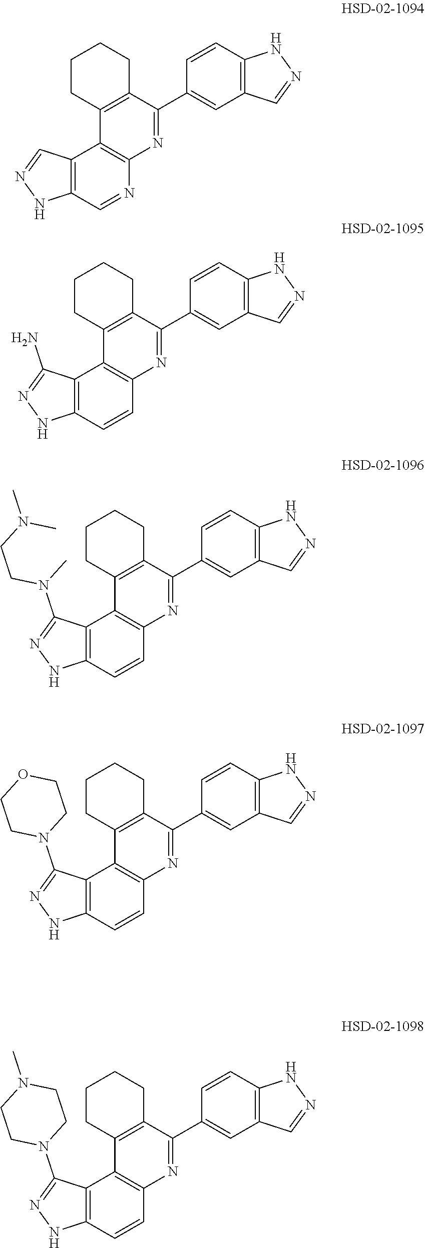 [SMILES: c1cc2[nH]ncc2cc1-c1nc2ncc3[nH]ncc3c2c2c1CCCC2 ; Nc1n[nH]c2ccc3nc(-c4ccc5[nH]ncc5c4)c4c(c3c12)CCCC4 ; CN(C)CCN(C)c1n[nH]c2ccc3nc(-c4ccc5[nH]ncc5c4)c4c(c3c12)CCCC4 ; c1cc2[nH]ncc2cc1-c1nc2ccc3[nH]nc(N4CCOCC4)c3c2c2c1CCCC2 ; CN1CCN(c2n[nH]c3ccc4nc(-c5ccc6[nH]ncc6c5)c5c(c4c23)CCCC5)CC1]

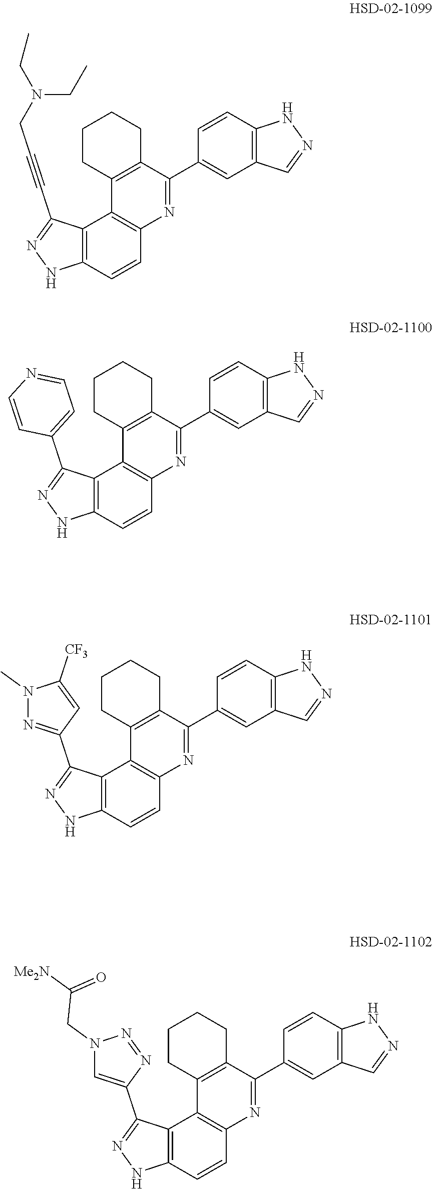 [SMILES: CCN(CC)CC#Cc1n[nH]c2ccc3nc(-c4ccc5[nH]ncc5c4)c4c(c3c12)CCCC4 ; c1cc(-c2n[nH]c3ccc4nc(-c5ccc6[nH]ncc6c5)c5c(c4c23)CCCC5)ccn1 ; Cn1nc(-c2n[nH]c3ccc4nc(-c5ccc6[nH]ncc6c5)c5c(c4c23)CCCC5)cc1C(F)(F)F ; CN(C)C(=O)Cn1cc(-c2n[nH]c3ccc4nc(-c5ccc6[nH]ncc6c5)c5c(c4c23)CCCC5)nn1]

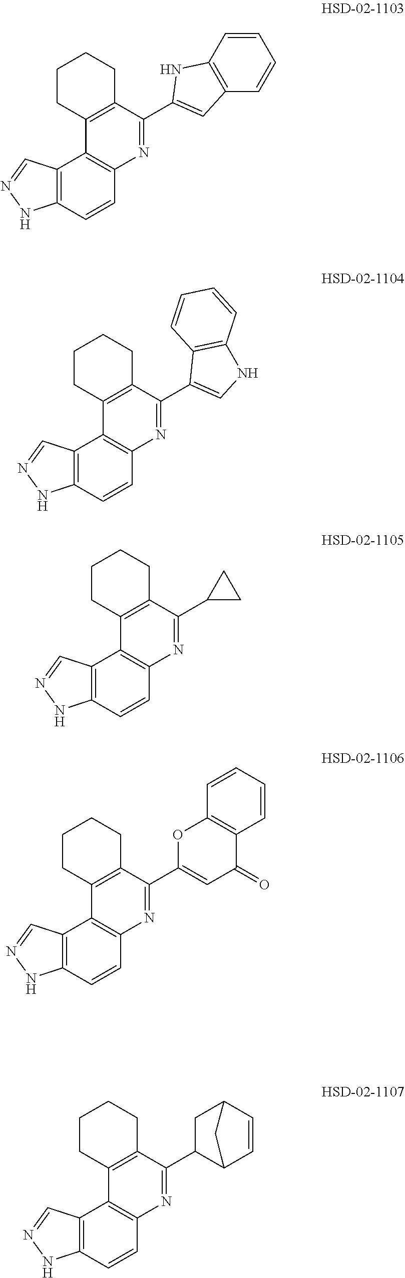 [SMILES: c1ccc2[nH]c(-c3nc4ccc5[nH]ncc5c4c4c3CCCC4)cc2c1 ; c1ccc2c(-c3nc4ccc5[nH]ncc5c4c4c3CCCC4)c[nH]c2c1 ; c1cc2[nH]ncc2c2c3c(c(C4CC4)nc12)CCCC3 ; O=c1cc(-c2nc3ccc4[nH]ncc4c3c3c2CCCC3)oc2ccccc12 ; C1=CC2CC1CC2c1nc2ccc3[nH]ncc3c2c2c1CCCC2]

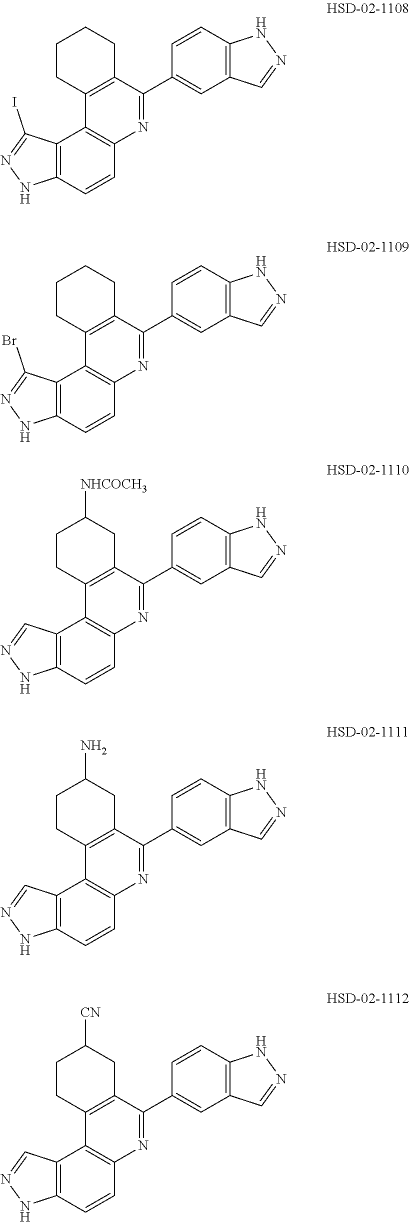

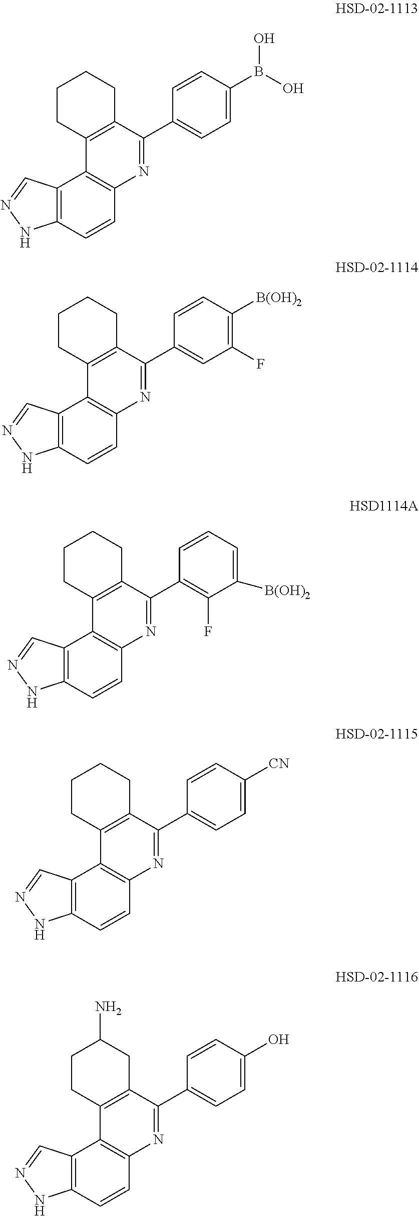 [SMILES: OB(O)c1ccc(-c2nc3ccc4[nH]ncc4c3c3c2CCCC3)cc1 ; OB(O)c1ccc(-c2nc3ccc4[nH]ncc4c3c3c2CCCC3)cc1F ; OB(O)c1cccc(-c2nc3ccc4[nH]ncc4c3c3c2CCCC3)c1F ; N#Cc1ccc(-c2nc3ccc4[nH]ncc4c3c3c2CCCC3)cc1 ; NC1CCc2c(c(-c3ccc(O)cc3)nc3ccc4[nH]ncc4c23)C1]

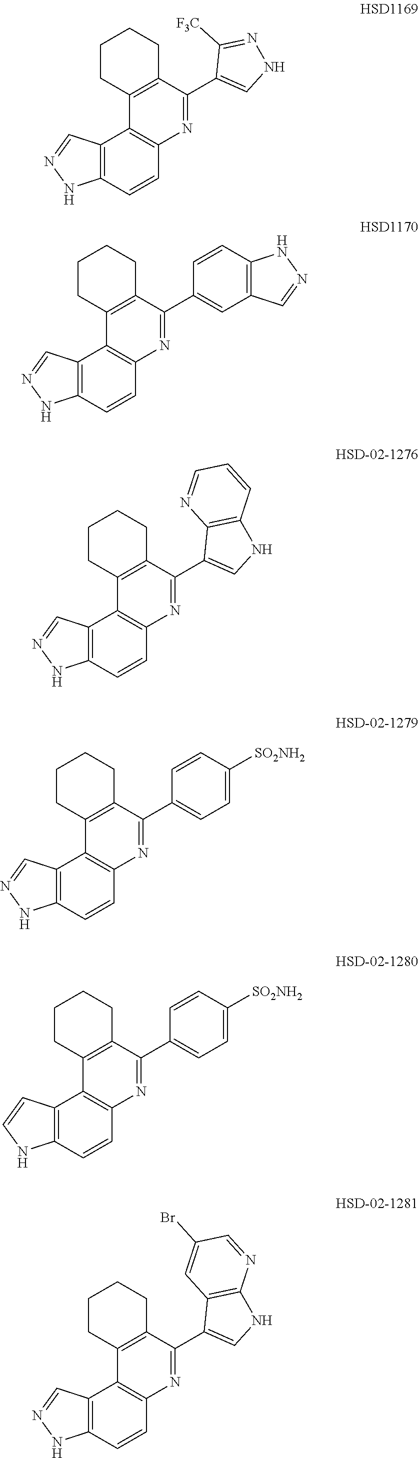 [SMILES: FC(F)(F)c1n[nH]cc1-c1nc2ccc3[nH]ncc3c2c2c1CCCC2 ; c1cc2[nH]ncc2cc1-c1nc2ccc3[nH]ncc3c2c2c1CCCC2 ; c1cnc2c(-c3nc4ccc5[nH]ncc5c4c4c3CCCC4)c[nH]c2c1 ; NS(=O)(=O)c1ccc(-c2nc3ccc4[nH]ncc4c3c3c2CCCC3)cc1 ; NS(=O)(=O)c1ccc(-c2nc3ccc4[nH]ccc4c3c3c2CCCC3)cc1 ; Brc1cnc2[nH]cc(-c3nc4ccc5[nH]ncc5c4c4c3CCCC4)c2c1]

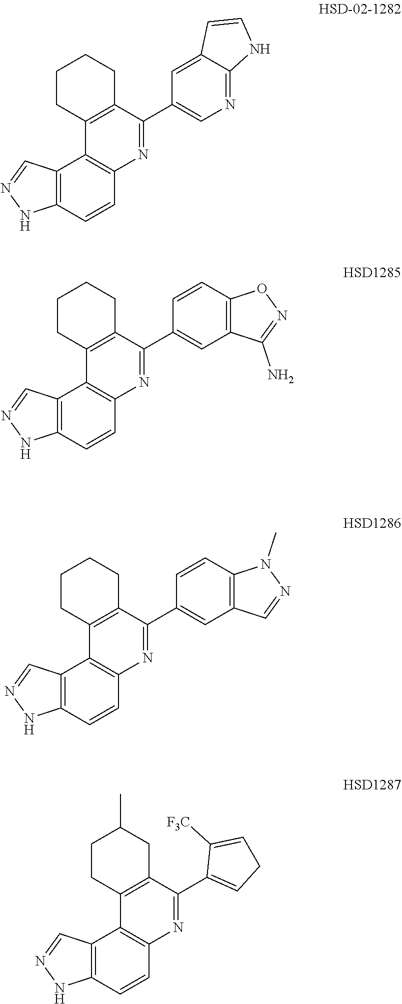 [SMILES: c1cc2cc(-c3nc4ccc5[nH]ncc5c4c4c3CCCC4)cnc2[nH]1 ; Nc1noc2ccc(-c3nc4ccc5[nH]ncc5c4c4c3CCCC4)cc12 ; Cn1ncc2cc(-c3nc4ccc5[nH]ncc5c4c4c3CCCC4)ccc21 ; CC1CCc2c(c(C3=CCC=C3C(F)(F)F)nc3ccc4[nH]ncc4c23)C1]

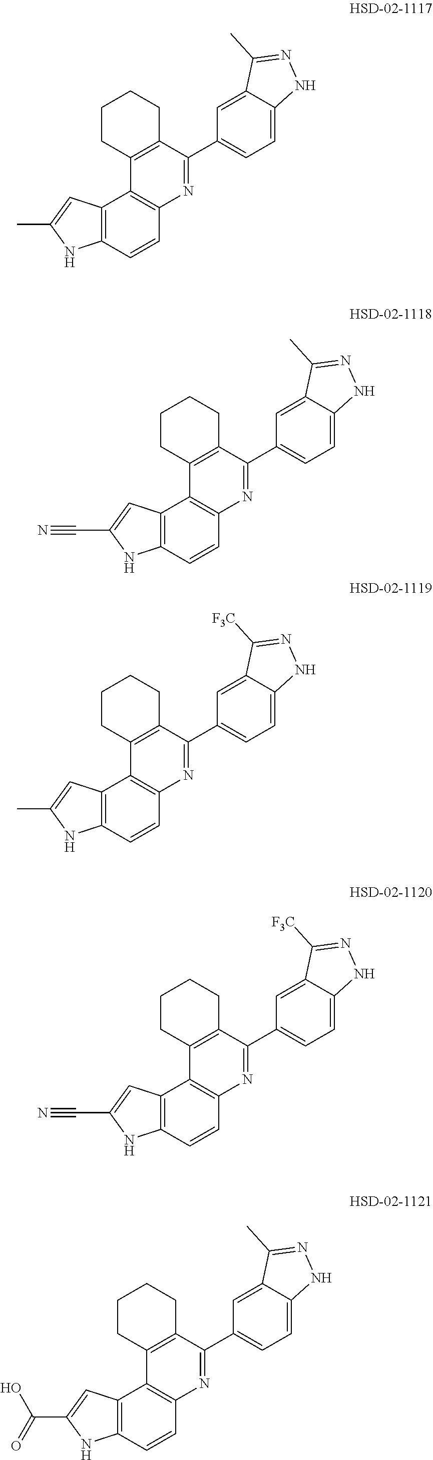 [SMILES: Cc1cc2c(ccc3nc(-c4ccc5[nH]nc(C)c5c4)c4c(c32)CCCC4)[nH]1 ; Cc1n[nH]c2ccc(-c3nc4ccc5[nH]c(C#N)cc5c4c4c3CCCC4)cc12 ; Cc1cc2c(ccc3nc(-c4ccc5[nH]nc(C(F)(F)F)c5c4)c4c(c32)CCCC4)[nH]1 ; N#Cc1cc2c(ccc3nc(-c4ccc5[nH]nc(C(F)(F)F)c5c4)c4c(c32)CCCC4)[nH]1 ; Cc1n[nH]c2ccc(-c3nc4ccc5[nH]c(C(=O)O)cc5c4c4c3CCCC4)cc12]

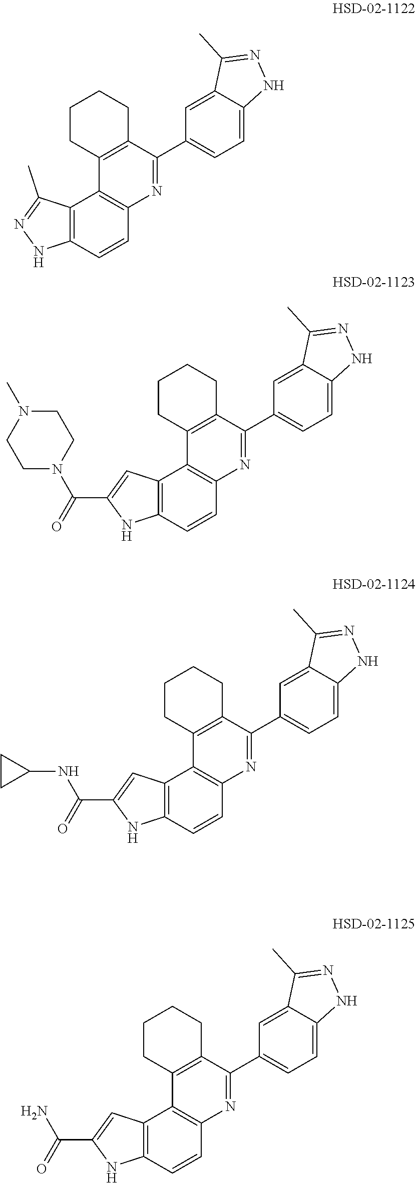 [SMILES: Cc1n[nH]c2ccc(-c3nc4ccc5[nH]nc(C)c5c4c4c3CCCC4)cc12 ; Cc1n[nH]c2ccc(-c3nc4ccc5[nH]c(C(=O)N6CCN(C)CC6)cc5c4c4c3CCCC4)cc12 ; Cc1n[nH]c2ccc(-c3nc4ccc5[nH]c(C(=O)NC6CC6)cc5c4c4c3CCCC4)cc12 ; Cc1n[nH]c2ccc(-c3nc4ccc5[nH]c(C(N)=O)cc5c4c4c3CCCC4)cc12]

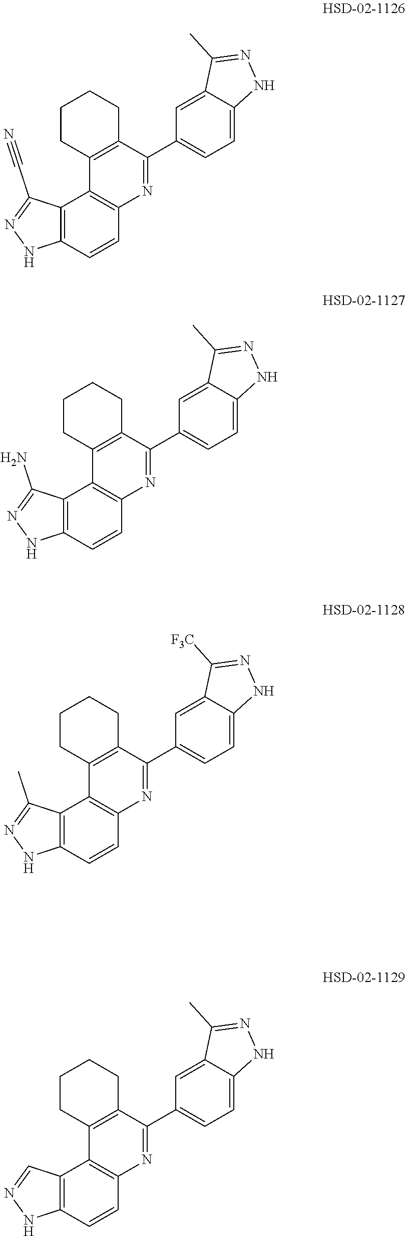 [SMILES: Cc1n[nH]c2ccc(-c3nc4ccc5[nH]nc(C#N)c5c4c4c3CCCC4)cc12 ; Cc1n[nH]c2ccc(-c3nc4ccc5[nH]nc(N)c5c4c4c3CCCC4)cc12 ; Cc1n[nH]c2ccc3nc(-c4ccc5[nH]nc(C(F)(F)F)c5c4)c4c(c3c12)CCCC4 ; Cc1n[nH]c2ccc(-c3nc4ccc5[nH]ncc5c4c4c3CCCC4)cc12]

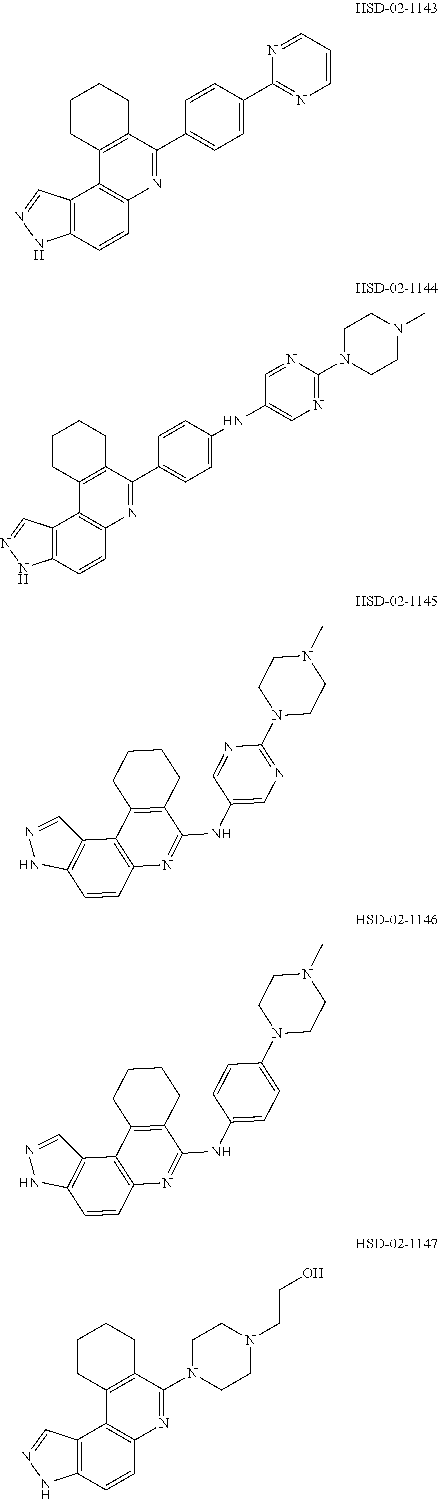 [SMILES: c1cnc(-c2ccc(-c3nc4ccc5[nH]ncc5c4c4c3CCCC4)cc2)nc1 ; CN1CCN(c2ncc(Nc3ccc(-c4nc5ccc6[nH]ncc6c5c5c4CCCC5)cc3)cn2)CC1 ; CN1CCN(c2ncc(Nc3nc4ccc5[nH]ncc5c4c4c3CCCC4)cn2)CC1 ; CN1CCN(c2ccc(Nc3nc4ccc5[nH]ncc5c4c4c3CCCC4)cc2)CC1 ; OCCN1CCN(c2nc3ccc4[nH]ncc4c3c3c2CCCC3)CC1]

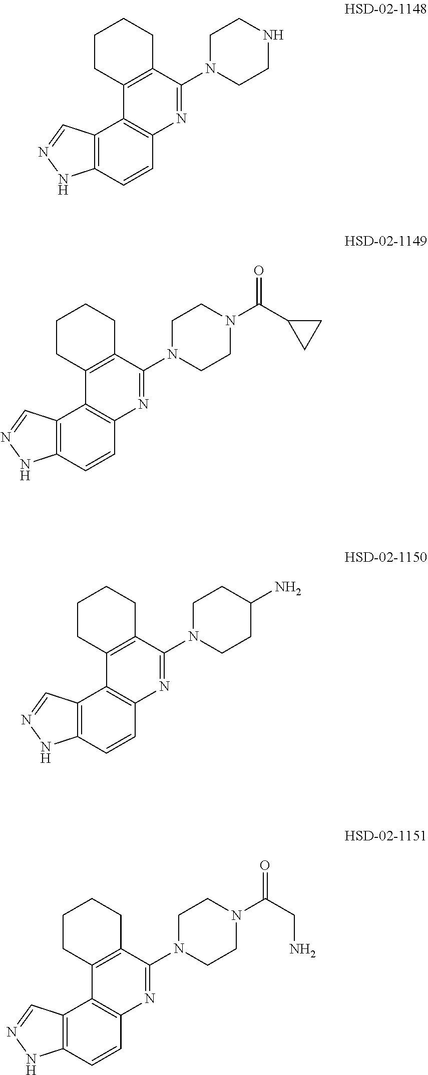 [SMILES: c1cc2[nH]ncc2c2c3c(c(N4CCNCC4)nc12)CCCC3 ; O=C(C1CC1)N1CCN(c2nc3ccc4[nH]ncc4c3c3c2CCCC3)CC1 ; NC1CCN(c2nc3ccc4[nH]ncc4c3c3c2CCCC3)CC1 ; NCC(=O)N1CCN(c2nc3ccc4[nH]ncc4c3c3c2CCCC3)CC1]

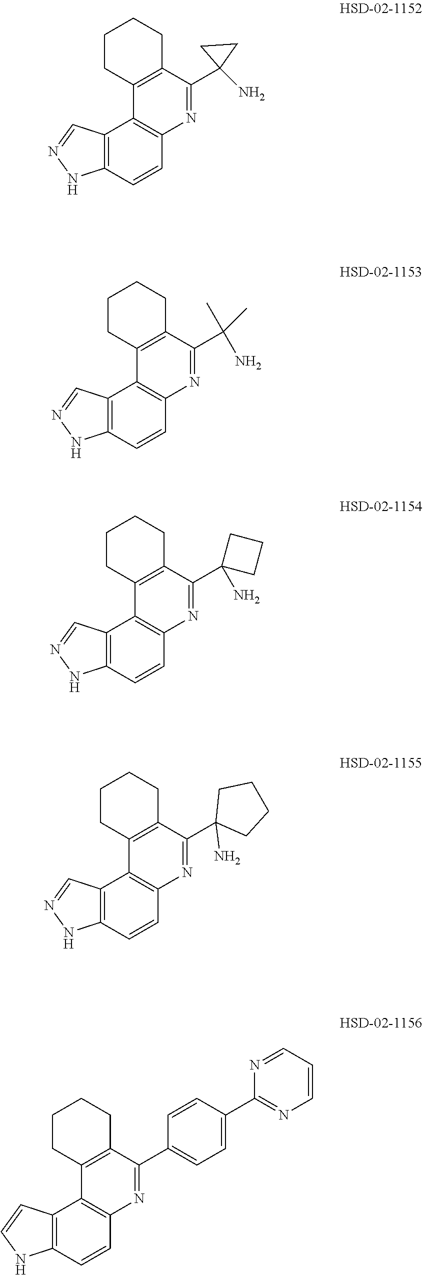 [SMILES: NC1(c2nc3ccc4[nH]ncc4c3c3c2CCCC3)CC1 ; CC(C)(N)c1nc2ccc3[nH]ncc3c2c2c1CCCC2 ; NC1(c2nc3ccc4[nH]ncc4c3c3c2CCCC3)CCC1 ; NC1(c2nc3ccc4[nH]ncc4c3c3c2CCCC3)CCCC1 ; c1cnc(-c2ccc(-c3nc4ccc5[nH]ccc5c4c4c3CCCC4)cc2)nc1]

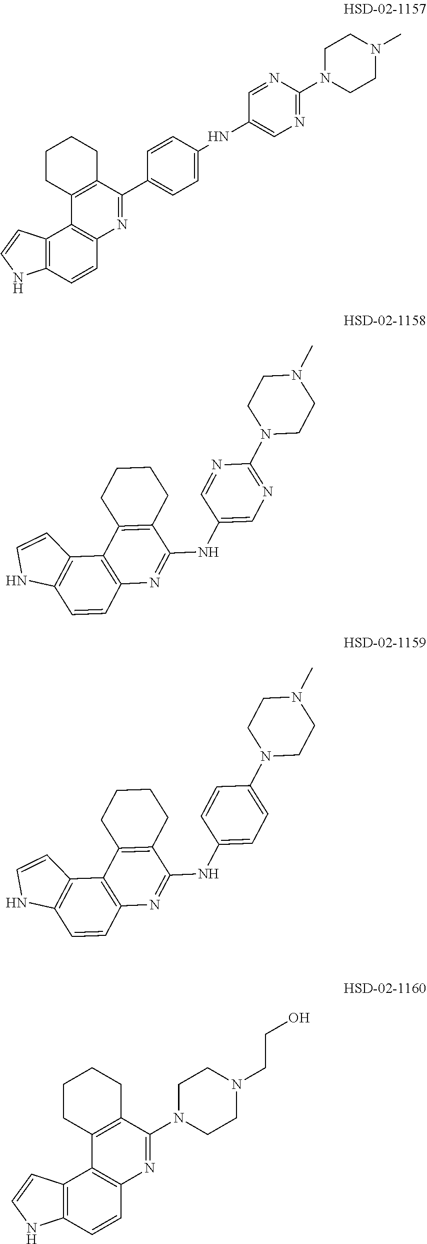 [SMILES: CN1CCN(c2ncc(Nc3ccc(-c4nc5ccc6[nH]ccc6c5c5c4CCCC5)cc3)cn2)CC1 ; CN1CCN(c2ncc(Nc3nc4ccc5[nH]ccc5c4c4c3CCCC4)cn2)CC1 ; CN1CCN(c2ccc(Nc3nc4ccc5[nH]ccc5c4c4c3CCCC4)cc2)CC1 ; OCCN1CCN(c2nc3ccc4[nH]ccc4c3c3c2CCCC3)CC1]

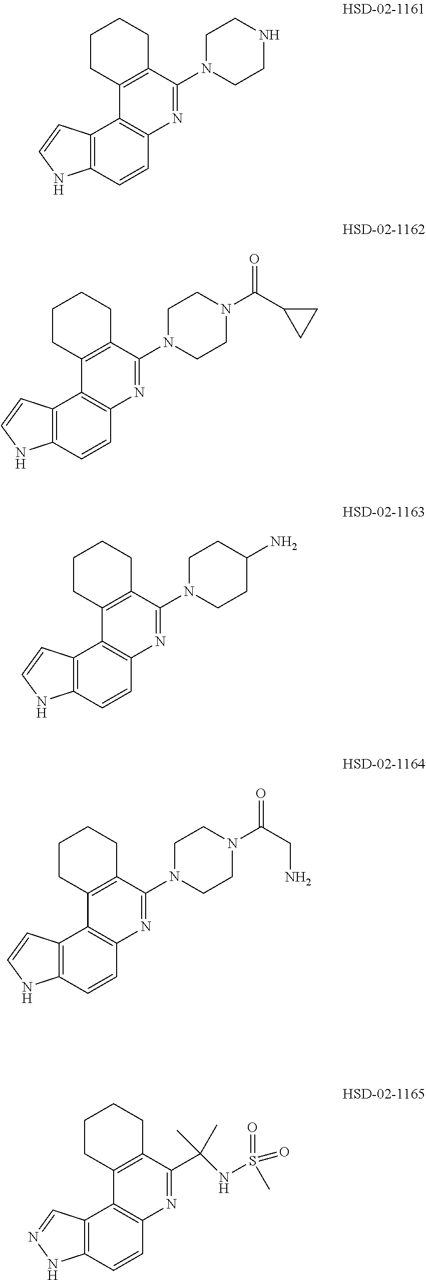 [SMILES: c1cc2c(ccc3nc(N4CCNCC4)c4c(c32)CCCC4)[nH]1 ; O=C(C1CC1)N1CCN(c2nc3ccc4[nH]ccc4c3c3c2CCCC3)CC1 ; NC1CCN(c2nc3ccc4[nH]ccc4c3c3c2CCCC3)CC1 ; NCC(=O)N1CCN(c2nc3ccc4[nH]ccc4c3c3c2CCCC3)CC1 ; CC(C)(NS(C)(=O)=O)c1nc2ccc3[nH]ncc3c2c2c1CCCC2]

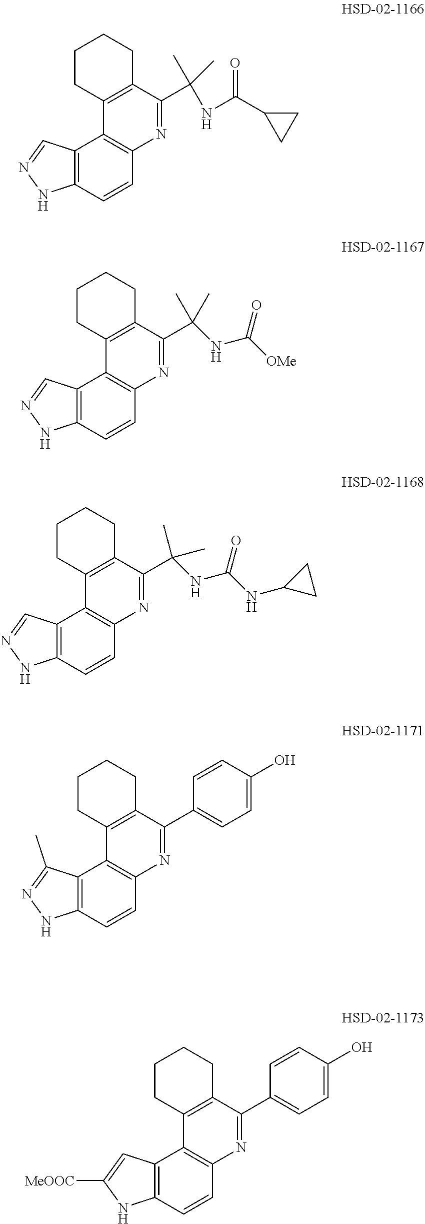 [SMILES: CC(C)(NC(=O)C1CC1)c1nc2ccc3[nH]ncc3c2c2c1CCCC2 ; COC(=O)NC(C)(C)c1nc2ccc3[nH]ncc3c2c2c1CCCC2 ; CC(C)(NC(=O)NC1CC1)c1nc2ccc3[nH]ncc3c2c2c1CCCC2 ; Cc1n[nH]c2ccc3nc(-c4ccc(O)cc4)c4c(c3c12)CCCC4 ; COC(=O)c1cc2c(ccc3nc(-c4ccc(O)cc4)c4c(c32)CCCC4)[nH]1]

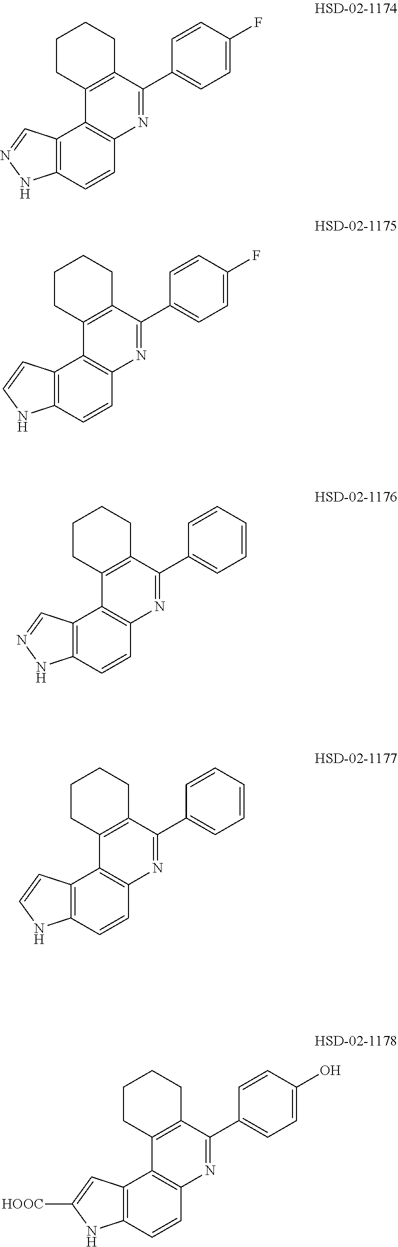 [SMILES: Fc1ccc(-c2nc3ccc4[nH]ncc4c3c3c2CCCC3)cc1 ; Fc1ccc(-c2nc3ccc4[nH]ccc4c3c3c2CCCC3)cc1 ; c1ccc(-c2nc3ccc4[nH]ncc4c3c3c2CCCC3)cc1 ; c1ccc(-c2nc3ccc4[nH]ccc4c3c3c2CCCC3)cc1 ; O=C(O)c1cc2c(ccc3nc(-c4ccc(O)cc4)c4c(c32)CCCC4)[nH]1]

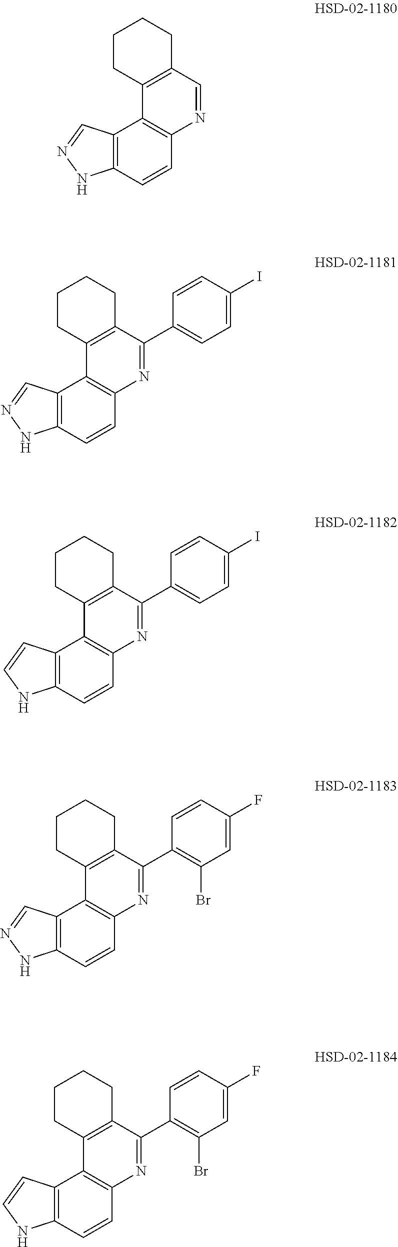 [SMILES: c1nc2ccc3[nH]ncc3c2c2c1CCCC2 ; Ic1ccc(-c2nc3ccc4[nH]ncc4c3c3c2CCCC3)cc1 ; Ic1ccc(-c2nc3ccc4[nH]ccc4c3c3c2CCCC3)cc1 ; Fc1ccc(-c2nc3ccc4[nH]ncc4c3c3c2CCCC3)c(Br)c1 ; Fc1ccc(-c2nc3ccc4[nH]ccc4c3c3c2CCCC3)c(Br)c1]

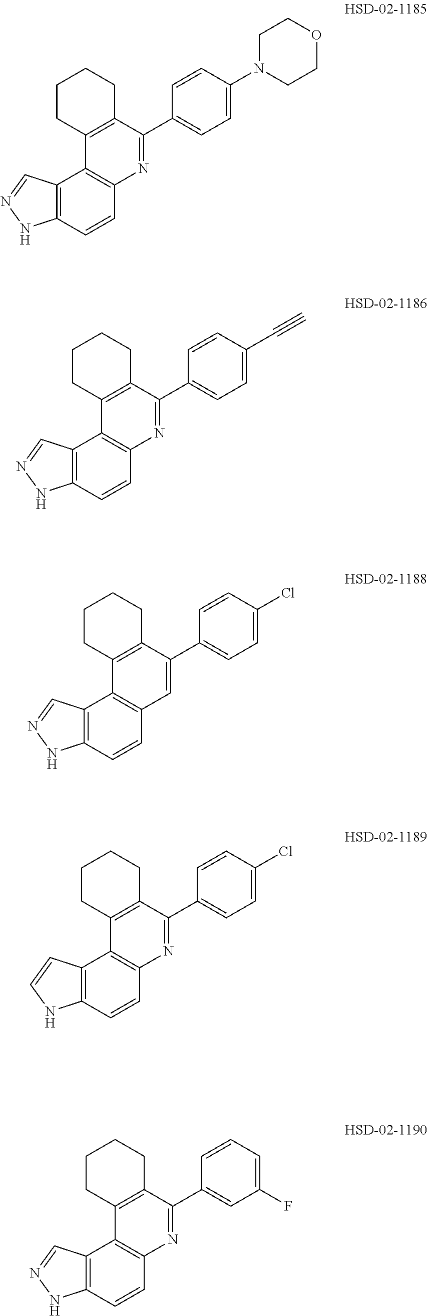 [SMILES: c1cc(N2CCOCC2)ccc1-c1nc2ccc3[nH]ncc3c2c2c1CCCC2 ; C#Cc1ccc(-c2nc3ccc4[nH]ncc4c3c3c2CCCC3)cc1 ; Clc1ccc(-c2cc3ccc4[nH]ncc4c3c3c2CCCC3)cc1 ; Clc1ccc(-c2nc3ccc4[nH]ccc4c3c3c2CCCC3)cc1 ; Fc1cccc(-c2nc3ccc4[nH]ncc4c3c3c2CCCC3)c1]

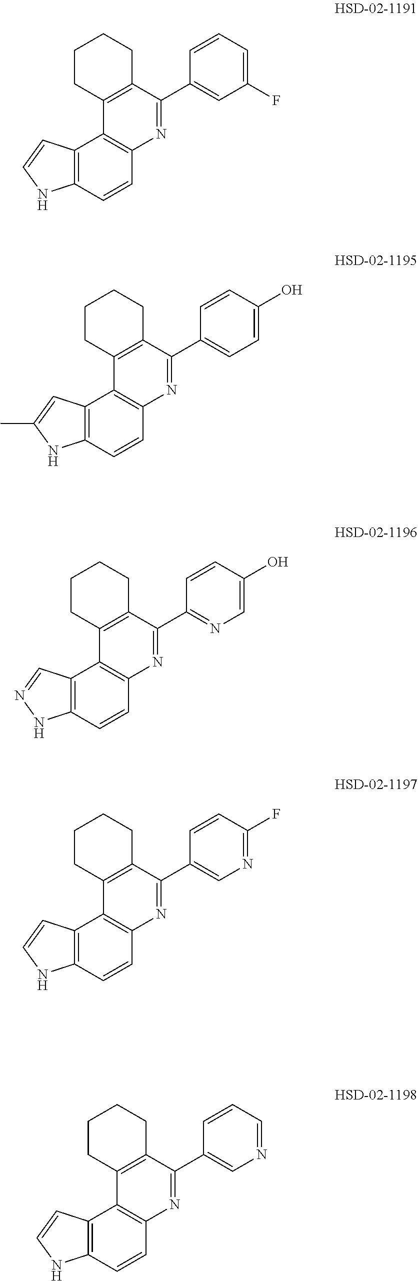 [SMILES: Fc1cccc(-c2nc3ccc4[nH]ccc4c3c3c2CCCC3)c1 ; Cc1cc2c(ccc3nc(-c4ccc(O)cc4)c4c(c32)CCCC4)[nH]1 ; Oc1ccc(-c2nc3ccc4[nH]ncc4c3c3c2CCCC3)nc1 ; Fc1ccc(-c2nc3ccc4[nH]ccc4c3c3c2CCCC3)cn1 ; c1cncc(-c2nc3ccc4[nH]ccc4c3c3c2CCCC3)c1]

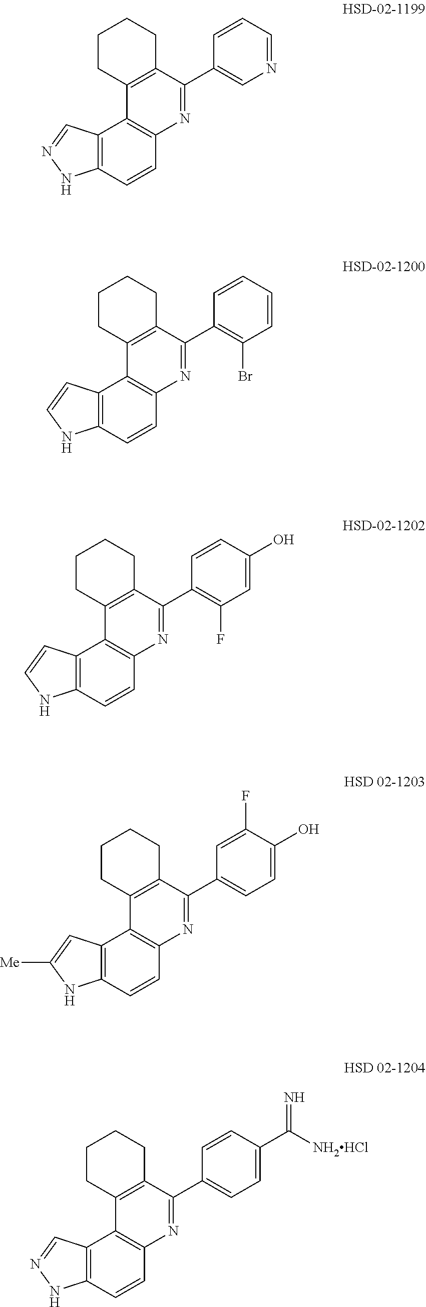

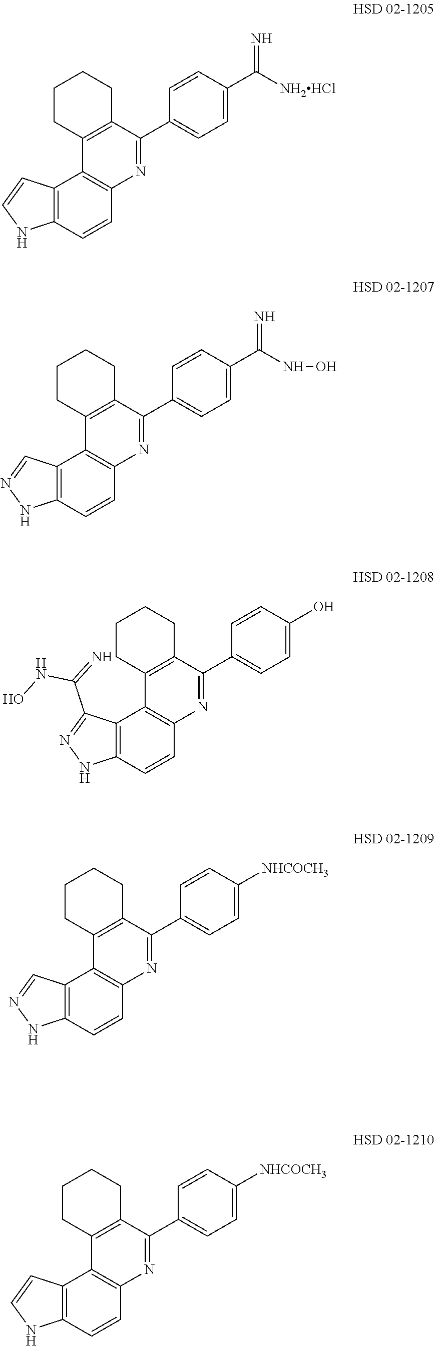

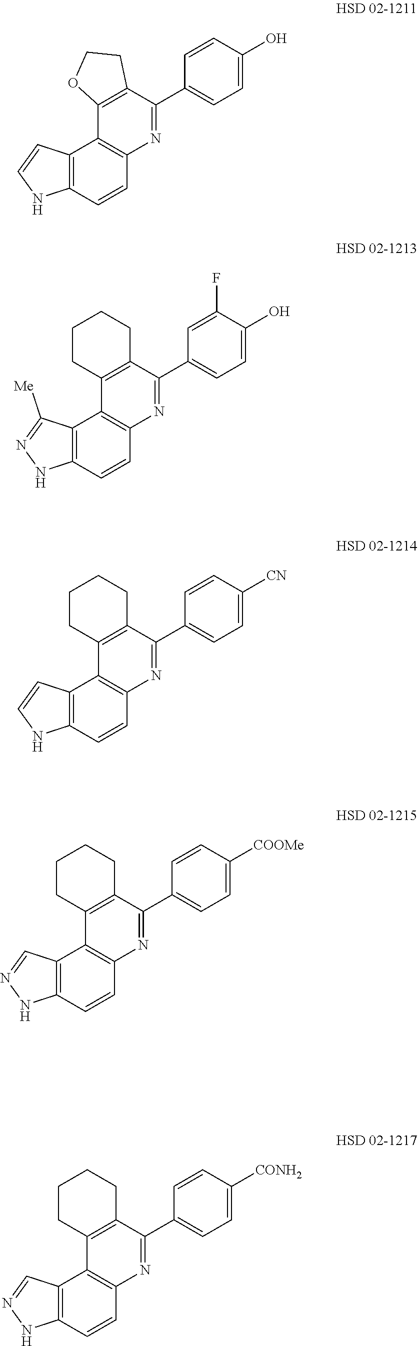 [SMILES: Oc1ccc(-c2nc3ccc4[nH]ccc4c3c3c2CCO3)cc1 ; Cc1n[nH]c2ccc3nc(-c4ccc(O)c(F)c4)c4c(c3c12)CCCC4 ; N#Cc1ccc(-c2nc3ccc4[nH]ccc4c3c3c2CCCC3)cc1 ; COC(=O)c1ccc(-c2nc3ccc4[nH]ncc4c3c3c2CCCC3)cc1 ; NC(=O)c1ccc(-c2nc3ccc4[nH]ncc4c3c3c2CCCC3)cc1]

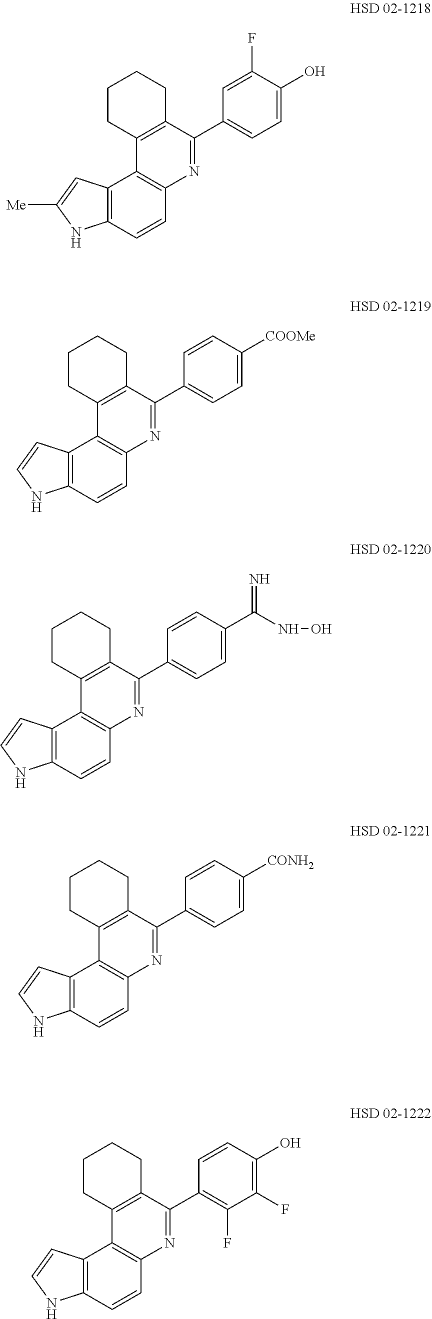 [SMILES: Cc1cc2c(ccc3nc(-c4ccc(O)c(F)c4)c4c(c32)CCCC4)[nH]1 ; COC(=O)c1ccc(-c2nc3ccc4[nH]ccc4c3c3c2CCCC3)cc1 ; N=C(NO)c1ccc(-c2nc3ccc4[nH]ccc4c3c3c2CCCC3)cc1 ; NC(=O)c1ccc(-c2nc3ccc4[nH]ccc4c3c3c2CCCC3)cc1 ; Oc1ccc(-c2nc3ccc4[nH]ccc4c3c3c2CCCC3)c(F)c1F]

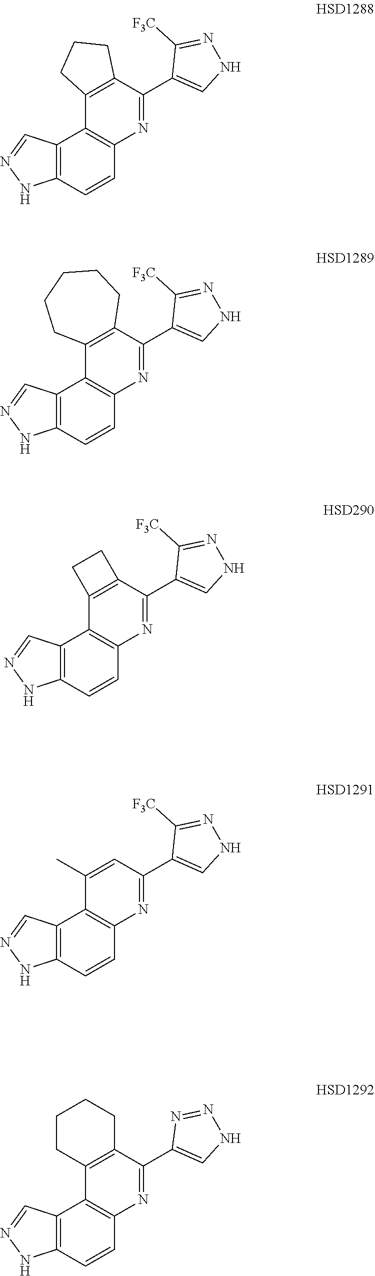 [SMILES: FC(F)(F)c1n[nH]cc1-c1nc2ccc3[nH]ncc3c2c2c1CCC2 ; FC(F)(F)c1n[nH]cc1-c1nc2ccc3[nH]ncc3c2c2c1CCCCC2 ; FC(F)(F)c1n[nH]cc1-c1nc2ccc3[nH]ncc3c2c2c1CC2 ; Cc1cc(-c2c[nH]nc2C(F)(F)F)nc2ccc3[nH]ncc3c12 ; c1[nH]nnc1-c1nc2ccc3[nH]ncc3c2c2c1CCCC2]

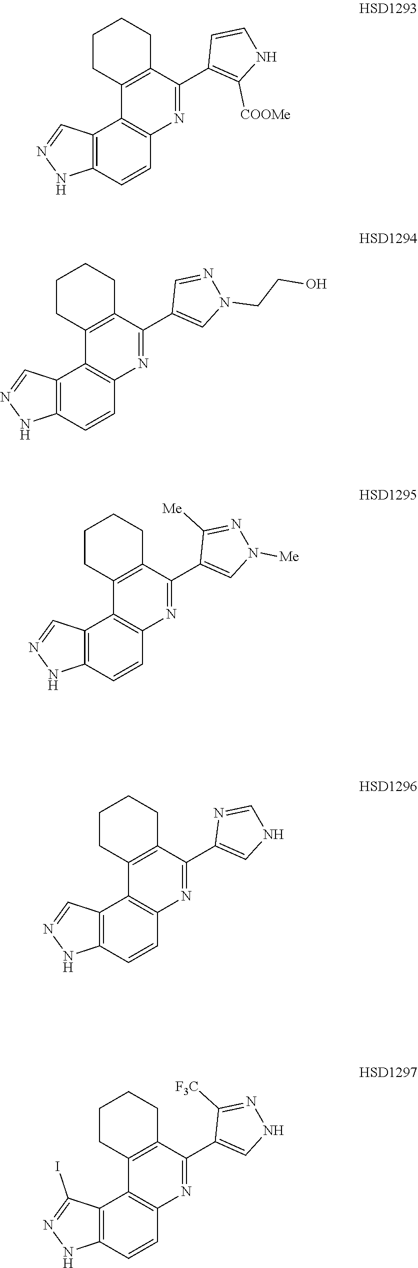 [SMILES: COC(=O)c1[nH]ccc1-c1nc2ccc3[nH]ncc3c2c2c1CCCC2 ; OCCn1cc(-c2nc3ccc4[nH]ncc4c3c3c2CCCC3)cn1 ; Cc1nn(C)cc1-c1nc2ccc3[nH]ncc3c2c2c1CCCC2 ; c1nc(-c2nc3ccc4[nH]ncc4c3c3c2CCCC3)c[nH]1 ; FC(F)(F)c1n[nH]cc1-c1nc2ccc3[nH]nc(I)c3c2c2c1CCCC2]

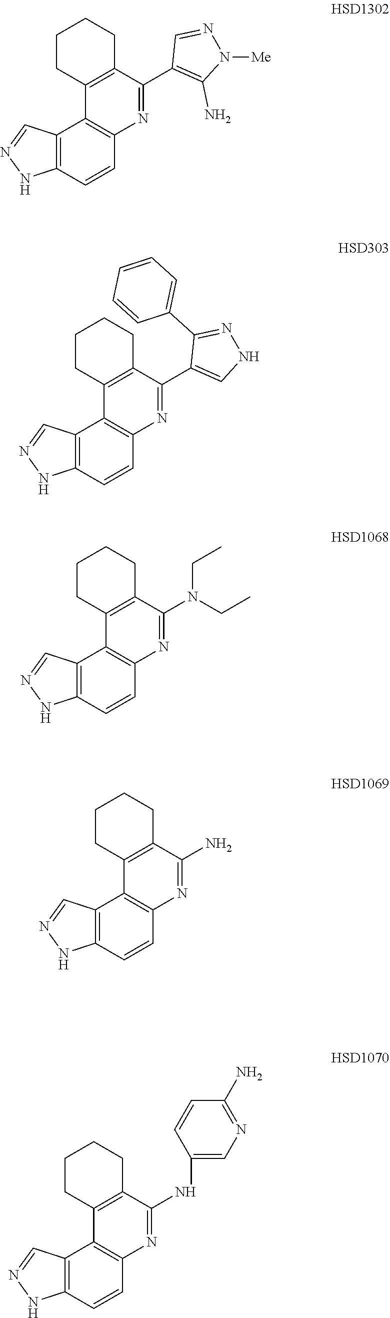 [SMILES: Cn1ncc(-c2nc3ccc4[nH]ncc4c3c3c2CCCC3)c1N ; c1ccc(-c2n[nH]cc2-c2nc3ccc4[nH]ncc4c3c3c2CCCC3)cc1 ; CCN(CC)c1nc2ccc3[nH]ncc3c2c2c1CCCC2 ; Nc1nc2ccc3[nH]ncc3c2c2c1CCCC2 ; Nc1ccc(Nc2nc3ccc4[nH]ncc4c3c3c2CCCC3)cn1]

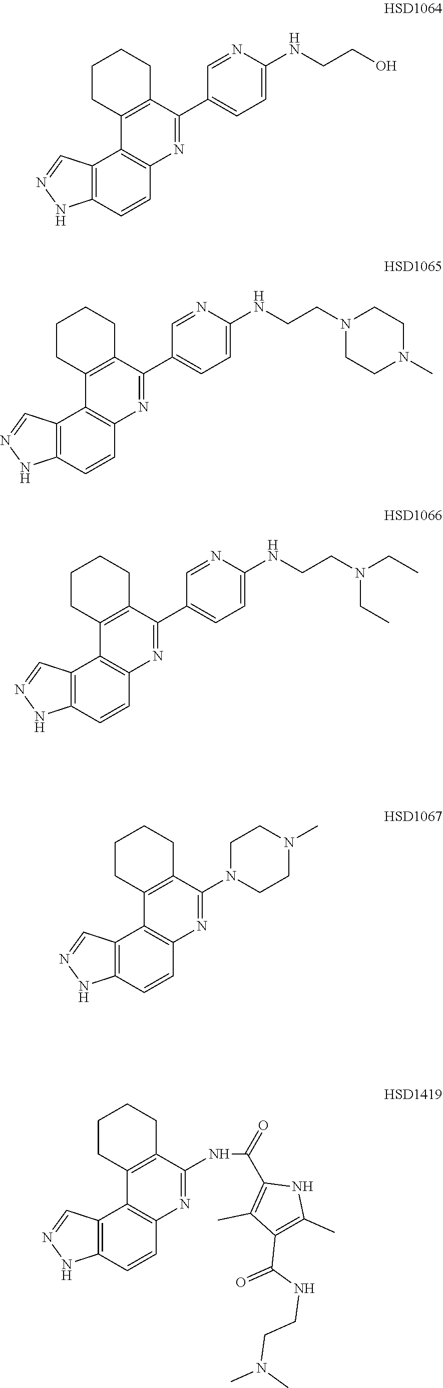 [SMILES: OCCNc1ccc(-c2nc3ccc4[nH]ncc4c3c3c2CCCC3)cn1 ; CN1CCN(CCNc2ccc(-c3nc4ccc5[nH]ncc5c4c4c3CCCC4)cn2)CC1 ; CCN(CC)CCNc1ccc(-c2nc3ccc4[nH]ncc4c3c3c2CCCC3)cn1 ; CN1CCN(c2nc3ccc4[nH]ncc4c3c3c2CCCC3)CC1 ; Cc1[nH]c(C(=O)Nc2nc3ccc4[nH]ncc4c3c3c2CCCC3)c(C)c1C(=O)NCCN(C)C]

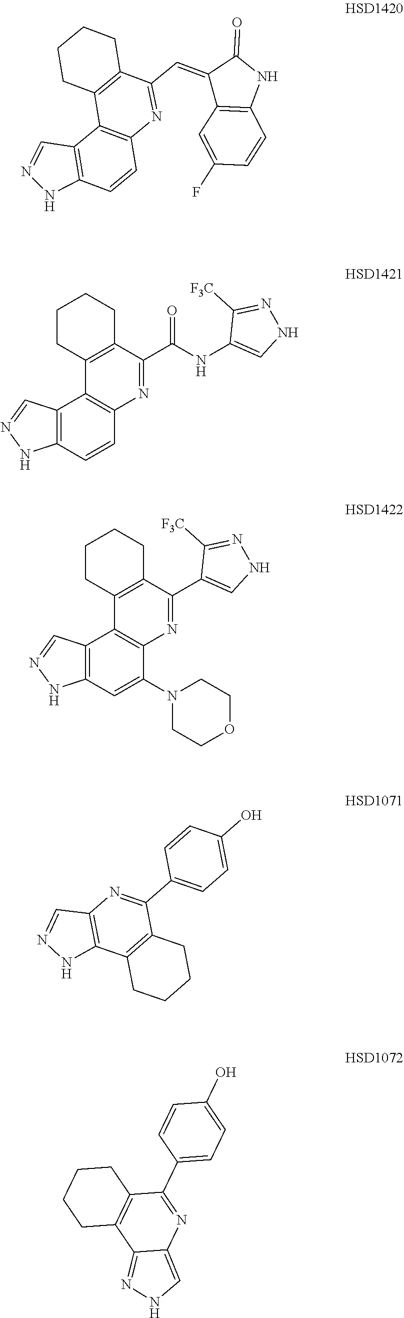 [SMILES: O=C1Nc2ccc(F)cc2/C1=C\c1nc2ccc3[nH]ncc3c2c2c1CCCC2 ; O=C(Nc1c[nH]nc1C(F)(F)F)c1nc2ccc3[nH]ncc3c2c2c1CCCC2 ; FC(F)(F)c1n[nH]cc1-c1nc2c(N3CCOCC3)cc3[nH]ncc3c2c2c1CCCC2 ; Oc1ccc(-c2nc3cn[nH]c3c3c2CCCC3)cc1 ; Oc1ccc(-c2nc3c[nH]nc3c3c2CCCC3)cc1]

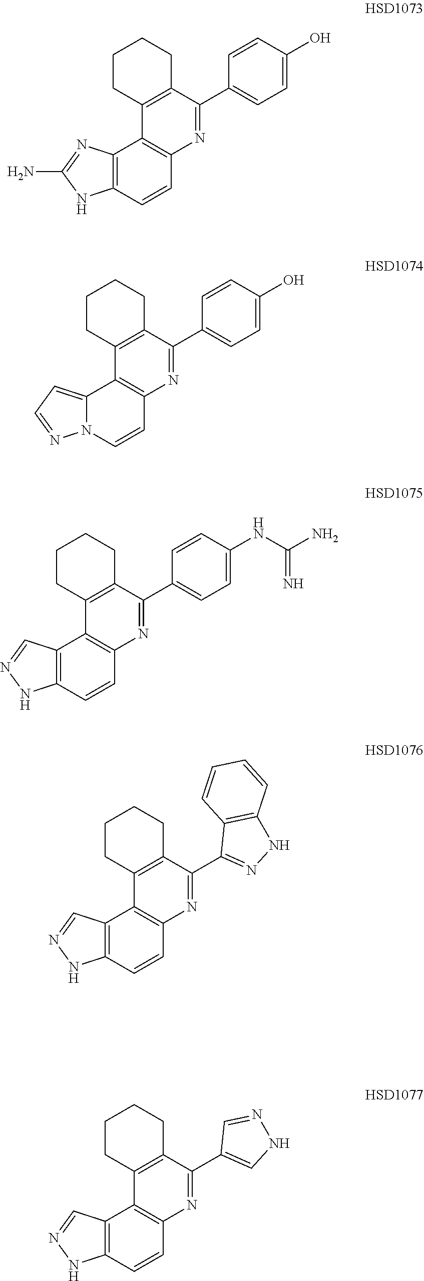 [SMILES: Nc1nc2c(ccc3nc(-c4ccc(O)cc4)c4c(c32)CCCC4)[nH]1 ; Oc1ccc(-c2nc3ccn4nccc4c3c3c2CCCC3)cc1 ; N=C(N)Nc1ccc(-c2nc3ccc4[nH]ncc4c3c3c2CCCC3)cc1 ; c1ccc2c(-c3nc4ccc5[nH]ncc5c4c4c3CCCC4)n[nH]c2c1 ; c1n[nH]cc1-c1nc2ccc3[nH]ncc3c2c2c1CCCC2]

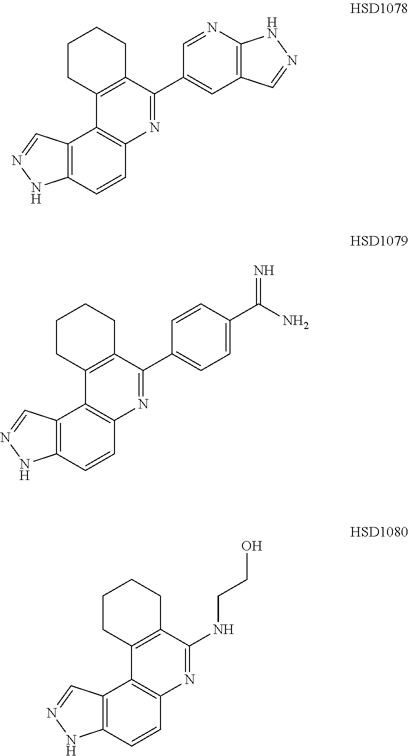 [SMILES: c1nc2[nH]ncc2cc1-c1nc2ccc3[nH]ncc3c2c2c1CCCC2 ; N=C(N)c1ccc(-c2nc3ccc4[nH]ncc4c3c3c2CCCC3)cc1 ; OCCNc1nc2ccc3[nH]ncc3c2c2c1CCCC2]

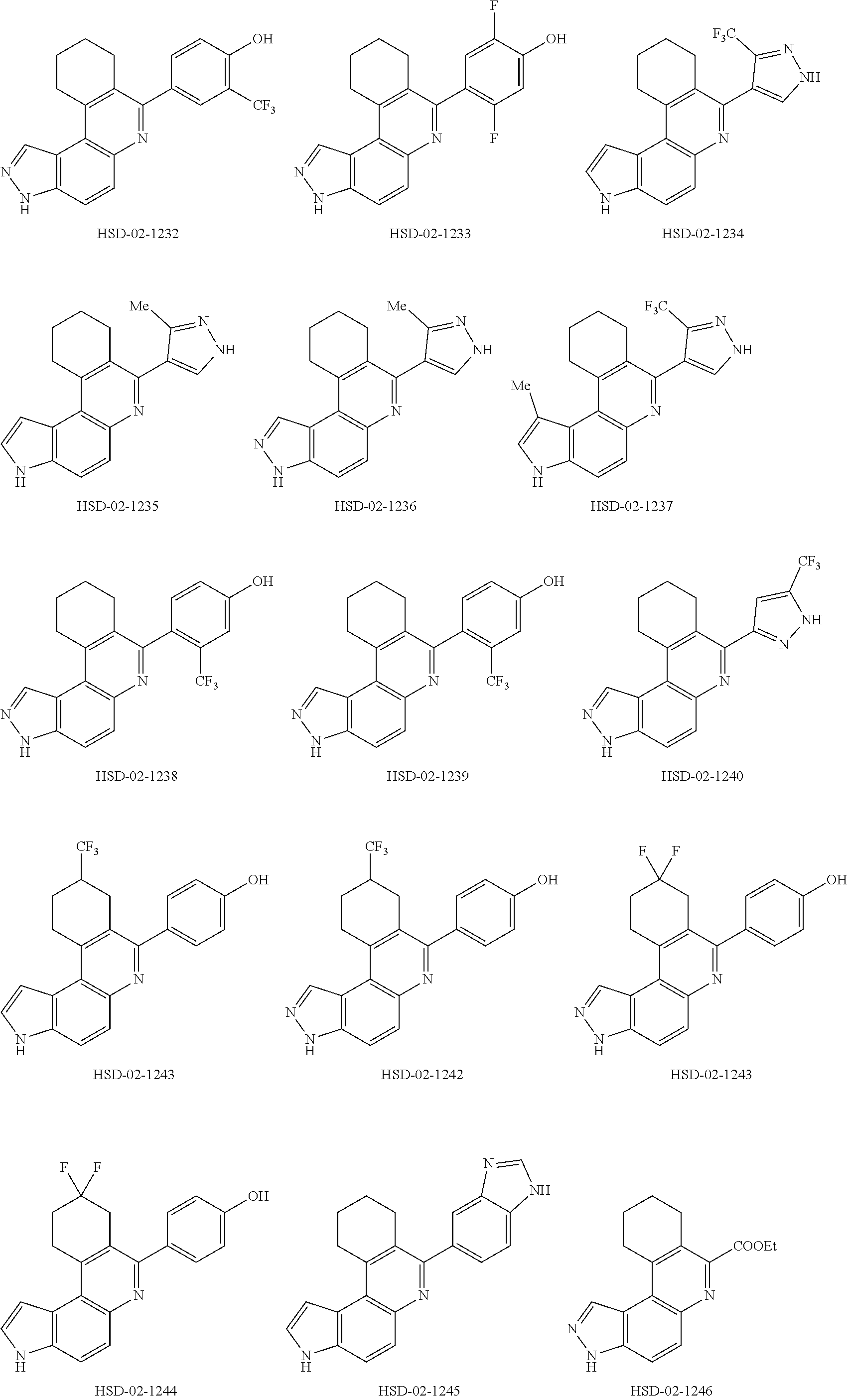

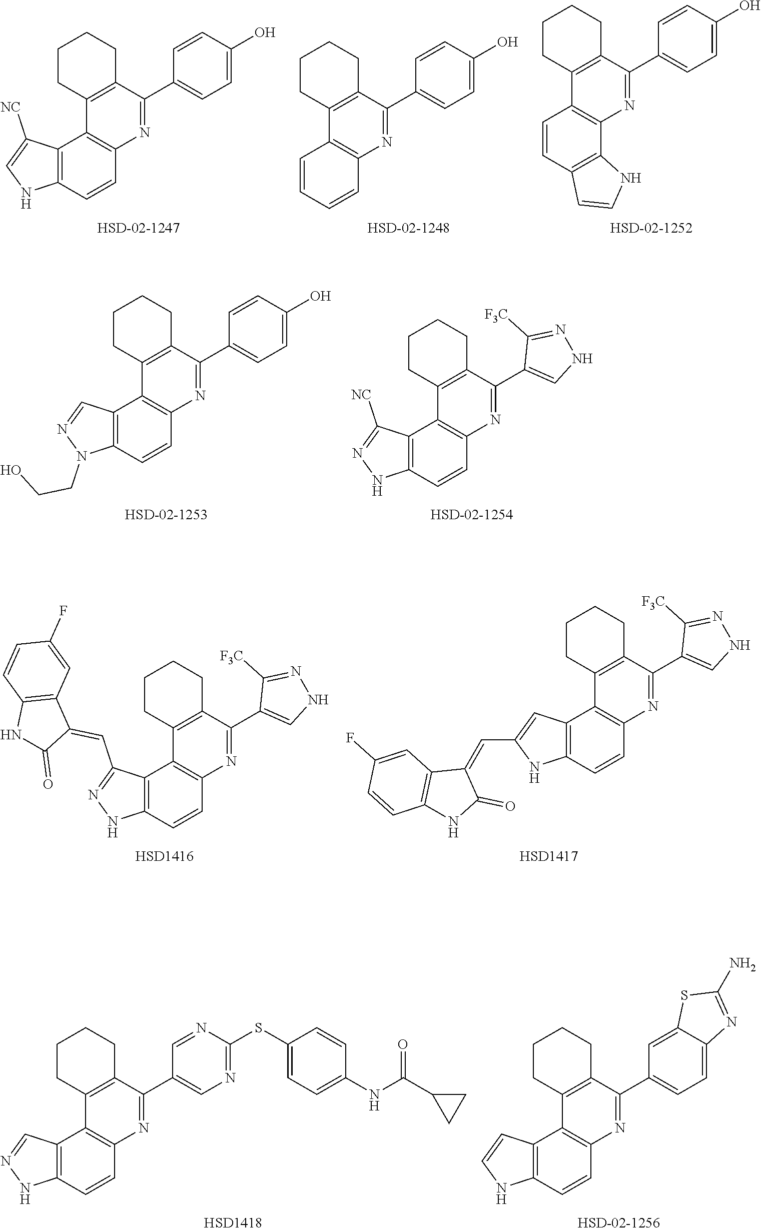

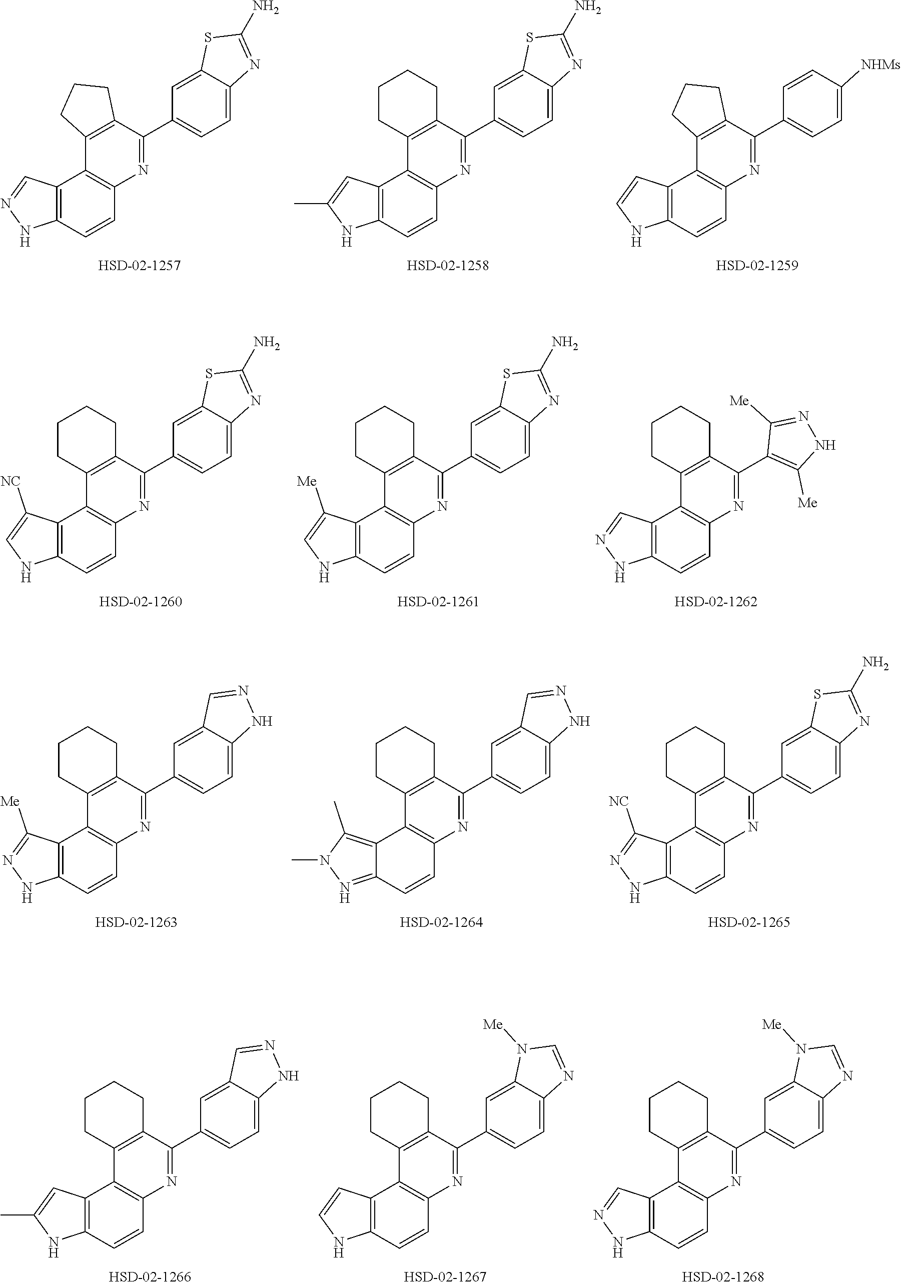

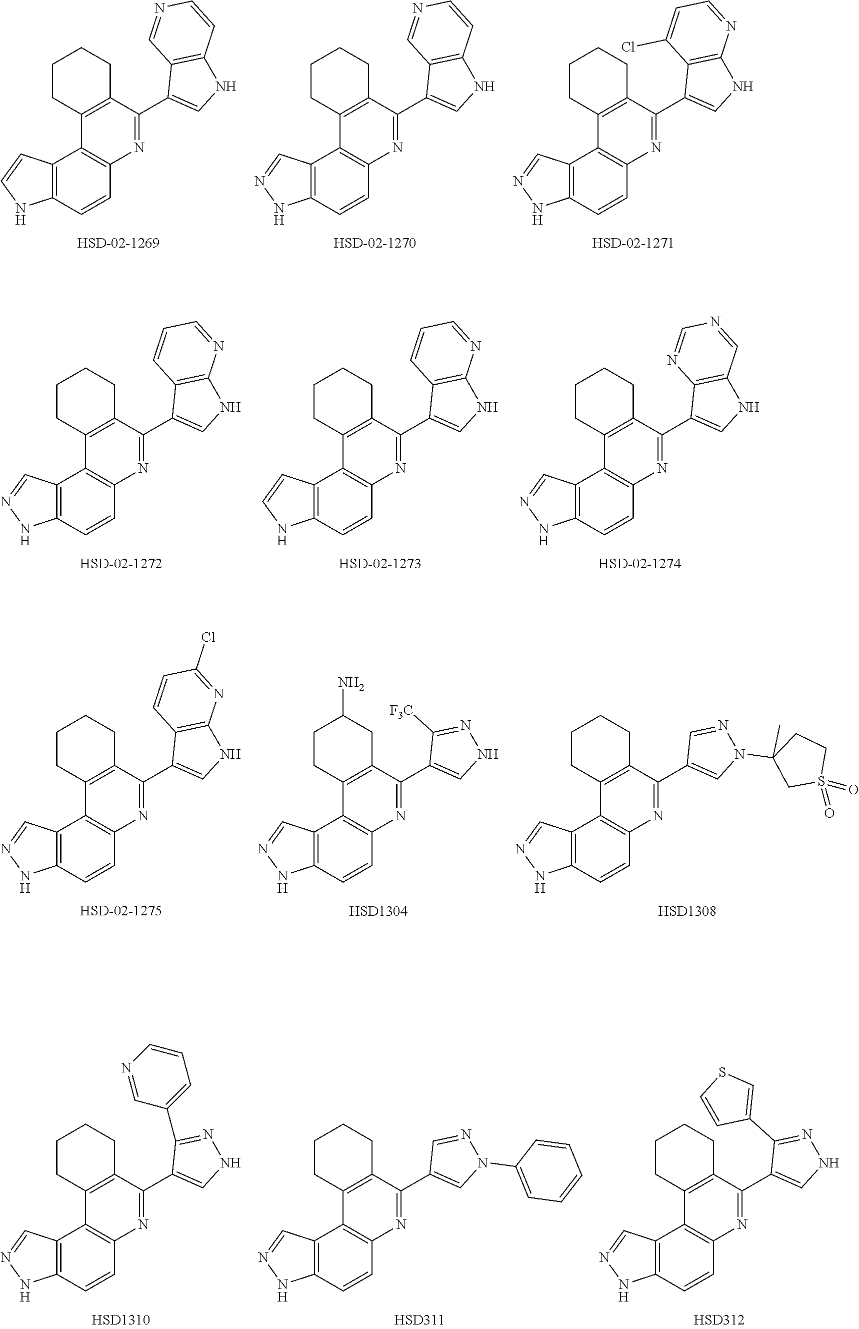

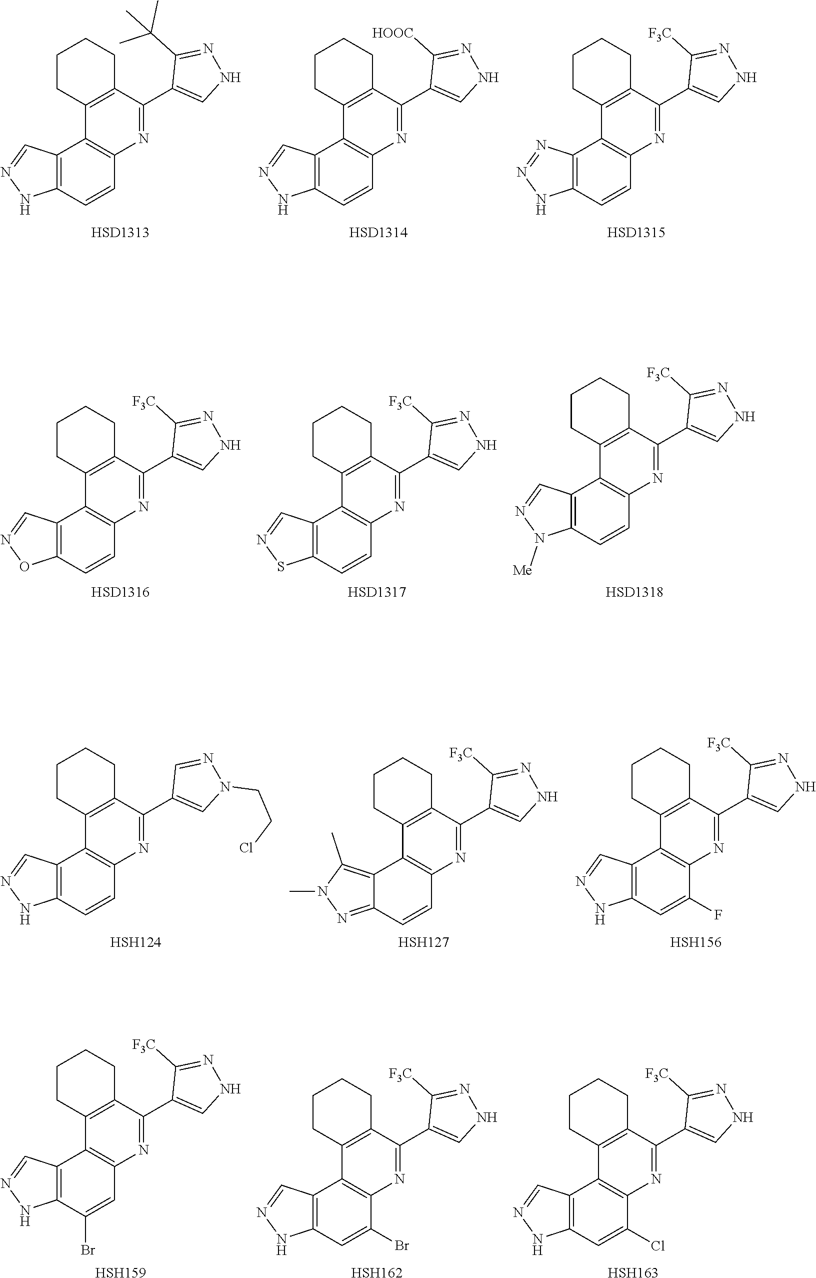

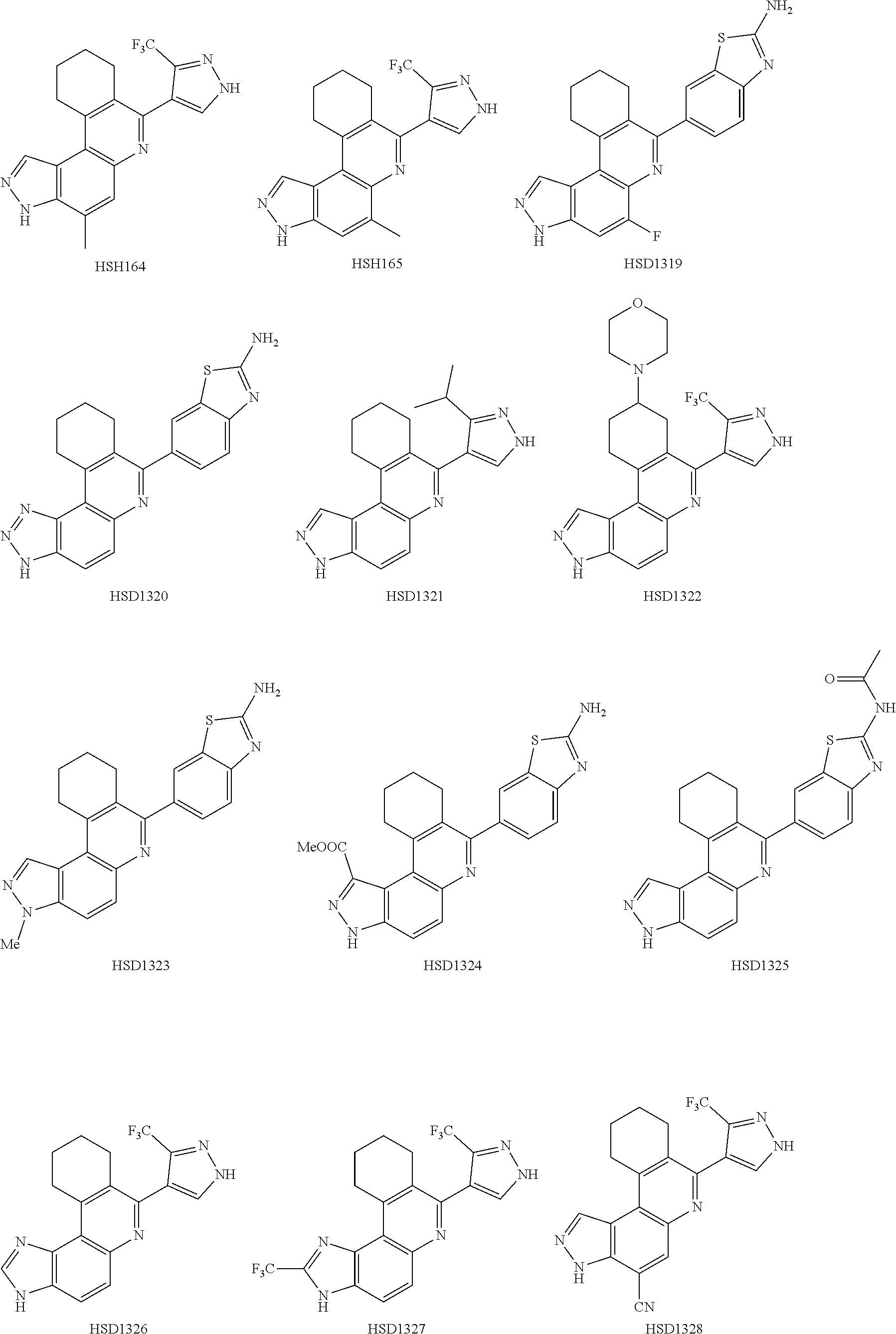

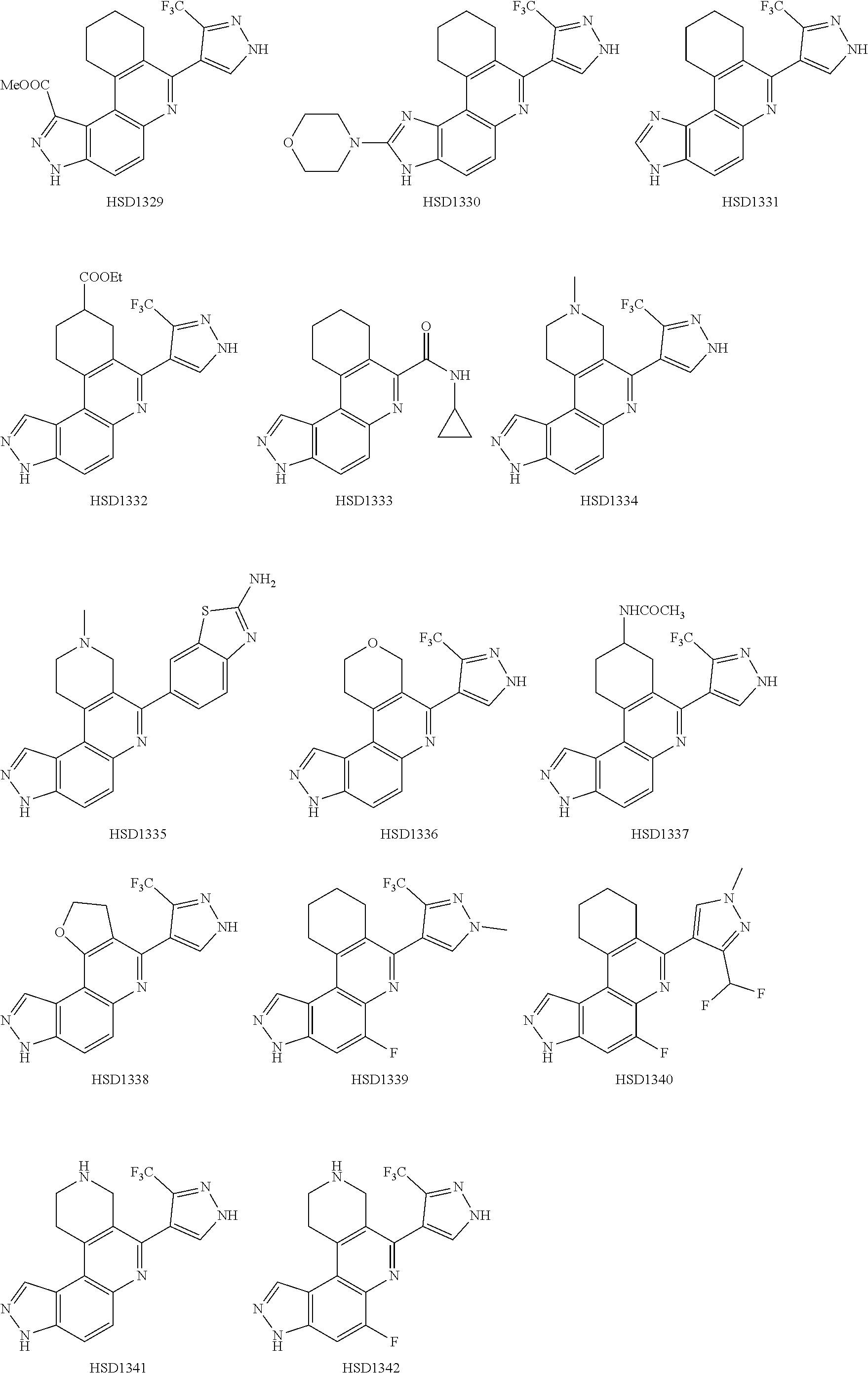

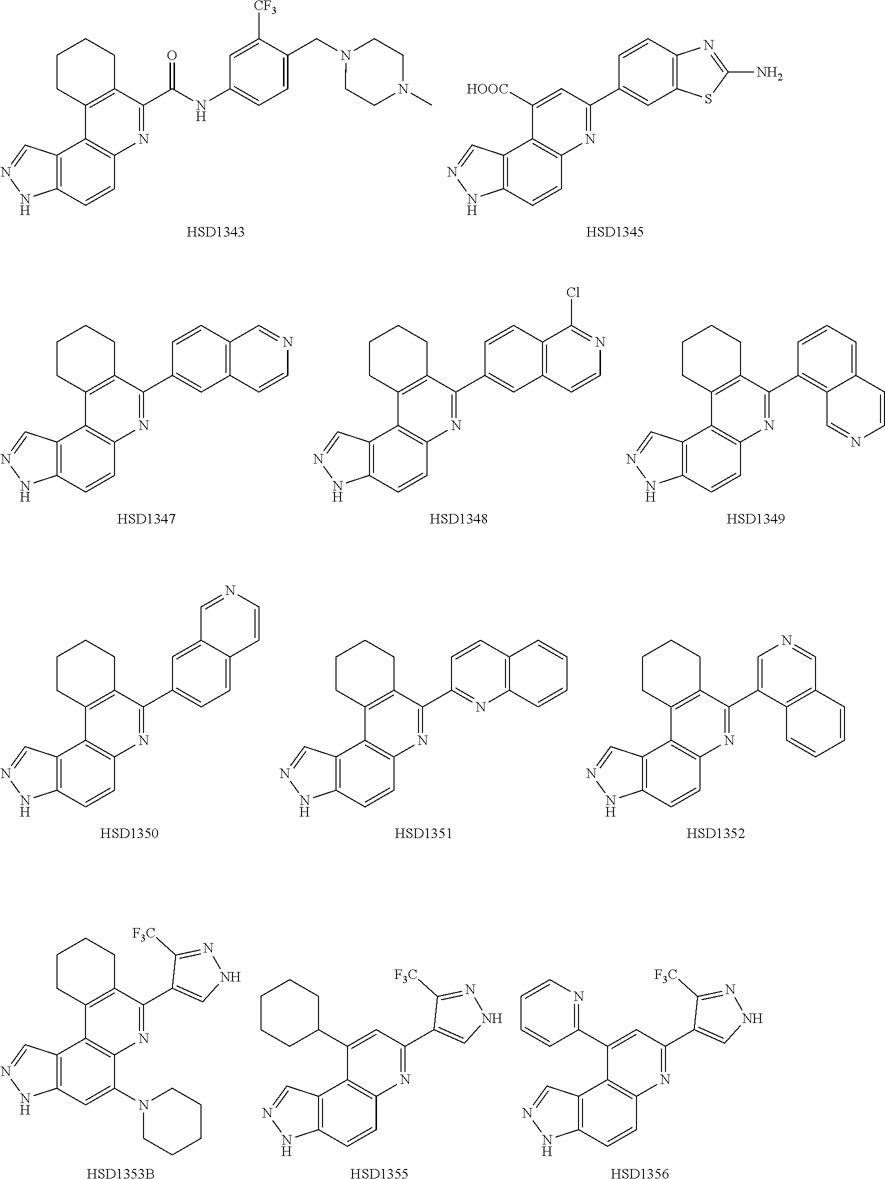

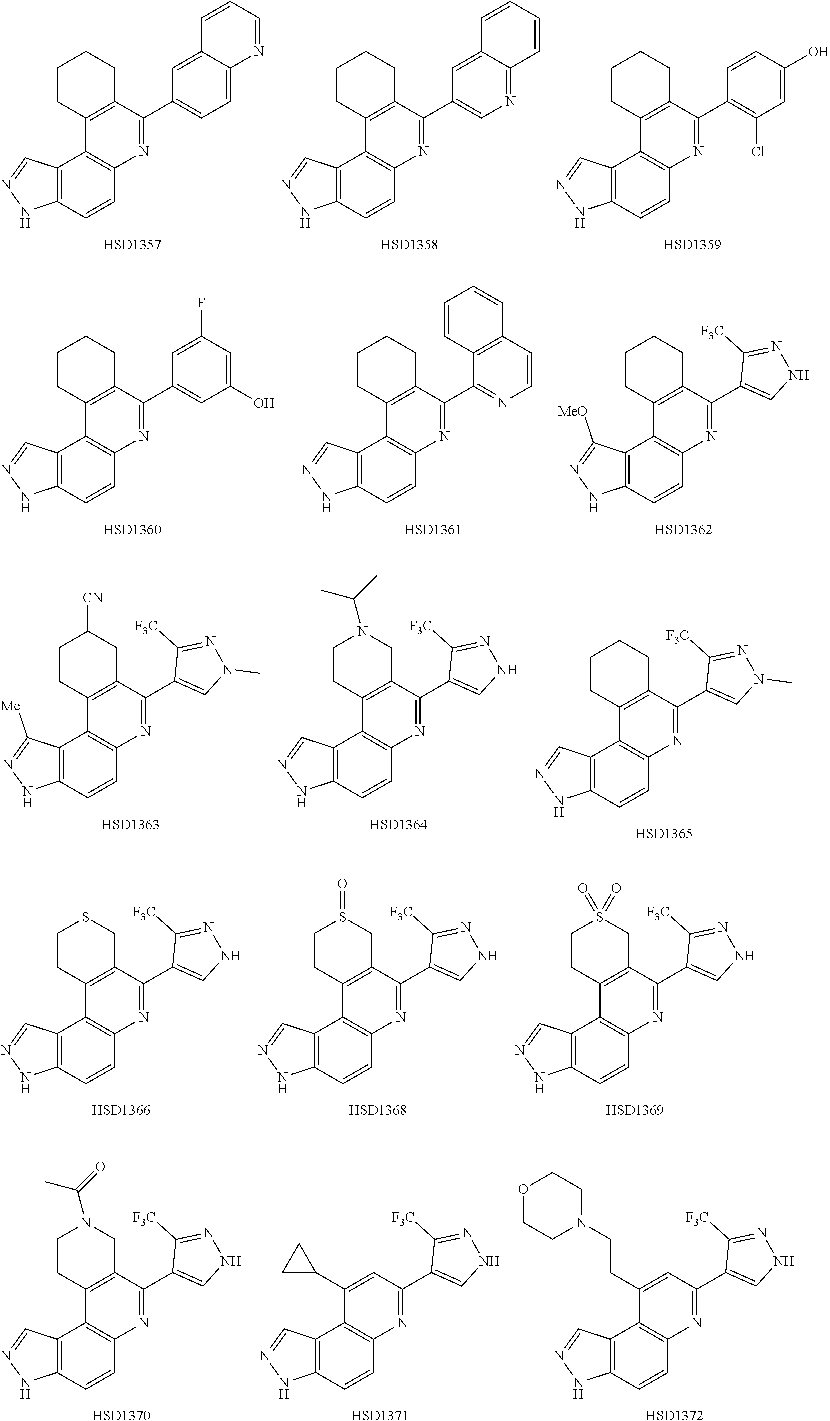

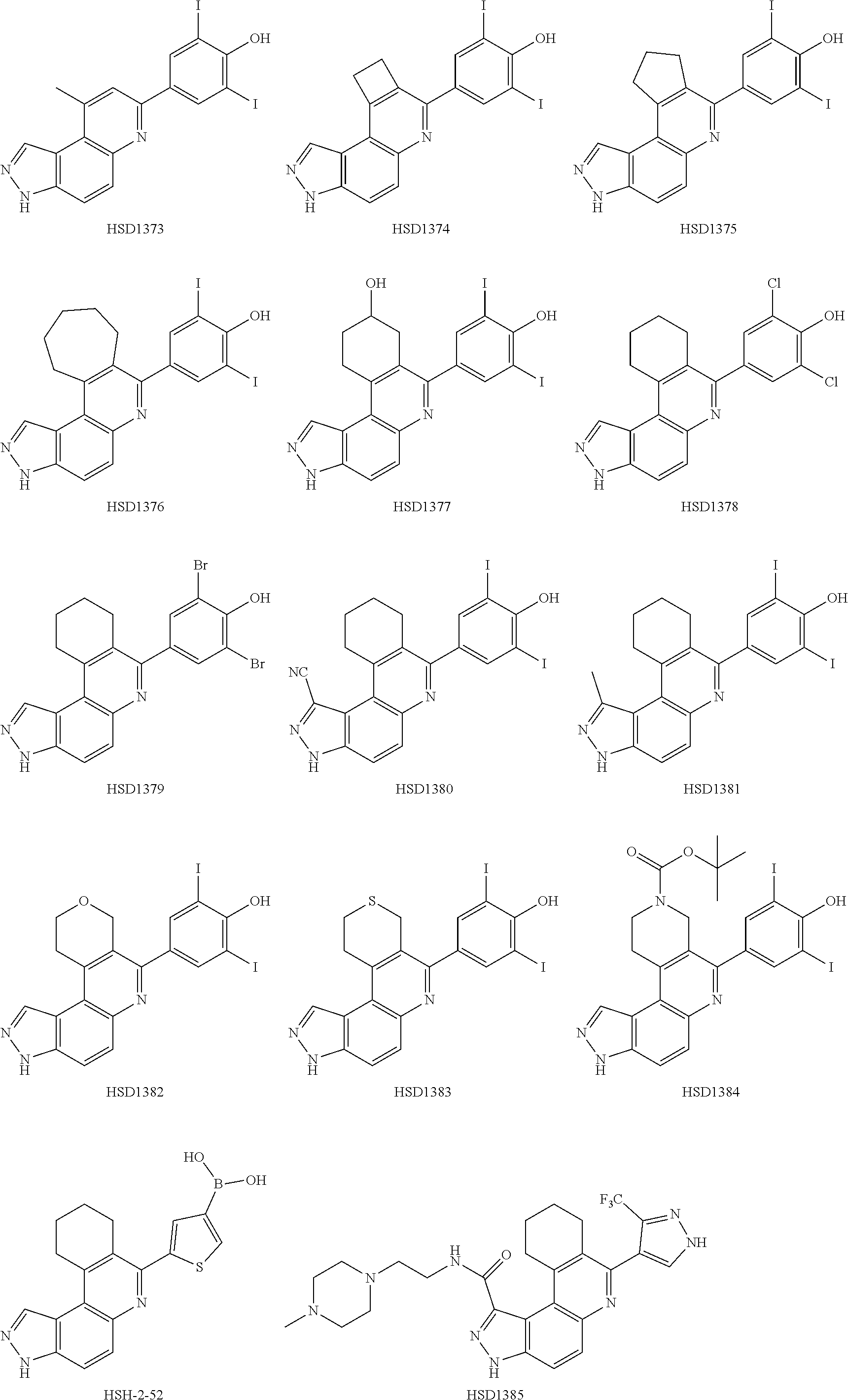

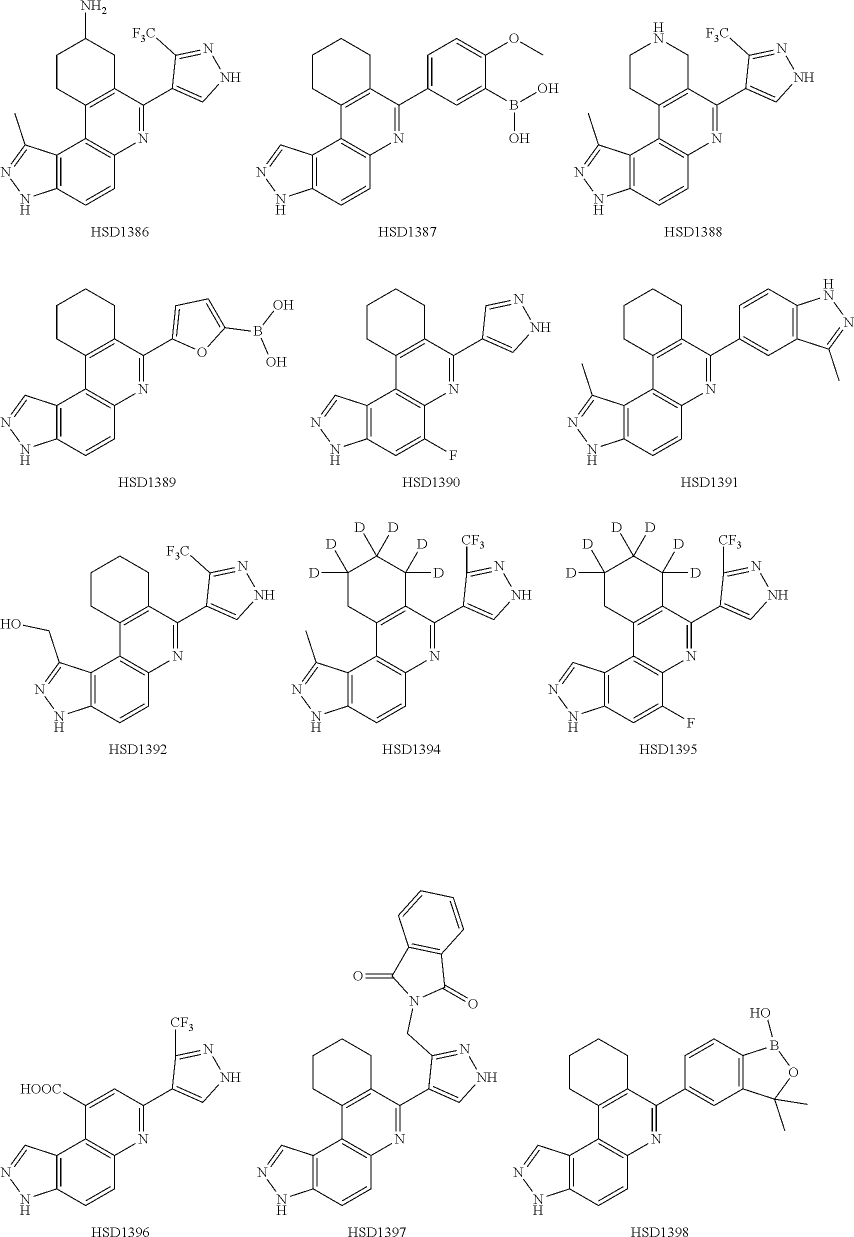

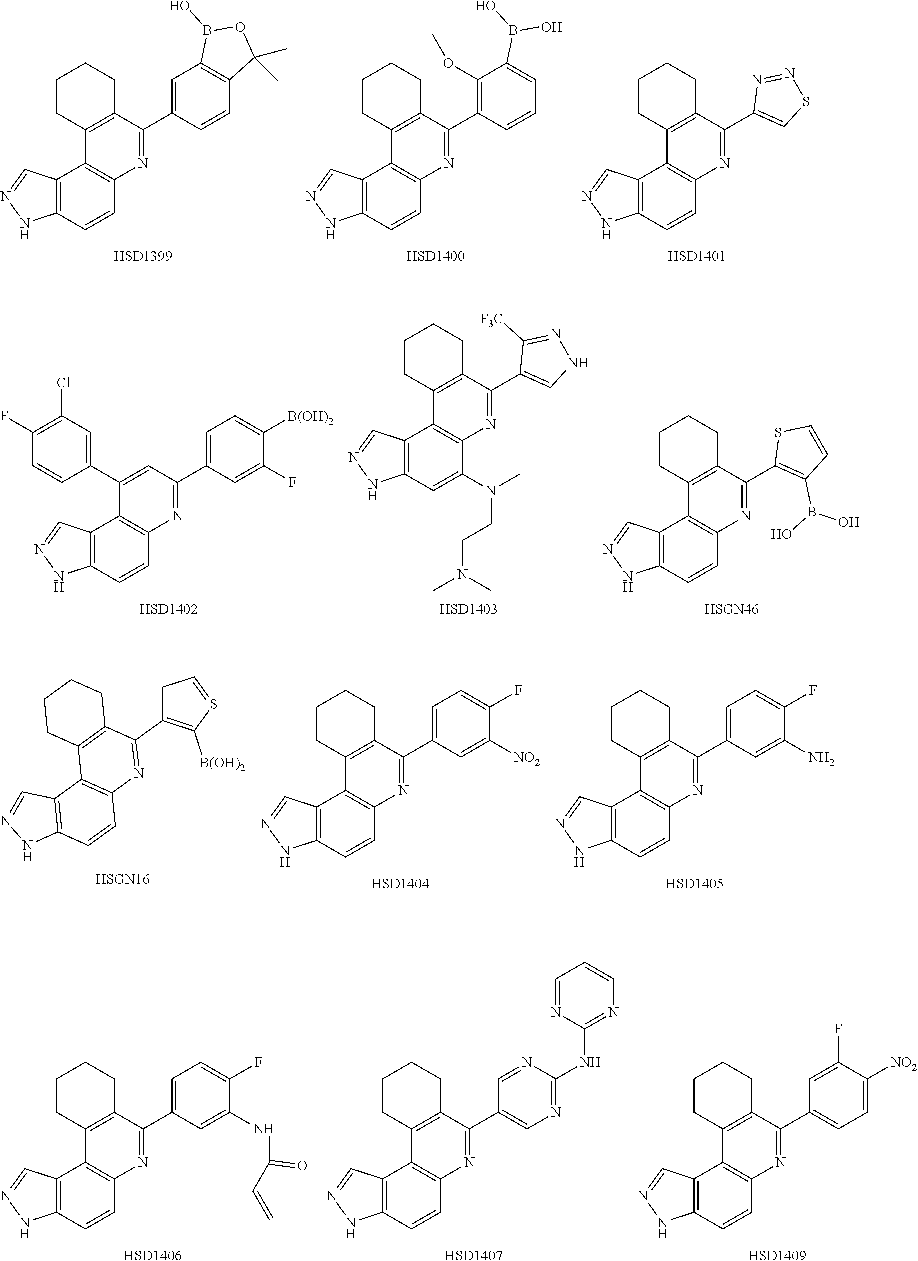

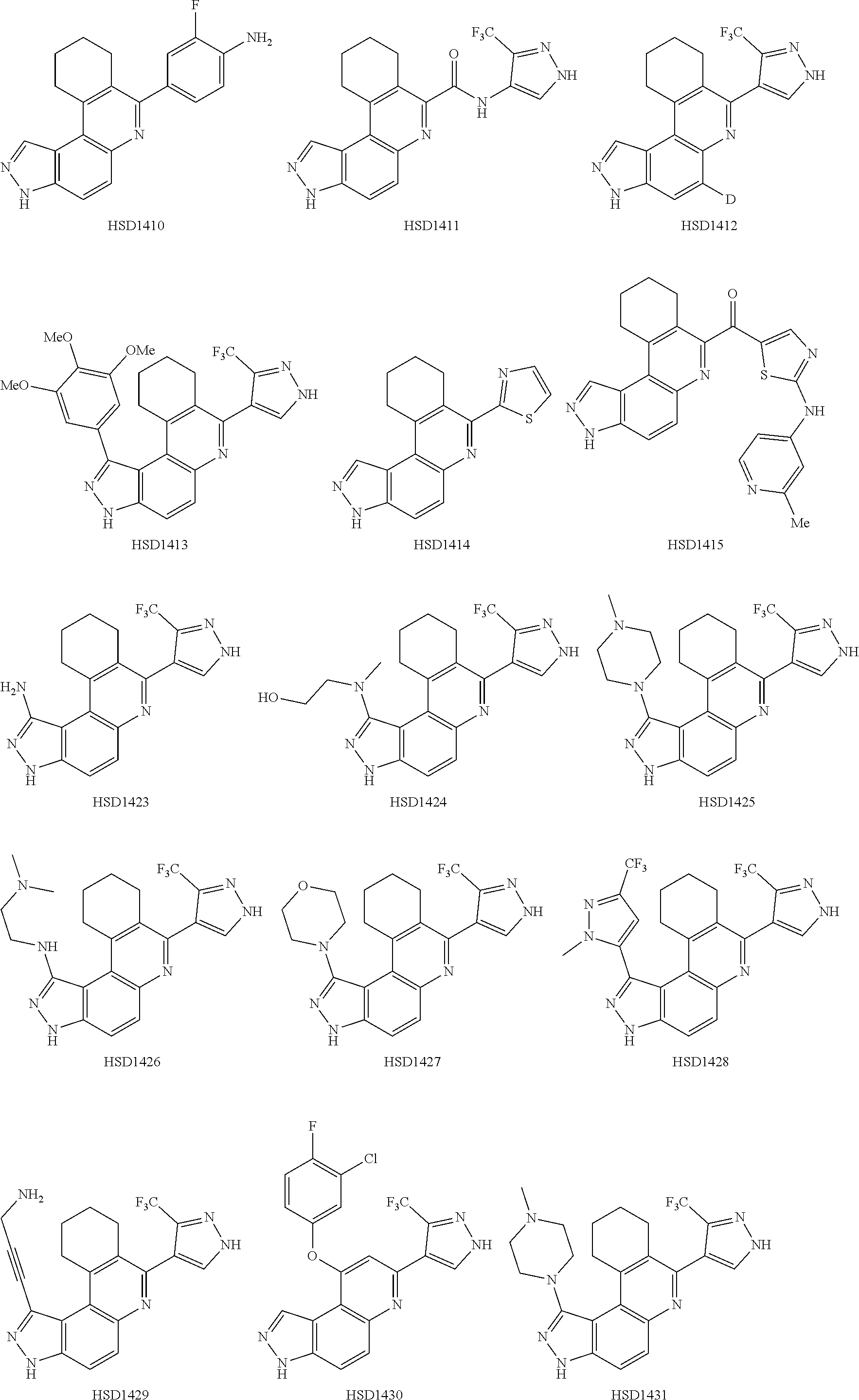

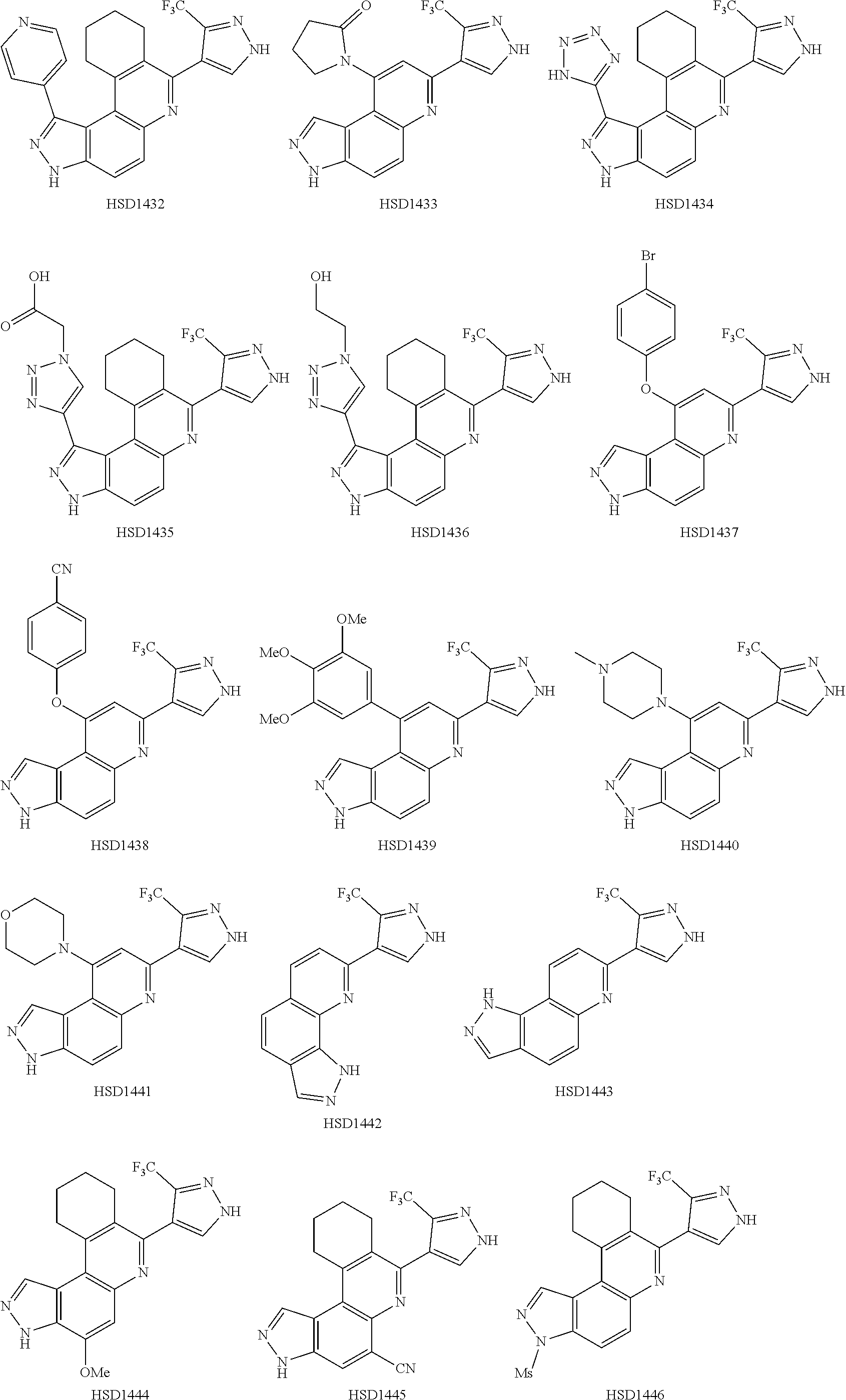

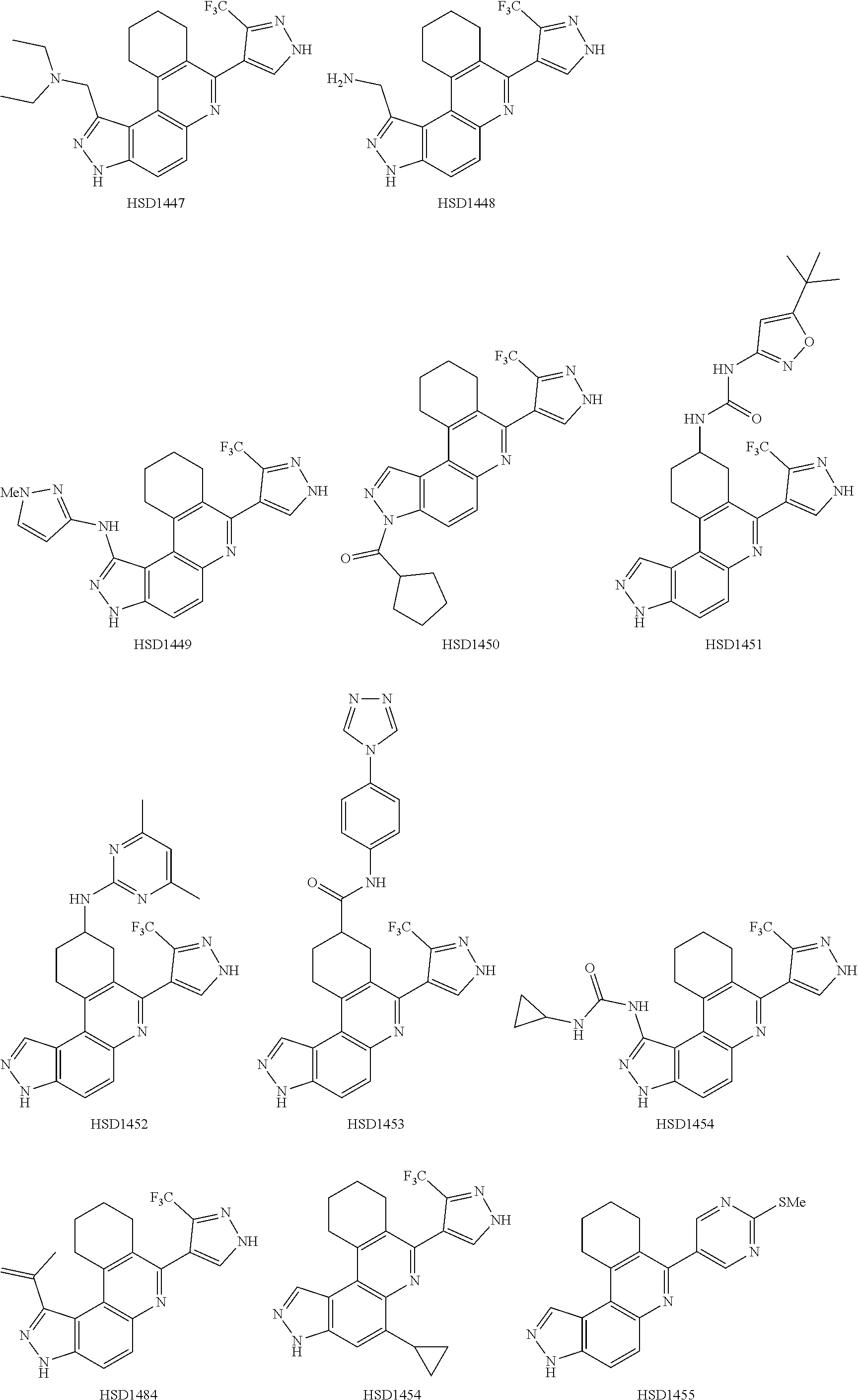

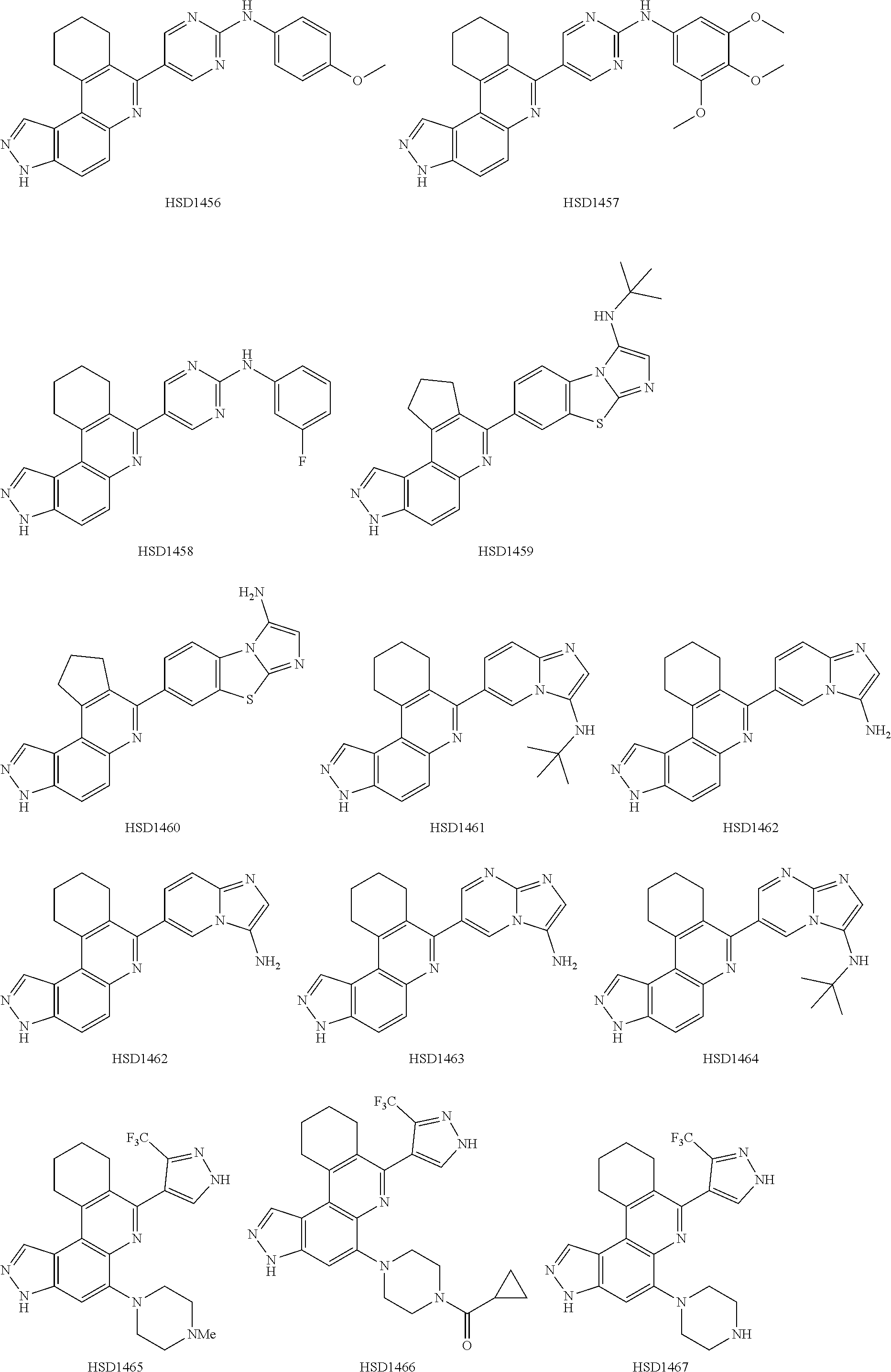

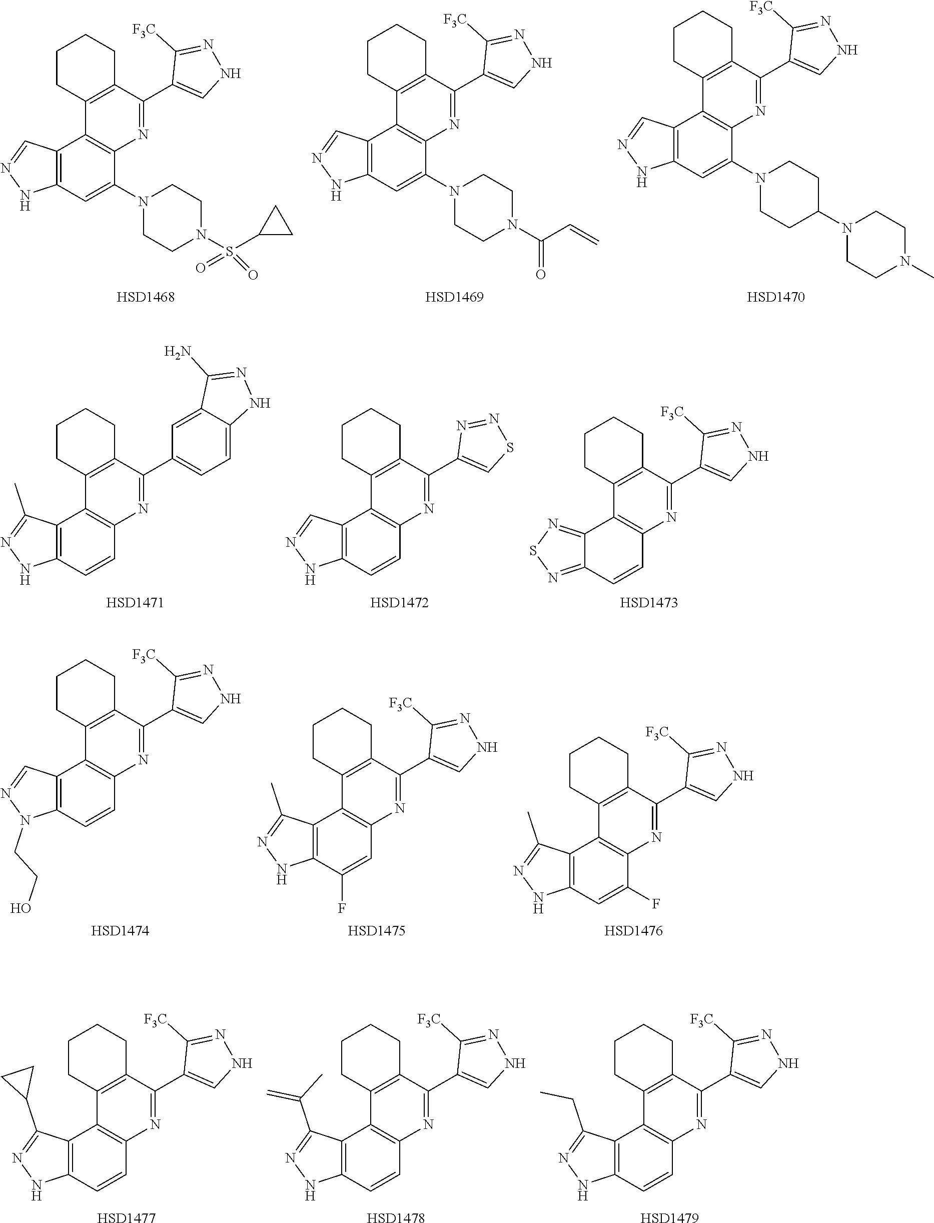

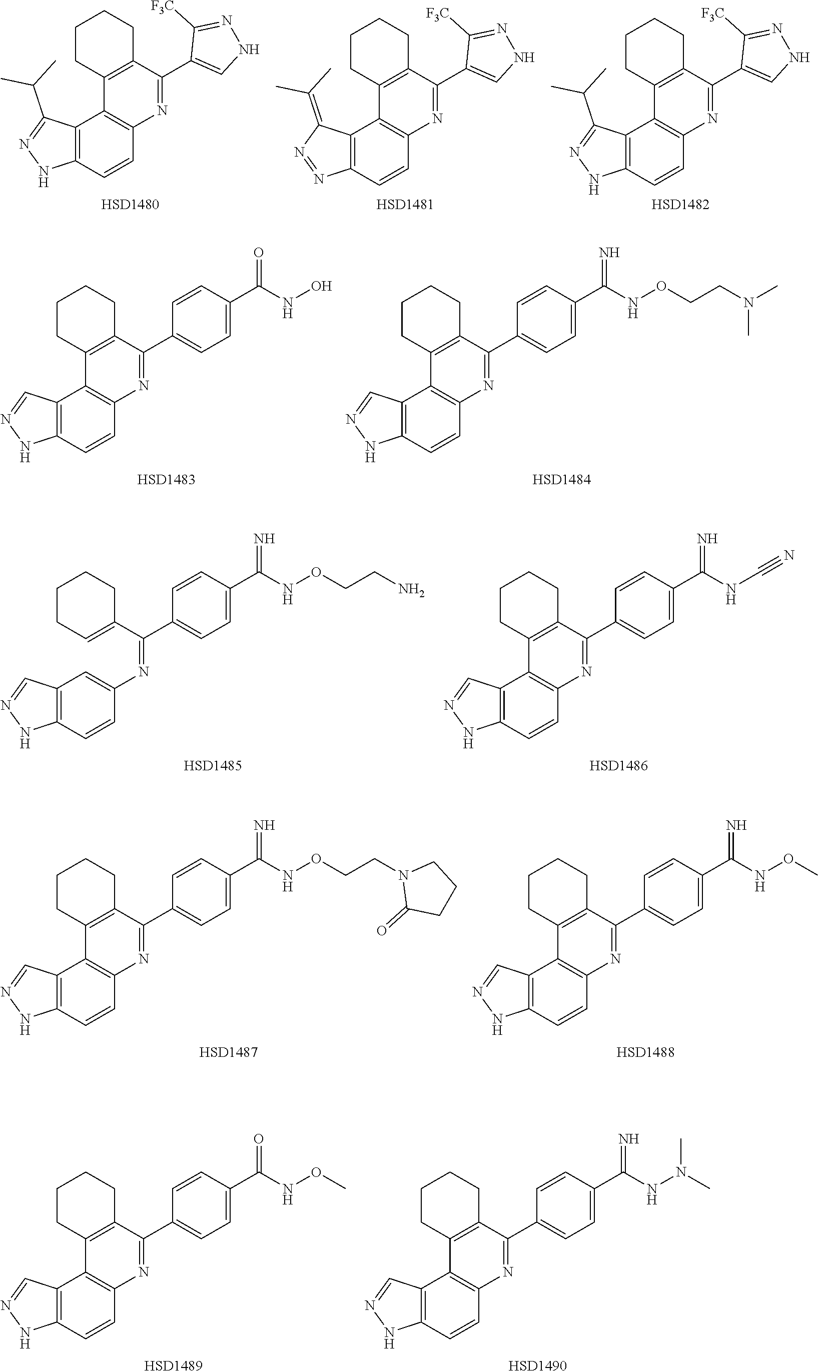

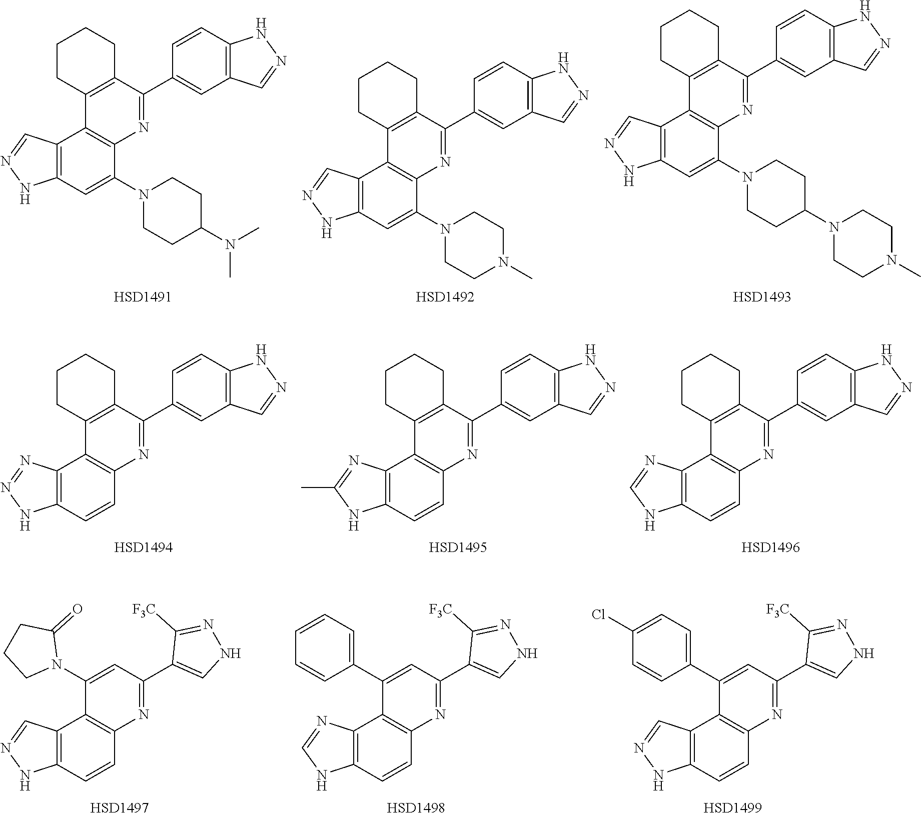

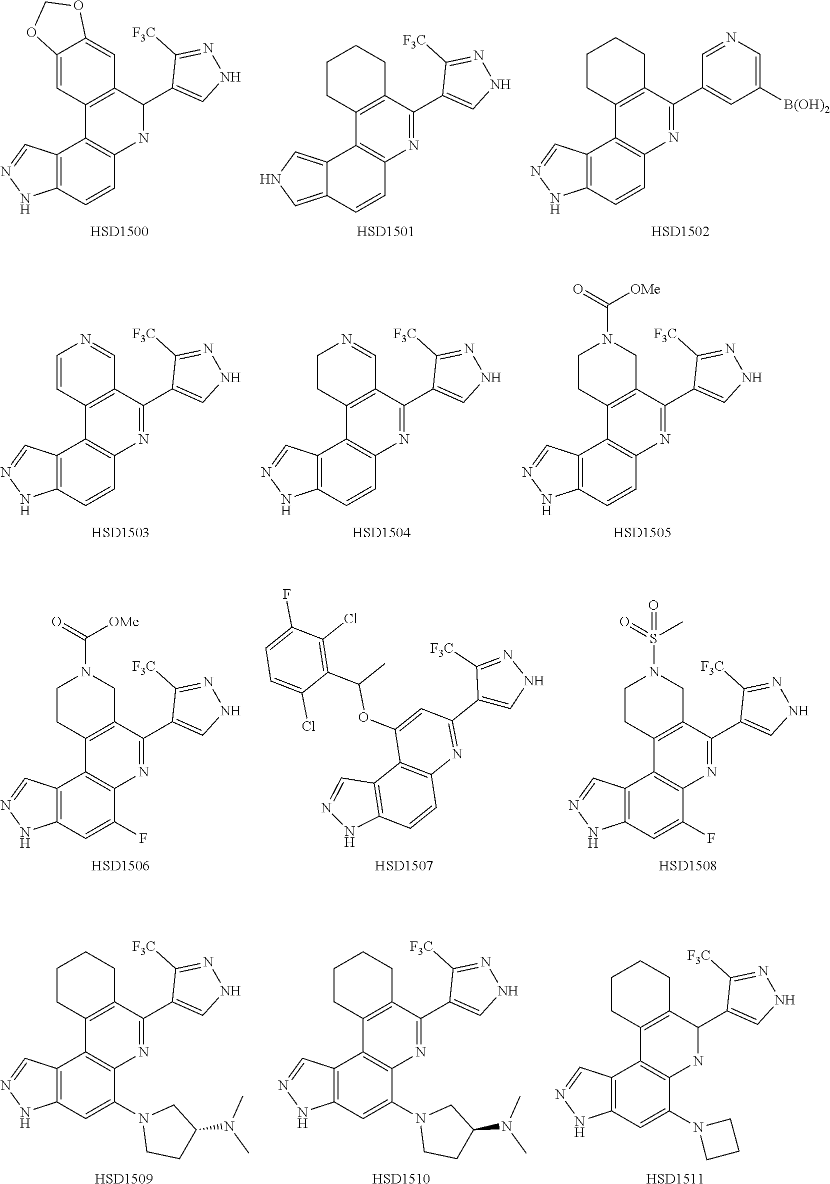

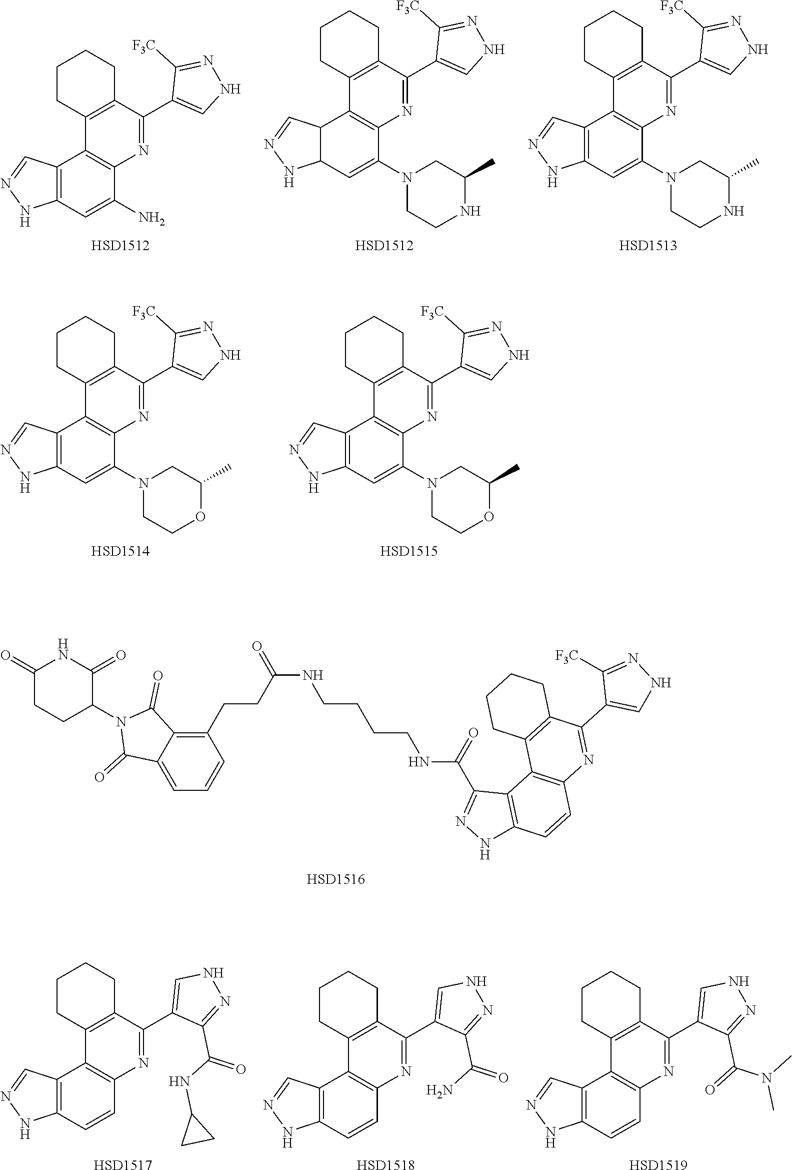

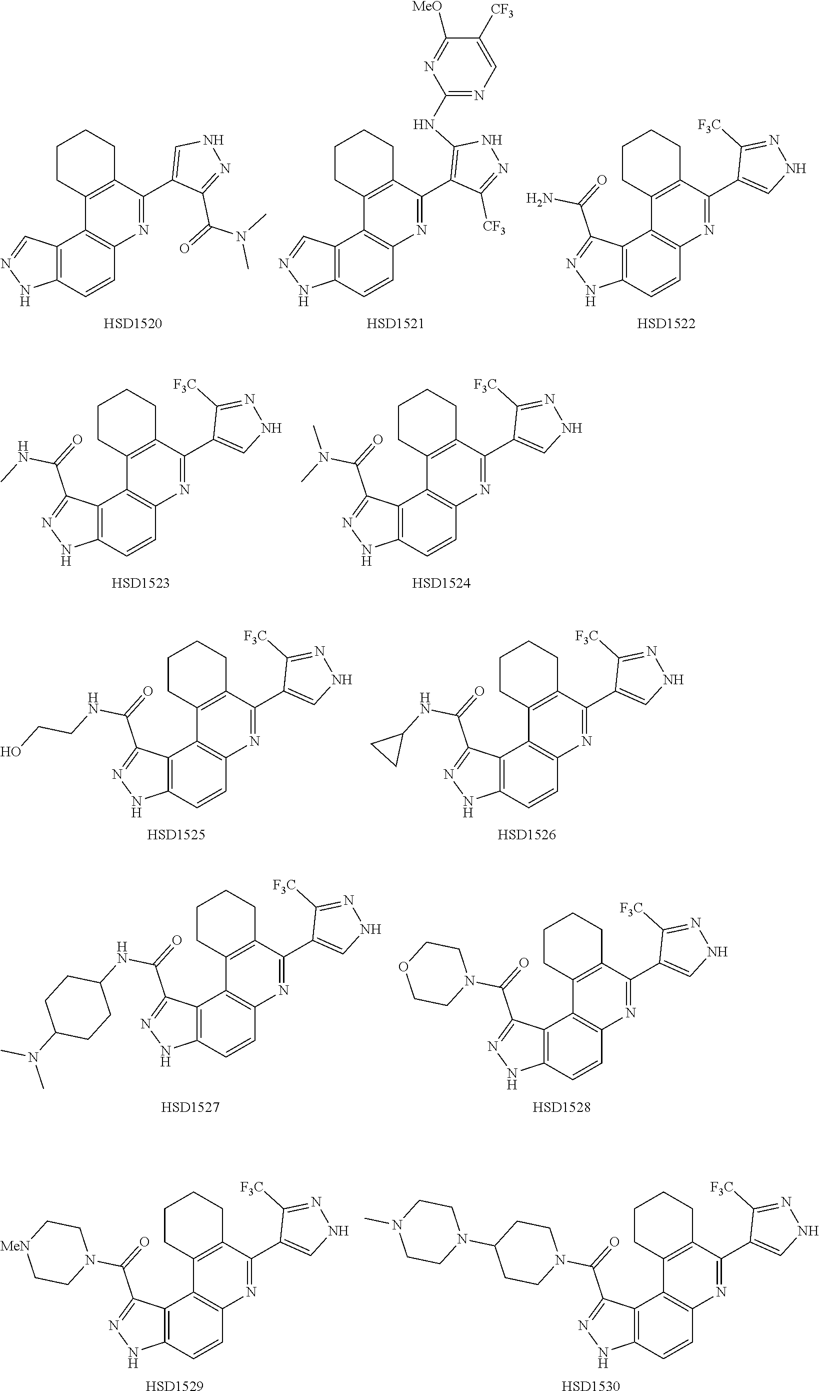

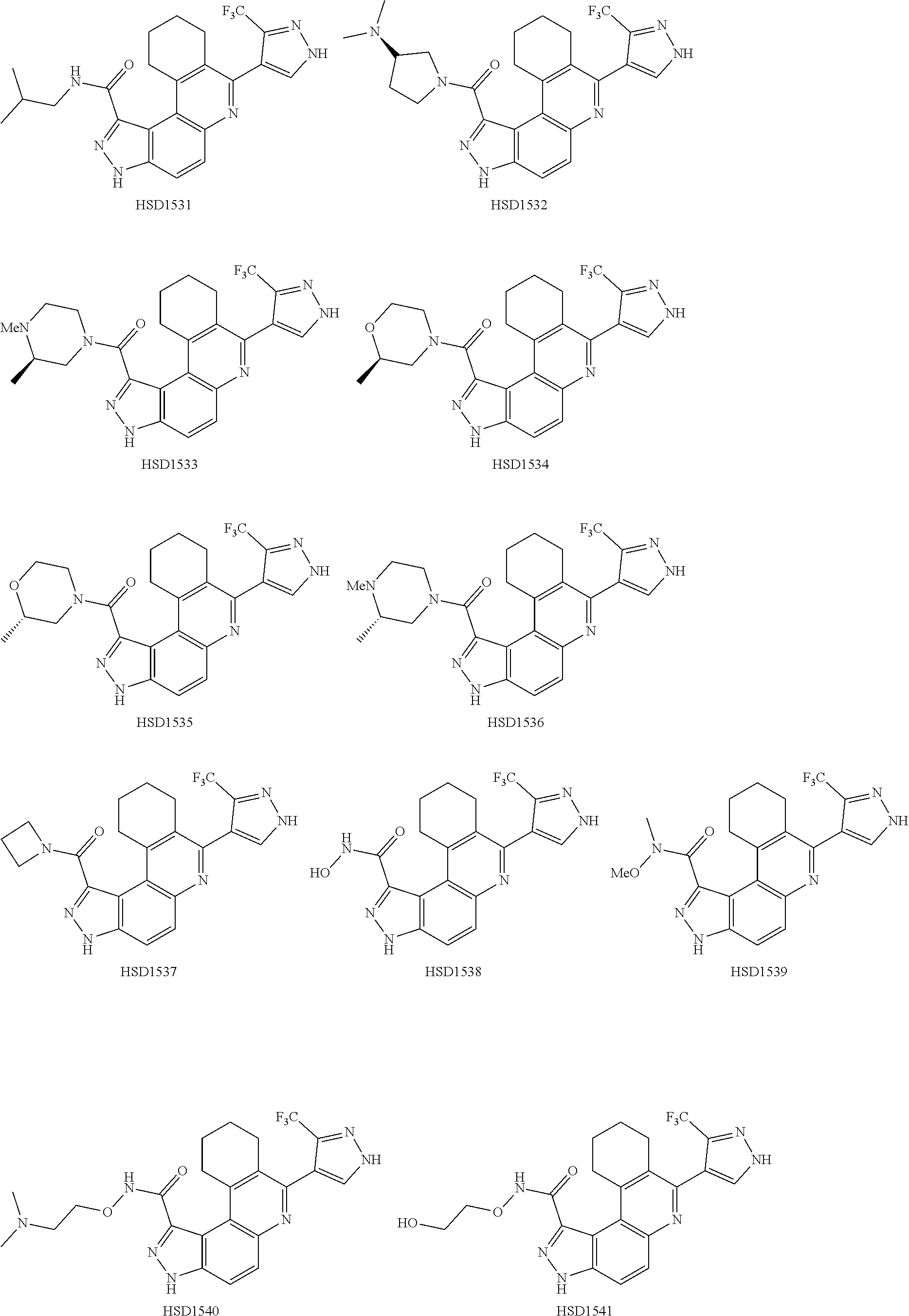

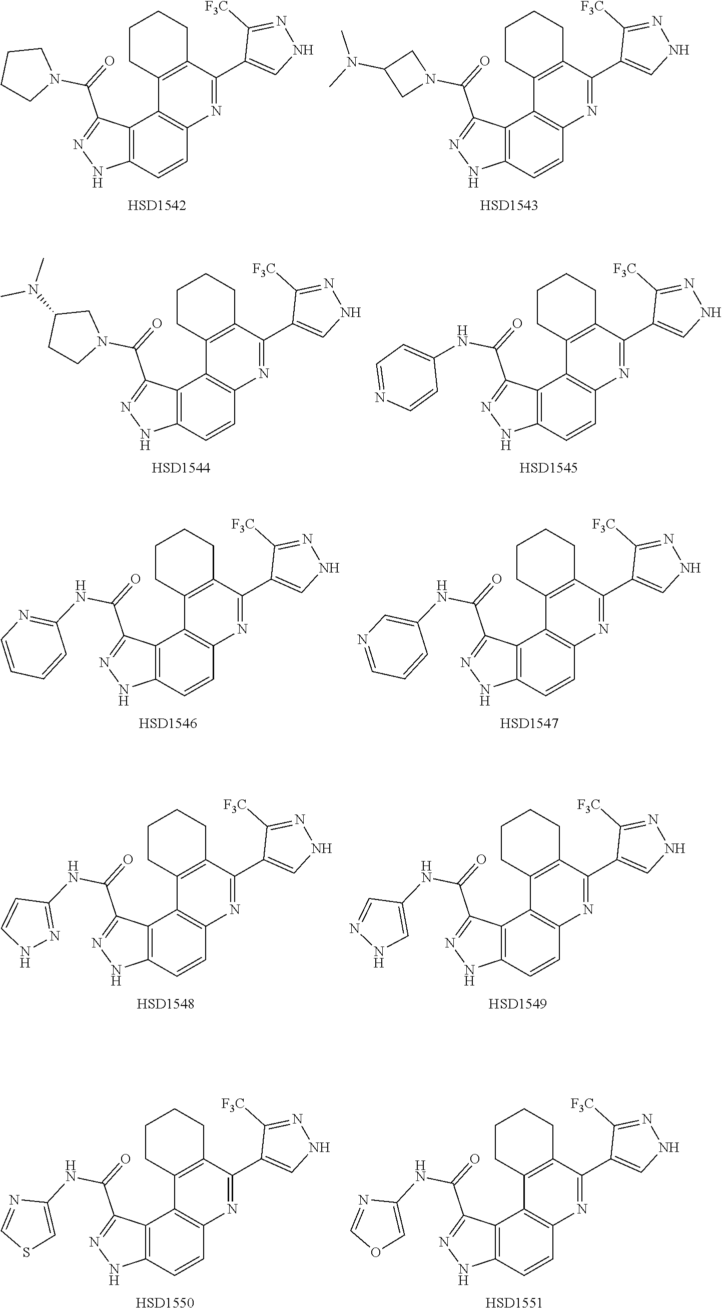

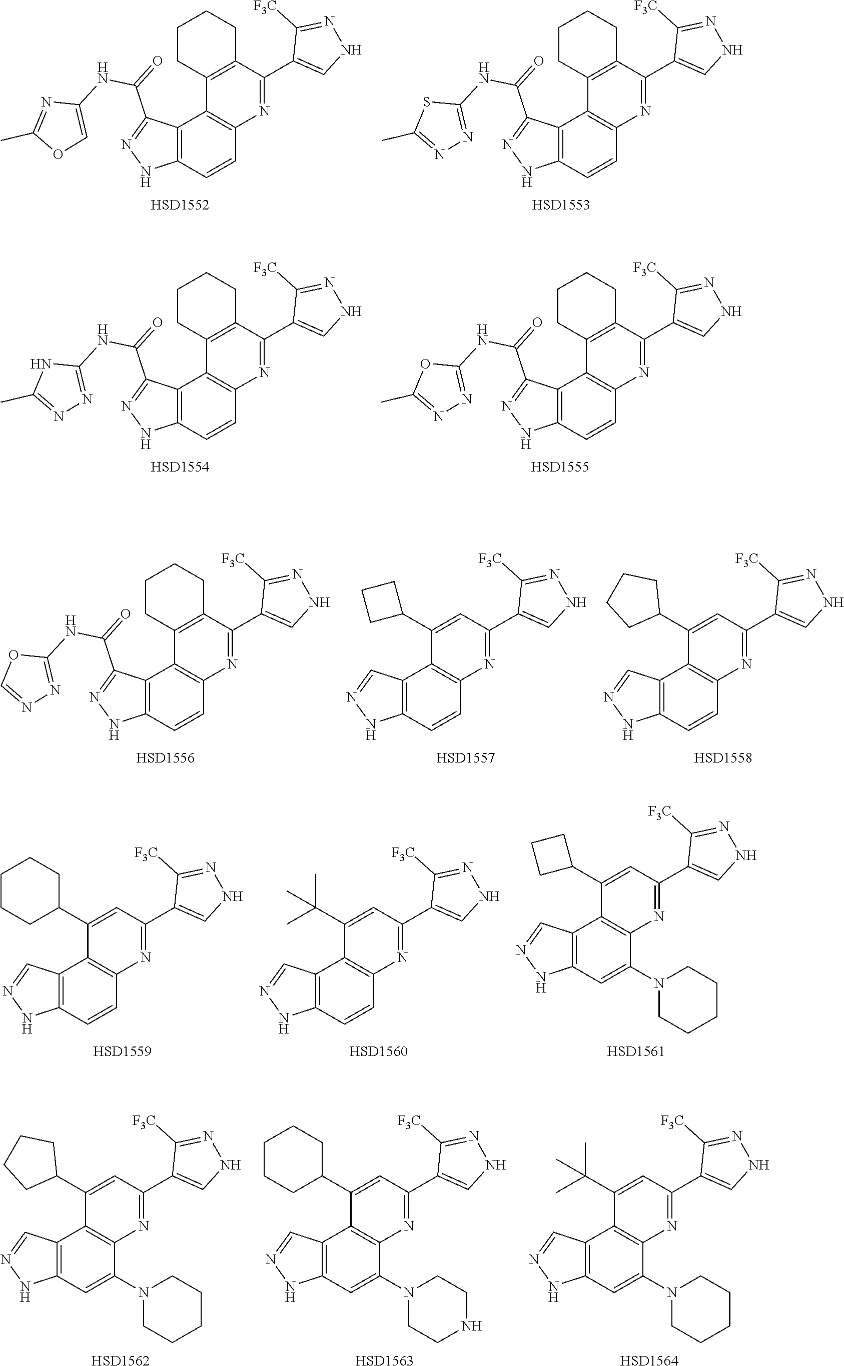

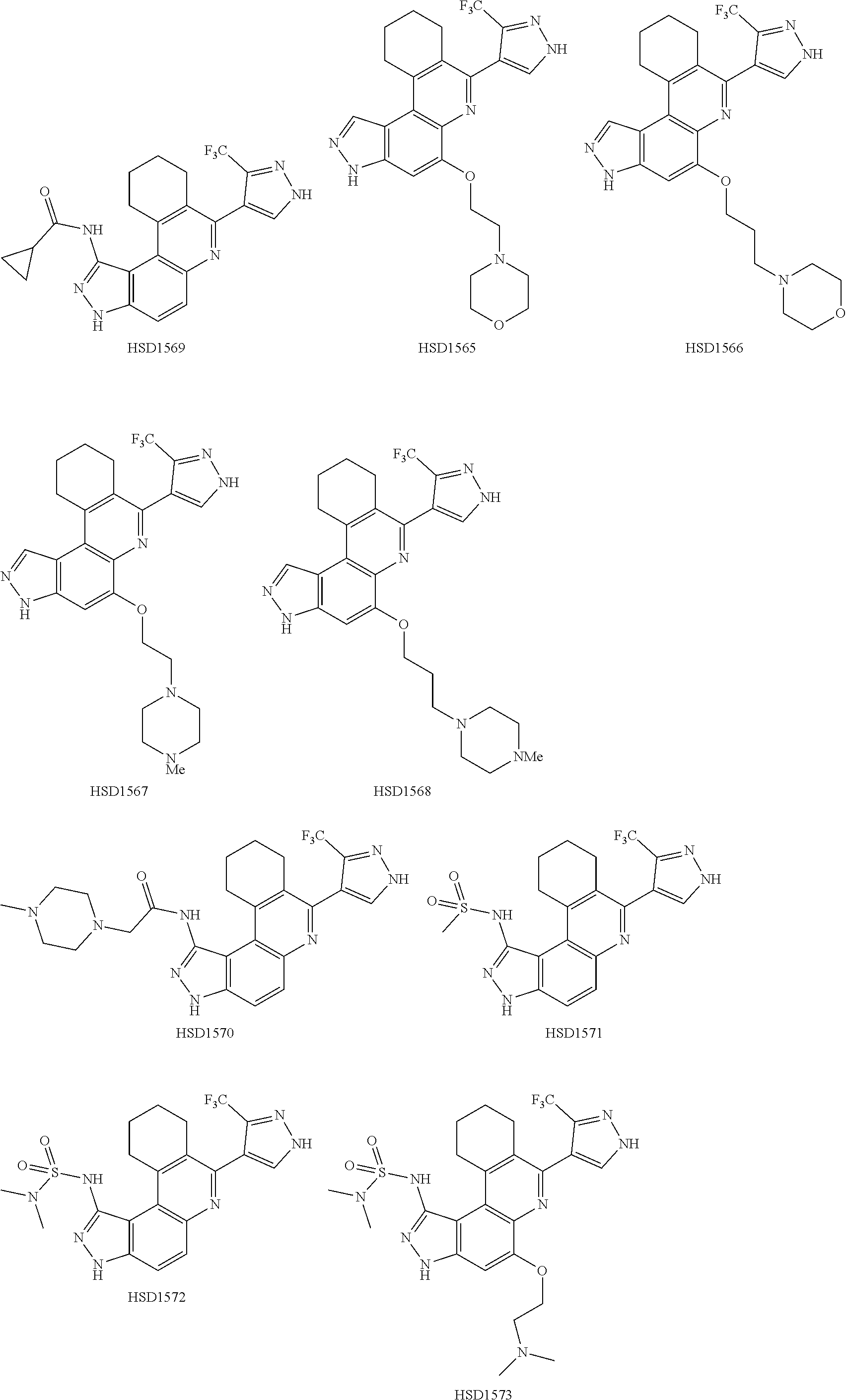

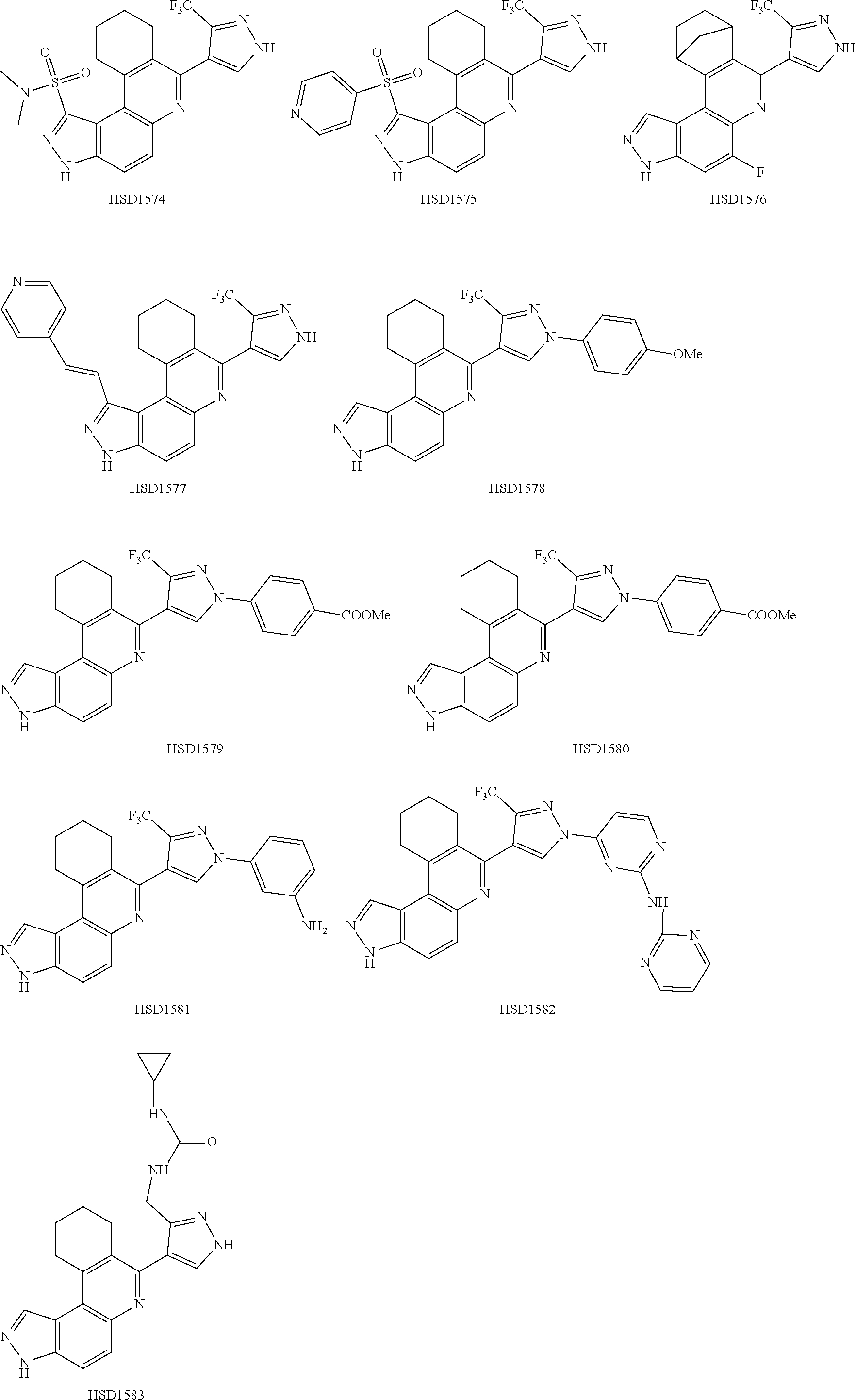

14. A pharmaceutical composition comprising one or more compounds of claims 1˜15, or a, pharmaceutically acceptable salt thereof, together with one or more diluents, excipients or carriers.

15. The compound according to claims 1˜13 is a histone demethylase inhibitor.

16. The compound according to claims 1˜13 is a kinase inhibitor.

17. The kinase inhibitor according to claim 16, wherein the kinase is selected from the group consisting of FLT3, MNK1/2, JAK1/2/3, Limk1/2, various CDKs, Haspin, ROCK1/2, TOPK, LRRK2, GSK3a/3b, RSK1-4, ERK, P70S6K, AKT, PI3K, p38, PKC, PKA, FGFR1-4, VEGFR1-3, ALK, AXL, LIMK1/2, Aurora A/B, ABL1, AKT, CSF1R, CSNK1D, DCAMKL1, CSNK1G2, EPHA2, ERBB2, IKK-alpha, IKK-beta, JNK1/2/3, MARK3, MEK1/2, MET, MLK1, PAK1/2/4, PDGFRa/b, PIM1/2/3, PLK1/2/3/4, PRKCE, PRKX, RET, TAOK2, TRKA/B/C, ULK2, and receptor-interacting protein kinase 4 (RIPK4).

18. A method for treating diseases mediated by a kinase and/or histone demethylases, including inflammation, cancer, viral and bacterial infections, gastrointestinal disorders, eye diseases, neurological, cardiovascular and immunological disorders, comprising the step of administering a therapeutically effective amount of one or more compounds of claims 1˜13, and one or more carriers, diluents, or excipients, to a patient in need of relief from said cancer.

19. A method for treating diseases mediated by a kinase and histone demethylases, including inflammation, cancer, viral and bacterial infections, gastrointestinal disorders, eye diseases, neurological, cardiovascular and immunological disorders, comprising the step of administering a therapeutically effective amount of a compound of claims 1˜13 in combination with one or more other compounds of the same or different mode of action, and one or more carriers, diluents, or excipients, to a patient in need of relief from said cancer.

20. A drug conjugate, either small molecule or biologic conjugate, comprising one or more compounds of claims 1˜13, wherein the conjugate confers cell-type or tissue type targeting or the conjugate targets another pathway that synergizes the action of compounds of claims 1˜13.

21. A drug conjugate, either small molecule or biologic conjugate, comprising one or more compounds of claims 1˜13, wherein the conjugate confers aqueous solubility or low clearance.

22. A drug conjugate containing one or more compounds of claims 1˜13, and a moiety that aids the degradation of target proteins via systems including but not limited to the ubiquitin ligase/proteosome degradation system.

23. A pharmaceutical composition comprising nanoparticles of one or more compounds of claims 1˜13, together with one or more diluents, excipients or carriers.

24. A prodrug comprising one or more compounds of claims 1˜13, wherein the prodrug moiety is removed at specific location, such as gastrointestinal or in blood or in tissues or in cancer specific.

25. Analogs of compounds in claims 1˜13 whereby specific metabolic hot spots are modified with groups such as deuterium or fluorine.

26. Use of a compound or a pharmaceutically acceptable salt thereof according to claims 1˜13 in the manufacture of a medicament for treating cancer in a subject.
